(19) United States
(12) Patent Application Publication
(10) Pub. No.: US 2024/0201205 A1
(43) Pub. Date: Jun. 20, 2024

(54) METHOD FOR DETECTING NEUROLOGICAL DISEASES USING SUPRAMOLECULE POLYMER THERAPEUTICS

(71) Applicant: The Regents of the University of California, Oakland, CA (US)

(72) Inventors: Stanley B. Prusiner, San Francisco, CA (US); Nick A. Paras, San Francisco, CA (US); Jay Conrad, San Francisco, CA (US); Ny Sin, San Francisco, CA (US); Sandeep Raikar, San Francisco, CA (US); Mark Vander Wal, San Francisco, CA (US); Jean-Marc M. Grandjean, San Francisco, CA (US); Shigeo Yamanoi, San Francisco, CA (US); Masahiro Inoue, San Francisco, CA (US); Shimpei Hirano, San Francisco, CA (US); Masatoshi Honzumi, San Francisco, CA (US); Osamu Iwamoto, San Francisco, CA (US); Koji Sasaki, San Francisco, CA (US); Yamato Suzuki, San Francisco, CA (US); Atsushi Tengeiji, San Francisco, CA (US); Daniel R. Southworth, San Francisco, CA (US); Darren Hutt, San Francisco, CA (US); Jacob I. Ayers, San Francisco, CA (US); Steven H. Olson, San Francisco, CA (US); Cuong Quoc Ly, San Francisco, CA (US); John West, San Francisco, CA (US); Gregory E. Merz, San Francisco, CA (US); Helene Viart, San Francisco, CA (US)

(73) Assignee: The Regents of the University of California, Oakland, CA (US)

(21) Appl. No.: 18/468,639

(22) Filed: Sep. 15, 2023

Related U.S. Application Data
(60) Provisional application No. 63/407,106, filed on Sep. 15, 2022.

(51) Int. Cl. G01N 33/68; G01N 33/58

(52) U.S. Cl. CPC G01N 33/6896; G01N 33/58; G01N 2800/2821; G01N 2800/2835

(57) ABSTRACT

A method for detecting a neurological disease the method comprising isolating brain tissue from its natural environment, contacting the brain tissue with a labeled molecule which binds to multiple sites of stacked proteins associated with neurodegenerative disease, determining the binding of the labeled molecule; and thereby determining a neurodegenerative disease associated with the brain tissue.

### CROSS-REFERENCE TO RELATED APPLICATIONS

This Pursuant to 35 U.S.C. § 119 (e), this application claims priority to the filing date of U.S. Provisional Patent Application Ser. No. 63/407,106 filed Sep. 15, 2022, the disclosure of which application is herein incorporated by reference.

### GOVERNMENT SUPPORT

This invention was made with Government support under grant no. 66721 awarded by the Henry M. Jackson Foundation for the Uniformed Services University of the Health Services (USU) and grant no. P01AG002132 awarded by the National Institutes of Health. The Government has certain rights in the invention.

### REFERENCE TO AN ELECTRONIC SEQUENCE LISTING

A sequence listing is provided herewith as a sequence listing xml, “UCSF-691US2_Seq_List.xml” created on Sep. 14, 2023, and having a size of 11,439 Bytes. The contents of the sequence listing xml are incorporated by reference herein in their entirety.

### FIELD OF THE INVENTION

The invention relates generally to the field of neurodegenerative diseases and more particularly to drug/amyloid fibril complexes, and methods of treating and detecting amyloid fibril diseases and associating particular propagating, beta-sheet-rich protein confirmations (prions) with particular types of neurodegenerative diseases.

### BACKGROUND OF THE INVENTION

More than 200 years ago, James Parkinson described the disease that bears his name (2). Little progress was made in deciphering the cause of Parkinson's disease (PD) for nearly a century before Fritz Lewy discovered inclusions in the brain that were named after him (3). In his initial manuscript, he described these inclusions as eosinophilic and insoluble in alcohol, chloroform, and benzene, consistent with the presence of a major protein component. Two years later Konstantin Tretiakoff described the abundance of these inclusions in the substantia nigra in PD and named them Lewy bodies (4).

Drugs generally form discrete molecular associations with a target site that result in a therapeutic interaction. This occurs when drugs bind to one or more biological targets based on complementary shape, character and/or the reactivity of their surfaces. In some cases, the result is a binary drug-target complex which alters the conformation, activity or fate of the target. In still others, multiple (identical or different) small molecules bind co-operatively at multiple discrete positions within a target complex with a net affinity or effect that is greater than the sum of individual binding events (e.g. in GPCRs: Lu, et al. Structural basis for the cooperative allosteric activation of the free fatty acid receptor GPR40 (2017) Nat Struct Mol Biol 24, 570-577; type III kinase inhibitors Martinez, et al. (2020). Avoiding or Co-Opting ATP Inhibition: Overview of Type III, IV, V, and VI Kinase Inhibitors. In: Shapiro, P. (eds) Next Generation Kinase Inhibitors. Springer, Cham.)

In rare cases, two or three molecules of the same ligand have been observed to bind within a singular site in a target complex while engaging in productive interactions with each other (supramolecular dimer of small molecule ligands: Shokat, K. M. A drug-drug interaction crystallizes a new entry point into the UPR. Mol. Cell 38, 161-163 (2010); supramolecular trimer of ligands: Stornaiuolo, M., De Kloe, G., Rucktooa, P. et al. Assembly of a π-π stack of ligands in the binding site of an acetylcholine-binding protein. Nat Commun 4, 1875 (2013). dimers leveraged for drug discovery: Allen, et al. bioRxiv 2022.05.23.493001, https://doi.org/10.1101/2022.05.23.493001.) The reversible intermolecular interactions between monomers in these supramolecular dimeric or trimeric complexes counter the entropic penalty for binding two distinct molecules in the same site at the same time.

Supramolecular polymers assemble from monomers spontaneously from appropriately disposed monomers and maintain their polymeric properties in solution (de Greef, T., Meijer, E. Supramolecular polymers. Nature 453, 171-173 (2008). They are distinguished from supramolecular dimers and trimers by an increased number of monomer subunits which self-associate.

We unexpectedly observed the supramolecular polymer assembly of an α-synuclein prion inhibitor bound to amyloid fibrils of α-synuclein (FIG. 1). Based on that observation we propose that supramolecular polymers of small molecule monomers are uniquely suited to interact with the supramolecular assemblies of proteins which feature in degenerative diseases of the central nervous system (CNS). Owing to their helical symmetry, fibrillar oligomers and polymers of proteins that feature in neurodegenerative disease (e.g. α-synuclein, tau, amyloid beta, etc.) typically lack discrete binding pockets which are sufficiently concave that they might engage in potent binding interactions with small molecule ligands. In lieu of such druggable binding pockets, these fibrillar protein assemblies present channels created by the repeated display of surfaces and voids along the long axis of the fibril. Drugs and diagnostic ligands which are large enough to span relatively long distances along binding channels would be able to produce sufficient affinity to effect a pharmacologically relevant change in the activity of the fibrillized protein, but such large molecules are not typically permeable enough to traverse the blood-brain barrier. We have identified supramolecular polymers composed of low molecular weight monomers which can pass through the blood-brain barrier and assemble in the presence of the ordered α-synuclein aggregates that feature in multiple system atrophy (MSA). We have demonstrated the generality of our approach to other proteins that feature in neurodegenerative disease by establishing the binding of another supramolecular polymer: “poly GTP-1” to paired helical filaments of tau from Alzheimer's disease (FIG. 2). As such, we have identified a general class of supramolecular polymers as modifiers and diagnostic agents for the treatment of diseases such as Alzheimer's disease, Parkinson's disease and MSA which feature the ordered accumulation of misfolded proteins.

### GENERAL INFORMATION

Before the present methods and uses are described, it is to be understood that this invention is not limited to particular steps, devices and compounds described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supersedes any disclosure of an incorporated publication to the extent there is a contradiction.

It must be noted that as used herein and in the appended claims, the singular forms “a”, “an”, and “the” include plural referents unless the context clearly dictates otherwise. Thus, for example, reference to “a scan” includes a plurality of such scans and reference to “the Biomarker of Response” includes reference to one or more such Biomarkers of Response and equivalents thereof known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed.

All patents and publications, including all sequences disclosed within such patents and publications, referred to herein are expressly incorporated by reference.

### BRIEF SUMMARY OF THE INVENTION

A method of inhibiting propagation of protein misfolding associated with a neurological disease, is carried out by contacting an environment populated with a propagating amyloid conformation of a protein (prion) associated with a neurological disease with molecules which bind multiple adjacent sites of the protein assemblies and allowing the molecules to bind multiple cites of the protein assemblies; and thereby impeding propagation of the disease-associated conformation of the protein in the environment. Drug/prion complexes are formed and uses of the drugs in detection and treatment of neurodegenerative diseases are disclosed.

A method of interrupting propagation of stacked proteins associated with a neurological disease, is carried out by contacting an environment populated with stacked proteins associated with a neurological disease with molecules which binds multiple sites of the stacked proteins and allowing the molecules to bind multiple cites of the stacked proteins; and thereby impeding propagation of the stacked proteins in the environment. Drug/prion complexes are formed and uses of the drugs in detection and treatment of neurodegenerative diseases are disclosed

A method of impeding progressive, templated misfolding of proteins associated with neurodegenerative disease, comprising administering molecules into a biological milieu containing both a propagating amyloid conformation of a protein and a native cellular form of the same protein; allowing for formation of a complex between a supramolecular polymer assembly of the molecules and a supramolecular assembly of the protein; and thereby impeding further sequestration of the native cellular protein and its conversion to the propagating amyloid form. The biological milieu may be selected from the group consisting of a cell lysate, an active cell culture, and a mammalian brain, and may be an animal model brain or a human brain.

Drug/prion complexes are formed and uses of the drugs in detection and treatment of neurodegenerative diseases are disclosed. The drug may be a supra-molecular polymer which simultaneously binds to multiple stacked prions sites, or a plurality of small molecules provided such that each small molecule binds at different sites in a stacked prion complex thereby forming Drug/prion complexes. By tagging the supra-molecular polymer or small molecule which bind the stacked prion complex it is possible to provide for detection and by administering therapeutically effective amounts it is possible to provide a therapy in that drug-target complex alters the conformation, activity or fate of the target hindering replication.

Compounds are also provided for use in the methods disclosed herein.

### BRIEF DESCRIPTION OF THE DRAWINGS

The invention is best understood from the following detailed description when read in conjunction with the accompanying drawings. It is emphasized that, according to common practice, the various features of the drawings are not to scale. On the contrary, the dimensions of the various features are arbitrarily expanded or reduced for clarity. Included in the drawings are the following figures:

FIG. 1 consists of FIGS. 1A, 1B and 1C. FIG. 1A shows an observed electron density in a co-structure of supramolecular polymeric inhibitor (monomer=compound 79) of α-synuclein aggregate propagation bound to fibrils isolated from an MSA patient sample, solved by cryoelectronic microscopy. FIG. 1B shows an observed electron density in a co-structure of supramolecular polymeric inhibitor (monomer=compound 83) of α-synuclein aggregate propagation bound to fibrils isolated from an MSA patient sample, solved by cryoelectronic microscopy. FIG. 1C shows an observed electron density in a co-structure of supramolecular polymeric inhibitor (monomer=compound 125) of α-synuclein aggregate propagation bound to fibrils isolated from an MSA patient sample, solved by cryoelectronic microscopy.

FIG. 2A-2B. FIG. 2A consists of two images showing the observed electron density in a co-structure of a supramolecular polymeric ligand of tau (GTP-1) with a paired helical filament (tau) from an AD patient sample. FIG. 2B consists of two images showing the observed electron density in a co-structure of a supramolecular polymeric ligand of tau (GTP-1 polymer) with a paired helical filament from an AD patient sample.

FIG. 3 is a conceptual view of planar cores of a binding portion of a molecule which binds to a target site on the stacked proteins of FIG. 3 showing the interplanar distance between cores at 3.3 to 3.5 angstroms.

FIG. 4 shows the planar core of two molecules as shown in FIG. 4 also showing the distance between equivalent atoms in adjacent molecules at 4.8 angstroms and the angle between the line defined by two equivalent atoms on adjacent molecules and a line perpendicular to the planar cores at 44°.

FIG. 5 shows the planar cores as shown in FIGS. 4 and 5 showing the interplanar distance between cores at 3.3-3.5 angstroms, the distance between equivalent atoms in adjacent molecules at 4.8 angstroms, the angle between the line defined by two equivalent atoms on adjacent molecules and line perpendicular to the planar cores at 44° and in-plane displacement of equivalent atoms.

FIG. 6 which consists of 821 structures in a list of chemical structures numbered 1-821 along with the IUPAC name for each of the structures shown. These compounds are molecules which bind to multiple sites of stacked prion proteins.

FIG. 7 which consists of compounds with EC50 in a cellular assay for the propagation of synuclein fibrils from MSA.

FIG. 8 which consists of compounds that extend the symptom free survival in a mouse model of MSA.

FIG. 9 consists of two images showing the observed electron density of an α-synuclein fibril from an MSA patient and the fibril bound to an inhibitor compound (compound 22; circle bottom image bound to residues 86-99 of α-synuclein SEQ ID NO: 9).

FIG. 10A-10F shows an observed electron density in a co-structure of supramolecular polymeric inhibitor (10A monomer=compound 22; 10B monomer=compound 83; 10C monomer=compound 149; 10D monomer=compound 293; 10E monomer=compound 294; 10F monomer=compound 427) of α-synuclein aggregate propagation bound to fibrils isolated from an MSA patient sample, solved by cryoelectronic microscopy. FIG. 10A discloses a co-structure of supramolecular polymeric inhibitor (monomer=compound 22; arrow) of α-synuclein aggregate propagation bound to fibrils isolated from an MSA patient sample, solved by cryoelectronic microscopy. FIG. 10B discloses a co-structure of supramolecular polymeric inhibitor (monomer=compound 83; arrow) of α-synuclein aggregate propagation bound to fibrils isolated from an MSA patient sample, solved by cryoelectronic microscopy. FIG. 10C discloses a co-structure of supramolecular polymeric inhibitor (monomer=compound 149; arrow) of α-synuclein aggregate propagation bound to fibrils isolated from an MSA patient sample, solved by cryoelectronic microscopy. FIG. 10D discloses a co-structure of supramolecular polymeric inhibitor (monomer=compound 293; arrow) of α-synuclein aggregate propagation bound to fibrils isolated from an MSA patient sample, solved by cryoelectronic microscopy. FIG. 10E discloses a co-structure of supramolecular polymeric inhibitor (monomer=compound 294; arrow) of α-synuclein aggregate propagation bound to fibrils isolated from an MSA patient sample, solved by cryoelectronic microscopy. FIG. 10F discloses a co-structure of supramolecular polymeric inhibitor (monomer=compound 427; arrow) of α-synuclein aggregate propagation bound to fibrils isolated from an MSA patient sample, solved by cryoelectronic microscopy.

FIG. 11 discloses autoradiography of compound 294 in mouse brains inoculated with MSA prions.

### DETAILED DESCRIPTION OF THE INVENTION

### Definitions

As used herein, the term “alkyl” by itself or as part of another substituent refers to a saturated branched or straight-chain monovalent hydrocarbon radical derived by the removal of one hydrogen atom from a single carbon atom of a parent alkane. Typical alkyl groups include, but are not limited to, methyl; ethyl, propyls such as propan-1-yl or propan-2-yl; and butyls such as butan-1-yl, butan-2-yl, 2-methyl-propan-1-yl or 2-methyl-propan-2-yl. In some embodiments, an alkyl group comprises from 1 to 20 carbon atoms. In other embodiments, an alkyl group comprises from 1 to 10 carbon atoms. In still other embodiments, an alkyl group comprises from 1 to 6 carbon atoms, such as from 1 to 4 carbon atoms.

“Alkanyl” by itself or as part of another substituent refers to a saturated branched, straight-chain or cyclic alkyl radical derived by the removal of one hydrogen atom from a single carbon atom of an alkane. Typical alkanyl groups include, but are not limited to, methanyl; ethanyl; propanyls such as propan-1-yl, propan-2-yl (isopropyl), cyclopropan-1-yl, etc.; butanyls such as butan-1-yl, butan-2-yl (sec-butyl), 2-methyl-propan-1-yl (isobutyl), 2-methyl-propan-2-yl (t-butyl), cyclobutan-1-yl, etc.; and the like.

“Alkylene” refers to a branched or unbranched saturated hydrocarbon chain, usually having from 1 to 40 carbon atoms, more usually 1 to 10 carbon atoms and even more usually 1 to 6 carbon atoms. This term is exemplified by groups such as methylene (—CH₂—), ethylene (—CH₂CH₂—), the propylene isomers (e.g., —CH₂CH₂CH₂— and —CH(CH₃)CH₂—) and the like.

“Alkenyl” by itself or as part of another substituent refers to an unsaturated branched, straight-chain or cyclic alkyl radical having at least one carbon-carbon double bond derived by the removal of one hydrogen atom from a single carbon atom of an alkene. The group may be in either the cis or trans conformation about the double bond(s). Typical alkenyl groups include, but are not limited to, ethenyl; propenyls such as prop-1-en-1-yl, prop-1-en-2-yl, prop-2-en-1-yl (allyl), prop-2-en-2-yl, cycloprop-1-en-1-yl; cycloprop-2-en-1-yl; butenyls such as but-1-en-1-yl, but-1-en-2-yl, 2-methyl-prop-1-en-1-yl, but-2-en-1-yl, but-2-en-1-yl, but-2-en-2-yl, buta-1,3-dien-1-yl, buta-1,3-dien-2-yl, cyclobut-1-en-1-yl, cyclobut-1-en-3-yl, cyclobuta-1,3-dien-1-yl, etc.; and the like.

“Alkynyl” by itself or as part of another substituent refers to an unsaturated branched, straight-chain or cyclic alkyl radical having at least one carbon-carbon triple bond derived by the removal of one hydrogen atom from a single carbon atom of an alkyne. Typical alkynyl groups include, but are not limited to, ethynyl; propynyls such as prop-1-yn-1-yl, prop-2-yn-1-yl, etc.; butynyls such as but-1-yn-1-yl, but-1-yn-3-yl, but-3-yn-1-yl, etc.; and the like.

“Acyl” by itself or as part of another substituent refers to a radical —C(O)R³⁰, where R³⁰ is hydrogen, alkyl, cycloalkyl, cycloheteroalkyl, aryl, arylalkyl, heteroalkyl, heteroaryl, heteroarylalkyl as defined herein and substituted versions thereof. Representative examples include, but are not limited to formyl, acetyl, cyclohexylcarbonyl, cyclohexylmethylcarbonyl, benzoyl, benzylcarbonyl, piperonyl, propionyl, succinyl, and malonyl, and the like.

The term “aminoacyl” refers to the group —C(O)NR²¹R²², wherein R²¹ and R²² independently are selected from the group consisting of hydrogen, alkyl, substituted alkyl, alkenyl, substituted alkenyl, alkynyl, substituted alkynyl, aryl, substituted aryl, cycloalkyl, substituted cycloalkyl, cycloalkenyl, substituted cycloalkenyl, heteroaryl, substituted heteroaryl, heterocyclic, and substituted heterocyclic and where R²¹ and R²² are optionally joined together with the nitrogen bound thereto to form a heterocyclic or substituted heterocyclic group, and wherein alkyl, substituted alkyl, alkenyl, substituted alkenyl, alkynyl, substituted alkynyl, cycloalkyl, substituted cycloalkyl, cycloalkenyl, substituted cycloalkenyl, aryl, substituted aryl, heteroaryl, substituted heteroaryl, heterocyclic, and substituted heterocyclic are as defined herein.

“Alkoxy” by itself or as part of another substituent refers to a radical —OR³¹ where R³¹ represents an alkyl or cycloalkyl group as defined herein. Representative examples include, but are not limited to, methoxy, ethoxy, propoxy, butoxy, cyclohexyloxy and the like.

“Alkoxycarbonyl” by itself or as part of another substituent refers to a radical —C(O)OR³¹ where R³¹ represents an alkyl or cycloalkyl group as defined herein. Representative examples include, but are not limited to, methoxycarbonyl, ethoxycarbonyl, propoxycarbonyl, butoxycarbonyl, cyclohexyloxycarbonyl and the like.

“Aryl” by itself or as part of another substituent refers to a monovalent aromatic hydrocarbon radical derived by the removal of one hydrogen atom from a single carbon atom of an aromatic ring system. Typical aryl groups include, but are not limited to, groups derived from aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like. In certain embodiments, an aryl group comprises from 6 to 20 carbon atoms. In certain embodiments, an aryl group comprises from 6 to 12 carbon atoms. Examples of an aryl group are phenyl and naphthyl.

“Arylalkyl” by itself or as part of another substituent refers to an acyclic alkyl radical in which one of the hydrogen atoms bonded to a carbon atom, typically a terminal or sp a carbon atom, is replaced with an aryl group. Typical arylalkyl groups include, but are not limited to, benzyl, 2-phenylethan-1-yl, 2-phenylethen-1-yl, naphthylmethyl, 2-naphthylethan-1-yl, 2-naphthylethen-1-yl, naphthobenzyl, 2-naphthophenylethan-1-yl and the like. Where specific alkyl moieties are intended, the nomenclature arylalkanyl, arylalkenyl and/or arylalkynyl is used. In certain embodiments, an arylalkyl group is (C₇-C₃₀) arylalkyl, e.g., the alkanyl, alkenyl or alkynyl moiety of the arylalkyl group is (C₁-C₁₀) and the aryl moiety is (C₆-C₂₀). In certain embodiments, an arylalkyl group is (C₇-C₂₀) arylalkyl, e.g., the alkanyl, alkenyl or alkynyl moiety of the arylalkyl group is (C₁-C₈) and the aryl moiety is (C₆-C₁₂).

“Arylaryl” by itself or as part of another substituent, refers to a monovalent hydrocarbon group derived by the removal of one hydrogen atom from a single carbon atom of a ring system in which two or more identical or non-identical aromatic ring systems are joined directly together by a single bond, where the number of such direct ring junctions is one less than the number of aromatic ring systems involved. Typical arylaryl groups include, but are not limited to, biphenyl, triphenyl, phenyl-napthyl, binaphthyl, biphenyl-napthyl, and the like. When the number of carbon atoms in an arylaryl group are specified, the numbers refer to the carbon atoms comprising each aromatic ring. For example, (C₅-C₁₄) arylaryl is an arylaryl group in which each aromatic ring comprises from 5 to 14 carbons, e.g., biphenyl, triphenyl, binaphthyl, phenylnapthyl, etc. In certain embodiments, each aromatic ring system of an arylaryl group is independently a (C₅-C₁₄) aromatic. In certain embodiments, each aromatic ring system of an arylaryl group is independently a (C₅-C₁₀) aromatic. In certain embodiments, each aromatic ring system is identical, e.g., biphenyl, triphenyl, binaphthyl, trinaphthyl, etc.

“Cycloalkyl” by itself or as part of another substituent refers to a saturated or unsaturated cyclic alkyl radical. Where a specific level of saturation is intended, the nomenclature “cycloalkanyl” or “cycloalkenyl” is used. Typical cycloalkyl groups include, but are not limited to, groups derived from cyclopropane, cyclobutane, cyclopentane, cyclohexane and the like. In certain embodiments, the cycloalkyl group is (C₃-C₁₀) cycloalkyl. In certain embodiments, the cycloalkyl group is (C₃-C₇) cycloalkyl.

“Cycloheteroalkyl” or “heterocyclyl” by itself or as part of another substituent, refers to a saturated or unsaturated cyclic alkyl radical in which one or more carbon atoms (and any associated hydrogen atoms) are independently replaced with the same or different heteroatom. Typical heteroatoms to replace the carbon atom(s) include, but are not limited to, N, P, O, S, Si, etc. Where a specific level of saturation is intended, the nomenclature “cycloheteroalkanyl” or “cycloheteroalkenyl” is used. Typical cycloheteroalkyl groups include, but are not limited to, groups derived from epoxides, azirines, thiiranes, imidazolidine, morpholine, piperazine, piperidine, pyrazolidine, pyrrolidine, quinuclidine and the like.

“Heteroalkyl, Heteroalkanyl, Heteroalkenyl and Heteroalkynyl” by themselves or as part of another substituent refer to alkyl, alkanyl, alkenyl and alkynyl groups, respectively, in which one or more of the carbon atoms (and any associated hydrogen atoms) are independently replaced with the same or different heteroatomic groups. Typical heteroatomic groups which can be included in these groups include, but are not limited to, —O—, —S—, —S—S—, —O—S—, —NR³⁷R³⁸—, ·═N—N═, —N═N—, —N═N—NR³⁹R⁴⁰, —PR⁴¹—, —P(O)₂—, —POR⁴²—, —O—P(P)₂—, —S—O—, —S—(O)—, —SO₂—, —SnR⁴³R⁴⁴— and the like, where R³⁷, R³⁸, R³⁹, R⁴⁰, R⁴¹, R⁴², R⁴³ and R⁴⁴ are independently hydrogen, alkyl, substituted alkyl, aryl, substituted aryl, arylalkyl, substituted arylalkyl, cycloalkyl, substituted cycloalkyl, cycloheteroalkyl, substituted cycloheteroalkyl, heteroalkyl, substituted heteroalkyl, heteroaryl, substituted heteroaryl, heteroarylalkyl or substituted heteroarylalkyl.

“Heteroaryl” by itself or as part of another substituent, refers to a monovalent heteroaromatic radical derived by the removal of one hydrogen atom from a single atom of a heteroaromatic ring system. Typical heteroaryl groups include, but are not limited to, groups derived from acridine, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene, benzodioxole and the like. In certain embodiments, the heteroaryl group is from 5-20 membered heteroaryl. In certain embodiments, the heteroaryl group is from 5-10 membered heteroaryl. In certain embodiments, heteroaryl groups are those derived from thiophene, pyrrole, benzothiophene, benzofuran, indole, pyridine, quinoline, imidazole, oxazole and pyrazine.

“Heteroarylalkyl” by itself or as part of another substituent, refers to an acyclic alkyl radical in which one of the hydrogen atoms bonded to a carbon atom, typically a terminal or sp³ carbon atom, is replaced with a heteroaryl group. Where specific alkyl moieties are intended, the nomenclature heteroarylalkanyl, heteroarylalkenyl and/or heterorylalkynyl is used. In certain embodiments, the heteroarylalkyl group is a 6-30 membered heteroarylalkyl, e.g., the alkanyl, alkenyl or alkynyl moiety of the heteroarylalkyl is 1-10 membered and the heteroaryl moiety is a 5-20-membered heteroaryl. In certain embodiments, the heteroarylalkyl group is 6-20 membered heteroarylalkyl, e.g., the alkanyl, alkenyl or alkynyl moiety of the heteroarylalkyl is 1-8 membered and the heteroaryl moiety is a 5-12-membered heteroaryl.

“Aromatic Ring System” by itself or as part of another substituent, refers to an unsaturated cyclic or polycyclic ring system having a conjugated π electron system. Specifically included within the definition of “aromatic ring system” are fused ring systems in which one or more of the rings are aromatic and one or more of the rings are saturated or unsaturated, such as, for example, fluorene, indane, indene, phenalene, etc. Typical aromatic ring systems include, but are not limited to, aceanthrylene, acenaphthylene, acephenanthrylene, anthracene, azulene, benzene, chrysene, coronene, fluoranthene, fluorene, hexacene, hexaphene, hexalene, as-indacene, s-indacene, indane, indene, naphthalene, octacene, octaphene, octalene, ovalene, penta-2,4-diene, pentacene, pentalene, pentaphene, perylene, phenalene, phenanthrene, picene, pleiadene, pyrene, pyranthrene, rubicene, triphenylene, trinaphthalene and the like.

“Heteroaromatic Ring System” by itself or as part of another substituent, refers to an aromatic ring system in which one or more carbon atoms (and any associated hydrogen atoms) are independently replaced with the same or different heteroatom. Typical heteroatoms to replace the carbon atoms include, but are not limited to, N, P, O, S, Si, etc. Specifically included within the definition of “heteroaromatic ring systems” are fused ring systems in which one or more of the rings are aromatic and one or more of the rings are saturated or unsaturated, such as, for example, arsindole, benzodioxan, benzofuran, chromane, chromene, indole, indoline, xanthene, etc. Typical heteroaromatic ring systems include, but are not limited to, arsindole, carbazole, β-carboline, chromane, chromene, cinnoline, furan, imidazole, indazole, indole, indoline, indolizine, isobenzofuran, isochromene, isoindole, isoindoline, isoquinoline, isothiazole, isoxazole, naphthyridine, oxadiazole, oxazole, perimidine, phenanthridine, phenanthroline, phenazine, phthalazine, pteridine, purine, pyran, pyrazine, pyrazole, pyridazine, pyridine, pyrimidine, pyrrole, pyrrolizine, quinazoline, quinoline, quinolizine, quinoxaline, tetrazole, thiadiazole, thiazole, thiophene, triazole, xanthene and the like.

“Substituted” refers to a group in which one or more hydrogen atoms are independently replaced with the same or different substituent(s). Typical substituents include, but are not limited to, alkylenedioxy (such as methylenedioxy), -M, —R⁶⁰, —O—, ═O, —OR⁶⁰, —SR⁶⁰, —S—, ═S, —NR⁶⁰R⁶¹, ═NR⁶⁰, —CF₃, —CN, —OCN, —SCN, —NO, —NO₂,
═N₂, —N₃, —S(O)₂O⁻, —S(O)₂OH, —S(O)₂R⁶⁰, —OS(O)₂O⁻, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O—, —C(S)OR⁶⁰, —NR⁶²C (O)NR⁶⁰R⁶¹, —NR⁶²C(S)NR⁶⁰R⁶¹, —NR⁶²C(NR⁶³)NR⁶⁰R⁶¹ and —C(NR⁶²)NR⁶⁰R⁶¹ where M is halogen; R⁶⁰, R⁶¹, R⁶² and R⁶³ are independently hydrogen, alkyl, substituted alkyl, alkoxy, substituted alkoxy, cycloalkyl, substituted cycloalkyl, cycloheteroalkyl, substituted cycloheteroalkyl, aryl, substituted aryl, heteroaryl or substituted heteroaryl, or optionally R⁶⁰ and R⁶¹ together with the nitrogen atom to which they are bonded form a cycloheteroalkyl or substituted cycloheteroalkyl ring; and R⁶ and R⁶⁵ are independently hydrogen, alkyl, substituted alkyl, aryl, cycloalkyl, substituted cycloalkyl, cycloheteroalkyl, substituted cycloheteroalkyl, aryl, substituted aryl, heteroaryl or substituted heteroaryl, or optionally R⁶ and R⁶⁵ together with the nitrogen atom to which they are bonded form a cycloheteroalkyl or substituted cycloheteroalkyl ring. In certain embodiments, substituents include -M, —R⁶⁰, ═O, —OR⁶⁰, —SR⁶⁰, —S—, ═S, —NR⁶⁰R⁶¹, ═R⁶⁰, —CF₃, —CN, —OCN, —SCN, —NO, —NO₂,
═N₂, —N₃, —S(O)₂R⁶⁰, —OS(O)₂O—, —OS(O)₂R⁶⁰, —P(O)(O⁻)₂, —P(O)(OR⁶⁰)(O⁻), —OP(O)(OR⁶⁰)(OR⁶¹), —C(O)R⁶⁰, —C(S)R⁶⁰, —C(O)OR⁶⁰, —C(O)NR⁶⁰R⁶¹, —C(O)O—, —NR⁶²C(O)NR⁶⁰R⁶¹. In certain embodiments, substituents include -M, —R⁶⁰,
═O, —OR⁶⁰, —SR⁶⁰, —NR⁶⁰R⁶¹, —CF₃, —CN, —NO₂, —S(O)₂R⁶⁰, —P(O)(OR⁶⁰)(O⁻), —OP(O)(OR⁶⁰)(OR⁶¹), —C(O)R⁶⁰, —C(O)OR⁶⁰, —C(O)NR⁶⁰R⁶¹, —C(O)O⁻. In certain embodiments, substituents include -M, —R⁶⁰,
═O, —OR⁶⁰, —SR⁶⁰, —NR⁶⁰R⁶¹, —CF₃, —CN, —NO₂, —S(O)₂R⁶⁰, —OP(O)(OR⁶⁰)(OR⁶¹), —C(O)R⁶⁰, —C(O)O R⁶⁰, —C(O)O⁻, where R⁶⁰, R⁶¹ and R⁶² are as defined above. For example, a substituted group may bear a methylenedioxy substituent or one, two, or three substituents selected from a halogen atom, a (1-4C)alkyl group and a (1-4C)alkoxy group.

“Amino” refers to the group —NR^{X}R^{Y} wherein R^{X} and R^{Y} are each independently H or a non-hydrogen substituent. Exemplary non-hydrogen substituents include alkyl groups (e.g. methyl, ethyl, and isopropyl).

“Ether” refers to a diradical group of formula —O—. For instance, if the ether group is connected to an alkyl group, then the overall group is an alkoxy group (e.g. —OCH₃ or methoxy). If the ether is connected to a carbonyl group, then the overall group is an ester group of formula —OC(O)—.

“Halo” and “halogen” refer to the chloro, bromo, fluoro, and iodo groups.

“Nitro” refers to the group of formula —NO₂.

Unless otherwise specified, reference to an atom is meant to include all isotopes of that atom. For example, reference to H includes ¹H, ²H (i.e. D or deuterium) and ³H (i.e. tritium), and reference to C includes both ¹²C and all other isotopes of carbon (e.g. ¹³C). Unless specified otherwise, groups include all possible stereoisomers.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

In certain embodiments, a substituent may contribute to optical isomerism and/or stereo isomerism of a compound. Salts, solvates, hydrates, and prodrug forms of a compound are also of interest. All such forms are embraced by the present disclosure. Thus, the compounds described herein include salts, solvates, hydrates, prodrug and isomer forms thereof, including the pharmaceutically acceptable salts, solvates, hydrates, prodrugs and isomers thereof. In certain embodiments, a compound may be metabolized into a pharmaceutically active derivative.

### Neurodegenerative Diseases

The present disclosure provides methods, drugs, molecules, labeled molecules compounds, and α-synuclein prion inhibitors for detecting and treating neurodegenerative diseases. The terms “neurodegenerative disease” and “neurological disease” may be used interchangeably. The neurodegenerative disease is any neurological disease that is associated with stacked proteins including, without limitation, transmissible spongiform encephalopathies such as Creutzfeldt-Jakob disease (CJD), multiple system atrophy (MSA), Alzheimer's disease, Parkinson's disease, Amyotrophic lateral sclerosis (ALS), Amyotrophic lateral sclerosis/Parkinsonism-dementia complex, anti-IgLON5-related tauopathy, Caribbean Parkinsonism, Chronic traumatic encephalopathy, Diffuse neurofibrillary tangles with calcification, Down syndrome, Familial British dementia, Familial Danish dementia, Niemann-Pick disease, type C, Non-Guamanian motor neuron disease with neurofibrillary tangles, Postencephalitic Parkinsonism, Primary age-related tauopathy, Progressive ataxia and palatal tremor, Tangle-only dementia, Familial frontotemporal dementia and Parkinsonism, Pick's disease, Argyrophilic grain disease, Corticobasal degeneration, Guadeloupean Parkinsonism, Globular glial tauopathy, Huntington's disease, Progressive supranuclear palsy, SLC9a-related Parkinsonism, Tau astrogliopathy, etc.

Alzheimer's disease (AD) is a progressive neurodegenerative disorder associated with memory loss, spatial disorientation, and gradual deterioration of intellectual capacity. Numerous pathological changes have been described in the postmortem brains of AD patients, including synaptic and neuronal loss, oxidative damage, activated inflammatory cells, amyloid plaques mainly composed of the ß-amyloid peptide (Aß), and neurofibrillary tangles (NFTs) comprised of hyperphosphorylated and/or acetylated aggregates of the microtubule-associated protein Tau, the latter two of which are considered the pathological hallmarks. For several reasons, research on the involvement of Aß in AD has progressed more quickly than that on Tau. The description of the “amyloid cascade hypothesis” based on the discovery of genetic mutations that cause autosomal familial AD centered the focus of research on Aß. Also, the biochemical studies of amyloid precursor protein (APP) and the presenilins have greatly enhanced the understanding of the molecular pathways leading to Aß generation. These studies favored the systematic development of disease-modifying therapies based on Aß pathway.

Tau is a soluble protein that normally binds to microtubules and regulates their dynamic growing and shortening behaviors. In Alzheimer's disease (AD) and other neurodegenerative diseases, Tau dissociates from microtubules and self-associates to form abnormal fibrillar aggregates. The distribution of these aberrant Tau structures is very well-correlated with neuronal cell death and the clinical progression of neuodegenerative diseases, suggesting an intimate link between aberrant Tau structure and neurodegeneration/dementia. Recent efforts to identify the neurotoxic species of Tau have shifted focus away from mature, fibrillar aggregates toward smaller oligomeric Tau species. Studies with antibodies that selectively recognize Tau oligomers have demonstrated that these species are elevated in AD brains. Cell to cell transmission of oligomeric Tau and other aberrant pre-fibrillar species may underlie the spread of Tau pathology, and these species show promise as potential therapeutic targets. A structural understanding of early Tau aggregates may lead to a deeper understanding of their neurotoxic mechanisms, as well as to the rational design of therapeutic drugs.

As a result of alternative RNA splicing there are 6 distinct isoforms of Tau that differ from one another depending upon the presence or absence of three inserts encoded by exons 2, 3 and 10 of the Tau gene. There are two important domains in each Tau isoform: the N-terminal projection domain determines the inter-microtubule spacing between bundled microtubules and also mediates interactions of microtubules with plasma membrane. Exons 2 and 3 each encode 29-residue acidic inserts located in the N-terminal projection domain. In contrast, the microtubule binding pseudo-repeat (MTBR) domain contains either three or four imperfect repeats (depending upon the presence or absence of exon 10 encoded sequences) and serves to bind microtubules directly and to regulate their dynamics. This same region of the protein makes up the core of fibrillar Tau aggregates, and Tau aggregation is accompanied by a regional transition from random coil to β-sheet structure. Fibrillar Tau aggregates have a cross-β-structure typical of amyloid fibrils. Finally, the 6 tau isoforms differ from one another not only structurally but also in terms of the relative expression levels and their rates and extents of fibril formation. Genetic evidence demonstrates unequivocally that functional differences must exist among the 6 different tau isoforms.

The amino acid sequence of the six human tau isoforms include the following:

isoform 1
(SEQ ID NO: 1)
MAEPRQEFEVMEDHAGTYGLGDRKDQGGYTMHQDQEGDTD
AGLKAEEAGIGDTPSLEDEAAGHVTQARMVSKSKDGTGSD
DKKAKGADGKTKIATPRGAAPPGQKGQANATRIPAKTPPA
PKTPPSSGEPPKSGDRSGYSSPGSPGTPGSRSRTPSLPTP
PTREPKKVAVVRTPPKSPSSAKSRLQTAPVPMPDLKNVKS
KIGSTENLKHQPGGGKVQIVYKPVDLSKVTSKCGSLGNIH
HKPGGGQVEVKSEKLDFKDRVQSKIGSLDNITHVPGGGNK
KIETHKLTFRENAKAKTDHGAEIVYKSPVVSGDTSPRHLS
NVSSTGSIDMVDSPQLATLADEVSASLAKQGL.
Isoform 2
(SEQ ID NO: 2):
MAEPRQEFEVMEDHAGTYGLGDRKDQGGYTMHQDQEGDTD
AGLKESPLQTPTEDGSEEPGSETSDAKSTPTAEAEEAGIG
DTPSLEDEAAGHVTQARMVSKSKDGTGSDDKKAKGADGKT
KIATPRGAAPPGQKGQANATRIPAKTPPAPKTPPSSGEPP
KSGDRSGYSSPGSPGTPGSRSRTPSLPTPPTREPKKVAVV
RTPPKSPSSAKSRLQTAPVPMPDLKNVKSKIGSTENLKHQ
PGGGKVQIVYKPVDLSKVTSKCGSLGNIHHKPGGGQVEVK
SEKLDFKDRVQSKIGSLDNITHVPGGGNKKIETHKLTFRE
NAKAKTDHGAEIVYKSPVVSGDTSPRHLSNVSSTGSIDMV
DSPQLATLADEVSASLAKQGL.
Isoform 3
(SEQ ID NO: 3):
MAEPRQEFEVMEDHAGTYGLGDRKDQGGYTMHQDQEGDTD
AGLKESPLQTPTEDGSEEPGSETSDAKSTPTAEDVTAPLV
DEGAPGKQAAAQPHTEIPEGTTAEEAGIGDTPSLEDEAAG
HVTQARMVSKSKDGTGSDDKKAKGADGKTKIATPRGAAPP
GQKGQANATRIPAKTPPAPKTPPSSGEPPKSGDRSGYSSP
GSPGTPGSRSRTPSLPTPPTREPKKVAVVRTPPKSPSSAK
SRLQTAPVPMPDLKNVKSKIGSTENLKHQPGGGKVQIVYK
PVDLSKVTSKCGSLGNIHHKPGGGQVEVKSEKLDFKDRVQ
SKIGSLDNITHVPGGGNKKIETHKLTFRENAKAKTDHGAE
IVYKSPVVSGDTSPRHLSNVSSTGSIDMVDSPQLATLADE
VSASLAKQGL
Isoform 4
(SEQ ID NO: 4):
MAEPRQEFEVMEDHAGTYGLGDRKDQGGYTMHQDQEGDTD
AGLKAEEAGIGDTPSLEDEAAGHVTQARMVSKSKDGTGSD
DKKAKGADGKTKIATPRGAAPPGQKGQANATRIPAKTPPA
PKTPPSSGEPPKSGDRSGYSSPGSPGTPGSRSRTPSLPTP
PTREPKKVAVVRTPPKSPSSAKSRLQTAPVPMPDLKNVKS
KIGSTENLKHQPGGGKVQIINKKLDLSNVQSKCGSKDNIK
HVPGGGSVQIVYKPVDLSKVTSKCGSLGNIHHKPGGGQVE
VKSEKLDFKDRVQSKIGSLDNITHVPGGGNKKIETHKLTF
RENAKAKTDHGAEIVYKSPVVSGDTSPRHLSNVSSTGSID
MVDSPQLATLADEVSASLAKQGL.
Isoform 5
(SEQ ID NO :5):
MAEPRQEFEVMEDHAGTYGLGDRKDQGGYTMHQDQEGDTD
AGLKESPLQTPTEDGSEEPGSETSDAKSTPTAEAEEAGIG
DTPSLEDEAAGHVTQARMVSKSKDGTGSDDKKAKGADGKT
KIATPRGAAPPGQKGQANATRIPAKTPPAPKTPPSSGEPP
KSGDRSGYSSPGSPGTPGSRSRTPSLPTPPTREPKKVAVV
RTPPKSPSSAKSRLQTAPVPMPDLKNVKSKIGSTENLKHQ
PGGGKVQIINKKLDLSNVQSKCGSKDNIKHVPGGGSVQIV
YKPVDLSKVTSKCGSLGNIHHKPGGGQVEVKSEKLDFKDR
VQSKIGSLDNITHVPGGGNKKIETHKLTFRENAKAKTDHG
AEIVYKSPVVSGDTSPRHLSNVSSTGSIDMVDSPQLATLA
DEVSASLAKQGL
Isoform 6
(SEQ ID NO: 6):
MAEPRQEFEVMEDHAGTYGLGDRKDQGGYTMHQDQEGDTD
AGLKESPLQTPTEDGSEEPGSETSDAKSTPTAEDVTAPLV
DEGAPGKQAAAQPHTEIPEGTTAEEAGIGDTPSLEDEAAG
HVTQARMVSKSKDGTGSDDKKAKGADGKTKIATPRGAAPP
GQKGQANATRIPAKTPPAPKTPPSSGEPPKSGDRSGYSSP
GSPGTPGSRSRTPSLPTPPTREPKKVAVVRTPPKSPSSAK
SRLQTAPVPMPDLKNVKSKIGSTENLKHQPGGGKVQIINK
KLDLSNVQSKCGSKDNIKHVPGGGSVQIVYKPVDLSKVTS
KCGSLGNIHHKPGGGQVEVKSEKLDFKDRVQSKIGSLDNI
THVPGGGNKKIETHKLTFRENAKAKTDHGAEIVYKSPVVS
GDTSPRHLSNVSSTGSIDMVDSPQLATLADEVSASLAKQG
L.

When specific amino acid numbers are referenced, the numbering refers to SEQ ID NO: 6 unless specifically indicated otherwise.

The classically described function of Tau is as a neuronal microtubule-associated protein, mainly found in axons. Under physiological conditions, Tau exists as a highly soluble and natively unfolded protein that interacts with tubulin and promotes its assembly into microtubules, which helps to stabilize their structure. Recent evidence points to additional functions for Tau. For example, Tau phosphorylation enables neurons to escape from an acute apoptotic death through stabilizing ß-catenin. Also, Tau exerts an essential role in the balance of microtubule-dependent axonal transport of organelles and biomolecules by modulating the anterograde transport by kinesin and the dynein-driven retrograde transport.

Soluble oligomeric species of amyloid-β (Aβ) are thought to be other key mediators of cognitive dysfunction in Alzheimer's disease (AD) (M. Sheng, et al. (2012) Cold Spring Harb Perspect Biol 4; J. J. Palop et al. (2010) Nat Neurosci 13, 812). Neuritic plaques, a hallmark of Alzheimer's Disease, are accumulations of aggregated, or oligomerized, amyloid beta (Aβ) peptides, including Aβ1-40 (Aβ40) and Aβ1-42 (Aβ42) that are derived from the processing of amyloid precursor protein (APP) by β- and γ-secretases. The vast majority of autosomal familial AD (FAD)-linked mutations are associated with increased levels of Aβ1-42. Transgenic mice expressing elevated levels of human Aβ experience memory loss and synaptic regression (M. Faizi et al., (2012) Brain Behav 2, 142; C. Perez-Cruz et al., (2011) J Neurosci 31, 3926; S. Knafo et al., (2009) Cereb Cortex 19, 586; M. Cisse et al., (2011) Nature 469, 47). Aβ production is thought to be activity-dependent (F. Kamenetz et al., (2003) Neuron 37, 925; J. Wu et al., (2011) Cell 147, 615), and even in wild type mice addition of soluble Aβ oligomers to hippocampal slices or cultures induces loss of long-term 2 potentiation (LTP), increases long-term depression (LTD) and decreases dendritic spine density (G. M. Shankar et al., (2007) J Neurosci 27, 2866; G. M. Shankar et al., (2008) Nat Med 14, 837; H. Hsieh et al., (2006) Neuron 52, 831). There are currently no effective therapies for arresting or reversing the impairment of cognitive function that characterizes AD.

Aβ oligomer levels are also elevated by about 200-300% in Down syndrome (DS) patients throughout life (reviewed in Head and Lott (2004) Curr Opin Neurol 17(2):95-100). The use of a γ-secretase inhibitor to lower β-amyloid levels in young mice that model DS corrected learning deficits characteristic of these mice, suggesting that therapies that interfere with Aβ oligomers will improve cognitive function in young DS patients as well (Netzer W J, et al. (2010) PLoS One 5:e10943).

By Aβ, or “amyloid beta”, or “amyloid β”, it is meant a peptide of 36-43 amino acids that is derived from the processing of amyloid precursor protein (APP) by β- and γ-secretases. By “Aβ oligomers”, “amyloid β oligomers”, or “amyloid beta oligomers” it is meant aggregates of Aβ peptide. Aβ is the main component of deposits, called amyloid plaques, found in the brains of patients with Alzheimer's disease (AD) and cerebral amyloid angiopathy (CAA); it also associated with retinal ganglion cells in patients having glaucoma. Two major variants, Aβ₁₋₄₀ (“Aβ40”) (DAEFRHDSGYEVHHQKLVFFAEDVGSNKGAIIGLMVGGVV) (SEQ ID NO: 7) and Aβ₁₋₄₂ (“Aβ42”) (DAEFRHDSGYEVHHQKLVFAAEDVGSNKGAIIGLMVGGVVIA) (SEQ ID NO: 8), are produced by alternative carboxy-terminal truncation of APP (Selkoe et al. (1988) Proc. Natl. Acad. Sci. USA 85:7341-7345; Selkoe, (1993) Trends Neurosci 16:403-409). Aβ₁₋₄₂ is the more fibrillogenic and more abundant of the two peptides in amyloid deposits of both AD and CAA. Derivatives of the above peptides comprising a naturally occurring substitution, e.g. Aβ₁₋₄₂ H13R, Aβ₁₋₄₂ V18A, Aβ₁₋₄₂ F19P, Aβ₁₋₄₂ E22D, Aβ₁₋₄₂ E22V, Aβ₁₋₄₂ E22A, Aβ₁₋₄₂ D23A, Aβ₁₋₄₂ G25A, Aβ₁₋₄₂ N27A, Aβ₁₋₄₂ K28A, Aβ₁₋₄₂ G29A, Aβ₁₋₄₂ I31A, Aβ₁₋₄₂ G37A, the English Mutation, the Iowa Mutation, the Tottori-Japanese Mutation, the Flemish Mutation, the Arctic Mutation, the Italian Mutation, etc. In addition to the amyloid deposits that may occur in, for example, CNS tissue, amyloid deposition may occur in the vascular walls (Hardy (1997); Haan et al. (1990); Vinters (1987); Itoh et al. (1993); Yamada et al. (1993); Greenberg et al. (1993); Levy et al. (1990)). These vascular lesions are the hallmark of CAA, which can exist in the absence of AD.

Aβ oligomers are known in the art to have a number of effects on cells. These include, for example, reducing cell viability, and reducing synaptic plasticity, promoting synapse loss in neurons. By a “synapse” it is meant the structure on a neuron that permits the neuron to pass an electrical or chemical signal to another cell. By “synaptic plasticity” it is meant the ability of the synapse to change in strength, i.e. to become stronger or weaker, in response to either use or disuse, respectively, of transmission over that synaptic pathway. Such a change in strength is typically evident by one or more of the following structural changes: a change in the number of presynaptic vesicles, a change in the amount of neurotransmitter loaded per vesicle, a change in the number of dendritic spines, and/or a change in the number of neurotransmitter receptors positioned on the postsynaptic neuron. Reductions or enhancements in synaptic plasticity may be observed by assessing the ability of a postsynaptic neuron to evoke a long-term enhancement (“long term potentiation”, LTP) or long-term depression (LTD) in the activity of a presynaptic neuron, and/or by assaying for the subsequent changes in synaptic strength, e.g. by detecting one or more of the above-mentioned structural changes. By “enhanced synaptic plasticity” it is meant greater synaptic strengthening (LTP), more stable synapses and a failure to remove synapses and the spines that carry synapses. By “reduced synaptic plasticity” it is meant enhanced synaptic weakening (LTD), less stable synapses, and fewer spines and synapses. By “synapse loss” it is meant a decrease in the number of synapses, for example, a loss in the connection between two neurons or, in instances in which multiple synapses exist between two neurons, in the loss of one or more of these synapses. As is well known in the art, synaptic activity and the change in the strength and number of synapses is central to almost all neurobiological processes, including learning, memory, and neuronal development. In further describing aspects of the invention, the following description focuses on the effects of Aβ oligomers on neurons. However, the subject methods and compositions also find use in inhibiting the effects of Aβ oligomers on other types of cells as well, for example, microglia.

Parkinson's disease (PD) is a major neurodegenerative disease that primarily affects motor systems but can also be accompanied by cognitive and behavioral problems. There is a widespread neuron degeneration in PD brains, affecting up to 70% of dopaminergic neurons in the substantia nigra (SN) by the time of death. The neuropathological hallmarks of PD include Lewy bodies (LB s) in the SN, brainstem, and rostral and forebrain regions and the selective deletion of dopaminergic neurons in the SN. Cell-death induced damage in SN may be the source of patient movement disorders. Although the causes of this cell death are generally unclear, researchers have observed an enrichment alpha-synuclein in neuronal Lewy bodies. Tau aggregates can also be observed in PD, for example in cases with LKKR2 mutations. Tau has also been associated with increased alpha synuclein deposits. Immunohistochemistry with anti-tau antibodies showed high level of NFTs in the substantia nigra from post-mortem human brain tissue. Researchers have also reported that tauopathies in PD and PD with dementia (PDD) were only observed in DA neurons of the nigrostriatal region, which contrasts with the wide-spread expression pattern of tau throughout the entire brain in AD.

In Parkinson disease, pigmented neurons of the substantia nigra, locus ceruleus, and other brain stem dopaminergic cell groups degenerate. Loss of substantia nigra neurons results in depletion of dopamine in the dorsal aspect of the putamen (part of the basal ganglia) and causes many of the motor manifestations of Parkinson disease.

A genetic predisposition is likely in at least in some cases of Parkinson disease. A genetic association with polymorphisms surrounding the tau gene is found in Parkinson disease and Alzheimer's dementia. About 10% of PD patients have a family history of Parkinson disease. Several abnormal genes have been identified. Inheritance is autosomal dominant for some genes and autosomal recessive for others. Mutations in leucine-rich repeat kinase 2 (LRRK2) are the most prevalent mutation in sporadic cases of Parkinson disease in patients, and it is the most prevalent autosomal dominant mutation of the inherited forms of the disease. Recent data shows that tau is particularly important in PD patient with LRRK2 mutation, thus the therapy proposed here could be particularly effective.

Diagnosis of Parkinson disease is clinical. Parkinson disease is suspected in patients with characteristic unilateral resting tremor, decreased movement, or rigidity. During finger-to-nose coordination testing, the tremor disappears (or attenuates) in the limb being tested. During the neurologic examination, patients cannot perform rapidly alternating or rapid successive movements well. Sensation and strength are usually normal. Reflexes are normal but may be difficult to elicit because of marked tremor or rigidity. Slowed and decreased movement due to Parkinson disease must be differentiated from decreased movement and spasticity due to lesions of the corticospinal tracts. To help distinguish Parkinson disease from secondary or atypical parkinsonism, clinicians often test responsiveness to levodopa. A large, sustained response strongly supports Parkinson disease.

Amyotrophic lateral sclerosis is a group of rare neurological diseases that mainly involve the nerve cells (neurons) responsible for controlling voluntary muscle movement. It is characterized by steady, relentless, progressive degeneration of corticospinal tracts, anterior horn cells, bulbar motor nuclei, or a combination. Symptoms vary in severity and may include muscle weakness and atrophy, fasciculations, emotional lability, and respiratory muscle weakness. Diagnosis involves nerve conduction studies, electromyography, and exclusion of other disorders via MRI and laboratory tests. Current treatment is supportive. The majority of ALS cases (90 percent or more) are considered sporadic.

Most patients with ALS present with random, asymmetric symptoms, consisting of cramps, weakness, and muscle atrophy of the hands (most commonly) or feet. Weakness progresses to the forearms, shoulders, and lower limbs. Fasciculations, spasticity, hyperactive deep tendon reflexes, extensor plantar reflexes, clumsiness, stiffness of movement, weight loss, fatigue, and difficulty controlling facial expression and tongue movements soon follow. Other symptoms include hoarseness, dysphagia, and slurred speech; because swallowing is difficult, salivation appears to increase, and patients tend to choke on liquids. Late in the disorder, a pseudobulbar affect occurs, with inappropriate, involuntary, and uncontrollable excesses of laughter or crying. Sensory systems, consciousness, cognition, voluntary eye movements, sexual function, and urinary and anal sphincters are usually spared. Death is usually caused by failure of the respiratory muscles; 50% of patients die within 3 yr of onset, 20% live 5 yr, and 10% live 10 yr. Survival for >30 yr is rare.

MSA is a sporadic synucleinopathy of adult onset, with symptoms of parkinsonism, cerebellar ataxia and autonomic failure. Cases of MSA are classified as MSA-P, which show predominant parkinsonism caused by striatonigral degeneration, and MSA-C, which show cerebellar ataxia associated with olivopontocerebellar atrophy. Autonomic dysfunction is common to both subtypes. In neuropathological terms, MSA is defined by regional nerve cell loss and the presence of abundant filamentous α-synuclein inclusions in oligodendrocytes: glial cytoplasmic inclusions, known as Papp-Lantos bodies. Smaller numbers of α-synuclein inclusions are also present in nerve cells. The mean duration of the disease is 6-10 years, but survival times of 18-20 years have been reported. The late appearance of autonomic dysfunction correlates with prolonged survival.

Synucleinopathies are defined pathologically by the presence of α-Syn (α-synuclein or alpha synuclein) aggregates. Lewy body diseases consist of synucleinopathies with pathologic Lewy bodies and Lewy neurites which clinically manifest as PD, Parkinson disease dementia (PDD), or dementia with Lewy bodies (DLB). α-Syn aggregates in the form of glial cytoplasmic inclusions (GCIs) are characteristic of MSA. It has been shown previously that fibrils of α-Syn exist in PD and are expected to exist in other synucleinopathies. α-Syn fibrils formed in vitro also induce α-Syn inclusions when injected into model animals.

α-Syn is a 140 amino acid protein encoded by the gene synuclein alpha (SNCA) whose normal function is thought to be related to synaptic vesicle transmission. Residues 1-60 compose a lysine-rich N-terminal region with KTK lipid-binding repeats for vesicle binding. Familial SNCA gene mutations are found in this N-terminal region, including: A30P, A30G, E46K, G51D, A53E, A53V, A53T. Residues 61-95 comprise the non-amyloid b component (NAC) region that has been shown as essential for aggregation. Small peptides derived from this region were easily able to aggregate. Furthermore, b-synuclein, which is similar to α-Syn but lacks amino acids 71-82 in the NAC, has not been found to aggregate like α-Syn. Removing this region from α-Syn also prevents aggregation in vitro further supporting the importance of this region in aggregation. The amino acid sequence of α-Syn is

(SEQ ID NO: 9)
MDVFMKGLSKAKEGVVAAAEKTKQGVAEAAGKTKEGVLYV
GSKTKEGVVHGVATVAEKTKEQVTNVGGAVVTGVTAVAQK
TVEGAGSIAAATGFVKKDQLGKNEEGAPQEGILEDMPVDP
DNEAYEMPSEEGYQDYEPEA

Clinically, PD is the second most common neurodegenerative disorder affecting 2%-3% individuals 65 and older, while DLB is thought to be the underlying cause of 10%-15% of all cases of dementia. PD is characterized by neuronal loss in the substantia nigra that causes striatal dopamine deficiency and leads to bradykinesia and other motor symptoms. PD can, however, progress to PDD, and DLB can progress to motor dysfunction similar to that seen in PD. PD, PDD, and DLB are generally considered to be on the same disease spectrum with similarities clinically and pathologically including α-Syn aggregates in the form of Lewy bodies and Lewy neurites. MSA is distinct clinically and pathologically from PD and DLB, with a lower prevalence in the general population. MSA is of sporadic onset and is characterized by parkinsonism, cerebellar ataxia, and/or autonomic failure. Similar to PD and DLB there are neuronal asyn inclusions and neuronal cell loss in MSA, however, α-Syn inclusions are more prevalent in oligodendrocytes as GCIs.

As is seen with tau fibrils, the structure of α-Syn fibrils varies among disease states. It was also shown that GCIs from MSA are 103-times more potent seeds of aggregation than fibrils derived from PD Lewy bodies. Recent characterization of amplified patient-derived fibrils suggests that PD and MSA fibrils share many characteristics, but MSA fibrils are more potent inducers of motor deficits, neurodegeneration, and α-Syn pathology. Although DLB and PD are thought to be on the same disease spectrum, DLB fibrils did not yield the significant neuropathology seen with PD fibrils. Though still recent, these results may suggest that PD fibrils and DLB fibrils contained in Lewy bodies may be less similar than previously thought.

Familial SNCA mutations disrupt the stabilizing interactions of the known wild-type α-Syn in vitro fold conformations. It has been shown that under the same conditions A53T, A53E, G51D, and E46K mutants of α-Syn form fibrils with distinct morphologies from wild-type fibrils. The E46K mutant α-Syn is toxic to neuronal cells. These fibrils were less stable and easily fragmented, but were also a better seed than wild-type fibrils, potentially indicating that toxicity is related to seeding rather than fibrils stability. These in vitro data suggest that mutations may affect fibril structure, stability, and effect on disease progression.

G51D and A53E mutations can cause mixed MSA and PD pathology in patients. G51 lies at the protofilament interface of the ex vivo MSA fibrils near K43, K45, and H50. Structurally, there may be room to accommodate the switch from glycine to aspartic acid; however, this switch would reduce the positive charge of the central cavity. It is difficult to speculate how a change in charge or amino acid side chain would affect the central cavity and the in vivo fibrils as a whole. However, it could change the nonproteinaceous compounds within the central cavity or potentially disrupt the fibril stability or even enhance fibril stability to make fibrils more stable and more pathogenic than wild-type α-Syn fibrils leading to mixed MSA and PD pathology.

Some of the in vitro fibrils imaged may be able to explain how some of the familial mutations pack in vivo and the ex vivo structures solved may be able to accommodate some of the familial SNCA mutations. However, additional ex vivo α-Syn structures of mutant fibrils will need to be solved in order to confirm the actual structure. The structure of fibrils from patients with familial mutations will be important to understanding why those mutations specifically cause disease and potentially lead to breakthroughs in understanding development and progression of sporadic disease as well.

### Compounds

The present disclosure provides novel compounds. The novel compounds, as well as pharmaceutical formulations containing such compounds or combinations of these compounds with at least one additional therapeutic agent, can be used for, among other things, treating diseases described herein, such as multiple system atrophy (MSA).

The symbol [symbol] , whether utilized as a bond or displayed perpendicular to a bond, indicates the point at which the displayed moiety is attached to the remainder of the compound.

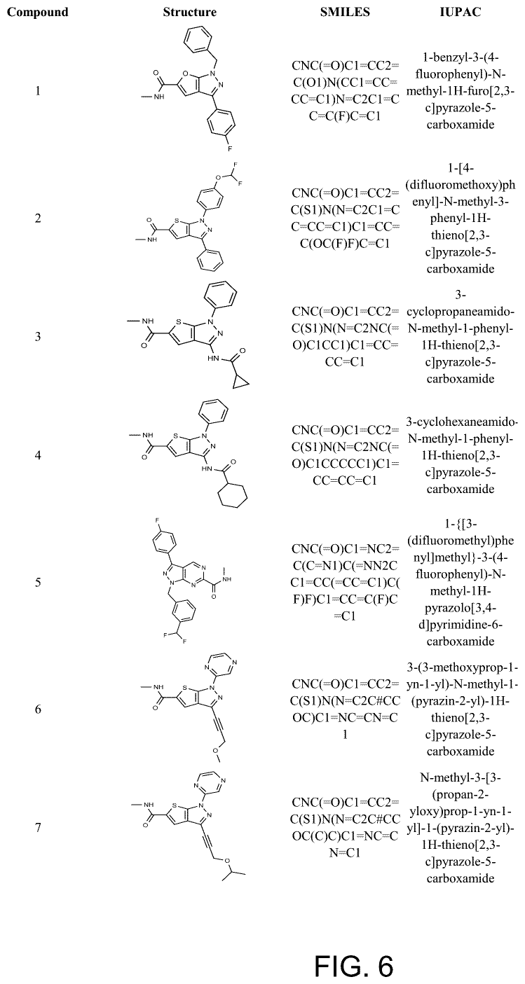

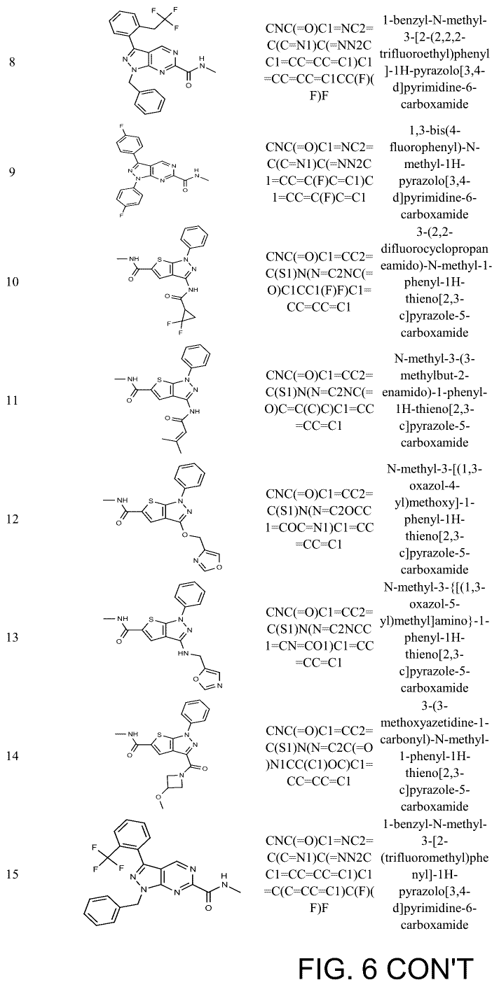

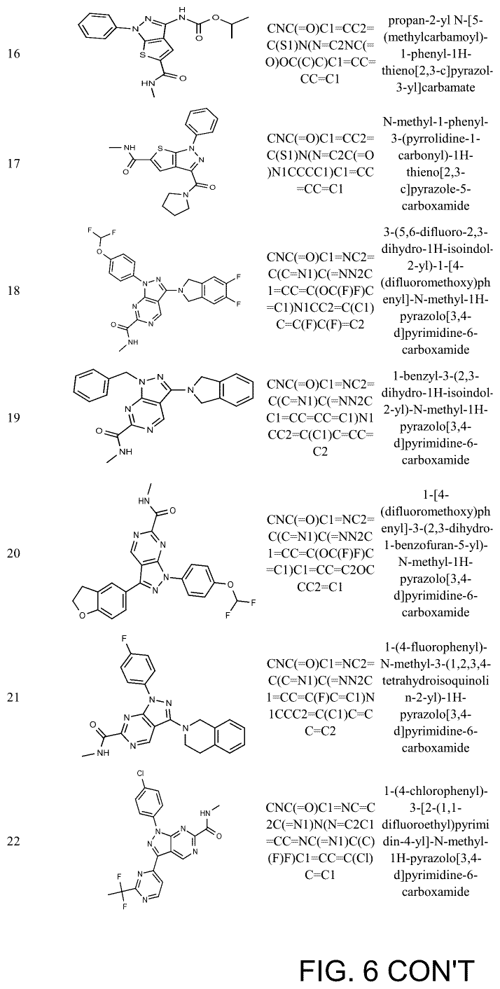

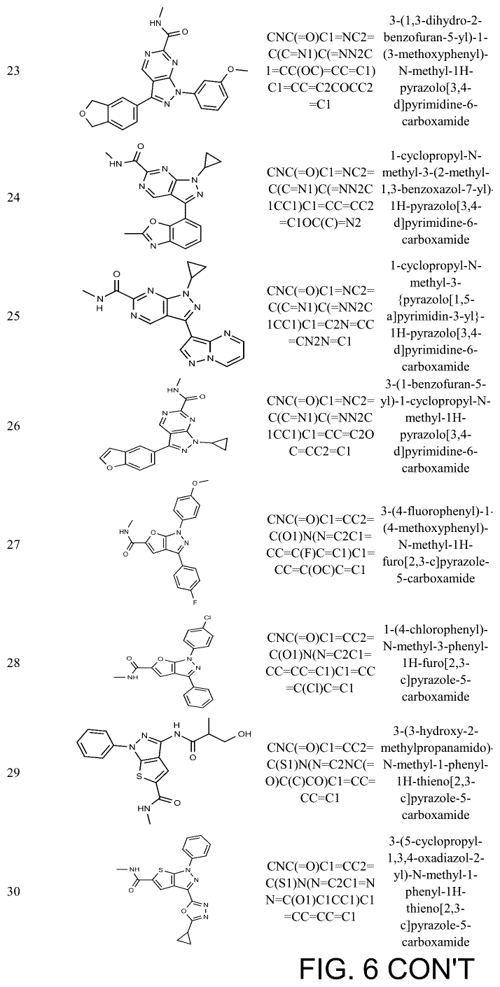

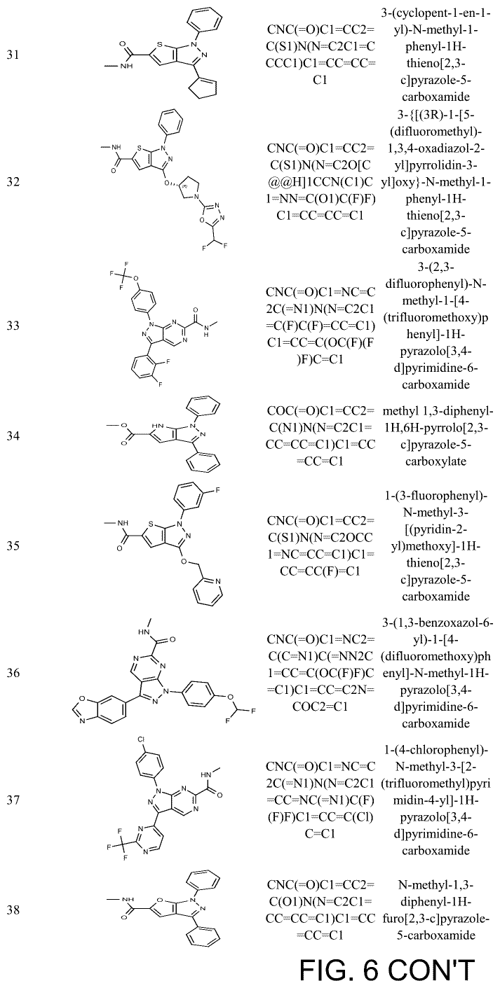

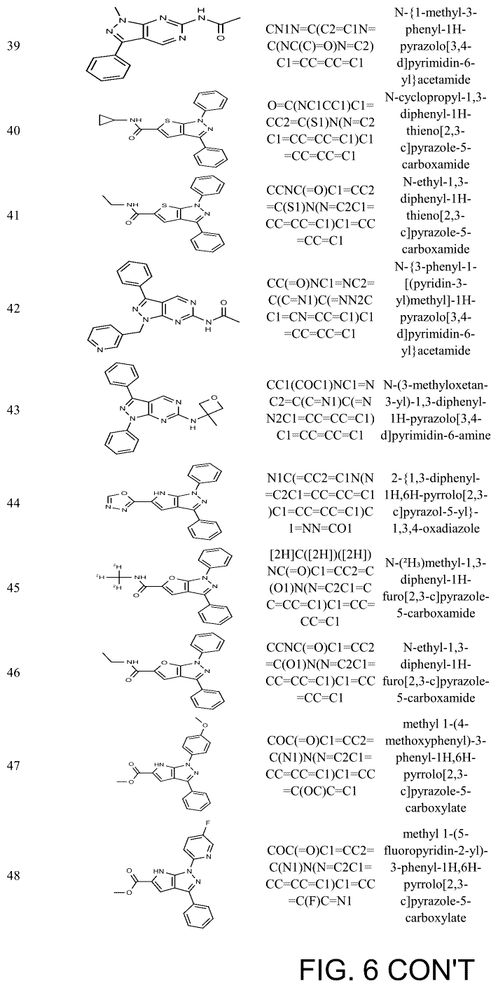

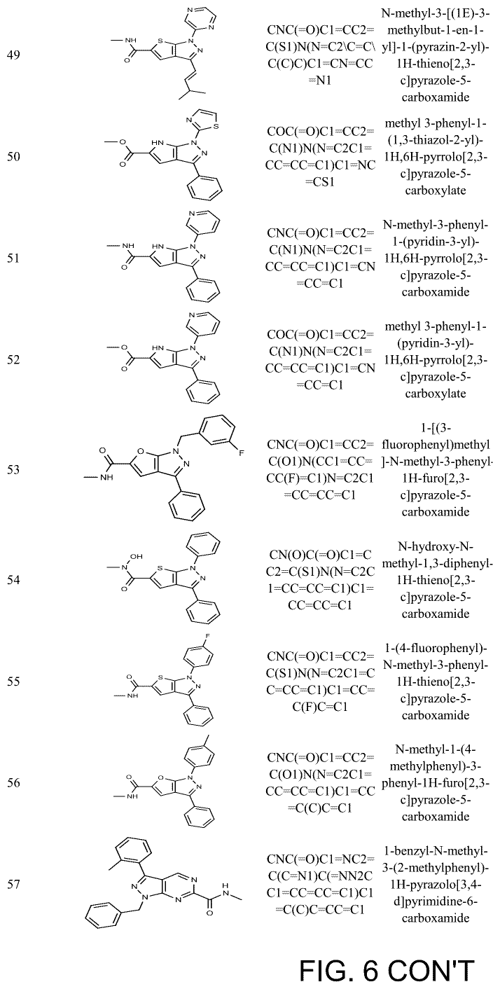

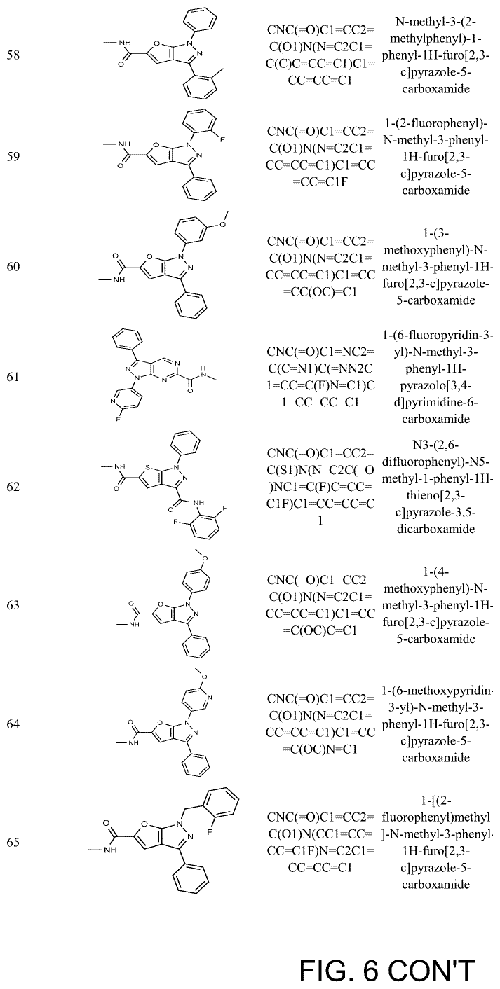

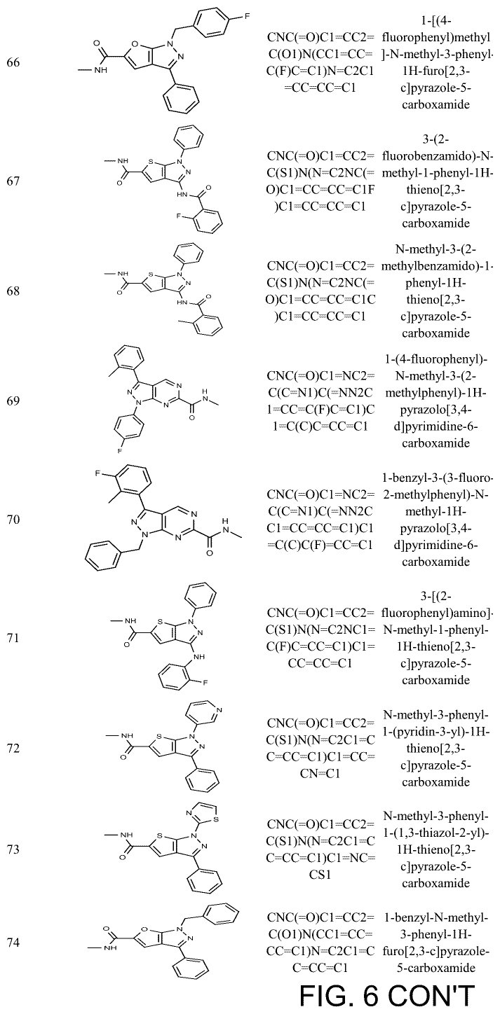

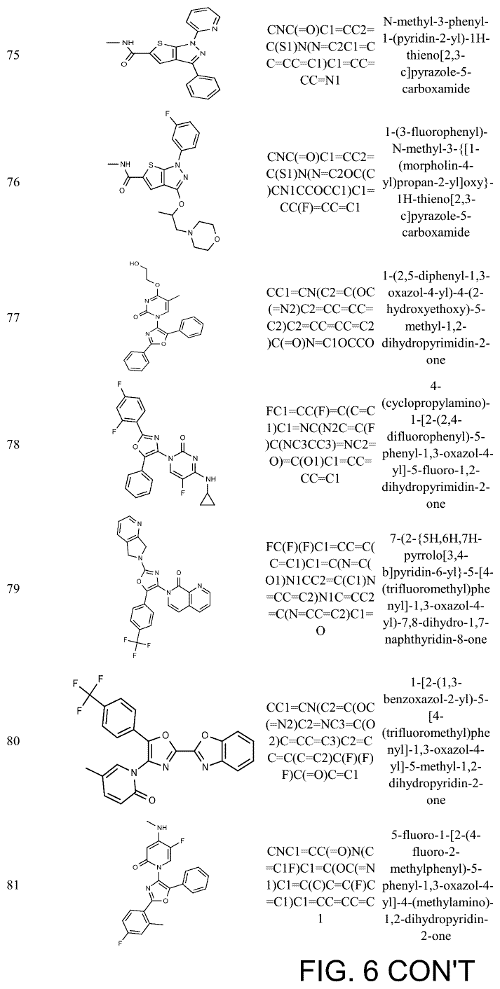

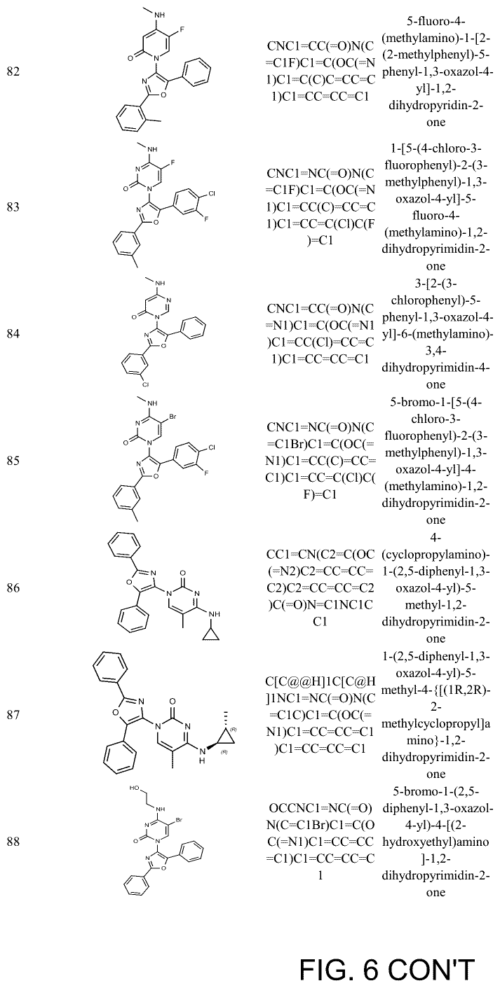

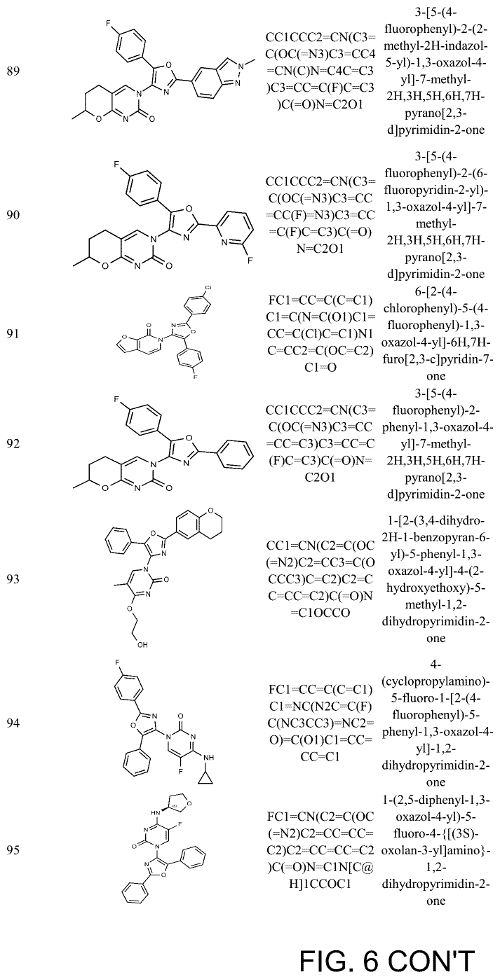

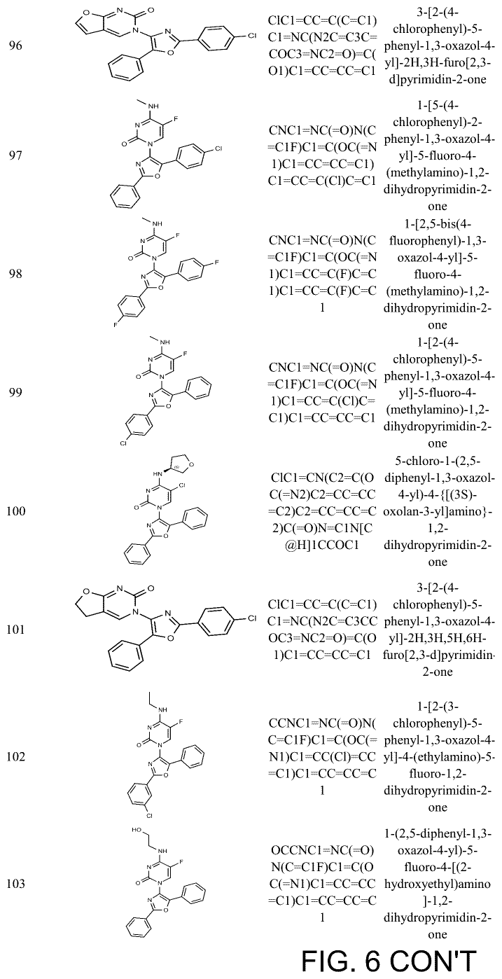

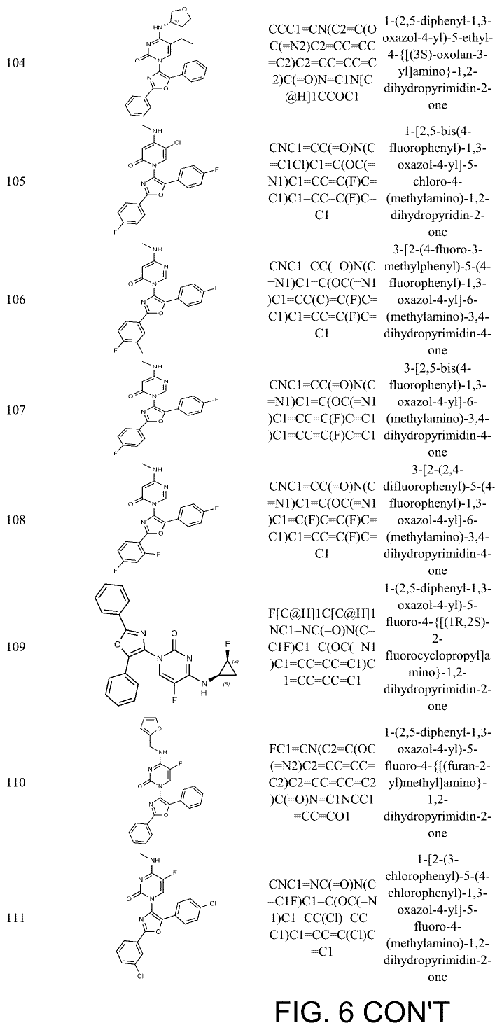

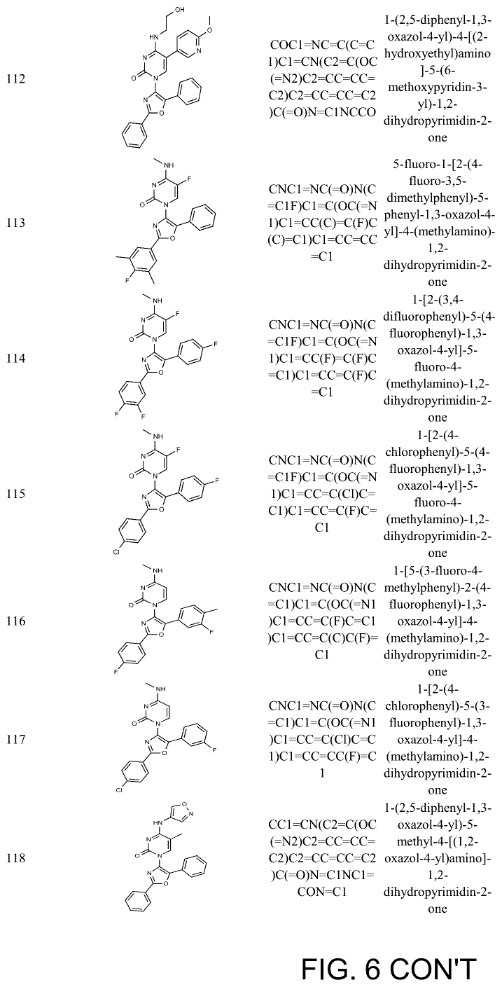

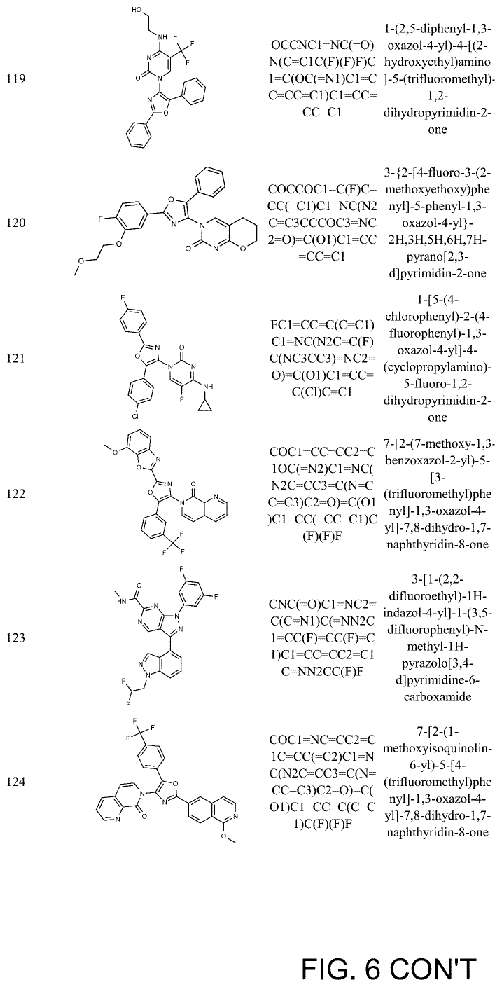

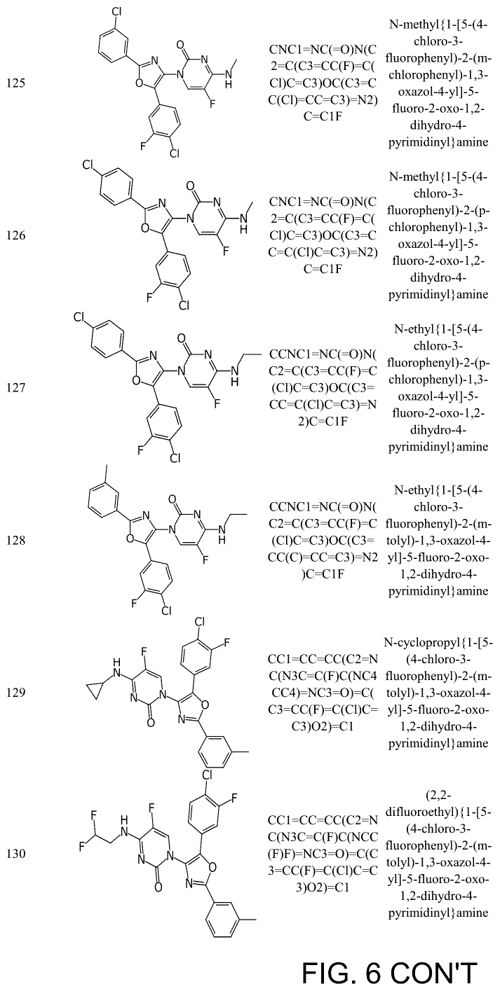

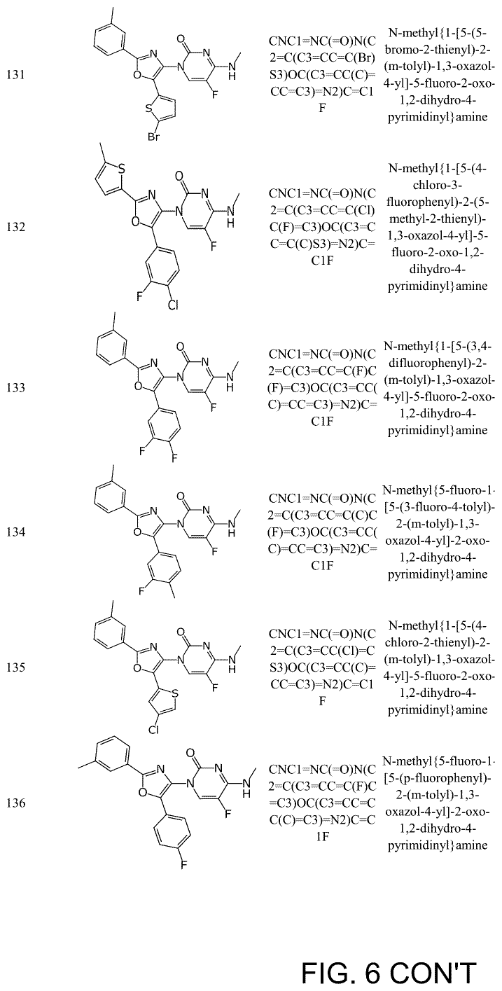

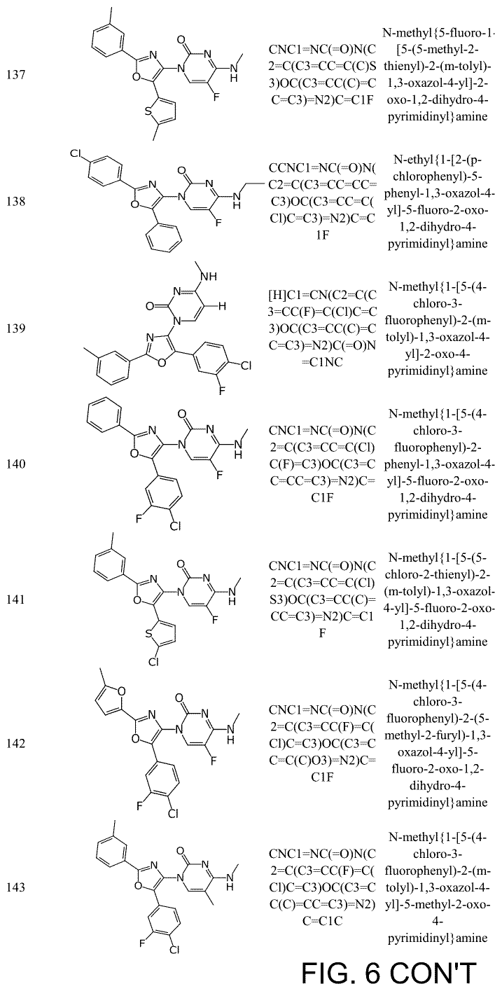

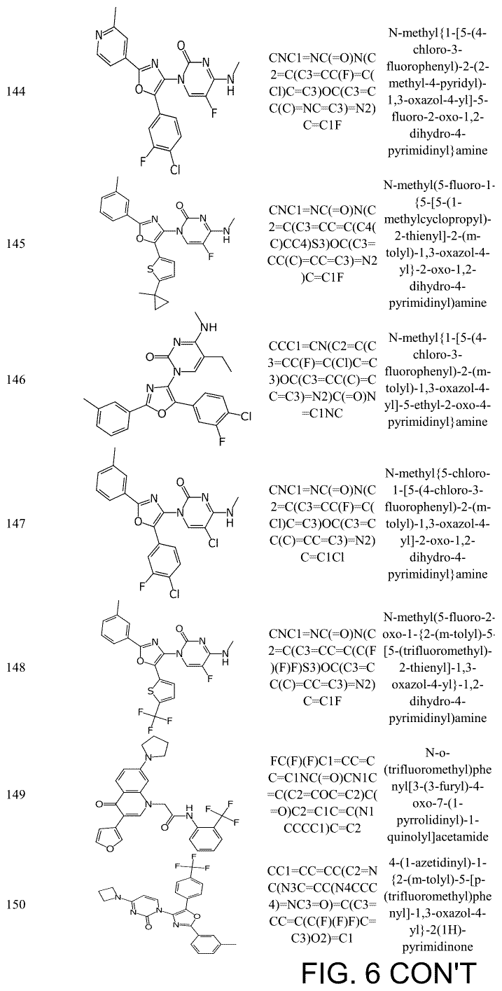

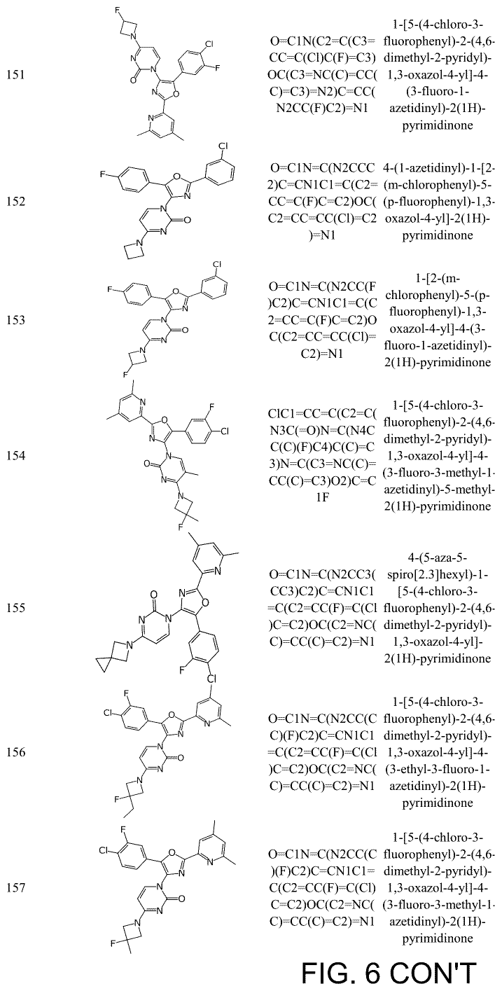

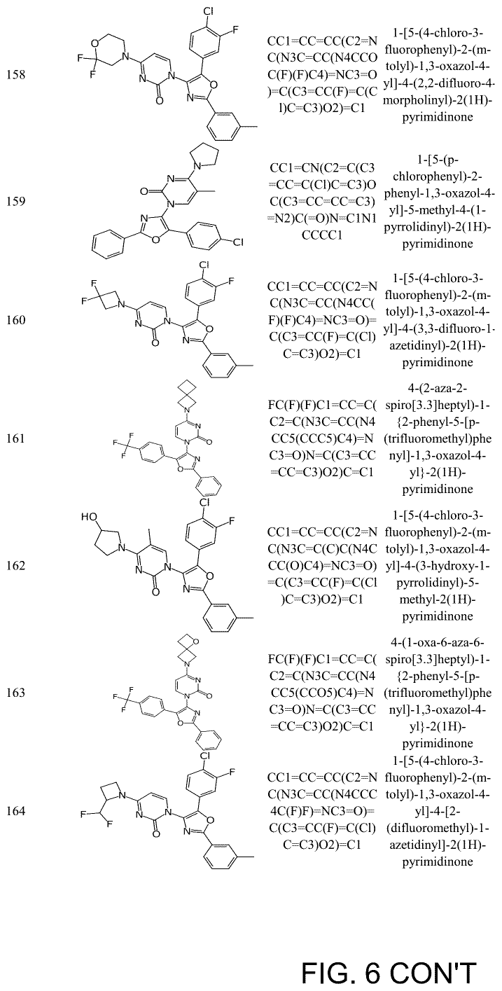

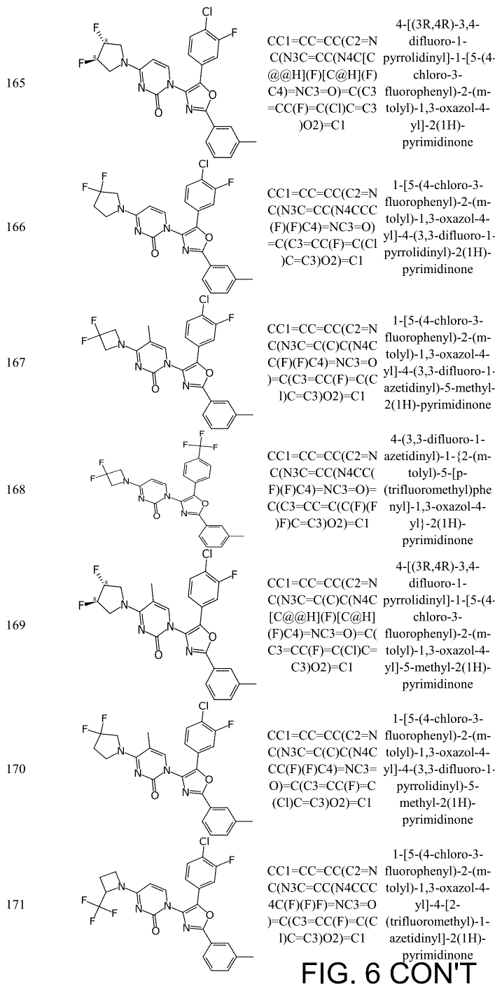

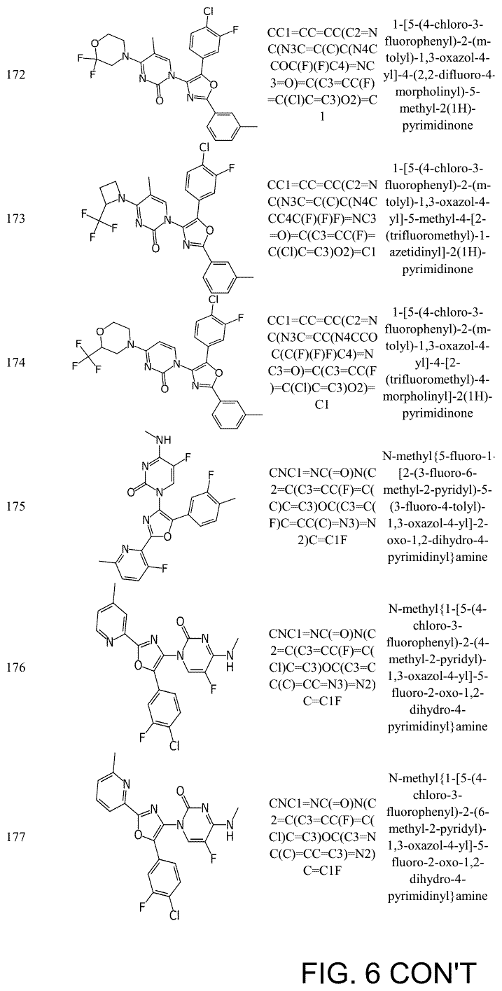

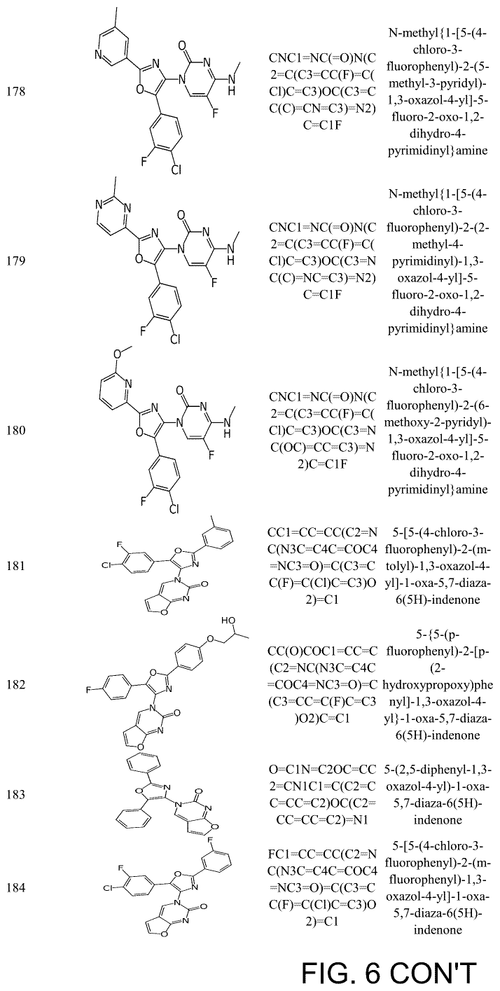

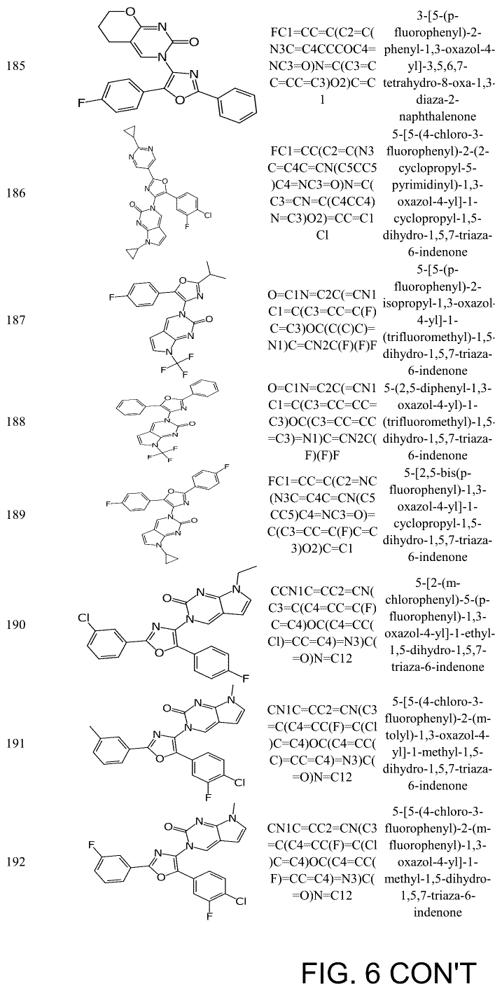

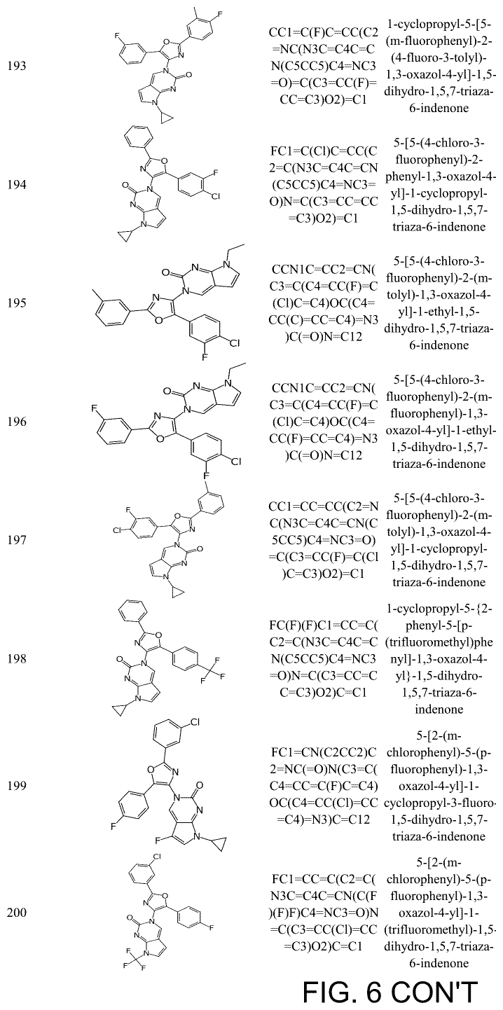

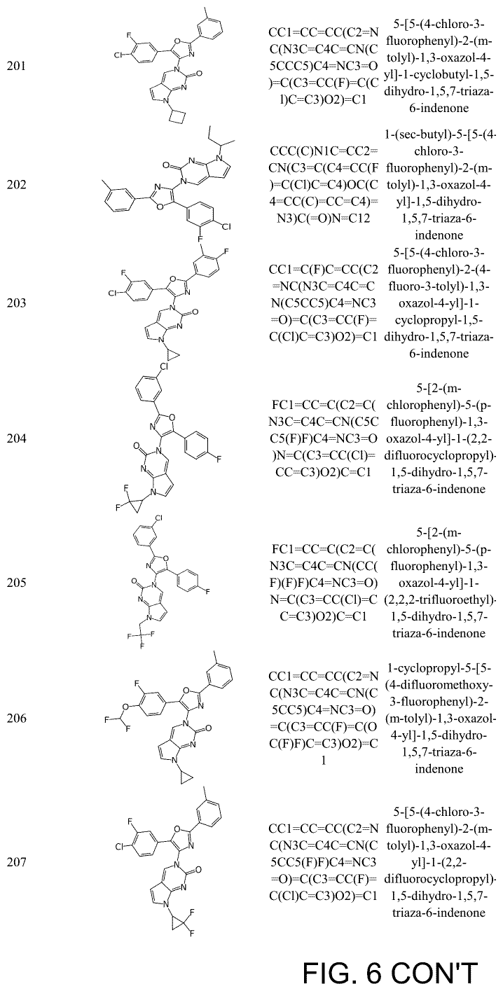

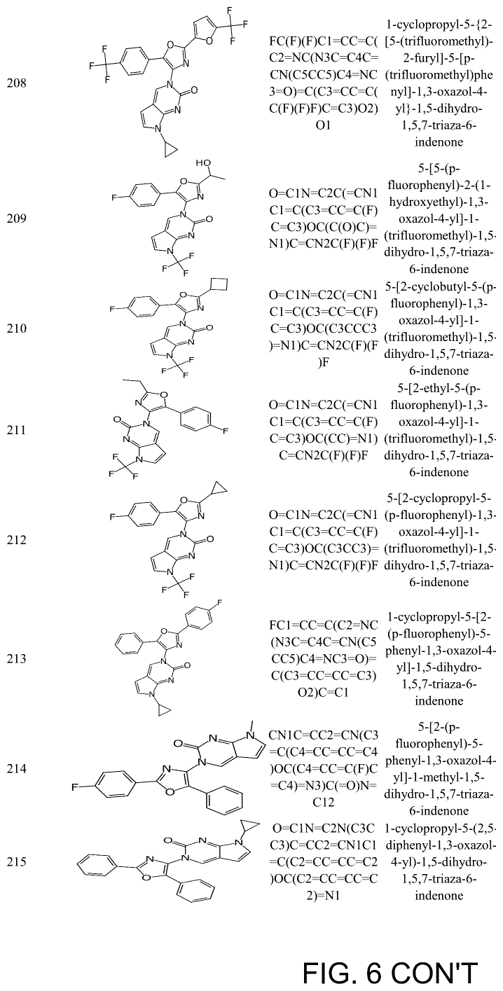

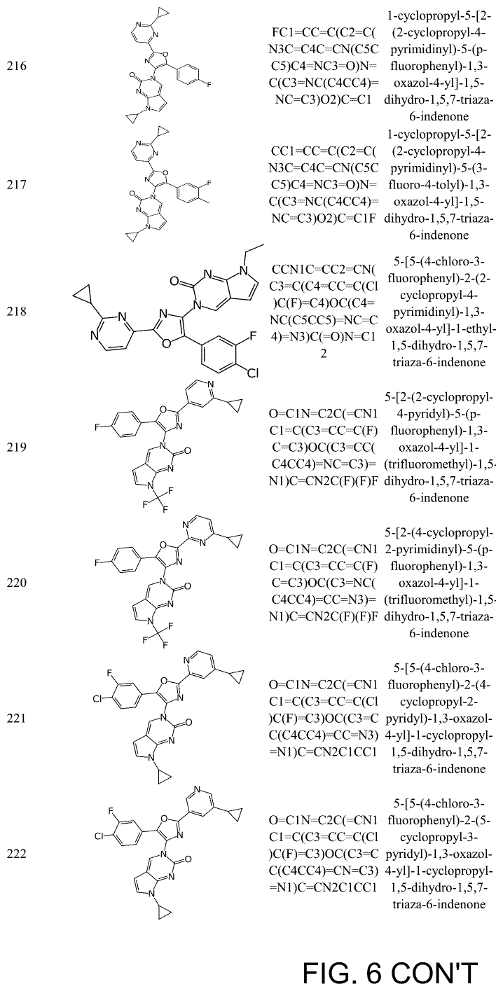

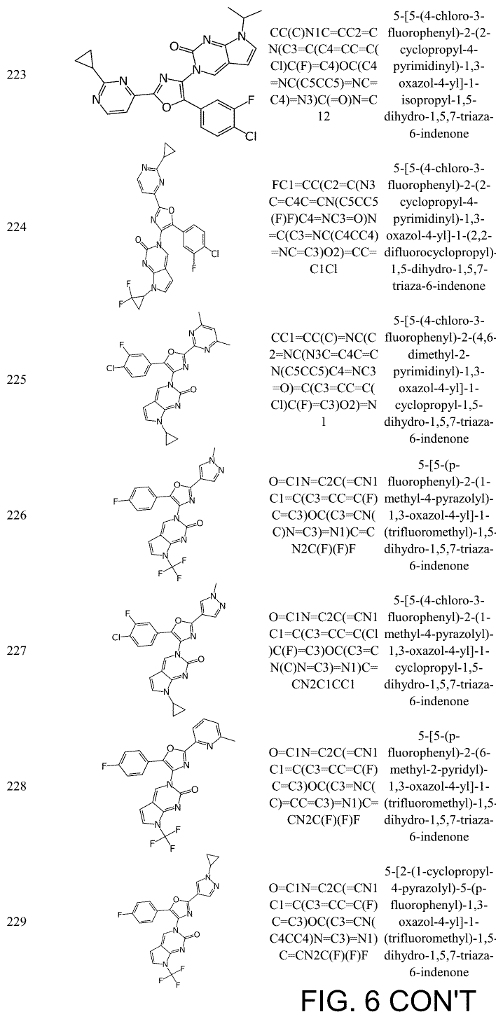

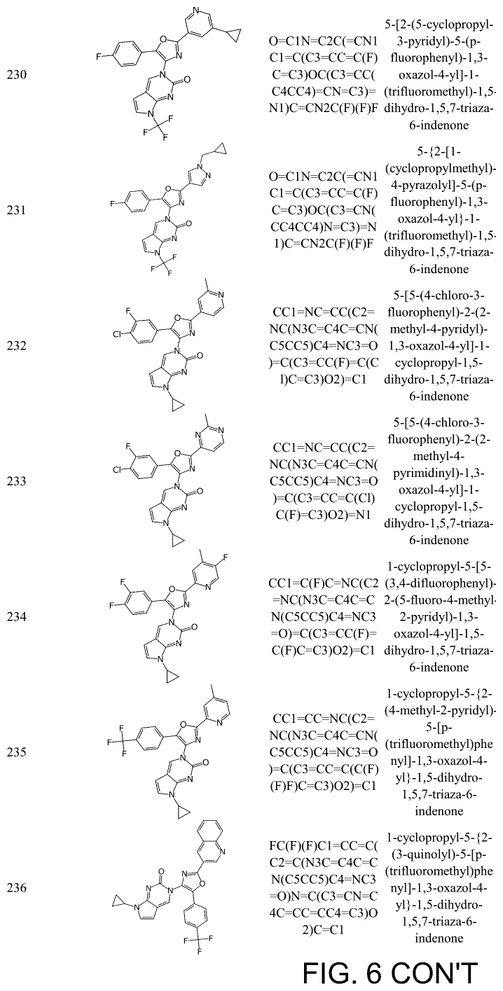

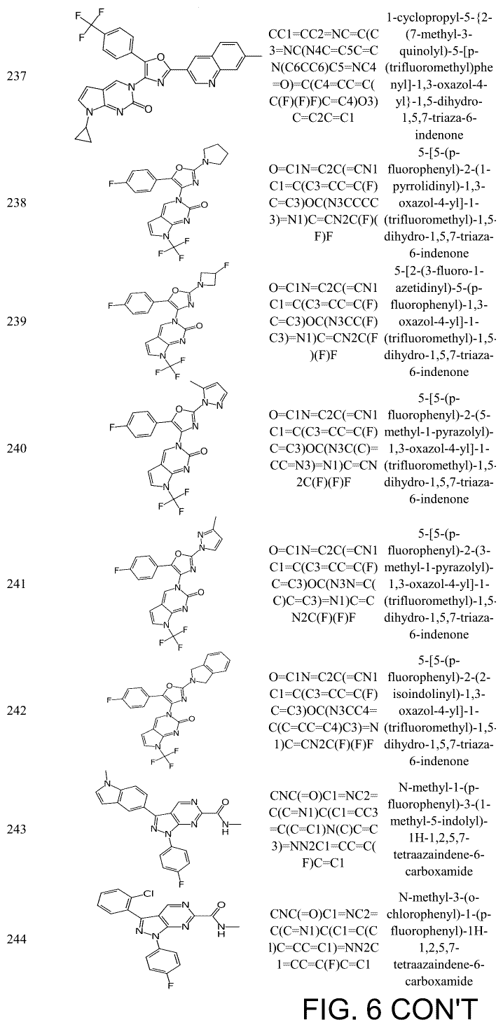

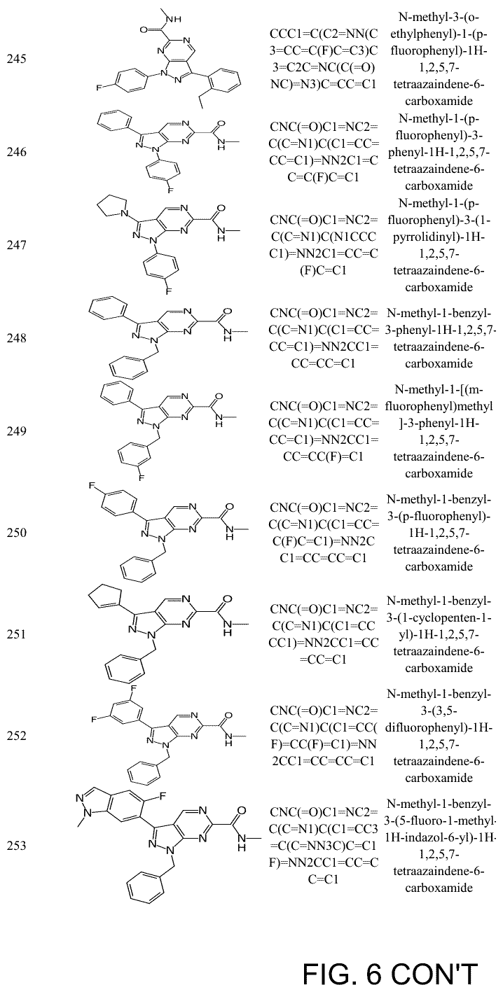

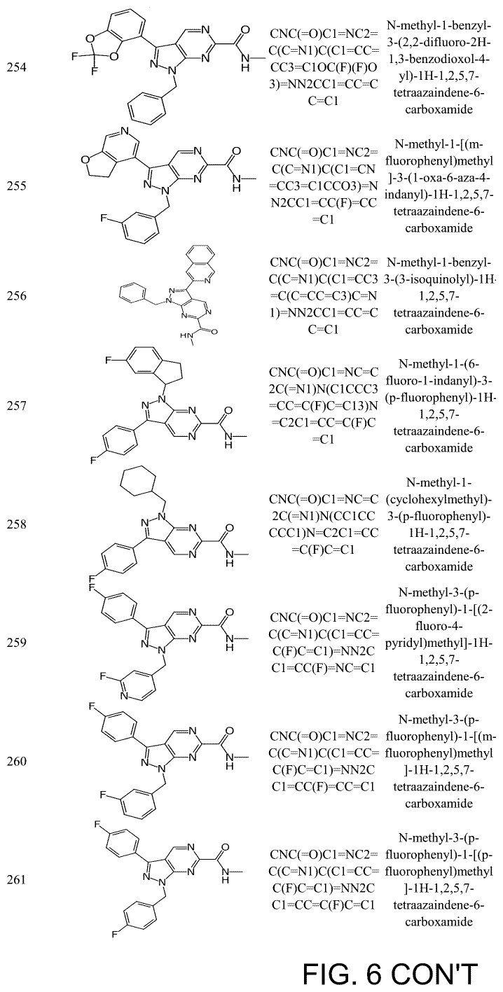

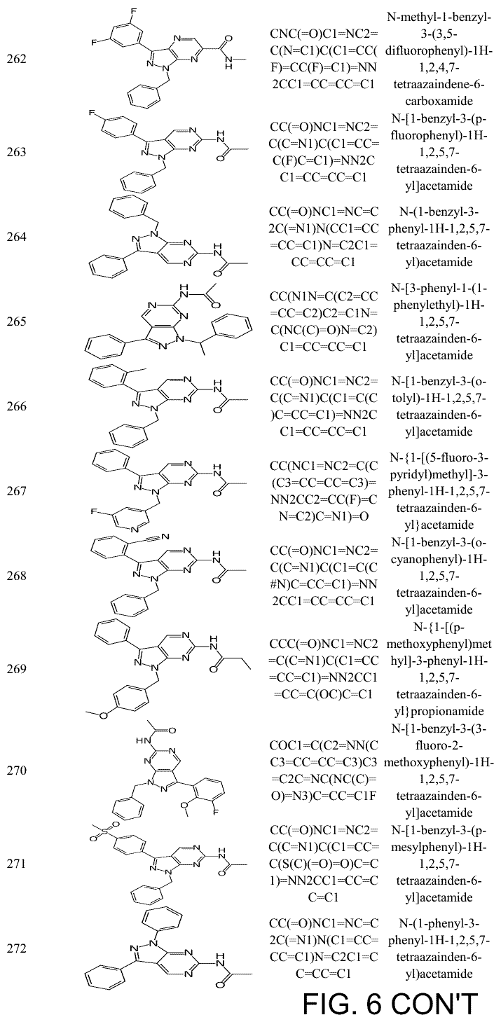

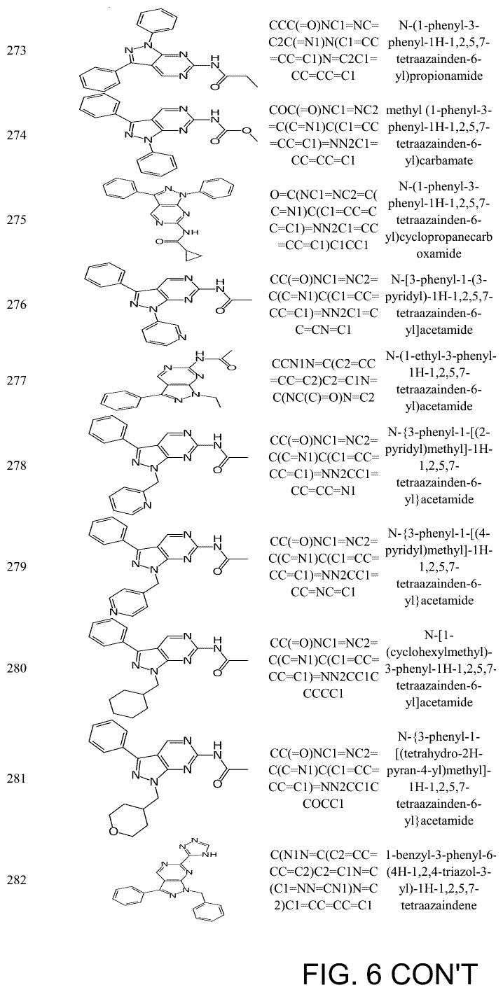

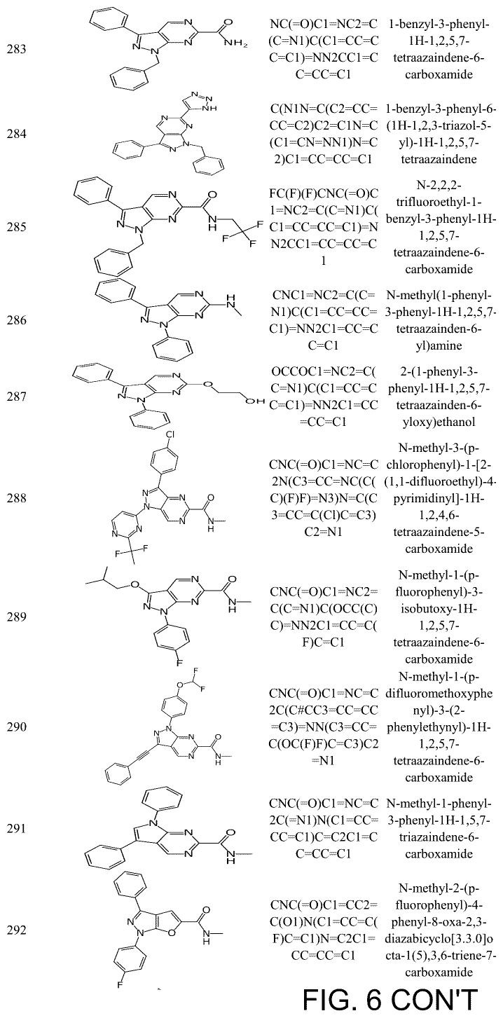

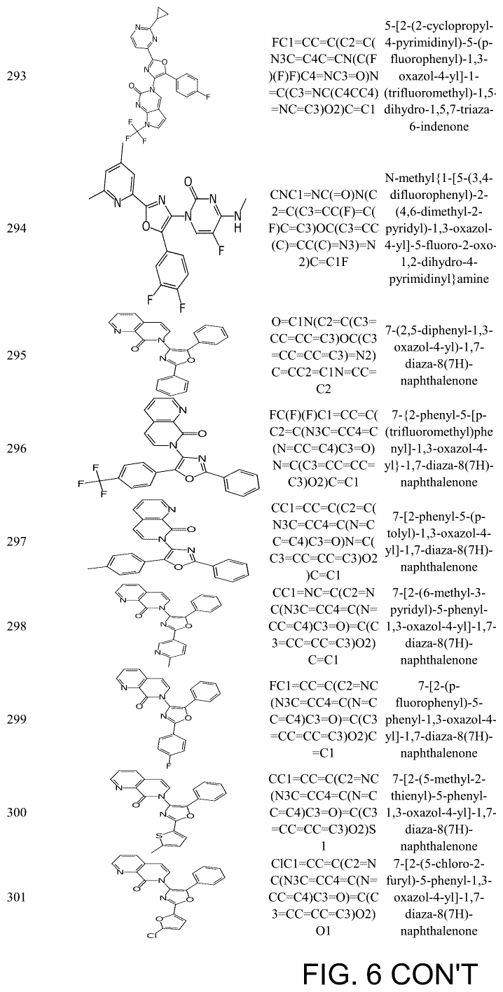

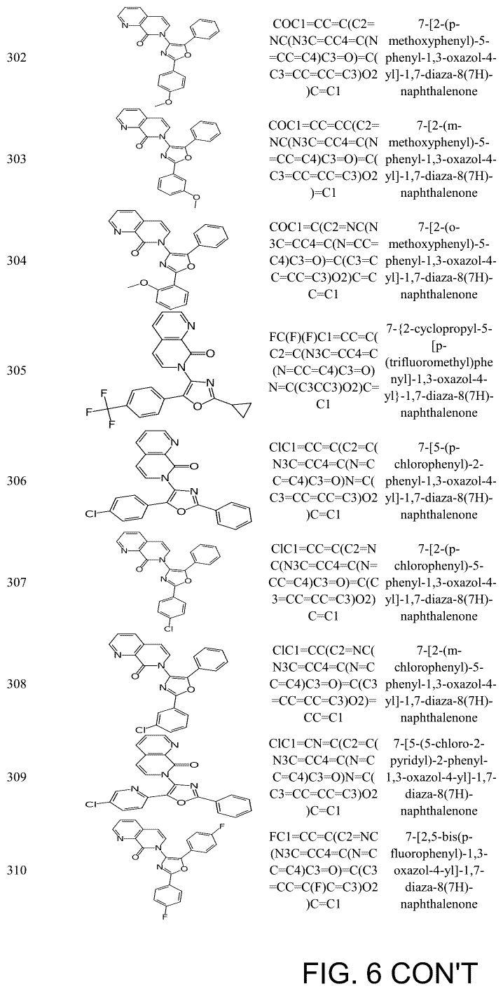

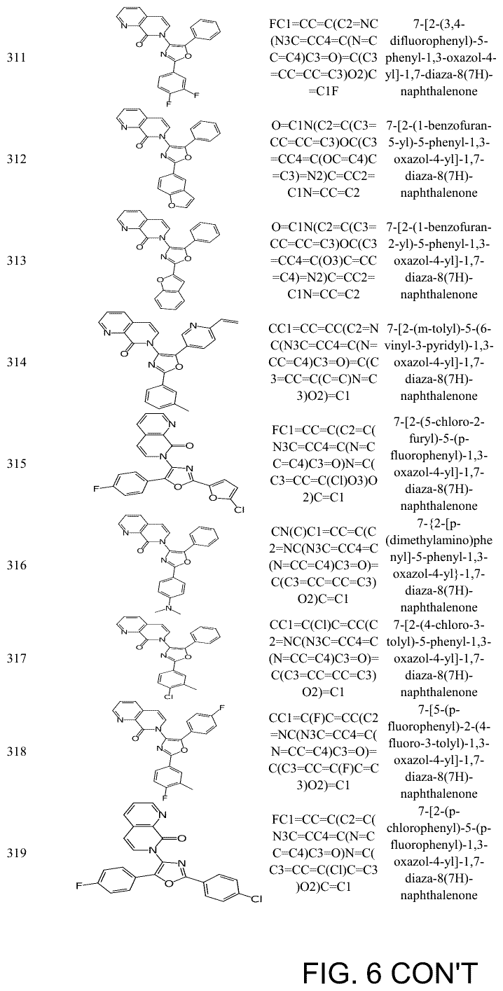

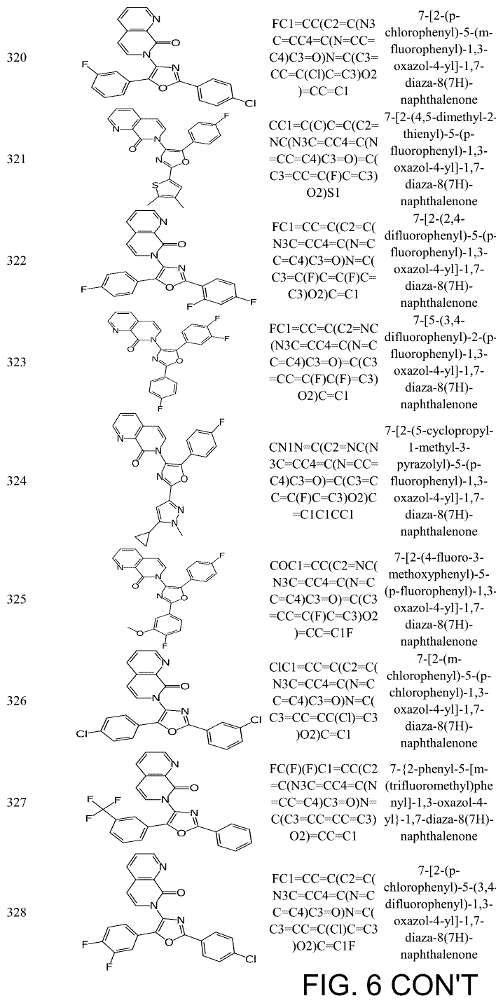

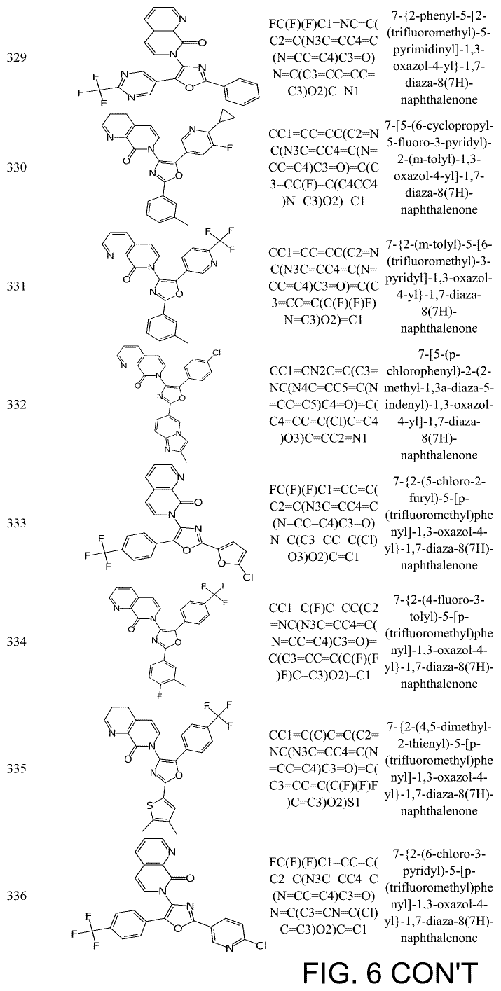

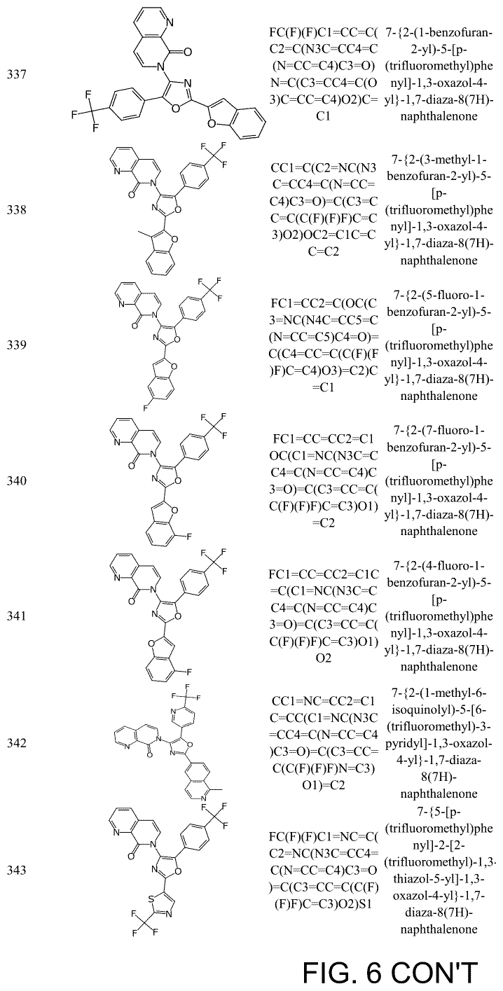

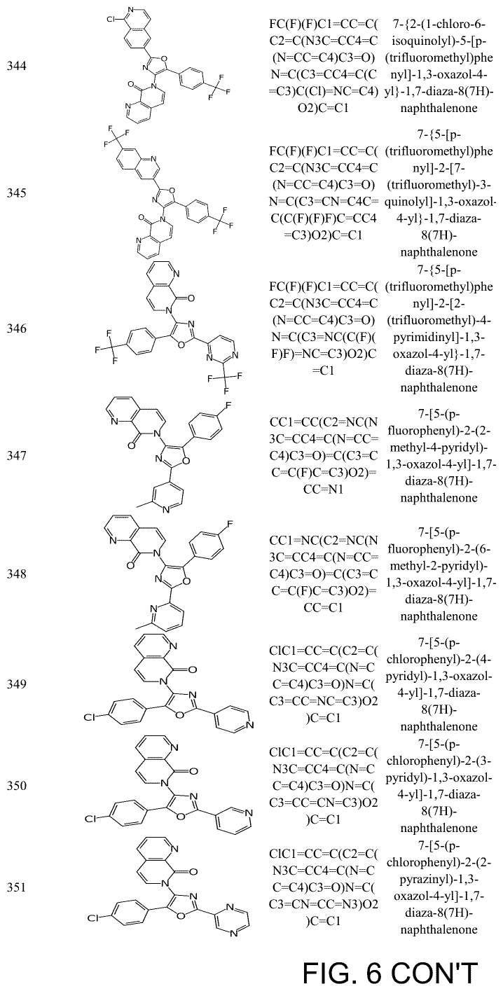

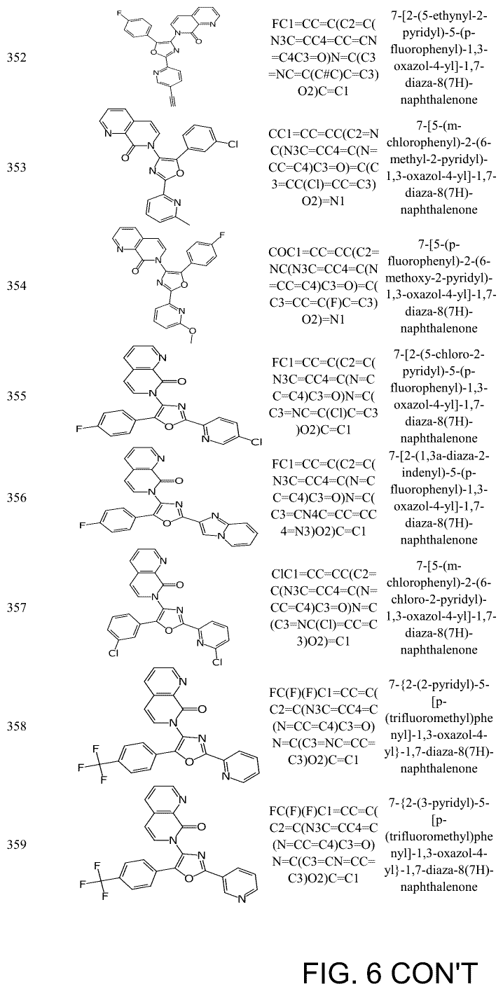

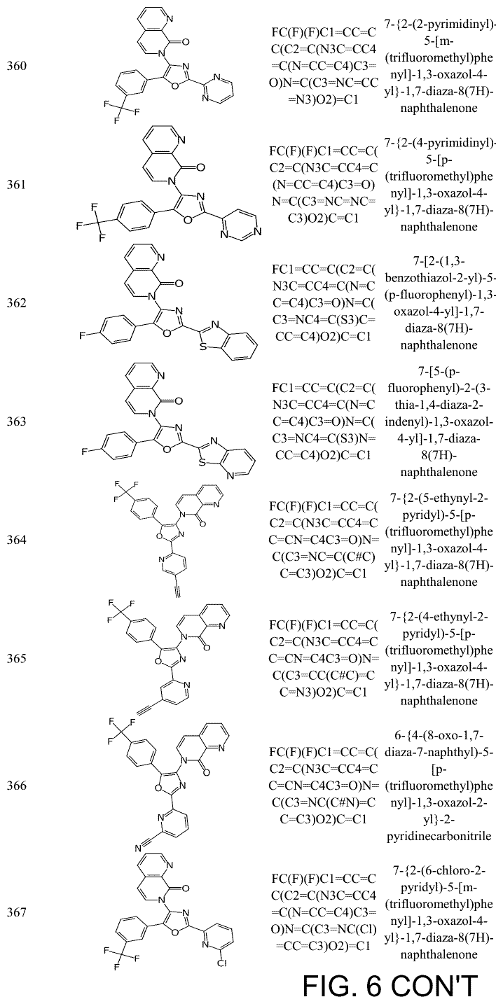

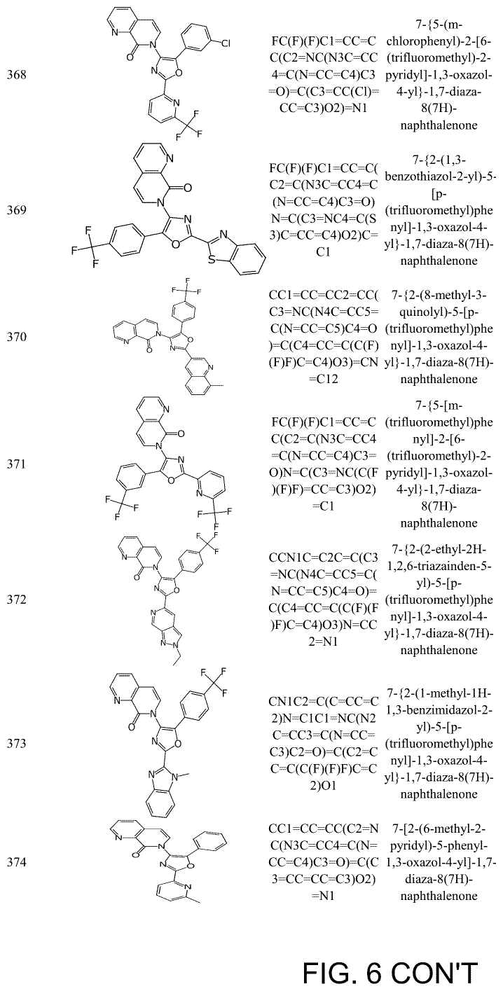

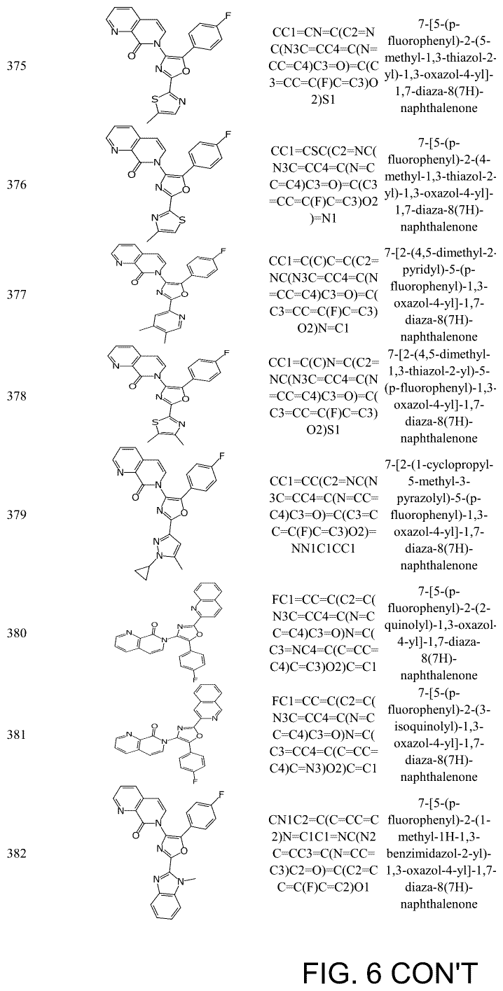

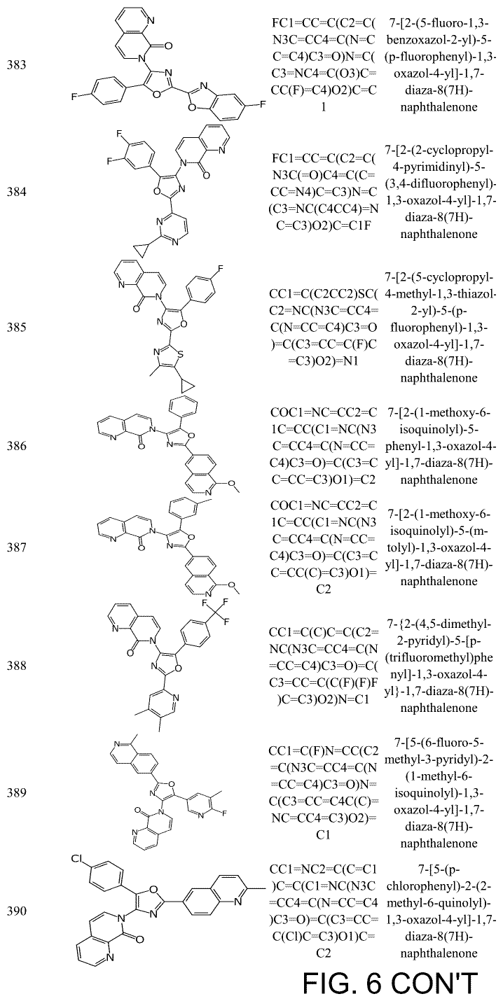

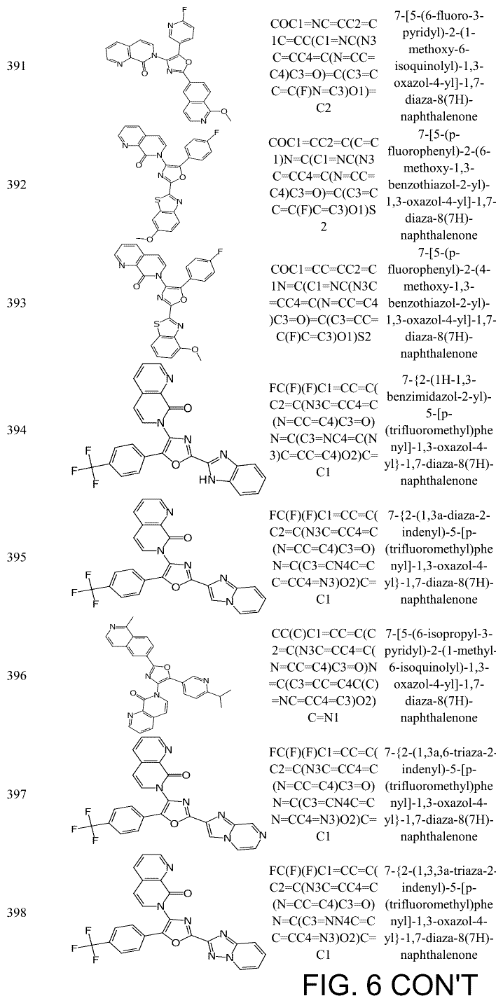

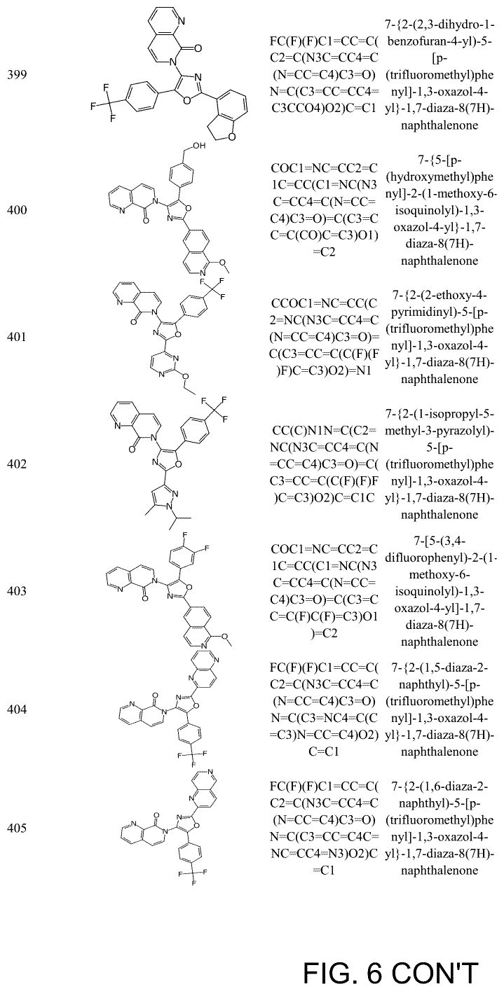

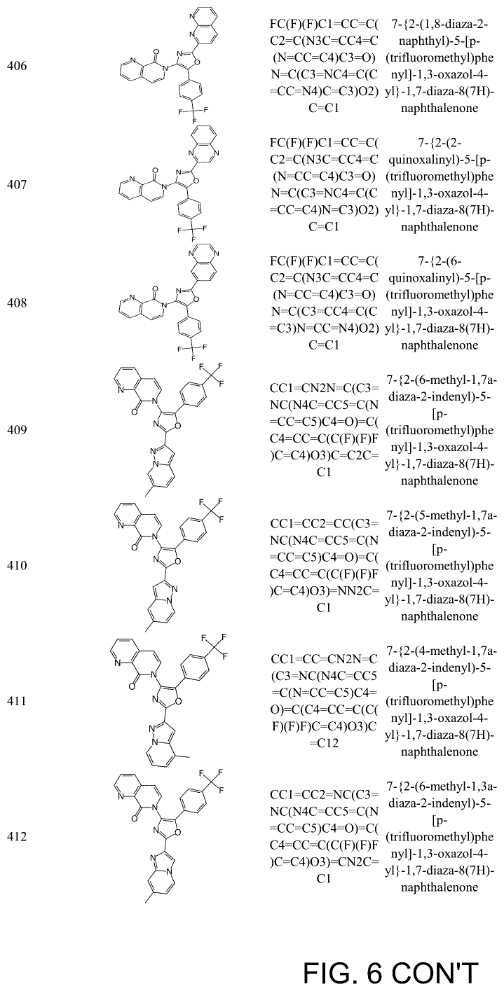

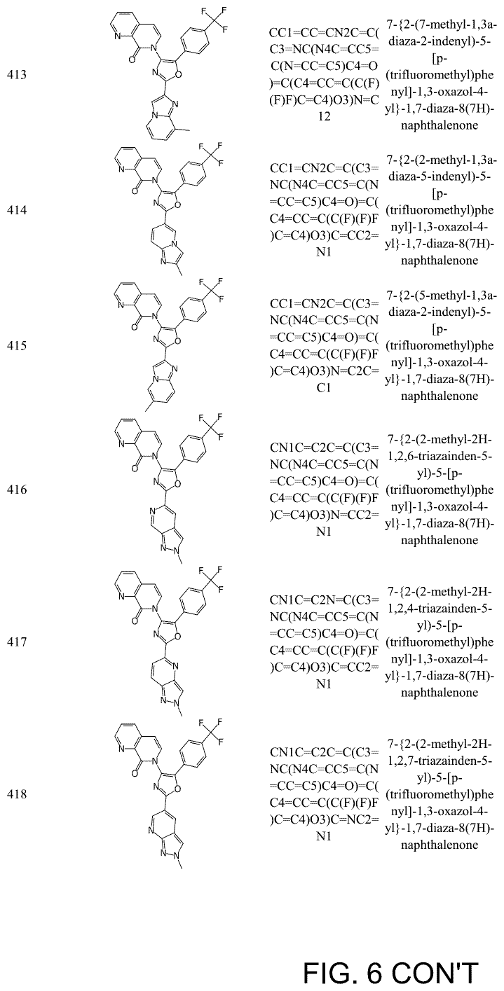

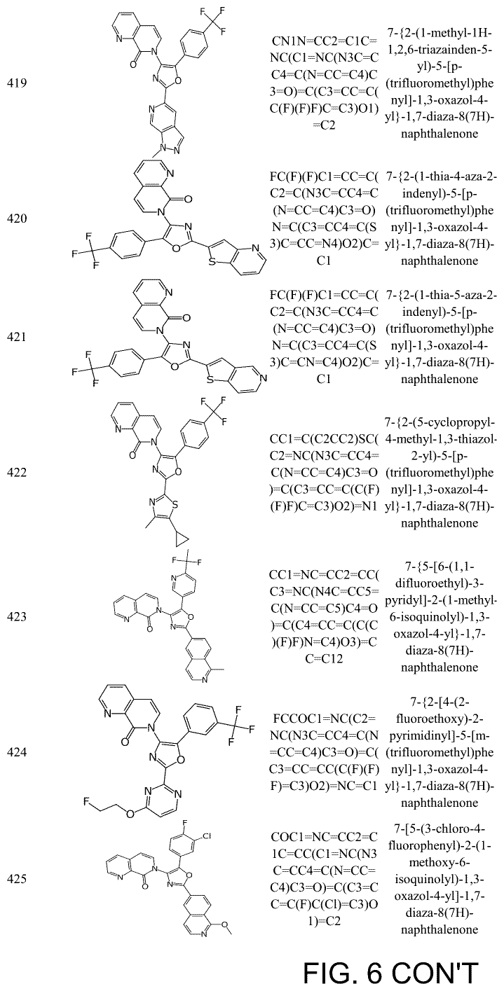

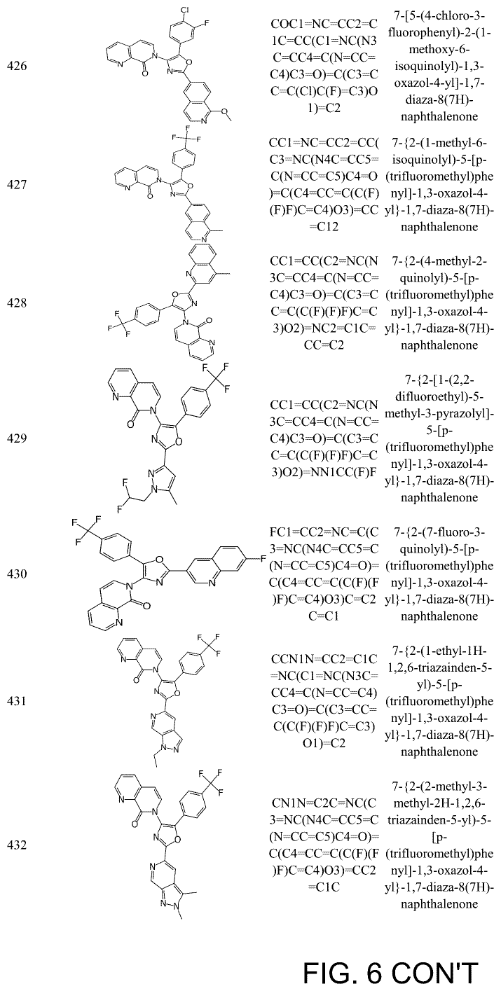

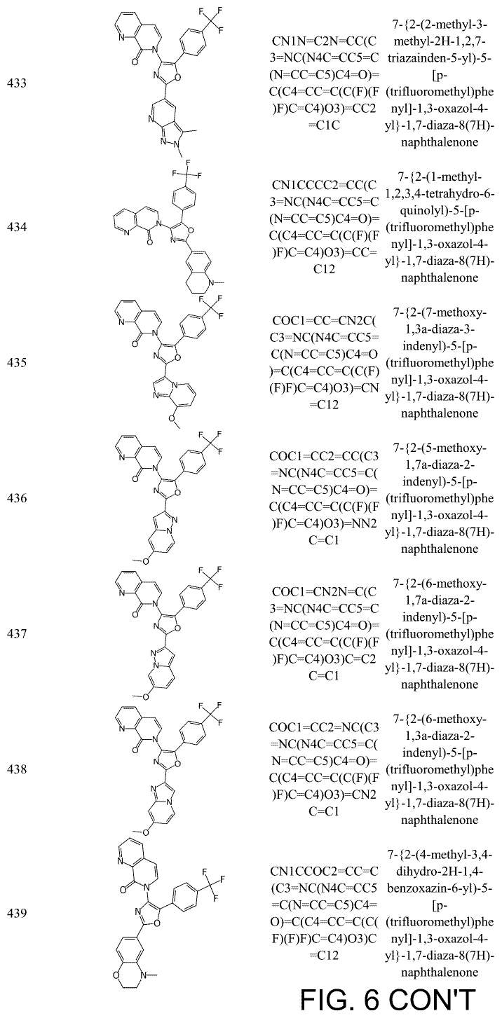

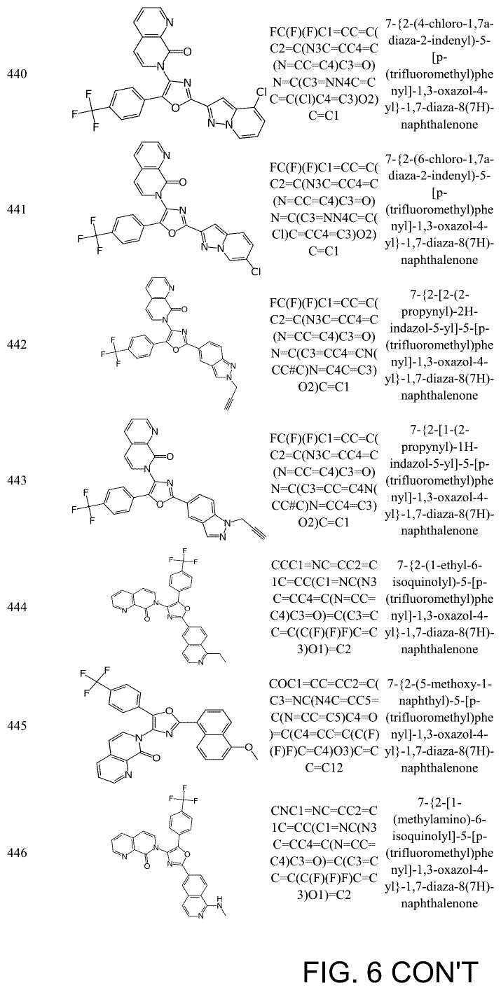

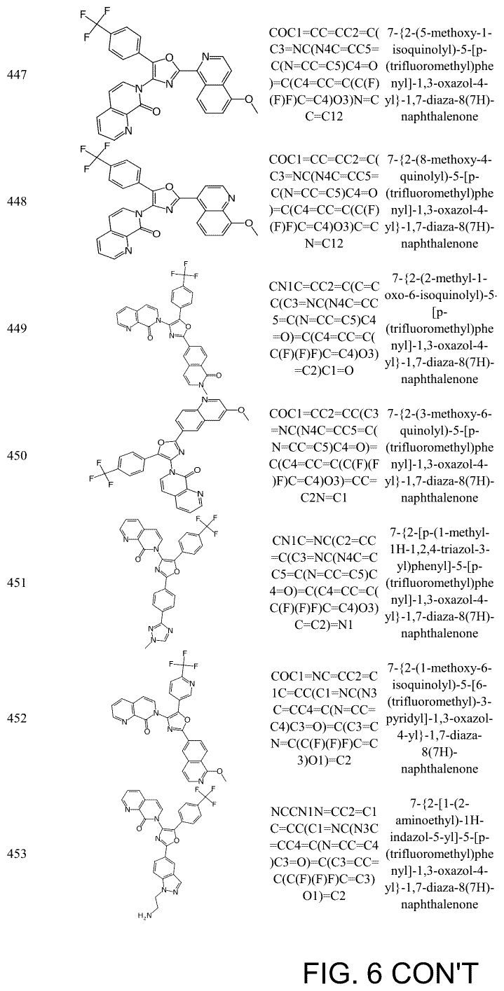

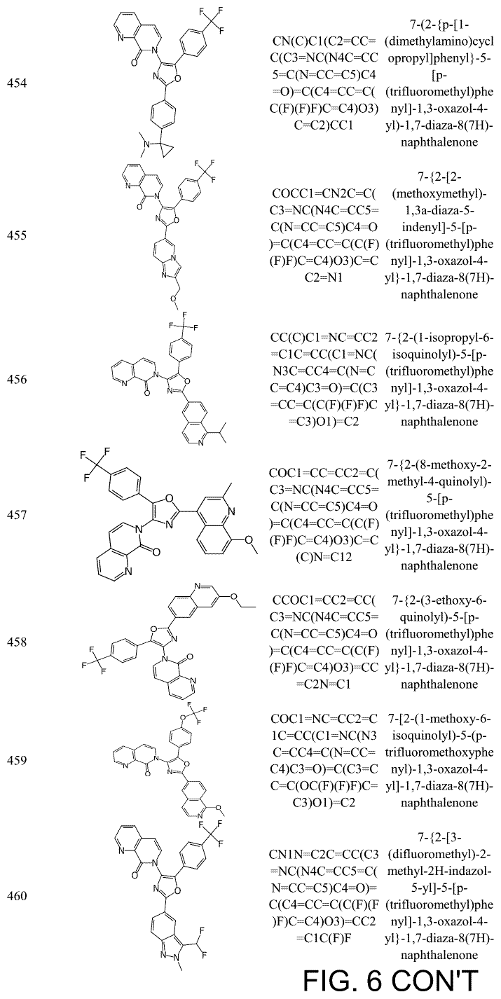

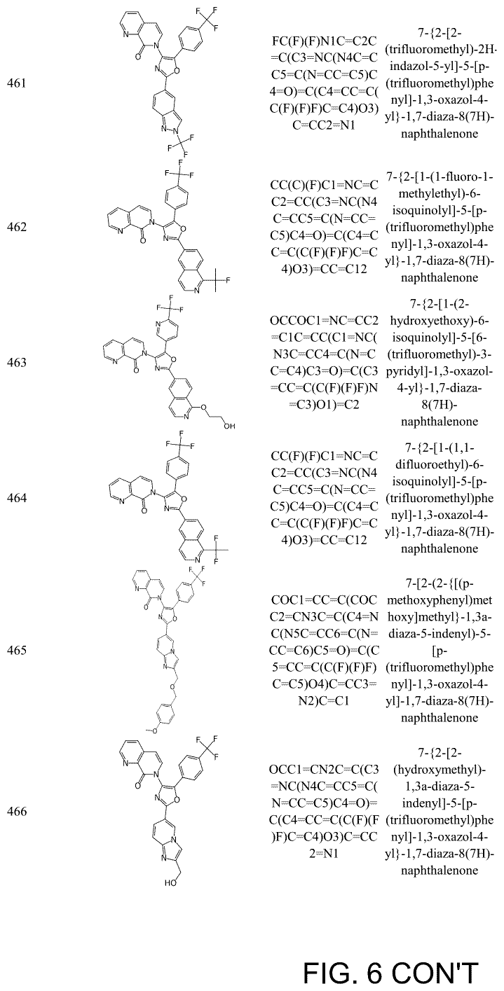

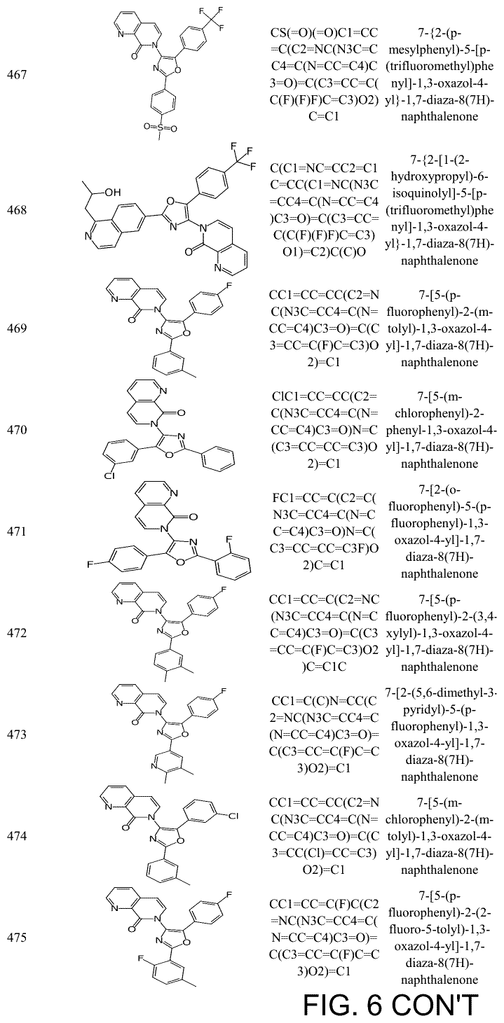

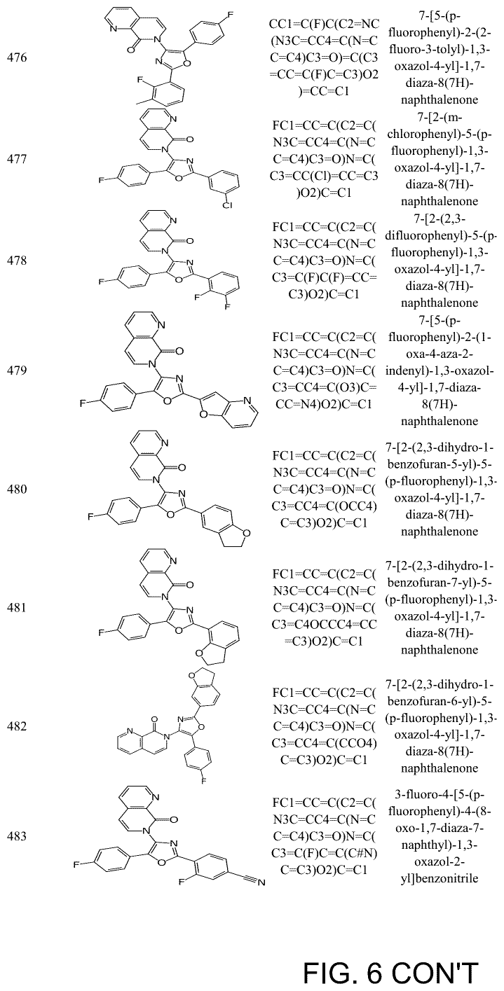

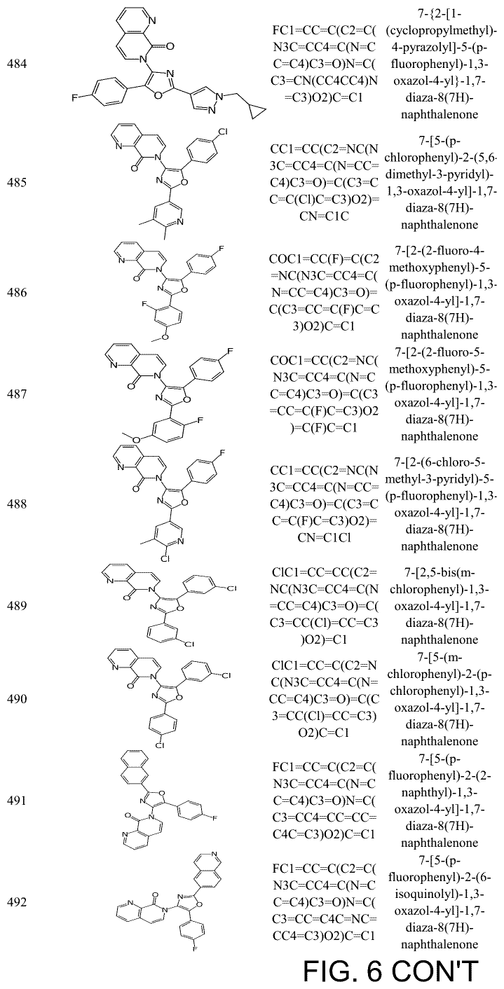

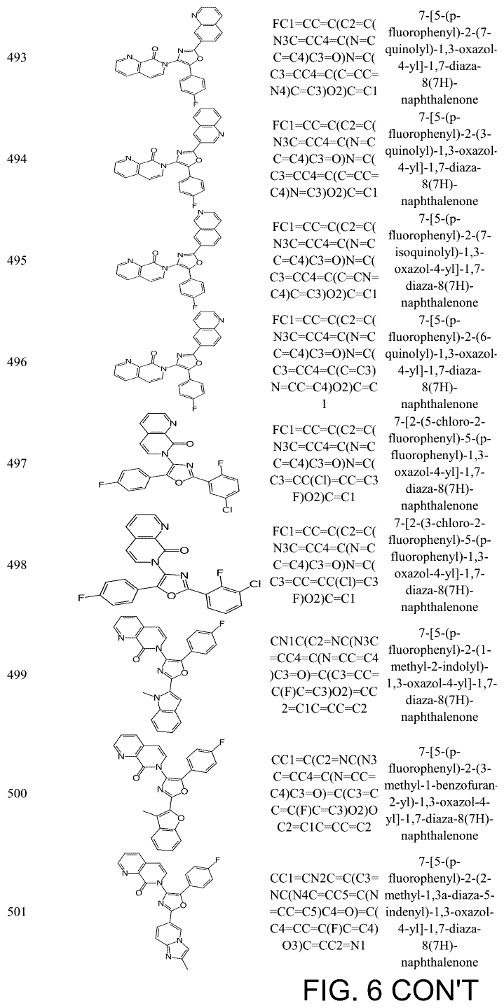

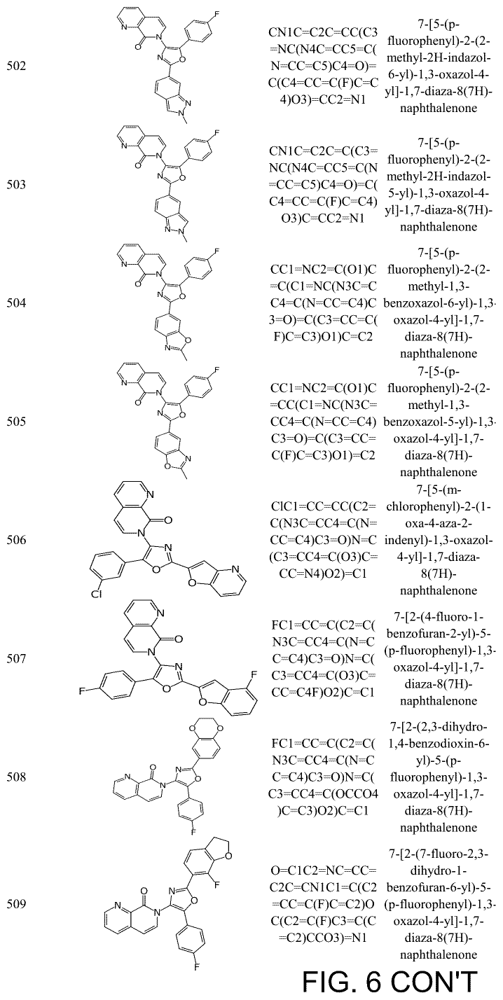

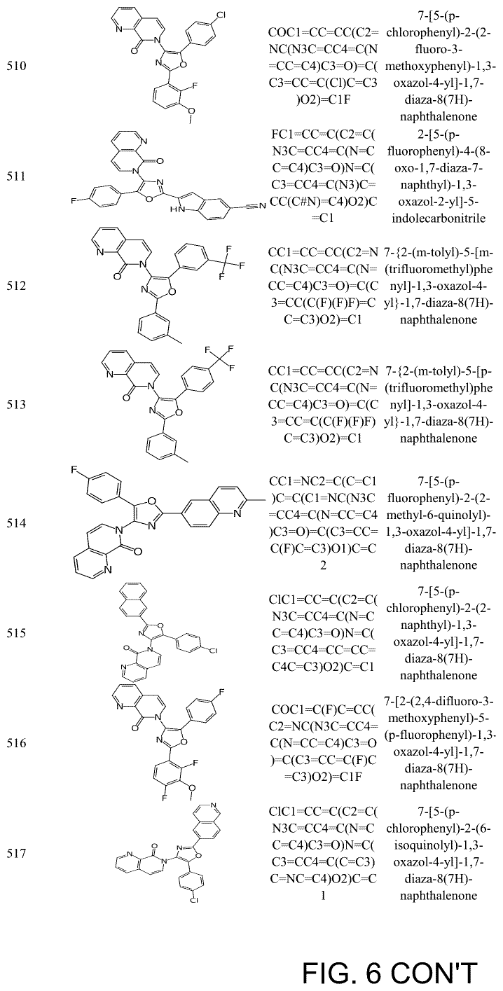

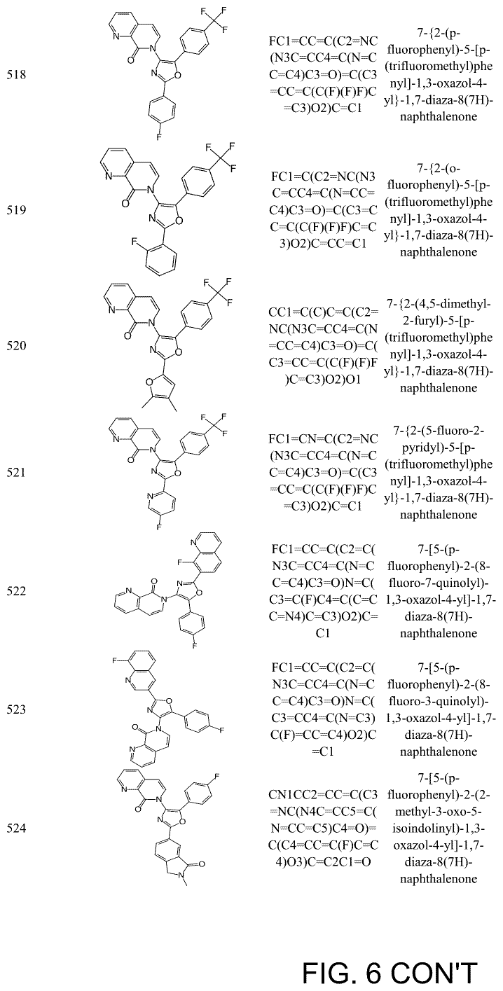

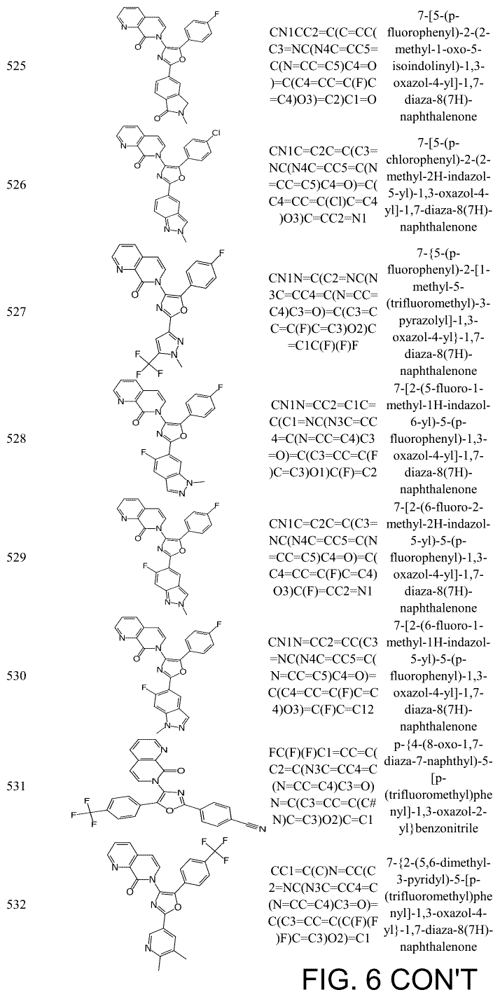

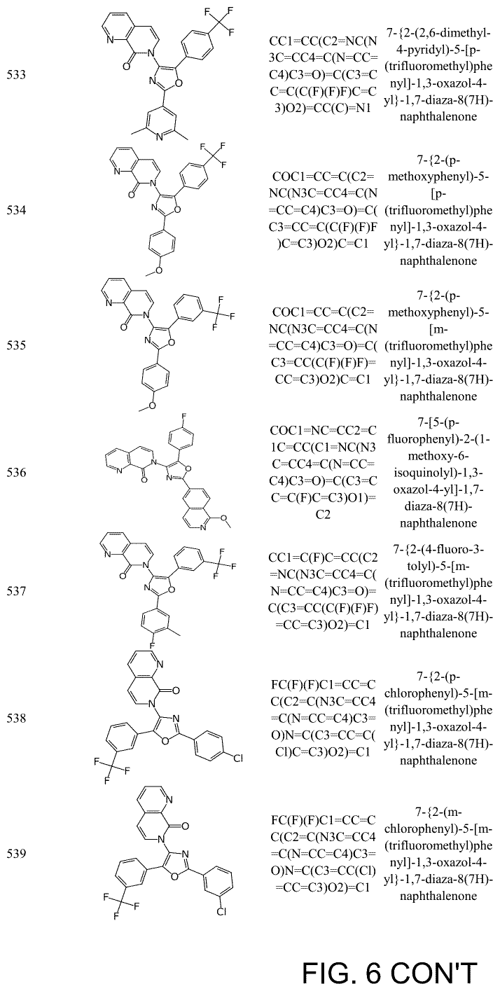

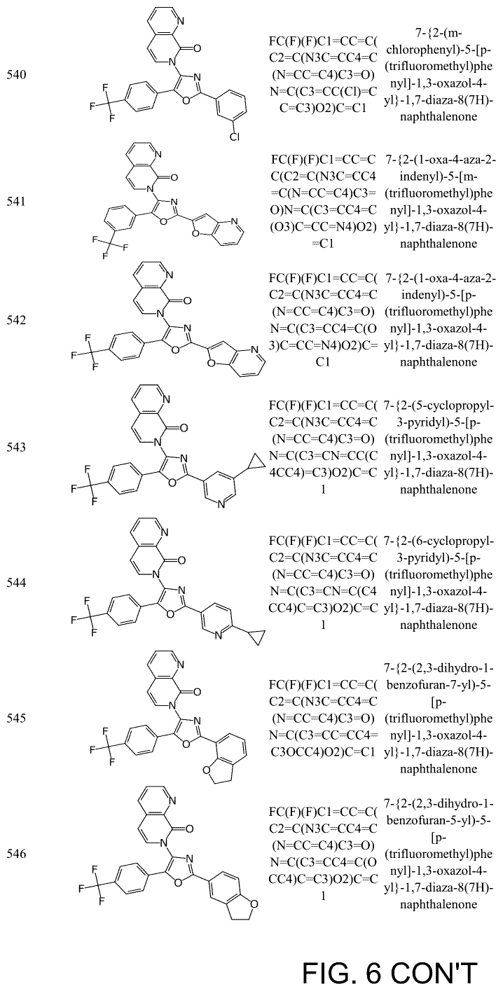

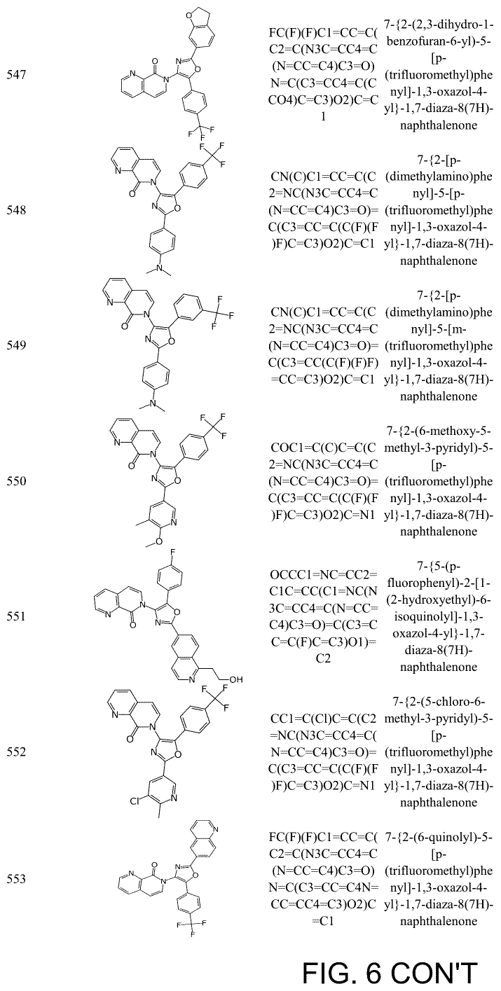

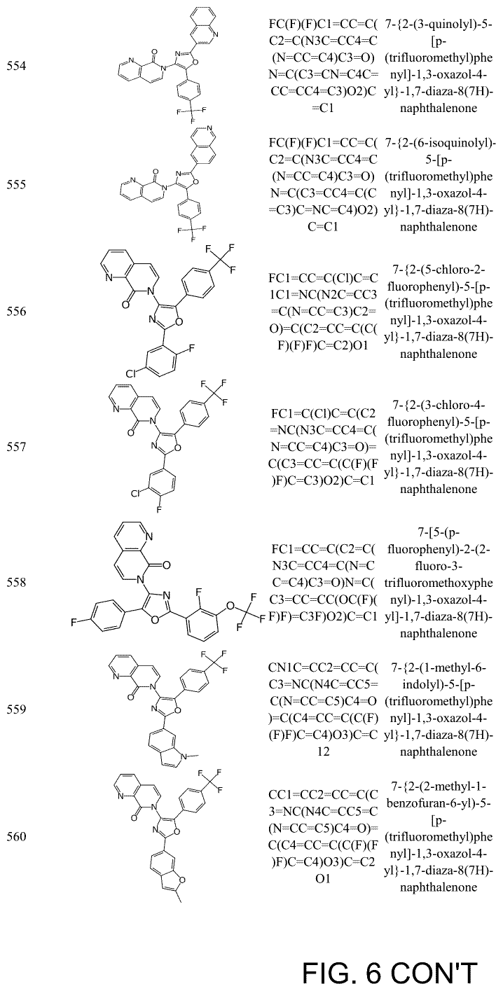

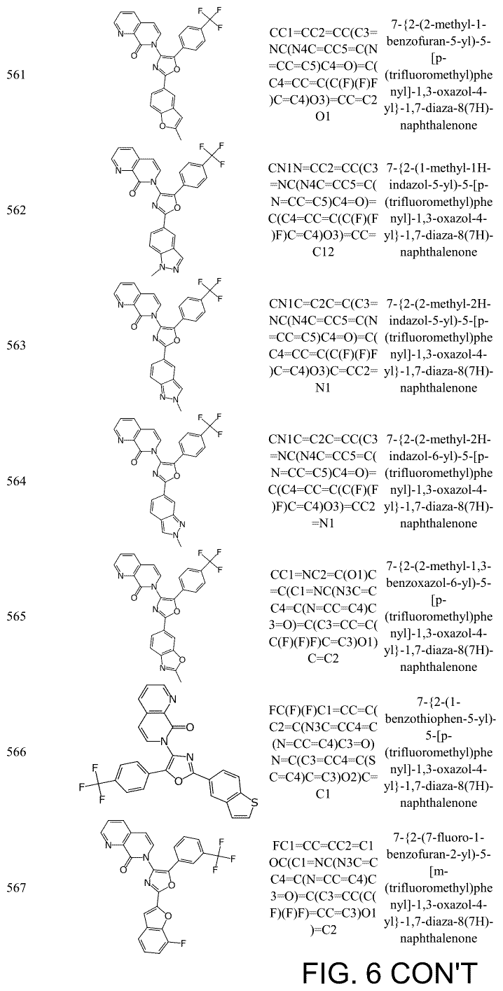

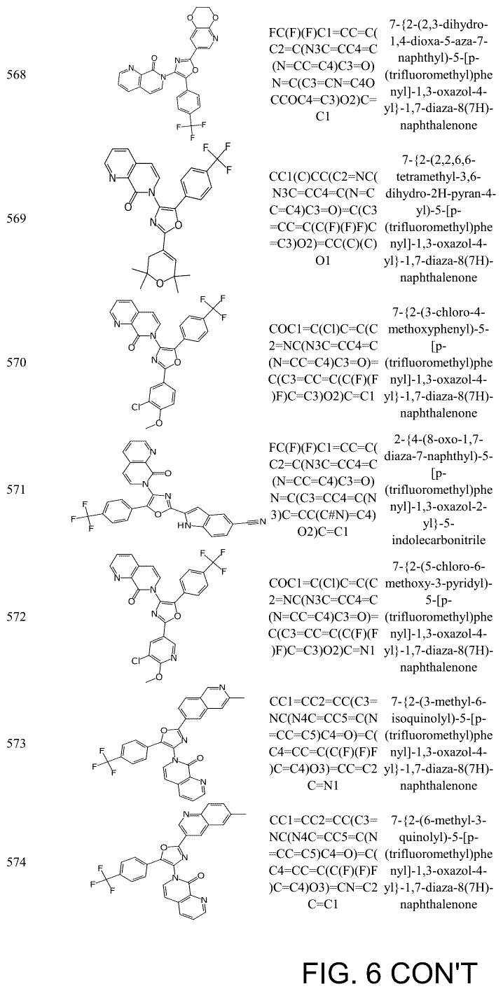

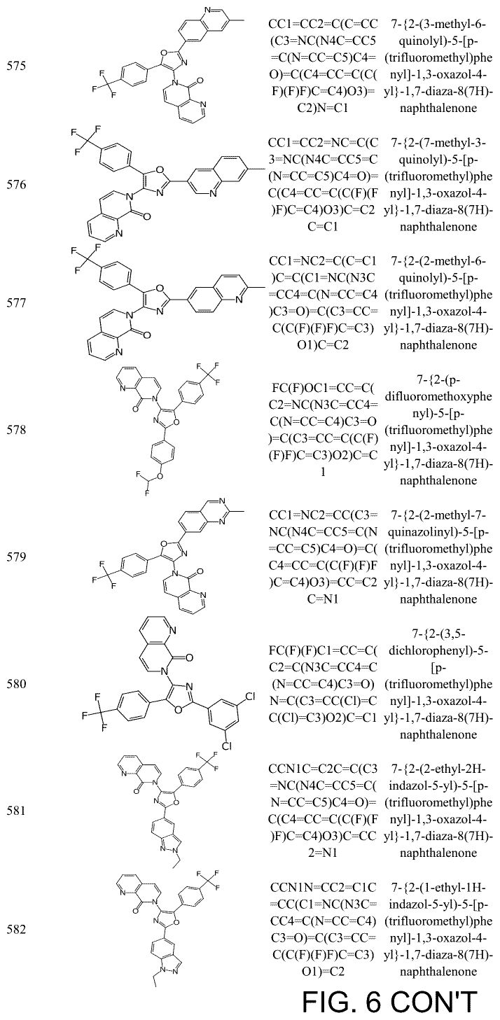

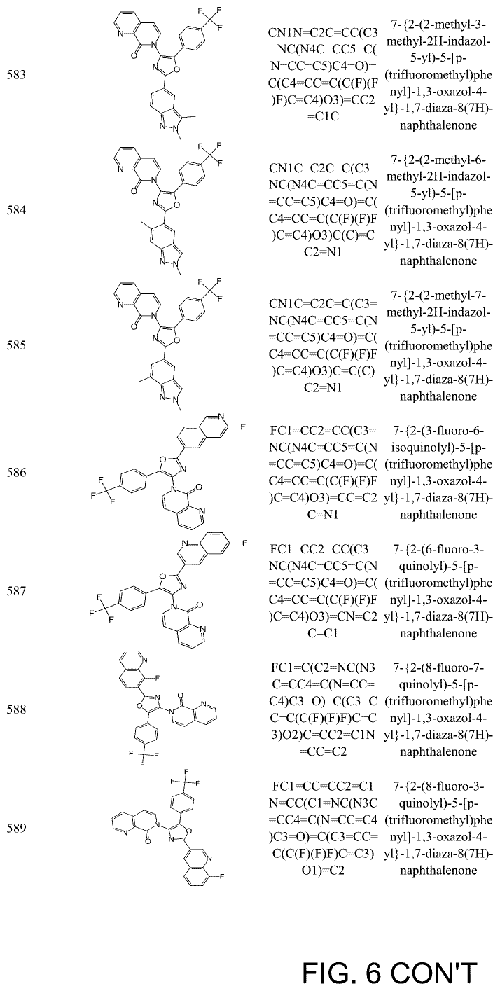

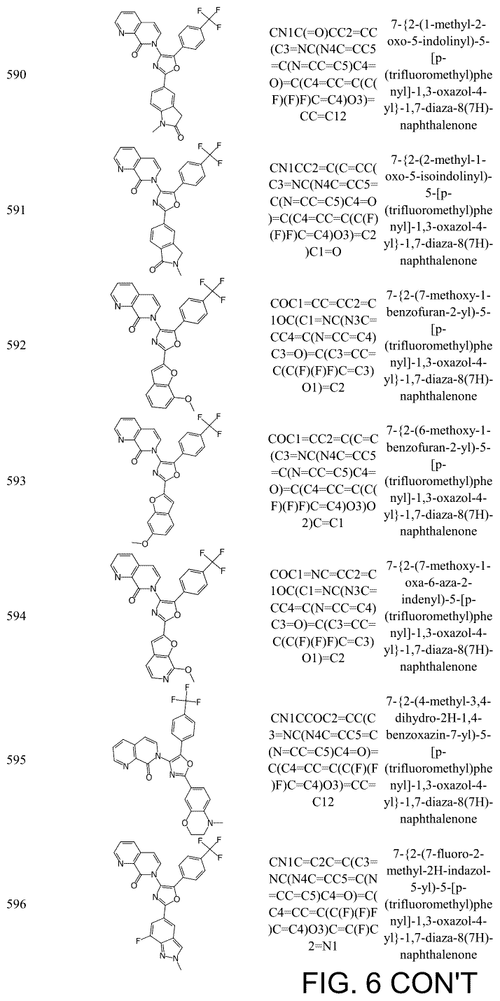

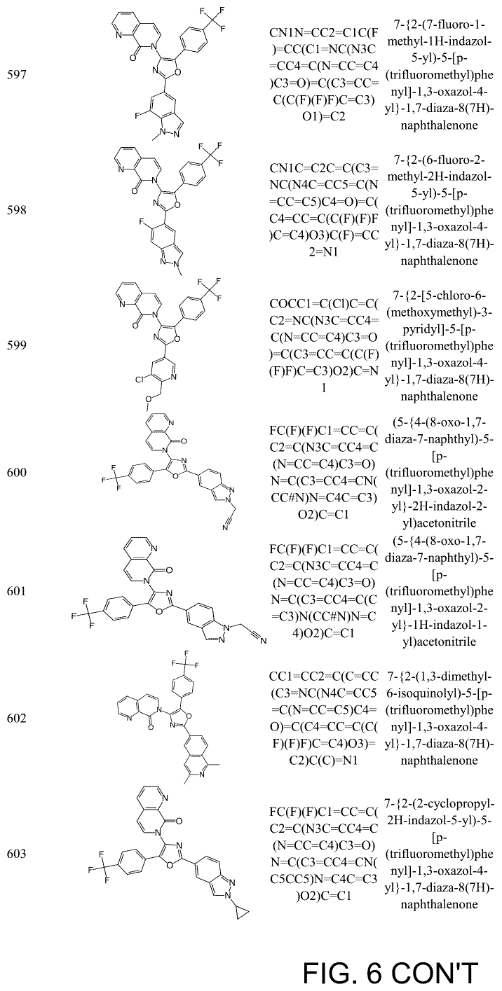

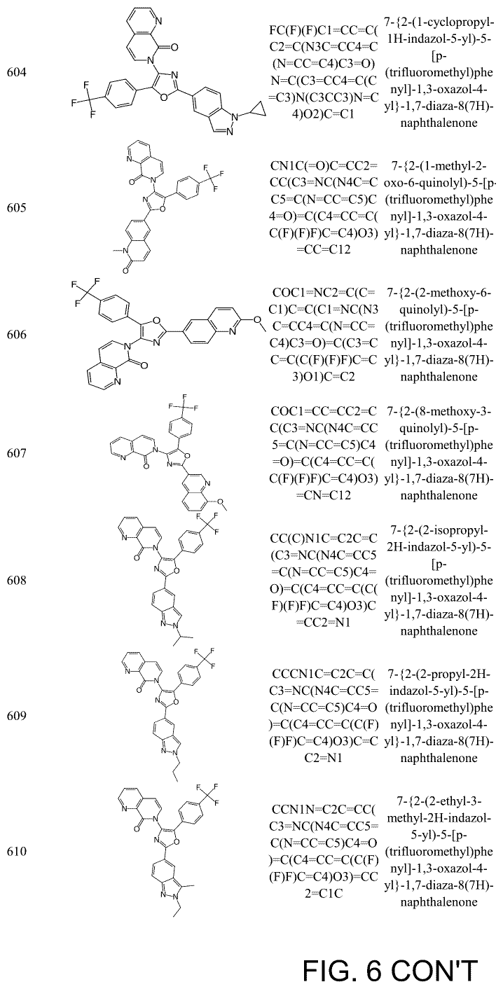

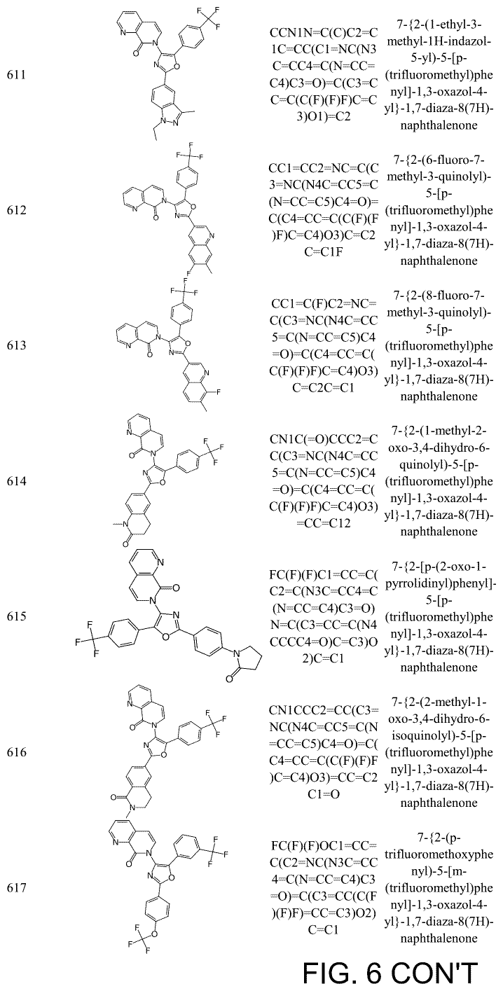

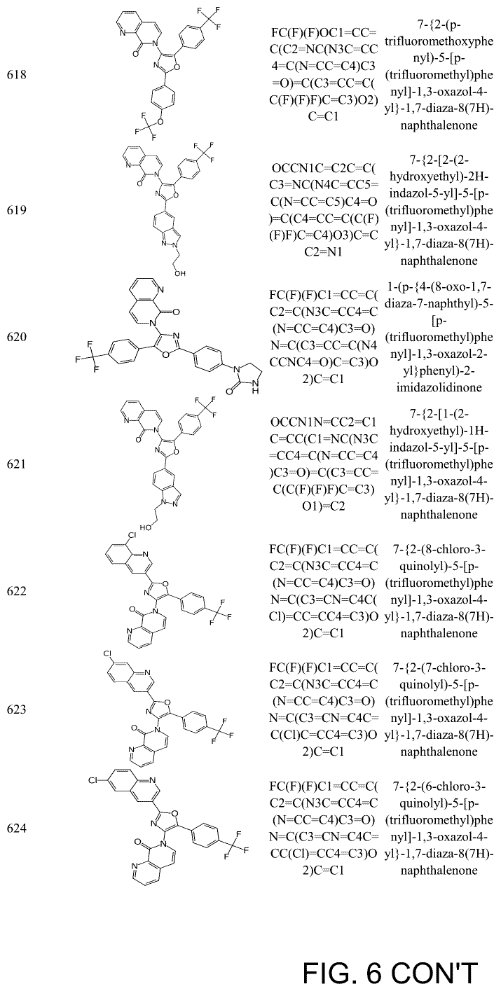

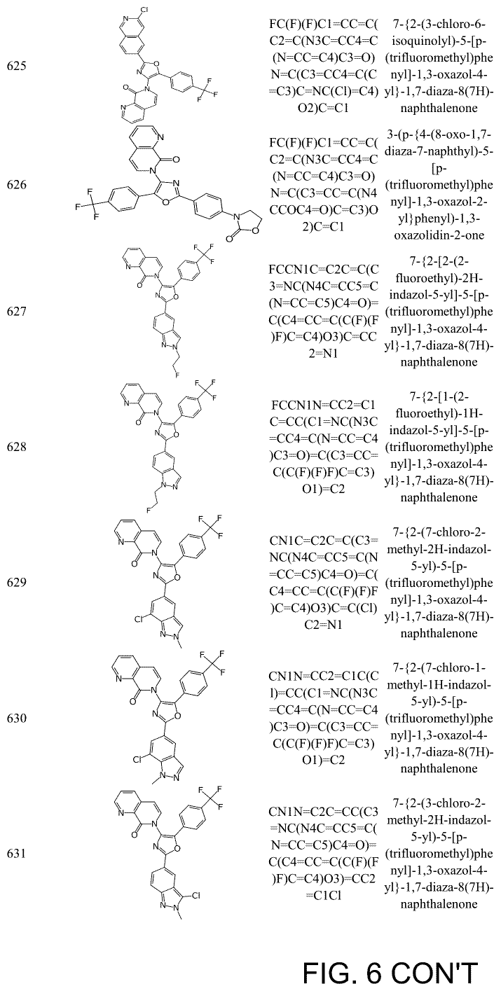

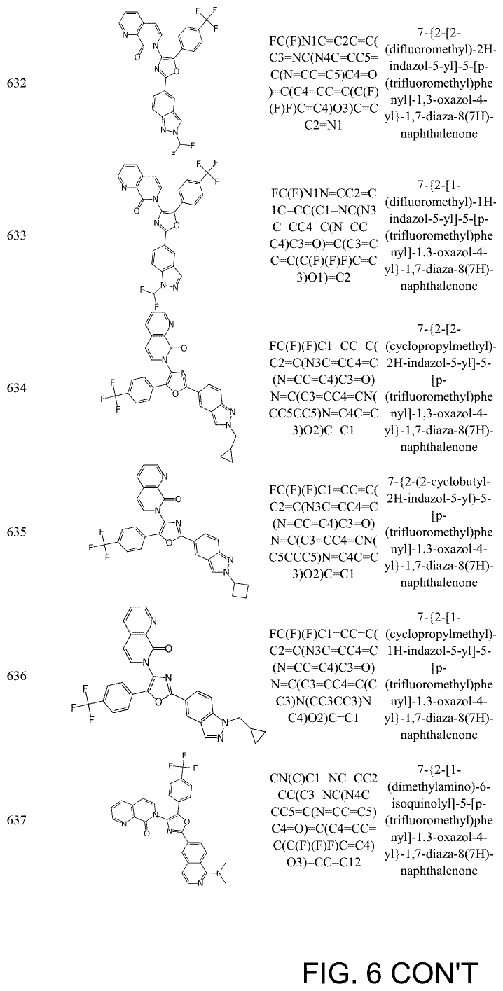

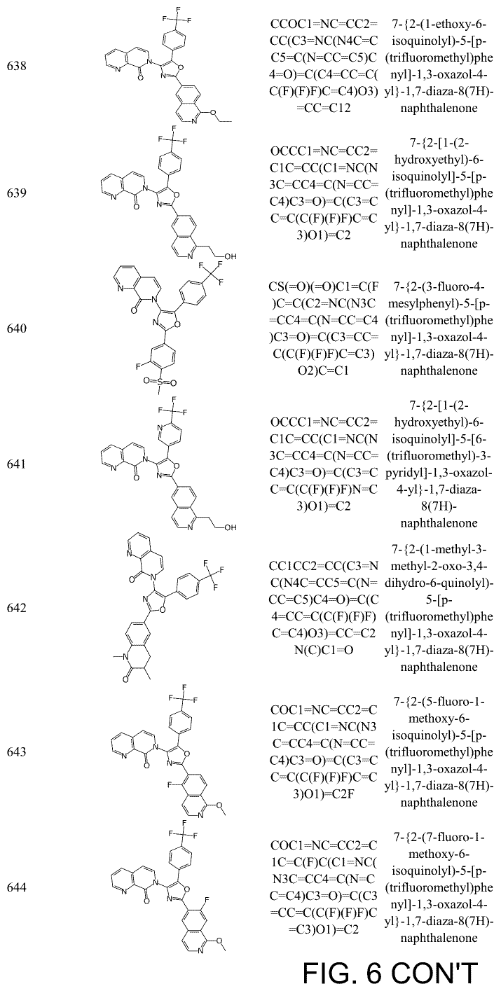

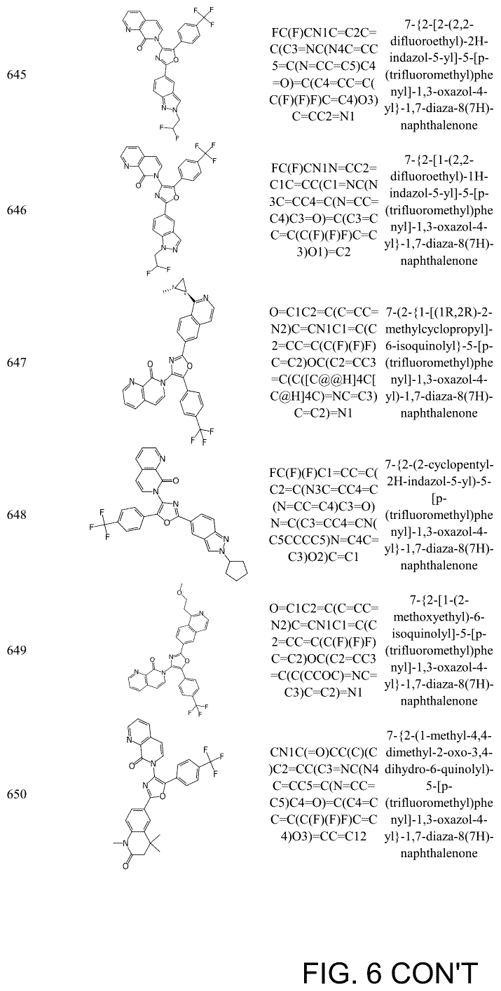

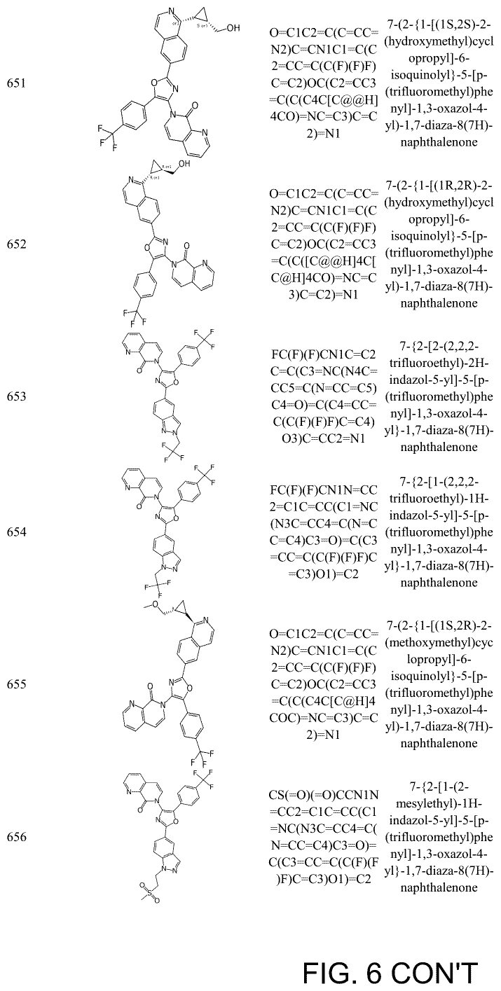

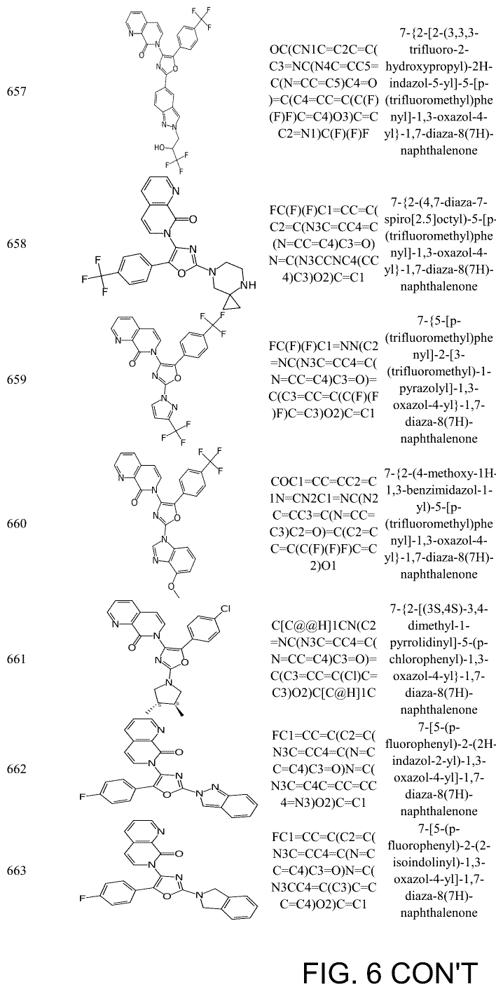

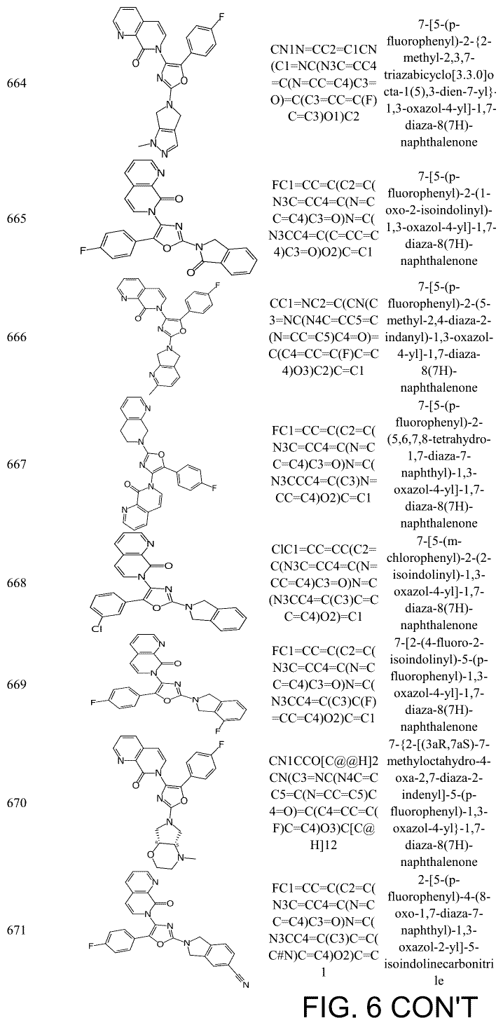

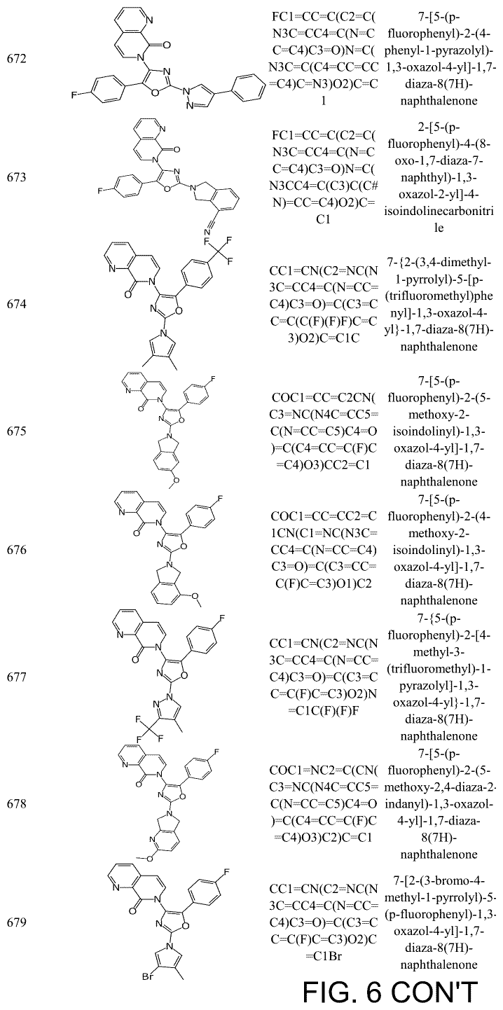

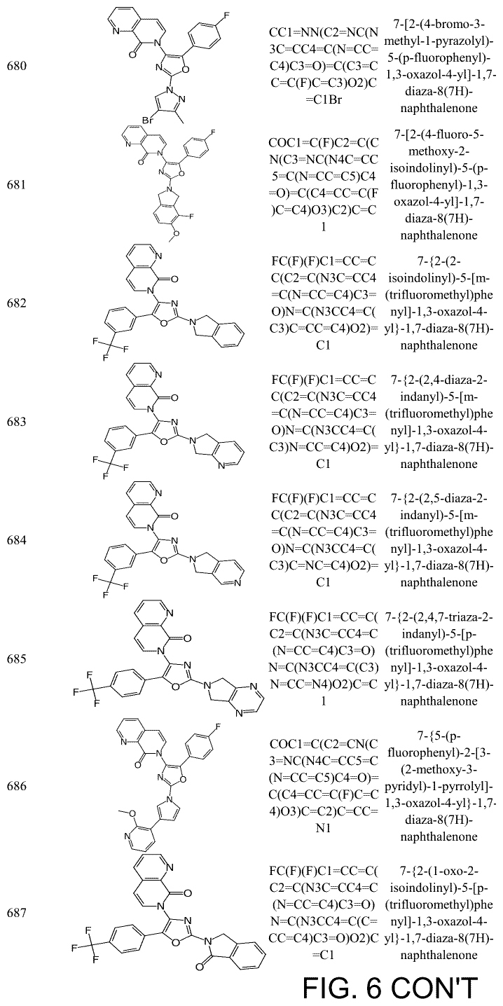

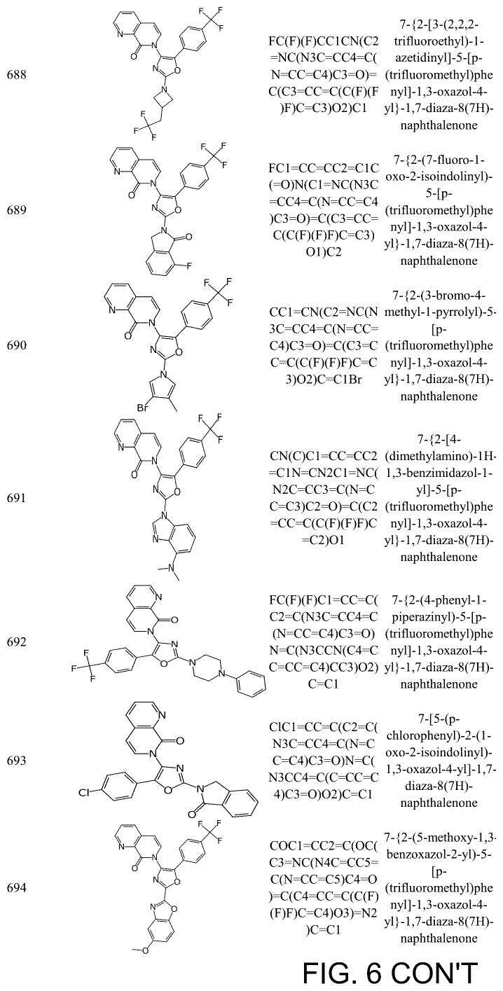

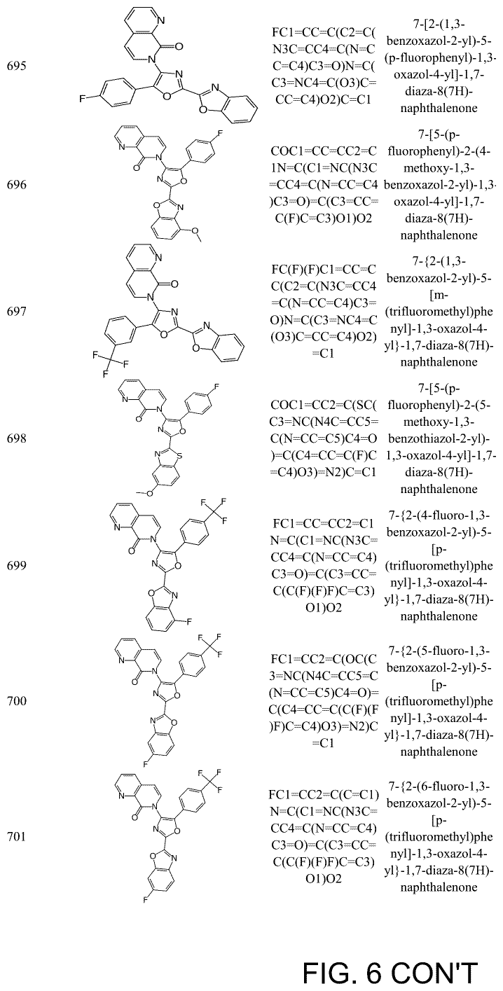

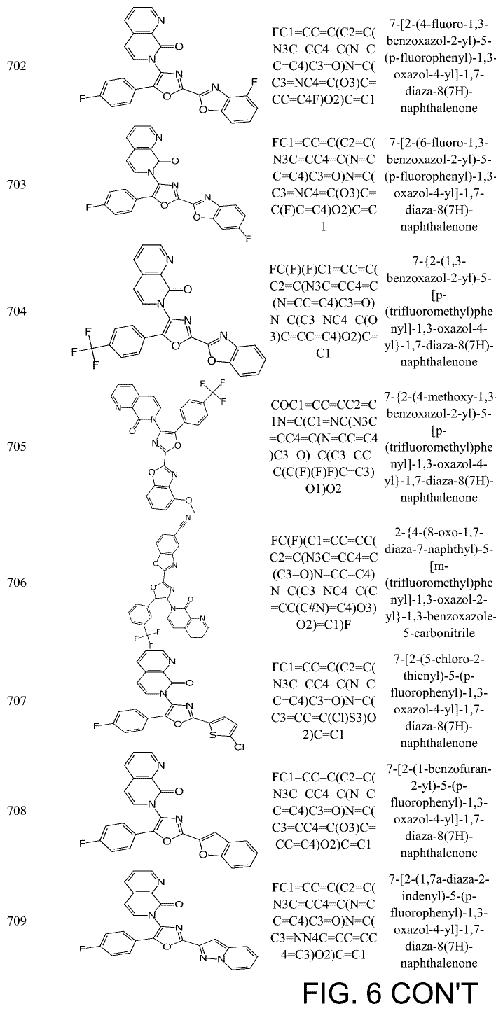

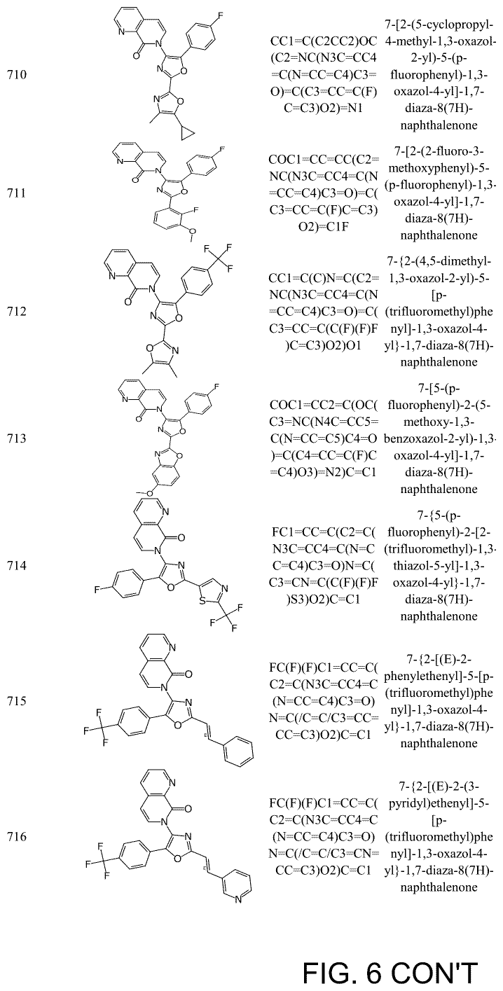

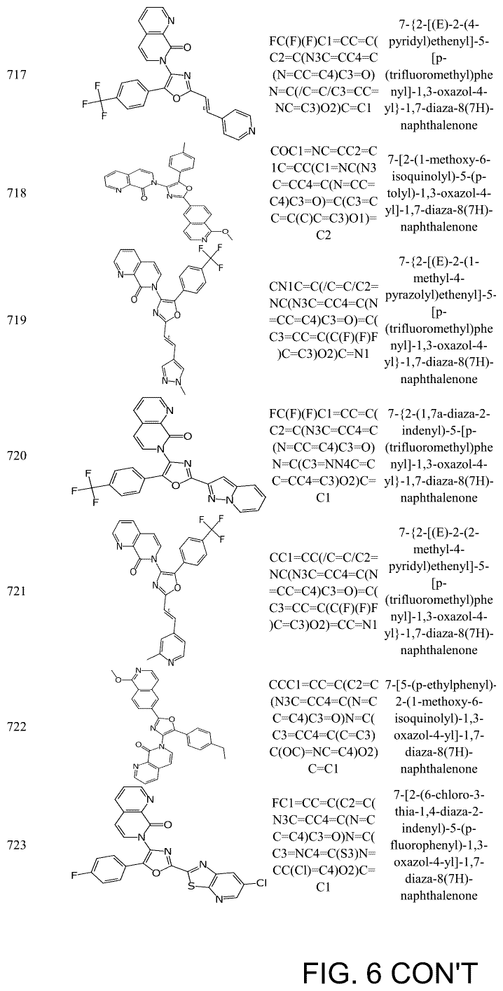

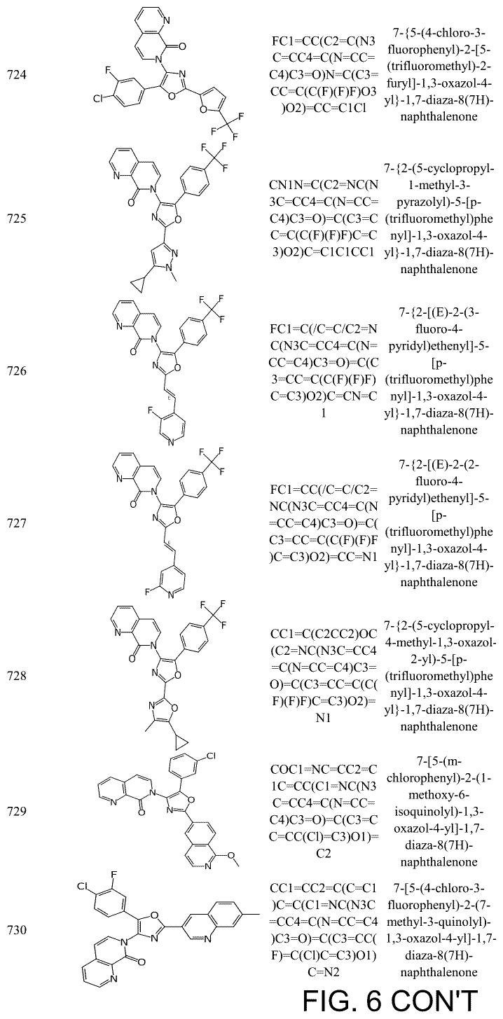

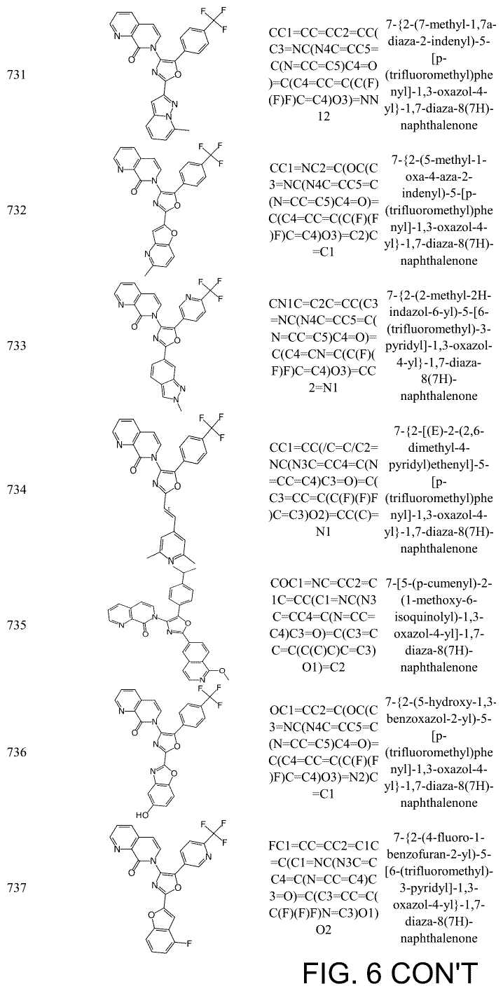

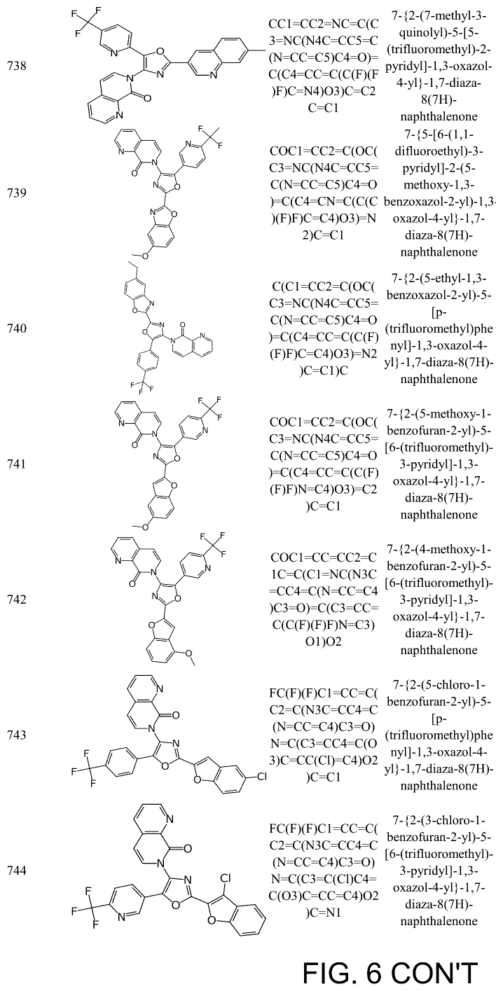

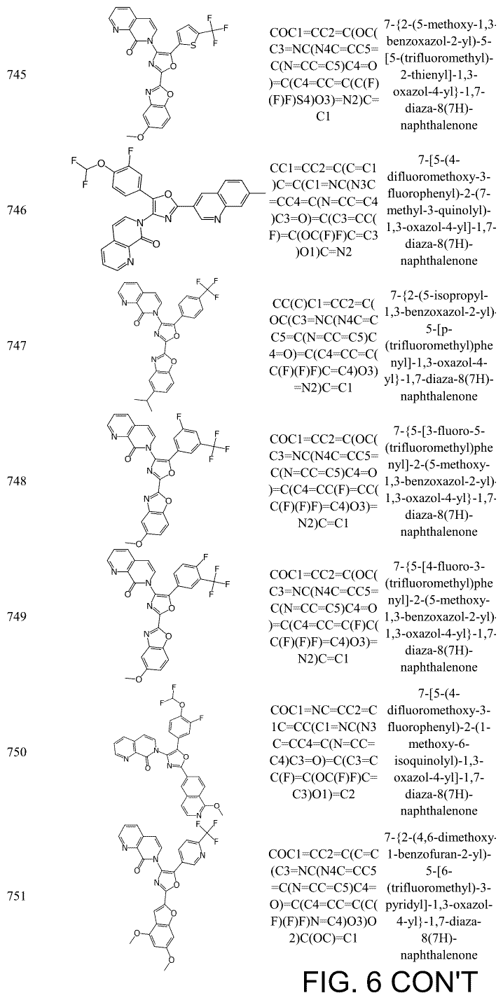

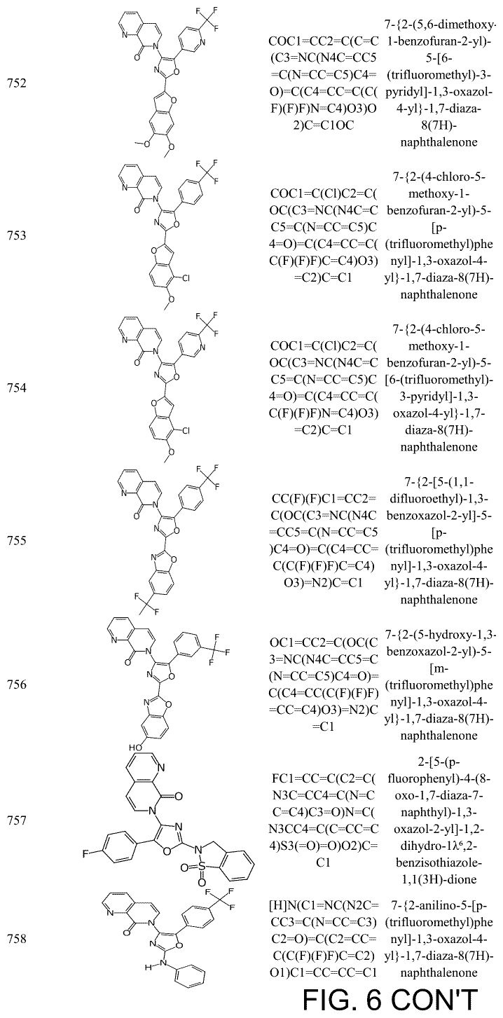

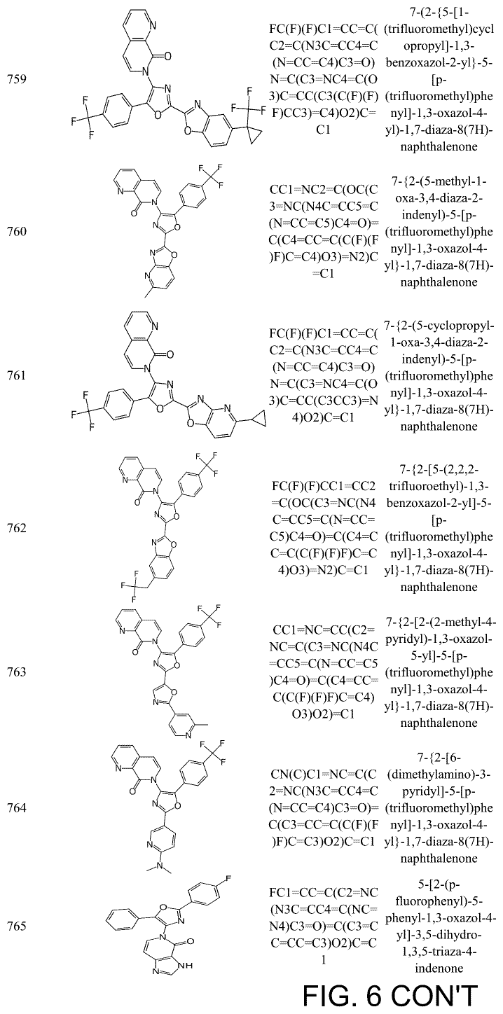

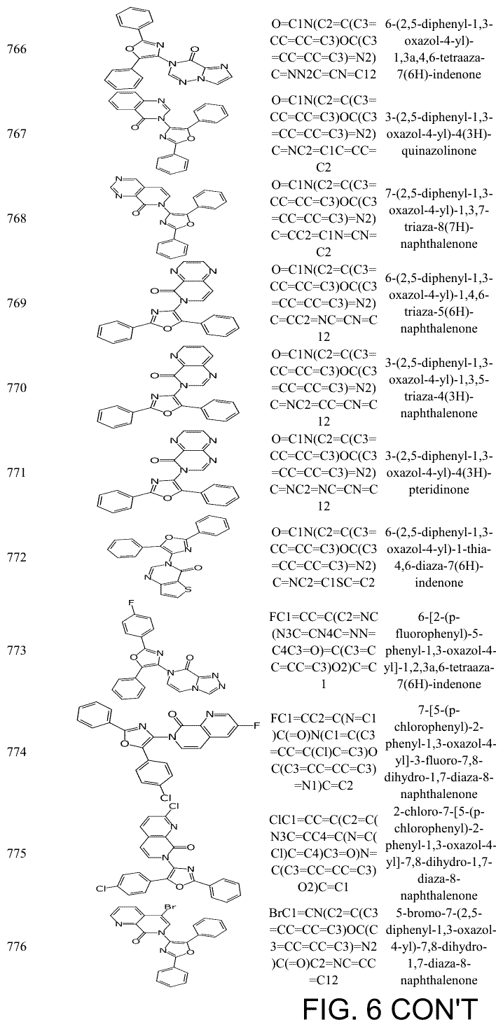

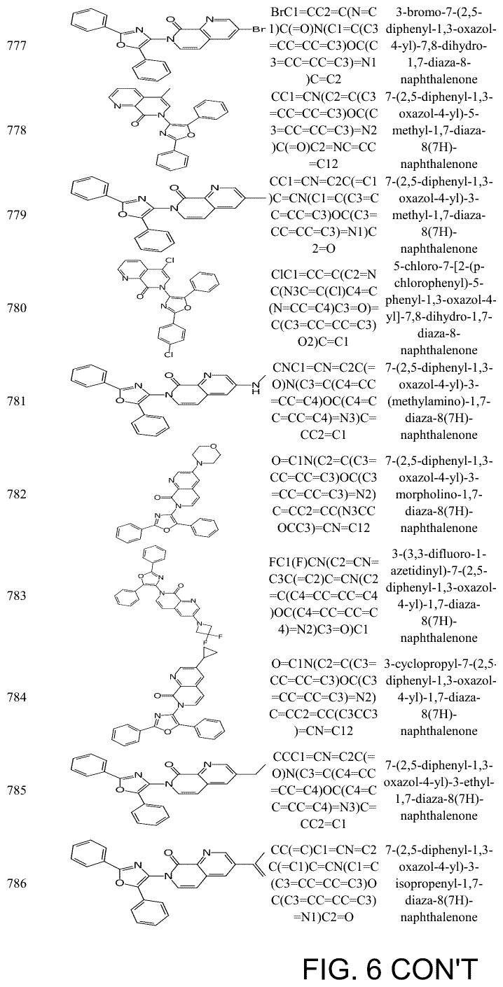

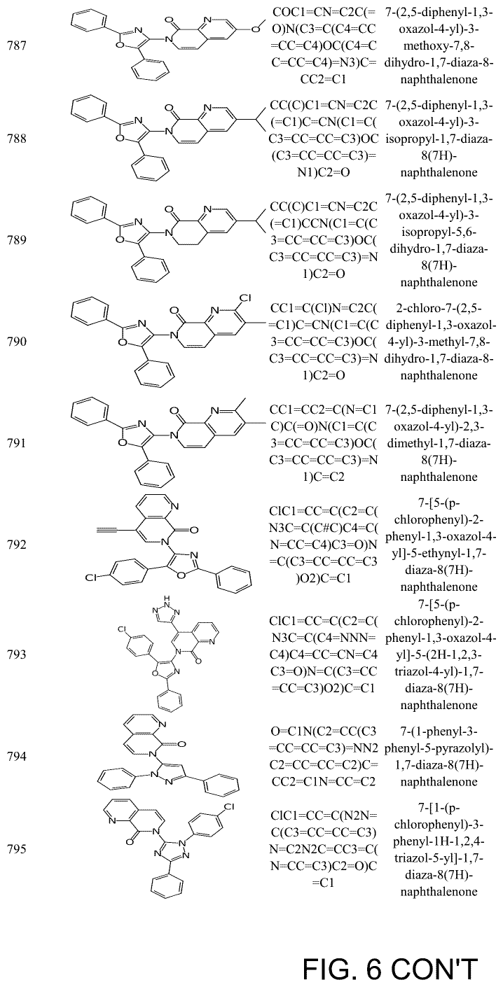

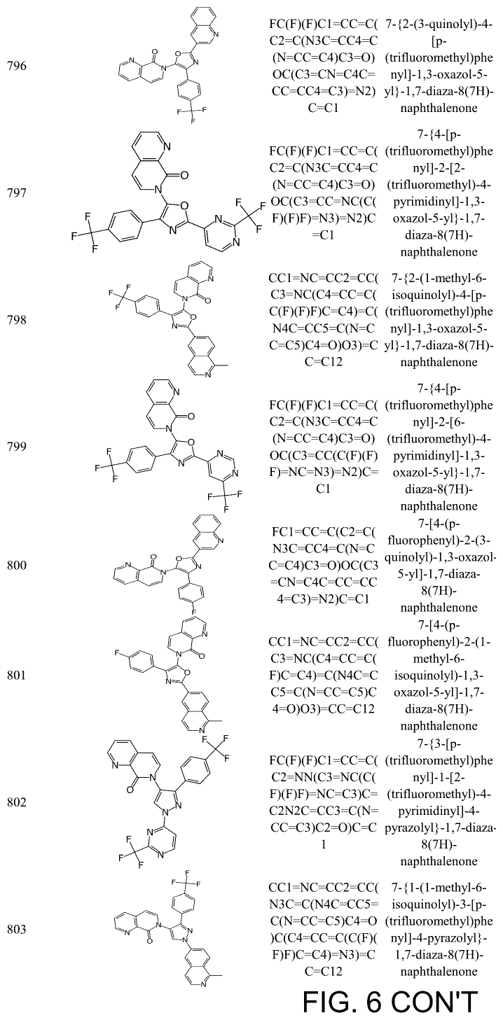

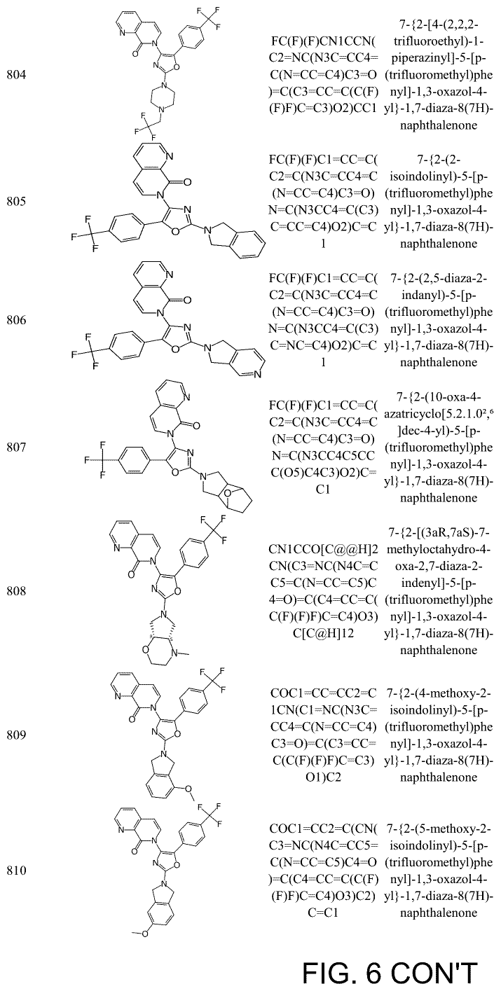

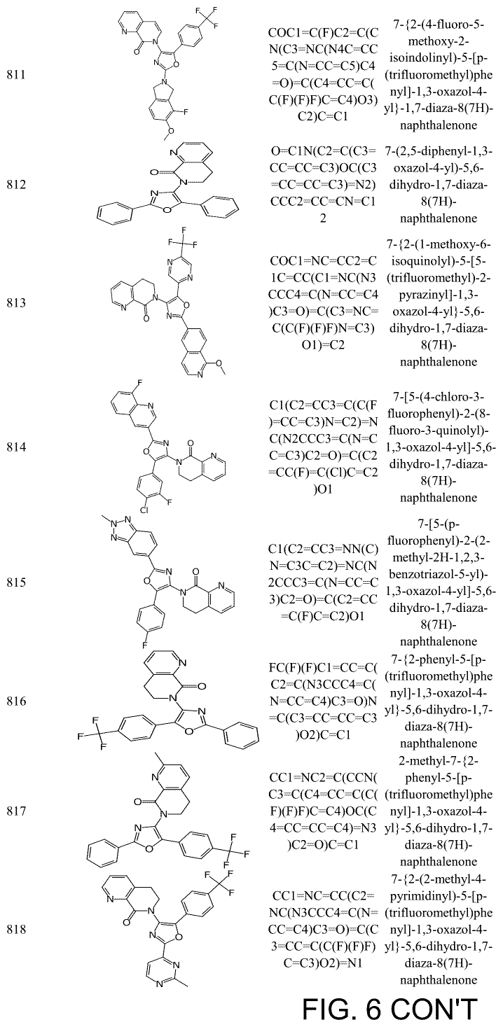

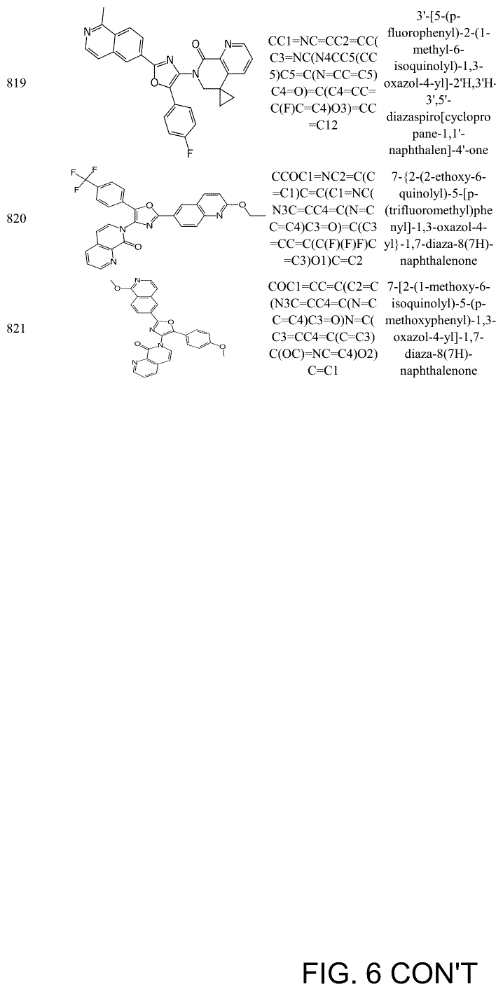

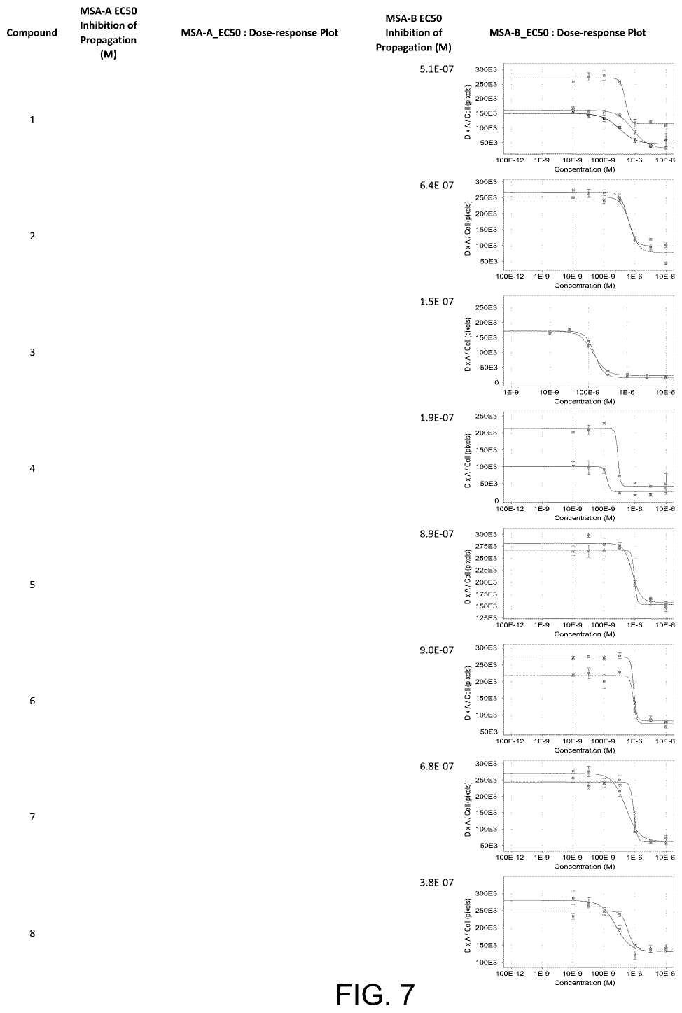

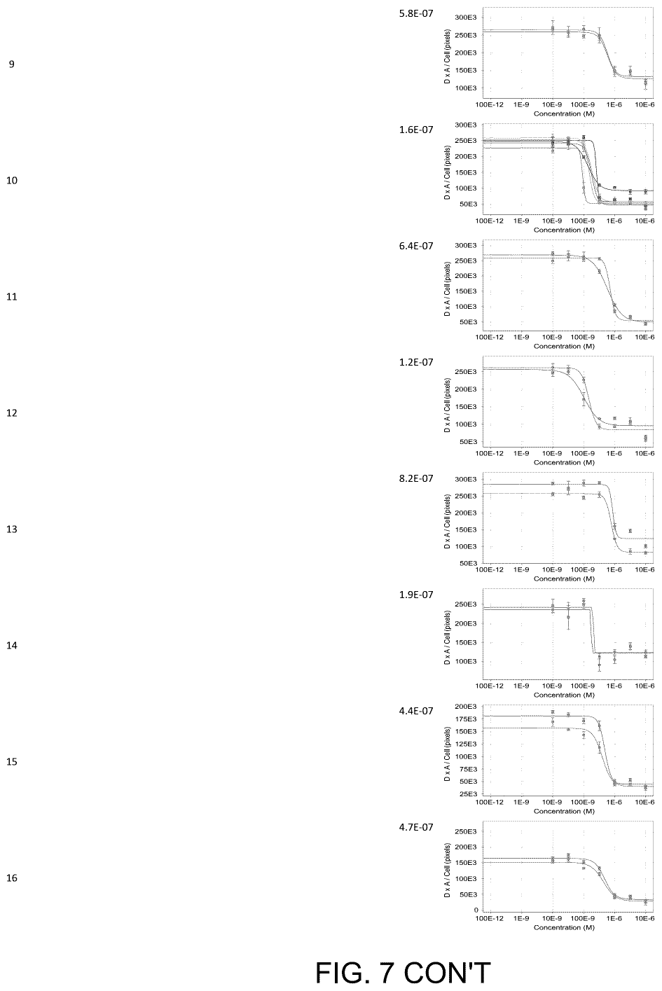

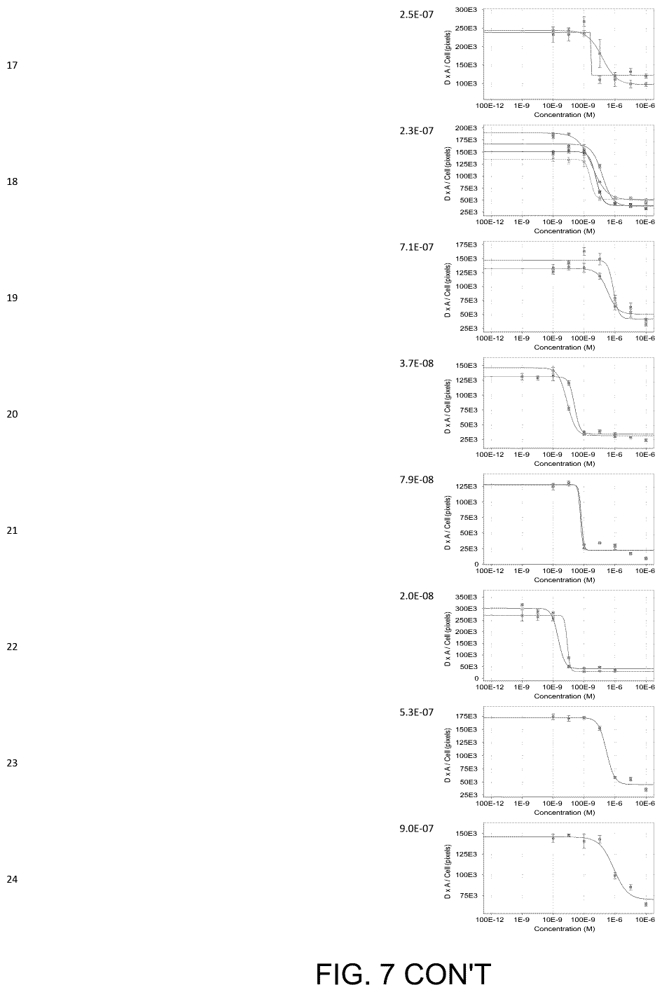

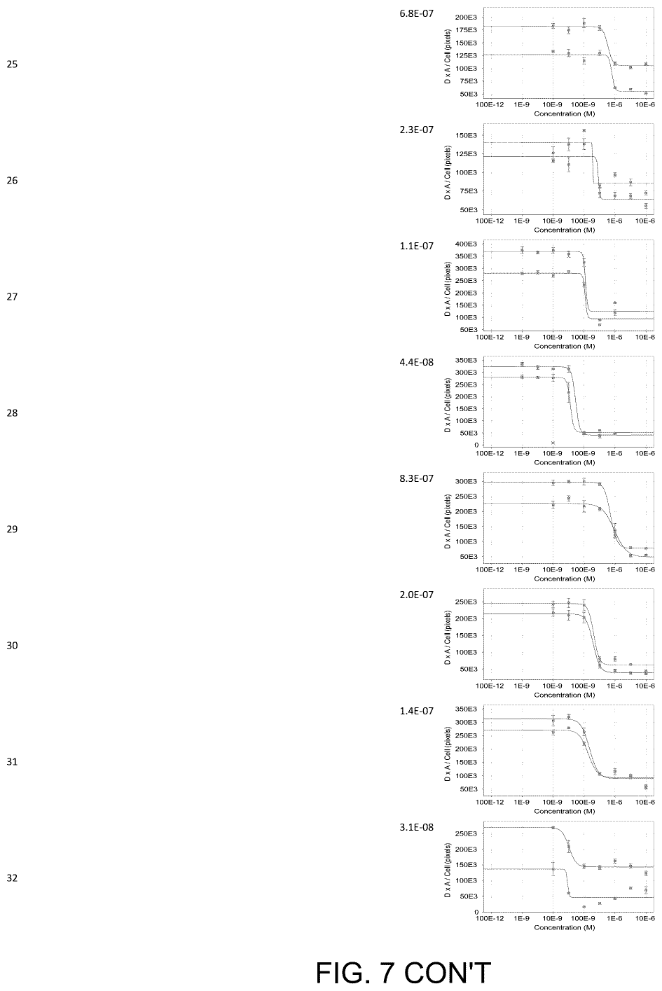

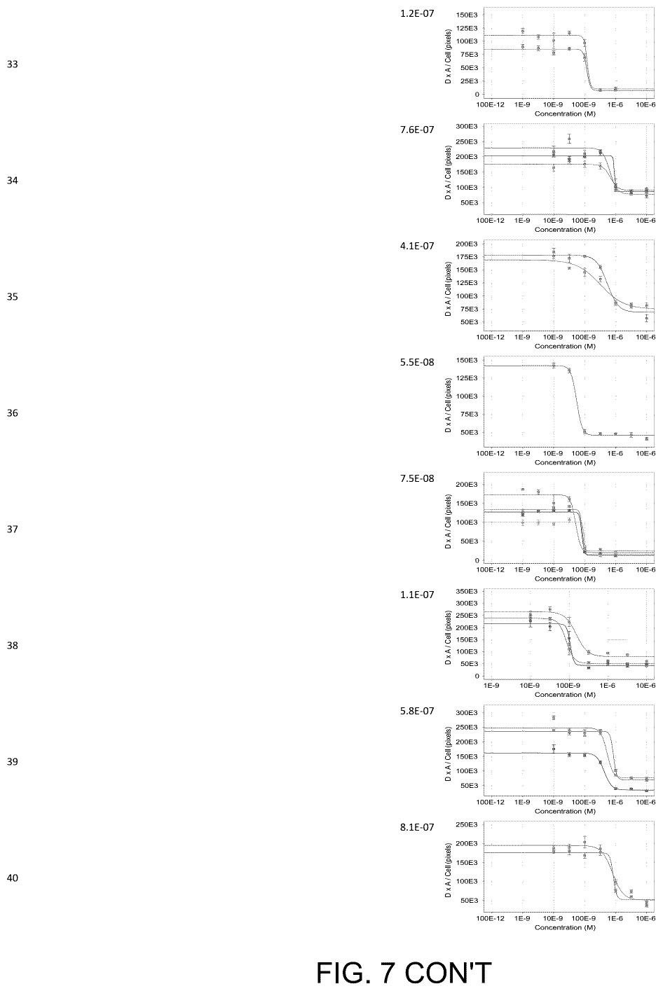

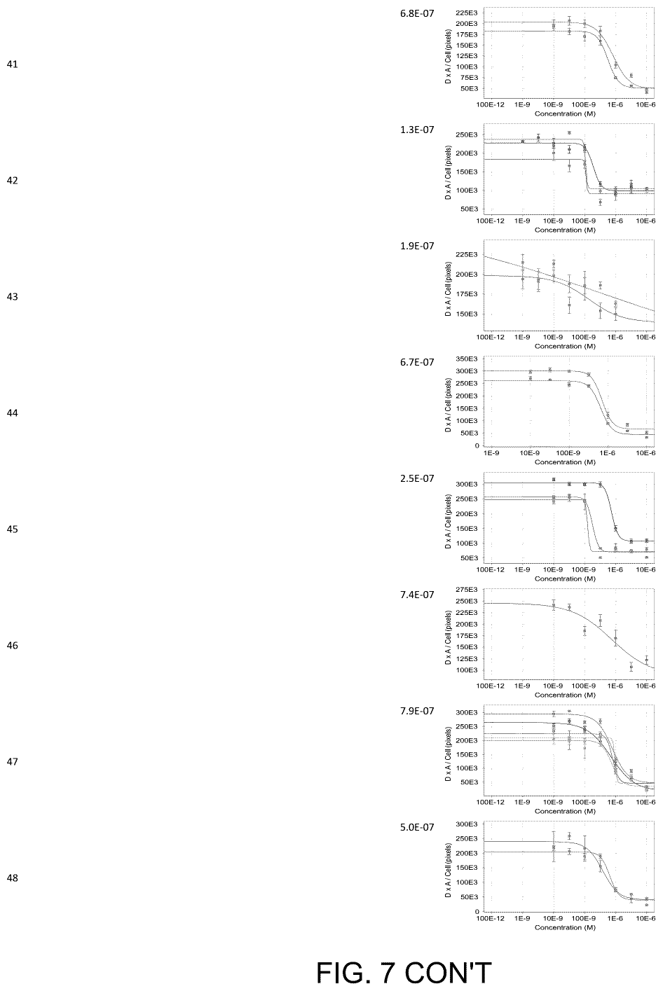

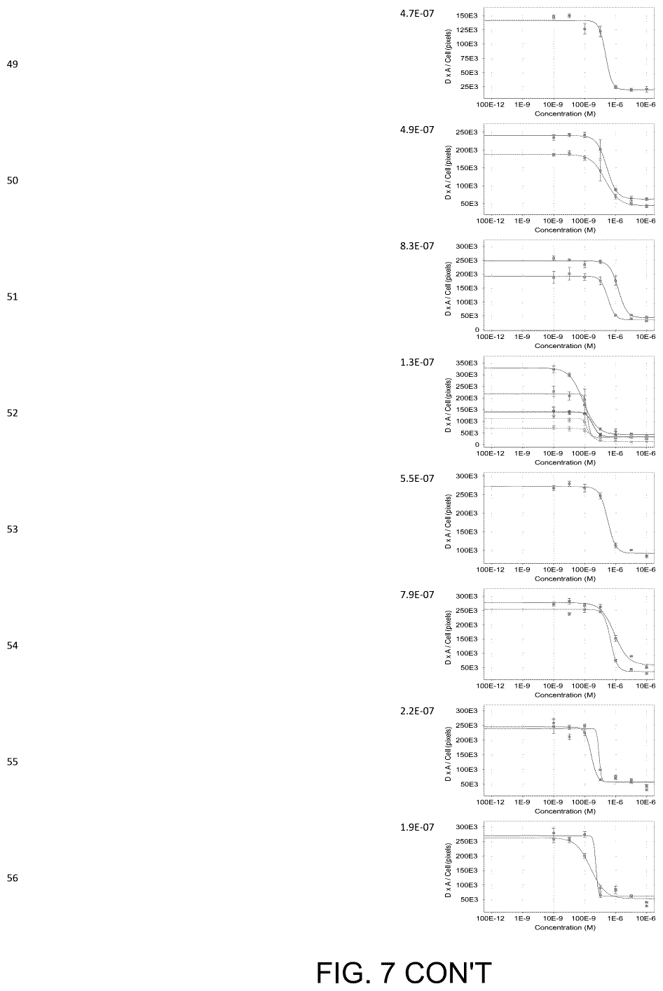

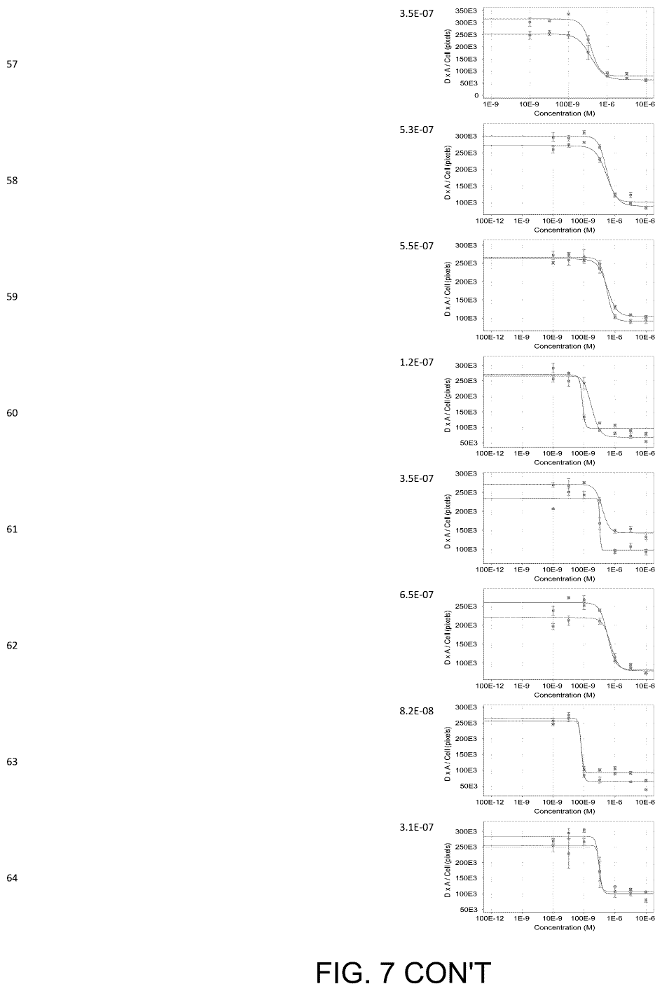

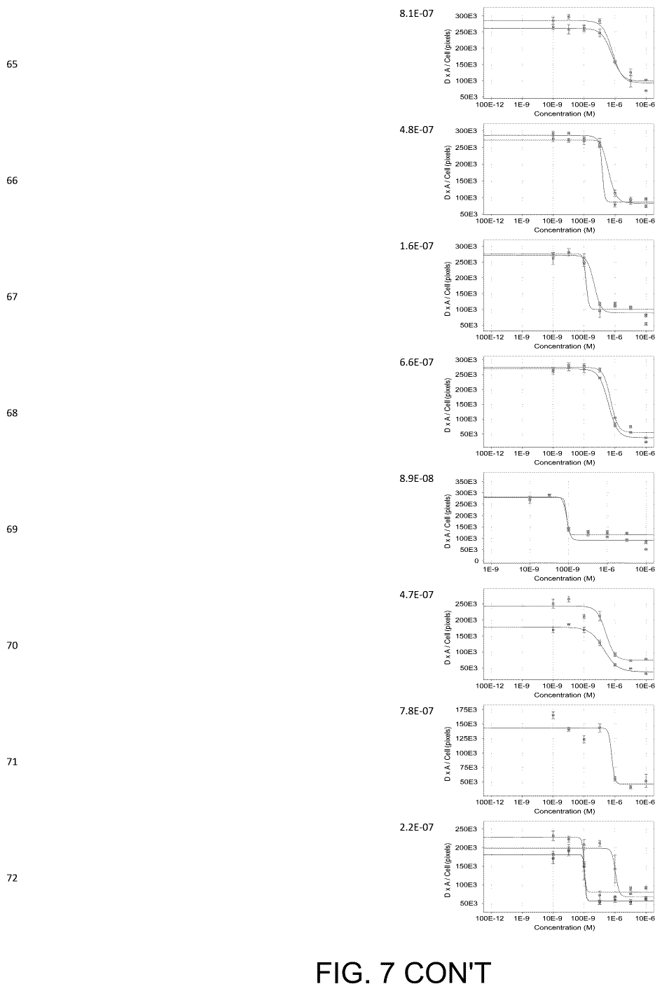

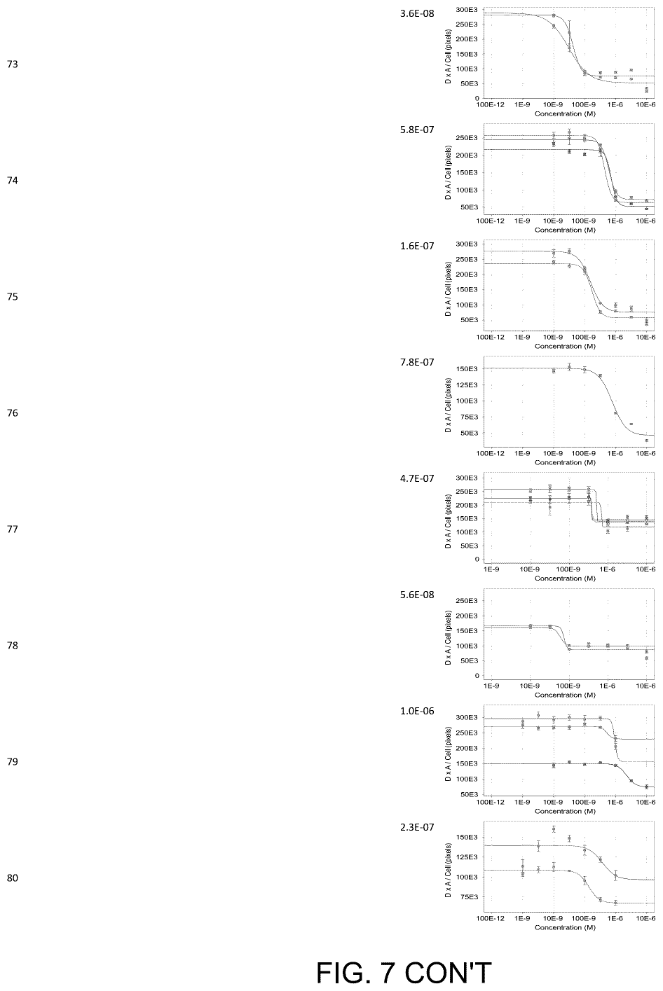

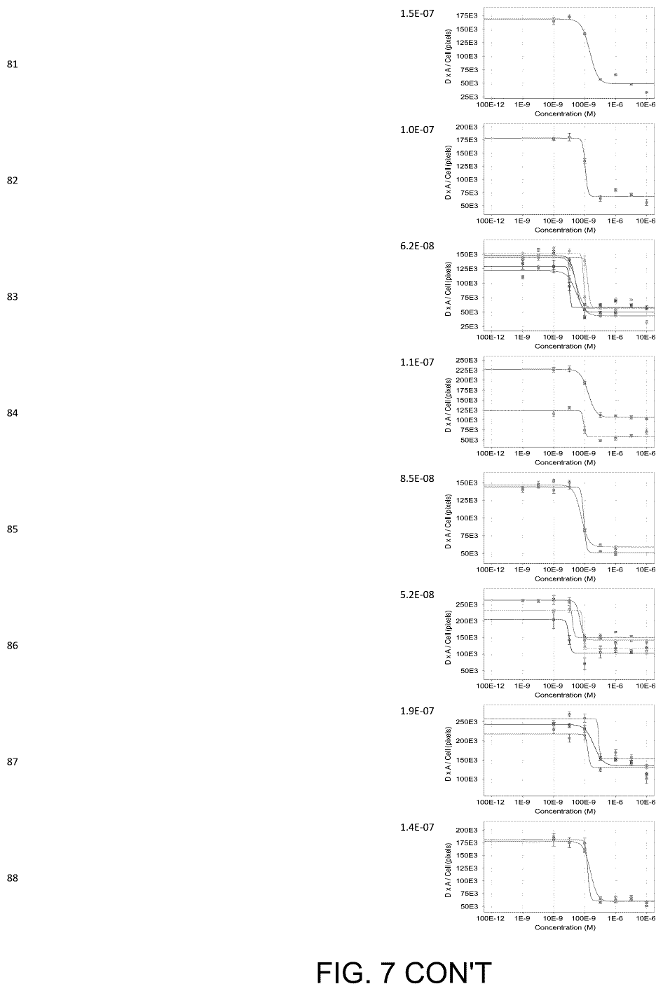

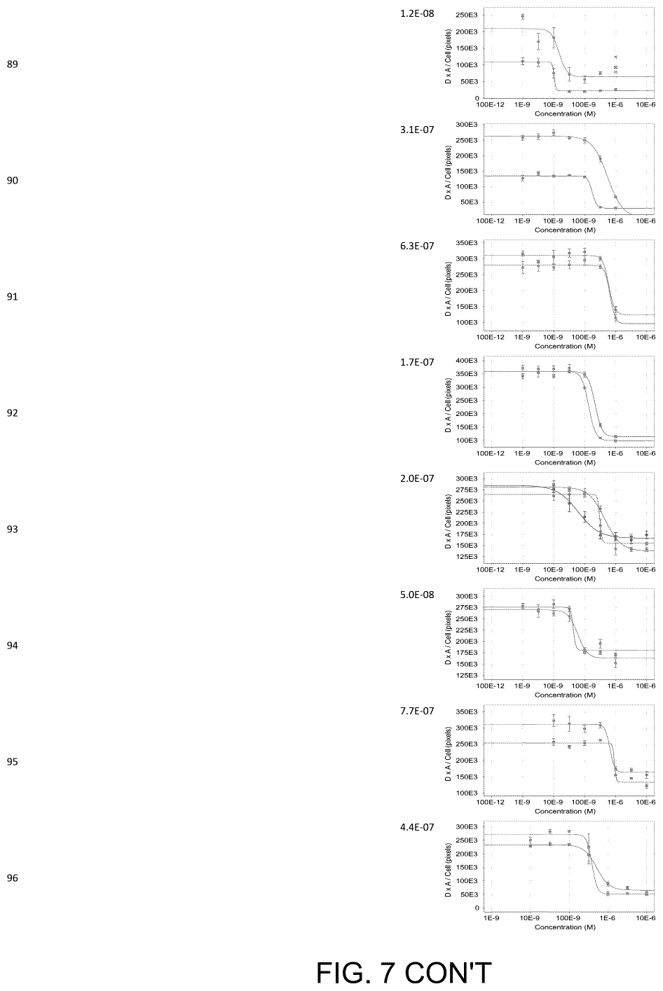

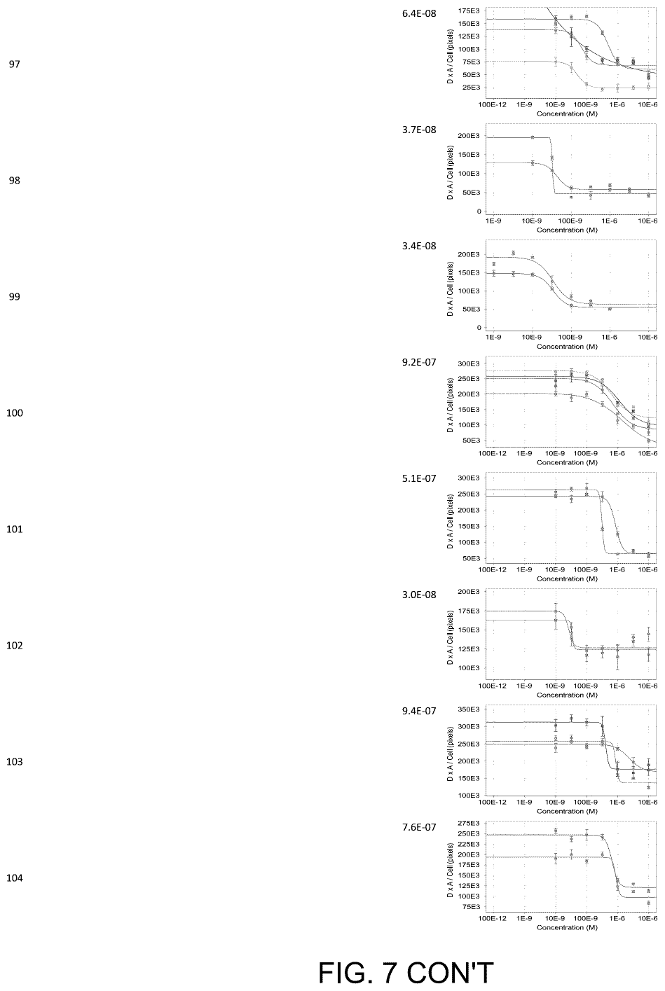

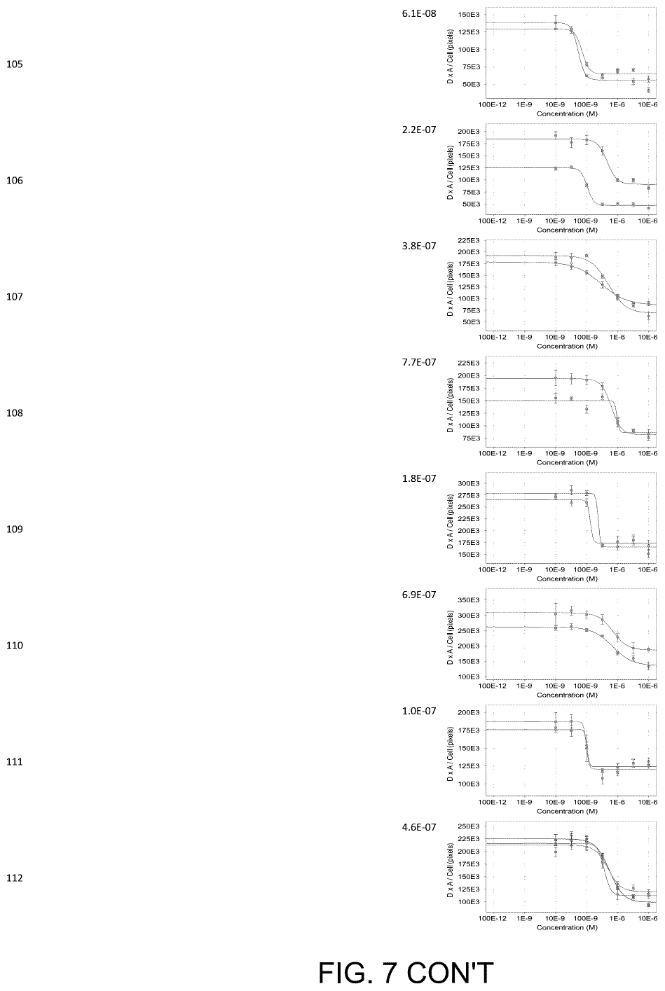

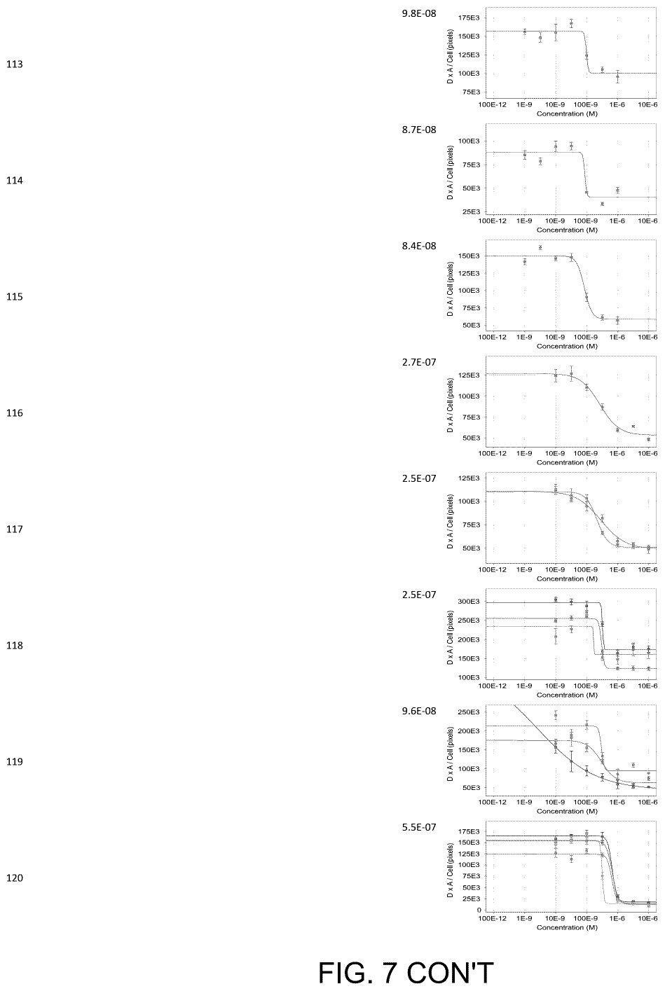

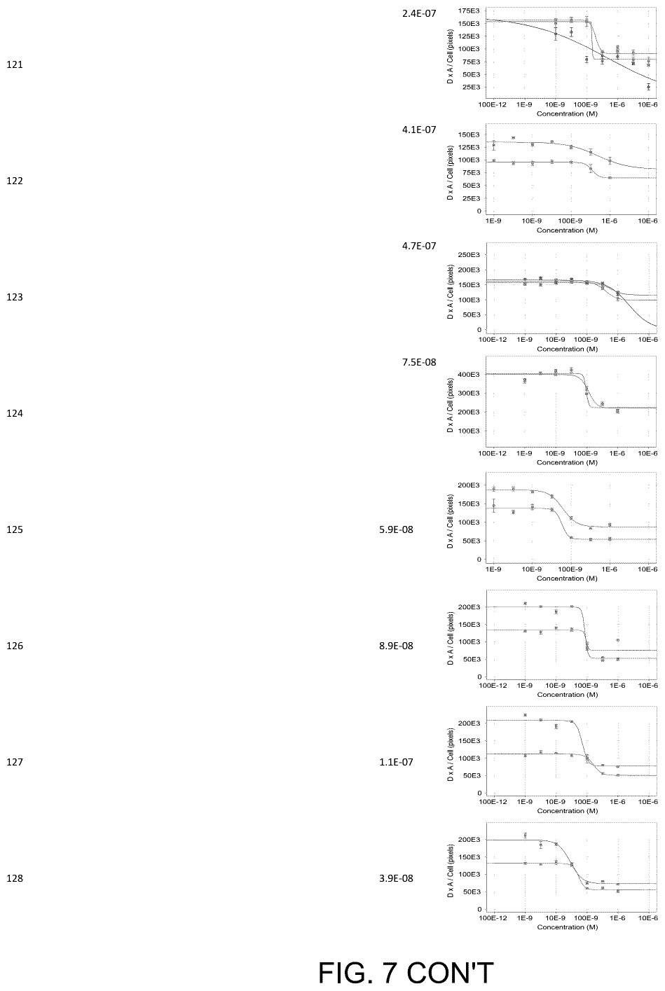

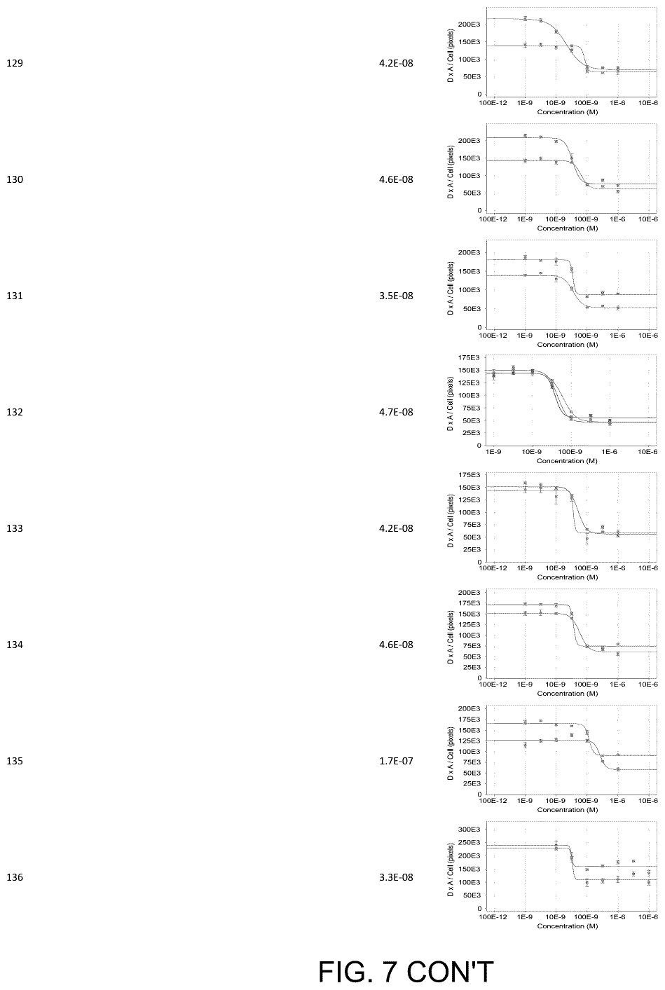

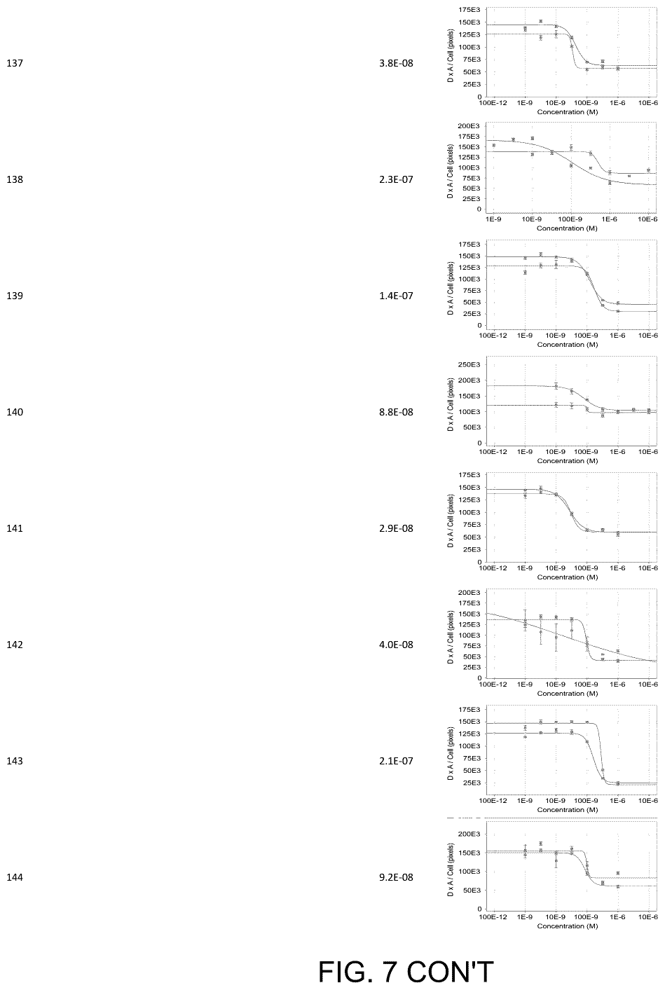

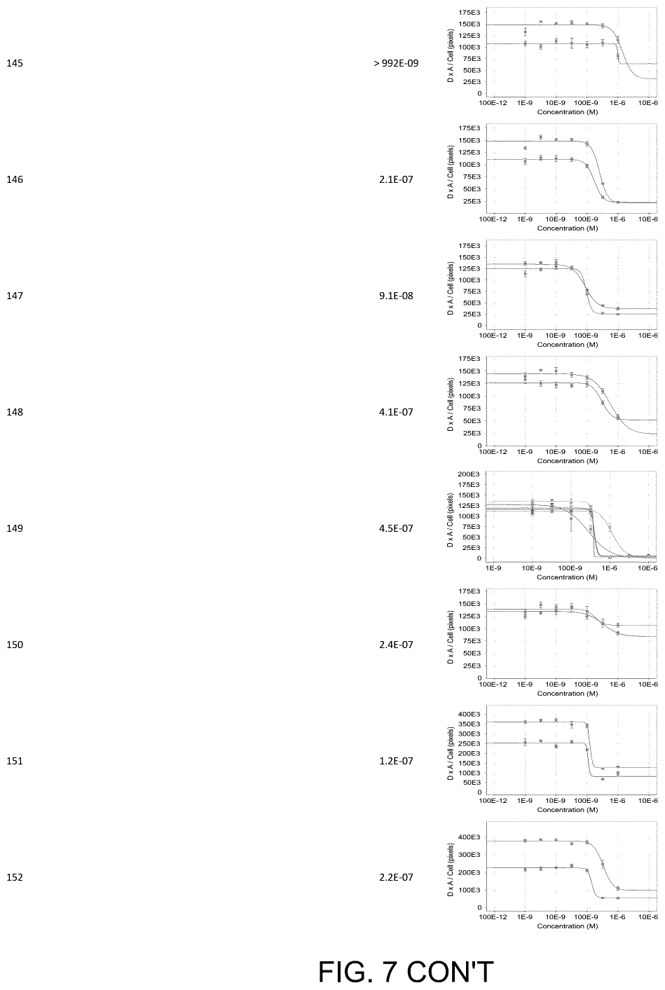

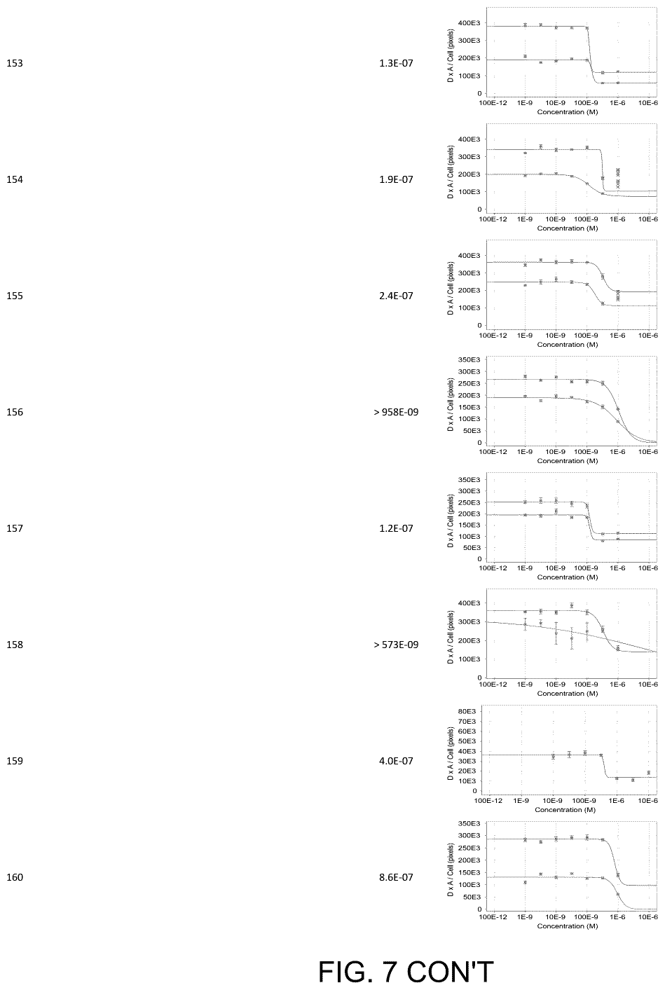

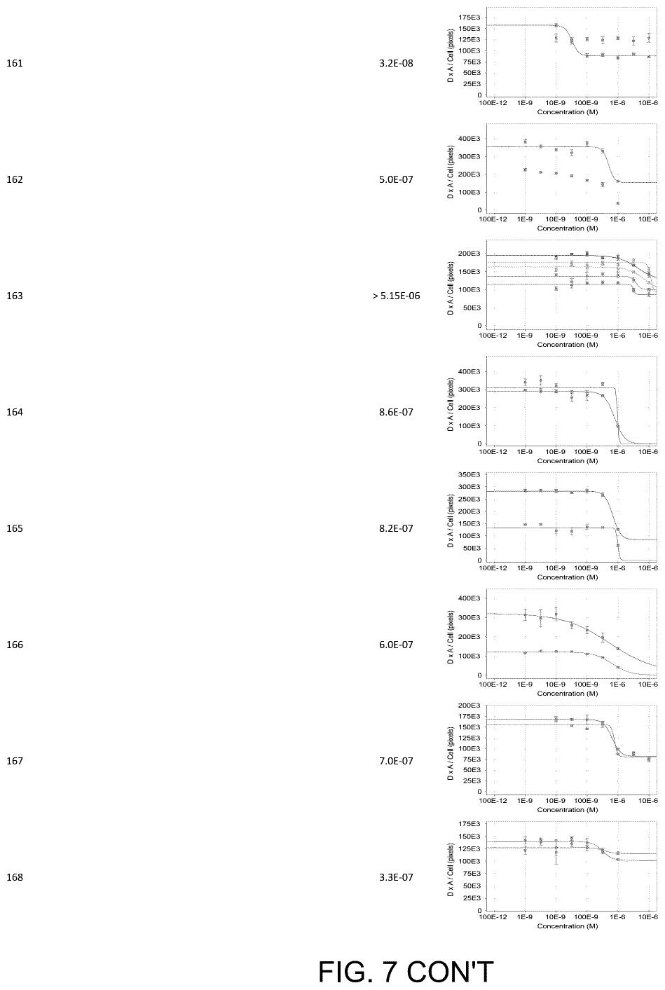

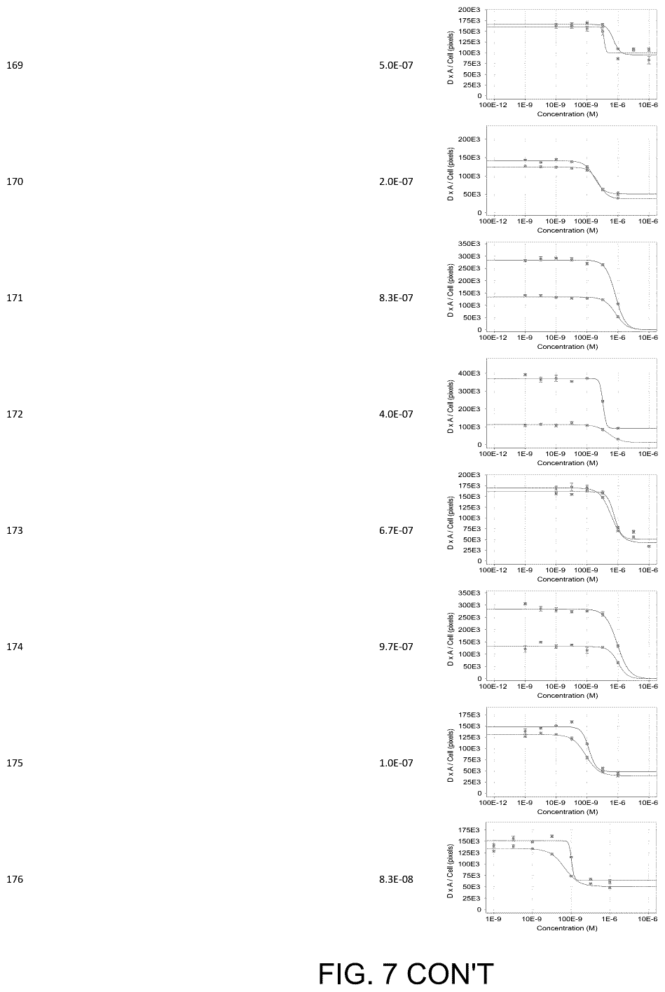

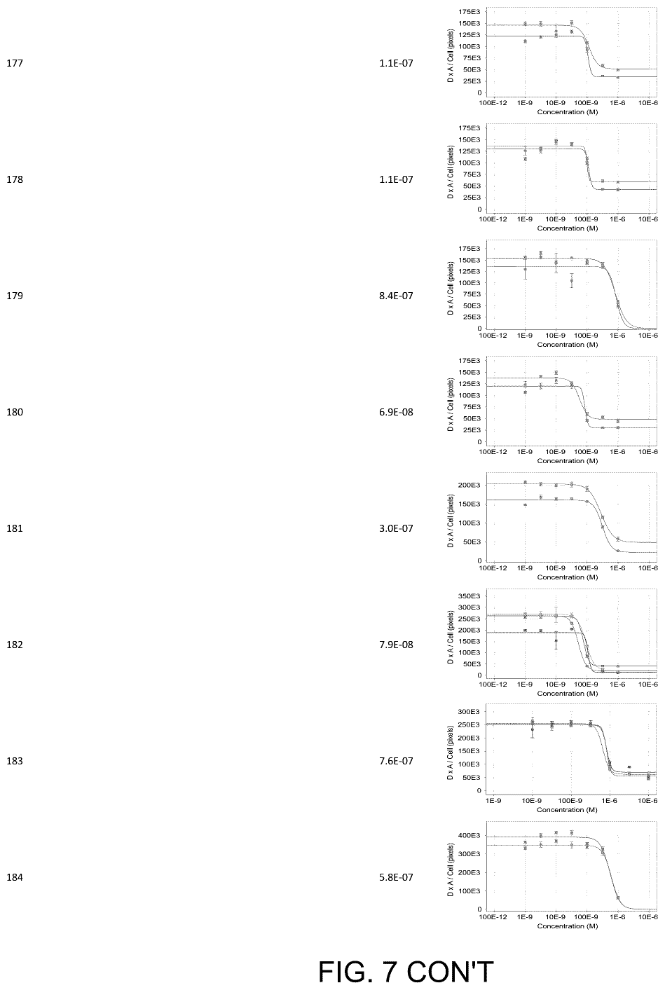

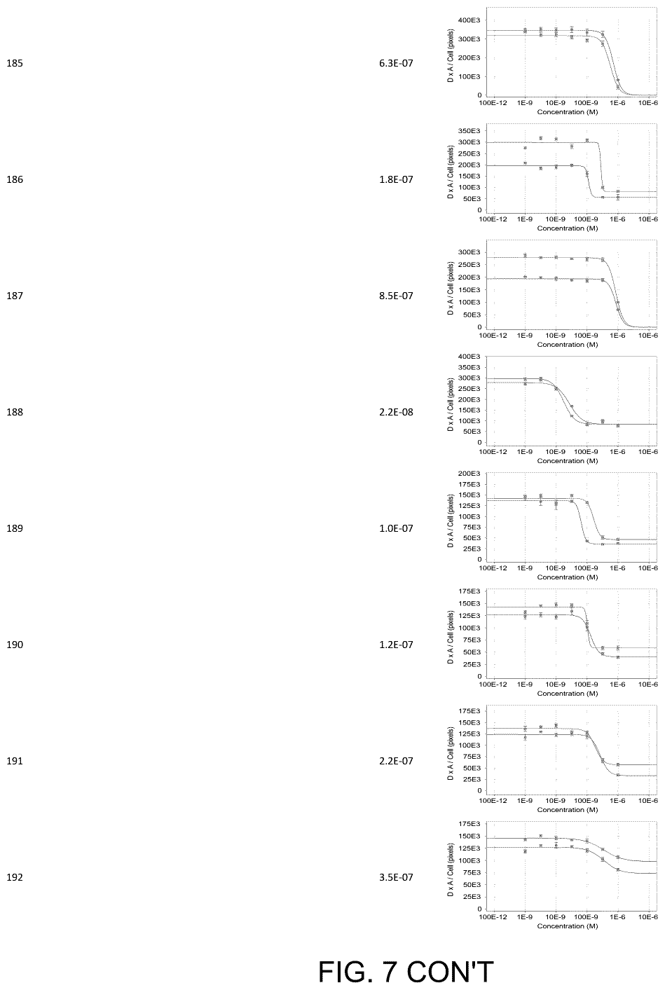

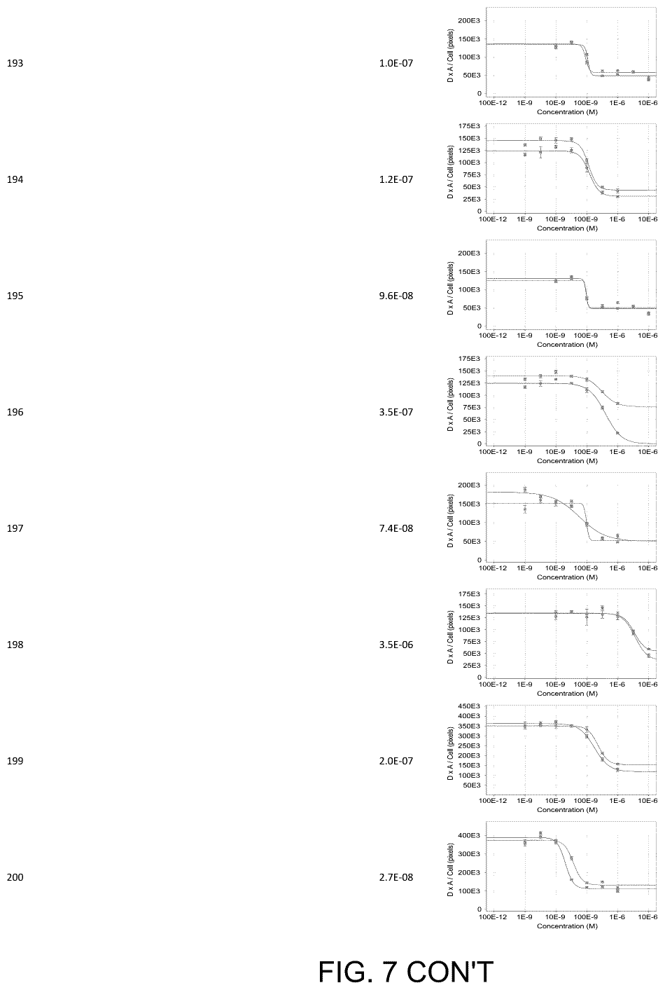

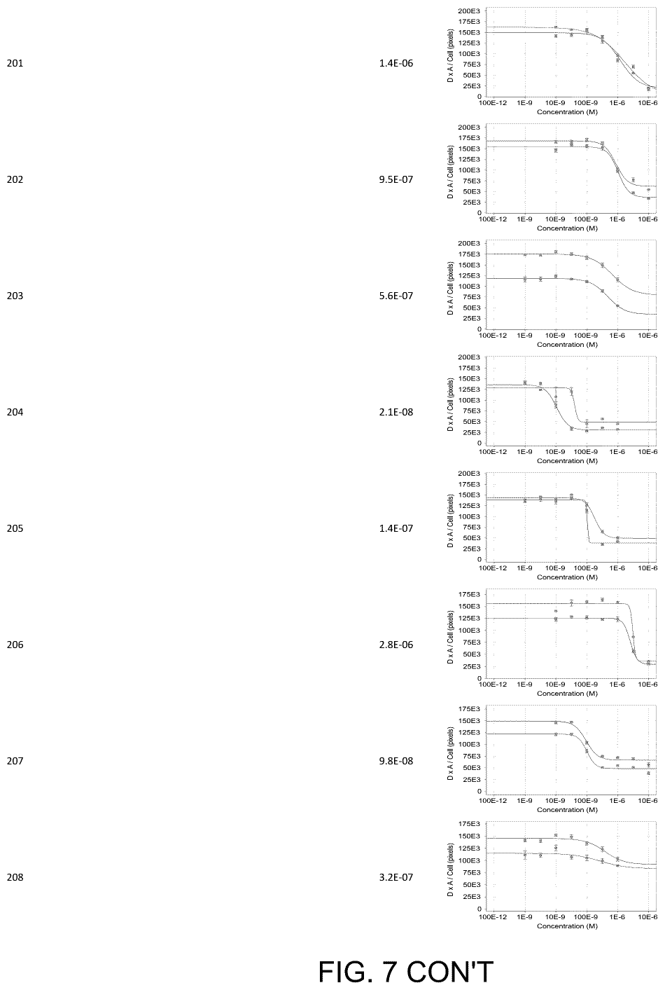

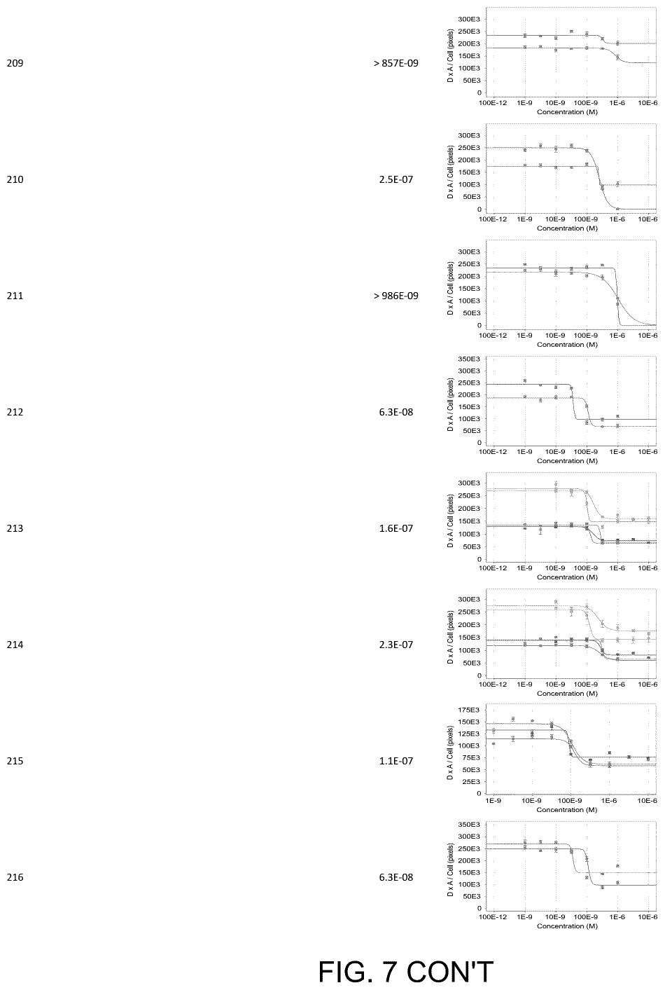

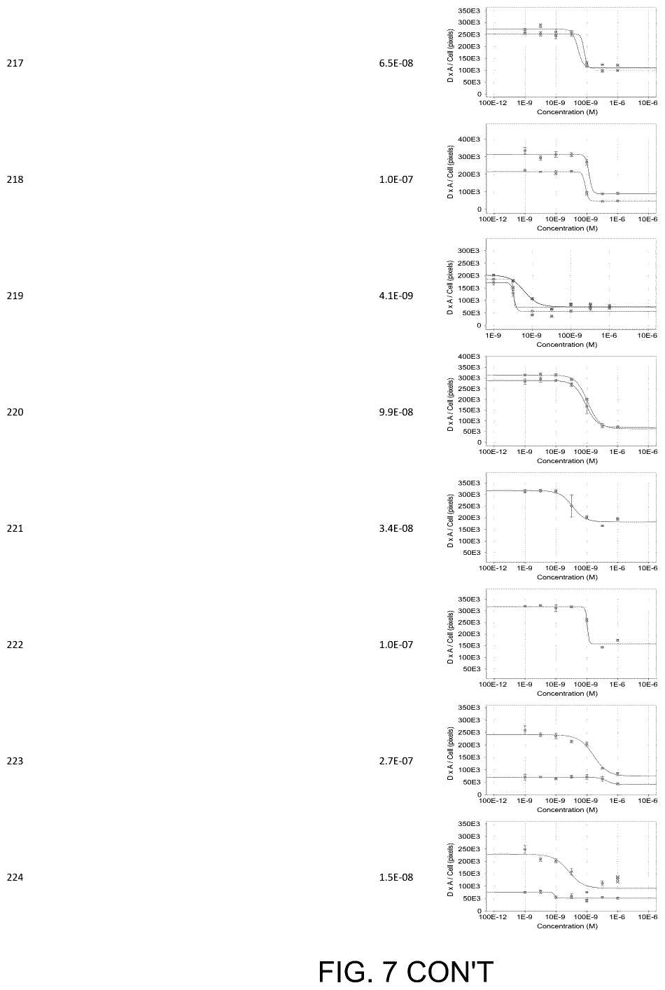

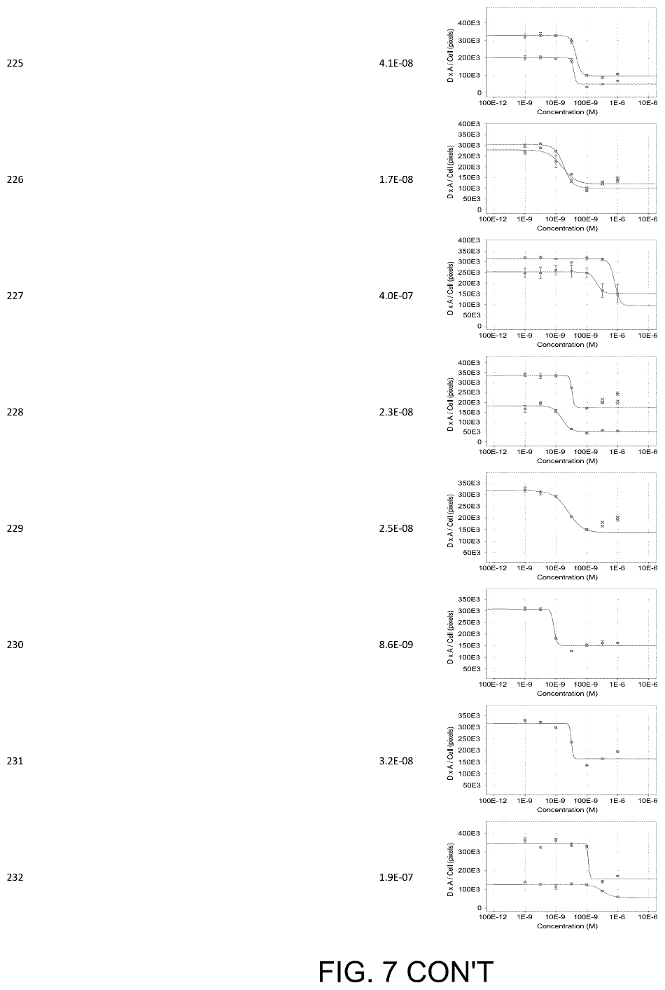

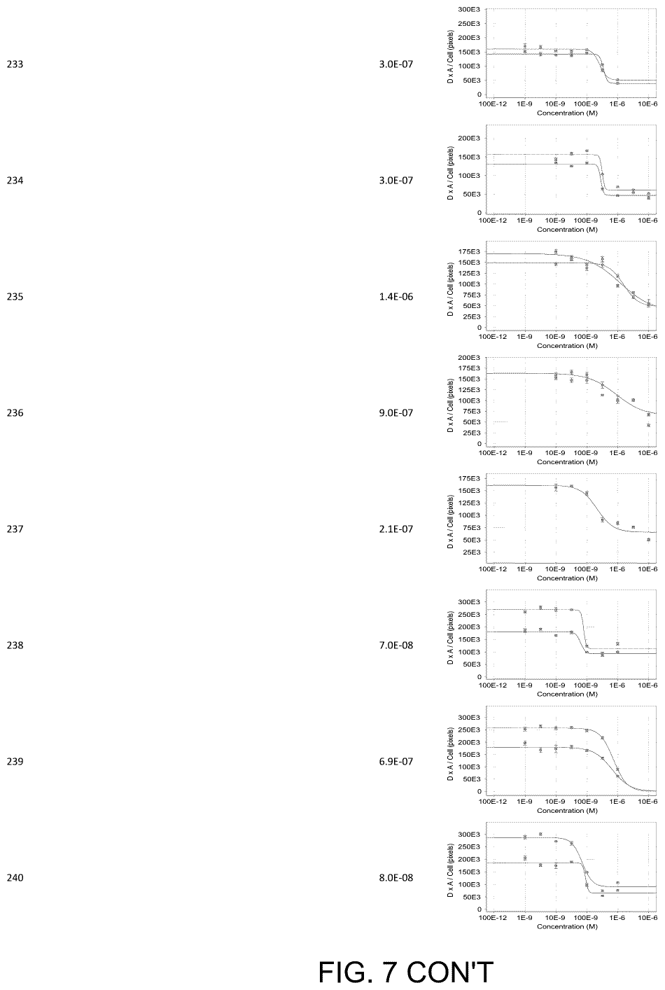

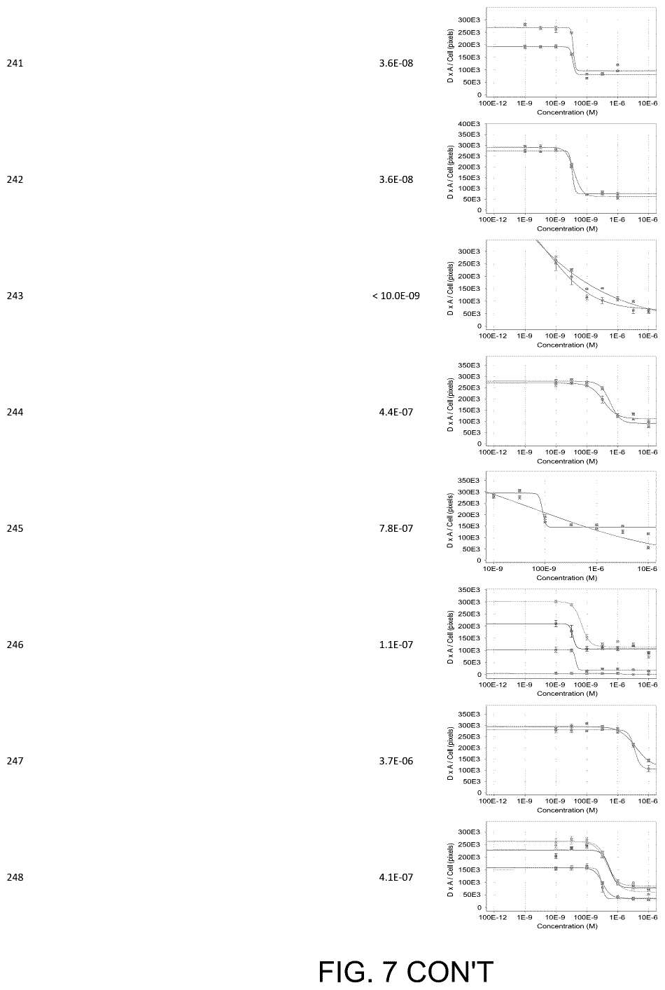

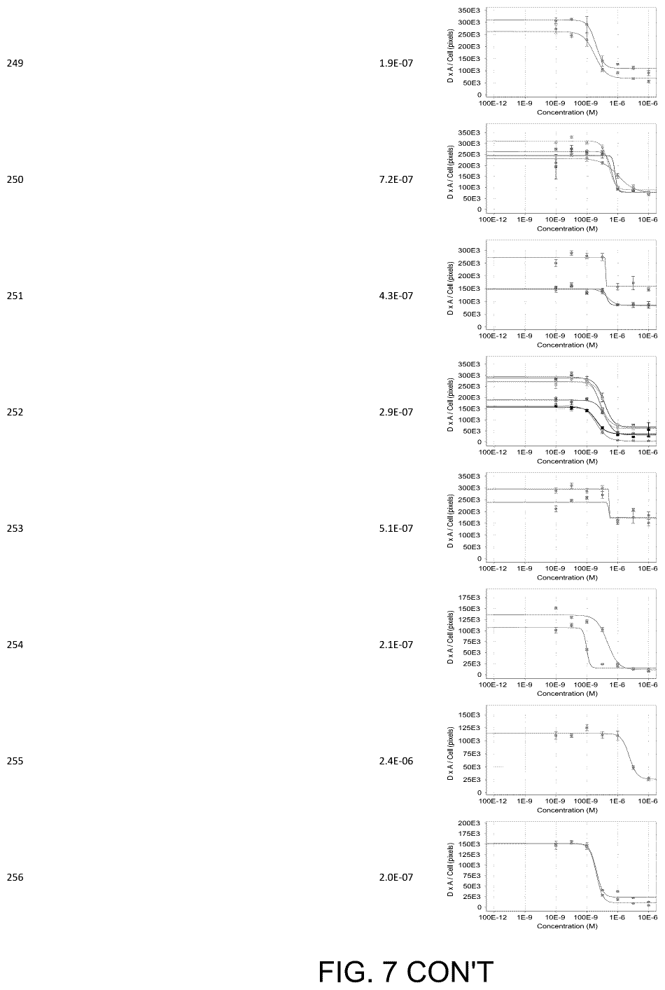

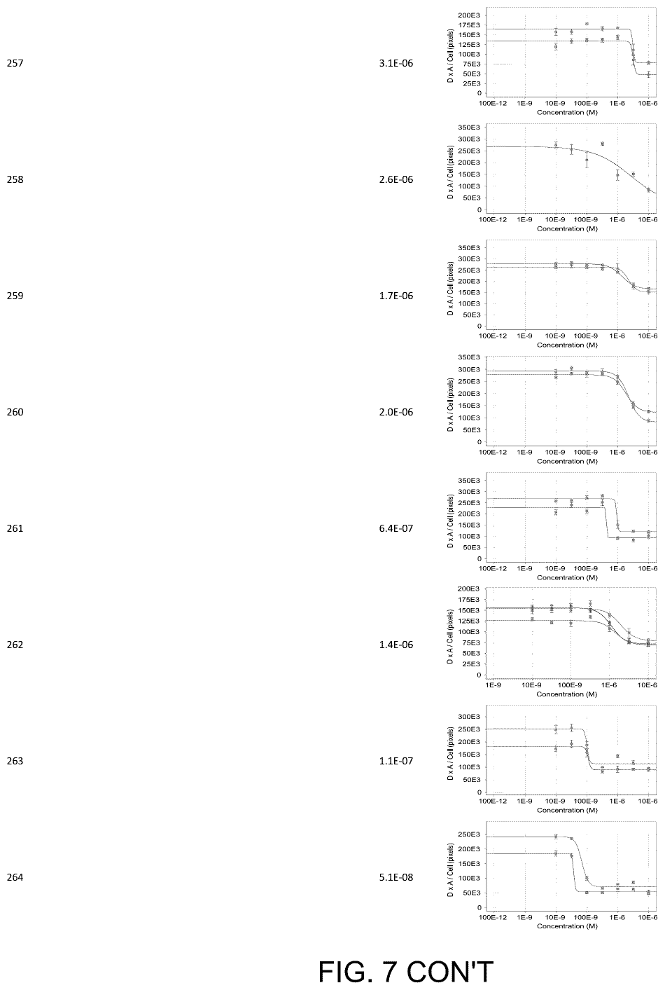

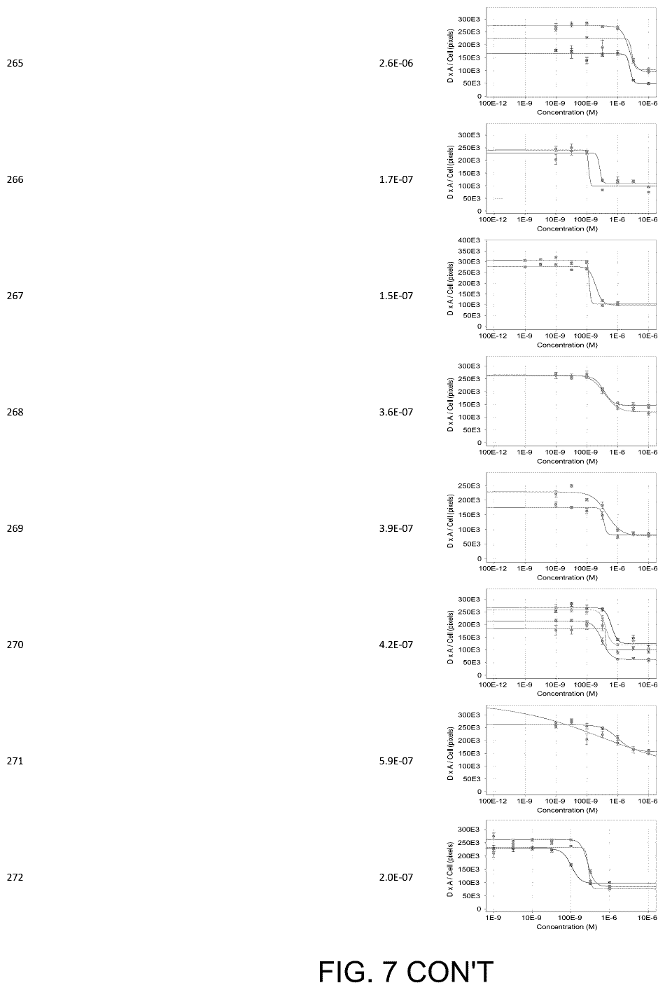

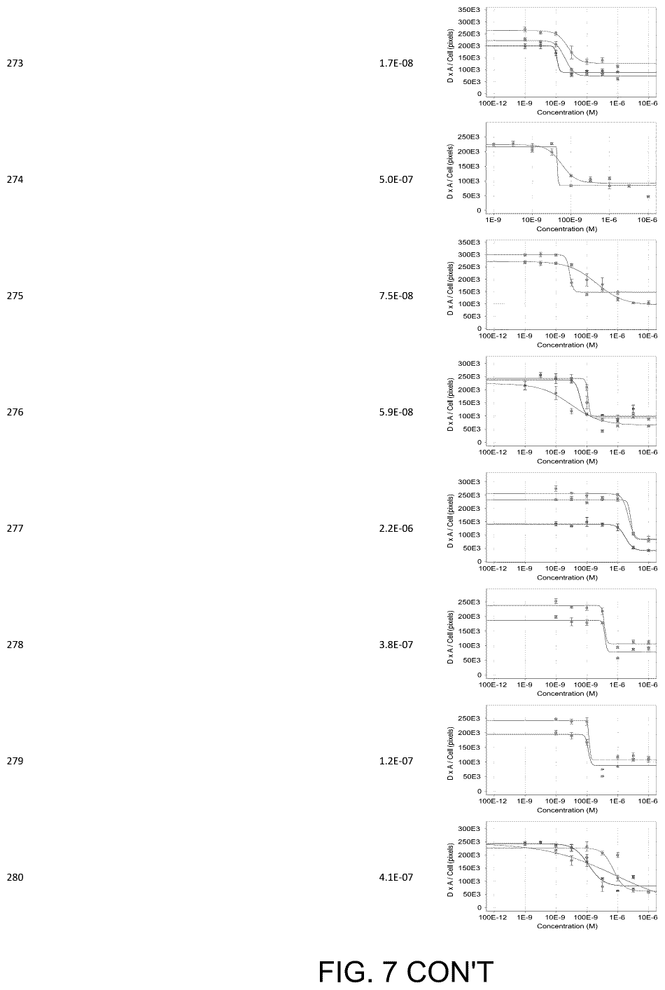

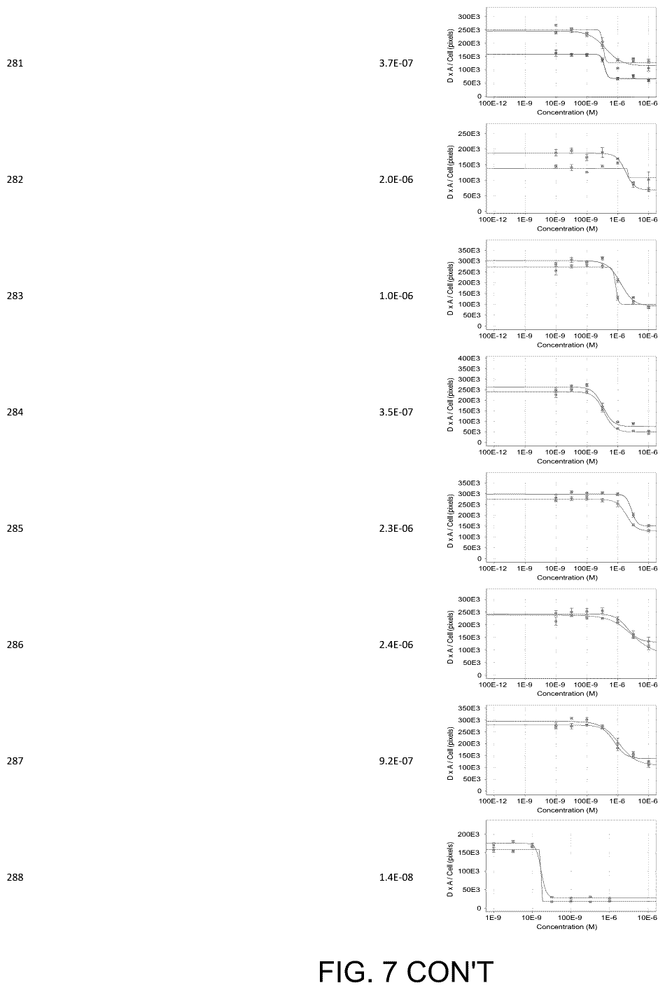

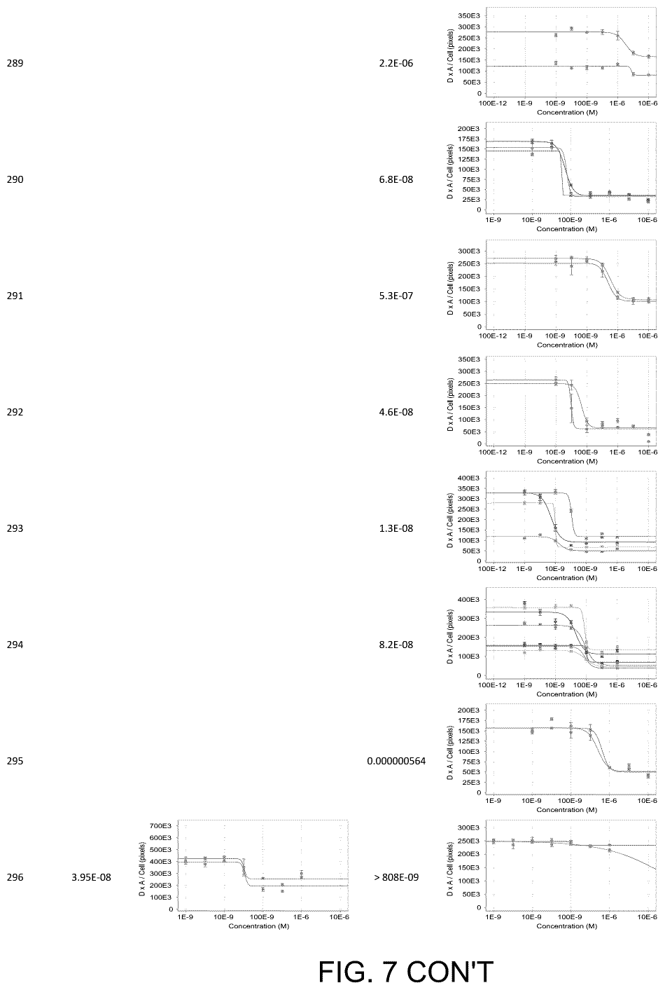

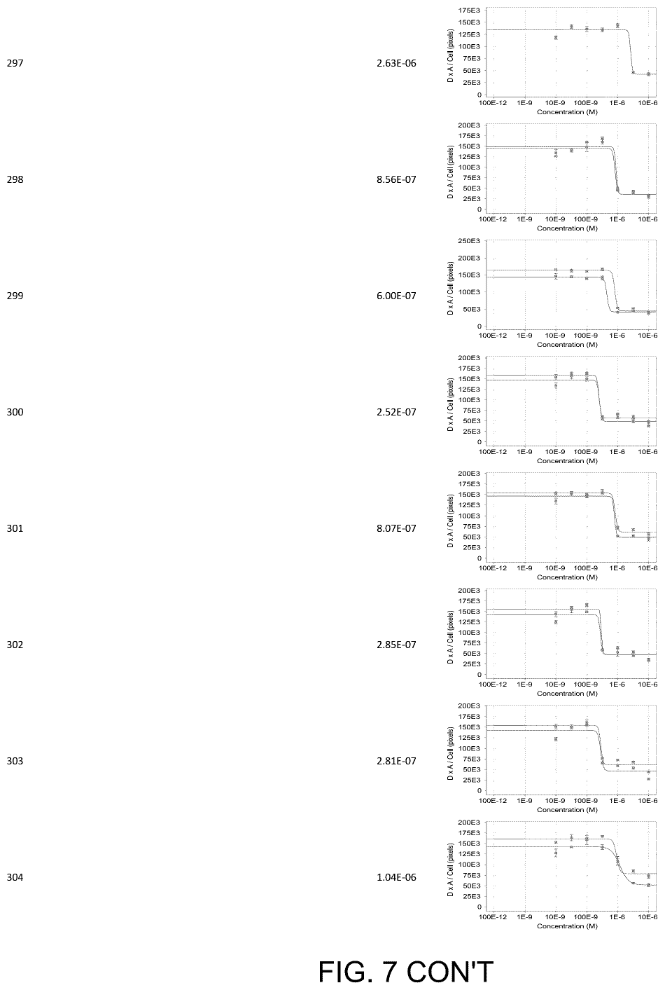

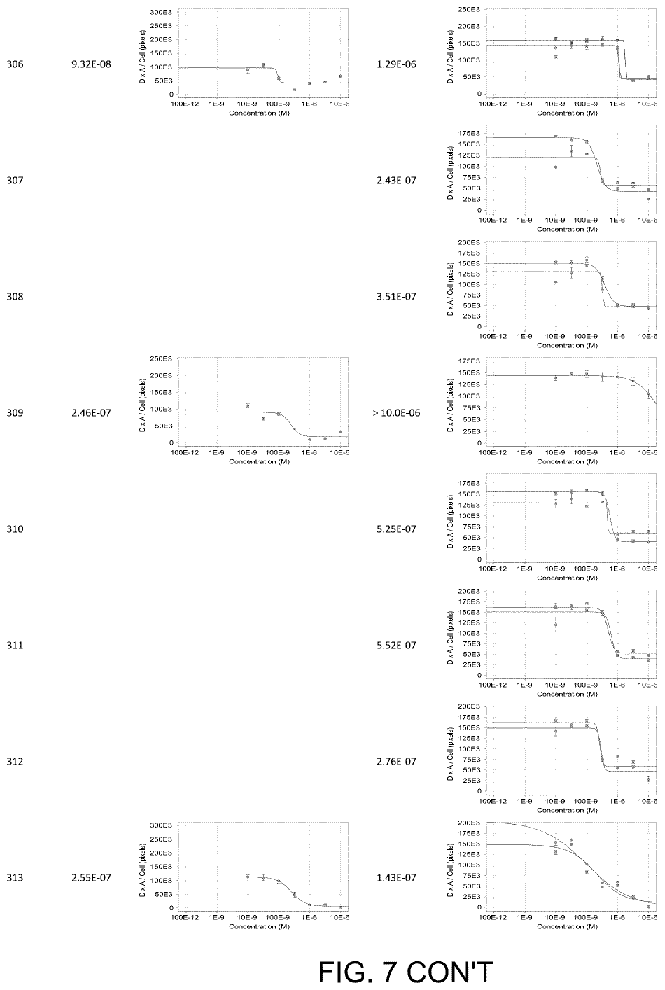

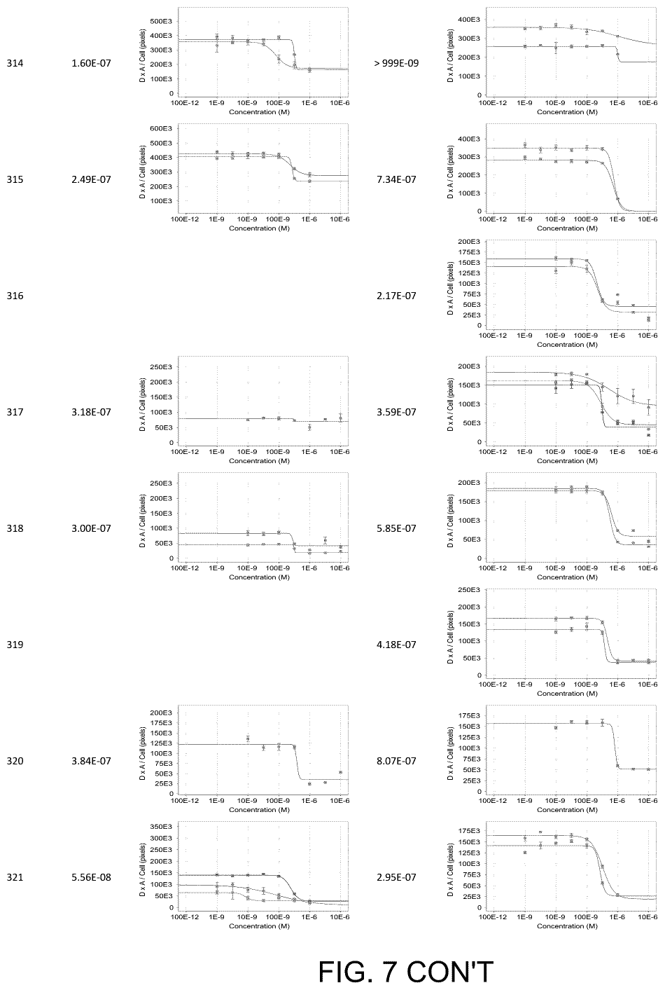

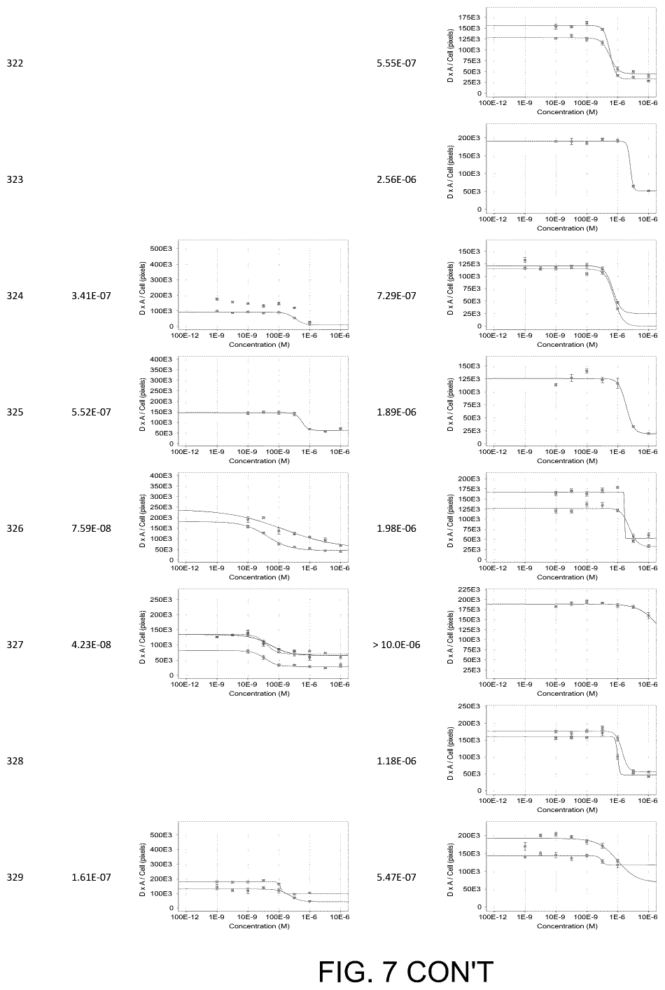

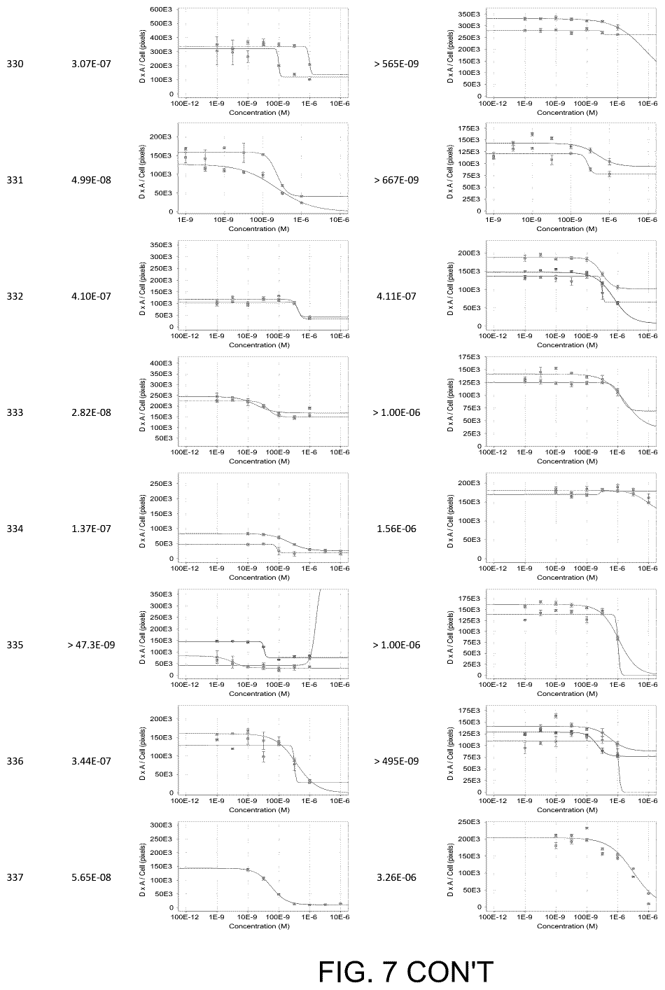

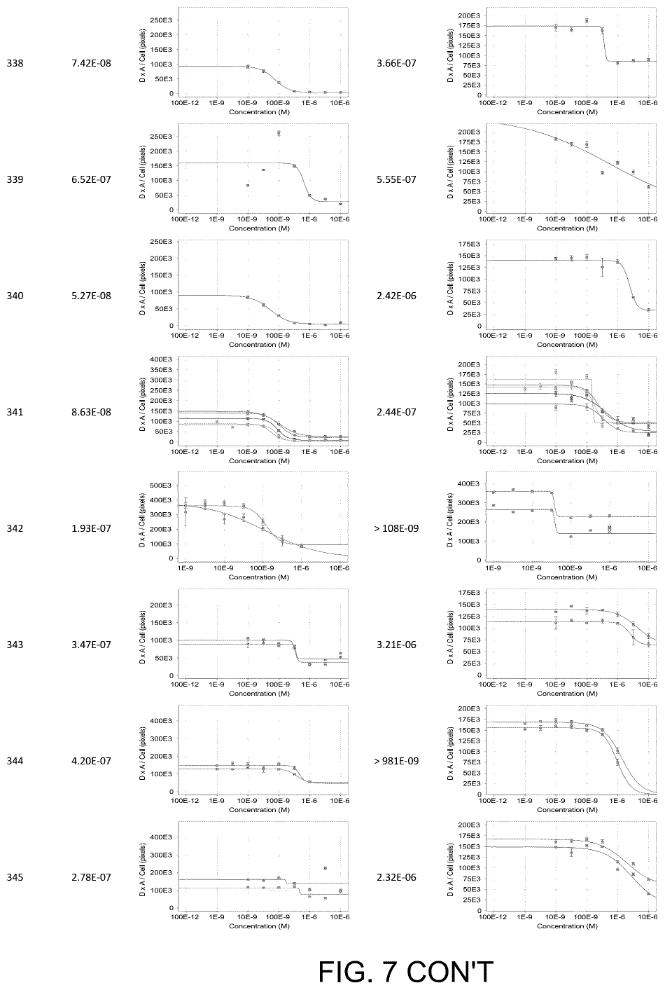

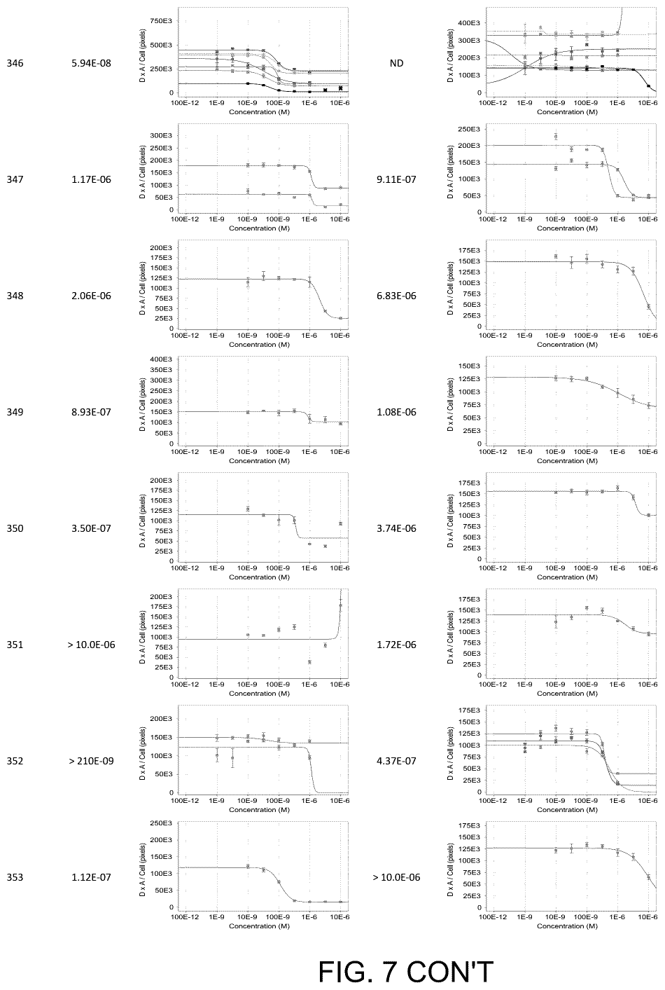

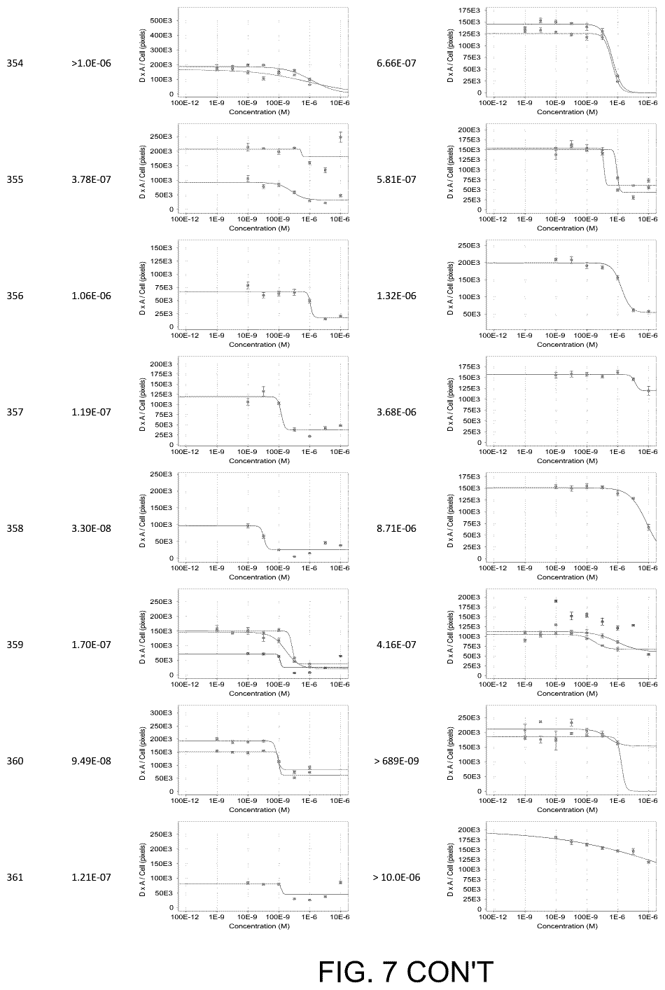

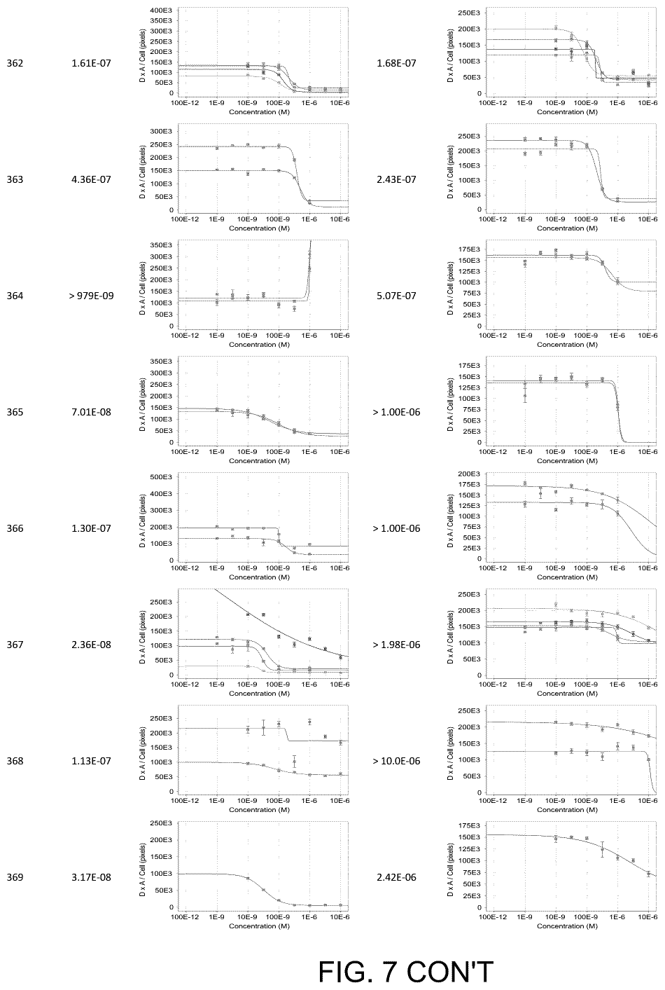

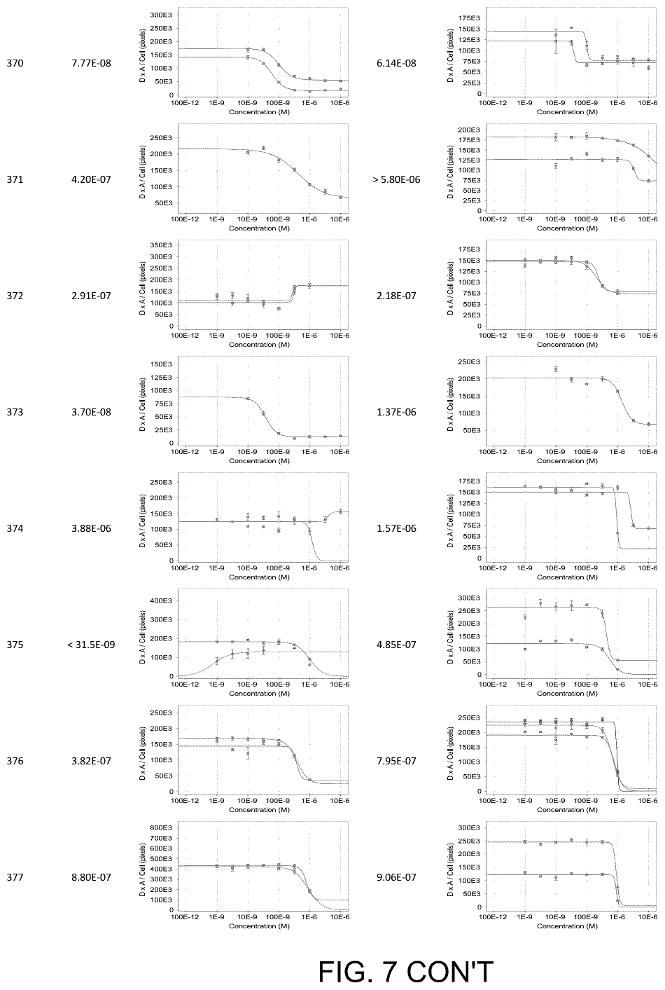

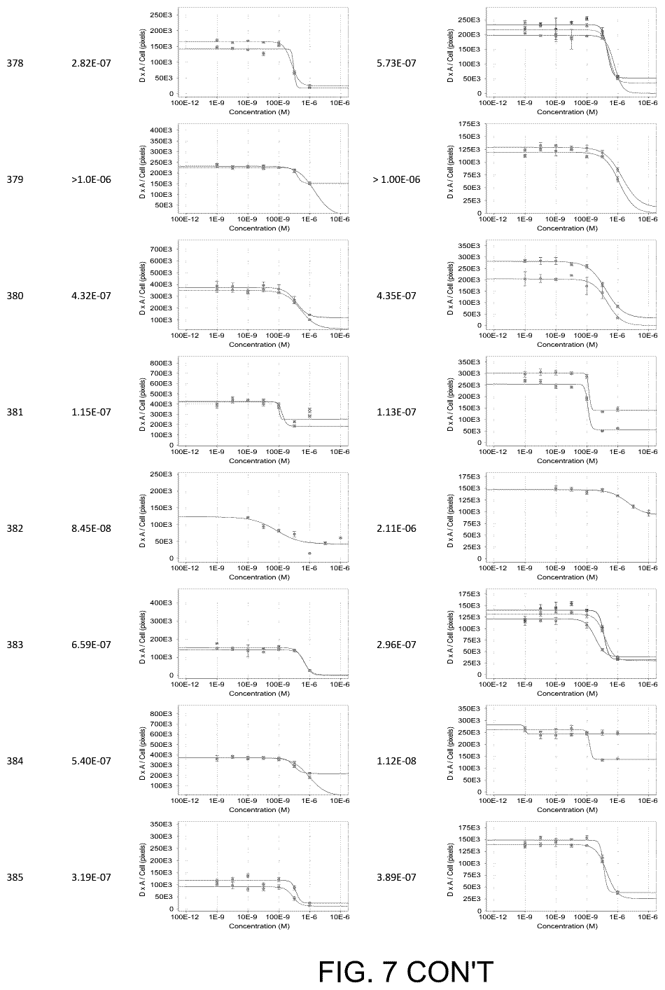

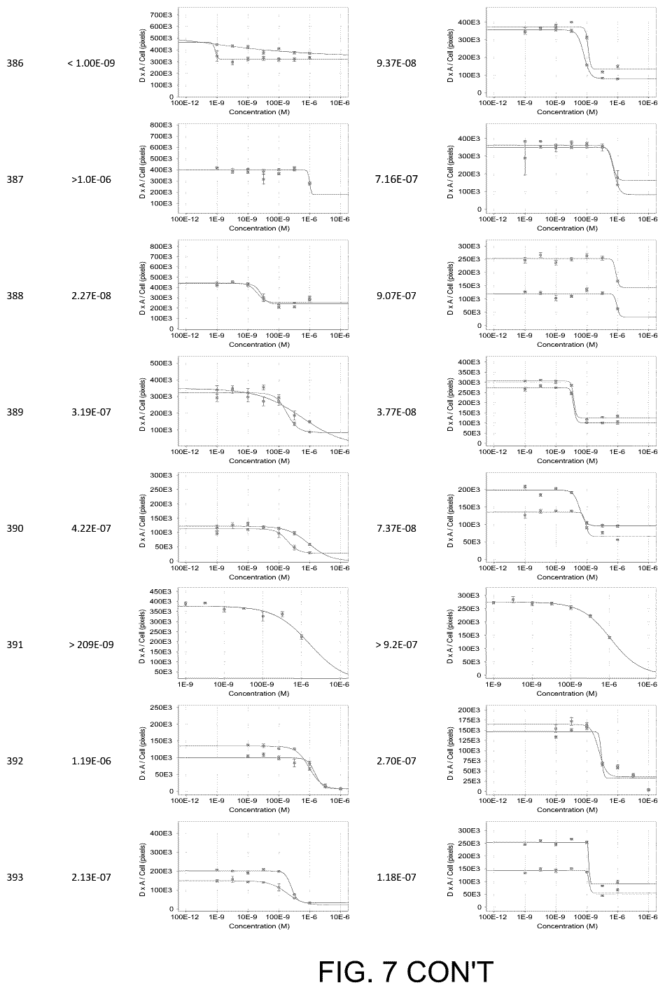

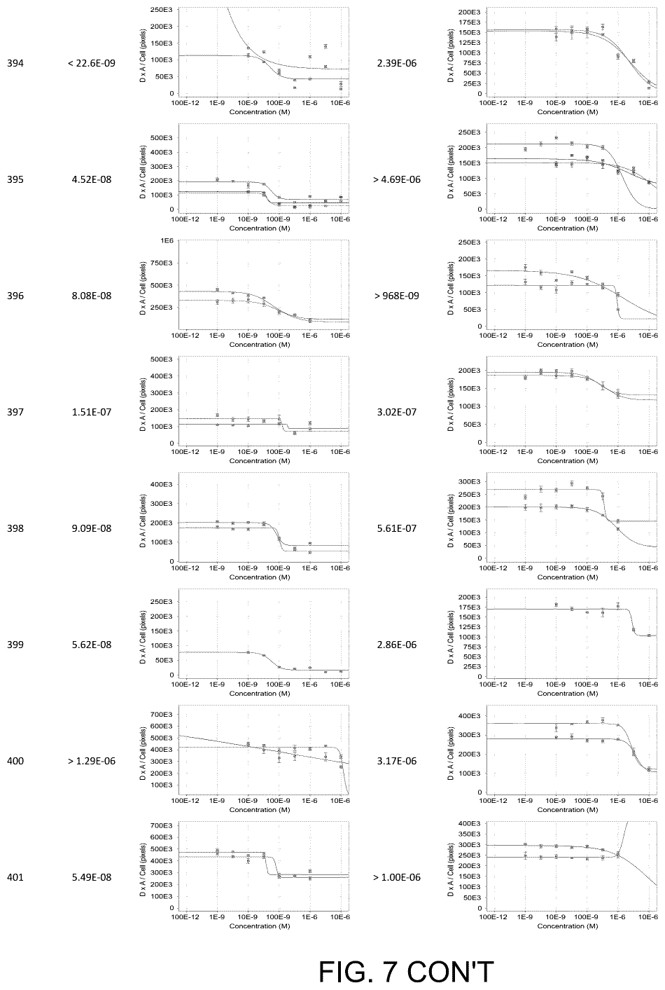

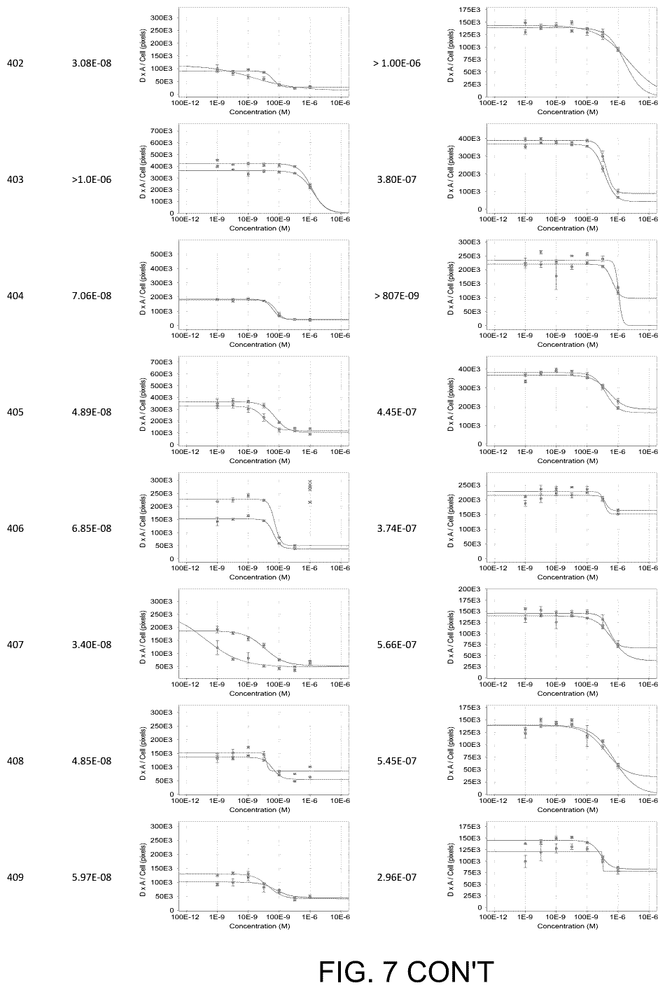

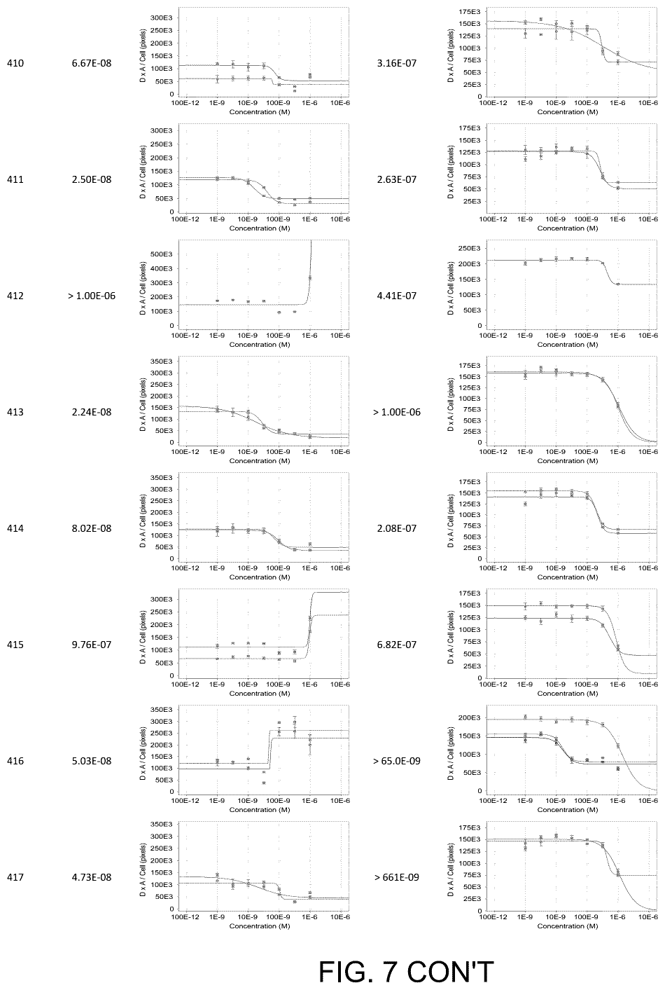

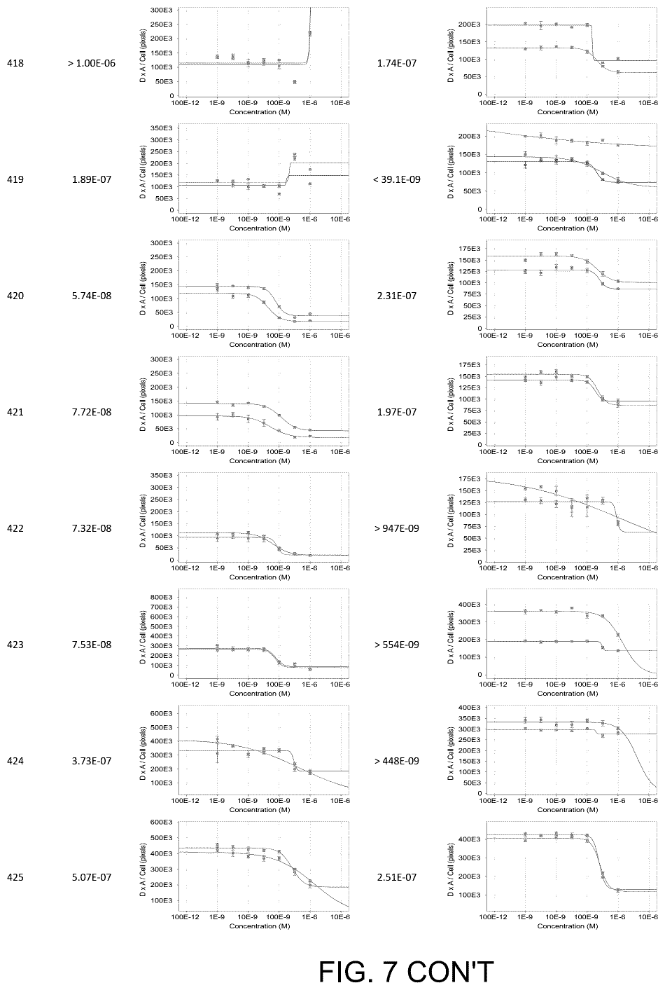

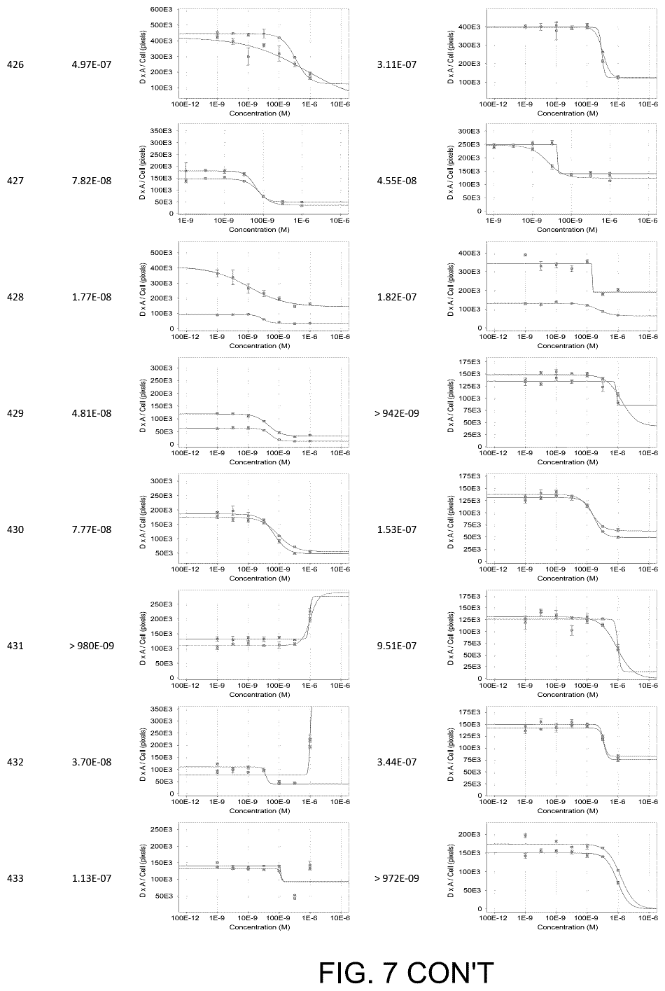

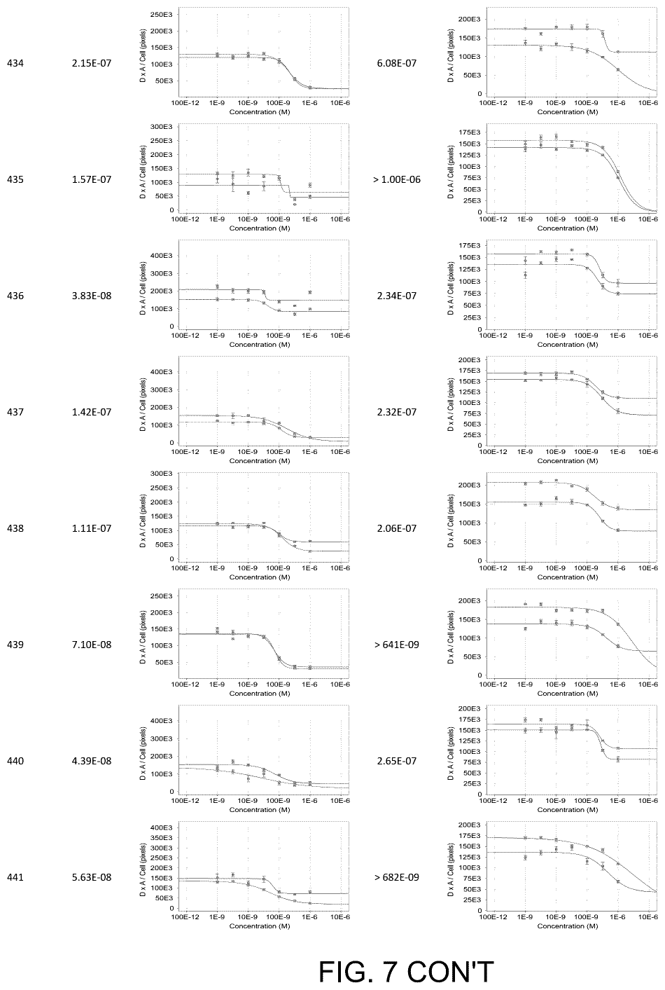

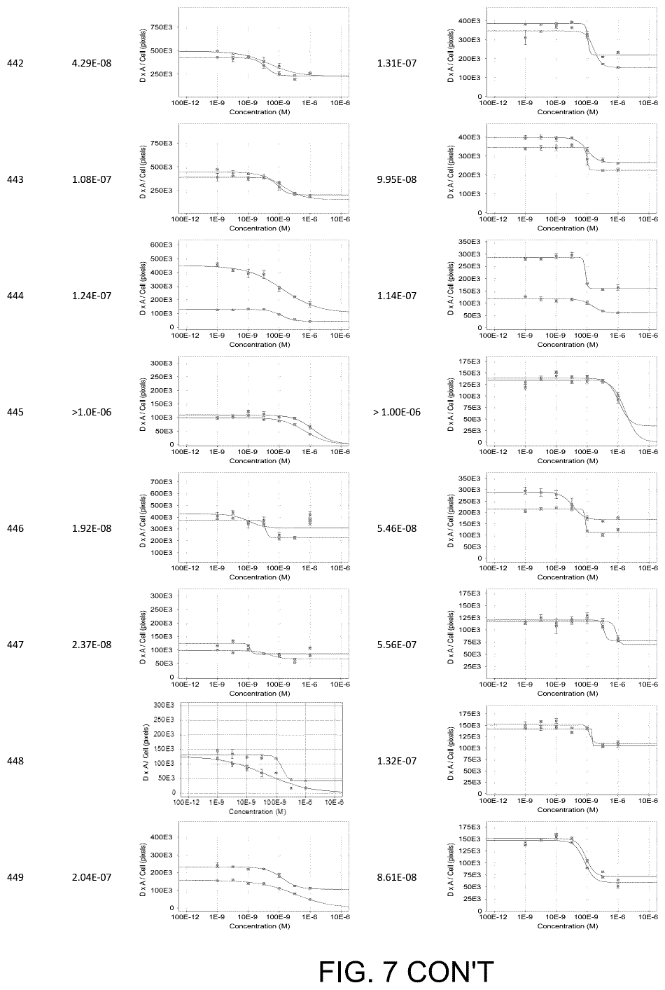

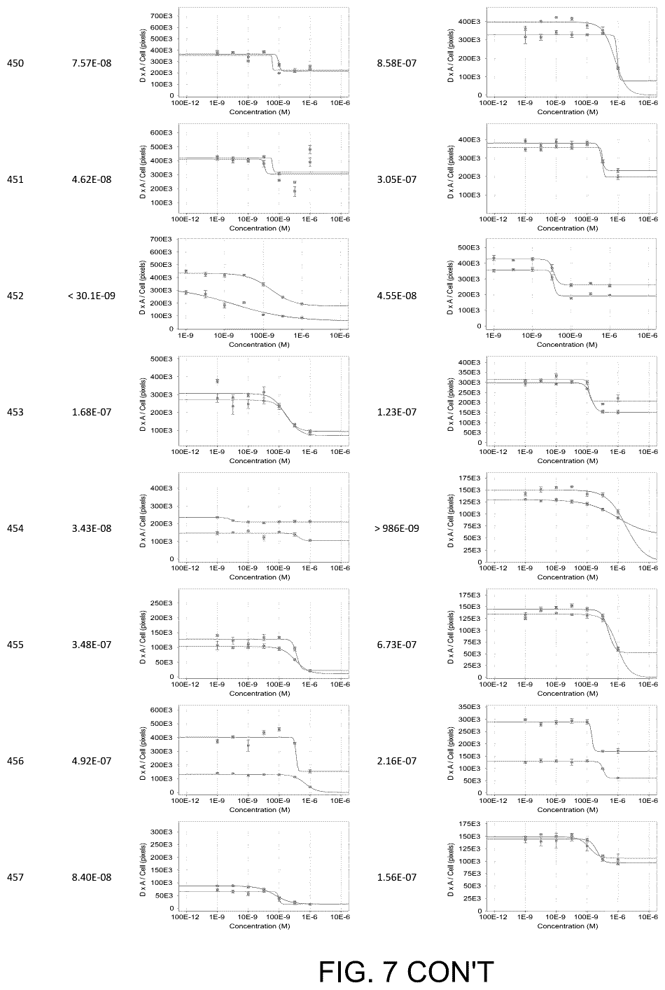

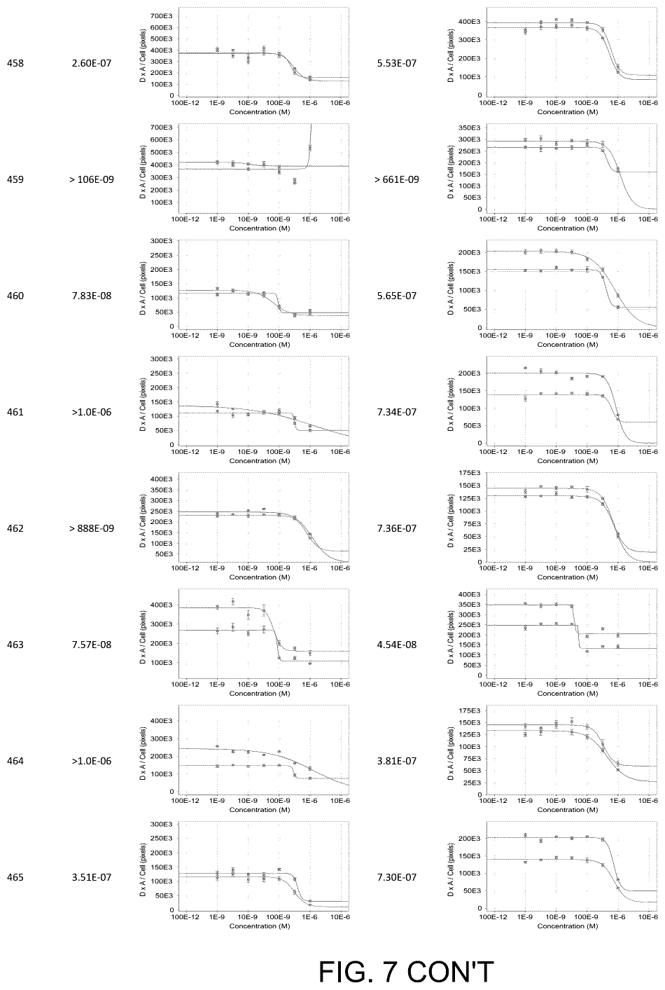

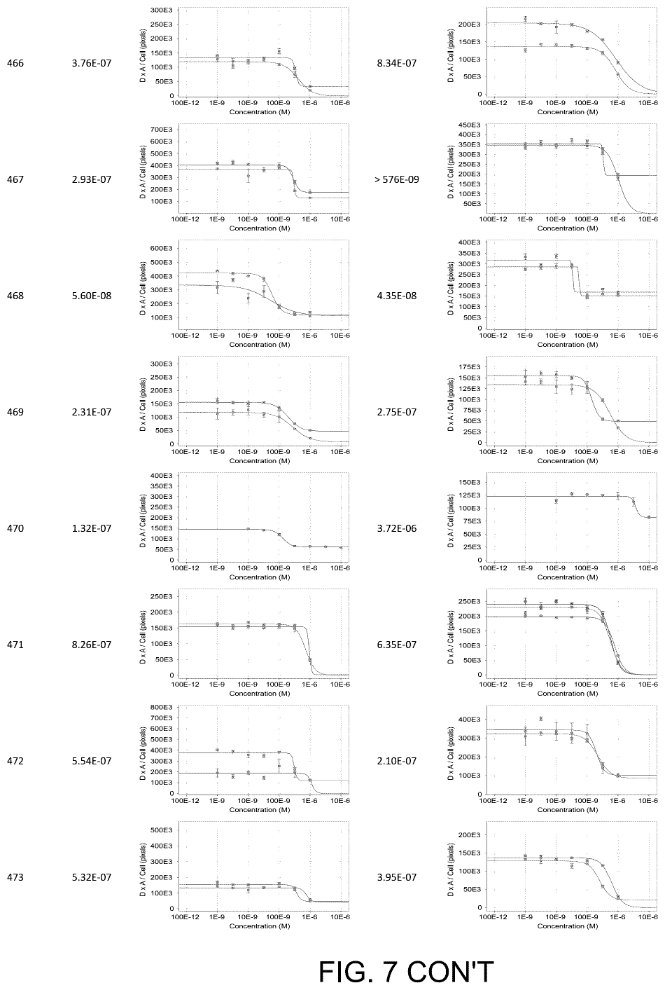

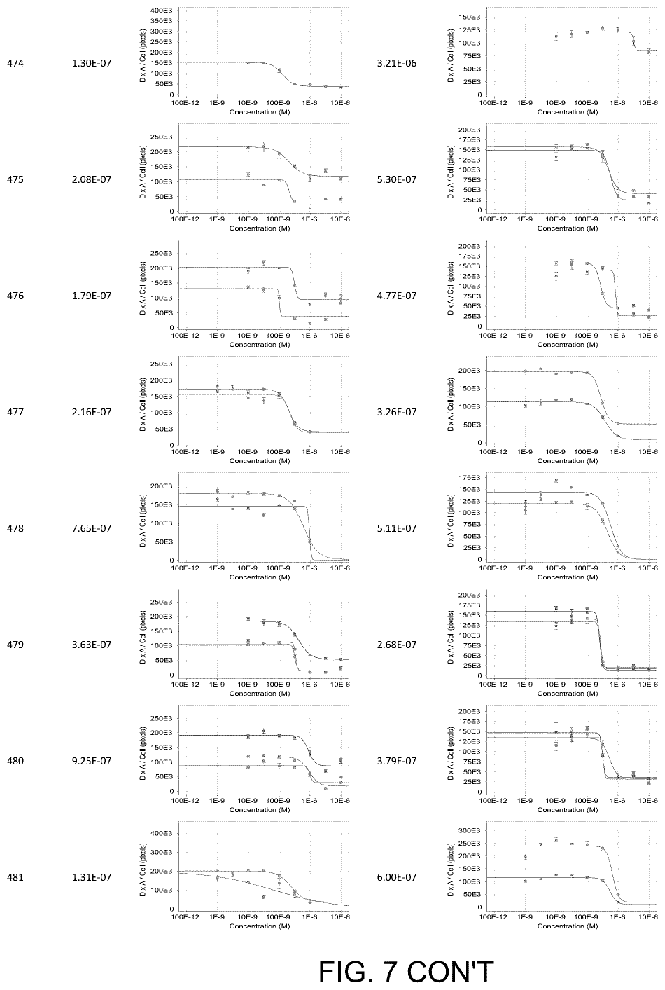

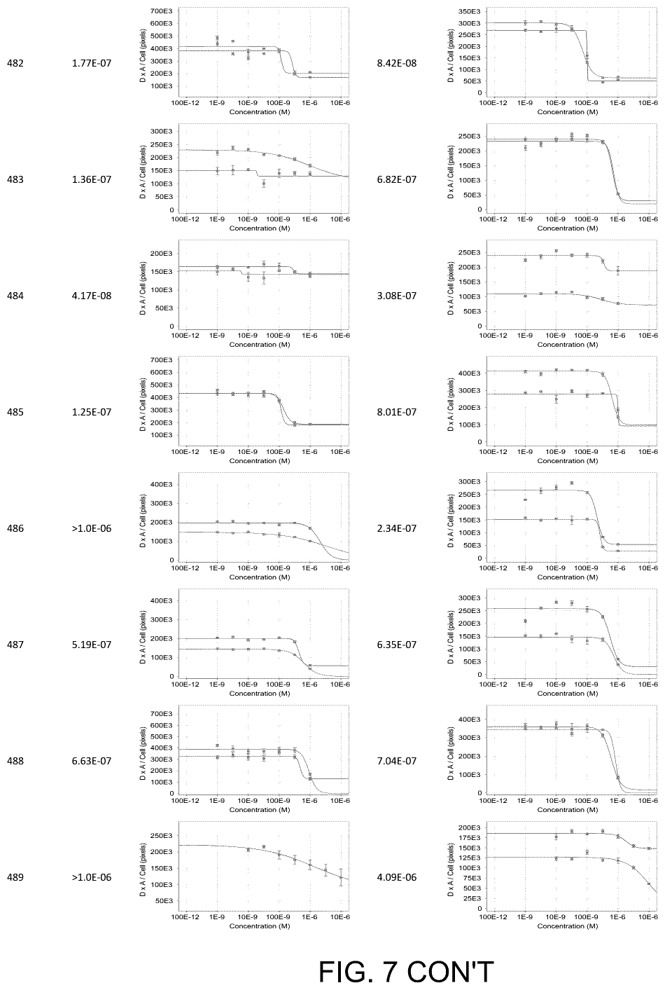

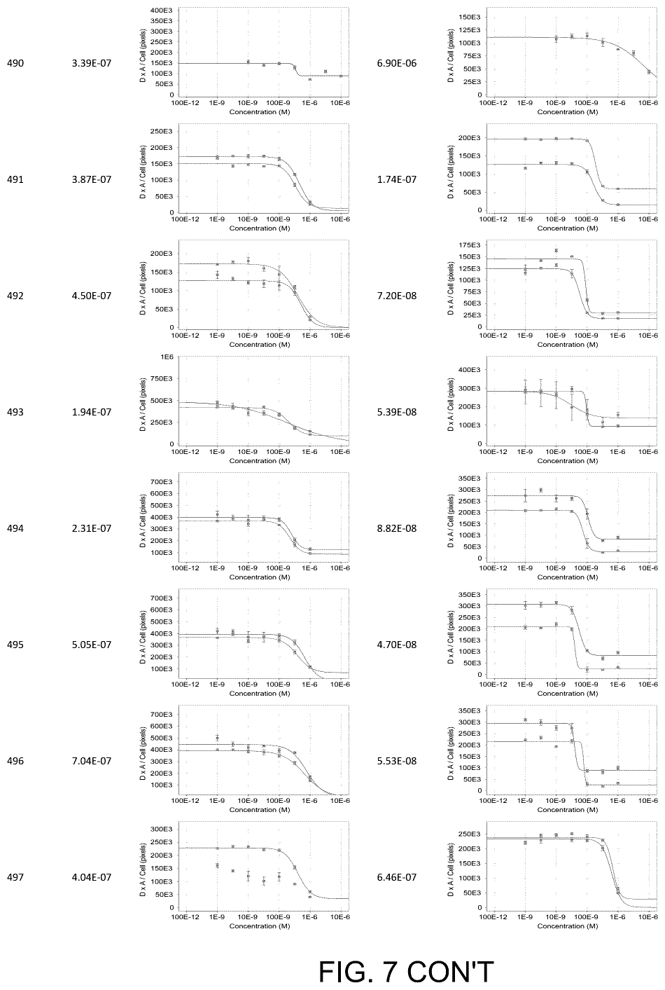

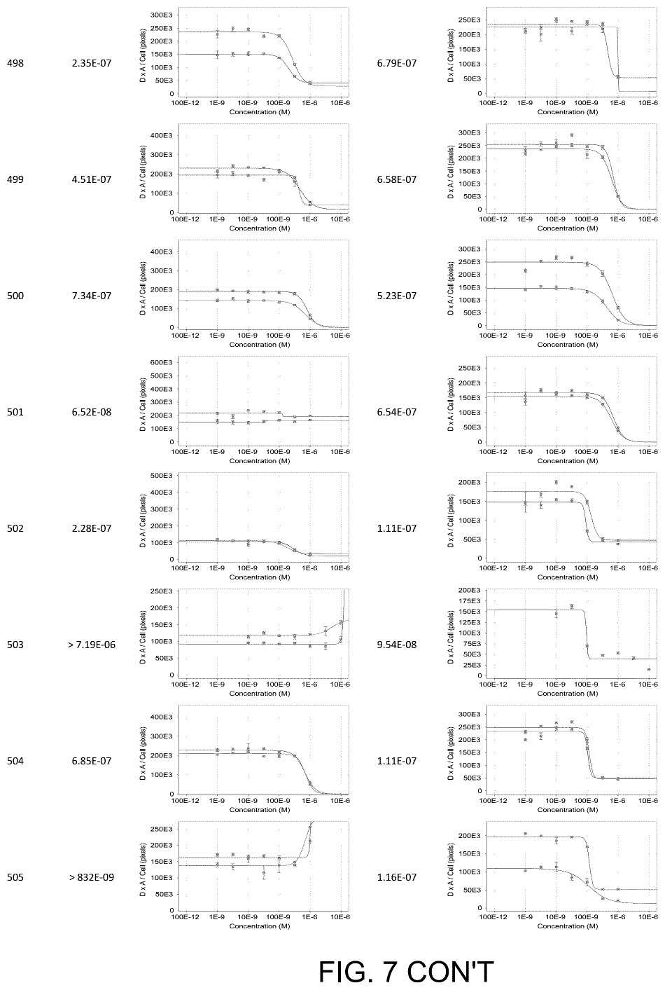

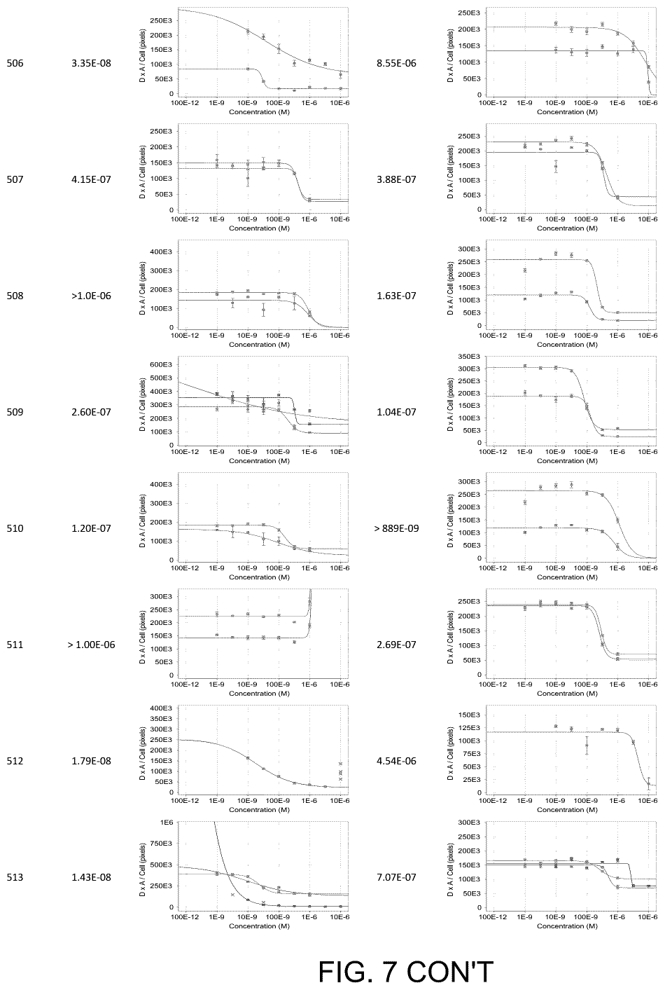

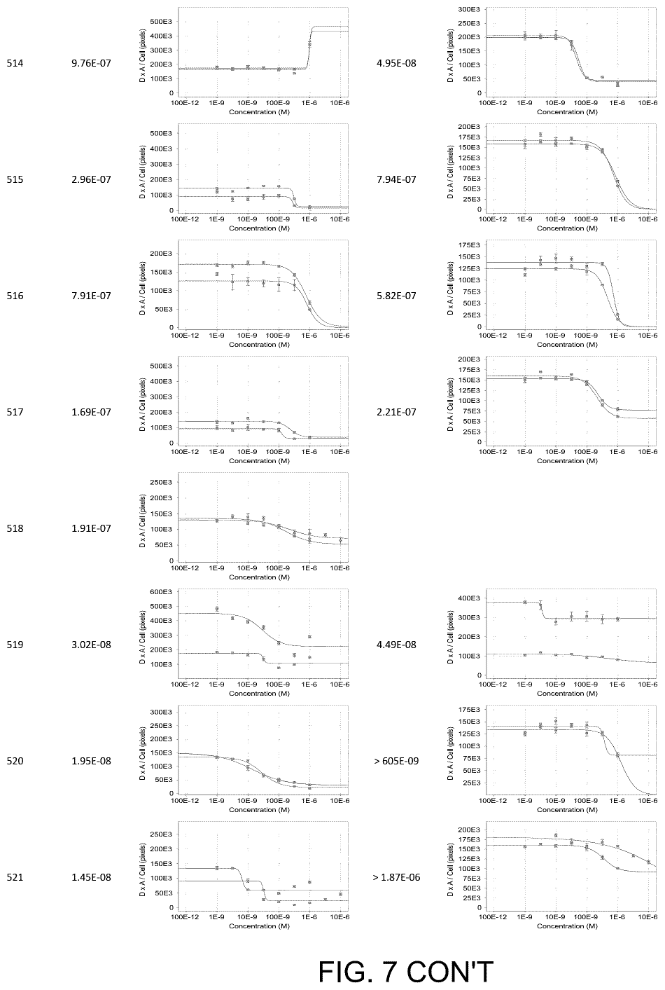

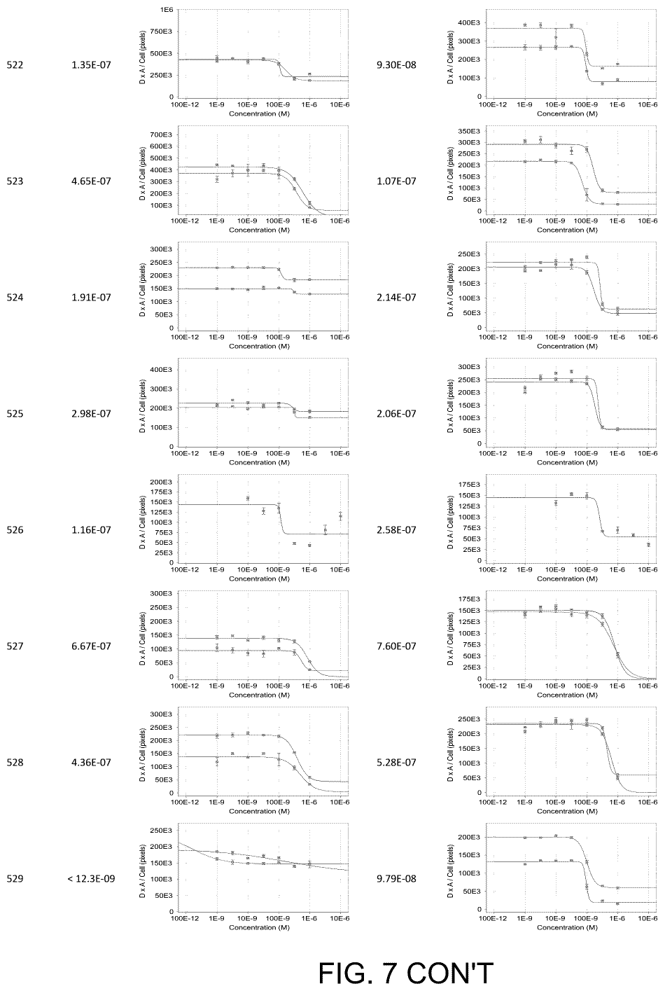

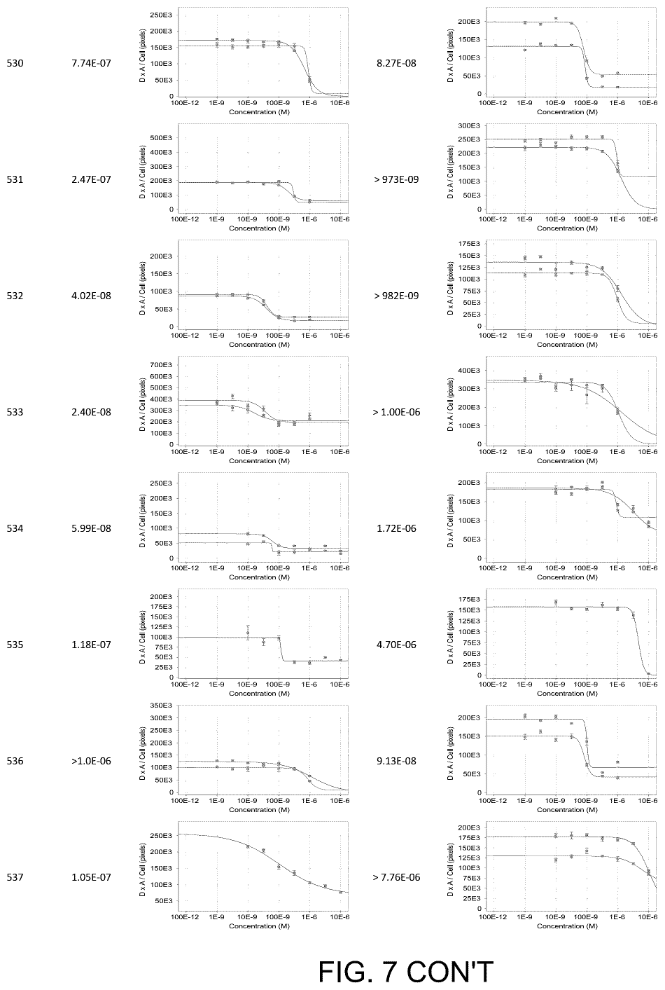

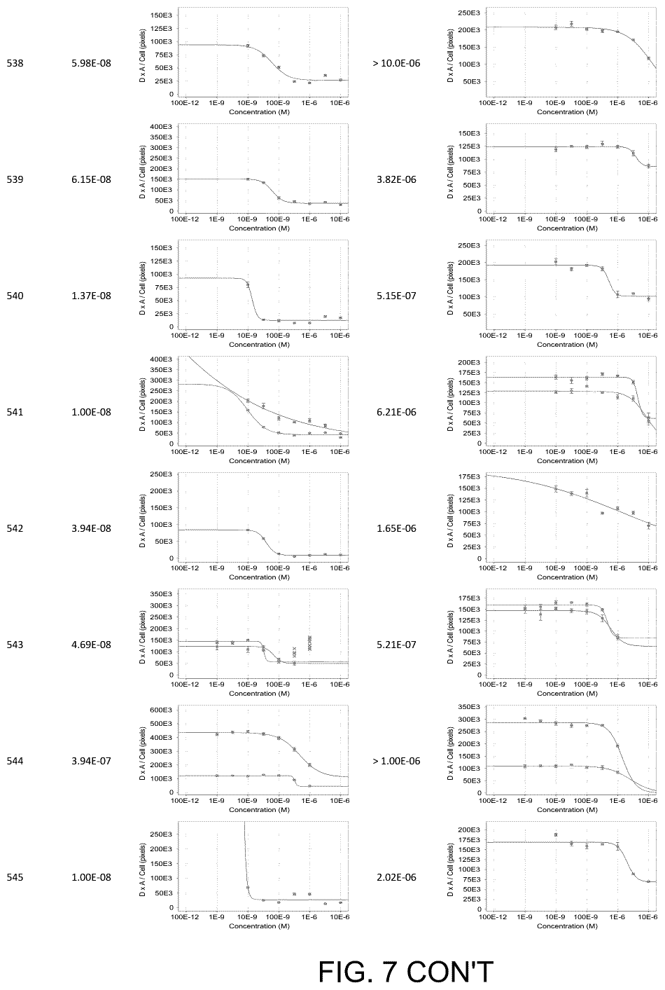

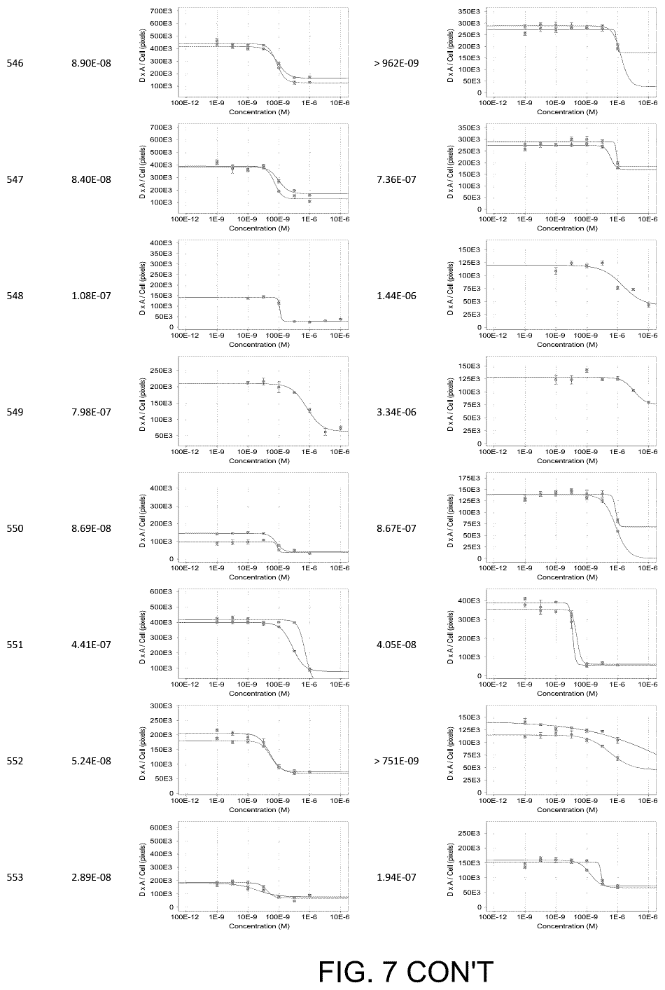

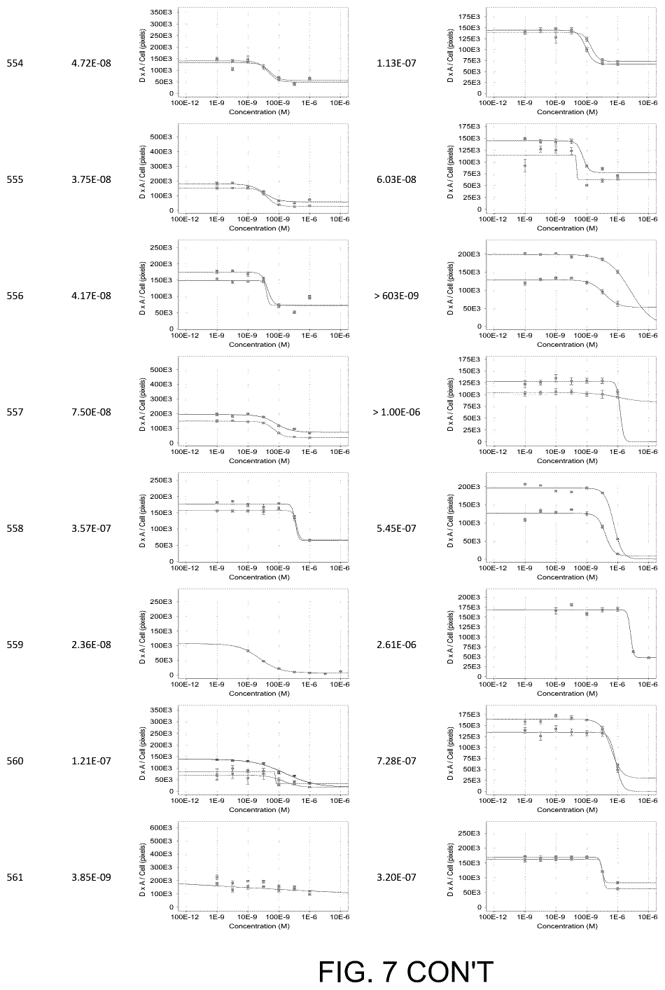

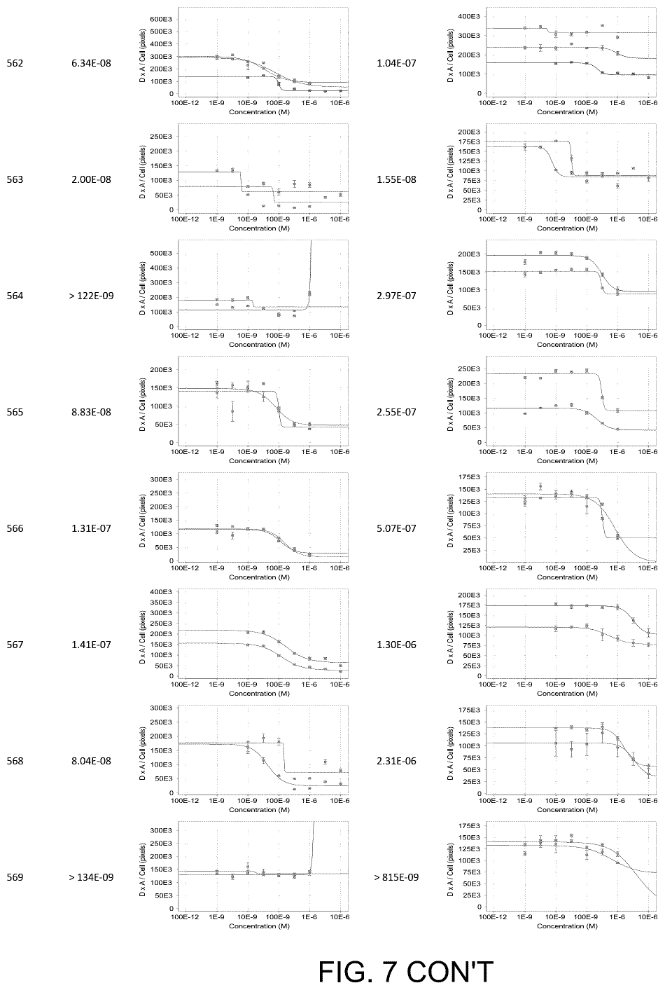

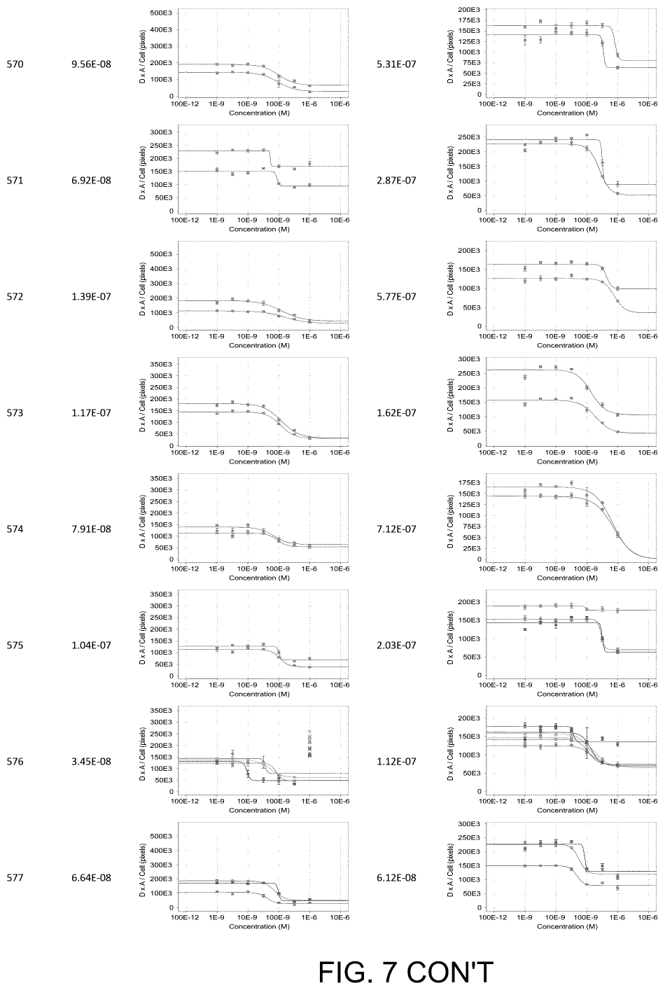

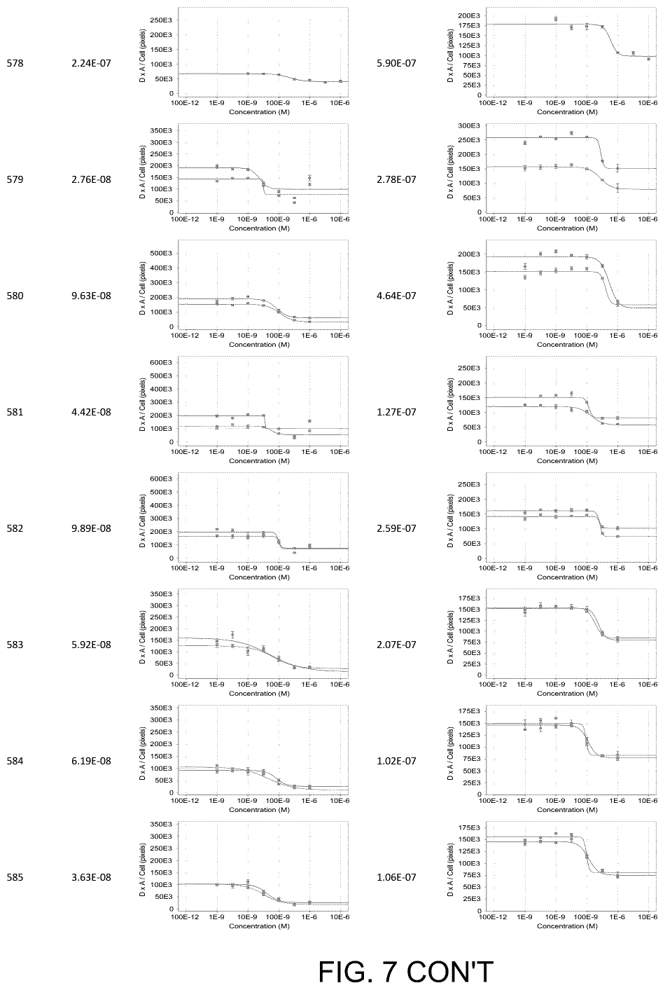

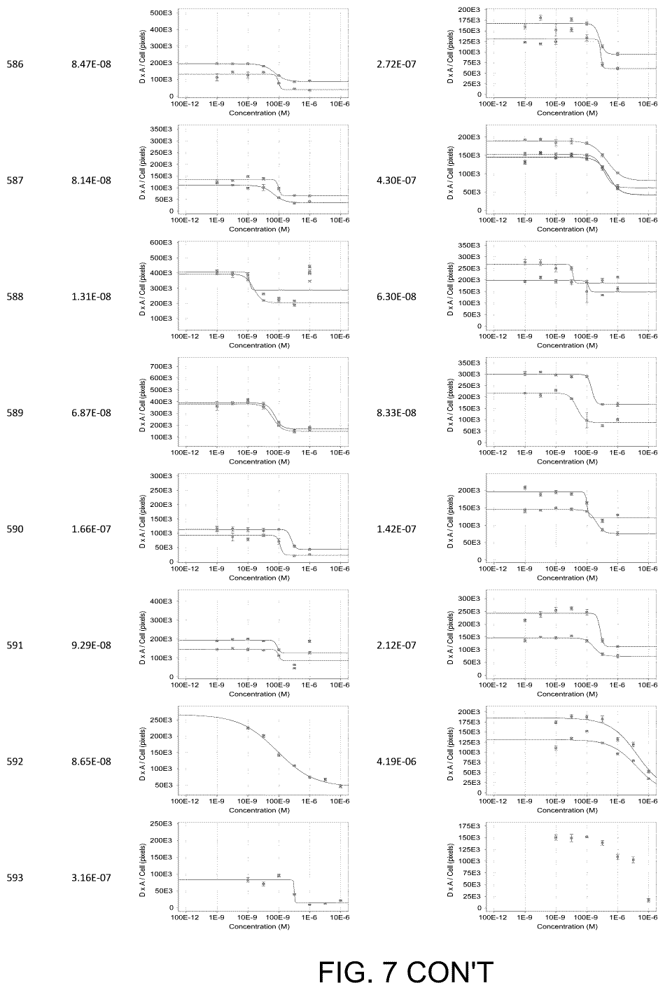

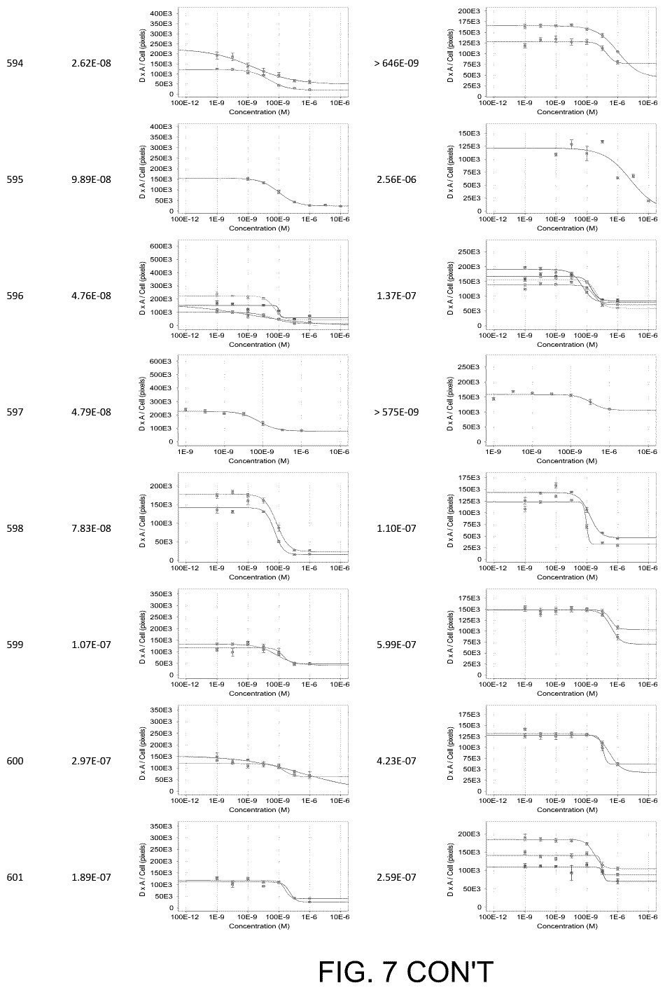

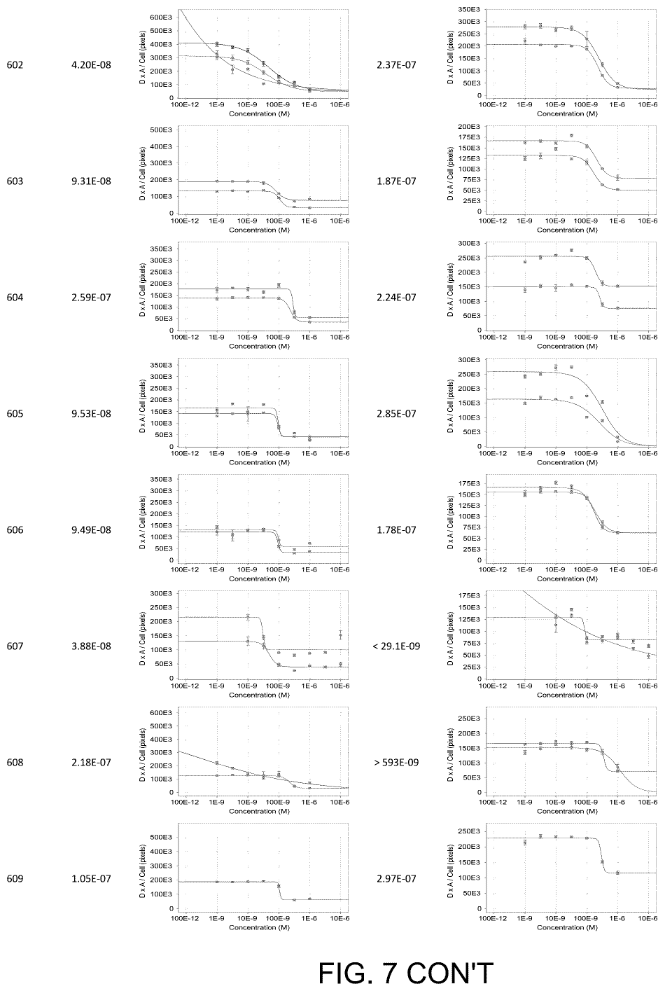

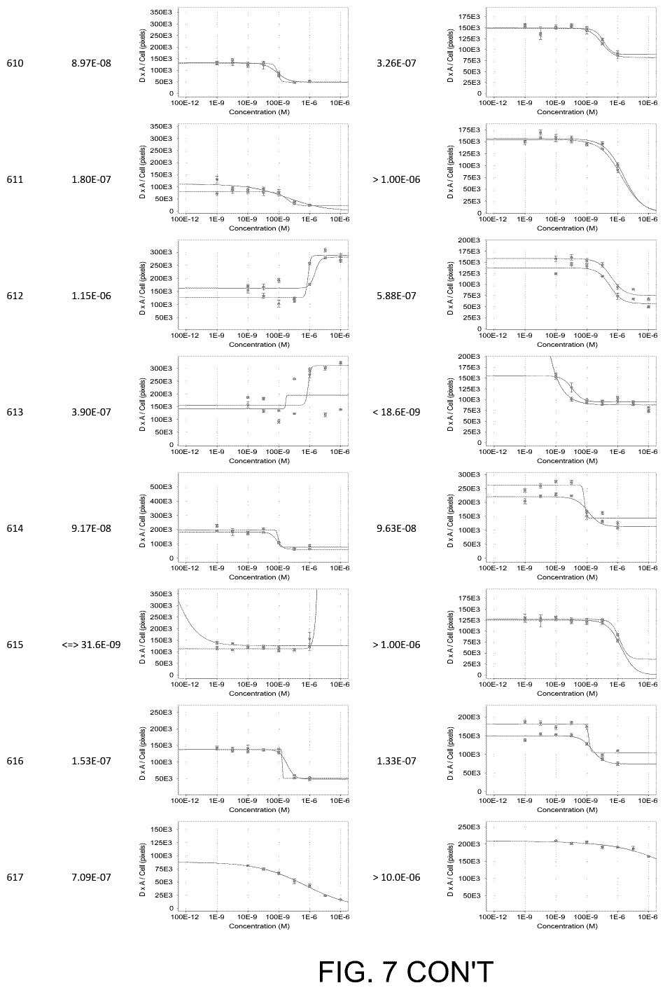

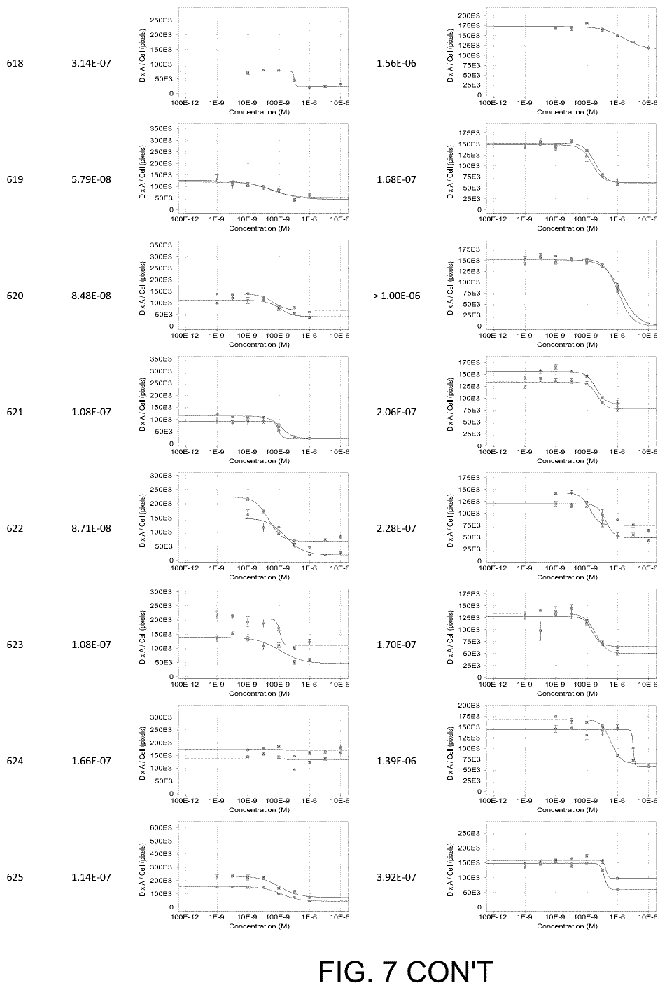

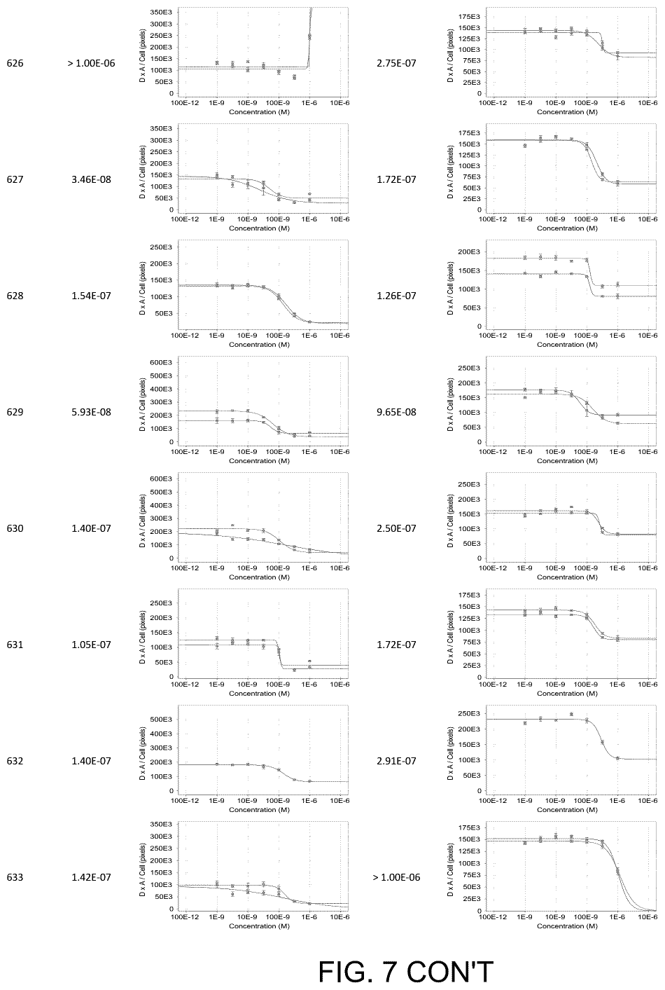

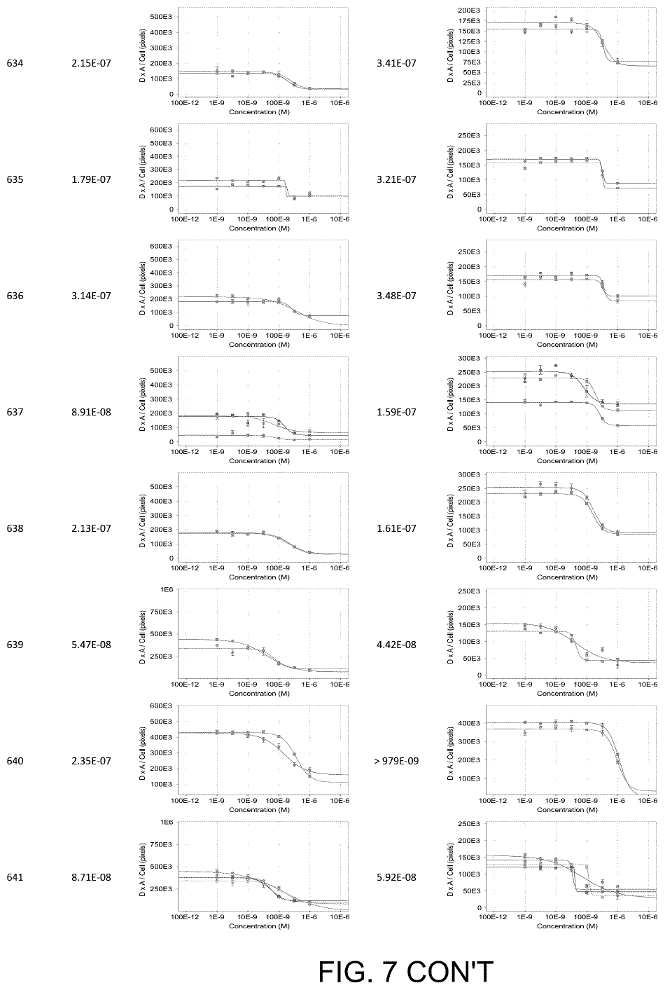

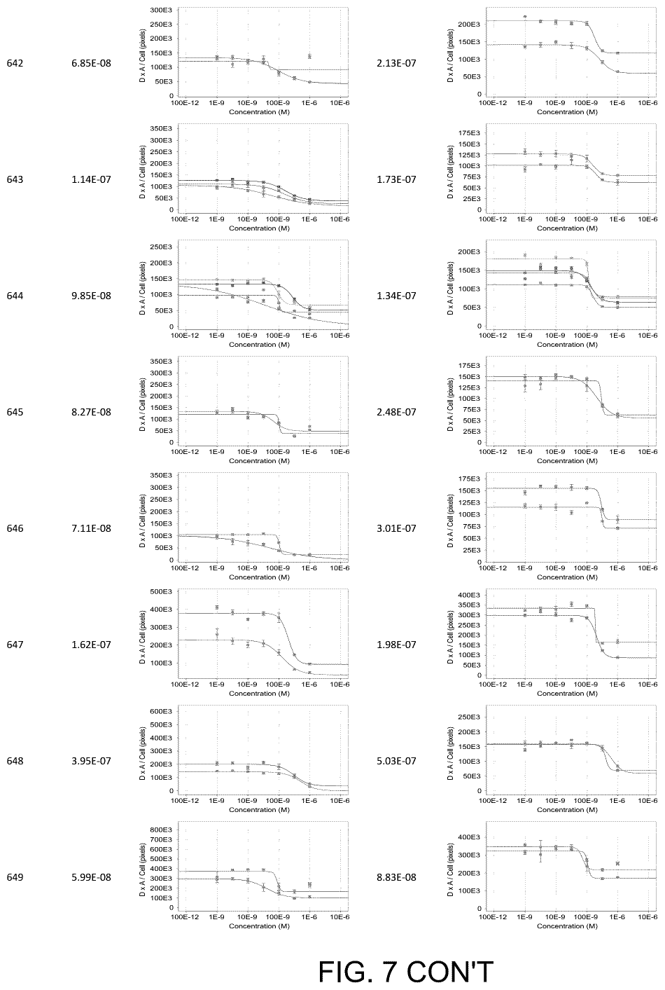

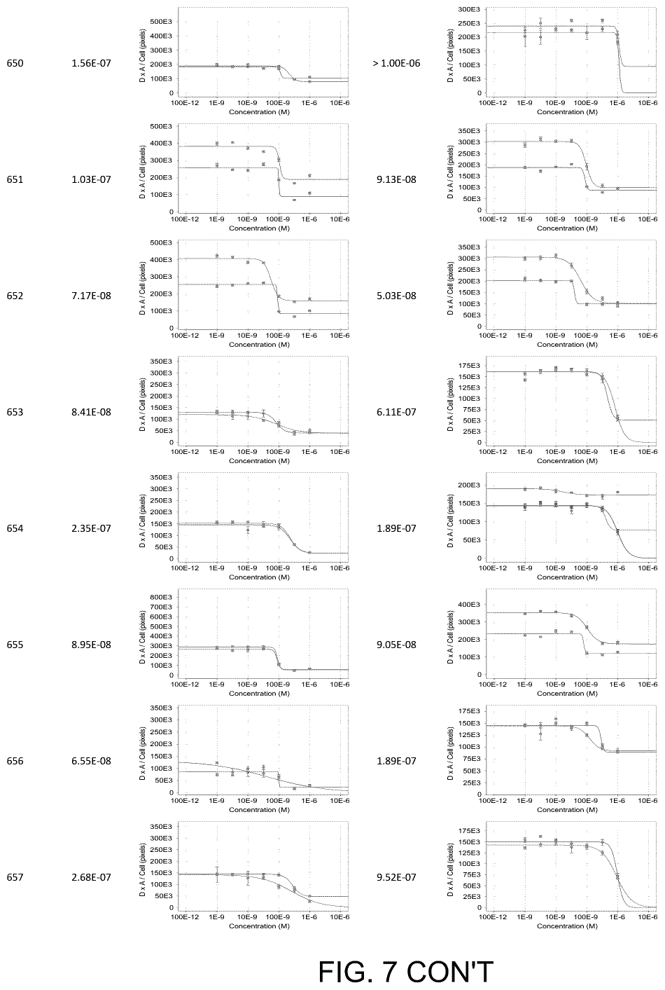

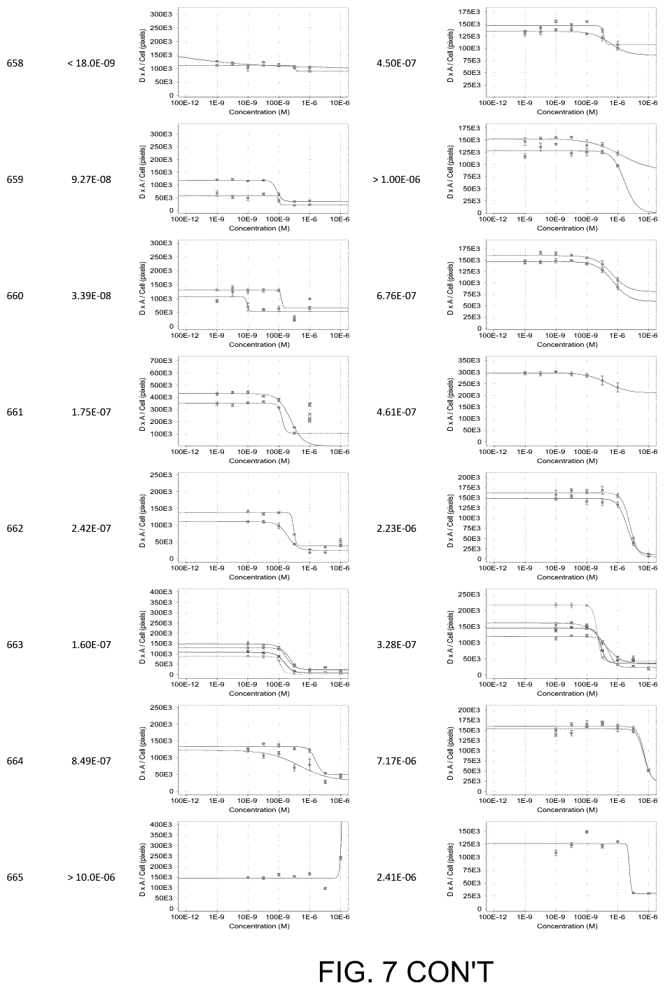

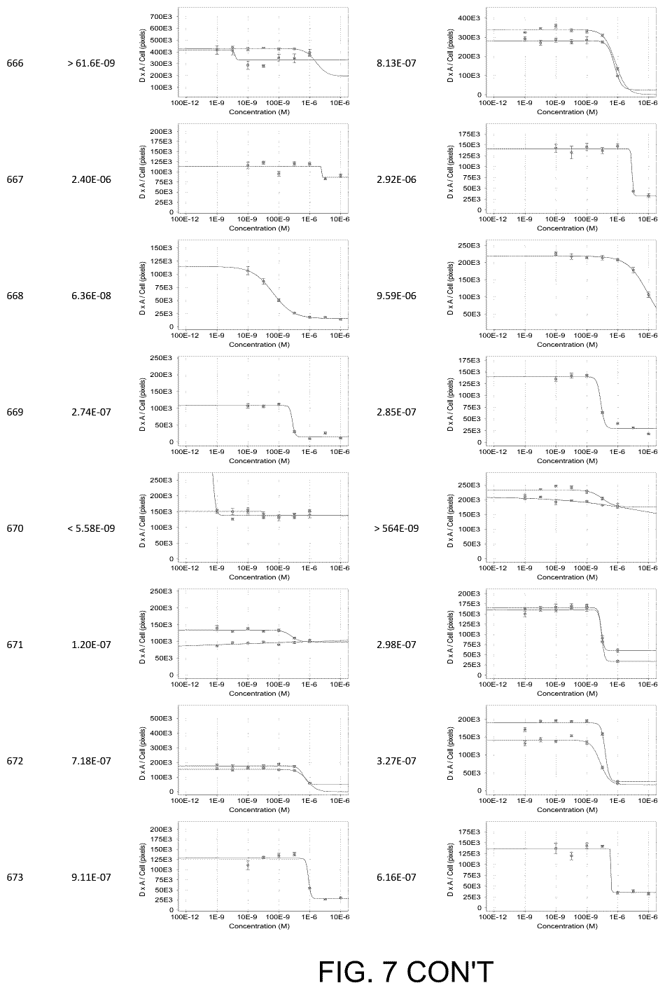

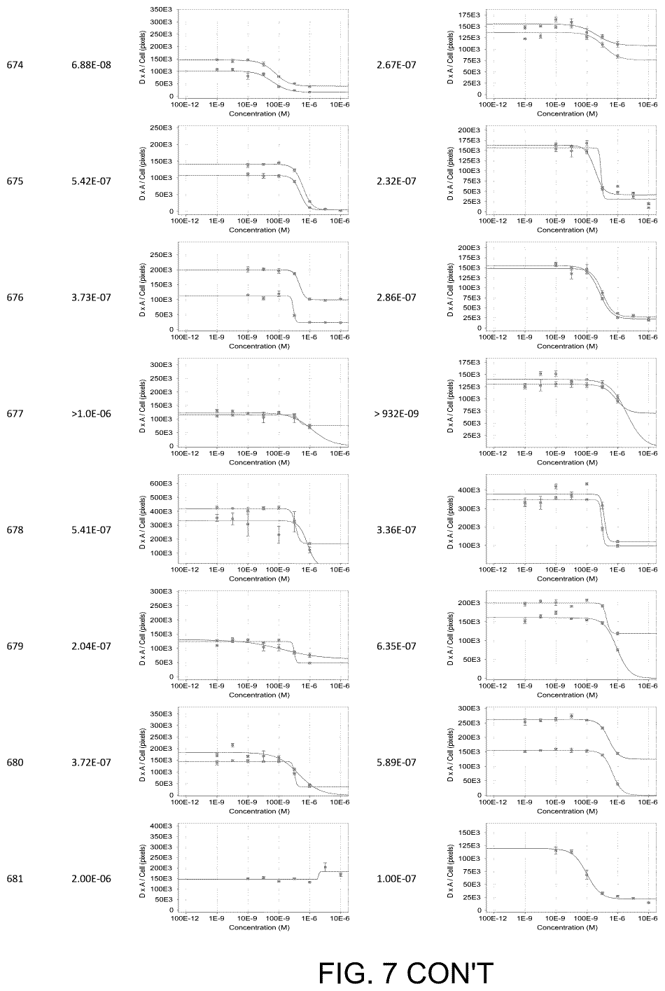

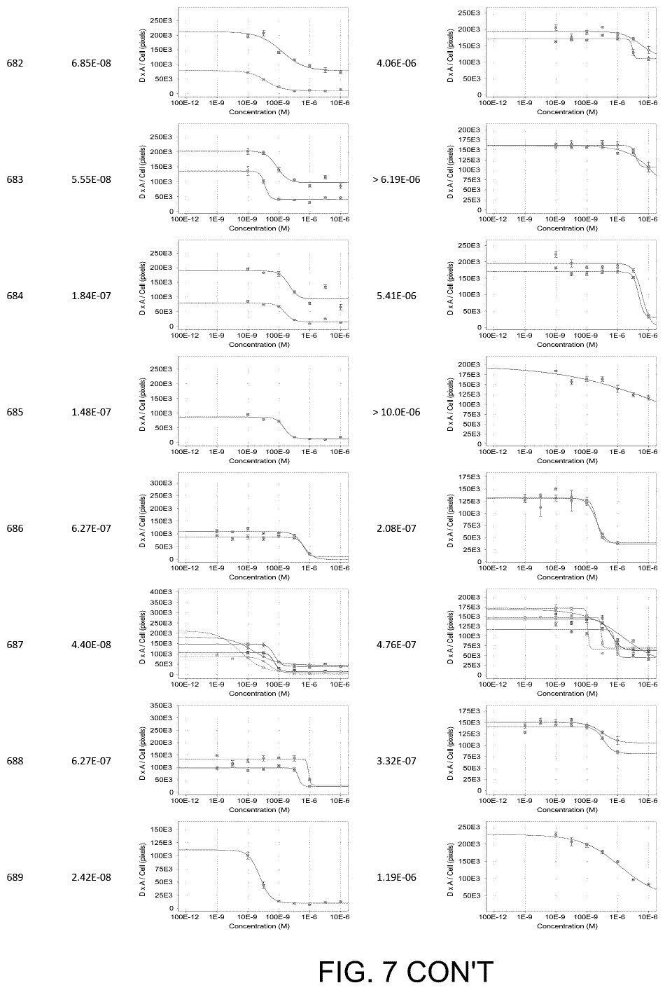

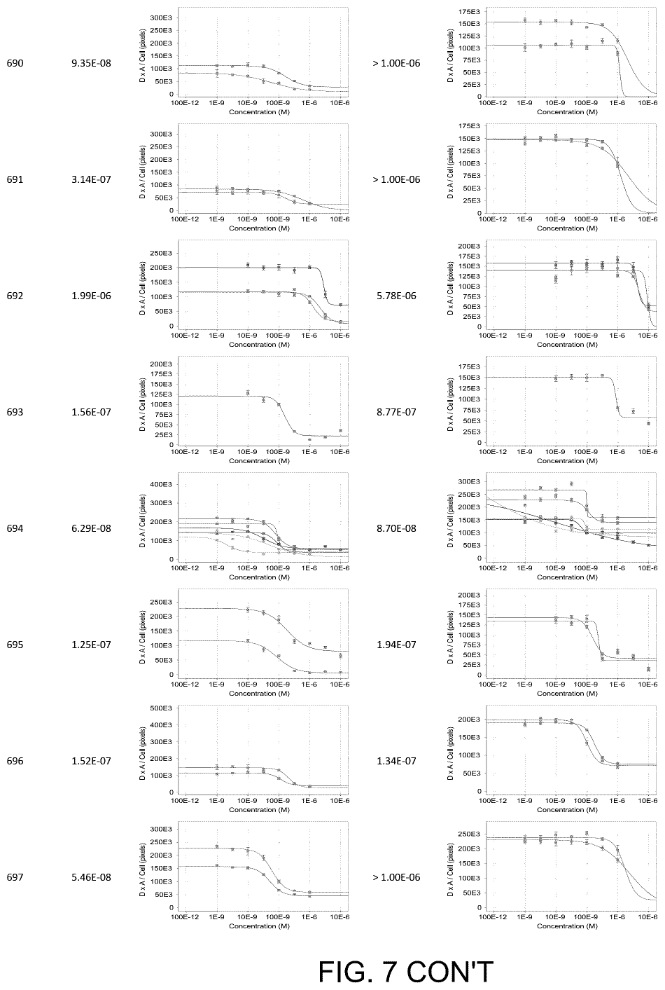

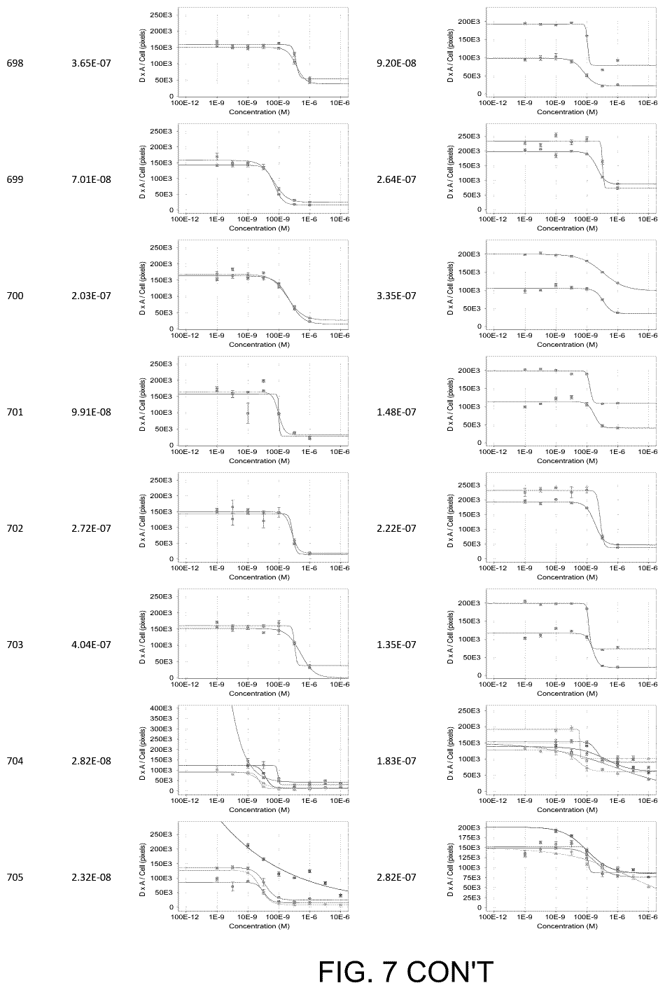

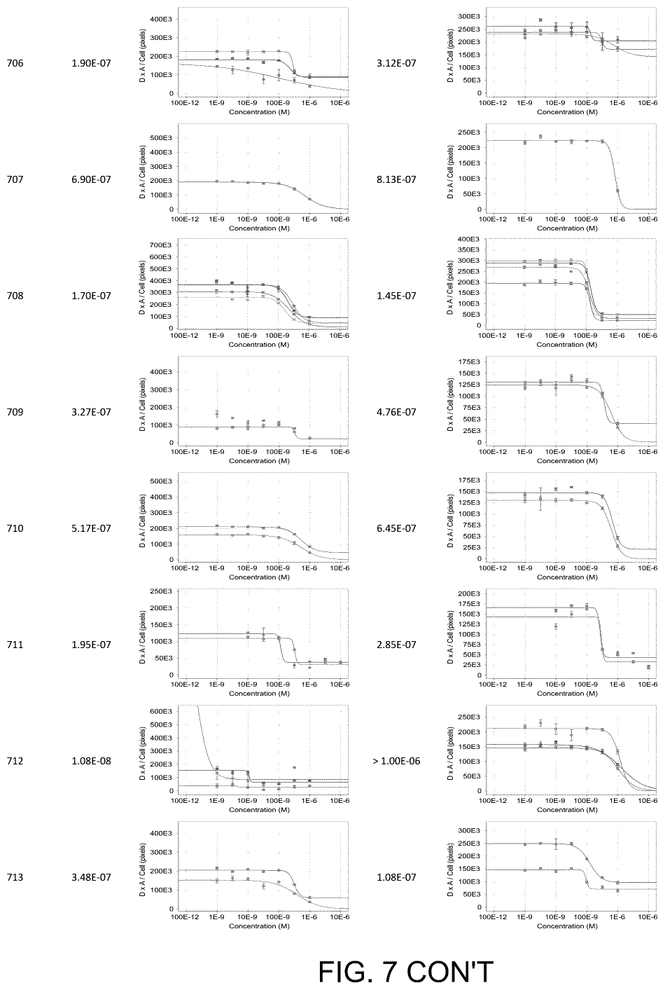

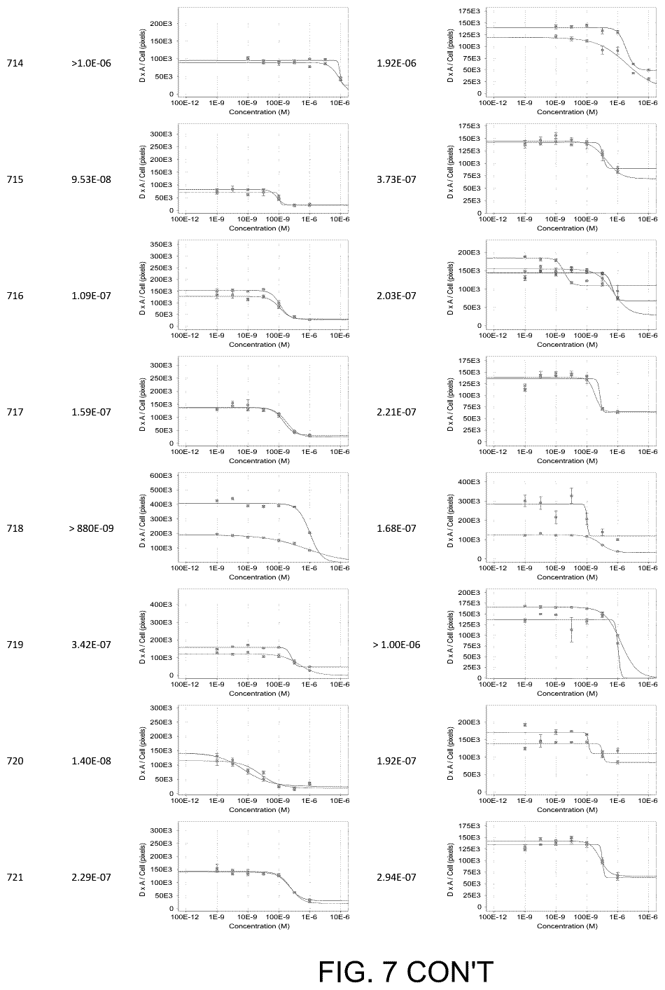

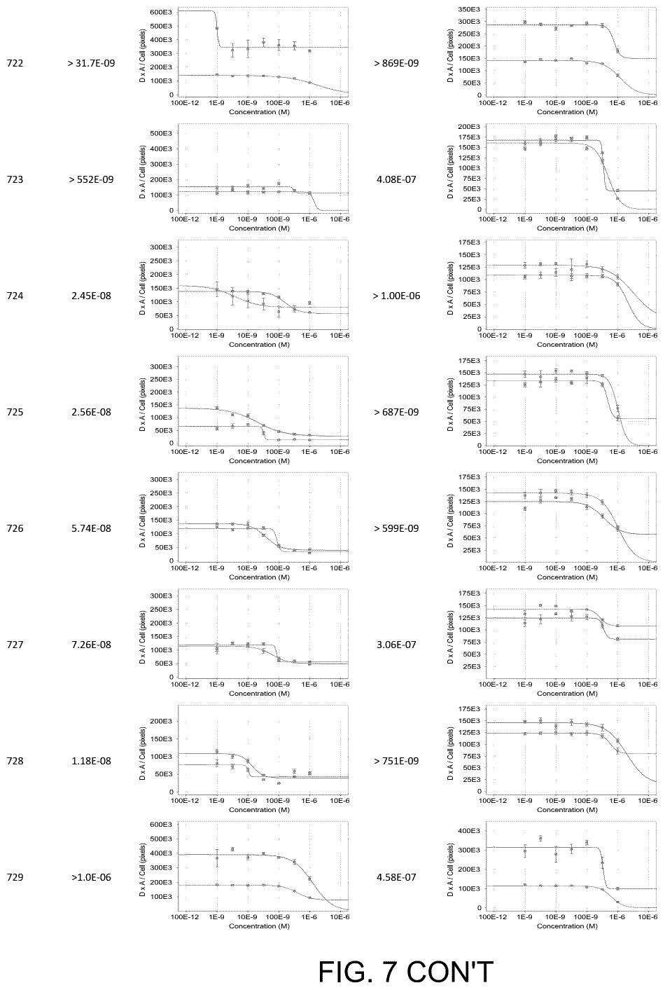

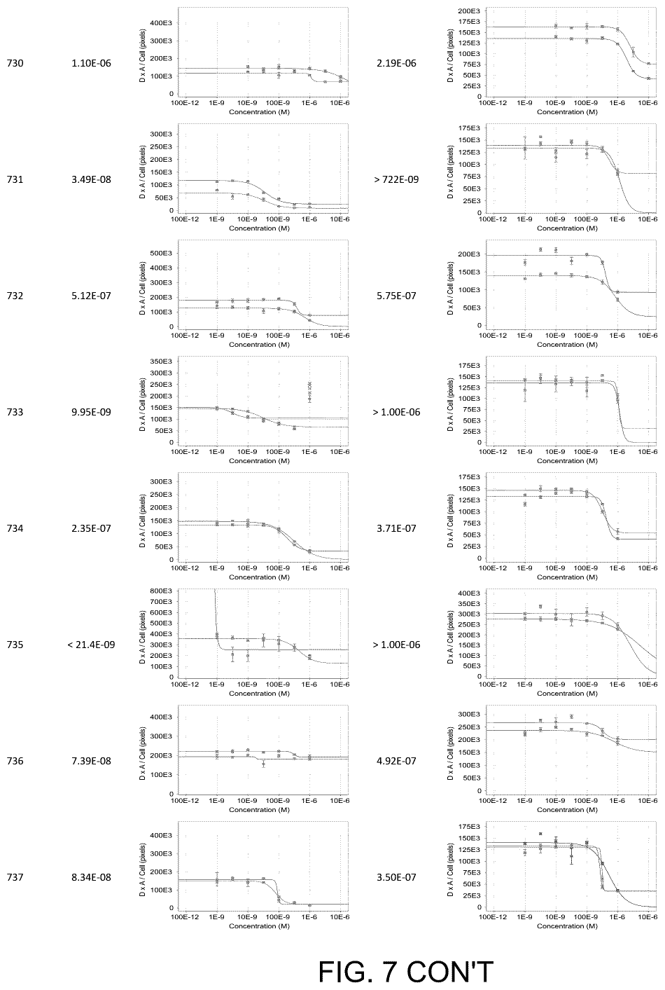

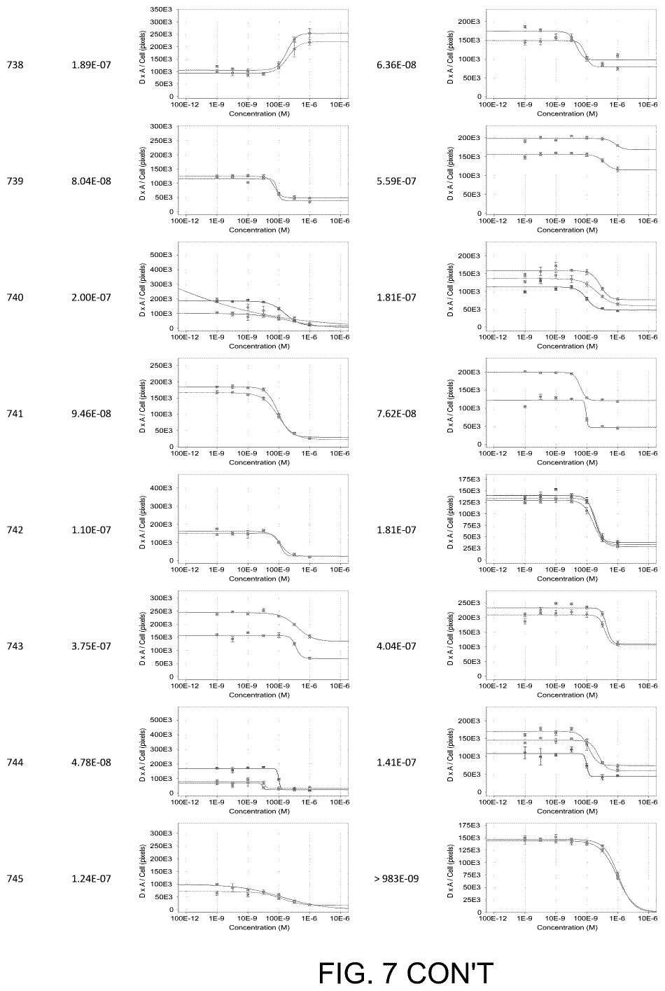

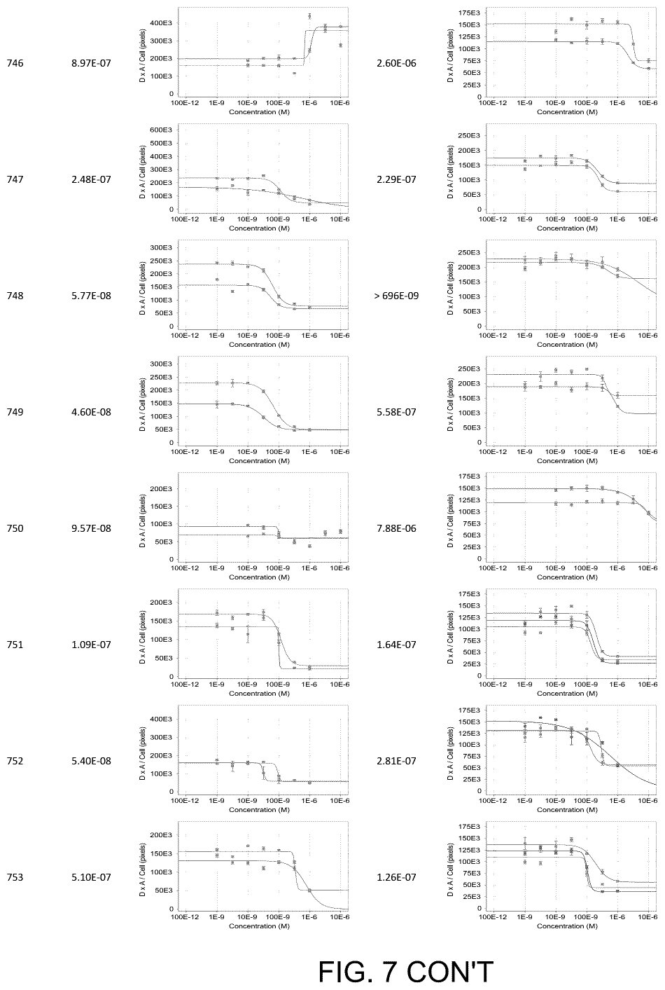

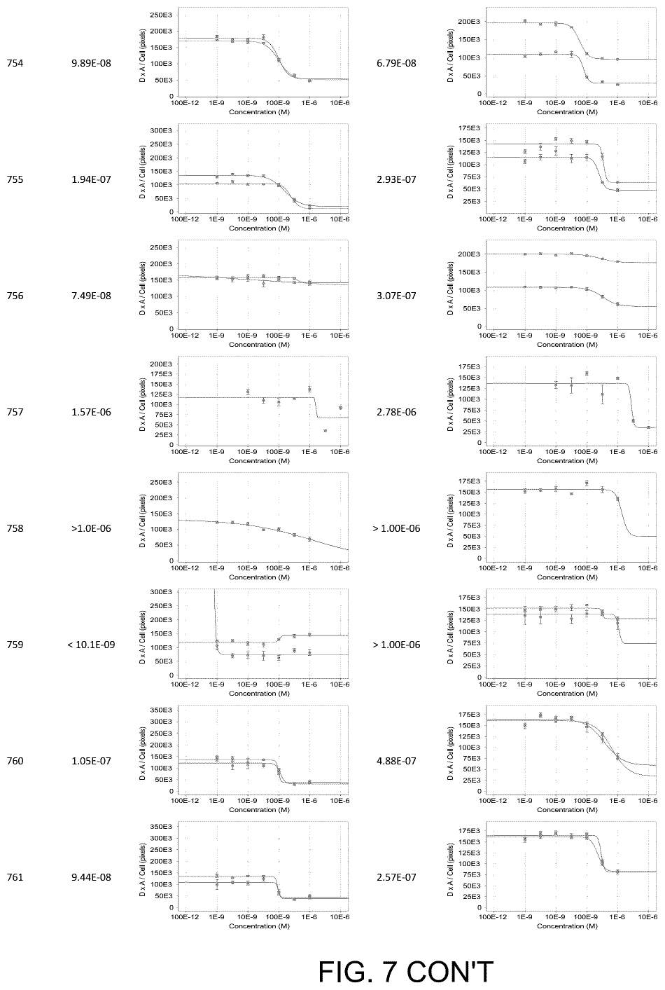

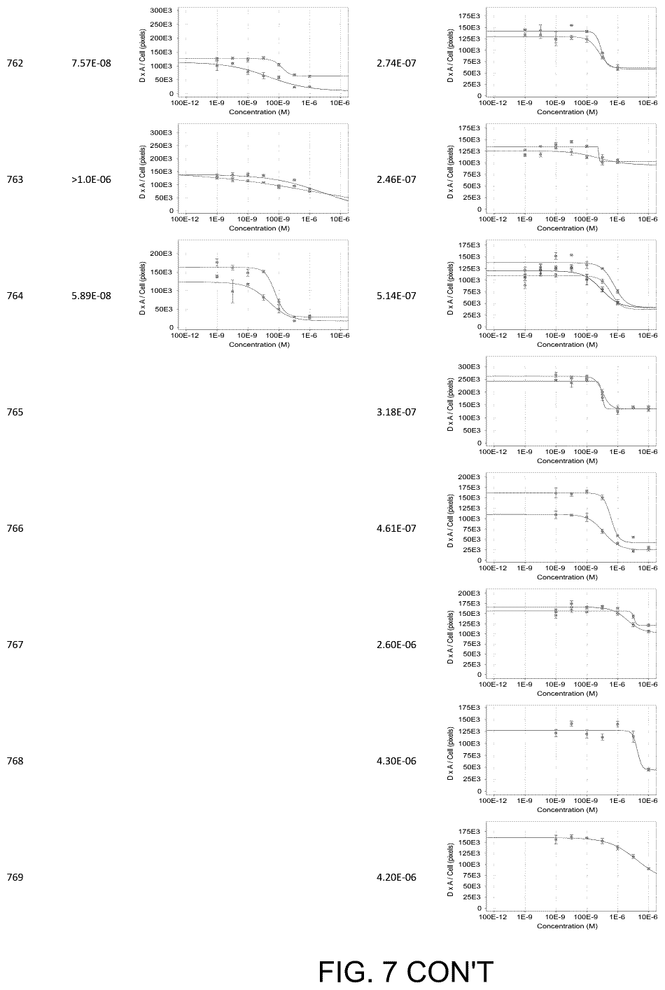

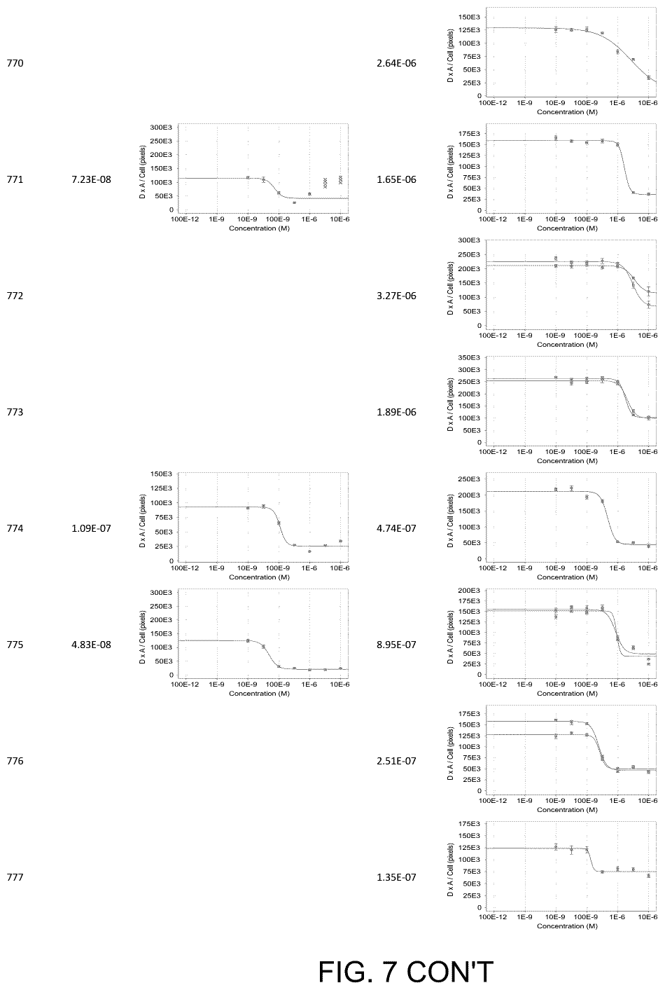

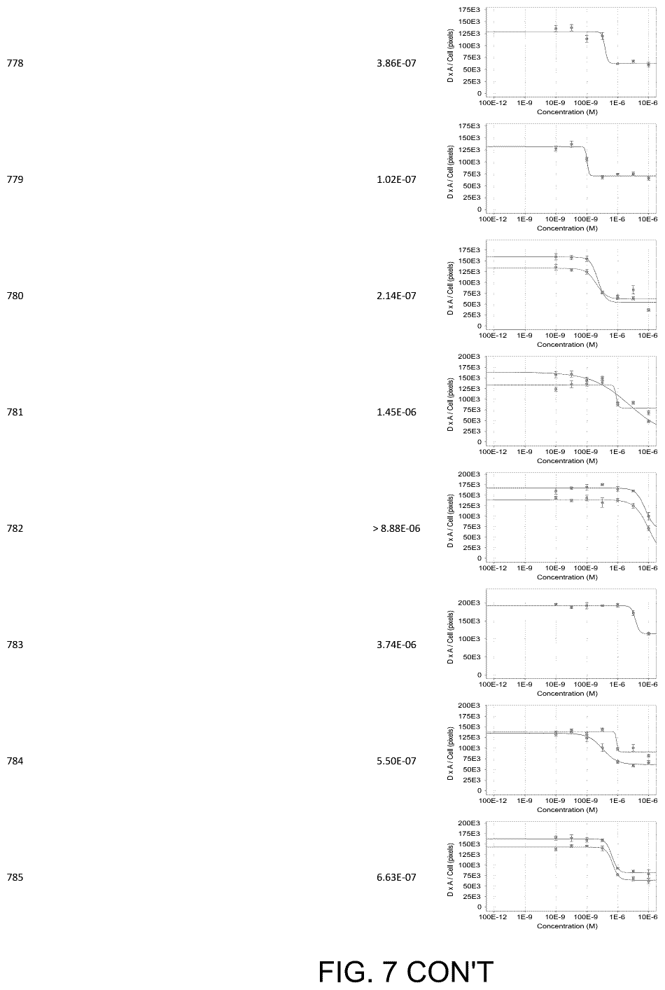

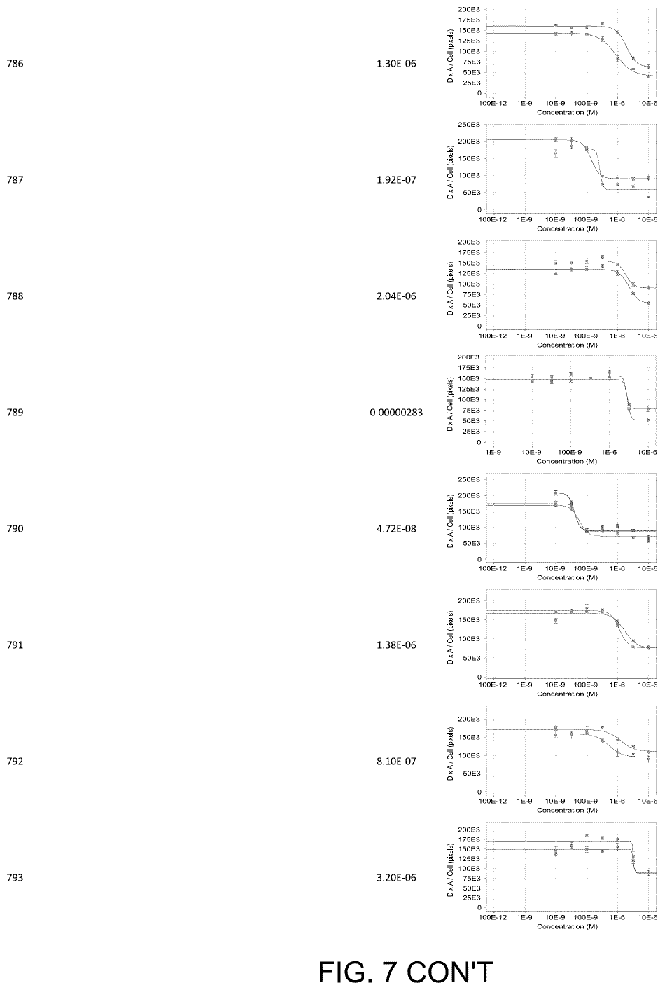

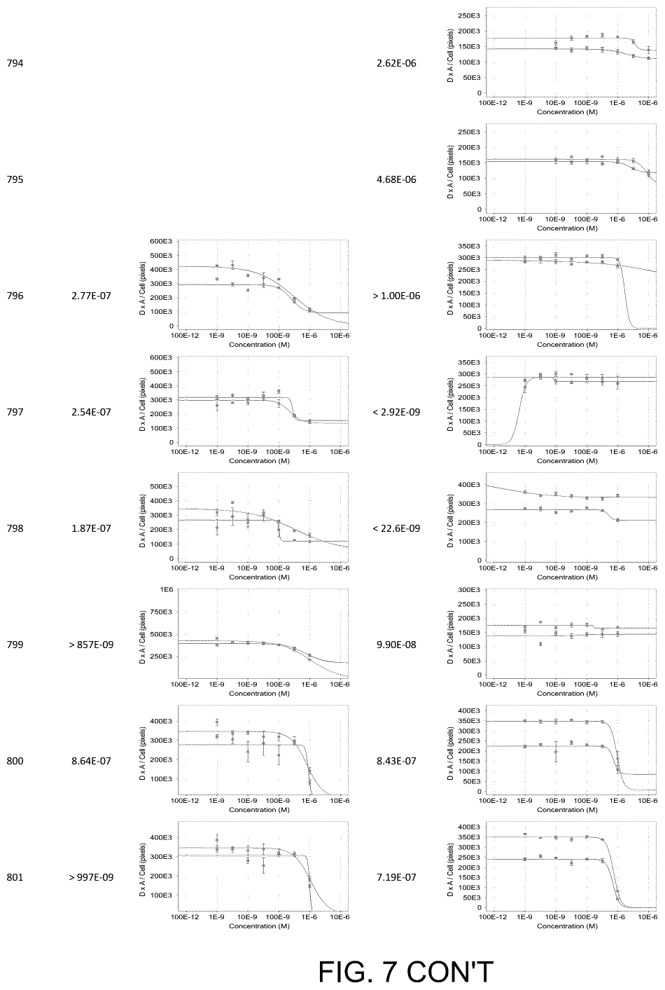

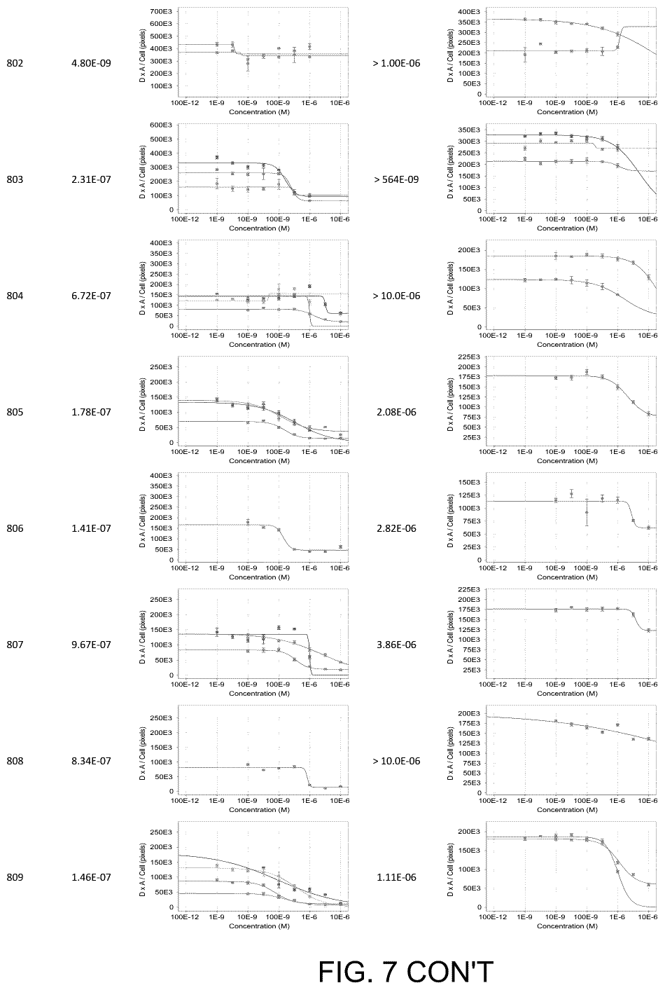

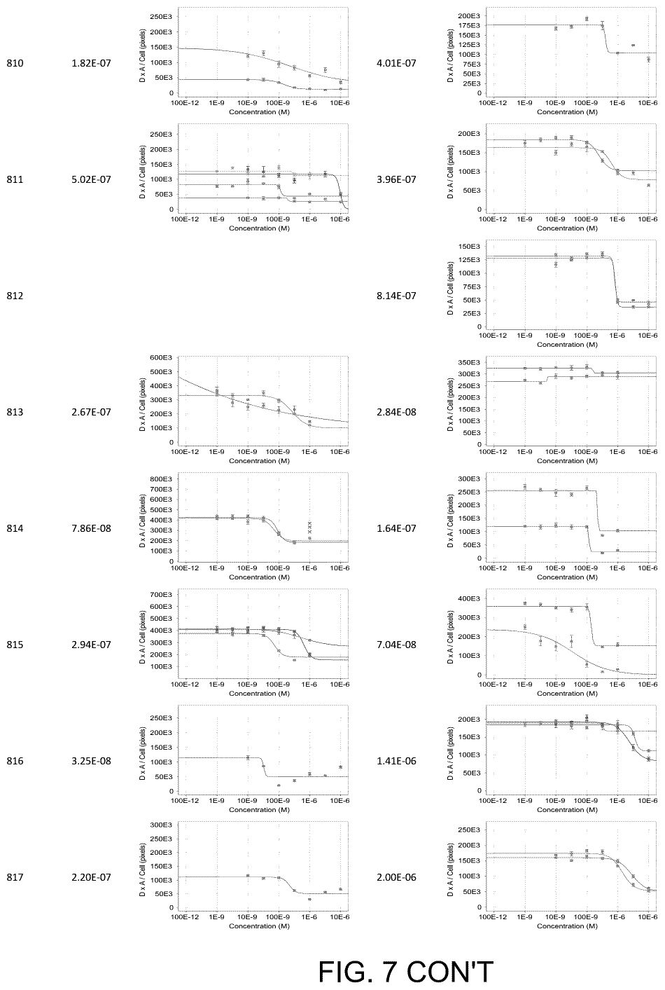

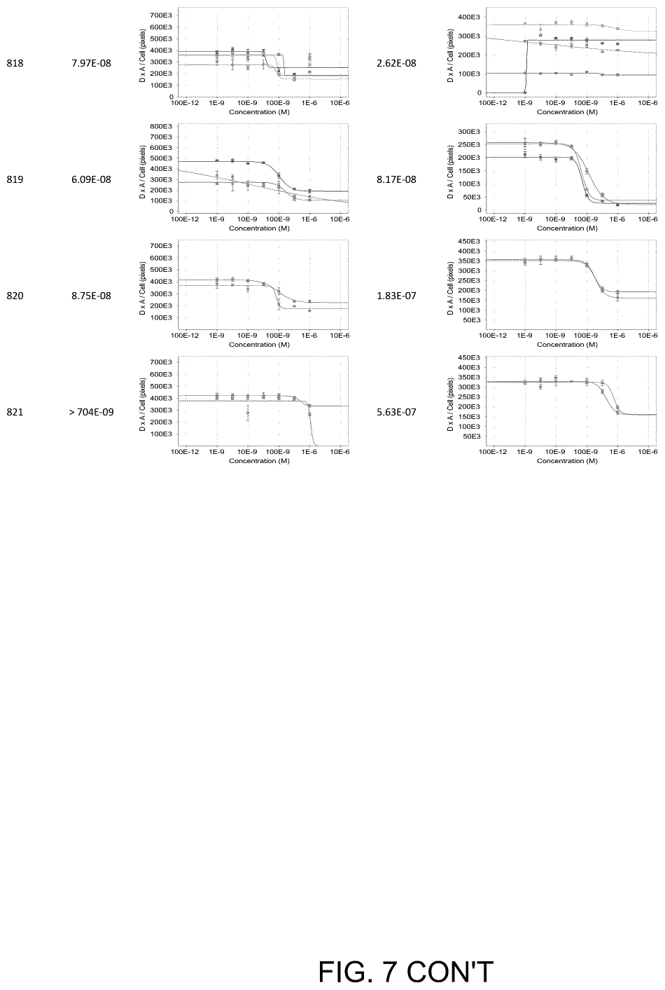

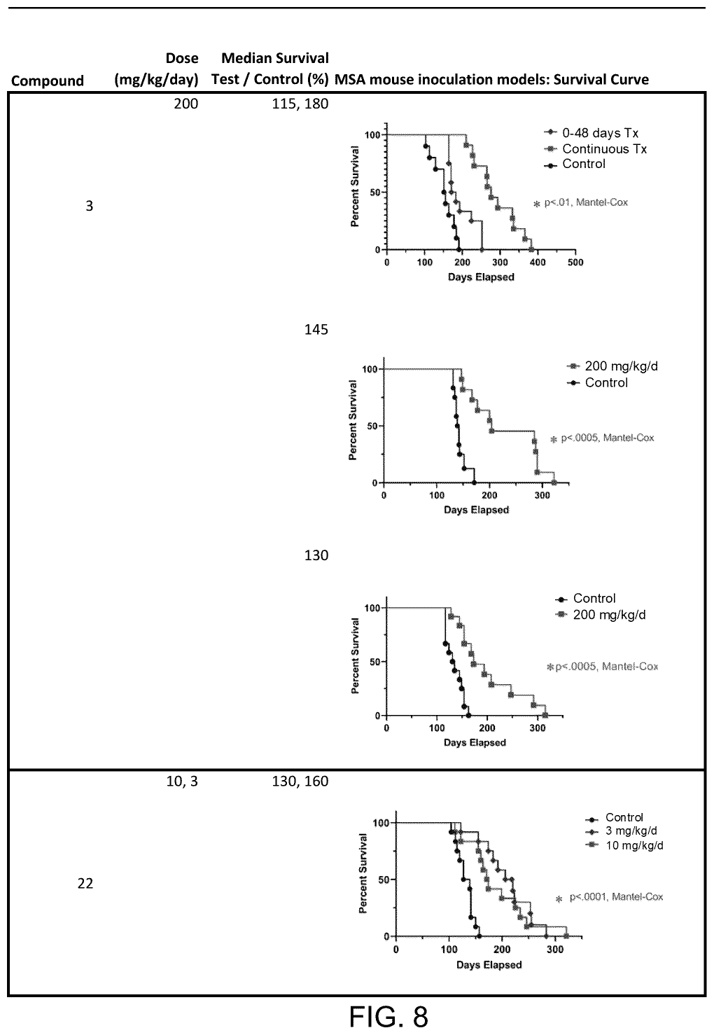

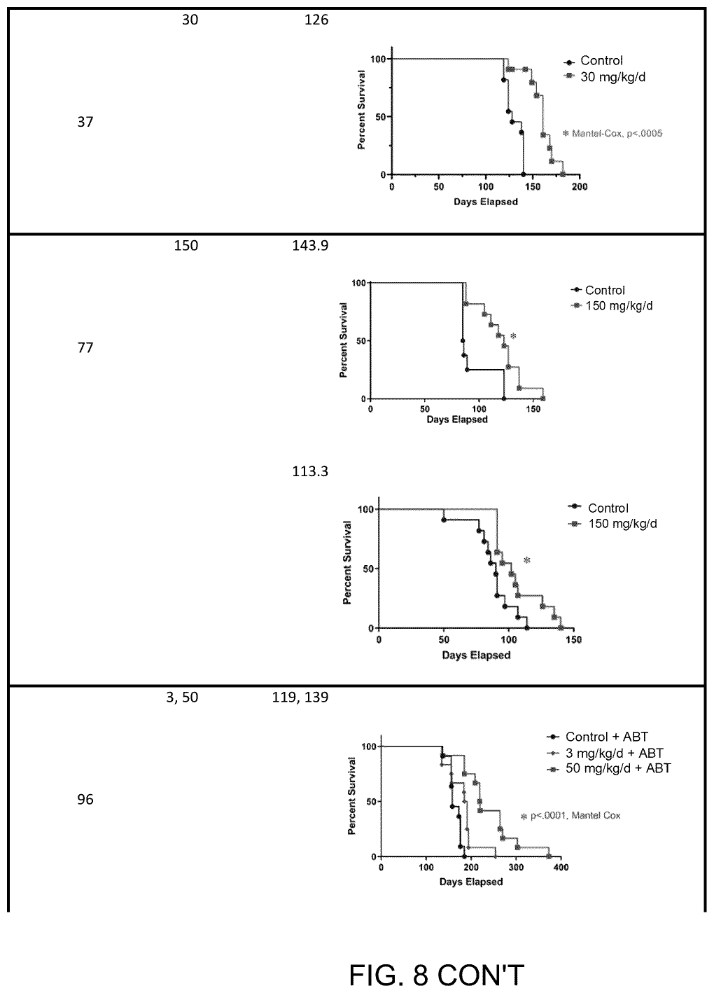

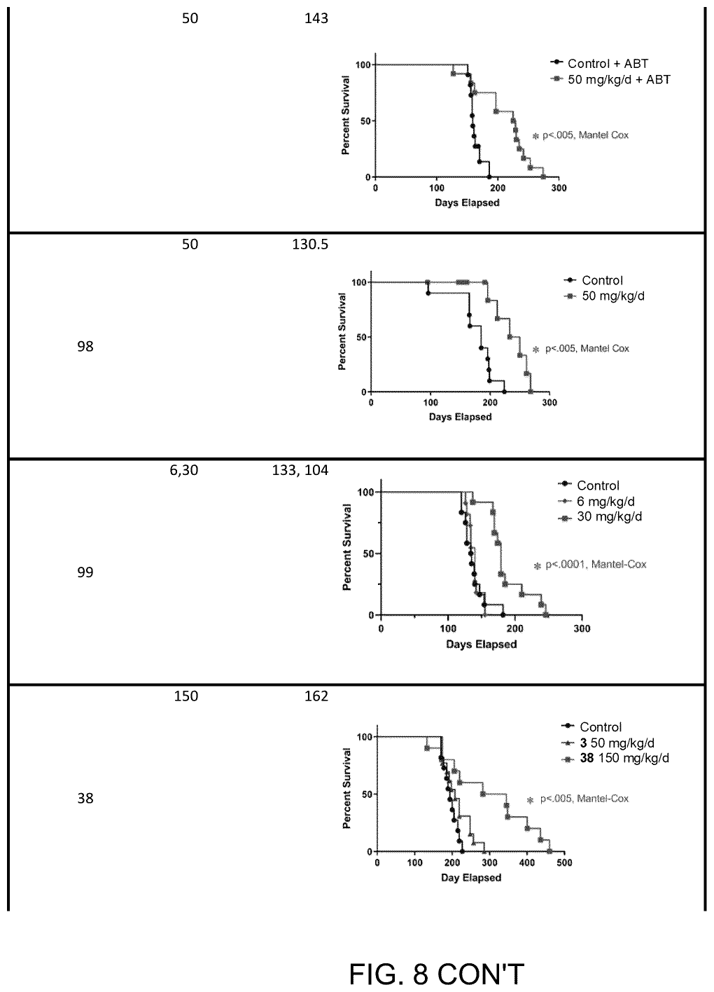

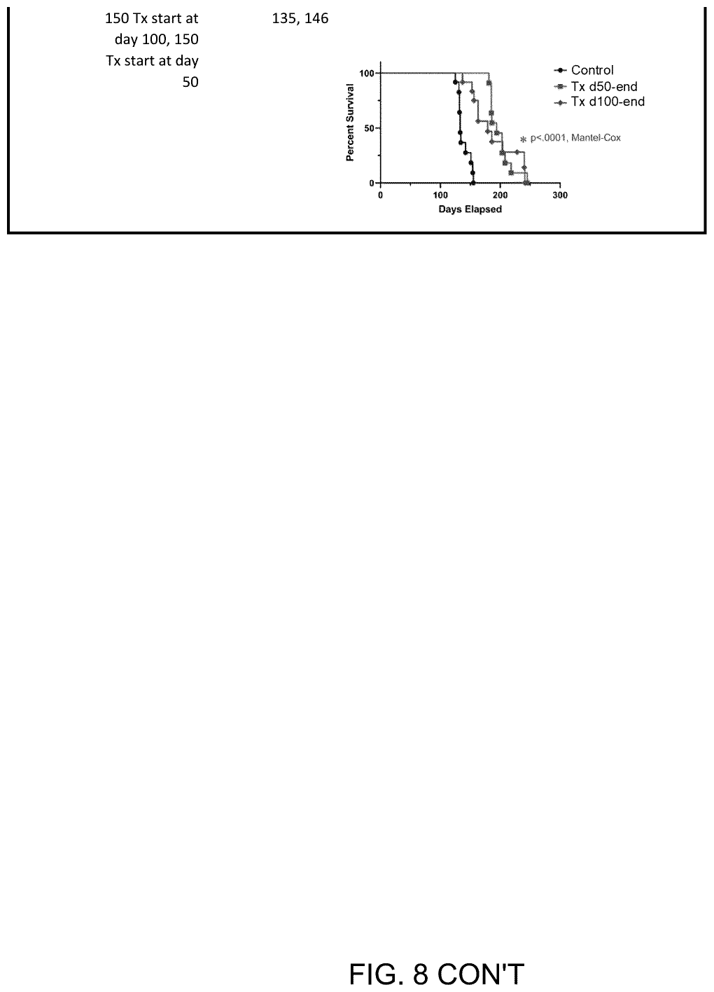

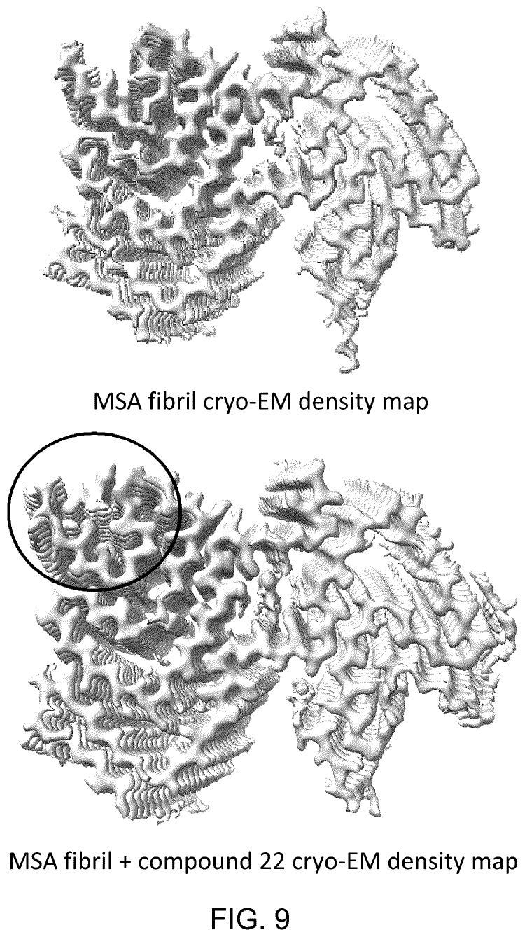

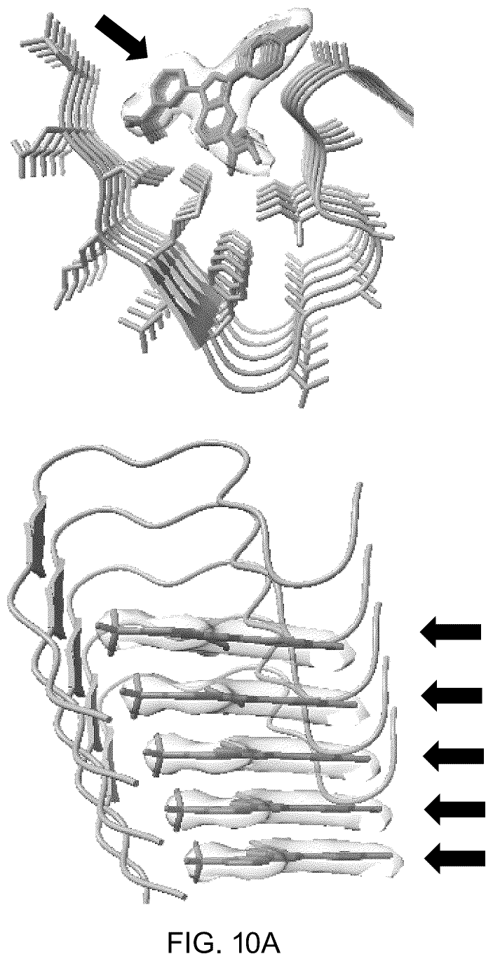

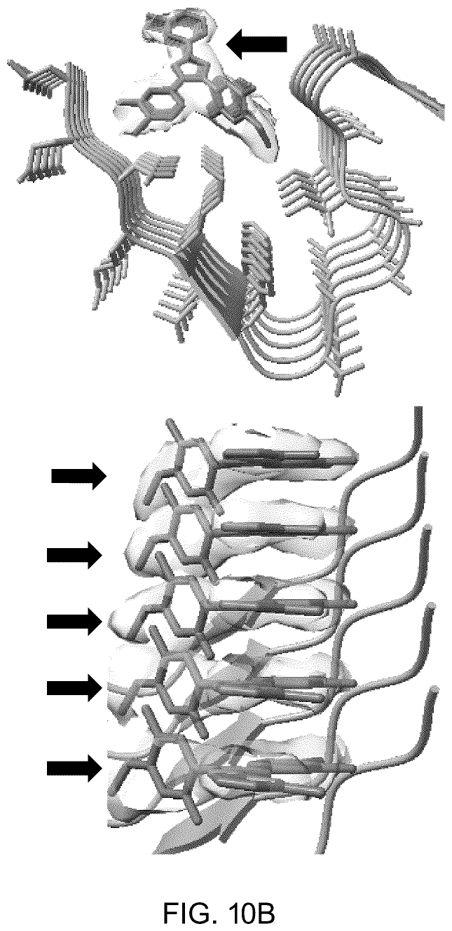

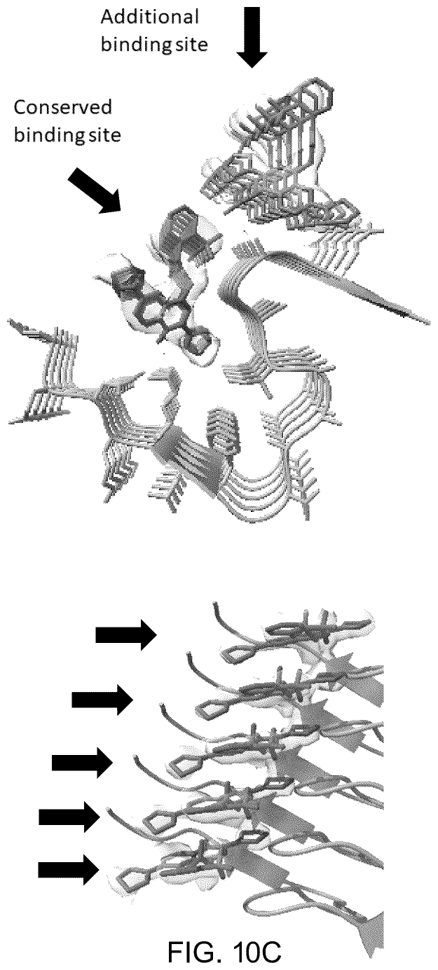

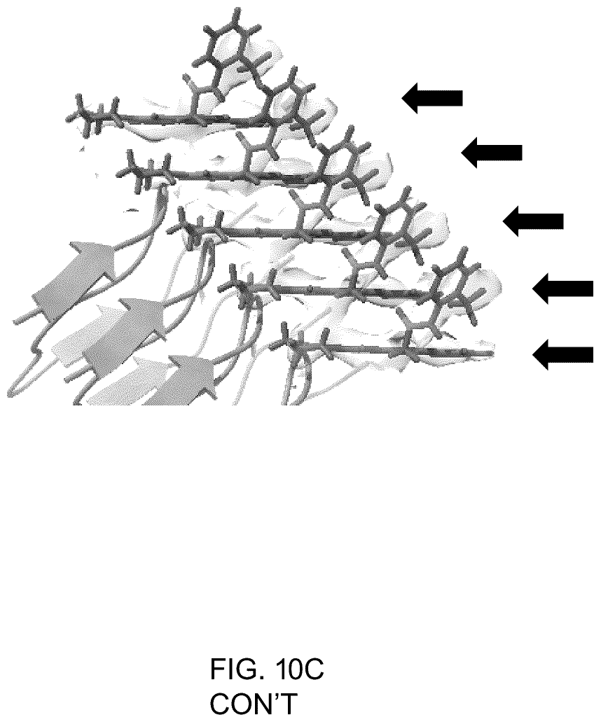

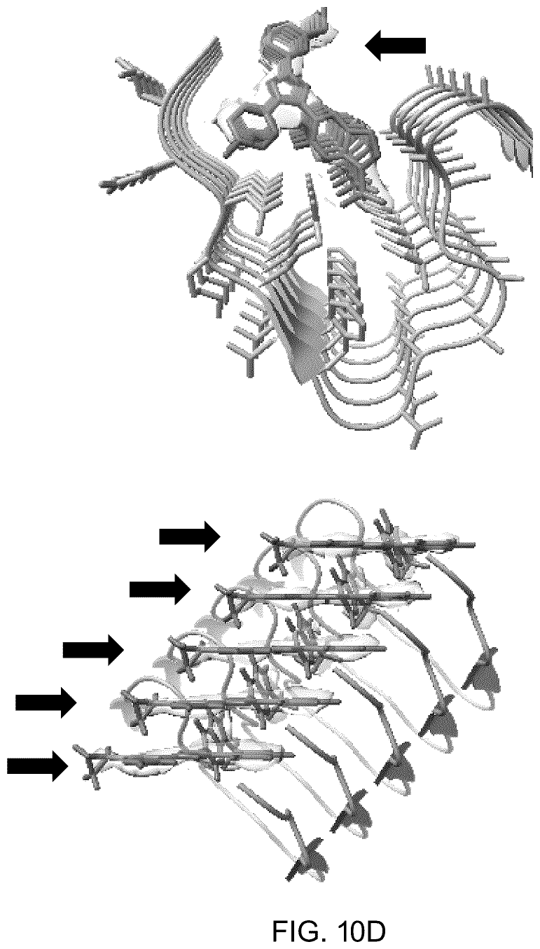

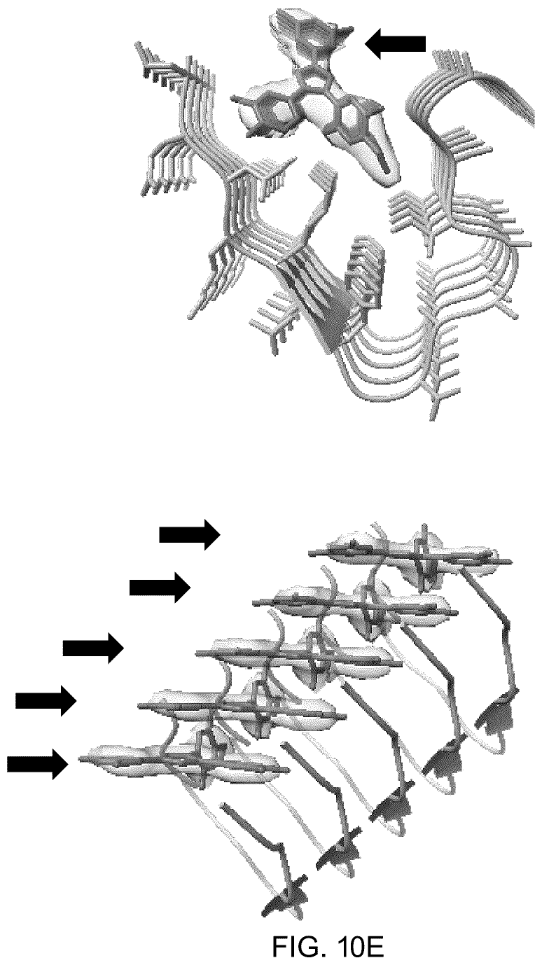

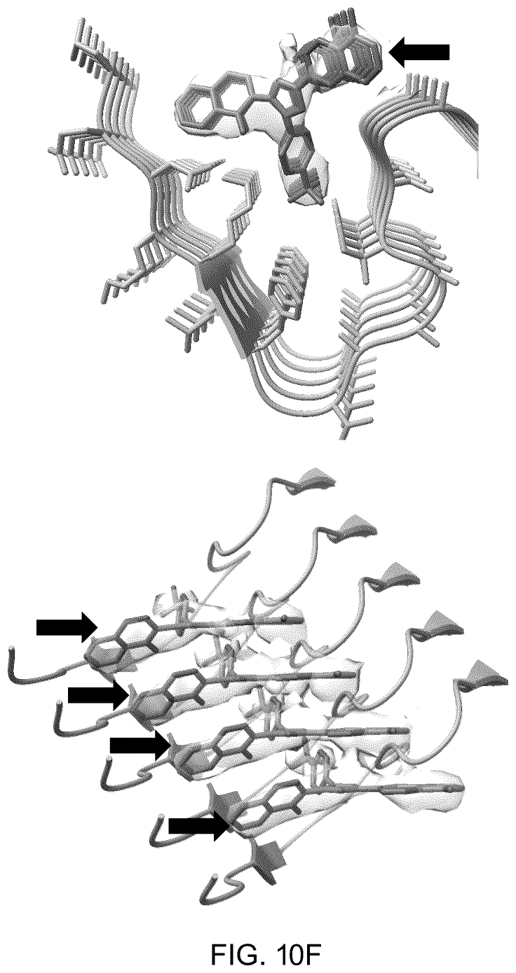

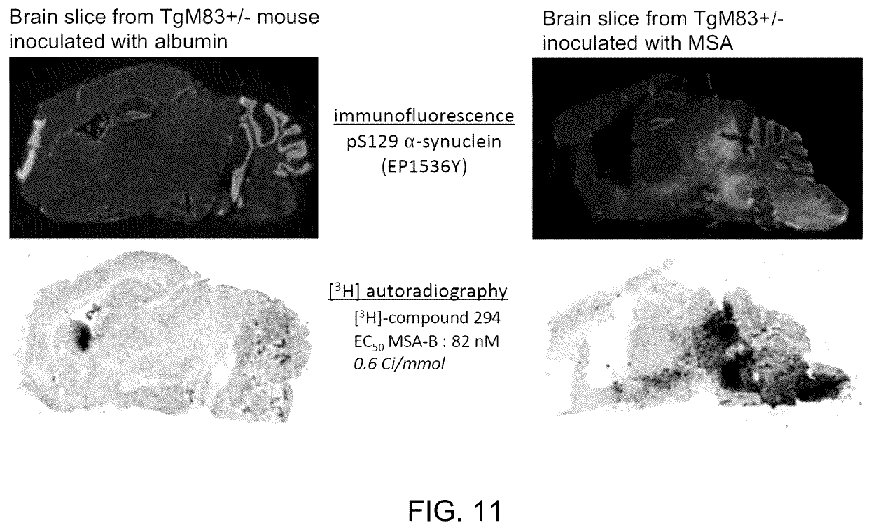

In one aspect, the invention provides a compound of the invention. In an exemplary embodiment, the invention is a compound described herein. In an exemplary embodiment, the invention is a compound according to a formula described herein. In some embodiments, the invention is any of the compounds disclosed in FIG. 6. In some embodiments, the invention is any of the compounds disclosed in FIG. 6, or a pharmaceutically acceptable salt or a hydrate or a solvate thereof.

In one aspect, the invention provides a compound, or a salt or a hydrate or a solvate thereof, having a structure according to formula (I):

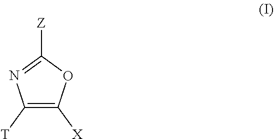

wherein T is substituted or unsubstituted naphthyridinone or substituted or unsubstituted dihydronaphthyridinone; X is substituted or unsubstituted aryl or substituted or unsubstituted heteroaryl, wherein X is monocyclic; and Z is substituted or unsubstituted aryl, substituted or unsubstituted heteroaryl, substituted or unsubstituted cycloalkyl, substituted or unsubstituted heterocycloalkyl, or substituted or unsubstituted ethenyl.

### Group T

In an exemplary embodiment, the compound is according to Formula (I), wherein X and Z are as described herein, and T is

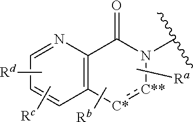

wherein R^{a}, R^{b}, R^{c}, and R^{d} are independently selected from H, halogen, substituted or unsubstituted C₁₋₃ alkyl, C₂-C₄ alkenyl, substituted or unsubstituted C₁₋₃ alkoxy, and when the connection between C* and C** is a single bond, R^{a} and R^{b} can be optionally joined with C* or with C** to form a substituted or unsubstituted cyclopropyl.

In an exemplary embodiment, the compound is according to Formula (I), wherein X and Z are as described herein, and T is

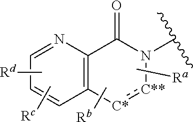

wherein R^{a}, R^{b}, R^{c}, and R^{d} are each independently selected from the group consisting of H, halogen, unsubstituted C₁₋₃ alkyl, C₁₋₃ alkyl substituted with one or more halogen, C₂-C₄ alkenyl, unsubstituted C₁₋₃ alkoxy, and C₁₋₃ alkoxy substituted with one or more halogen. In an exemplary embodiment, the compound is according to Formula (I), wherein X and Z are as described herein, and T is

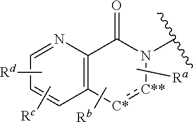

wherein R^{a}, R^{b}, R^{c}, and R^{d} are independently selected from the group consisting of H, F, Cl, Br, methyl, ethenyl, isopropyl, difluoromethyl, trifluoromethyl, 1,1-difluoroethyl, methoxy, trifluoromethoxy, difluoromethoxy, and 1,1-difluoroethoxy. In an exemplary embodiment, the compound is according to Formula (I), wherein X and Z are as described herein, and T is

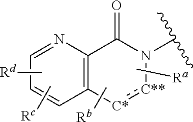

wherein R^{a}, R^{b}, R^{c}, and R^{d} are independently selected from the group consisting of H, F, Cl, Br, methyl, and methoxy.

In an exemplary embodiment, the compound is according to Formula (I), wherein X and Z are as described herein, and T is

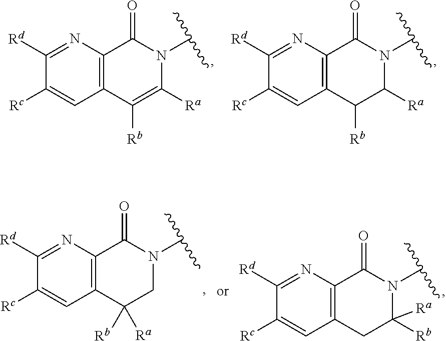

wherein R^{a}, R^{b}, R^{c}, and R^{d} are as described herein.

In an exemplary embodiment, the compound, or a salt, hydrate, or solvate thereof, is according to Formula (I), wherein X and Z are as described herein, and T is

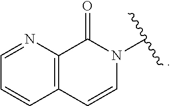 [SMILES: *n1ccc2cccnc2c1=O]

In an exemplary embodiment, the compound, or a salt, hydrate, or solvate thereof, is according to Formula (I), wherein X and Z are as described herein, and T is

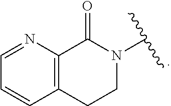 [SMILES: *N1CCc2cccnc2C1=O]

In an exemplary embodiment, the compound, or a salt, hydrate, or solvate thereof, is according to Formula (I), wherein X and Z are as described herein, and T is

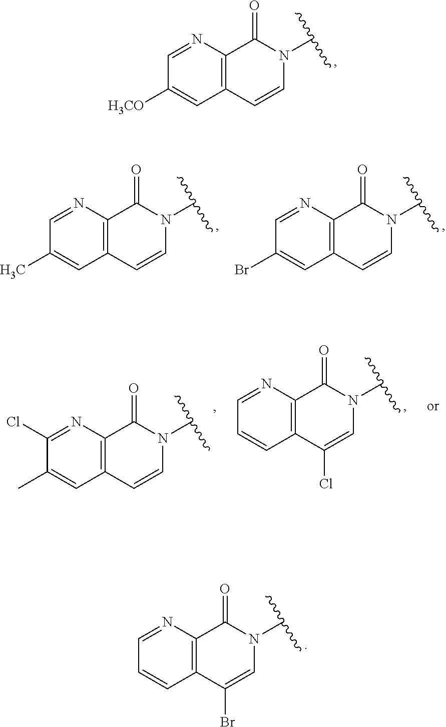 [SMILES: *n1ccc2cc(OC)cnc2c1=O ; *n1ccc2cc(C)cnc2c1=O ; *n1ccc2cc(Br)cnc2c1=O ; *n1ccc2cc(C)c(Cl)nc2c1=O ; *n1cc(Cl)c2cccnc2c1=O ; *n1cc(Br)c2cccnc2c1=O]

In an exemplary embodiment, the compound, or a salt, hydrate, or solvate thereof, is according to Formula (I), wherein X and Z are as described herein, and T is

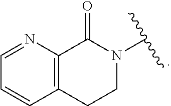 [SMILES: *N1CCc2cccnc2C1=O]

In an exemplary embodiment, the compound, or a salt, hydrate, or solvate thereof, is according to Formula (I), wherein X and Z are as described herein, and T is

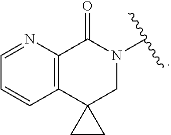 [SMILES: *N1CC2(CC2)c2cccnc2C1=O]

### Group X

In an exemplary embodiment, the compound is according to Formula (I), wherein T and Z are as described herein, and X is substituted or unsubstituted phenyl, substituted or unsubstituted pyridinyl, substituted or unsubstituted pyrimidinyl, or substituted or unsubstituted thienyl.

In an exemplary embodiment, the compound is according to Formula (I), wherein T and Z are as described herein, and X is substituted or unsubstituted phenyl. In an exemplary embodiment, the compound is according to Formula (I), wherein T and Z are as described herein, and X is unsubstituted phenyl or phenyl substituted with one or more members selected from the group consisting of halogen, C₂-C₄ alkenyl, unsubstituted C₁₋₃ alkyl, C₁₋₃ alkyl substituted with one or more halogen, unsubstituted C₁₋₃ alkoxy, and C₁₋₃ alkoxy substituted with one or more halogen. In an exemplary embodiment, the compound is according to Formula (I), wherein T and Z are as described herein, and X is unsubstituted phenyl or phenyl substituted with one or more members selected from the group consisting of F, Cl, Br, methyl, ethenyl, isopropyl, difluoromethyl, trifluoromethyl, 1,1-difluoroethyl, methoxy, trifluoromethoxy, difluoromethoxy, and 1,1-difluoroethoxy. In an exemplary embodiment, the compound is according to Formula (I), wherein T and Z are as described herein, and X is unsubstituted phenyl. In an exemplary embodiment, the compound is according to Formula (I), wherein T and Z are as described herein, and X is according to:

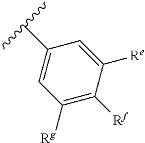

wherein R^{e}, R^{f}, and R^{g} are each independently selected from the group consisting of H, halogen, C₂-C₄ alkenyl, unsubstituted C₁₋₃ alkyl, C₁₋₃ alkyl substituted with one or more halogen, unsubstituted C₁₋₃ alkoxy, and C₁₋₃ alkoxy substituted with one or more halogen, wherein at least one of R^{e}, R^{f}, and R^{g} is not H. In an exemplary embodiment, the compound is according to Formula (I), wherein T and Z are as described herein, and X is according to:

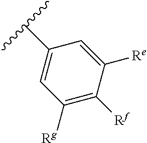

wherein R^{e}, R^{f}, and R^{g} are each independently selected from the group consisting of H, F, Cl, Br, methyl, ethenyl, isopropyl, difluoromethyl, trifluoromethyl, 1,1-difluoroethyl, methoxy, trifluoromethoxy, difluoromethoxy, and 1,1-difluoroethoxy, wherein at least one of R^{e}, R^{f}, and R^{g} is not H. In an exemplary embodiment, the compound is according to Formula (I), wherein T and Z are as described herein, and X is according to:

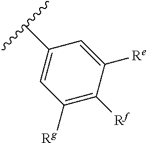

wherein R^{e}, R^{f}, and R^{g} are each independently selected from the group consisting of H, F, Cl, methyl, isopropyl, trifluoromethyl, trifluoromethoxy, and difluoromethoxy, wherein at least one of R^{e}, R^{f}, and R^{g} is not H.

In an exemplary embodiment, the compound is according to Formula (I), wherein T and Z are as described herein, and X is substituted or unsubstituted pyridinyl. In an exemplary embodiment, the compound is according to Formula (I), wherein T and Z are as described herein, and X is unsubstituted pyridinyl or pyridinyl substituted with one or more members selected from the group consisting of halogen, C₂-C₄ alkenyl, unsubstituted C₁₋₃ alkyl, C₁₋₃ alkyl substituted with one or more halogen, unsubstituted C₁₋₃ alkoxy, and C₁₋₃ alkoxy substituted with one or more halogen. In an exemplary embodiment, the compound is according to Formula (I), wherein T and Z are as described herein, and X is unsubstituted pyridinyl or pyridinyl substituted with one or more members selected from the group consisting of F, Cl, Br, methyl, ethenyl, isopropyl, difluoromethyl, trifluoromethyl, 1,1-difluoroethyl, methoxy, trifluoromethoxy, difluoromethoxy, and 1,1-difluoroethoxy. In an exemplary embodiment, the compound is according to Formula (I), wherein T and Z are as described herein, and X is unsubstituted pyridinyl. In an exemplary embodiment, the compound is according to Formula (I), wherein T and Z are as described herein, and X is according to:

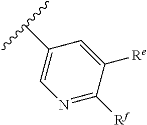

wherein R^{e} and R^{f} are each independently selected from the group consisting of H, halogen, C₂-C₄ alkenyl, unsubstituted C₁₋₃ alkyl, C₁₋₃ alkyl substituted with one or more halogen, unsubstituted C₁₋₃ alkoxy, and C₁₋₃ alkoxy substituted with one or more halogen, wherein at least one of R^{e} and R^{f} is not H. In an exemplary embodiment, the compound is according to Formula (I), wherein T and Z are as described herein, and X is according to:

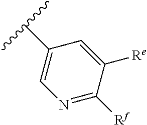

wherein R^{e} and R^{f} are each independently selected from the group consisting of H, F, Cl, Br, methyl, ethenyl, isopropyl, difluoromethyl, trifluoromethyl, 1,1-difluoroethyl, methoxy, trifluoromethoxy, difluoromethoxy, and 1,1-difluoroethoxy, wherein at least one of R^{e} and R^{f} is not H. In an exemplary embodiment, the compound is according to Formula (I), wherein T and Z are as described herein, and X is according to:

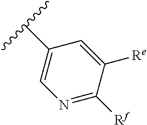

wherein R^{e} and R^{f} are each independently selected from the group consisting of H, F, methyl, ethenyl, isopropyl, trifluoromethyl, and 1,1-difluoroethyl, wherein at least one of R^{e} and R^{f} is not H.

In an exemplary embodiment, the compound is according to Formula (I), wherein T and Z are as described herein, and X is according to:

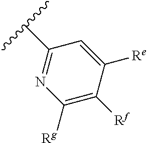

wherein R^{e}, R^{f}, and R^{g} are each independently selected from the group consisting of H, halogen, C₂-C₄ alkenyl, unsubstituted C₁₋₃ alkyl, C₁₋₃ alkyl substituted with one or more halogen, unsubstituted C₁₋₃ alkoxy, and C₁₋₃ alkoxy substituted with one or more halogen, wherein at least one of R^{e}, R^{f}, and R^{g} is not H. In an exemplary embodiment, the compound is according to Formula (I), wherein T and Z are as described herein, and X is according to:

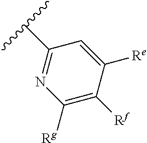

wherein R^{e}, R^{f}, and R^{g} are each independently selected from the group consisting of H, F, Cl, Br, methyl, ethenyl, isopropyl, difluoromethyl, trifluoromethyl, 1,1-difluoroethyl, methoxy, trifluoromethoxy, difluoromethoxy, and 1,1-difluoroethoxy, wherein at least one of R^{e}, R^{f}, and R^{g} is not H. In an exemplary embodiment, the compound is according to Formula (I), wherein T and Z are as described herein, and X is according to:

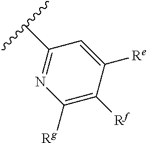

wherein R^{e}, R^{f}, and R^{g} are each independently selected from the group consisting of H, C₁, and trifluoromethyl, wherein at least one of R^{e}, R^{f}, and R^{g} is not H.

In an exemplary embodiment, the compound is according to Formula (I), wherein T and Z are as described herein, and X is substituted or unsubstituted pyrimidinyl. In an exemplary embodiment, the compound is according to Formula (I), wherein T and Z are as described herein, and X is unsubstituted pyrimidinyl or pyrimidinyl substituted with one or more members selected from the group consisting of halogen, C₂-C₄ alkenyl, unsubstituted C₁₋₃ alkyl, C₁₋₃ alkyl substituted with one or more halogen, unsubstituted C₁₋₃ alkoxy, and C₁₋₃ alkoxy substituted with one or more halogen. In an exemplary embodiment, the compound is according to Formula (I), wherein T and Z are as described herein, and X is unsubstituted pyrimidinyl or pyrimidinyl substituted with one or more members selected from the group consisting of F, Cl, Br, methyl, ethenyl, isopropyl, difluoromethyl, trifluoromethyl, 1,1-difluoroethyl, methoxy, trifluoromethoxy, difluoromethoxy, and 1,1-difluoroethoxy. In an exemplary embodiment, the compound is according to Formula (I), wherein T and Z are as described herein, and X is unsubstituted pyrimidinyl. In an exemplary embodiment, the compound is according to Formula (I), wherein T and Z are as described herein, and X is according to:

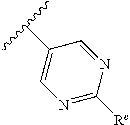

wherein R^{e} is H, halogen, C₂-C₄ alkenyl, unsubstituted C₁₋₃ alkyl, C₁₋₃ alkyl substituted with one or more halogen, unsubstituted C₁₋₃ alkoxy, or C₁₋₃ alkoxy substituted with one or more halogen. In an exemplary embodiment, the compound is according to Formula (I), wherein T and Z are as described herein, and X is according to:

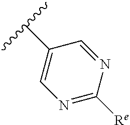

wherein R^{e} is F, Cl, Br, methyl, ethenyl, isopropyl, difluoromethyl, trifluoromethyl, 1,1-difluoroethyl, methoxy, trifluoromethoxy, difluoromethoxy, or 1,1-difluoroethoxy. In an exemplary embodiment, the compound is according to Formula (I), wherein T and Z are as described herein, and X is according to:

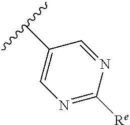

wherein R^{e} is trifluoromethyl.

In an exemplary embodiment, the compound is according to Formula (I), wherein T and Z are as described herein, and X is substituted or unsubstituted thienyl. In an exemplary embodiment, the compound is according to Formula (I), wherein T and Z are as described herein, and X is unsubstituted thienyl or thienyl substituted with one or more members selected from the group consisting of halogen, C₂-C₄ alkenyl, unsubstituted C₁₋₃ alkyl, C₁₋₃ alkyl substituted with one or more halogen, unsubstituted C₁₋₃ alkoxy, and C₁₋₃ alkoxy substituted with one or more halogen. In an exemplary embodiment, the compound is according to Formula (I), wherein T and Z are as described herein, and X is unsubstituted thienyl or thienyl substituted with one or more members selected from the group consisting of F, Cl, Br, methyl, ethenyl, isopropyl, difluoromethyl, trifluoromethyl, 1,1-difluoroethyl, methoxy, trifluoromethoxy, difluoromethoxy, and 1,1-difluoroethoxy. In an exemplary embodiment, the compound is according to Formula (I), wherein T and Z are as described herein, and X is unsubstituted thienyl. In an exemplary embodiment, the compound is according to Formula (I), wherein T and Z are as described herein, and X is according to:

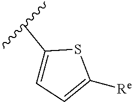

wherein R^{e} is halogen, C₂-C₄ alkenyl, unsubstituted C₁₋₃ alkyl, C₁₋₃ alkyl substituted with one or more halogen, unsubstituted C₁₋₃ alkoxy, or C₁₋₃ alkoxy substituted with one or more halogen. In an exemplary embodiment, the compound is according to Formula (I), wherein T and Z are as described herein, and X is according to:

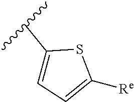

wherein R^{e} is F, Cl, Br, methyl, ethenyl, isopropyl, difluoromethyl, trifluoromethyl, 1,1-difluoroethyl, methoxy, trifluoromethoxy, difluoromethoxy, or 1,1-difluoroethoxy. In an exemplary embodiment, the compound is according to Formula (I), wherein T and Z are as described herein, and X is according to:

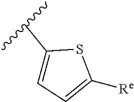

wherein R^{e} is trifluoromethyl.

In an exemplary embodiment, the compound, or a salt or a hydrate or a solvate thereof, has a structure according to formula (I), wherein T and Z are as described herein, and X is 4-(trifluoromethyl)phenyl, 4-fluorophenyl, 2-(trifluoromethyl)pyridin-5-yl, or 2-(1,1-difluoroethyl)pyridin-5-yl.

Group Z:

In an exemplary embodiment, the compound, or a salt or a hydrate or a solvate thereof, has a structure according to formula (I), wherein T and X are as described herein, and Z is substituted or unsubstituted aryl, substituted or unsubstituted heteroaryl, substituted or unsubstituted cycloalkyl, substituted or unsubstituted heterocycloalkyl, or substituted or unsubstituted ethenyl.

Part a

In an exemplary embodiment, the compound, or a salt or a hydrate or a solvate thereof, has a structure according to formula (I), wherein T and X are as described herein, and Z is substituted or unsubstituted phenyl, substituted or unsubstituted pyridinyl, substituted or unsubstituted furan, substituted or unsubstituted thienyl, substituted or unsubstituted pyrimidinyl, substituted or unsubstituted oxazolyl, or substituted or unsubstituted ethenyl.

In an exemplary embodiment, the compound, or a salt or a hydrate or a solvate thereof, has a structure according to formula (I), wherein T and X are as described herein, and Z is phenyl, pyridinyl, furan, thienyl, pyrimidinyl, or oxazolyl.

In an exemplary embodiment, the compound, or a salt or a hydrate or a solvate thereof, has a structure according to formula (I), wherein T and X are as described herein, and Z is ethenyl, substituted with pyridinyl or phenyl.

In an exemplary embodiment, the compound, or a salt or a hydrate or a solvate thereof, has a structure according to formula (I), wherein T and X are as described herein, and Z is substituted or unsubstituted 4-pyrimidinyl, substituted or unsubstituted 2-pyrimidinyl, substituted or unsubstituted 6-isoquinolyl, substituted or unsubstituted 3-quinolyl, substituted or unsubstituted 6-quinolyl, substituted or unsubstituted 1-pyrrolidinyl, substituted or unsubstituted 2,3-dihydro-1-benzofuran-5-yl, substituted or unsubstituted xylyl, substituted or unsubstituted tolyl, substituted or unsubstituted 4-mesylphenyl, substituted or unsubstituted 2-pyridinyl, substituted or unsubstituted 3-pyridinyl, substituted or unsubstituted 4-pyridinyl, substituted or unsubstituted 2,4-diaza-2-indanyl, substituted or unsubstituted 1,6-diaza-2-naphthyl, substituted or unsubstituted 1,5-diaza-2-naphthyl, substituted or unsubstituted 2,3-dihydro-1,4-dioxa-5-aza-7-naphthyl, substituted or unsubstituted 2,3-dihydro-1,4-benzodioxin-6-yl, substituted or unsubstituted 1-oxa-4-aza-2-indenyl, substituted or unsubstituted 6-quinoxalinyl, substituted or unsubstituted 2-quinoxalinyl, substituted or unsubstituted 2-furanyl, substituted or unsubstituted 1-oxa-6-aza-2-indenyl, substituted or unsubstituted 1,7a-diaza-2-indenyl, substituted or unsubstituted 1,7a-diaza-2-indenyl, substituted or unsubstituted 1,7a-diaza-2-indenyl, substituted or unsubstituted 1,3a-diaza-2-indenyl, substituted or unsubstituted 1-oxa-3,4-diaza-2-indenyl, substituted or unsubstituted 1,3a,6-triaza-2-indenyl, substituted or unsubstituted 1,3,3a-triaza-2-indenyl, substituted or unsubstituted 1,3a-diaza-2-indenyl, substituted or unsubstituted 1-oxa-4-aza-2-indenyl, substituted or unsubstituted 1,3a-diaza-3-indenyl, substituted or unsubstituted 1,3-oxazol-2-yl, substituted or unsubstituted 1,3-benzoxazol-2-yl, substituted or unsubstituted 5-benzothiophenyl, substituted or unsubstituted 1-pyrrolyl, substituted or unsubstituted 1-pyrazolyl, substituted or unsubstituted 1H-1,3-benzimidazol-1-yl, substituted or unsubstituted 2H-indazol-2-yl, substituted or unsubstituted 2H-indazol-5-yl, substituted or unsubstituted 1H-indazol-5-yl, substituted or unsubstituted indazol-5-yl, substituted or unsubstituted 2H-indazol-6-yl, substituted or unsubstituted 2H-1,2,7-triazainden-5-yl, substituted or unsubstituted 2H-1,2,4-triazainden-5-yl, substituted or unsubstituted 2H-1,2,6-triazainden-5-yl, substituted or unsubstituted 1-benzofuran-2-yl, substituted or unsubstituted 2,3-dihydro-1-benzofuran-4-yl, substituted or unsubstituted 1-benzofuran-4-yl, substituted or unsubstituted 1-benzofuran-6-yl, substituted or unsubstituted 2,3-dihydro-1-benzofuran-7-yl, substituted or unsubstituted 1-benzofuran-7-yl, substituted or unsubstituted 2,4-diaza-2-indanyl, substituted or unsubstituted 2,5-diaza-2-indanyl, substituted or unsubstituted 2,4,7-triaza-2-indanyl, substituted or unsubstituted 3,4-dihydro-2H-1,4-benzoxazin-6-yl, substituted or unsubstituted 3,4-dihydro-2H-1,4-benzoxazin-7-yl, substituted or unsubstituted 1,3-thiazol-2-yl, substituted or unsubstituted 2-thienyl, substituted or unsubstituted 1,3-benzothiazol-2-yl, substituted or unsubstituted imidazolidinone, substituted or unsubstituted 2-isoindolinyl, substituted or unsubstituted 1-oxo-2-isoindolinyl, or substituted or unsubstituted 6-indolyl.

In an exemplary embodiment, the compound, or a salt or a hydrate or a solvate thereof, has a structure according to formula (I), wherein T and X are as described herein, and Z is substituted or unsubstituted 2-(pyridinyl)ethenyl, or substituted or unsubstituted 2-(phenyl)ethenyl. In an exemplary embodiment, the compound, or a salt or a hydrate or a solvate thereof, has a structure according to formula (I), wherein T and X are as described herein, and Z is substituted or unsubstituted 2-(4-pyridinyl)ethenyl, substituted or unsubstituted 2-(3-pyridinyl)ethenyl, or substituted or unsubstituted 2-(phenyl)ethenyl. In an exemplary embodiment, the compound, or a salt or a hydrate or a solvate thereof, has a structure according to formula (I), wherein T and X are as described herein, and Z is 2-(4-pyridinyl)ethenyl, 2-(3-pyridinyl)ethenyl, or 2-(phenyl)ethenyl. In an exemplary embodiment, the compound, or a salt or a hydrate or a solvate thereof, has a structure according to formula (I), wherein T and X are as described herein, and Z is 2-(4-pyridinyl)ethenyl substituted with halogen or C₁₋₃ alkyl, 2-(3-pyridinyl)ethenyl substituted with halogen or C₁₋₃ alkyl, or 2-(phenyl)ethenyl substituted with halogen or C₁₋₃ alkyl.

In an exemplary embodiment, the compound, or a salt or a hydrate or a solvate thereof, has a structure according to formula (I), wherein T and X are as described herein, and Z is

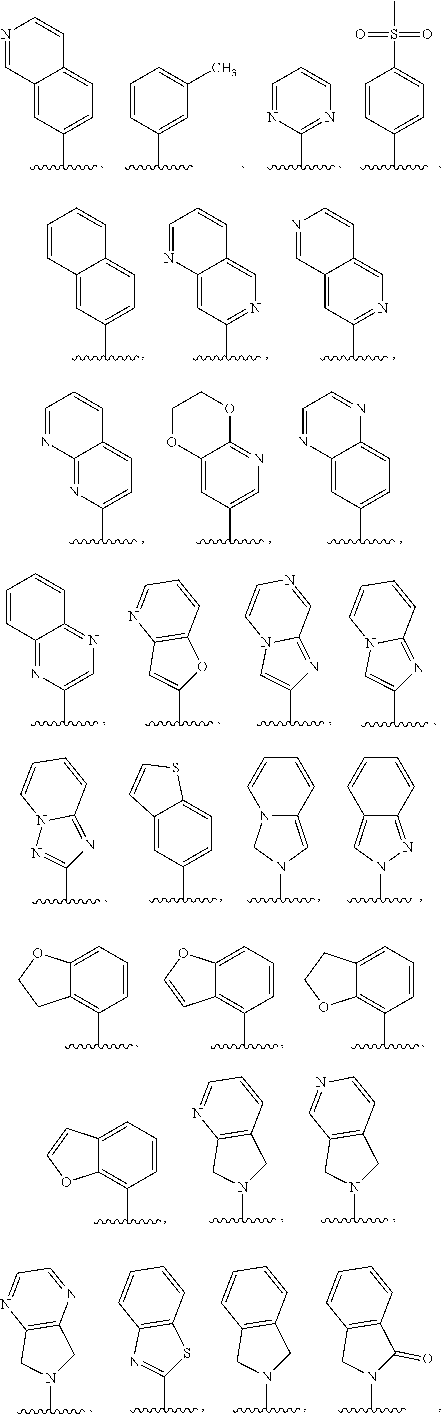

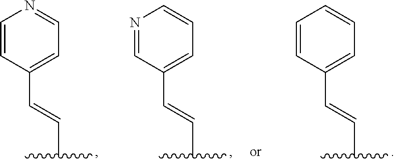 [SMILES: */C=C/c1ccncc1 ; */C=C/c1cccnc1 ; */C=C/c1ccccc1]

In an exemplary embodiment, the compound, or a salt or a hydrate or a solvate thereof, has a structure according to formula (I), wherein T and X are as described herein, and Z is

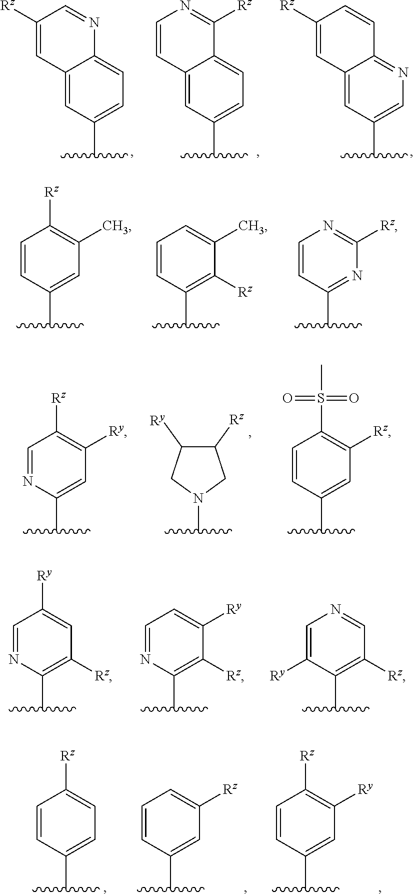

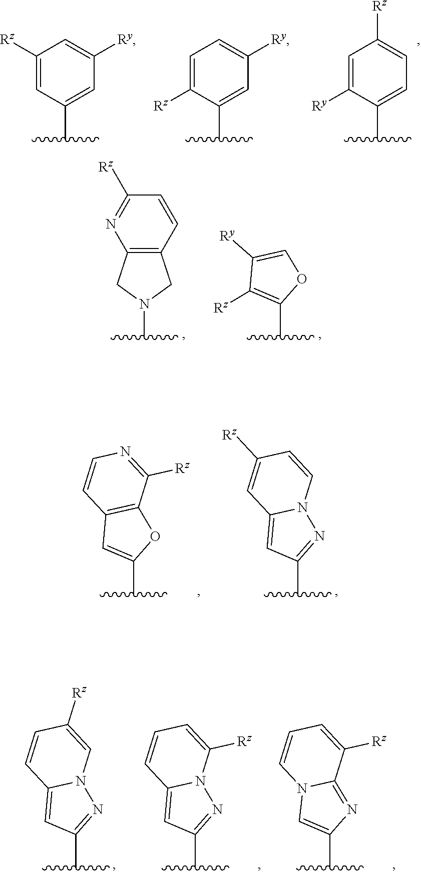

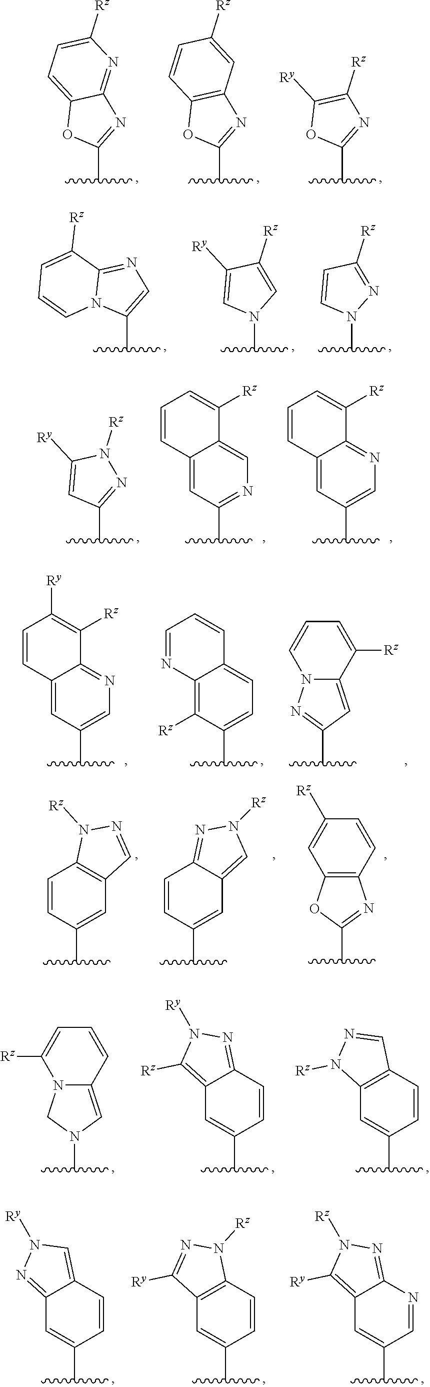

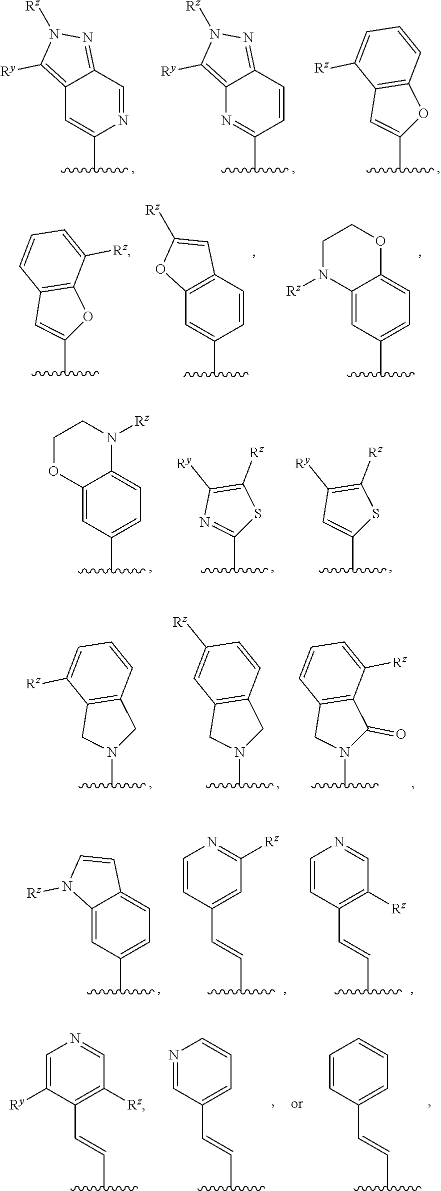

wherein R^{y} and R^{z} are each independently selected from H, halogen, cyano, cyclopropyl, C₁-C₃ alkyl, C₁-C₃ alkyl substituted with cyclopropyl, C₁-C₃ alkyl substituted with C₁-C₃ alkoxy, C₁-C₃ alkyl substituted with hydroxy, C₁-C₃ alkyl substituted with one or more halogen, C₁-C₃ alkyl substituted with hydroxy and one or more halogen, C₂-C₄ alkenyl, C₁-C₃ alkoxy, C₁-C₃ alkoxy substituted with cyclopropyl, C₁-C₃ alkoxy substituted with one or more hydroxy, C₁-C₃ alkoxy substituted with one or more halogen, C₁-C₃ alkoxy substituted with hydroxy and one or more halogen, C₁-C₃ alkylamino, C₁-C₃ dialkylamino, pyridinyl, pyridinyl substituted with C₁-C₃ alkyl, 1H-1,2,4-triazol-3-yl, 1-methyl-1H-1,2,4-triazol-3-yl substituted with C₁-C₃ alkyl, wherein at least one of R^{y} and R^{z} is not H.

In an exemplary embodiment, the compound, or a salt or a hydrate or a solvate thereof, has a structure according to formula (I), wherein T and X are as described herein, and Z is

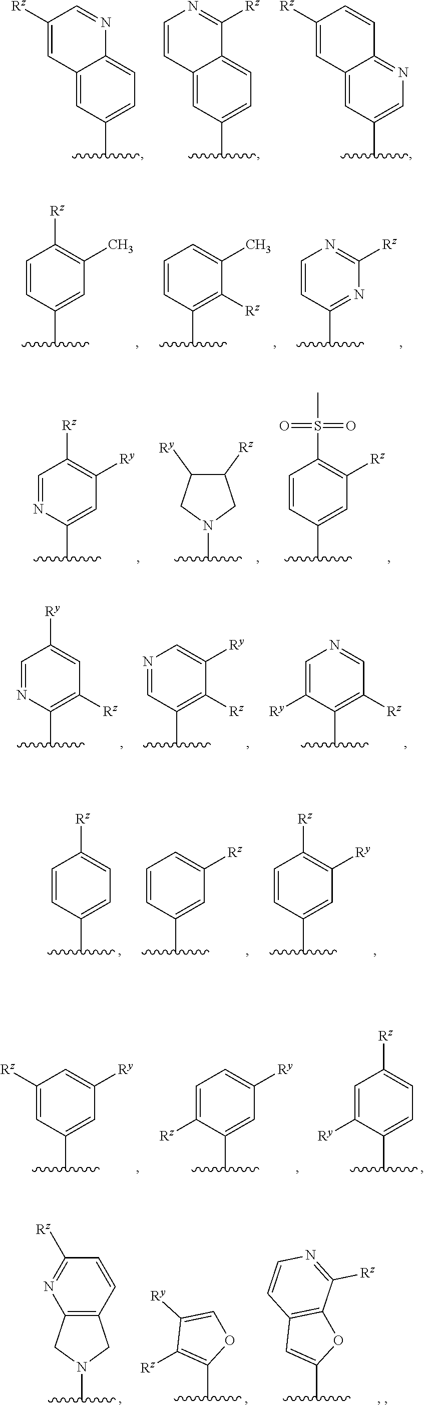

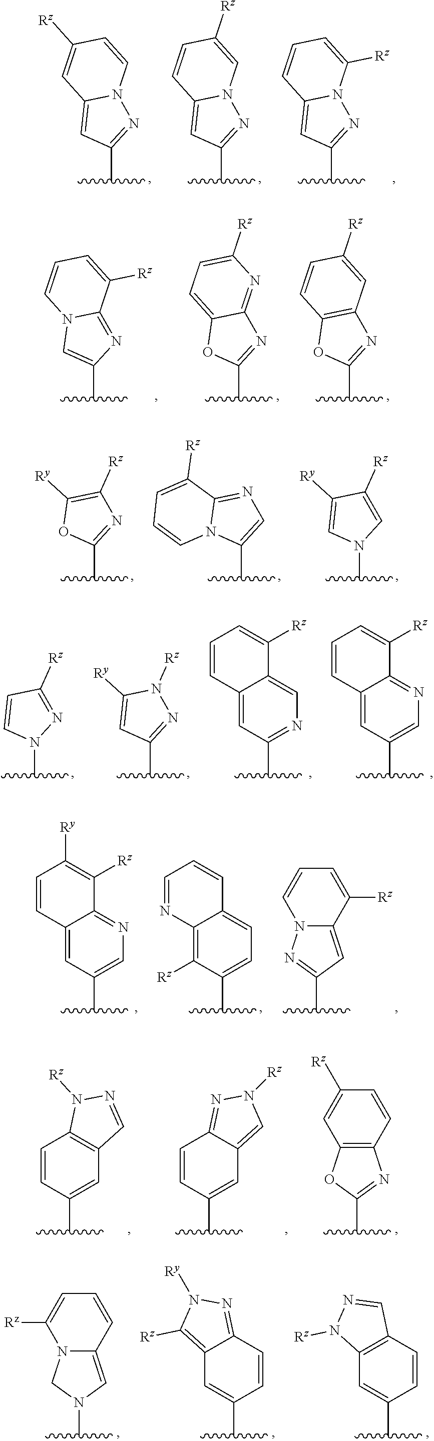

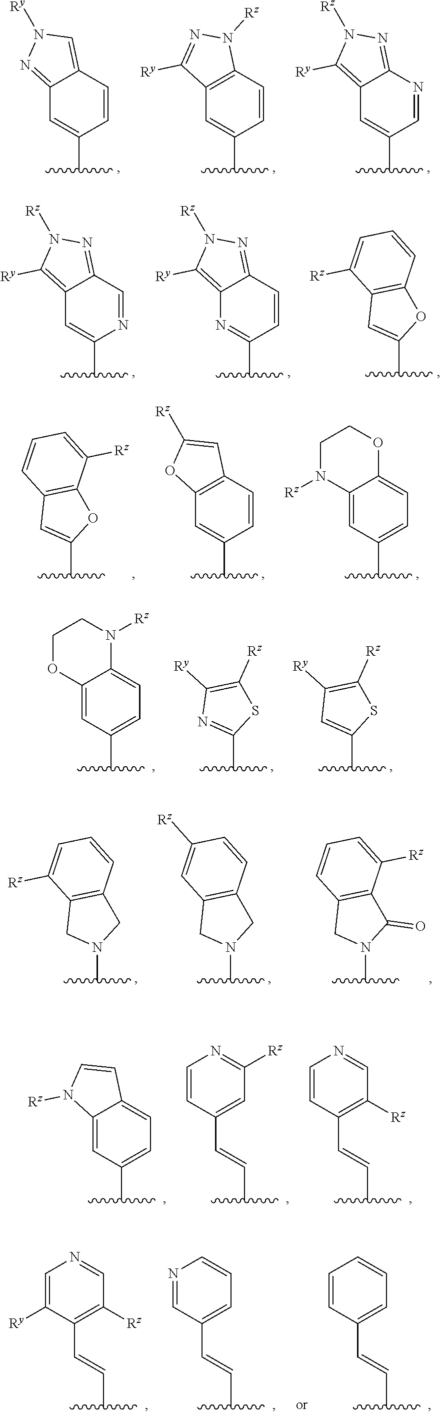

wherein R^{y} and R^{z} are each independently selected from H, F, Cl, cyano, cyclopropyl, methyl, cyclopropylmethyl, ethenyl, isopropyl, methoxy, 2-ethoxy, difluoromethyl, trifluoromethyl, trifluoromethoxy, difluoromethoxy, 3,3,3-trifluoro-2-hydroxypropyl, methoxymethyl, 2,2-difluoroethyl, dimethylamino, 2,2,2-trifluoroethyl, 2-hydroxyethyl, and 2-methyl-4-pyridyl, 1-methyl-1H-1,2,4-triazol-3-yl, wherein at least one of R^{y} and R^{z} is not H.

In an exemplary embodiment, the compound, or a salt or a hydrate or a solvate thereof, has a structure according to formula (I), wherein T and X are as described herein, and Z is 1-methyl-6-isoquinolyl, 1-methoxy-6-isoquinolyl, 3-chloro-6-isoquinolyl, m-tolyl, 2-fluoro-3-tolyl, 4-fluoro-3-tolyl, 2-ethoxy-4-pyrimidinyl, 4,5-dimethyl-2-pyridyl, p-(trifluoromethyl)phenyl, 2,3-dihydro-1-benzofuran-5-yl, (3S,4S)-3,4-dimethyl-1-pyrrolidinyl, 3-fluoro-4-mesylphenyl, 5,6-dimethyl-3-pyridyl, p-(1-methyl-1H-1,2,4-triazol-3-yl)phenyl, 4-cyano-phenyl, 3-chloro-4-methoxyphenyl, phenyl, 3,5-dichlorophenyl, 3-chloro-4-fluorophenyl, 5-chloro-2-fluorophenyl, m-chlorophenyl, p-chlorophenyl, p-fluorophenyl, 3-chloro-2-fluorophenyl, 2-fluoro-4-cyano-phenyl, p-difluoromethoxyphenyl, 2-fluoro-3-methoxyphenyl, p-methoxyphenyl, p-(dimethylamino)phenyl, 5-methyl-2,4-diaza-2-indanyl, p-mesylphenyl, 2-naphthyl, 1,5-diaza-2-naphthyl, 1,6-diaza-2-naphthyl, 1,8-diaza-2-naphthyl, 2,3-dihydro-1,4-dioxa-5-aza-7-naphthyl, 3-methoxy-6-quinolyl, 1-methyl-1,2,3,4-tetrahydro-6-quinolyl, 1-methyl-4,4-dimethyl-2-oxo-3,4-dihydro-6-quinolyl, 6-methyl-3-quinolyl, 6-fluoro-3-quinolyl, 7-(trifluoromethyl)-3-quinolyl, 2,6-dimethyl-4-pyridyl, 6-methoxy-5-methyl-3-pyridyl, 5-cyclopropyl-3-pyridinyl, 5-chloro-6-methyl-3-pyridinyl, 5-chloro-6-(methoxymethyl)-3-pyridinyl, 5-chloro-6-methoxy-3-pyridinyl, 6-(dimethylamino)-3-pyridinyl, 3-pyridinyl, 6-cyano-2-pyridinyl, 4-ethynyl-2-pyridinyl, 2-pyridinyl, 6-chloro-2-pyridinyl, 6-(trifluoromethyl)-2-pyridyl, 6-methyl-2-pyridinyl, 5-chloro-2-furanyl, 5-(trifluoromethyl)-2-furanyl, 4,5-dimethyl-2-furanyl, 6-quinoxalinyl, 2-quinoxalinyl, 7-methoxy-1-oxa-6-aza-2-indenyl, 6-chloro-1,7a-diaza-2-indenyl, 7-methyl-1,7a-diaza-2-indenyl, 5-methyl-1,7a-diaza-2-indenyl, 7-methyl-1,3a-diaza-2-indenyl, 5-methyl-1-oxa-3,4-diaza-2-indenyl, 1,3a,6-triaza-2-indenyl, 1,3,3a-triaza-2-indenyl, 1,3a-diaza-2-indenyl, 1-oxa-4-aza-2-indenyl, 7-methoxy-1,3a-diaza-3-indenyl, 5-cyclopropyl-4-methyl-1,3-oxazol-2-yl, 5-methoxy-1,3-benzoxazol-2-yl, 5-fluoro-1,3-benzoxazol-2-yl, 1,3-benzoxazol-2-yl, 5-cyano-benzoxazol-2-yl, 1-benzothiophen-5-yl, 3-bromo-4-methyl-1-pyrrolyl, 3-(trifluoromethyl)-1-pyrazolyl, 5-cyclopropyl-1-methyl-3-pyrazolyl, 1-(2,2-difluoroethyl)-5-methyl-3-pyrazolyl, 1-isopropyl-5-methyl-3-pyrazolyl, 4-methoxy-1H-1,3-benzimidazol-1-yl, 1H-1,3-benzimidazol-2-yl, 1-methyl-1H-1,3-benzimidazol-2-yl, 2H-indazol-2-yl, 3-(difluoromethyl)-2-methyl-2H-indazol-5-yl, 1-(2,2-difluoroethyl)-1H-indazol-5-yl, 1-(difluoromethyl)-1H-indazol-5-yl, 1-ethyl-3-methyl-1H-indazol-5-yl, 2-(3,3,3-trifluoro-2-hydroxypropyl)-2H-indazol-5-yl, 2-(2,2,2-trifluoroethyl)-2H-indazol-5-yl, 2-methylcyano-indazol-5-yl, 2-ethyl-3-methyl-2H-indazol-5-yl, 2-cyclobutyl-2H-indazol-5-yl, 2-isopropyl-2H-indazol-5-yl, 2-(cyclopropylmethyl)-2H-indazol-5-yl, 2-methyl-2H-indazol-6-yl, 2-methyl-3-methyl-2H-1,2,7-triazainden-5-yl, 2-methyl-2H-1,2,4-triazainden-5-yl, 2-methyl-3-methyl-2H-1,2,6-triazainden-5-yl, 4-fluoro-1-benzofuran-2-yl, 7-fluoro-1-benzofuran-2-yl, 7-methoxy-1-benzofuran-2-yl, 3-methyl-1-benzofuran-2-yl, 1-benzofuran-2-yl, 2,3-dihydro-1-benzofuran-4-yl, 2-methyl-1-benzofuran-6-yl, 2,3-dihydro-1-benzofuran-7-yl, 2,4-diaza-2-indanyl, 2,5-diaza-2-indanyl, 2,4,7-triaza-2-indanyl, 4-methyl-3,4-dihydro-2H-1,4-benzoxazin-6-yl, 4-methyl-3,4-dihydro-2H-1,4-benzoxazin-7-yl, 5-cyclopropyl-4-methyl-1,3-thiazol-2-yl, 4,5-dimethyl-1,3-thiazol-2-yl, 2-pyrimidinyl, 4-pyrimidinyl, 4,5-dimethyl-2-thienyl, 1,3-benzothiazol-2-yl, p-phenyl-2-imidazolidinone, 2-isoindolinyl, 7-fluoro-1-oxo-2-isoindolinyl, 1-oxo-2-isoindolinyl, 4-methoxy-2-isoindolinyl, 5-methoxy-2-isoindolinyl, or 1-methyl-6-indolyl.

In an exemplary embodiment, the compound, or a salt or a hydrate or a solvate thereof, has a structure according to formula (I), wherein T and X are as described herein, and Z is (E)-2-(3-fluoro-4-pyridyl)ethenyl, (E)-2-(2-fluoro-4-pyridyl)ethenyl, (E)-2-phenylethenyl, (E)-2-(2,6-dimethyl-4-pyridyl)ethenyl, (E)-2-(4-pyridyl)ethenyl, (E)-2-(2-methyl-4-pyridyl)ethenyl, or (E)-2-(3-pyridyl)ethenyl.

Part b

In an exemplary embodiment, the compound, or a salt or a hydrate or a solvate thereof, has a structure according to formula (I), wherein T and X are as described herein, and Z is substituted or unsubstituted phenyl, substituted or unsubstituted pyridinyl, substituted or unsubstituted furan, substituted or unsubstituted thienyl, substituted or unsubstituted pyrimidinyl, and substituted or unsubstituted oxazolyl.

In an exemplary embodiment, the compound, or a salt or a hydrate or a solvate thereof, has a structure according to formula (I), wherein T and X are as described herein, and Z is substituted or unsubstituted pyrimidinyl, substituted or unsubstituted isoquinolyl, substituted or unsubstituted quinolyl, substituted or unsubstituted xylyl, substituted or unsubstituted pyrrolyl, substituted or unsubstituted cyclopropyl, substituted or unsubstituted oxazolyl, substituted or unsubstituted triazaindenyl, substituted or unsubstituted diazaindenyl, substituted or unsubstituted phenyl, substituted or unsubstituted naphthyl, substituted or unsubstituted benzofuranyl, substituted or unsubstituted indazolyl, substituted or unsubstituted benzothiazolyl, substituted or unsubstituted benzoxazolyl, substituted or unsubstituted indolyl, substituted or unsubstituted 2,3-dihydro-1,4-benzodioxin-6-yl, substituted or unsubstituted thienyl, substituted or unsubstituted isoindoinlyl, or substituted or unsubstituted 1-oxa-4-aza-2-indenyl.

In an exemplary embodiment, the compound, or a salt or a hydrate or a solvate thereof, has a structure according to formula (I), wherein T and X are as described herein, and Z is

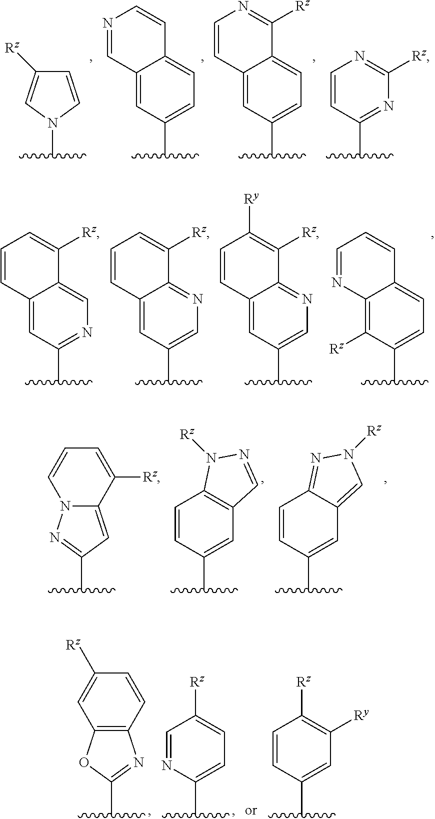

wherein R^{y} and R^{z} are each independently selected from H, halogen, cyano, cyclopropyl, C₁-C₃ alkyl, C₁-C₃ alkyl substituted with cyclopropyl, C₁-C₃ alkyl substituted with C₁-C₃ alkoxy, C₁-C₃ alkyl substituted with hydroxy, C₁-C₃ alkyl substituted with one or more halogen, C₁-C₃ alkyl substituted with hydroxy and one or more halogen, C₂-C₄ alkenyl, C₁-C₃ alkoxy, C₁-C₃ alkoxy substituted with cyclopropyl, C₁-C₃ alkoxy substituted with one or more hydroxy, C₁-C₃ alkoxy substituted with one or more halogen, C₁-C₃ alkoxy substituted with hydroxy and one or more halogen, C₁-C₃ alkylamino, C₁-C₃ dialkylamino, pyridinyl, pyridinyl substituted with C₁-C₃ alkyl, 1H-1,2,4-triazol-3-yl, 1-methyl-1H-1,2,4-triazol-3-yl substituted with C₁-C₃ alkyl, wherein at least one of R^{y} and R^{z} is not H.

In an exemplary embodiment, the compound, or a salt or a hydrate or a solvate thereof, has a structure according to formula (I), wherein T and X are as described herein, and Z is

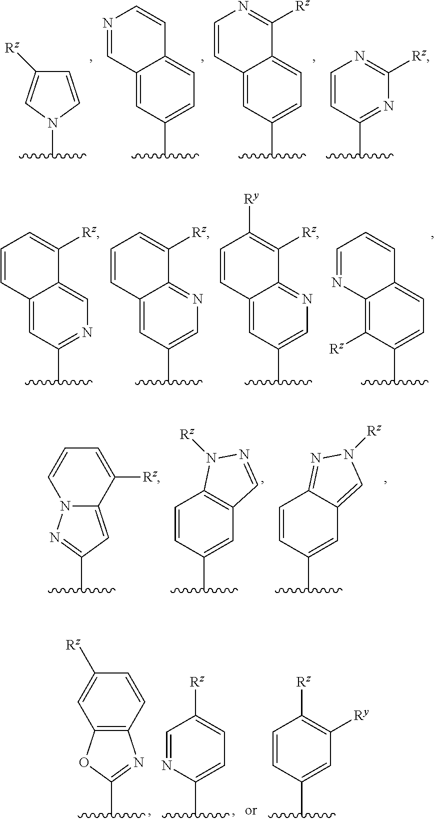

wherein R^{y} and R^{z} are each independently selected from H, fluoro, chloro, cyclopropyl, methyl, isopropyl, methoxy, 2-hydroxyethyl, and 2-methyl-4-pyridyl, wherein at least one of R^{y} and R^{z} is not H.

In an exemplary embodiment, the compound, or a salt or a hydrate or a solvate thereof, has a structure according to formula (I), wherein T and X are as described herein, and Z is 2-cyclopropyl-4-pyrimidinyl, 1-methyl-6-isoquinolyl, 1-(2-hydroxyethyl)-6-isoquinolyl, 7-isoquinolyl, 8-fluoro-3-quinolyl, 1-methoxy-6-isoquinolyl, 3,4-xylyl, 1-isopropyl-6-isoquinolyl, 8-fluoro-7-methyl-3-quinolyl, 3-(2-methoxy-3-pyridyl)-1-pyrrolyl, 2-cyclopropyl, 2-(2-methyl-4-pyridyl)-1,3-oxazol-5-yl, 2-methyl-6-quinolyl, 2-methyl-2H-1,2,7-triazainden-5-yl, p-phenyl-1,3-oxazolidin-2-one, 5-fluoro-1,3-benzoxazol-2-yl, 4-chloro-5-methoxy-1-benzofuran-2-yl, 6-isoquinolyl, 6-fluoro-1-methyl-1H-indazol-5-yl, 2-naphthyl, 2-methyl-1,3-benzoxazol-5-yl, 5-methoxy-1,3-benzothiazol-2-yl, 6-fluoro-1,3-benzoxazol-2-yl, 3-thia-1,4-diaza-2-indenyl, 2-indole-5-carbonitrile, 2,3-dihydro-1,4-benzodioxin-6-yl, 2-methyl-1,3-benzoxazol-6-yl, 2-fluoro-4-methoxyphenyl, 5-methoxy-1,3-benzoxazol-2-yl, 5-methoxy-2-isoindolinyl, 6-methoxy-1,3-benzothiazol-2-yl, 2-methyl-2H-indazol-5-yl, 1-oxa-4-aza-2-indenyl, 4-methoxy-2-isoindolinyl, 4-fluoro-5-methoxy-2-isoindolinyl, phenyl, p-(dimethylamino)phenyl, 1-benzofuran-5-yl, m-methoxyphenyl, 5-methyl-2-thienyl, p-methoxyphenyl, p-chlorophenyl.

Part c

In an exemplary embodiment, the compound, or a salt or a hydrate or a solvate thereof, has a structure according to formula (I), wherein T and X are as described herein, and Z is substituted or unsubstituted 6-isoquinolyl, substituted or unsubstituted 2-quinolyl, substituted or unsubstituted 3-quinolyl, substituted or unsubstituted 4-quinolyl, substituted or unsubstituted 6-quinolyl, substituted or unsubstituted 7-quinolyl, substituted or unsubstituted 7-quinazolinyl, substituted or unsubstituted 1H-indazol-5-yl, substituted or unsubstituted 2H-indazol-5-yl, substituted or unsubstituted 1-benzofuran-6-yl, substituted or unsubstituted 1-benzofuran-2-yl, substituted or unsubstituted 2,3-dihydro-1-benzofuran-6-yl, substituted or unsubstituted 1,2,3-benzotriazol-5-yl, substituted or unsubstituted benzothiazol-2-yl, substituted or unsubstituted 1,3a-diaza-2-indenyl, substituted or unsubstituted 1,7a-diaza-2-indenyl, substituted or unsubstituted 1,3a-diaza-5-indenyl, substituted or unsubstituted 3,4-diaza-2-indenyl, substituted or unsubstituted 1-thia-4-aza-2-indenyl, substituted or unsubstituted indazol-5-yl, substituted or unsubstituted 1H-indazol-5-yl, substituted or unsubstituted 2H-indazol-5-yl, substituted or unsubstituted 1,2,6-triazainden-5-yl, substituted or unsubstituted 1H-1,2,6-triazainden-5-yl, substituted or unsubstituted 2H-1,2,6-triazainden-5-yl, substituted or unsubstituted phenyl, substituted or unsubstituted 1-pyrrolyl, substituted or unsubstituted 2-indolyl, substituted or unsubstituted 5-indolinyl, substituted or unsubstituted 2-oxo-5-indolinyl, substituted or unsubstituted 2-isoindolinyl, substituted or unsubstituted 3-oxo-5-isoindolinyl, substituted or unsubstituted 1-oxo-5-isoindolinyl, or substituted or unsubstituted 2-thienyl.

In an exemplary embodiment, the compound, or a salt or a hydrate or a solvate thereof, has a structure according to formula (I), wherein T and X are as described herein, and Z is

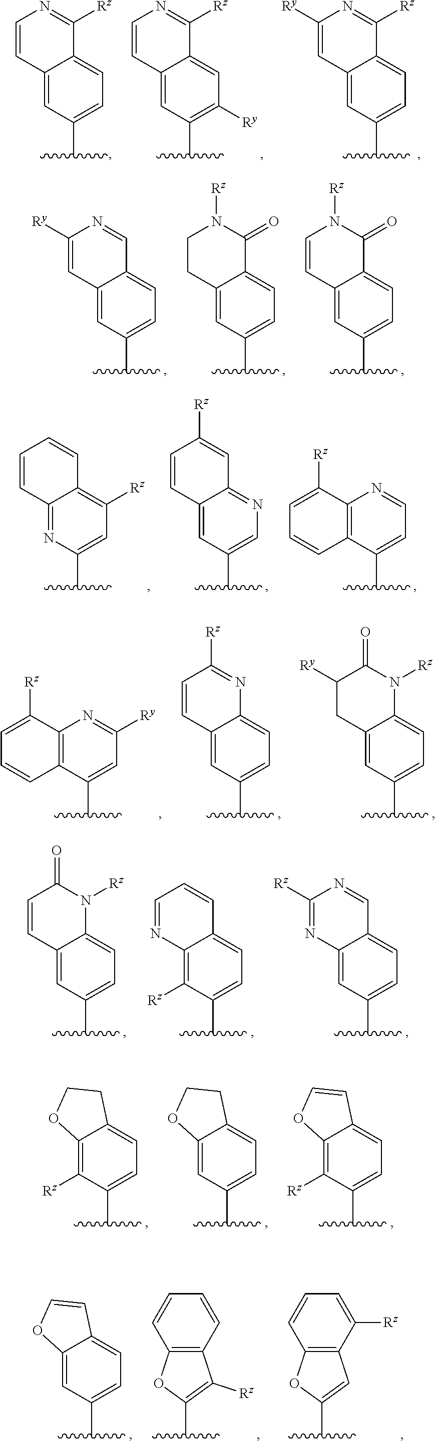

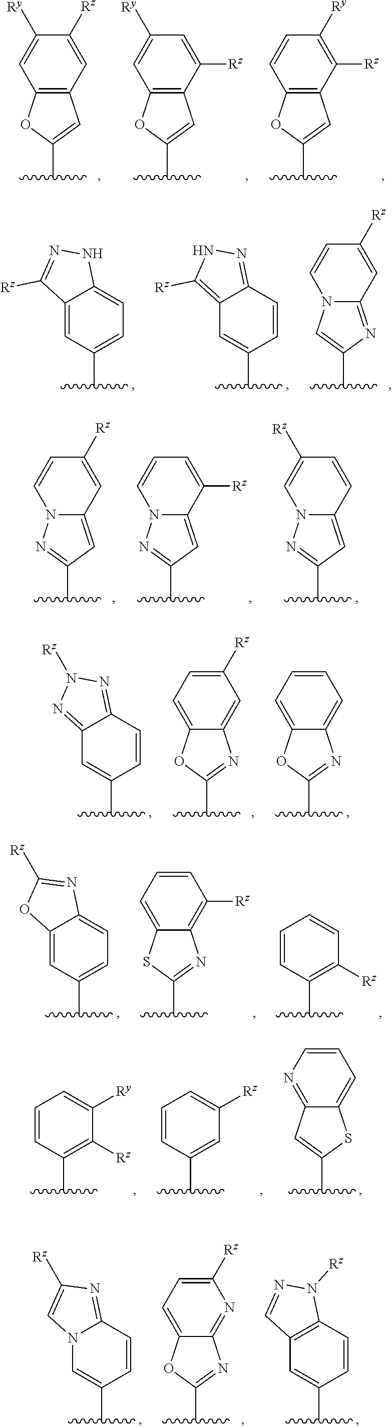

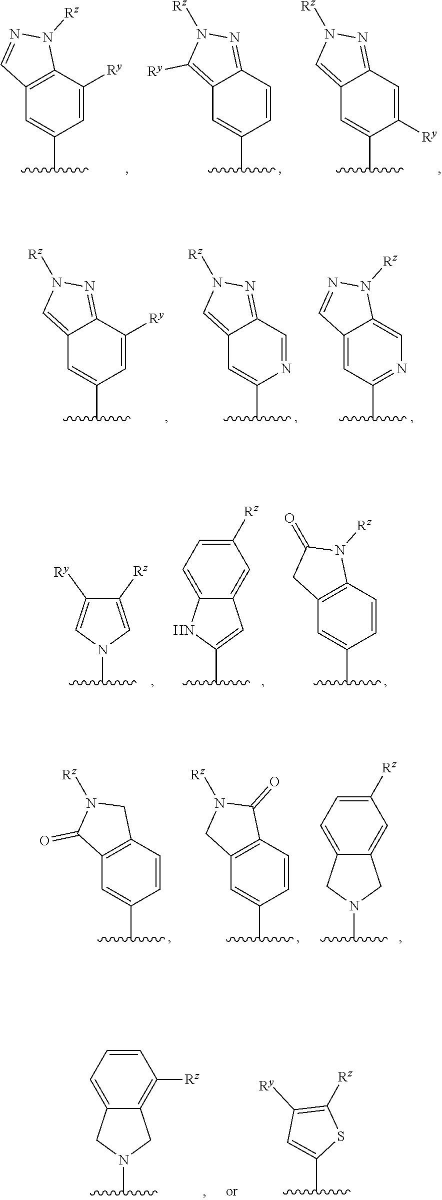

wherein R^{y} and R^{z} are each independently selected from H, halogen, cyano, cyclopropyl, cyclopropyl substituted with C₁-C₃ alkyl, cyclopropyl substituted with hydroxy substituted C₁-C₃ alkyl, C₁-C₃ alkyl, C₁-C₃ alkyl substituted with cyclopropyl, C₁-C₃ alkyl substituted with amino, C₁-C₃ alkyl substituted with C₁-C₃ alkoxy, C₁-C₃ alkyl substituted with hydroxy, C₁-C₃ alkyl substituted with one or more halogen, C₁-C₃ alkyl substituted with hydroxy and one or more halogen, C₁-C₃ alkyl substituted with cyano, C₁-C₃ alkyl substituted with —S(O)₂CH₃, C₂-C₄ alkenyl, C₂-C₄ alkynyl, C₁-C₃ alkoxy, C₁-C₃ alkoxy substituted with cyclopropyl, C₁-C₃ alkoxy substituted with one or more hydroxy, C₁-C₃ alkoxy substituted with one or more halogen, C₁-C₃ alkoxy substituted with hydroxy and one or more halogen, C₁-C₃ alkylamino, C₁-C₃ dialkylamino, pyridinyl, pyridinyl substituted with C₁-C₃ alkyl, 1H-1,2,4-triazol-3-yl, 1-methyl-1H-1,2,4-triazol-3-yl substituted with C₁-C₃ alkyl, wherein at least one of R^{y} and R^{z} is not H.

In an exemplary embodiment, the compound, or a salt or a hydrate or a solvate thereof, has a structure according to formula (I), wherein T and X are as described herein, and Z is

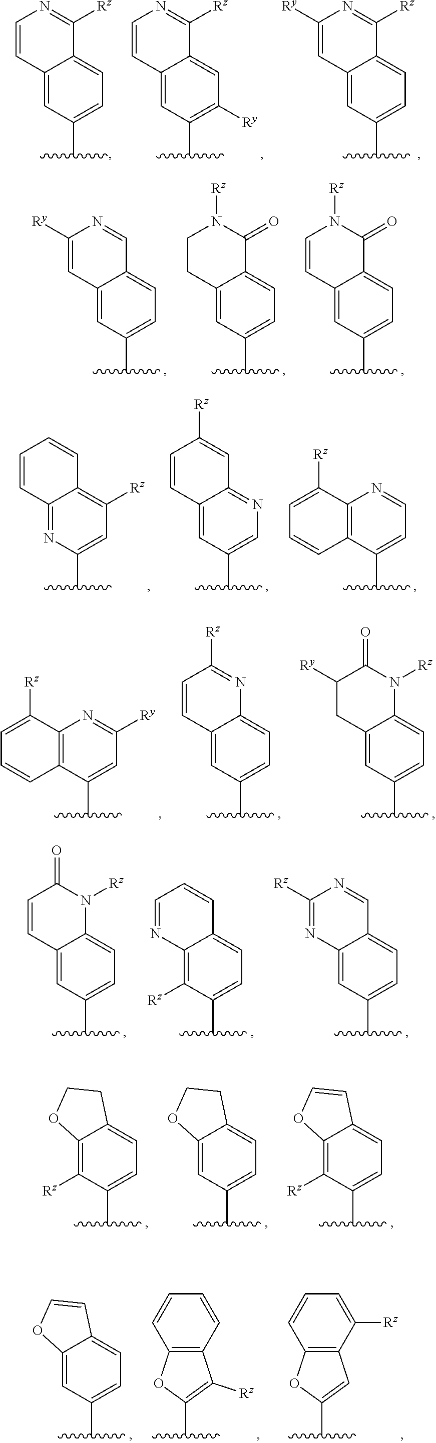

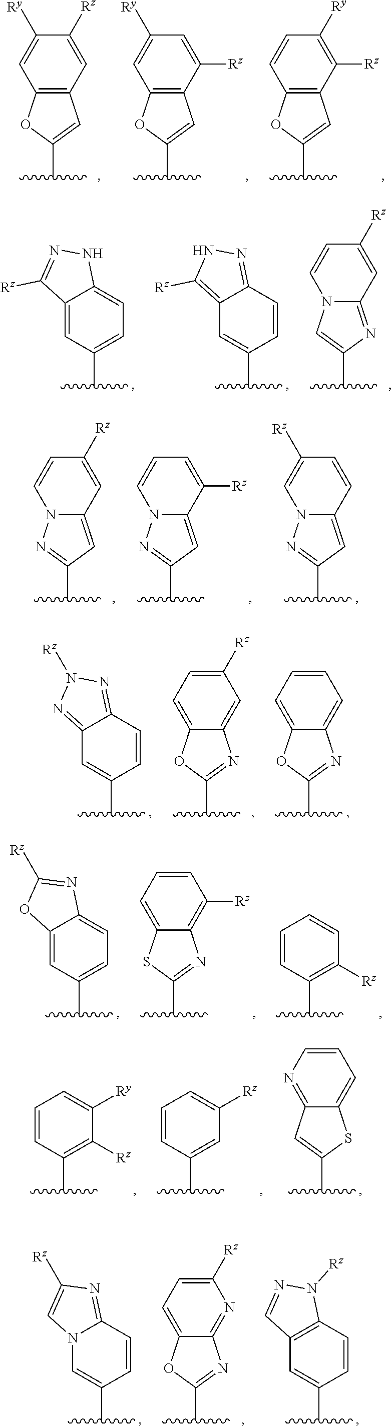

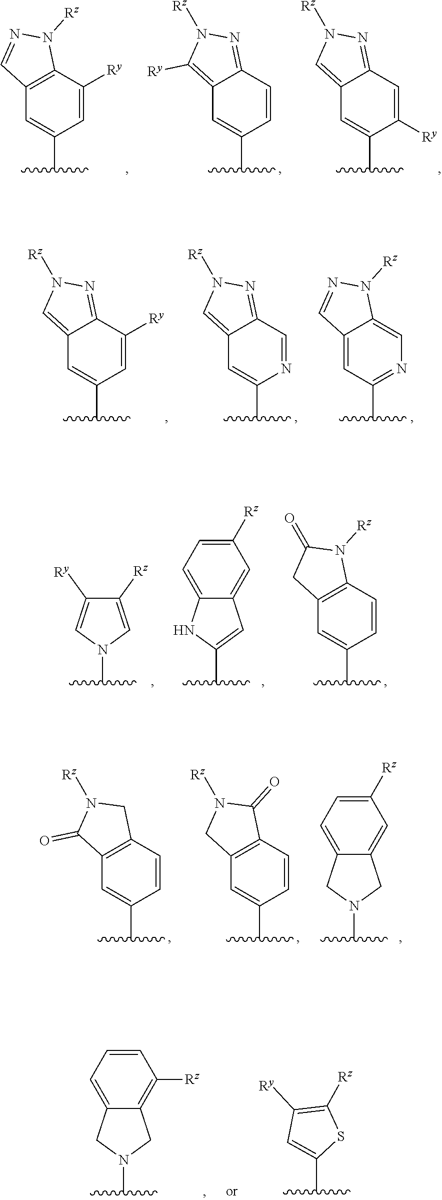

wherein R^{y} and R^{z} are each independently selected from H, F, C₁, cyano, (1S,2R)-2-(methoxymethyl)cyclopropyl, [(1R,2R)-2-(hydroxymethyl)cyclopropyl, (1S,2S)-2-(hydroxymethyl)cyclopropyl, (1R,2R)-2-methylcyclopropyl, cyclopropyl, 2-hydroxypropyl, methoxy, ethoxy, 2-hydroxyethoxy, methyl, difluoromethyl, trifluoromethyl, ethyl, 2-methoxyethyl, 2-aminoethyl, 2-hydroxyethyl, 2-fluoroethyl, 1,1-difluoroethyl, 2,2-difluoroethyl, 2,2,2-trifluoroethyl, propyl, isopropyl, 2-propynyl, methylamino, dimethylamino, cyanomethyl, 2-mesylethyl, wherein at least one of R^{y} and R^{z} is not H.

In an exemplary embodiment, the compound, or a salt or a hydrate or a solvate thereof, has a structure according to formula (I), wherein T and X are as described herein, and Z is [(1S,2R)-2-(methoxymethyl)cyclopropyl]-6-isoquinolyl, (2-methoxyethyl)-6-isoquinolyl, [(1R,2R)-2-(hydroxymethyl)cyclopropyl]-6-isoquinolyl, [(1S,2S)-2-(hydroxymethyl) cyclopropyl]-6-isoquinolyl, (1R,2R)-2-methylcyclopropyl]-6-isoquinolyl, (2-hydroxypropyl)-6-isoquinolyl, 1-methoxy-6-isoquinolyl, (2-hydroxyethoxy)-6-isoquinolyl, 1-methyl-6-isoquinolyl, 1-(methylamino)-6-isoquinolyl, 7-fluoro-1-methoxy-6-isoquinolyl, 2-methyl-1-oxo-3,4-dihydro-6-isoquinolyl, 1,3-dimethyl-6-isoquinolyl, 1-ethyl-6-isoquinolyl, 2-methyl-1-oxo-6-isoquinolyl, 5-fluoro-1-methoxy-6-isoquinolyl, 1-ethoxy-6-isoquinolyl, 1-(dimethylamino)-6-isoquinolyl, 3-methyl-6-isoquinolyl, 3-fluoro-6-isoquinolyl, 6-isoquinolyl, 4-methyl-2-quinolyl, 7-fluoro-3-quinolyl, 8-methyl-3-quinolyl, 8-methoxy-3-quinolyl, 7-chloro-3-quinolyl, 8-chloro-3-quinolyl, 3-quinolyl, 7-methyl-3-quinolyl, 8-methoxy-4-quinolyl, 8-methoxy-2-methyl-4-quinolyl, 2-ethoxy-6-quinolyl, 1-methyl-3-methyl-2-oxo-3,4-dihydro-6-quinolyl, 3-methyl-6-quinolyl, 2-methoxy-6-quinolyl, 2-methyl-6-quinolyl, 1-methyl-2-oxo-3,4-dihydro-6-quinolyl, 1-methyl-2-oxo-6-quinolyl, 6-quinolyl, 8-fluoro-7-quinolyl, 7-quinolyl, 2-methyl-7-quinazolinyl, 7-fluoro-2,3-dihydro-1-benzofuran-6-yl, 2,3-dihydro-1-benzofuran-6-yl, 3-chloro-1-benzofuran-2-yl, 4-methoxy-1-benzofuran-2-yl, 5,6-dimethoxy-1-benzofuran-2-yl, 4,6-dimethoxy-1-benzofuran-2-yl, 4-chloro-5-methoxy-1-benzofuran-2-yl, 5-methoxy-1-benzofuran-2-yl, 4-fluoro-1-benzofuran-2-yl, 1-benzofuran-2-yl, (2-aminoethyl)-1H-indazol-5-yl, (2-propynyl)-1H-indazol-5-yl, (2-propynyl)-2H-indazol-5-yl, 6-methoxy-1,3a-diaza-2-indenyl, 5-methoxy-1,7a-diaza-2-indenyl, 6-methoxy-1,7a-diaza-2-indenyl, 4-chloro-1,7a-diaza-2-indenyl, 6-methyl-1,7a-diaza-2-indenyl, 2-methyl-2H-1,2,3-benzotriazol-5-yl, 5-(1,1-difluoroethyl)-1,3-benzoxazol-2-yl, 5-(2,2,2-trifluoroethyl)-1,3-benzoxazol-2-yl, 5-isopropyl-1,3-benzoxazol-2-yl, 4-methoxy-1,3-benzoxazol-2-yl, 5-ethyl-1,3-benzoxazol-2-yl, 4-fluoro-1,3-benzoxazol-2-yl, 5-methoxy-1,3-benzoxazol-2-yl, 1,3-benzoxazol-2-yl, 2-methyl-1,3-benzoxazol-6-yl, 3-benzothiazol-2-yl, 4-methoxy-1,3-benzothiazol-2-yl, o-fluorophenyl, 2-fluoro-3-methoxyphenyl, m-tolyl, 1-thia-4-aza-2-indenyl, 1,7a-diaza-2-indenyl, 2-methyl-1,3a-diaza-5-indenyl, 5-cyclopropyl-1-oxa-3,4-diaza-2-indenyl, 1-(2-hydroxyethyl)-1H-indazol-5-yl, 1-(2-mesylethyl)-1H-indazol-5-yl, 1-cyanomethyl-1H-indazol-5-yl, 1-(2,2,2-trifluoroethyl)-1H-indazol-5-yl, 1-methyl-1H-indazol-5-yl, 1-cyclopropyl-1H-indazol-5-yl, 1-ethyl-1H-indazol-5-yl, 7-chloro-1-methyl-1H-indazol-5-yl, 3-chloro-2-methyl-2H-indazol-5-yl, 2-methyl-6-methyl-2H-indazol-5-yl, 2-methyl-7-methyl-2H-indazol-5-yl, (2-fluoroethyl)-2H-indazol-5-yl, 2-methyl-3-methyl-2H-indazol-5-yl, 2-(2,2-difluoroethyl)-2H-indazol-5-yl, 2-(2-hydroxyethyl)-2H-indazol-5-yl, 2-propyl-2H-indazol-5-yl, 2-(difluoromethyl)-2H-indazol-5-yl, 7-chloro-2-methyl-2H-indazol-5-yl, 7-fluoro-2-methyl-2H-indazol-5-yl, 2-ethyl-2H-indazol-5-yl, 2-methyl-2H-indazol-6-yl, 6-fluoro-2-methyl-2H-indazol-5-yl, 2-methyl-2H-indazol-5-yl, 2-methyl-2H-1,2,6-triazainden-5-yl, 1-methyl-1H-1,2,6-triazainden-5-yl, 2-ethyl-2H-1,2,6-triazainden-5-yl, 3,4-dimethyl-1-pyrrolyl, 5-cyano-2-indolyl, 1-methyl-2-oxo-5-indolinyl, 2-methyl-3-oxo-5-isoindolinyl, 2-methyl-1-oxo-5-isoindolinyl, 5-cyano-isoindolin-2-yl, 4-fluoro-2-isoindolinyl, or 4,5-dimethyl-2-thienyl.

Part d

In an exemplary embodiment, the compound, or a salt or a hydrate or a solvate thereof, has a structure according to formula (I), wherein T and X are as described herein, and Z is substituted or unsubstituted phenyl, substituted or unsubstituted pyridinyl, substituted or unsubstituted furan, substituted or unsubstituted thienyl, substituted or unsubstituted pyrimidinyl, and substituted or unsubstituted oxazolyl.

In an exemplary embodiment, the compound, or a salt or a hydrate or a solvate thereof, has a structure according to formula (I), wherein T and X are as described herein, and Z is substituted or unsubstituted isoquinolyl, substituted or unsubstituted quinolyl, substituted or unsubstituted indenyl, substituted or unsubstituted indazolyl, substituted or unsubstituted benzoxazolyl, substituted or unsubstituted pyrimidinyl, substituted or unsubstituted pyridinyl, substituted or unsubstituted benzofuranyl, or substituted or unsubstituted phenyl.

In an exemplary embodiment, the compound, or a salt or a hydrate or a solvate thereof, has a structure according to formula (I), wherein T and X are as described herein, and Z is isoquinolyl, quinolyl, indenyl, indazolyl, benzoxazolyl, pyrimidinyl, pyridinyl, benzofuranyl, or phenyl, each of which can be substituted with one or more members selected from the group consisting of halogen, C₂-C₄ alkenyl, unsubstituted C₁₋₃ alkyl, C₁₋₃ alkyl substituted with one or more halogen and/or hydroxy, unsubstituted cyclopropyl, unsubstituted C₁₋₃ alkoxy, and C₁₋₃ alkoxy substituted with one or more halogen.

In an exemplary embodiment, the compound, or a salt or a hydrate or a solvate thereof, has a structure according to formula (I), wherein T and X are as described herein, and Z is isoquinolyl, quinolyl, indenyl, indazolyl, benzoxazolyl, pyrimidinyl, pyridinyl, benzofuranyl, or phenyl, each of which can be substituted with one or more members selected from the group consisting of F, methyl, trifluoromethyl, (2-hydroxy)ethyl, (2-fluoro)ethyl, and cyclopropyl.

In an exemplary embodiment, the compound, or a salt or a hydrate or a solvate thereof, has a structure according to formula (I), wherein T and X are as described herein, and Z is

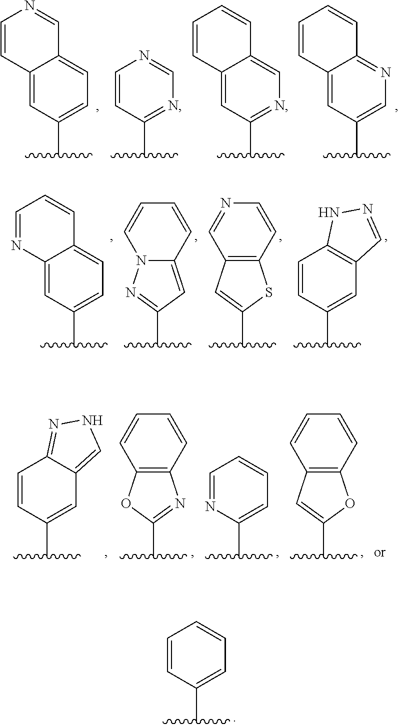

In an exemplary embodiment, the compound, or a salt or a hydrate or a solvate thereof, has a structure according to formula (I), wherein T and X are as described herein, and Z is

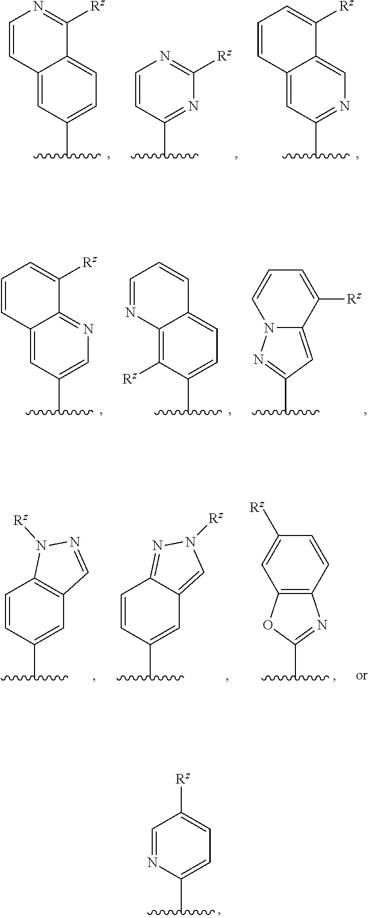

wherein R^{z} is halogen, C₂-C₄ alkenyl, unsubstituted C₁₋₃ alkyl, C₁₋₃ alkyl substituted with one or more halogen and/or hydroxy, cyclopropyl, unsubstituted C₁₋₃ alkoxy, or C₁₋₃ alkoxy substituted with one or more halogen.

In an exemplary embodiment, the compound, or a salt or a hydrate or a solvate thereof, has a structure according to formula (I), wherein T and X are as described herein, and Z is

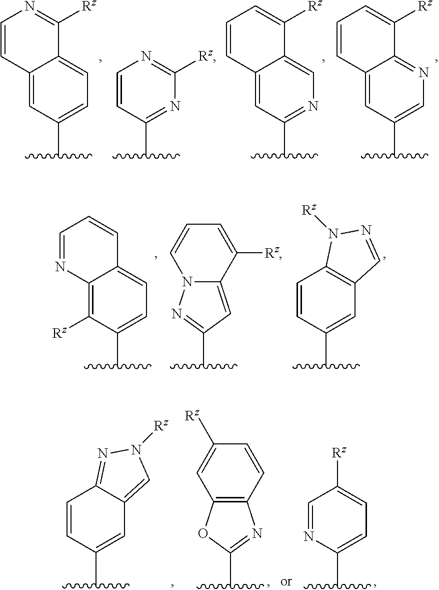

wherein R^{z} is F, methyl, trifluoromethyl, (2-hydroxy)ethyl, (2-fluoro)ethyl, or cyclopropyl.

In an exemplary embodiment, the compound, or a salt or a hydrate or a solvate thereof, has a structure according to formula (I), wherein T and X are as described herein, and Z is 1-methylisoquinolin-6-yl, 2-(trifluoromethyl)pyrimidin-4-yl, 2-methylpyrimidin-4-yl, 1-methyl-6-isoquinolyl, 1-(2-hydroxyethyl)-6-isoquinolyl, 3-isoquinolyl, 6-quinolyl, 8-fluoro-3-quinolyl, 8-fluoro-7-quinolyl, 4-methyl-1,7a-diaza-2-indenyl, 1-thia-5-aza-2-indenyl, 1-(2-fluoroethyl)-1H-indazol-5-yl, 3-quinolyl, 2-cyclopropyl-2H-indazol-5-yl, 6-fluoro-1,3-benzoxazol-2-yl, 5-fluoro-2-pyridyl, 1-benzofuran-2-yl, or phenyl.

Groups T & X:

In an exemplary embodiment, the compound, or a salt or a hydrate or a solvate thereof, has a structure according to formula (II):

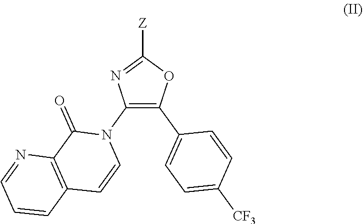

wherein Z is as defined herein.

In an exemplary embodiment, the compound, or a salt or a hydrate or a solvate thereof, has a structure according to formula (III):

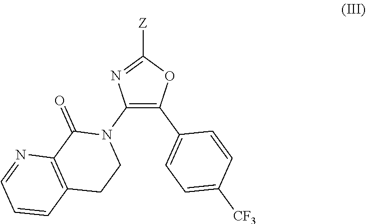

wherein Z is as defined herein.

In an exemplary embodiment, the compound, or a salt or a hydrate or a solvate thereof, has a structure according to formula (IV):

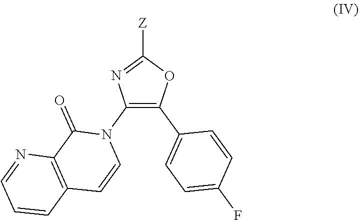

wherein Z is as defined herein.

In an exemplary embodiment, the compound, or a salt or a hydrate or a solvate thereof, has a structure according to formula (V):

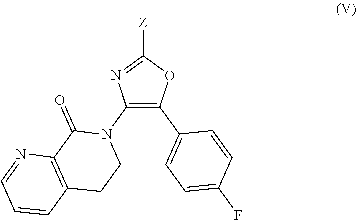

wherein Z is as defined herein.

In an exemplary embodiment, the compound, or a salt or a hydrate or a solvate thereof, has a structure according to formula (VI):

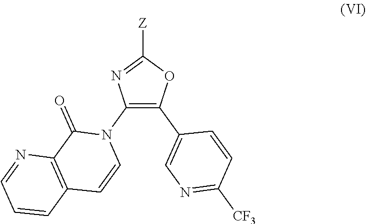

wherein Z is as defined herein.

In an exemplary embodiment, the compound, or a salt or a hydrate or a solvate thereof, has a structure according to formula (VII):

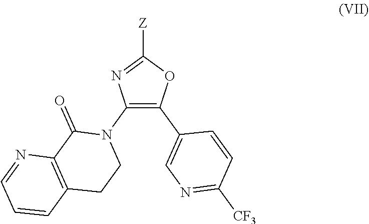

wherein Z is as defined herein.

In an exemplary embodiment, the compound, or a salt or a hydrate or a solvate thereof, has a structure according to formula (VIII):

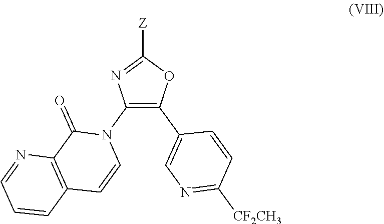

wherein Z is as defined herein.

In an exemplary embodiment, the compound, or a salt or a hydrate or a solvate thereof, has a structure according to formula (IX):

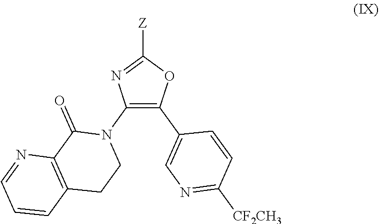

wherein Z is as defined herein.

### Groups X & Z:

In an exemplary embodiment, the compound, or a salt or a hydrate or a solvate thereof, has a structure according to formula (X):

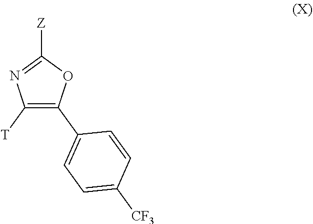

wherein T is as defined herein, and Z is 1-methylisoquinolin-6-yl, 2-(trifluoromethyl)pyrimidin-4-yl, 2-methylpyrimidin-4-yl, 1-methyl-6-isoquinolyl, 1-(2-hydroxyethyl)-6-isoquinolyl, 3-isoquinolyl, 6-quinolyl, 8-fluoro-3-quinolyl, 8-fluoro-7-quinolyl, 4-methyl-1,7a-diaza-2-indenyl, 1-thia-5-aza-2-indenyl, 1-(2-fluoroethyl)-1H-indazol-5-yl, 3-quinolyl, 2-cyclopropyl-2H-indazol-5-yl, 6-fluoro-1,3-benzoxazol-2-yl, 5-fluoro-2-pyridyl, 1-benzofuran-2-yl, or phenyl.

In an exemplary embodiment, the compound, or a salt or a hydrate or a solvate thereof, has a structure according to formula (XI):

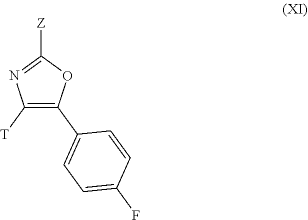

wherein T is as defined herein, and Z is 1-methylisoquinolin-6-yl, 2-(trifluoromethyl)pyrimidin-4-yl, 2-methylpyrimidin-4-yl, 1-methyl-6-isoquinolyl, 1-(2-hydroxyethyl)-6-isoquinolyl, 3-isoquinolyl, 6-quinolyl, 8-fluoro-3-quinolyl, 8-fluoro-7-quinolyl, 4-methyl-1,7a-diaza-2-indenyl, 1-thia-5-aza-2-indenyl, 1-(2-fluoroethyl)-1H-indazol-5-yl, 3-quinolyl, 2-cyclopropyl-2H-indazol-5-yl, 6-fluoro-1,3-benzoxazol-2-yl, 5-fluoro-2-pyridyl, 1-benzofuran-2-yl, or phenyl.

In an exemplary embodiment, the compound, or a salt or a hydrate or a solvate thereof, has a structure according to formula (XII):

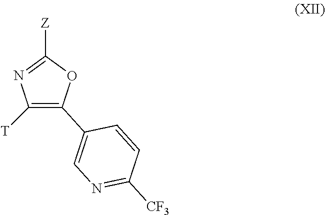

wherein T is as defined herein, and Z is 1-methylisoquinolin-6-yl, 2-(trifluoromethyl)pyrimidin-4-yl, 2-methylpyrimidin-4-yl, 1-methyl-6-isoquinolyl, 1-(2-hydroxyethyl)-6-isoquinolyl, 3-isoquinolyl, 6-quinolyl, 8-fluoro-3-quinolyl, 8-fluoro-7-quinolyl, 4-methyl-1,7a-diaza-2-indenyl, 1-thia-5-aza-2-indenyl, 1-(2-fluoroethyl)-1H-indazol-5-yl, 3-quinolyl, 2-cyclopropyl-2H-indazol-5-yl, 6-fluoro-1,3-benzoxazol-2-yl, 5-fluoro-2-pyridyl, 1-benzofuran-2-yl, or phenyl.

In an exemplary embodiment, the compound, or a salt or a hydrate or a solvate thereof, has a structure according to formula (XIII):

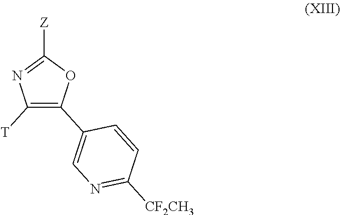

wherein T is as defined herein, and Z is 1-methylisoquinolin-6-yl, 2-(trifluoromethyl)pyrimidin-4-yl, 2-methylpyrimidin-4-yl, 1-methyl-6-isoquinolyl, 1-(2-hydroxyethyl)-6-isoquinolyl, 3-isoquinolyl, 6-quinolyl, 8-fluoro-3-quinolyl, 8-fluoro-7-quinolyl, 4-methyl-1,7a-diaza-2-indenyl, 1-thia-5-aza-2-indenyl, 1-(2-fluoroethyl)-1H-indazol-5-yl, 3-quinolyl, 2-cyclopropyl-2H-indazol-5-yl, 6-fluoro-1,3-benzoxazol-2-yl, 5-fluoro-2-pyridyl, 1-benzofuran-2-yl, or phenyl.

Groups T & Z:

In an exemplary embodiment, the compound, or a salt or a hydrate or a solvate thereof, has a structure according to formula (XIV):

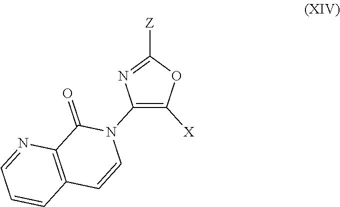

wherein X is as defined herein, and Z is 1-methylisoquinolin-6-yl, 2-(trifluoromethyl)pyrimidin-4-yl, 2-methylpyrimidin-4-yl, 1-methyl-6-isoquinolyl, 1-(2-hydroxyethyl)-6-isoquinolyl, 3-isoquinolyl, 6-quinolyl, 8-fluoro-3-quinolyl, 8-fluoro-7-quinolyl, 4-methyl-1,7a-diaza-2-indenyl, 1-thia-5-aza-2-indenyl, 1-(2-fluoroethyl)-1H-indazol-5-yl, 3-quinolyl, 2-cyclopropyl-2H-indazol-5-yl, 6-fluoro-1,3-benzoxazol-2-yl, 5-fluoro-2-pyridyl, 1-benzofuran-2-yl, or phenyl.

In an exemplary embodiment, the compound, or a salt or a hydrate or a solvate thereof, has a structure according to formula (XV):

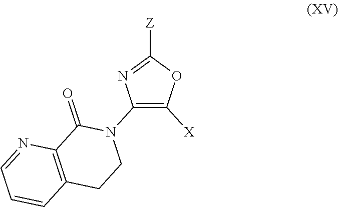

wherein X is as defined herein, and Z is 1-methylisoquinolin-6-yl, 2-(trifluoromethyl) pyrimidin-4-yl, 2-methylpyrimidin-4-yl, 1-methyl-6-isoquinolyl, 1-(2-hydroxyethyl)-6-isoquinolyl, 3-isoquinolyl, 6-quinolyl, 8-fluoro-3-quinolyl, 8-fluoro-7-quinolyl, 4-methyl-1,7a-diaza-2-indenyl, 1-thia-5-aza-2-indenyl, 1-(2-fluoroethyl)-1H-indazol-5-yl, 3-quinolyl, 2-cyclopropyl-2H-indazol-5-yl, 6-fluoro-1,3-benzoxazol-2-yl, 5-fluoro-2-pyridyl, 1-benzofuran-2-yl, or phenyl.

In an exemplary embodiment, the compound, or a salt or a hydrate or a solvate thereof, is 7-{5-[6-(1,1-difluoroethyl)-3-pyridyl]-2-(1-methyl-6-isoquinolyl)-1,3-oxazol-4-yl}-1,7-diaza-8(7H)-naphthalenone, 7-{2-[1-(2-hydroxyethyl)-6-isoquinolyl]-5-[6-(trifluoromethyl)-3-pyridyl]-1,3-oxazol-4-yl}-1,7-diaza-8(7H)-naphthalenone, 7-{2-[1-(2-hydroxyethyl)-6-isoquinolyl]-5-[p-(trifluoromethyl)phenyl]-1,3-oxazol-4-yl}-1,7-diaza-8(7H)-naphthalenone, 7-[5-(p-fluorophenyl)-2-(3-isoquinolyl)-1,3-oxazol-4-yl]-1,7-diaza-8(7H)-naphthalenone, 7-[5-(p-fluorophenyl)-2-(6-quinolyl)-1,3-oxazol-4-yl]-1,7-diaza-8(7H)-naphthalenone, 7-{2-(8-fluoro-3-quinolyl)-5-[p-(trifluoromethyl)phenyl]-1,3-oxazol-4-yl}-1,7-diaza-8(7H)-naphthalenone, 7-[5-(p-fluorophenyl)-2-(8-fluoro-7-quinolyl)-1,3-oxazol-4-yl]-1,7-diaza-8(7H)-naphthalenone, 7-{2-(4-methyl-1,7a-diaza-2-indenyl)-5-[p-(trifluoromethyl)phenyl]-1,3-oxazol-4-yl}-1,7-diaza-8(7H)-naphthalenone, 7-{2-(1-thia-5-aza-2-indenyl)-5-[p-(trifluoromethyl)phenyl]-1,3-oxazol-4-yl}-1,7-diaza-8(7H)-naphthalenone, 7-{2-[1-(2-fluoroethyl)-1H-indazol-5-yl]-5-[p-(trifluoromethyl)phenyl]-1,3-oxazol-4-yl}-1,7-diaza-8(7H)-naphthalenone, 7-{2-(3-quinolyl)-5-[p-(trifluoromethyl)phenyl]-1,3-oxazol-4-yl}-1,7-diaza-8(7H)-naphthalenone, 7-{2-(2-cyclopropyl-2H-indazol-5-yl)-5-[p-(trifluoromethyl)phenyl]-1,3-oxazol-4-yl}-1,7-diaza-8(7H)-naphthalenone, 7-{2-(6-fluoro-1,3-benzoxazol-2-yl)-5-[p-(trifluoromethyl)phenyl]-1,3-oxazol-4-yl}-1,7-diaza-8(7H)-naphthalenone, 7-{5-[p-(trifluoromethyl)phenyl]-2-[2-(trifluoromethyl)-4-pyrimidinyl]-1,3-oxazol-4-yl}-1,7-diaza-8(7H)-naphthalenone, 7-{2-(5-fluoro-2-pyridyl)-5-[p-(trifluoromethyl)phenyl]-1,3-oxazol-4-yl}-1,7-diaza-8(7H)-naphthalenone, 7-[2-(1-benzofuran-2-yl)-5-(p-fluorophenyl)-1,3-oxazol-4-yl]-1,7-diaza-8(7H)-naphthalenone, or 7-{2-phenyl-5-[p-(trifluoromethyl)phenyl]-1,3-oxazol-4-yl}-1,7-diaza-8(7H)-naphthalenone. In an exemplary embodiment, the compound, or a salt or a hydrate or a solvate thereof, is 7-(2-(2-methylpyrimidin-4-yl)-5-(4-(trifluoromethyl)phenyl)oxazol-4-yl)-6,7-dihydro-1,7-naphthyridin-8(5H)-one. In an exemplary embodiment, the compound, or a salt or a hydrate or a solvate thereof, is 7-(5-(4-(trifluoromethyl)phenyl)-2-(2-(trifluoromethyl)pyrimidin-4-yl)oxazol-4-yl)-1,7-naphthyridin-8(7H)-one. In an exemplary embodiment, the compound, or a salt or a hydrate or a solvate thereof, is 7-(2-(1-methylisoquinolin-6-yl)-5-(4-(trifluoromethyl)phenyl)oxazol-4-yl)-1,7-naphthyridin-8(7H)-one.

In an exemplary embodiment, the salt of a compound in this section is a pharmaceutically acceptable salt. In an exemplary embodiment, the salt of a compound described herein is a pharmaceutically acceptable salt. In an exemplary embodiment, the salt of a compound of the invention is a pharmaceutically acceptable salt.

In an exemplary embodiment, the invention provides a compound described herein, or a salt, hydrate or solvate thereof, or a combination thereof. In an exemplary embodiment, the invention provides a compound described herein, or a salt, hydrate or solvate thereof. In an exemplary embodiment, the invention provides a compound described herein, or a salt thereof. In an exemplary embodiment, the salt is a pharmaceutically acceptable salt. In an exemplary embodiment, the invention provides a compound described herein, or a hydrate thereof. In an exemplary embodiment, the invention provides a compound described herein, or a solvate thereof. In an exemplary embodiment, the invention provides a salt of a compound described herein. In an exemplary embodiment, the invention provides a pharmaceutically acceptable salt of a compound described herein. In an exemplary embodiment, the invention provides a hydrate of a compound described herein. In an exemplary embodiment, the invention provides a solvate of a compound described herein. In an exemplary embodiment, the invention provides any of the compounds disclosed herein where one or more atoms of the compounds disclosed are replaced with [2H], [3H], [11C], [18F], or [13N]. The compounds disclosed above and throughout bind to 4-8 amino acid residues of amino acid residues 86-99 of SEQ ID NO: 9.

### Embodiments of Formula (XVI)

In addition to the formula disclosed above, the present disclosure also provides compounds of formula (XVI).

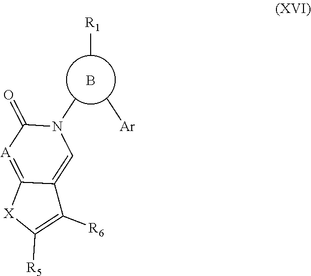

wherein:
R₁, R₅, and R₆ are each independently selected from aryl, substituted aryl, heteroaryl, substituted heteroaryl, alkyl, substituted alkyl, —CH₂R₂₀, —CHMeR₂₀, —CH(OH)R₂₀, cycloalkyl, substituted cycloalkyl, heterocycle, substituted heterocycle, alkenyl, alkynyl, acyl, nitro, halo, amino, substituted amine, ether, thioether, and H;
Ar is selected from aryl, substituted aryl, heteroaryl, and substituted heteroaryl;
A is selected from N, CH, C(halo);
X is selected from O and N—R₂,
R₂ and R₂₀ are each independently selected from aryl, substituted aryl, heteroaryl, substituted heteroaryl, alkyl, substituted alkyl, cycloalkyl, substituted cycloalkyl, heterocycle, substituted heterocycle, alkenyl, alkynyl, acyl, and H; and
Ring B is a 5-membered heteroaryl ring.

### Embodiments where X is NR₂

In some embodiments, X is NR₂, which is also referred to as “N—R₂” herein. In some of such cases, A is N. In other cases, A is C(halo), such as CF, CCl, or CBr. In other cases, A can be CH. Additionally, in some embodiments R₅ is H, R₆ is H, or both R₅ and R₆ are H. In some embodiments, Ring B is oxazole, wherein the structure and atom numbering of oxazole is shown below.

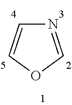

In some cases, Ring B is oxazole and R₁ is attached to the oxazole at C², Ar is attached to Ring B at C⁵, and the oxazole is attached to the remainder of the molecule at C⁴. This bonding arrangement is shown in the drawing below, and is also referred to herein as an embodiment wherein Ring B has Structure (M).

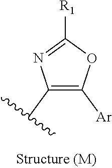

Thus, in some embodiments of formula (XVI), X is NR₂, A is N, R₅ and R₆ are both H, and Ring B has structure (M).

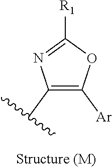

In some embodiments, R₁ is selected from the group consisting of alkyl, substituted alkyl, aryl, substituted aryl, heteroaryl, substituted heteroaryl, cycloalkyl, substituted cycloalkyl, heterocycle, and substituted heterocycle. In some cases, R₁ is a substituted alkyl, such as —CH₂R₂₀, —CHMeR₂₀, or —CH(OH)R₂₀. In some embodiments, R₂₀ is H, alkyl, or substituted alkyl.

### Embodiments where X is O

In other embodiments of formula (XVI), X is O. In some of such cases, A is N. In other cases, A is C(halo), such as CF, CCl, or CBr. In other cases, A can be CH. Additionally, in some embodiments R₅ is H, R₆ is H, or both R₅ and R₆ are H. In some embodiments, Ring B is oxazole, such as an oxazole with the bonding arrangement is shown in the drawing below:

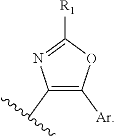

Thus, in some embodiments of formula (XVI), X is O, A is N, R₅ and R₆ are both H, and Ring B is

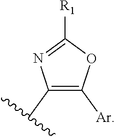

In some embodiments, R₁ is selected from the group consisting of alkyl, substituted alkyl, aryl, substituted aryl, heteroaryl, substituted heteroaryl, cycloalkyl, substituted cycloalkyl, heterocycle, and substituted heterocycle. In some cases, R₁ is a substituted alkyl, such as —CH₂R₂₀, —CHMeR₂₀, or —CH(OH)R₂₀. In some embodiments, R₂₀ is H, alkyl, or substituted alkyl. In some embodiments, R₁ is aryl, substituted aryl, heteroaryl, or substituted heteroaryl.

### Embodiments of Formula (XVII)

In some embodiments of formula (XVI), the compound has formula (XVII), as shown below. Formula (XVII) represents embodiments of formula (XVI) wherein Ring B is oxazole wherein R₁ is attached to the oxazole at C², Ar is attached to Ring B at C⁵, and the oxazole is attached to the remainder of the molecule at C⁴.

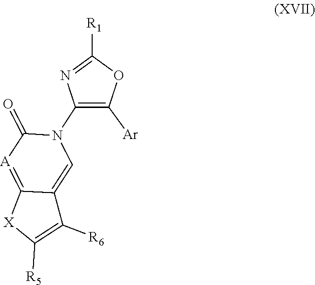

The embodiments of formula (XVII) can have each of the variations that are described above regarding formula (XVII). For example, A can be N or C(halo), such as CF. Additionally, X can be O or NR₂. One or both of R₅ and R₆ can be H.

In some embodiments, R₁ is selected from the group consisting of alkyl, substituted alkyl, aryl, substituted aryl, heteroaryl, substituted heteroaryl, cycloalkyl, substituted cycloalkyl, heterocycle, and substituted heterocycle. In some cases, R₁ is a substituted alkyl, such as —CH₂R₂₀, —CHMeR₂₀, or —CH(OH)R₂₀. In some embodiments, R₂₀ is H, alkyl, or substituted alkyl. In some embodiments, R₁ is aryl, substituted aryl, heteroaryl, or substituted heteroaryl.

### Embodiments of Formula (XIII)

In some embodiments of formula (XII), the compound can have formula (XIII). Formula (XIII) is a compound of formula (XII) wherein the Ar group is a phenyl ring with meta substituents of R₇ and R₉ along with a para substituent at R₈. Such R₇ through R₉ groups are selected from halogen, H, alkyl, substituted alkyl, alkoxy, substituted alkoxyl. Thus, in some cases each of such groups are H and the phenyl ring is unsubstituted.

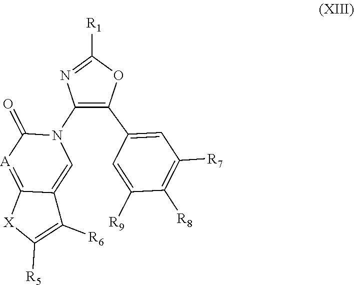

In some cases, the compound has formula (XIII)(a), (XIII)(b), (XIII)(c), (XIII)(d), (XIII)(e), or (XIII)(f):

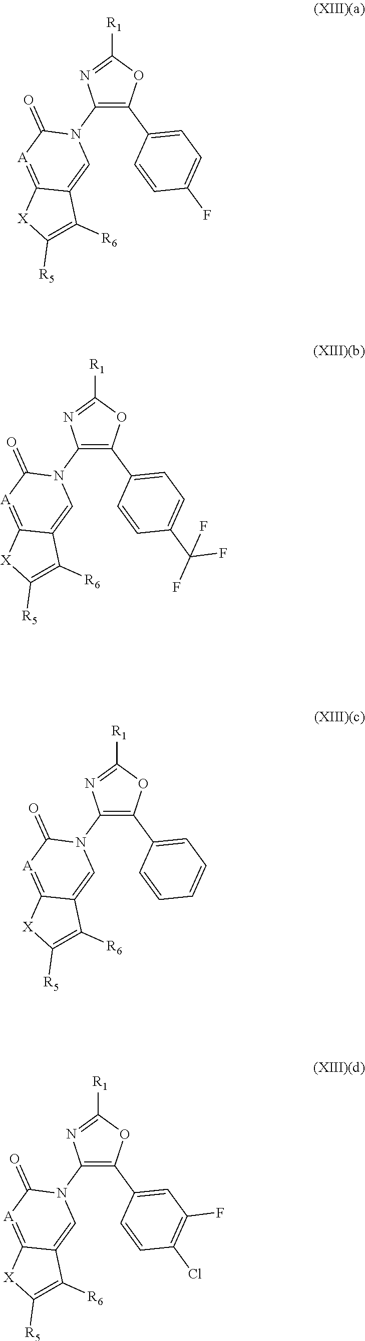

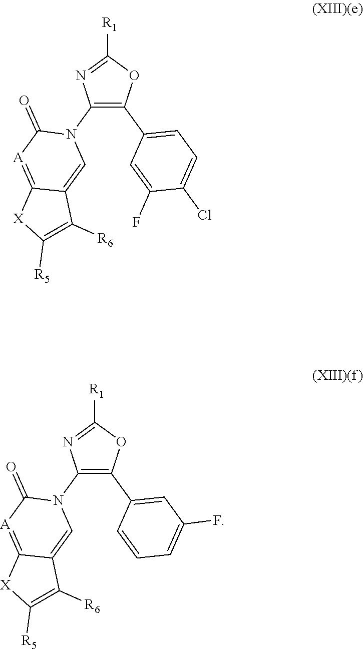

In embodiments of formula (XIII), in some cases A is N. In some cases, X is NR₂. In some case, X is O In some cases, both R₅ and R₆ are H. In some cases, Ring B has Structure (M), as discussed above.

In some embodiments, R₁ is selected from the group consisting of alkyl, substituted alkyl, aryl, substituted aryl, heteroaryl, substituted heteroaryl, cycloalkyl, substituted cycloalkyl, heterocycle, and substituted heterocycle. In some cases, R₁ is a substituted alkyl, such as —CH₂R₂₀, —CHMeR₂₀, or —CH(OH)R₂₀. In some embodiments, R₂₀ is H, alkyl, or substituted alkyl. In some embodiments, R₁ is aryl, substituted aryl, heteroaryl, or substituted heteroaryl.

### Embodiments of Formulas (XIV)

In some embodiments of formula (XII), the compound has formula (XIV)(a) or (XIV)(b):

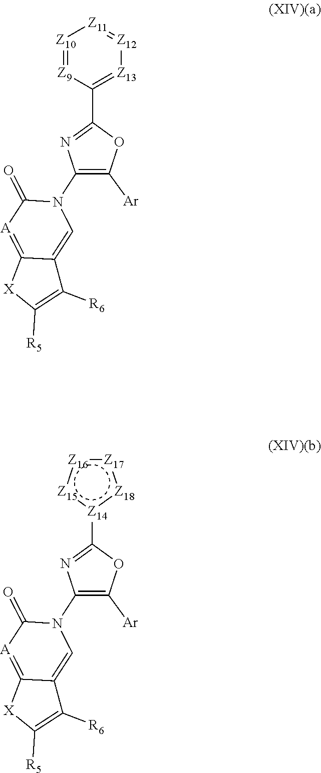

wherein:
Z₉-Z₁₃ are each independently selected from N and CR_{Z};
Z₁₄-Z₁₈ are each independently selected from N, O, S, and CR_{Z}; and
each R_{Z} is independently selected from the group consisting of H, alkyl, substituted alkyl, cycloalkyl, substituted alkyl, heterocycloalkyl, substituted heterocycloalkyl.

In some embodiments of formula (XIV)(a), one or more of Z₁₀-Z₁₂ are CR_{Z} and each R_{Z} is a non-hydrogen group.

The embodiments of formulas (XIV)(a) and (XIV)(b) can have each of the variations that are described above regarding formula (XVI). For example, A can be N or C(halo), such as CF. Additionally, X can be O or NR₂. One or both of R₅ and R₆ can be H.

In an exemplary embodiment, the invention provides any of the compound disclosed herein where one or more atoms of the compounds disclosed are replaced with [2H], [3H], [11C], [18F], or [13N]. The compounds disclosed above and throughout bind to 4-8 amino acid residues of amino acid residues 86-99 of SEQ ID NO: 9.

The compounds disclosed above form pi-pi interactions with each other thereby stacking with each other and with stacked proteins. In some case, pi-pi interactions are achieved by having: a planar core comprised of one or two rings which are optionally heterocyclic and an accessible molecular conformation allowing three or more of the molecules to self-associated in a repeating, parallel-displaced stack along the stacked proteins, a distance between an atom within adjacent planar core is 3.3-3.5 Å, a distance between equivalent atoms on two adjacent molecules is 4.8 Å, an angle (θ) between a line defined by two equivalent atoms on adjacent molecules and a line perpendicular to planes of the planar core that is 44°, and wherein a minimal distance between any atom within the plane of the core and an equivalent atom within adjacent molecule bound to a stacked protein is 3.2-3.6 Å. The compounds bind to the same site on each stacked protein thereby forming stacked compounds that are able to interrupt the propagation of stacked proteins. Any compounds having the above features associated with pi-pi interactions will be able to form pi-pi interactions among themselves and with the stacked proteins that they bind. In some cases, the compounds of the present disclosure are uniquely suited to interact with stacked proteins associated with a neurodegenerative disease of the central nervous system (CNS) because the compounds exist as low molecular weight monomers which allow for higher passive cell permeability allowing for easier crossing of the blood brain barrier. The monomers then may form oligomers once bound to the stack proteins of the present disclosure. The oligomers are then able to detect the presence of stack proteins, prions, amyloid fibrils, and templated misfolded proteins. The oligomers are also able to interrupt the propagation of misfolded proteins. The oligomers are also able to impeded the propagation of the amyloid conformation of a protein by impeding the sequestration of the native cellular protein and its conversion to the propagating amyloid form.

### Methods of Detecting a Neurological Disease

The present disclosure provides methods for detecting a neurological disease the method comprising isolating brain tissue from its natural environment, contacting the brain tissue with a labeled molecule which binds to multiple sites of stacked proteins associated with neurodegenerative disease, determining the binding of the labeled molecule; and thereby determining a neurological disease associated with the brain tissue.

The present disclosure also provides methods for detecting a neurological disease the method comprising contacting the brain tissue with a labeled molecule which binds to multiple sites of stacked proteins associated with neurological disease, determining the binding of the labeled molecule; and thereby determining a neurological disease associated with the brain tissue.

The contacting may be any form of contacting that results in the stacked proteins being bound by the labeled molecule. In some embodiments, the contacting is performed by administering the labeled molecule to an individual having or predicted to have the neurological disease. The administering may be any form of administering that results in the stacked proteins being bound by molecules. In some embodiments, the administering is performed by administering the molecules to an individual having or predicted to have the neurological disease. In some embodiments, the administration is intravenous. In some embodiments, the contacting involves direct administration of the labeled molecule to the isolated brain tissue.

The natural environment may be any environment in which stacked proteins associated with a neurodegenerative disease are found. In some embodiments, the natural environment is brain tissue. In some embodiments, the brains tissue is mammalian brain tissue. In some embodiments, the brain tissue is human brain tissue.

Brain tissues of the present disclosure may be any brain tissue deemed useful. In some embodiments, the brain tissue has or is suspected to have a neurological disease. In some embodiments, the brain tissue is human brain tissue that has or is suspected to have a neurological disease. The neurological disease is any neurological disease that is associated with stacked proteins including, without limitation, transmissible spongiform encephalopathies such as Creutzfeldt-Jakob disease (CJD), multiple system atrophy (MSA), Alzheimer's disease, Parkinson's disease, Amyotrophic lateral sclerosis (ALS), Amyotrophic lateral sclerosis/Parkinsonism-dementia complex, anti-IgLON5-related tauopathy, Caribbean Parkinsonism, Chronic traumatic encephalopathy, Diffuse neurofibrillary tangles with calcification, Down syndrome, Familial British dementia, Familial Danish dementia, Niemann-Pick disease, type C, Non-Guamanian motor neuron disease with neurofibrillary tangles, Postencephalitic Parkinsonism, Primary age-related tauopathy, Progressive ataxia and palatal tremor, Tangle-only dementia, Familial frontotemporal dementia and Parkinsonism, Pick's disease, Argyrophilic grain disease, Corticobasal degeneration, Guadeloupean Parkinsonism, Globular glial tauopathy, Huntington's disease, Progressive supranuclear palsy, SLC9a-related Parkinsonism, Tau astrogliopathy, etc. In some embodiments, the condition is multiple systems atrophy (MSA). In some embodiments, the condition is Parkinson's disease. In some embodiments, the condition is Alzheimer's disease.

The stacked proteins of the present disclosure may be any stacked protein that is associated with a neurological disease. Stacked proteins that are associated with a neurological disease include, without limitation, prions associated with transmissible spongiform encephalopathies such as Creutzfeldt-Jakob disease (CJD), α-synuclein associated with multiple system atrophy (MSA) and Parkinsons disease, amyloid β associated with Alzheimer's and Parkinson's disease, tau associated with Alzheimer's and Parkinson's disease, etc. In some embodiments, the stacked proteins are α-synuclein. In some embodiments, the stacked proteins are amyloid β. In some embodiments, the stacked proteins are tau.

Detecting a neurological disease of the present disclosure involves contacting brain tissue with a labeled molecule. The labeled molecule may be any molecule that is both able to bind to multiple sites of stacked proteins of a neurodegenerative disease and has a detectable label. The detectable label may be any detectable label that is detectable using Positron emission tomography (PET). Detectable labels include, without limitation, [2H], [3H], [11C], [18F], [13N], etc. Detectable labels that are detectable using PET are known in the art and have been described by, for example, Sun et al (Acc Chem Res. 2015 Feb. 17; 48(2):286-94) which is specifically incorporated by reference herein.

In some embodiments, the labeled molecule is a molecule according to formula (I). In some embodiments, the labeled molecule is a molecule according to formula (II). In some embodiments, the labeled molecule is a molecule according to formula (III). In some embodiments, the labeled molecule is a molecule according to formula (IV). In some embodiments, the labeled molecule is a molecule according to formula (V). In some embodiments, the labeled molecule is a molecule according to formula (VI). In some embodiments, the labeled molecule is a molecule according to formula (VII). In some embodiments, the labeled molecule is a molecule according to formula (VIII). In some embodiments, the labeled molecule is a molecule according to formula (IX). In some embodiments, the labeled molecule is a molecule according to formula (X). In some embodiments, the labeled molecule is a molecule according to formula (XI). In some embodiments, the labeled molecule is a molecule according to formula (XII). In some embodiments, the labeled molecule is a molecule according to formula (XIII)(a). In some embodiments, the labeled molecule is a molecule according to formula (XIII)(b). In some embodiments, the labeled molecule is a molecule according to formula (XIII)(c). In some embodiments, the labeled molecule is a molecule according to formula (XIII)(d). In some embodiments, the labeled molecule is a molecule according to formula (XIII)(e). In some embodiments, the labeled molecule is a molecule according to formula (XIII)(f). In some embodiments, the labeled molecule is a molecule according to formula (XIV)(a). In some embodiments, the labeled molecule is a molecule according to formula (XIV)(b). In some embodiments, the labeled molecule is a molecule according to any one of compounds 1-821 in FIG. 6. In some embodiments, the labeled molecule is any of the compounds discussed above that is detectably labeled.

Determining the binding of the labeled molecule of present disclosure involves detecting the presence of the labeled molecule in the brain tissue. The determining may be any method that is able to visualize the presence of the labeled molecule in the brain tissue. The determining includes, without limitation, PET, autoradiography, imaging mass spectrometry, magnetic resonance imaging, etc. In some embodiments, the determining is performed using PET. In some embodiments, the determining is performed using imaging mass spectrometry. In some embodiments, the determining is performed using magnetic resonance imaging. In some embodiments, the determining is performed using autoradiography. When the brain tissue has stacked proteins associated with a neurological disease, the labeled molecule will be bound to the stacked proteins in the brain tissue thereby determining the neurological disease in the brain tissue. In some embodiment, when the brain tissue has stacked proteins associated with a neurological disease, the labeled molecule will co-localize with the stacked proteins in the brain tissue thereby determining the neurological disease in the brain tissue.

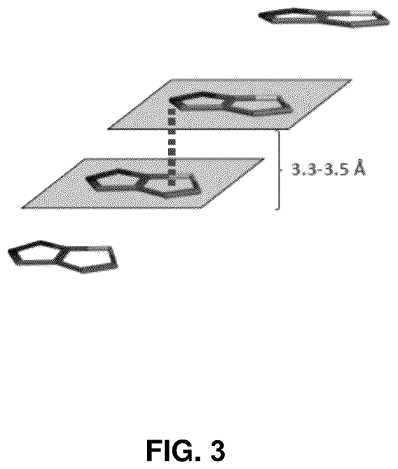

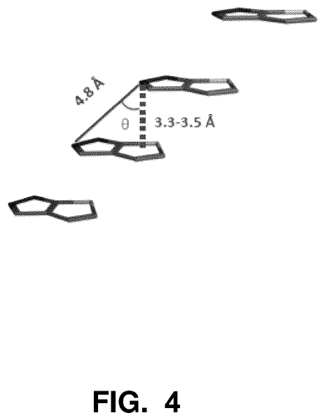

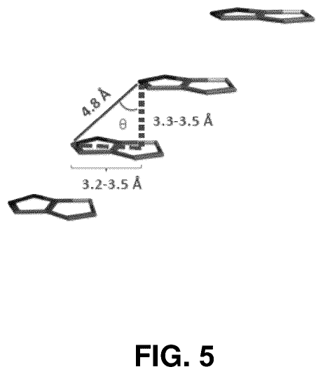

In some embodiment, the binding portion labeled molecule, i.e., the protein of the labeled molecule that binds to the stacked proteins, of the present disclosure is characterized by a planar core comprised of one or two rings which are optionally heterocyclic and an accessible molecular conformation allowing three or more of the molecules to self-associated in a repeating, parallel-displaced stack along the stacked proteins. In some embodiments, the binding portion of the labeled molecule is further characterized by a configuration shown in FIG. 3 where a distance between an atom within adjacent planar core is 3.3-3.5 Å. In some embodiments, the binding portion of the labeled molecule is further characterized by a configuration as shown in FIG. 4, wherein a distance between equivalent atoms on two adjacent molecules is 4.8 Å. In some embodiments, the binding portion of the labeled molecule is further characterized by a configuration as shown in FIG. 5, wherein an angle (θ) between a line defined by two equivalent atoms on adjacent molecules and a line perpendicular to planes of the planar core is 44°. In some embodiments, the binding portion of the labeled molecule is further characterized by a configuration as shown in FIG. 4, wherein a minimal distance between any atom within the plane of the core and an equivalent atom within adjacent molecule bound to a stacked protein is 3.2-3.6 Å.

In some embodiments, the binding portion of the labeled molecule is further characterized by substituents forming non-covalent interactions with the stacked proteins which interactions are selected from the group consisting of hydrogen bonds, Van der Waals contacts, pi-pi interactions, and chalcogen bonds. In some embodiments, the interactions are hydrogen bonds. In some embodiments, the interactions are Van der Waals contacts. In some embodiments, the interactions are hydrogen bonds. In some embodiments, the interactions are pi-pi interactions. In some embodiments, the interactions are hydrogen bonds. In some embodiments, the interactions are chalcogen bonds.

### Supramolecular Polymer Assembly Conjugates

The present disclosure provides supramolecular polymer assembly conjugates isolated from its natural environment, comprising stacked proteins of an α-synuclein prion associated with a neurodegenerative disease; and an α-synuclein prion inhibitor reversable bound to amyloid fibrils of the α-synuclein prion. In some embodiments, the reversible bond is a non-covalent bond.

The natural environment may be any environment in which stacked proteins of an α-synuclein prion associated with a neurodegenerative disease are found. In some embodiments, the natural environment is brain tissue. In some embodiments, the brain tissue is human brain tissue.

The α-synuclein prion inhibitor may be any α-synuclein prion inhibitor that inhibits α-synuclein from forming stacked proteins, e.g., prions. In some embodiments, the α-synuclein prion inhibitor has a structure according to formula (I). In some embodiments, the α-synuclein prion inhibitor has a structure according to formula (II). In some embodiments, the α-synuclein prion inhibitor has a structure according to formula (III). In some embodiments, the α-synuclein prion inhibitor has a structure according to formula (IV). In some embodiments, the α-synuclein prion inhibitor has a structure according to formula (V). In some embodiments, the α-synuclein prion inhibitor has a structure according to formula (VI). In some embodiments, the α-synuclein prion inhibitor has a structure according to formula (VII). In some embodiments, the α-synuclein prion inhibitor has a structure according to formula (VIII). In some embodiments, the α-synuclein prion inhibitor has a structure according to formula (IX). In some embodiments, the α-synuclein prion inhibitor has a structure according to formula (X). In some embodiments, the α-synuclein prion inhibitor has a structure according to formula (XI). In some embodiments, the α-synuclein prion inhibitor has a structure according to formula (XII). In some embodiments, the α-synuclein prion inhibitor has a structure according to formula (XIII)(a). In some embodiments, the α-synuclein prion inhibitor has a structure according to formula (XIII)(b). In some embodiments, the α-synuclein prion inhibitor has a structure according to formula (XIII)(c). In some embodiments, the α-synuclein prion inhibitor has a structure according to formula (XIII)(d). In some embodiments, the α-synuclein prion inhibitor has a structure according to formula (XIII)(e). In some embodiments, the α-synuclein prion inhibitor has a structure according to formula (XIII)(f). In some embodiments, the α-synuclein prion inhibitor has a structure according to formula (XIV)(a). In some embodiments, the α-synuclein prion inhibitor has a structure according to formula (XIV)(b). In some embodiments, the α-synuclein prion inhibitor has a structure according to any one of compounds 1-821 in FIG. 6. In some embodiments, the α-synuclein prion inhibitor is any of the compounds discussed above.

In some embodiment, the α-synuclein prion inhibitor of the present disclosure is characterized by a planar core comprised of one or two rings which are optionally heterocyclic and an accessible molecular conformation allowing three or more of the molecules to self-associated in a repeating, parallel-displaced stack along the stacked proteins. In some embodiments, the α-synuclein prion inhibitor is further characterized by a configuration shown in FIG. 3 where a distance between an atom within adjacent planar core is 3.3-3.5 Å. In some embodiments, α-synuclein prion inhibitor is further characterized by a configuration as shown in FIG. 4, wherein a distance between equivalent atoms on two adjacent molecules is 4.8 Å. In some embodiments, the α-synuclein prion inhibitor is further characterized by a configuration as shown in FIG. 5, wherein an angle (θ) between a line defined by two equivalent atoms on adjacent molecules and a line perpendicular to planes of the planar core is 44°. In some embodiments, α-synuclein prion inhibitor is further characterized by a configuration as shown in FIG. 4, wherein a minimal distance between any atom within the plane of the core and an equivalent atom within adjacent molecule bound to a stacked protein is 3.2-3.6 Å. In some embodiments, the α-synuclein prion inhibitor is further characterized by being uniquely suited to interact with the stacked proteins associated with a neurodegenerative disease of the central nervous system (CNS).

In some cases, the α-synuclein prion inhibitors of the present disclosure are uniquely suited to interact with stacked proteins associated with a neurodegenerative diseases because the α-synuclein prion inhibitors form pi-pi interactions with each other thereby stacking with each other and with stacked proteins. In some case, pi-pi interactions are achieved by having: a planar core comprised of one or two rings which are optionally heterocyclic and an accessible molecular conformation allowing three or more of the molecules to self-associated in a repeating, parallel-displaced stack along the stacked proteins, a distance between an atom within adjacent planar core is 3.3-3.5 Å, a distance between equivalent atoms on two adjacent molecules is 4.8 Å, an angle (θ) between a line defined by two equivalent atoms on adjacent molecules and a line perpendicular to planes of the planar core that is 44°, and wherein a minimal distance between any atom within the plane of the core and an equivalent atom within adjacent molecule bound to a stacked protein is 3.2-3.6 Å. In some cases, the α-synuclein prion inhibitors of the present disclosure are uniquely suited to interact with stacked proteins associated with a neurodegenerative disease of the central nervous system (CNS) because the α-synuclein prion inhibitors exist as low molecular weight monomers which allow for higher passive cell permeability allowing for easier crossing of the blood brain barrier. The monomers then may form oligomers once bound to the stack proteins of the present disclosure.

In some embodiments, the α-synuclein prion inhibitor is detectably labeled. Detectable labels include, without limitation, [2H], [3H], [11C], [18F], [13N], etc.

### Pharmaceutical Formulations

The herein-discussed compounds, α-synuclein prion inhibitors, labeled molecules, and molecules can be formulated using any convenient excipients, reagents and methods. Compositions are provided in formulation with a pharmaceutically acceptable excipient(s). A wide variety of pharmaceutically acceptable excipients are known in the art and need not be discussed in detail herein. Pharmaceutically acceptable excipients have been amply described in a variety of publications, including, for example, A. Gennaro (2000) “Remington: The Science and Practice of Pharmacy,” 20th edition, Lippincott, Williams, & Wilkins; Pharmaceutical Dosage Forms and Drug Delivery Systems (1999) H. C. Ansel et al., eds., 7^{th} ed., Lippincott, Williams, & Wilkins; and Handbook of Pharmaceutical Excipients (2000) A. H. Kibbe et al., eds., 3^{rd} ed. Amer. Pharmaceutical Assoc.

In an exemplary embodiment, the invention provides a pharmaceutical formulation comprising: a) the compound, or a pharmaceutically acceptable salt or a hydrate or a solvate thereof, described herein; and b) a pharmaceutically acceptable excipient. The pharmaceutical formulation can be administered by any suitable means, such as for example oral and/or parenteral depot. Oral administration in the form of a pill, capsule, elixir, syrup, lozenge, troche, or the like is particularly preferred. Dosage levels of the order of from about 5 mg to about 250 mg per kilogram of body weight per day and more preferably from about 25 mg to about 150 mg per kilogram of body weight per day, are useful in the treatment of the diseases described herein. Frequency of dosage may also vary depending on the compound used and the particular disease treated. However, for treatment of most disorders, a dosage regimen of 4 times daily or less is preferred. It will be understood, however, that the specific dose level for any particular patient will depend upon a variety of factors including the activity of the specific compound employed, the age, body weight, general health, sex, diet, time of administration, route of administration and rate of excretion, drug combination and the severity of the particular disease undergoing therapy. Preferred compounds of the invention will have desirable pharmacological properties that include, but are not limited to, oral bioavailability, low toxicity, low serum protein binding and desirable in vitro and in vivo half-lives. Penetration of the blood brain barrier for compounds used to treat CNS disorders is necessary, while low brain levels of compounds used to treat peripheral disorders are often preferred. In an exemplary embodiment, the invention provides an oral pharmaceutical formulation comprising: a) the compound, or a pharmaceutically acceptable salt or a hydrate or a solvate thereof, described herein; and b) a pharmaceutically acceptable excipient suitable for oral administration. In an exemplary embodiment, the invention provides a parenteral pharmaceutical formulation comprising: a) the compound, or a pharmaceutically acceptable salt or a hydrate or a solvate thereof, described herein; and b) a pharmaceutically acceptable excipient suitable for parenteral administration. In an exemplary embodiment, the invention provides an intravenous pharmaceutical formulation comprising: a) the compound, or a pharmaceutically acceptable salt or a hydrate or a solvate thereof, described herein; and b) a pharmaceutically acceptable excipient suitable for intravenous administration. In some embodiments, the pharmaceutical formulation is an injectable formulation. In some embodiments, the pharmaceutical formulation is an oral formulation. In some embodiments, the pharmaceutical formulation is a parenteral formulation.

The subject compounds may be administered in a unit dosage form and may be prepared by any methods well known in the art. Such methods include combining the subject compound with a pharmaceutically acceptable carrier or diluent which constitutes one or more accessory ingredients. A pharmaceutically acceptable carrier is selected on the basis of the chosen route of administration and standard pharmaceutical practice. Each carrier must be “pharmaceutically acceptable” in the sense of being compatible with the other ingredients of the formulation and not injurious to the subject. This carrier can be a solid or liquid and the type is generally chosen based on the type of administration being used.

The compounds of the invention may also be used in combination with additional therapeutic agents. In an exemplary embodiment, the invention provides a combination comprising: a) the compound, or a pharmaceutically acceptable salt or a hydrate or a solvate thereof, described herein; and b) at least one additional therapeutic agent. In an exemplary embodiment, the additional therapeutic agent is useful in treating a neurodegenerative disease.

Any drug delivery device or system that provides for the dosing regimen of the instant disclosure can be used. A wide variety of delivery devices and systems are known to those skilled in the art.

In some embodiments, compounds, α-synuclein prion inhibitors, labeled molecules, and molecules in the formulation are detectably labeled. Detectable labels include, without limitation, [2H], [3H], [11C], [18F], [13N], etc.

### Method of Interrupting Propagation of Stacked Proteins

The present disclosure provides methods for interrupting propagation of stacked proteins associated with a neurological disease, comprising contacting an environment populated with stacked proteins associated with a neurological disease with molecules which bind multiple sites of the stacked proteins; allowing the molecules to bind multiple sites of the stacked proteins; and thereby impeding propagation of the stacked proteins in the environment. In some embodiments, the method for interrupting propagation of stacked proteins associated with a neurological disease treats the neurological disease.

The environment may be any environment in which stacked proteins associated with a neurodegenerative disease including, without limitation, cell lysate, cell culture, mammalian brain tissue, etc. In some embodiments, the environment is mammalian brain tissue. In some embodiments, the mammalian brain tissue is human brain tissue. In some embodiments, the environment is a cell lysate. In some embodiments, the environment is a cell culture. In some embodiments, the cell culture is mammalian cell culture. In some embodiments, the cell culture contains neurons. In some embodiments, the brain or brain tissue is within an individual having or is suspected to have a neurological disease.

The neurological disease is any neurological disease that is associated with stacked proteins including, without limitation, transmissible spongiform encephalopathies such as Creutzfeldt-Jakob disease (CJD), multiple system atrophy (MSA), Alzheimer's disease, Parkinson's disease, Amyotrophic lateral sclerosis (ALS), Amyotrophic lateral sclerosis/Parkinsonism-dementia complex, anti-IgLON5-related tauopathy, Caribbean Parkinsonism, Chronic traumatic encephalopathy, Diffuse neurofibrillary tangles with calcification, Down syndrome, Familial British dementia, Familial Danish dementia, Niemann-Pick disease, type C, Non-Guamanian motor neuron disease with neurofibrillary tangles, Postencephalitic Parkinsonism, Primary age-related tauopathy, Progressive ataxia and palatal tremor, Tangle-only dementia, Familial frontotemporal dementia and Parkinsonism, Pick's disease, Argyrophilic grain disease, Corticobasal degeneration, Guadeloupean Parkinsonism, Globular glial tauopathy, Huntington's disease, Progressive supranuclear palsy, SLC9a-related Parkinsonism, Tau astrogliopathy, etc. In some embodiments, the neurological disease is multiple systems atrophy (MSA). In some embodiments, the neurological disease is Parkinson's disease. In some embodiments, the neurological disease is Alzheimer's disease.

The stacked proteins of the present disclosure may be any stacked protein that is associated with a neurological disease. Stacked proteins that are associated with a neurological disease include, without limitation, prions associated with transmissible spongiform encephalopathies such as Creutzfeldt-Jakob disease (CJD), α-synuclein associated with multiple system atrophy (MSA) and Parkinsons disease, amyloid β associated with Alzheimer's and Parkinson's disease, tau associated with Alzheimer's and Parkinson's disease, etc. In some embodiments, the stacked proteins are a-synuclein. In some embodiments, the stacked proteins are amyloid β. In some embodiments, the stacked proteins are tau.

The contacting may be any form of contacting that results in the stacked proteins being bound by molecules. In some embodiments, the contacting is performed by administering the molecules to an individual having or predicted to have the neurological disease. In some embodiments, the administration is through oral administration. In some embodiments the administration is through parenteral depot. In some embodiments, the administration is intravenous. In some embodiments, the contacting involves administration of an effective dose of the molecule to an individual having or is suspected to have a neurological disease.

An effective dose as used herein means a dose sufficient to alleviate symptoms associated with the neurological disease. The term effective dose also refers to the amount of an agent that is sufficient to effect beneficial or desired results. The effective dose will vary depending upon the subject and neurological disease being treated, the weight and age of the subject, the severity of the disease condition, the manner of administration and the like, which can readily be determined by one of ordinary skill in the art. The term also applies to a dose that will provide an image for detection by any one of the detection methods described herein. The specific dose will vary depending on the particular compound or molecule chosen, the dosing regimen to be followed, whether it is administered in combination with other compounds, timing of administration, the tissue to be imaged, and the physical delivery system in which it is carried. The effective dose is any dose that halts, reverses, or alleviates the neurological condition.

The halting, reversal or alleviation of the neurological condition may be determined by evaluating an individual having or suspecting to have a neurological disease before and after the contacting. The halting, reversal, or alleviation of the neurological condition may be determined by determining if the propagation of stacked proteins associated with a neurological disease is interrupted by, for example, using the detection methods disclosed above or herein.

The molecules may be any molecules that inhibit stacked proteins, e.g., prions, from propagating. In some embodiments, the molecule is a molecule according to formula (I). In some embodiments, the molecule is a molecule according to formula (II). In some embodiments, the molecule is a molecule according to formula (III). In some embodiments, the molecule is a molecule according to formula (IV). In some embodiments, the molecule is a molecule according to formula (V). In some embodiments, the molecule is a molecule according to formula (VI). In some embodiments, the molecule is a molecule according to formula (VII). In some embodiments, the molecule is a molecule according to formula (VIII). In some embodiments, the molecule is a molecule according to formula (IX). In some embodiments, the molecule is a molecule according to formula (X). In some embodiments, the molecule is a molecule according to formula (XI). In some embodiments, the molecule is a molecule according to formula (XII). In some embodiments, the molecule is a molecule according to formula (XIII)(a). In some embodiments, the molecule is a molecule according to formula (XIII)(b). In some embodiments, the molecule is a molecule according to formula (XIII)(c). In some embodiments, the molecule is a molecule according to formula (XIII)(d). In some embodiments, the molecule is a molecule according to formula (XIII)(e). In some embodiments, the molecule is a molecule according to formula (XIII)(f). In some embodiments, the molecule is a molecule according to formula (XIV)(a). In some embodiments, the molecule is a molecule according to formula (XIV)(b). In some embodiments, the molecule is a molecule according to any one of compounds 1-821 in FIG. 6. In some embodiments, the molecule is any of the compounds discussed above.

In some embodiment, the molecule of the present disclosure is characterized by a planar core comprised of one or two rings which are optionally heterocyclic and an accessible molecular conformation allowing three or more of the molecules to self-associated in a repeating, parallel-displaced stack along the stacked proteins. In some embodiments, the molecule is further characterized by a configuration shown in FIG. 3 where a distance between an atom within adjacent planar core is 3.3-3.5 Å. In some embodiments, molecule is further characterized by a configuration as shown in FIG. 4, wherein a distance between equivalent atoms on two adjacent molecules is 4.8 Å. In some embodiments, the molecule is further characterized by a configuration as shown in FIG. 5, wherein an angle (θ) between a line defined by two equivalent atoms on adjacent molecules and a line perpendicular to planes of the planar core is 44°. In some embodiments, molecule is further characterized by a configuration as shown in FIG. 4, wherein a minimal distance between any atom within the plane of the core and an equivalent atom within adjacent molecule bound to a stacked protein is 3.2-3.6 Å.

In some embodiments, the molecules which bind stacked proteins is further characterized by substituents forming non-covalent interactions with the stacked proteins which interactions are selected from the group consisting of hydrogen bonds, Van der Waals contacts, pi-pi interactions, and chalcogen bonds. In some embodiments, the interactions are hydrogen bonds. In some embodiments, the interactions are Van der Waals contacts. In some embodiments, the interactions are hydrogen bonds. In some embodiments, the interactions are pi-pi interactions. In some embodiments, the interactions are hydrogen bonds. In some embodiments, the interactions are chalcogen bonds.

In some cases, the molecules of the present disclosure are uniquely suited to interact with stacked proteins associated with neurological diseases because the molecules form pi-pi interactions with each other thereby stacking with each other and with stacked proteins. In some case, pi-pi interactions are achieved by having: a planar core comprised of one or two rings which are optionally heterocyclic and an accessible molecular conformation allowing three or more of the molecules to self-associated in a repeating, parallel-displaced stack along the stacked proteins, a distance between an atom within adjacent planar core is 3.3-3.5 Å, a distance between equivalent atoms on two adjacent molecules is 4.8 Å, an angle (θ) between a line defined by two equivalent atoms on adjacent molecules and a line perpendicular to planes of the planar core that is 44°, and wherein a minimal distance between any atom within the plane of the core and an equivalent atom within adjacent molecule bound to a stacked protein is 3.2-3.6 Å. The molecules bind to the same site on each stacked protein thereby forming stacked molecules that are able to interrupt the propagation of stacked proteins. In some cases, the molecules of the present disclosure are uniquely suited to interact with stacked proteins associated with a neurodegenerative disease of the central nervous system (CNS) because the molecules exists as low molecular weight monomers which allow for higher passive cell permeability allowing for easier crossing of the blood brain barrier. The monomers then may form oligomers once bound to the stack proteins of the present disclosure.

### Method of Impeding Progressive Templated Misfolding Proteins

The present disclosure provides herein methods for impeding progressive, templated misfolding of proteins associated with neurodegenerative disease, comprising administering molecules into a biological milieu containing both a propagating amyloid conformation of a protein and a native cellular form of the same protein; allowing for formation of a complex between a supramolecular polymer assembly of the molecules and a supramolecular assembly of the protein; and thereby impeding further sequestration of the native cellular protein and its conversion to the propagating amyloid form.

The biological milieu may be any biological milieu in which templated misfolding of proteins associated with a neurodegenerative disease including, without limitation, cell lysate, cell culture, mammalian brain tissue, etc. In some embodiments, the biological milieu is mammalian brain tissue. In some embodiments, the mammalian brain tissue is human brain tissue. In some embodiments, the biological milieu is a cell lysate. In some embodiments, the biological milieu is a cell culture. In some embodiments, the cell culture is mammalian cell culture. In some embodiments, the cell culture contains neurons.

The neurodegenerative disease is any neurodegenerative disease that is associated with stacked proteins including, without limitation, transmissible spongiform encephalopathies such as Creutzfeldt-Jakob disease (CJD), multiple system atrophy (MSA), Alzheimer's disease, Parkinson's disease, Amyotrophic lateral sclerosis (ALS), Amyotrophic lateral sclerosis/Parkinsonism-dementia complex, anti-IgLON5-related tauopathy, Caribbean Parkinsonism, Chronic traumatic encephalopathy, Diffuse neurofibrillary tangles with calcification, Down syndrome, Familial British dementia, Familial Danish dementia, Niemann-Pick disease, type C, Non-Guamanian motor neuron disease with neurofibrillary tangles, Postencephalitic Parkinsonism, Primary age-related tauopathy, Progressive ataxia and palatal tremor, Tangle-only dementia, Familial frontotemporal dementia and Parkinsonism, Pick's disease, Argyrophilic grain disease, Corticobasal degeneration, Guadeloupean Parkinsonism, Globular glial tauopathy, Huntington's disease, Progressive supranuclear palsy, SLC9a-related Parkinsonism, Tau astrogliopathy, etc. In some embodiments, the neurodegenerative disease is multiple systems atrophy (MSA). In some embodiments, the neurodegenerative disease is Parkinson's disease. In some embodiments, the neurodegenerative disease is Alzheimer's disease.

The templated misfolding proteins of the present disclosure may be any templated misfolding proteins that is associated with a neurological disease. Templated misfolding proteins that are associated with a neurological disease include, without limitation, prions associated with transmissible spongiform encephalopathies such as Creutzfeldt-Jakob disease (CJD), a-synuclein associated with multiple system atrophy (MSA), amyloid b associated with Alzheimer's and Parkinson's disease, tau associated with Alzheimer's and Parkinson's disease, etc.

The administering may be any form of administering that results in the templated misfolded protein being bound by molecules. In some embodiments, the administering is performed by administering the molecules to an individual having or predicted to have the neurological disease. In some embodiments, the administration is through oral administration. In some embodiments the administration is through parenteral depot. In some embodiments, the administration is intravenous. In some embodiments, the contacting involves administration of an effective dose of the molecule to an individual having or is suspected to have a neurological disease. In some embodiments, the administering involves administration of an effective dose of the molecule to an individual having or is suspected to have a neurological disease.

An effective dose as used herein means a dose sufficient to alleviate symptoms associated with the neurodegenerative disease. The term effective dose also refers to the amount of the molecules that is sufficient to effect beneficial or desired results. The effective dose will vary depending upon the subject and neurological disease being treated, the weight and age of the subject, the severity of the disease condition, the manner of administration and the like, which can readily be determined by one of ordinary skill in the art. The term also applies to a dose that will provide an image for detection by any one of the detection methods described herein. The specific dose will vary depending on the particular compound or molecule chosen, the dosing regimen to be followed, whether it is administered in combination with other compounds, timing of administration, the tissue to be imaged, and the physical delivery system in which it is carried. The effective dose is any dose that halts, reverses, or alleviates the neurological condition.

The halting, reversal or alleviation of the neurological condition may be determined by evaluating an individual having or suspecting to have a neurodegenerative disease before and after the administration for cognitive defects associated with the neurodegenerative disease. The halting, reversal or alleviation of the neurodegenerative disease may be determined by determining if the progressive, templated misfolding of proteins associated with neurodegenerative disease is impeded by, for example, using the detection methods disclosed above or herein.

The molecules may be any molecules that inhibit templated misfolding proteins, e.g., prions, from sequestrating native cellular protein and its propagating amyloid form. In some embodiments, the molecule is a molecule according to formula (I). In some embodiments, the molecule is a molecule according to formula (II). In some embodiments, the molecule is a molecule according to formula (III). In some embodiments, the molecule is a molecule according to formula (IV). In some embodiments, the molecule is a molecule according to formula (V). In some embodiments, the molecule is a molecule according to formula (VI). In some embodiments, the molecule is a molecule according to formula (VII). In some embodiments, the molecule is a molecule according to formula (VIII). In some embodiments, the molecule is a molecule according to formula (IX). In some embodiments, the molecule is a molecule according to formula (X). In some embodiments, the molecule is a molecule according to formula (XI). In some embodiments, the molecule is a molecule according to formula (XII). In some embodiments, the molecule is a molecule according to formula (XIII)(a). In some embodiments, the molecule is a molecule according to formula (XIII)(b). In some embodiments, the molecule is a molecule according to formula (XIII)(c). In some embodiments, the molecule is a molecule according to formula (XIII)(d). In some embodiments, the molecule is a molecule according to formula (XIII)(e). In some embodiments, the molecule is a molecule according to formula (XIII)(f). In some embodiments, the molecule is a molecule according to formula (XIV)(a). In some embodiments, the molecule is a molecule according to formula (XIV)(b). In some embodiments, the molecule is a molecule according to any one of compounds 1-821 in FIG. 6. In some embodiments, the molecule is any of the compounds discussed above.

In some embodiment, the molecule of the present disclosure is characterized by a planar core comprised of one or two rings which are optionally heterocyclic and an accessible molecular conformation allowing three or more of the molecules to self-associated in a repeating, parallel-displaced stack along the stacked proteins. In some embodiments, the molecule is further characterized by a configuration shown in FIG. 3 where a distance between an atom within adjacent planar core is 3.3-3.5 Å. In some embodiments, molecule is further characterized by a configuration as shown in FIG. 4, wherein a distance between equivalent atoms on two adjacent molecules is 4.8 Å. In some embodiments, the molecule is further characterized by a configuration as shown in FIG. 5, wherein an angle (θ) between a line defined by two equivalent atoms on adjacent molecules and a line perpendicular to planes of the planar core is 44°. In some embodiments, molecule is further characterized by a configuration as shown in FIG. 4, wherein a minimal distance between any atom within the plane of the core and an equivalent atom within adjacent molecule bound to a stacked protein is 3.2-3.6 Å.

In some embodiments, the molecules which bind stacked proteins is further characterized by substituents forming non-covalent interactions with the stacked proteins which interactions are selected from the group consisting of hydrogen bonds, Van der Waals contacts, pi-pi interactions, and chalcogen bonds. In some embodiments, the interactions are hydrogen bonds. In some embodiments, the interactions are Van der Waals contacts. In some embodiments, the interactions are hydrogen bonds. In some embodiments, the interactions are pi-pi interactions. In some embodiments, the interactions are hydrogen bonds. In some embodiments, the interactions are chalcogen bonds.

In some cases, the molecules of the present disclosure are uniquely suited to interact with templated misfolded proteins associated with neurodegenerative diseases because the molecules form pi-pi interactions with each other thereby stacking with each other and with stacked proteins. In some case, pi-pi interactions are achieved by having: a planar core comprised of one or two rings which are optionally heterocyclic and an accessible molecular conformation allowing three or more of the molecules to self-associated in a repeating, parallel-displaced stack along the stacked proteins, a distance between an atom within adjacent planar core is 3.3-3.5 Å, a distance between equivalent atoms on two adjacent molecules is 4.8 Å, an angle (θ) between a line defined by two equivalent atoms on adjacent molecules and a line perpendicular to planes of the planar core that is 44°, and wherein a minimal distance between any atom within the plane of the core and an equivalent atom within adjacent molecule bound to a stacked protein is 3.2-3.6 Å. The molecules bind to the same site on each stacked protein thereby forming stacked molecules that are able to interrupt the propagation of stacked proteins.

### EXAMPLES

The following examples are put forth to provide those of ordinary skill in the art with a complete disclosure and description of how to make and use the present invention and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Centigrade, and pressure is at or near atmospheric.

### Example 1

Many neurodegenerative diseases (NDs) are characterized by the formation of prions which propagate into amyloid filaments. which adopt disease-specific conformations in the brain. Small molecules have recently been developed which hold promise as diagnostics and possibly therapeutics for NDs. The binding mechanisms of many of these small molecules to amyloid filaments remains unknown. Here, cryo-electron microscopy (cryo-EM) to is used determine a 2.7 Å structure of patient-derived Alzheimer's disease tau paired-helical filaments (PHFs) incubated with the GTP-1 PET probe. Cryo-EM structure reveals a novel stacked arrangement of the GTP-1 PET ligand bound to Alzheimer's disease tau filaments.

GTP-1 is bound stoichiometrically along an exposed cleft of each protofilament in a stacked arrangement that simultaneously satisfies the translational symmetry of the amyloid and pi-pi stacking of the aromatic small molecules. After modelling analysis, further calculations established that highly favorable ligand-ligand interactions facilitate this binding mode. The structure thus offers new insight into designing compounds for diagnosis and treatment of specific NDs.

The accumulation of misfolded tau proteins in the brain is a hallmark of the large subset of neurodegenerative diseases (NDs) known as tauopathies (1, 2), the most common and widely studied of which is Alzheimer's disease (AD) (3). The spread of tau deposits, known as neurofibrillary tangles (NFTs) in AD, parallels neuronal loss and cognitive impairment (4, 5) and serves as a marker for disease progression (6). Moreover, accumulation of NFTs has been shown to occur by a process in which soluble tau become prions, which are converted into ordered, stable amyloid filaments. Prions self-propagate and transmit across neurons via synaptic junctions (7-10). Prions were first identified in PrPSc, which causes Creutzfeldt-Jakob (CJD), Gerstmann-Sträussler-Scheinker (GS S) and other incurable diseases (11, 12). Structures determined by cryo-electron microscopy (cryo-EM) of tau filaments purified from patient brains have revealed that the conformation of the microtubule-binding repeat sequence comprising the cross-f3 sheet filament core varies among different NDs (13-18). This has opened up the possibility of site-specific binding of small-molecules to different tau prion conformers; here an example of a mechanism for achieving site-specificity is presented.

Small molecules, which can discriminate among amyloid proteins (19, 20) and even strains of the same prions (21, 22), have been developed. However, the mechanism of this specificity is unknown. Despite this limitation, a number of promising tau-selective PET ligands for AD have been developed and tested in vivo (23-27). Many such molecules contain heterocyclic aromatic moieties (28), including Flortaucipir, a first generation tau PET ligand which is FDA-approved and clinically available (29). While second generation PET tracers have been developed to reduce off-target binding and optimize pharmacokinetic properties (30, 31), the mechanism of specificity remains unknown, limiting their rational design, as well as the design of better diagnostics and more efficacious therapeutics for an expanding array of NDs. Current models provided by a cryo-EM structure of the PET ligand APN-1607 at low-resolution (32) and docking studies (33-36) indicate that small-molecules bind end to end and parallel to the fibril axis. As these models show heterogeneous binding sites within the tau prion fibril core, the conformational specificity is not explained. Additional co-structures that identify site-specific ligand binding modes are needed to develop models of binding to amyloid folds and advance development of conformationally specific probes.

Using cryo-EM, the structure of GTP-1 (Genentech Tau Probe 1) was determined, a high affinity (11 nM Kd), second-generation tau PET tracer which is currently in clinical trials Tau filament samples were purified from the frontal cortex of a patient with AD as described previously (13) and show high infectivity in a cell-based assay. Samples were incubated with 20 uM GTP-1 prior to vitrification. Concentrations well above the estimated Kd of 11 nM (23) were used to achieve site-saturation of the tau fibrils under cryo-EM conditions. The micrograph images and their 2D classification reveal well-resolved filaments primarily in the PHF conformation, with crossover distances ranging from 700-800 Å. A minor population of straight filaments (SF) was also identified; however, further structural characterization was not feasible due to limited abundance. Using standard helical reconstruction methods, (see Methods) a structure of the PHF was determined with an overall resolution of 2.7 Å that exhibited well-defined β-strand separation. The PHF structure is comprised of two protofilaments related by two-fold symmetry with a 2.37 Å rise and 179.45° twist, consistent with previously reported structures of PHFs prepared from AD brains (13, 15). The protofilaments form the canonical C-shaped cross-(3 fold found in AD that is comprised of the 3R and 4R tau domains (residues 306-378) and interact laterally via the antiparallel PGGGQ motif (residues 332-336). This central region is at the highest resolution at ˜2.5 Å and the periphery is at ˜3.2 Å, indicating high-resolution across the β-sheet core that exhibits well-resolved side chain densities.

Remarkably, the structure reveals strong additional density that is indicative of the GTP-1 small molecule bound to a solvent exposed cleft (residues 351-360) adjacent to the three-strand β-helix (β5-7) Notably, this density is identical in both protofilaments, indicating equivalent binding. While other densities are present around the filament core, these are poorly resolved in comparison and are similar to densities present in previously reported tau filament structures. Importantly, difference map analysis comparing the GTP-1 co-structure to a previously determined PHF map (EMDB: 0259) (15) identifies that this density is uniquely present, with no additional density in the difference map, indicating specific binding by GTP-1. Thus, in contrast to the earlier PET ligand study identifying multiple possible binding modes, it was observed only a single, well-defined binding site for GTP-1 within the structured core of tau.

The density shows GTP-1 binds tau in a 1:1 stoichiometry, with the compound stacked in a geometric repeat that precisely matches that of protein monomers in the fibril. The ligands form a parallel-displaced stack in which each GTP-1 spans three tau monomers This arrangement is in stark contrast to the previously described models which predict binding end to end and parallel to the fibril axis (32-36). Notably, the ligand resolution is similar to the adjacent filament structure (˜2.6 Å), and the density remains present at high sigma threshold values indicating near-complete occupancy. Taken together, our observations confirm that GTP-1 binds tau in a singular conformation within a conserved binding site.

An atomic model of the tau PHF was achieved by docking and refinement of a previous 3.2 Å resolution structure of PHFs solved in the absence of exogenous ligand. The overall filament structure is nearly identical to previous structures of AD PHFs (α-carbon RMSD=0.5 Å). However, small differences are seen in the sidechains of the residues lining the binding pocket, namely Lys353, Asp358 and Ile360. It is unclear whether the binding site perturbations are the result of improved resolution relative to the published, apo form, or whether they are due to ligand-induced perturbations to the cleft. To fit GTP-1 into the well-resolved ligand density, it was found that the best approach resulted from a combination of using molecular mechanics to generate conformers and density functional theory to perform constrained optimizations of dimers to capture small molecule-small molecule interactions. By comparison to the electron density and use of a clash filter (<2.5 Å) for small molecule-small molecule and small molecule-protein interactions, it was possible to rapidly converge on a suitable starting conformation for final refinement with Phenix (37). The final modeled conformer yields excellent map-model agreement and is energetically reasonable.

GTP-1 binds into a groove in the AD fibril with precise physiochemical and geometric complementarity. The binding site is comprised of strands β6 and β7, which are separated by a kink at Gly355, that creates a concave cleft which complements the convex shape of the GTP-1 stack. It was identified that within a uniform parallel-displaced stack, each molecule of GTP-1 binds across 3 β-strands, making direct contacts with Gln351 in strand 1, Gln351, and Lys353 in strand 2 and Ile360 in strand 3, as well as the backbone between Gln351 and Lys353 in strands 1 and 2. Notably, a short portion of GTP-1 is parallel to the filament and intercalates between two β-strands. Although the site is comprised of primarily polar residues, there is precise matching between the apolar portions of their sidechains and the apolar portions of the small-molecule. The aliphatic carbon of Ile360 contacts C7 of the phenyl ring and the apolar carbons of the Gln353 sidechain line the section of the pocket occupied by the relatively non-polar fluoroethyl tail. Specific hydrogen bonding interactions also make prominent contributions to the binding of GTP-1. Lys353 lies at the bottom of the binding groove, where it forms a bifurcated hydrogen bond with the benzimidazole nitrogen (2.8 Å N—N distance) and the pyrimido nitrogen (3.4 Å) of GTP-1, satisfying the hydrogen bonding potential of the buried polar atoms within the tricyclic aromatic ring. Lys353 also completes its hydrogen bonding potential by forming a strong salt bridge with Asp358 in the same strand, and a weaker hydrogen bond with Asp358 in the adjacent strand. The oxygen of the Gln351 sidechain is well positioned to make a non-canonical hydrogen bond with the C—H bond of the beta carbon of the fluoroethyl tail, which points inward the fibril backbone. This tail orientation allows for close van der Waals contacts with backbone atoms in two strands as well as the interaction with the sidechain of Gln351.

A clear observation from the modeled GTP-1 ligand is that the stacked heterocycles are situated at an optimal distance for pi-pi stacking (3.3-3.5 Å). To assess the favorability of these pi-pi interactions, Hartree-Fock London Dispersion calculations were performed (38). The aromatic and non-aromatic regions of GTP-1 (aromatic, pyrimido[1,2-a]benzimidazole, and non-aromatic, 2-fluoro-4-ethylpiperidine) make distinct contributions to the overall interaction. The major component (57%) indeed originates from the aromatic-aromatic interaction, whereas the smallest contribution comes from the cross interaction of the non-aromatic region with the aromatic region (19%), and the remainder comes from the non-aromatic-non aromatic interaction (24%). Given that these subunits (aromatic and non-aromatic) have similar surface area (340 Å2 and 315 Å2), this speaks to the electronic favorability of stacking aromatic molecules, as opposed to non-aromatic molecules, and this is before entropy considerations, which will also favor more rigid, aromatic molecules. It was hypothesized that the tilt of the aromatic region of GTP-1 relative to the amyloid backbone is a geometric consequence of the optimal pi-pi stacking distance, as well as the 4.77 Å repeat of the amyloid (and the helical twist, although this is negligible over short assemblies). Approximating the tilt as a simple cosine relationship between these two values (44°) fits the observed data. Given the commonality of heterocyclic aromatics in small molecules that bind amyloids and the constancy of the rise in amyloids, the adoption of a tilted heterocycle relative to the amyloid backbone, which allows for significant favorable pi-pi interactions between small molecules while maintaining the translational symmetry of the amyloid, will be a common motif in such systems.

This structure is a powerful strategy for discovery and design of small molecules that bind with high affinity to amyloids in both a sequence and conformation-specific manner. Filaments present a special challenge for small-molecule design, as their accessible surfaces tend to be relatively flat. This limits the amount of surface area potentially lost upon binding of a monomeric small molecule, hence the propensity for docking studies to show face-on binding of flat small molecules to the amyloid. Although GTP-1 forms a number of favorable contacts with the amyloid, the surface area lost upon binding a single monomer is negligible. However, when 2 GTP-1 molecules stack, the overall loss of surface area increases to 85 Å2, most of which is the apolar face of GTP-1, creating a large driving force associated with the burial of hydrophobic groups. This effect is not observed when 2 monomers are separated by an unliganded binding site suggests the system may be cooperative. To further examine this cooperativity, single point DFT calculations for binding of one, two, and three molecules of GTP-1 to five strands of a truncated model (residues 351-360) of tau we taken. Although the accuracy of the calculations is intrinsically limited due to their static nature and lack of explicit solvation, trends are gleaned. Notably, the binding energy of a single tracer against the five strands is the same in all three potential binding sites, suggesting that protein-small molecule interactions are limited to the three strands crossed by GTP-1. For two tracers bound in adjacent sites, the energy is the sum of the protein-small molecule binding energies and the small molecule-small molecule dimerization energy, indicating positive cooperativity. The same trends continue with three tracers, the minimal model for an extended stack, suggesting the calculations are relevant to the overall assembly. In contrast, two tracers separated by an unliganded binding site (a minimal model for sparse binding) shows no favorable small molecule-small molecule binding energy.

The positive cooperativity observed in these models suggests that although this structure was obtained with a concentration of GTP-1 (2011M) higher than the measured IC50 (22 nM) (23), such assemblies of pi-stacked small molecules interacting with amyloids are likely relevant to the behavior of similarly disposed ligands. Moreover, this observed behavior, that both protein-small molecule and small molecule-small molecule interactions are local and that the latter are positively cooperative, is analogous to other, well-studied biological systems. These systems, including the random coil to helix transition of a polypeptide or the binding of dye molecules to DNA, are well-described by mathematical models (39-41), which suggests a route forward to better understanding the thermodynamic and kinetic behavior of small molecule-amyloid interaction under physiological conditions. Templated assembly and symmetry matching have also been observed in the assemblies of similar aromatic molecules with globular proteins, although the limited size of the binding pockets limit the assembly size to a maximum of four molecules (42-45).

Rather than binding to a non-descript surface along a uniform beta-sheet, the strong geometric and physical complementarity between GTP-1 and this AD-specific cleft likely imparts considerable specificity. The local architecture of Gln351 to Ile360 that comprise the GTP-1 binding site is markedly different in filament structures of other tauopathies; in many cases, the key residues that form close contacts in the AD structure are either not solvent-exposed or instead form a convex surface as opposed to the concave cleft suitable for binding. Although CTE protofilaments have a C-shaped architecture similar to AD, this region of the CTE filament structure is defined by a much shallower angle formed by the kink at Gly355. This causes Ile360 to shift ˜3 Å further from Gln351 than in the AD structure, which would result in the loss of the apolar interaction between Ile360 and C7 of the GTP-1 phenyl ring. This capping interaction, along with the close non-polar contacts between the fluoroethyl tail at the opposite end of GTP-1 and the AD amyloid filament may serve to prevent positional and conformational heterogeneity, thereby facilitating ligand stacking. Based on the structural differences between the known tau conformers, it was predicted that GTP-1 will bind specifically to AD filaments. While it is possible that binding to other conformers may occur, it would likely involve an alternate mode of binding, perhaps at a different sequence in the tau filament.

Symmetry matching as observed in the structure of GTP-1 bound to PHFs from a patient with AD may provide a powerful strategy to increase the druggability of available binding sites in filaments. As small changes to the binding site likely confer a large effect on the binding of GTP-1, designing small-molecule compounds with high specificity and affinity for a single site within the amyloid filament conformation is feasible. This analysis suggests that in the development of future tools for diagnostics and, potentially, therapeutics, an emphasis should be placed on heterocycles that stack favorably in the context of the amyloid translational constraint and on achieving shape and electrostatic synergy with the targeted binding cleft. Understanding not only the amyloid assembly as a supramolecular entity, but also the small molecule, opens a new route to designing amyloid filament binders.

Drugs generally form discrete molecular associations that result in a therapeutic interaction. This occurs when drugs bind to one or more biological targets based on complementary shape, character and/or the reactivity of their surfaces. In some cases, the result is a binary drug-target complex which alters the conformation, activity or fate of the target. In still others, multiple (identical or different) small molecules bind co-operatively at multiple discrete positions within a target complex with a net affinity or effect that is greater than the sum of individual binding events (e.g. in GPCRs: Lu, et al. Structural basis for the cooperative allosteric activation of the free fatty acid receptor GPR40 (2017) Nat Struct Mol Biol 24, 570-577; type III kinase inhibitors Martinez, et al. (2020). Avoiding or Co-Opting ATP Inhibition: Overview of Type III, IV, V, and VI Kinase Inhibitors. In: Shapiro, P. (eds) Next Generation Kinase Inhibitors. Springer, Cham.) In rare cases, two or three molecules of the same ligand have been observed to bind within a singular site in a target complex while engaging in productive interactions with each other (supramolecular dimer of small molecule ligands: Shokat, K. M. A drug-drug interaction crystallizes a new entry point into the UPR. Mol. Cell 38, 161-163 (2010); supramolecular trimer of ligands: Stornaiuolo, M., De Kloe, G., Rucktooa, P. et al. Assembly of a π-π stack of ligands in the binding site of an acetylcholine-binding protein. Nat Commun 4, 1875 (2013). dimers leveraged for drug discovery: Allen, et al. bioRxiv 2022.05.23.493001, https://doi.org/10.1101/2022.05.23.493001.) The reversible intermolecular interactions between monomers in these supramolecular dimeric or trimeric complexes counter the entropic penalty for binding two distinct molecules in the same site at the same time.

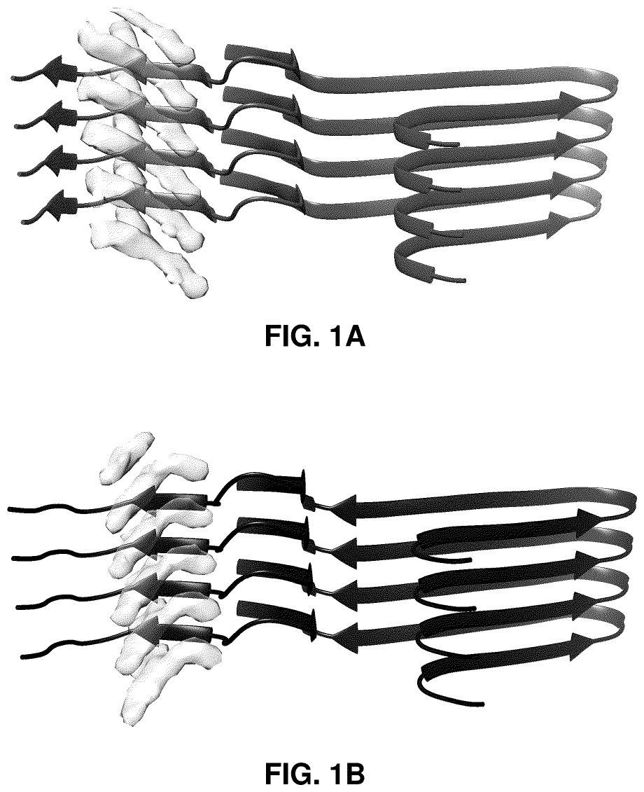

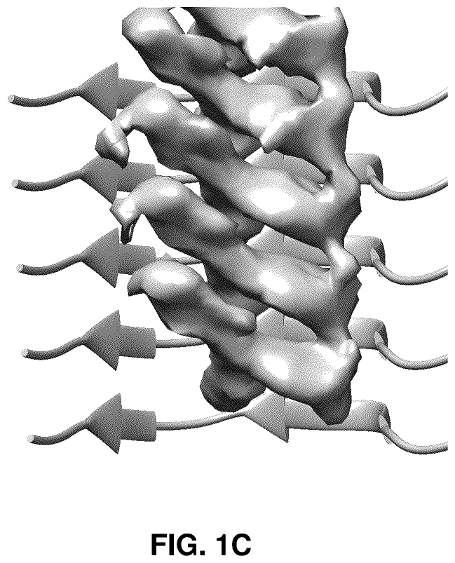

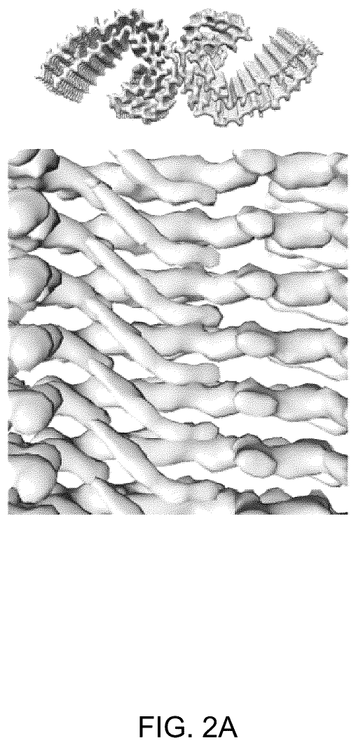

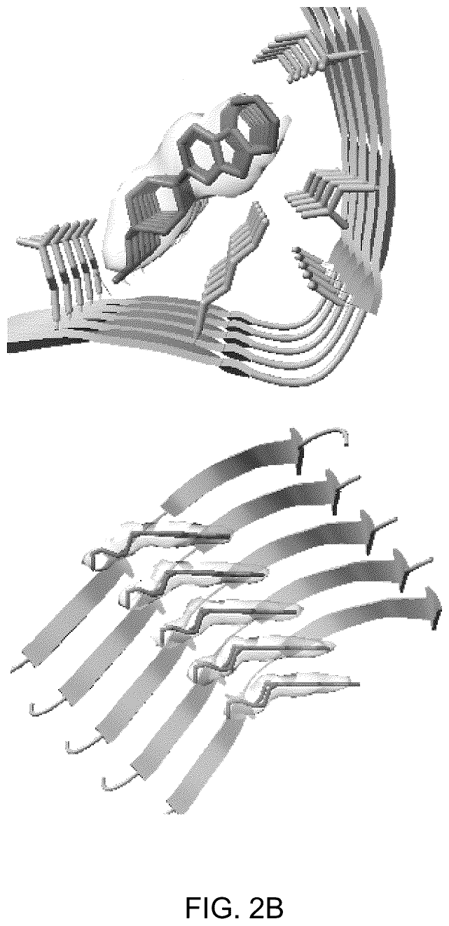

Supramolecular polymers assemble from monomers spontaneously from appropriately disposed monomers and maintain their polymeric properties in solution (de Greef, T., Meijer, E. Supramolecular polymers. Nature 453, 171-173 (2008). They are distinguished from supramolecular dimers and trimers by an increased number of monomer subunits which self-associate. Following the observation of an unprecedented supramolecular polymer assembly of an α-synuclein prion inhibitor bound to amyloid fibrils of the α-synuclein (FIG. 1), It was discovered that supramolecular polymers of small molecule monomers are uniquely suited to interact with the supramolecular assemblies of proteins which feature in degenerative diseases of the central nervous system (CNS). Owing to their helical symmetry, fibrillar oligomers and polymers typically lack a discrete druggable binding pocket. Drugs and diagnostic ligands which are large enough to span long binding channels would be favored to produce sufficient affinity to affect a pharmacologically relevant change in the activity of the fibrillized protein, but such molecules are not adequately permeable to traverse the blood-brain barrier. Supramolecular polymers composed of small monomers which can pass through the blood-brain barrier and assemble in the presence of the ordered α-synuclein aggregates that feature in multiple system atrophy (MSA) has been identified. It was demonstrated that the generality of our approach to other proteins that feature in neurodegenerative disease by establishing the binding of another supramolecular polymer: GTP-1 to paired helical filaments of tau from Alzheimer's disease (FIG. 2). As such, a general class of supramolecular polymers have been identified as modifiers and diagnostic agents for the treatment of diseases such as Alzheimer's disease, Parkinson's disease and MSA which feature the ordered accumulation of misfolded proteins.

### Example 2

### MSA Efficacy Study Protocol

Subjects: 42 (21 male and 21 female) M83 mice bred in the Institute's facility at the University of California, San Francisco were used for these experiments. M83 mice (B6/C3 background) express human alpha synuclein with the A53T mutation under the control of the mouse prion promoter. Mice were housed in an AAALAC accredited facility maintained in temperature controlled environment on a 12 hour light:dark cycle. They were given ad libitum access to food and water for the duration of the experiment. All procedures have been approved by the IACUC at the University of California, San Francisco.

Procedure: Mice were counterbalanced to experimental groups (18 control animals and 24 drug treatment animals) at 9 weeks of age and all were hand inoculated into the thalamus prior to 10 weeks of age with a cell lysate (30 μl of 1 mg/ml MSA cell lysate) from HEK cells stably propagating human MSA prions. On the day of inoculation, mice in the drug treatment condition began receiving chow formulated with the compound under investigation, while control animals received chow with no drug. Where indicated (i.e. efficacy studies of compound 3), 1-aminobenzotriazole was added to both control and test chow, delivering approximately 50 mg/kg/d of the pan-cytochrome p450 inhibitor to all mice in the study. Throughout the experiment, animals received daily health checks.

On day 14 extra animals were collected from each group so that 12 experimental animals remained in the control group and 12 experimental animals plus 6 PK animals remained in the drug treatment group. On day 49, serial bleeds and terminal collections of blood and brain were conducted on 3 males and 3 female mice from the PK group in order to assess drug levels in blood and brain. Beginning on day 50, daily neurological evaluations were added to the daily health checks. All evaluations were conducted by two individuals blind to the animals' treatment conditions.

When neurological signs developed (including proprioceptive deficits, ataxia, bradykinesia, loss of the righting reflex or loss of grasping capabilities), they were allowed to progress, consistent with the presence of a neurodegenerative disease. Then mice were sacrificed and their brains assessed for prion infectivity in a cell assay, to verify mice had been collected with neurological disease. Time (days post-inoculation) until the appearance of neurological signs was the primary experimental endpoint. The survival curves for control versus treated mice were compared using a Mantel-Cox (log-rank) statistical test. Numerical survival benefit represents median survival until the appearance of neurological signs of test group divided by median survival in the control group.

### Example 3

### α-synuclein Prion Inoculum Preparations

### Preparation of MSA-A Inoculation Material

A fragment of the basal ganglia (BG) from MSA patient PD080 was resuspended in 9 volumes of PBS (ml/g of brain) and homogenized 3×12 seconds using a probe homogenizer (Thomas Scientific) with disposable Omni Tip plastic homogenizing probes (Thomas Scientific Cat No 3409Y77) to generate a 10% brain homogenate (10% PD080-BH). Mice hemizygous for the full-length human A53T alpha synuclein transgene (B6; C3-Tg(Prnp-SNCA*A53T)83Vle/J), commonly referred to as M83 mice ((Hasson et al. Neuron 34(4)), were inoculated in the thalamus with 30 μL/mouse of 1% PD080BG-BH. The brains of the inoculated mice were collected when they exhibited neurological signs. The latter were pooled and homogenized as described above to make a 10% BH. An aliquot of the latter BH was diluted to 1% and inoculated in hemizygous M83 mice as described above for the first round of inoculations. The brains of these mice were collected when the mice exhibited neurological symptoms. The brains were pooled and homogenized to generate a 10% BH, termed SHOF6. The SHO6 10% BH was clarified of insoluble debris by centrifugation at 800×g, for 5 minutes. The resulting supernatant was collected and the protein concentration of this clarified BH (SHO6-CBH) was determined by bicinchoninic acid assay (BCA) (Thermo Fisher Cat No. 23227). HEK293 cells stably expressing a C-terminally YFP-tagged full-length wild-type α-synuclein (1-140) (S104) were transfected with SHO6-CBH using lipofectamine 2000 (LF2K) (Thermo Fisher Cat No. 11668500). Briefly, 0.1 leg of SHO6-CBH were mixed with 0.15 ul of LF2K in 2.5 μL PBS per well of a 384-well plate and incubated for 90 min at room temperature (RT). The transfection mixture was subsequently diluted by adding 7.5 μL of Opti-MEM (Thermo Scientific Cat No 31985070) and the transfection complexes added to a single well of a 384-well plate containing 3000 S104 cells. After 3 days the cells were harvested, counted and plated at a density of 1 cell/well in a 96-well plate. Clonal cells containing YEP-positive synuclein aggregates were selected and termed A8 cells. Following the expansion of these A8 cells, a cell lysate was prepared by freeze thaw. The cells from a confluent T175 flask were harvested in 1 ml of PBS containing 1× protease inhibitor cocktail by scraping. The cell suspension was subjected to 4 cycles of freezing in liquid nitrogen for 5 minutes followed by thawing at 37° C. for 5 minutes. The resulting cell lysate was centrifuged at 2000 RPM for 10 minutes to remove the cell debris and the protein concentration of the resulting MSA-A lysate was determined by BCA assay and aliquots frozen for future use.

### Preparation of MSA-B Inoculation Material

A fragment of the substantia nigra (SN) from MSA patient 1720 was resuspended in 9 volumes of PBS (ml/g of brain) and homogenized 3×12 seconds using a probe homogenizer (Thomas Scientific) with disposable Omni Tip plastic homogenizing probes (Thomas Scientific Cat No 3409Y77). The resulting 10% brain homogenate (BH) was clarified of insoluble debris by centrifugation at 800×g for 5 minutes. The protein concentration of the clarified BH (1720SN-0311) was determined by bicinchoninic acid assay (BCA) (Thermo Fisher Cat No. 23227). HEK293 cells stably and constitutively expressing a C-terminally YFP-tagged full-length α-synuclein (1-140) carrying an A53T mutation (S208.4 cells) were transfected with 1720SN-CBH using lipofectamine 2000 (LF2K) (Thermo Fisher Cat No. 11668500). Briefly, 0.57 μg/well of 1720SN-CBH were mixed with 0.86 ul/well of LF2K in 10 μL PBS and incubated for 90 min at room temperature (RT). The transfection mixture was diluted by adding 40 μL of Opti-MEM (Thermo Scientific Cat No 31985070) and added to a single well of a 96-well plate containing 1.7×10⁴ S208.4 cells. After 3 days the cells were harvested, counted and plated at a density of 1 cell/well in a 96-well plate and clonal cells containing YFP-positive synuclein aggregates were selected and termed M3 cells. Following the expansion of these M3 cells, a cell lysate was prepared by freeze thaw. The cells from a confluent T175 flask were harvested in 1 ml of PBS containing 1× protease inhibitor cocktail (Thermo Scientific Cat No A32953) by scraping. The cell suspension was subjected to 4 cycles of freezing in liquid nitrogen for 5 minutes followed by thawing at 37° C. for 5 minutes. The resulting cell lysate was centrifuged at 2000 RPM for 10 minutes to remove the cell debris and the protein concentration of the resulting M3 cell lysate was determined by BCA assay. Subsequently, HEK293 cells stably expressing the C-terminally YFP-tagged truncated α-synuclein (1-95) carrying the A53T mutation (S501 cells) were transfected with M3 lysate using lipofectamine 2000 (LF2K). Briefly, 0.57 μg/well of M3 lysate were mixed with 0.86 ul/well of LF2K in 10 tit PBS and incubated for 90 min at RT. The transfection mixture was diluted by adding 40 μL of Opti-MEM and added to a single well of a 96-well plate containing 1.7×10⁴ S501 cells. After 3 days the cells were harvested, counted and plated at a density of 1 cell/well in a 96-well plate and clonal cells containing YFP-positive synuclein aggregates were selected and termed M1 cells. Following the expansion of these M1 cells, a cell lysate was prepared by freeze thaw. The cells from a confluent 7175 flask were harvested in 1 ml of PBS containing 1× protease inhibitor cocktail by scraping. The cell suspension was subjected to 4 cycles of freezing in liquid nitrogen for 5 minutes followed by thawing at 37° C. for 5 minutes. The resulting cell lysate was centrifuged at 2000 RPM for 10 minutes to remove the cell debris and the protein concentration of the resulting MSA-B lysate was determined by BCA assay and aliquots frozen for future use.

α-synuclein Primal Cell Assays

### MSA-A Dose Response Assay in HEK293T Cells

HEK293 cells stably expressing a Tet-inducible C-terminally YFP-tagged full length α-synuclein (1-140) carrying an A53T mutation (DSS121 cells) were plated at a density of 3000 cells per well of a 384-well black walled, clear bottom plate (Greiner Cat No 5678-1091Q) in 60 μL of complete DMEM (DMIHM (Corning Cat No 10-013-CV), 10% FBS (MR Cat No 97068-085, 0.5% Penicillin/streptomycin (Gibco Cat No 15140-122) containing 0.1 μM of tetracycline and 0.1 μg/ml Hoechst Dye (Thermo Scientific Cat No 1-13570). Transfection complexes were prepared by diluting 0.24 μg of MSA-A lysate in 5 μl of PBS per well to be transfected and 0.24 μL of Lipofectamine 2000 (LF2K) (ThermoFisher Cat No 11668500) in 5 gat: of Opti-MEM per well to be transfected in separate tubes. The pre-diluted MSA-A lysate and LF2K were subsequently mixed and allowed to incubate for 90 minutes at RT. A 10 μL aliquot of the MSA-A transfection complexes were added to each well of the 384-well plate containing the DSS121 cells using the Bravo liquid handling platform. The serial dilution of test compounds was performed using the Bravo liquid handling platform (Agilent). Briefly, 25 μL of each compound of interest was added to row A of a 96-well plate (Greiner Cat No 651261) and diluted 1:1 with DMSO. Subsequently a 3-fold serial dilution in DMSO was performed for each compound from rows B-G of the 96-well plate. The compounds were further diluted by transferring 2 μL of the serially diluted DMSO stocks to a 96-well plate containing 123 μL/well of complete DMEM to generate a working stock. Following the addition of the MSA-A transfection complexes, 10 μL, of the working compound stocks was transferred from a single well of the 96-well compound plate to 4-wells each of the 384-well cell plate using the Bravo liquid handling platform. The cells were incubated for 96 h at 37° C./5% CO2 and subsequently imaged using the InCell Analyzer 6000 (Cytiva GE). Images were captured using the FITC (488 nm excitation/525+/−20 nm emission) and DAPI (405 nm excitation/455+/−50 nm emission) filter sets to image YFP-tagged α-σynuclein and Hoechst positive nuclei respectively. FITC images were processed using the InCell Developer image analysis software to determine the intensity of pixels within YFP-positive cellular aggregates as well as the number of cells that contained YFP-positive aggregates. Additionally, images captured with the DAPI filter set were processed using InCell Developer to determine the number of cells per image. Finally, the processed data were plotted as a function of the concentration of the compound and EC50 and efficacy window values calculated from these dose response curves.

### MSA-B Dose Response Assay in HEK293T Cells

HEK293 cells stably expressing a Tet-inducible C-terminally YFP-tagged full length α-synuclein (1-140) carrying an A53T mutation (DSS121 cells) were plated at a density of 3000 cells per well of a 384-well black walled, clear bottom plate (Greiner Cat No 5678-1091Q) in 60 μL of complete DMEM (DMEM (Corning Cat No 10-013-CV), 10% FBS (VWR Cat No 97068-085, 0.5% Penicillin/streptomycin (Gibco Cat No 15140-122) containing 0.1 μM of tetracycline and 0.1 μg/ml Hoechst Dye (Thermo Scientific Cat No H3570). Transfection complexes were prepared by mixing 0.1 μg/well of MSA-B lysate with 0.15 μL/well of LF2K in PBS and allowed to incubate for 90 minutes at RT. The MSA-B transfection complexes were subsequently diluted with 4 volumes of Opti-MEM and 10 μL of the MSA-B transfection complexes were added to each well of the 384-well plate containing the DSS121 cells using the Bravo liquid handling platform. The serial dilution of test compounds was performed using the Bravo liquid handling platform (Agilent). Briefly, 25 μL of each compound of interest was added to row A of a 96-well plate (Greiner Cat No 651261) and diluted 1:1 with DMSO. Subsequently a 3-fold serial dilution in DMSO was performed for each compound from rows B-G of the 96-well plate. The compounds were further diluted by transferring 2 μL of the serially diluted DMSO stocks to a 96-well plate containing 123 μL/well of complete DMEM to generate a working stock. Following the addition of the MSA-B transfection complexes, 10 μL of the working compound stocks was transferred from a single well of the 96-well compound plate to 4-wells each of the 384-well cell plate (ie: 96-well, A1 transferred to 384-well A1, B1, A2 & B2) using the Bravo liquid handling platform. The cells were incubated for 72 h at 37° C./5% CO₂ and subsequently imaged using the InCell Analyzer 6000 (Cytiva GE). Images were captured using the FITC (488 nm excitation/525±20 nm emission) and DAPI (405 nm excitation/455±50 nm emission) filter sets to image YFP-tagged α-Synuclein and Hoechst positive nuclei respectively. FITC images were processed using the InCell Developer image analysis software to determine the intensity of pixels within YFP-positive cellular aggregates as well as the number of cells that contained YFP-positive aggregates. Additionally, images captured with the DAPI filter set were processed using InCell Developer to determine the number of cells per image. Finally, the processed data were plotted as a function of the concentration of the compound and EC50 and efficacy window values calculated from these dose response curves.

### Example 4

### Trisubstituted Oxazoles

### Amino-Pyrimidone Intermediate Synthesis

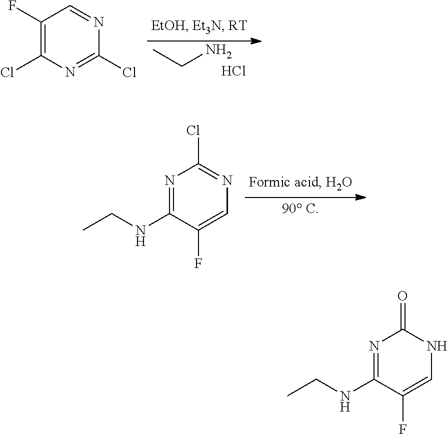

2-chloro-N-ethyl-5-fluoropyrimidin-4-amine. To a solution of 2,4-dichloro-5-fluoropyrimidine (4.00 g, 1 eq, 24.0 mmol) and triethylamine (3.15 g, 4.34 mL, 1.3 eq, 31.1 mmol) in ethanol (60 mL) was added ethylamine (2 M in THF, 14.4 mL, 1.2 eq, 28.7 mmol) under nitrogen at 0° C. over a period of 5 minutes. The reaction mixture was stirred at 0° C. for 10 minutes and at room temperature for 20 hours. The resulting mixture was concentrated under reduced pressure. The residue was diluted with an aqueous NaCl solution. The organic materials were extracted with ethyl acetate, dried over Na₂SO₄, and filtered. The filtrate was concentrated under reduced pressure, and the residue was purified by column chromatography (silica gel, 10-35% EtOAc in heptane) to afford 2-chloro-N-ethyl-5-fluoropyrimidin-4-amine (4.3 g, 24.2 mmol, 100%) as a white solid.

¹H NMR (400 MHz, CHLOROFORM-d) d ppm 1.30 (t, J=7.34 Hz, 3H), 3.57 (qd, J=7.25, 5.62 Hz, 2H), 5.18 (bs, 1H), 7.87 (d, J=2.93 Hz, 1H).

4-(ethylamino)-5-fluoropyrimidin-2(1H)-one. To a solution of 2-chloro-N-ethyl-5-fluoropyrimidin-4-amine (4.26 g, 1 eq, 24.2 mmol) in formic acid (16 g, 13 mL, 14 Eq, 0.34 mol) was added water (1.3 g, 1.3 mL, 3.0 Eq, 72 mmol), and the reaction mixture was stirred at 90° C. for 10 hours. After being cooled to room temperature, the mixture was concentrated under reduced pressure. The residue was mixed with ethanol, and the resulting precipitate was collected by vacuum filtration. The material was then washed with ethanol and ethyl acetate to afford 4-(ethylamino)-5-fluoropyrimidin-2(1H)-one (3.47 g, 22.1 mmol, 91%) as a white solid.

¹H NMR (400 MHz, DMSO-d₆) d ppm 1.15 (t, J=7.21 Hz, 3H), 3.45 (quin, J=6.85 Hz, 2H), 8.03 (d, J=6.11 Hz, 1H), 9.61 (bs, 1H), 11.70 (bs, 1H).

LCMS: rt 0.14 min. [M+H]⁺ 158.1 m/z.

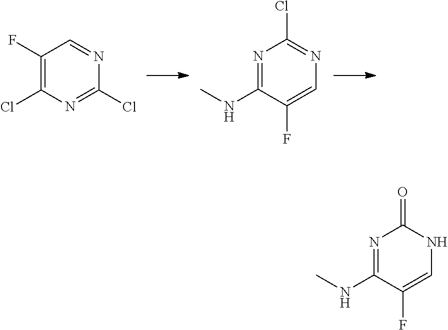

2-chloro-5-fluoro-N-methylpyrimidin-4-amine. To a stirring solution of 2,4-dichloro-5-fluoro-pyrimidine (1.0 eq) in anhydrous THF (10.0 vol) and triethylamine (1.2 eq) cooled in an ice bath was added 2 M methylamine solution in THF (1.1 eq). The mixture was stirred for 16 hours at room temperature. A saturated aqueous solution of NaHCO₃ was added and the mixture was extracted with ethyl acetate three times. The organic layers were washed with brine, dried over Na₂SO₄, and concentrated under reduced pressure to give 2-chloro-5-fluoro-N-methylpyrimidin-4-amine (80-100% yield).

¹H NMR (300 MHz, DMSO-d6): δ 8.13 (s, 1H), 8.04 (d, J=3.5 Hz, 1H), 2.84 (d, J=4.7 Hz, 3H).

5-fluoro-4-(methylamino)pyrimidin-2(1H)-one. 2-Chloro-5-fluoro-N-methylpyrimidin-4-amine (1.0 eq) was dissolved in formic acid (20.0 eq) and water (1.1 eq). The reaction mixture was stirred for 24 hours at 90° C. An additional portion of water (2.5 eq) and formic acid (9.2 eq) were then added, and the reaction was continued for another 2 days at 90° C. After cooling to ambient temperature, EtOAc was added. The resulting precipitate was collected by vacuum filtration, washed with EtOAc, and dried to afford 5-fluoro-4-(methylamino)-1,2-dihydropyrimidin-2-one (75-95% yield).

¹H NMR (300 MHz, DMSO-d₆): δ 9.92 (s, 1H), 8.06 (d, J=6.1 Hz, 1H), 2.97 (d, J=3.5 Hz, 3H).

LCMS: rt 0.12 min. [M+H]⁺ 144.2 m/z.

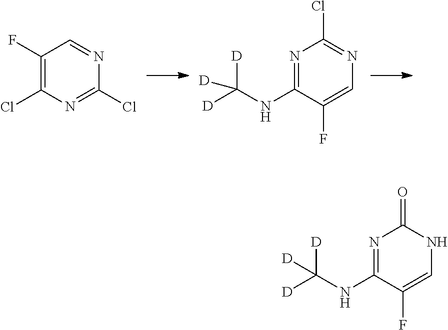 [SMILES: Fc1cnc(Cl)nc1Cl ; [2H]C([2H])([2H])Nc1nc(Cl)ncc1F ; [2H]C([2H])([2H])Nc1nc(=O)[nH]cc1F]

2-chloro-5-fluoro-N-(methyl-d₃)pyrimidin-4-amine. 2-chloro-5-fluoro-N-(methyl-d₃)pyrimidin-4-amine was prepared similarly as the preparation of 2-chloro-N-ethyl-5-fluoropyrimidin-4-amine.

¹H NMR (400 MHz, DMSO-d₆) d ppm 7.28 (bs, 1H), 7.20 (dd, J=3.42, 0.49 Hz, 1H), 1.66 (bs, 6H).

¹⁹F NMR (376 MHz, DMSO-d6) d ppm −158.11 (s, 1F). 5-fluoro-4-((methyl-d₃)amino)pyrimidin-2(1H)-one. 5-fluoro-4-((methyl-d₃)amino)pyrimidin-2(1H)-one was prepared similarly to the preparation of 4-(ethylamino)-5-fluoropyrimidin-2(1H)-one.

LCMS: rt 0.13 min. [M+H]⁺ 147.1 m/z.

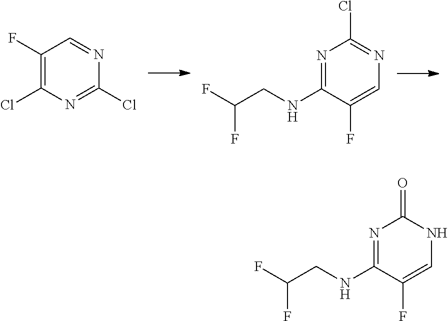 [SMILES: Fc1cnc(Cl)nc1Cl ; Fc1cnc(Cl)nc1NCC(F)F ; O=c1nc(NCC(F)F)c(F)c[nH]1]

2-chloro-N-(2,2-difluoroethyl)-5-fluoropyrimidin-4-amine. 2-Chloro-N-(2,2-difluoroethyl)-5-fluoropyrimidin-4-amine was prepared similarly as the preparation of 2-chloro-N-ethyl-5-fluoropyrimidin-4-amine.

¹H NMR (400 MHz, CHLOROFORM-d) d ppm 3.96 (tdd, J=14.61, 6.24, 4.16 Hz, 2H), 5.44 (bs, 1H), 6.00 (tt, J=55.75, 3.91 Hz, 1H), 7.99 (d, J=2.45 Hz, 1H).

4-((2,2-difluoroethyl)amino)-5-fluoropyrimidin-2(1H)-one. 4-((2,2-Difluoroethyl)amino)-5-fluoropyrimidin-2(1H)-one was prepared similarly to the preparation of 4-(ethylamino)-5-fluoropyrimidin-2(1H)-one.

¹H NMR (400 MHz, DMSO-d₆) d ppm 3.78 (t, J=14.92 Hz, 2H), 6.30 (tt, J=55.51, 3.91 Hz, 1H), 7.89 (d, J=6.11 Hz, 1H), 8.82 (bs, 1H).

LCMS: rt 0.15 min. [M+H]⁺ 194.1 m/z.

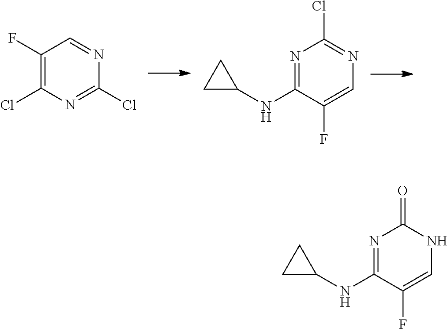 [SMILES: Fc1cnc(Cl)nc1Cl ; Fc1cnc(Cl)nc1NC1CC1 ; O=c1nc(NC2CC2)c(F)c[nH]1]

2-chloro-N-cyclopropyl-5-fluoropyrimidin-4-amine. 2-Chloro-N-cyclopropyl-5-fluoropyrimidin-4-amine was prepared similarly as the preparation of 2-chloro-N-ethyl-5-fluoropyrimidin-4-amine.

LCMS: rt 1.34 min. [M+H]⁺ 188.1 m/z. 4-(cyclopropylamino)-5-fluoropyrimidin-2(1H)-one. 4-(Cyclopropylamino)-5-fluoropyrimidin-2(1H)-one was prepared similarly to the preparation of 4-(ethylamino)-5-fluoropyrimidin-2(1H)-one.

LCMS: rt 0.12 min. [M+H]⁺ 170.1 m/z.

### Thionyl Chloride Oxazole Cyclization

### Synthesis of Compound 1

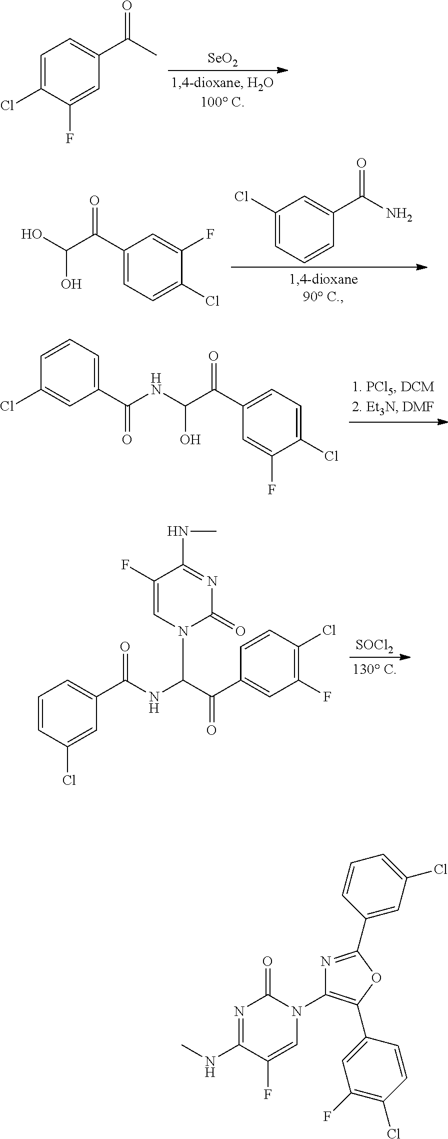

1-(4-chloro-3-fluorophenyl)-2,2-dihydroxyethan-1-one. Selenium dioxide (25.4 g, 95 Wt %, 1.5 eq, 217 mmol) was placed in water (30.0 mL) and 1,4-dioxane (300 mL) in a 500 mL medium pressure flask and heated to 70° C. until all material completely dissolved. To the reaction mixture at 70° C. was added 1-(4-chloro-3-fluorophenyl)ethan-1-one (25.0 g, 1 eq, 145 mmol). The flask was sealed and let stir at 100° C. for 21 hours. The reaction was cooled to room temperature, filtered through a pad of celite, and further eluted with EtOAc. The liquid filtrate was concentrated to a viscous brown oil, which was treated with H₂O (50 mL) and heated to 100° C. under a reflux condenser for 8 hours. The heat bath was then removed, and the mixture was stirred at room temperature for 16 hours. The resulting precipitate was collected by vacuum filtration, washed with cold water, and dried under vacuum to give 2-(4-chloro-3-fluorophenyl)-2-oxoacetaldehyde (24.1 g, 129 mmol, 89.2%) as light brown solid.

LCMS: rt 1.31 min. [M+H]⁺ 186.9 m/z.

3-chloro-N-(2-(4-chloro-3-fluorophenyl)-1-hydroxy-2-oxoethyl)benzamide. To a solution of 2-(4-chloro-3-fluorophenyl)-2-oxoacetaldehyde (2.0 g, 1 Eq, 11 mmol) in dioxane (40 mL) was added 3-chlorobenzamide (1.8 g, 1.1 Eq, 12 mmol), and the reaction mixture was heated to 90° C. for 14 hours. The reaction was then filtered hot through a pad of celite and concentrated to a brown viscous oil. The crude material was recrystallized from EtOAc/heptane and dried under vacuum to give 3-chloro-N-(2-(4-chloro-3-fluorophenyl)-1-hydroxy-2-oxoethyl)benzamide (0.738 g, 2.16 mmol, 20%) as beige solid.

¹H NMR (400 MHz, DMSO-d₆) d ppm 9.58 (d, J=7.58 Hz, 1H), 7.91 (t, J=4.89 Hz, 2H), 7.73-7.86 (m, 3H), 7.62 (d, J=8.07 Hz, 1H), 7.47-7.54 (m, 1H), 6.80 (d, J=6.60 Hz, 1H), 6.43 (t, J=7.21 Hz, 1H). LCMS: rt 2.14 min. [M+H]⁺ 344.0 m/z

3-chloro-N-(2-(4-chloro-3-fluorophenyl)-1-(5-fluoro-4-(methylamino)-2-oxopyrimidin-1(2H)-yl)-2-oxoethyl)benzamide. To a slurry of 3-chloro-N-(2-(4-chloro-3-fluorophenyl)-1-hydroxy-2-oxoethyl)benzamide (0.738 g, 1 Eq, 2.16 mmol) in DCM (20 mL) was added PCl₅ (496 mg, 95 Wt %, 1.05 Eq, 2.26 mmol). The resulting cloudy reaction mixture was stirred at 50° C. for 2 hours. The reaction was concentrated to a yellow solid and dried under vacuum to give the intermediate chloro-adduct as light-yellow solid. A solution of 5-fluoro-4-(methylamino)pyrimidin-2(1H)-one (401 mg, 1.3 Eq, 2.80 mmol) and triethylamine (655 mg, 902 μL, 3 Eq, 6.47 mmol) in DMF (20 mL) was stirred for 15 minutes at room temperature. The intermediate chloro-adduct was added, and the reaction mixture was stirred at room temperature 16 hours. The reaction was concentrated under vacuum and treated with water (10 mL). The resulting precipitate was collected by vacuum filtration, washed with cold water, and dried under vacuum to give 3-chloro-N-(2-(4-chloro-3-fluorophenyl)-1-(5-fluoro-4-(methylamino)-2-oxopyrimidin-1(2H)-yl)-2-oxoethyl)benzamide (276 mg, 591 μmol, 27.4%) as a light yellow solid.

LCMS: rt 2.21 min. [M-FH]+467.0 m/z

### 1-(5-(4-chloro-3-fluorophenyl)-2-(3-chlorophenyl)oxazol-4-yl)-5-fluoro-4-(methylamino)pyrimidin-2(1H)-one. Compound 125

3-Chloro-N-(2-(4-chloro-3-fluorophenyl)-1-(5-fluoro-4-(methylamino)-2-oxopyrimidin-1(2H)-yl)-2-oxoethyl)benzamide (58.6 mg, 1 Eq, 125 μmol) was treated with thionyl chloride (1.63 g, 1.00 mL, 109 Eq, 13.7 mmol), and the reaction mixture was stirred at 80° C. for 3 hours. The reaction was cooled to room temperature and a precipitate formed. The solid was collected by vacuum filtration, washed with EtOAc, and dried under vacuum to give 1-(5-(4-chloro-3-fluorophenyl)-2-(3-chlorophenyl)oxazol-4-yl)-5-fluoro-4-(methylamino)pyrimidin-2(1H)-one (26.8 mg, 59.7 μmol, 47.6%) as white solid.

¹H NMR (400 MHz, DMSO-d₆) δ ppm 8.68 (d, J=4.65 Hz, 1H), 8.25 (d, J=0.98 Hz, 1H), 8.16 (d, J=6.36 Hz, 1H), 8.10 (d, J=6.60 Hz, 1H), 7.86 (d, J=10.51 Hz, 1H), 7.74 (t, J=7.95 Hz, 1H), 7.66-7.71 (m, 1H), 7.60-7.66 (m, 1H), 7.43 (d, J=8.31 Hz, 1H), 2.92 (d, J=4.40 Hz, 3H).

LCMS: rt 2.43 min. [M+H]⁺ 449.0 m/z.

### Synthesis of Compound 83

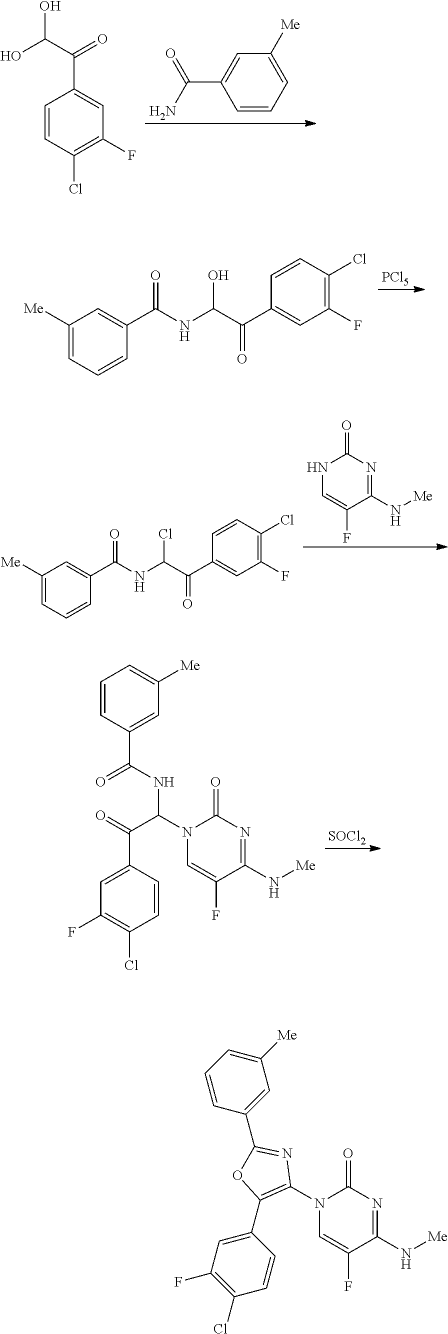

N-(2-(4-chloro-3-fluorophenyl)-1-hydroxy-2-oxoethyl)-3-methylbenzamide. 3-Toluamide (2.0 g, 14.8 mmol, 1.0 eq) and 1-(4-chloro-3-fluorophenyl)-2,2-dihydroxyethan-1-one (4.72 g, 22.2 mmol, 1.5 eq) were dissolved in dioxane (40.0 mL), and the resulting mixture was stirred at 100° C. for 2 hours. The solvent was evaporated, and the resulting material was purified by silica gel column chromatography (0-10% MeOH in DCM) to give N-(2-(4-chloro-3-fluorophenyl)-1-hydroxy-2-oxoethyl)-3-methylbenzamide (3.92 g, 12.2 mmol, 82%).

¹H NMR (300 MHz, DMSO-d6) δ 9.37 (d, J=7.9 Hz, 1H), 7.91 (dd, J=10.1, 1.6 Hz, 1H), 7.85-7.73 (m, 2H), 7.71-7.61 (m, 2H), 7.35 (dd, J=4.7, 2.2 Hz, 2H), 6.64 (s, 1H), 6.39 (s, 1H), 2.34 (s, 3H).

N-(1-chloro-2-(4-chloro-3-fluorophenyl)-2-oxoethyl)-3-methylbenzamide. N-(2-(4-Chloro-3-fluorophenyl)-1-hydroxy-2-oxoethyl)-3-methylbenzamide (2.0 g, 6.22 mmol, 1.0 eq) was dissolved in DCM (40.0 mL) and phosphorus pentachloride (1.36 g, 6.53 mmol, 1.05 eq) was added. The resulting mixture was stirred for 16 hours at ambient temperature. The solvent was removed under reduced pressure. The resulting material was suspended in hexanes and collected by vacuum filtration to afford N-[1-chloro-2-(4-chloro-3-fluorophenyl)-2-oxoethyl]-3-methylbenzamide (0.605 g, 1.78 mmol, 29%).

¹H NMR (300 MHz, DMSO-d6): δ 9.38 (d, J=7.9 Hz, 1H), 7.91 (dt, J=10.2, 3.0 Hz, 1H), 7.83-7.76 (m, 2H), 7.71-7.59 (m, 2H), 7.46-7.32 (m, 2H), 6.39 (d, J=7.9 Hz, 1H), 2.34 (d, J=4.3 Hz, 3H).

N-(2-(4-chloro-3-fluorophenyl)-1-(5-fluoro-4-(methylamino)-2-oxopyrimidin-1(2H)-yl)-2-oxoethyl)-3-methylbenzamide. N-(1-Chloro-2-(4-chloro-3-fluorophenyl)-2-oxoethyl)-3-methylbenzamide (0.605 g, 1.78 mmol, 1.0 eq) was added to a mixture of 5-fluoro-4-(methylamino)pyrimidin-2(1H)-one (0.229 g, 1.6 mmol, 0.9 eq) and sodium bicarbonate (0.747 g, 8.89 mmol, 5.0 eq) in DMF (4.6 mL) at 0° C. The reaction mixture was then stirred for 30 minutes at temperature and allowed to warm to ambient temperature with stirring for 48 hours. The solvent was evaporated, and the resulting solid was taken up in water and collected by vacuum filtration. The solid was then dissolved in CH₂Cl₂ and washed with brine three times. The organic layer was collected and dried over sodium sulfate. The solvent was removed to afford N-(2-(4-chloro-3-fluorophenyl)-1-(5-fluoro-4-(methylamino)-2-oxo-1,2-dihydropyrimidin-1-yl)-2-oxoethyl)-3-methylbenzamide (0.439 g, 0.982 mmol, 35%).

LCMS (ESI+): m/z 446.90, [M+H]⁺.

### 1-(5-(4-chloro-3-fluorophenyl)-2-(m-tolyl)oxazol-4-yl)-5-fluoro-4-(methylamino)pyrimidin-2(1H)-one. Compound 83

N-(2-(4-Chloro-3-fluorophenyl)-1-(5-fluoro-4-(methylamino)-2-oxo-1,2-dihydropyrimidin-1-yl)-2-oxoethyl)-3-methylbenzamide (0.438 g, 0.618 mmol) was dissolved in thionyl chloride (1.58 mL, 21.6 mmol, 35 eq) and stirred for 2 hours at 60° C. The solvent was removed under vacuum and the product precipitated with methanol and ethyl ether. The solid precipitate was collected by vacuum filtration and purified by silica gel column chromatography (0-10% MeOH in DCM) to afford 1-(5-(4-chloro-3-fluorophenyl)-2-(3-methylphenyl)-1,3-oxazol-4-yl)-5-fluoro-4-(methylamino)-1,2-dihydropyrimidin-2-one (0.233 g, 0.543 mmol, 87%).

LCMS (ESI+): m/z 428.86, [M+H]⁺.

¹H NMR (300 MHz, DMSO-d₆) δ 8.47 (d, J=5.0 Hz, 1H), 8.13 (d, J=6.7 Hz, 1H), 8.03-7.92 (m, 2H), 7.81-7.70 (m, 2H), 7.54-7.36 (m, 3H), 2.91 (d, J=4.6 Hz, 3H), 2.43 (s, 3H).

### Synthesis of Compound 126

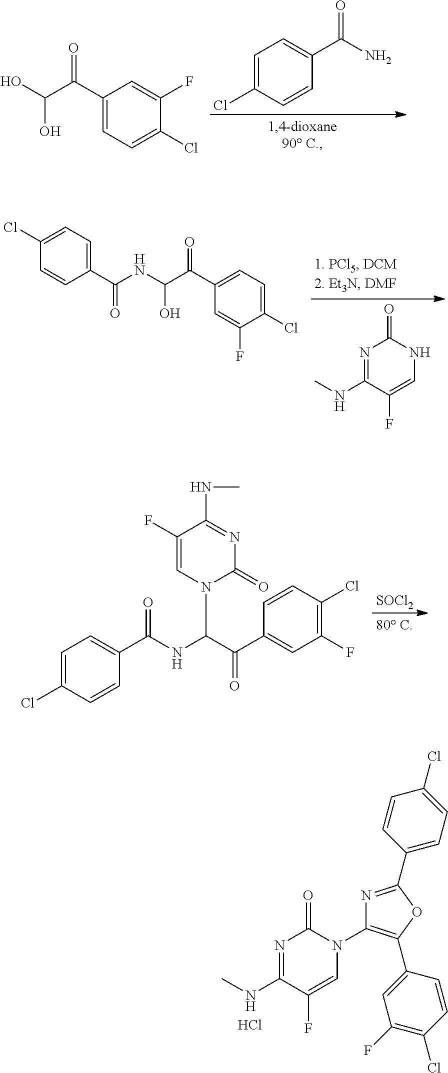

### 4-chloro-N-(2-(4-chloro-3-fluorophenyl)-1-hydroxy-2-oxoethyl)benzamide

4-Chloro-N-(2-(4-chloro-3-fluorophenyl)-1-hydroxy-2-oxoethyl)benzamide was prepared similarly to the preparation of 3-chloro-N-(2-(4-chloro-3-fluorophenyl)-1-hydroxy-2-oxoethyl)benzamide.

¹H NMR (400 MHz, DMSO-d₆) d ppm 9.52 (d, J=7.83 Hz, 1H), 7.85-7.98 (m, 3H), 7.72-7.83 (m, 2H), 7.54 (d, J=8.31 Hz, 2H), 6.76 (d, J=6.85 Hz, 1H), 6.41 (t, J=7.34 Hz, 1H).

¹⁹F NMR (376 MHz, DMSO-d6) d ppm −115.16 (s, 1F).

LCMS: rt 2.23 min. [M+Na]⁺ 363.9 m/z.

### 4-chloro-N-(2-(4-chloro-3-fluorophenyl)-1-(5-fluoro-4-(methylamino)-2-oxopyrimidin-1(2H)-yl)-2-oxoethyl)benzamide

4-Chloro-N-(2-(4-chloro-3-fluorophenyl)-1-(5-fluoro-4-(methylamino)-2-oxopyrimidin-1(2H)-yl)-2-oxoethyl)benzamide was prepared similarly to the preparation of 3-chloro-N-(2-(4-chloro-3-fluorophenyl)-1-(5-fluoro-4-(methylamino)-2-oxopyrimidin-1(2H)-yl)-2-oxoethyl)benzamide.

LCMS: rt 2.29 min. [M+H]⁺ 467.0 m/z.

### 1-(5-(4-chloro-3-fluorophenyl)-2-(4-chlorophenyl)oxazol-4-yl)-5-fluoro-4-(methylamino)pyrimidin-2(1H)-one hydrochloride. Compound 126

1-(5-(4-Chloro-3-fluorophenyl)-2-(4-chlorophenyl)oxazol-4-yl)-5-fluoro-4-(methylamino)pyrimidin-2(1H)-one hydrochloride was prepared similarly to the preparation of (5-(4-chloro-3-fluorophenyl)-2-(3-chlorophenyl)oxazol-4-yl)-5-fluoro-4-(methylamino)pyrimidin-2(1H)-one.

¹H NMR (400 MHz, DMSO-d₆) d ppm 8.54 (d, J=5.40 Hz, 1H), 8.18 (d, J=8.56 Hz, 2H), 8.10-8.14 (m, 1H), 7.72-7.79 (m, 2H), 7.67 (d, J=8.80 Hz, 2H), 7.35-7.44 (m, 1H), 2.91 (d, J=4.65 Hz, 3H).

LCMS: rt 2.55 min. [M+H]⁺ 448.9 m/z.

### Synthesis of Compound 127

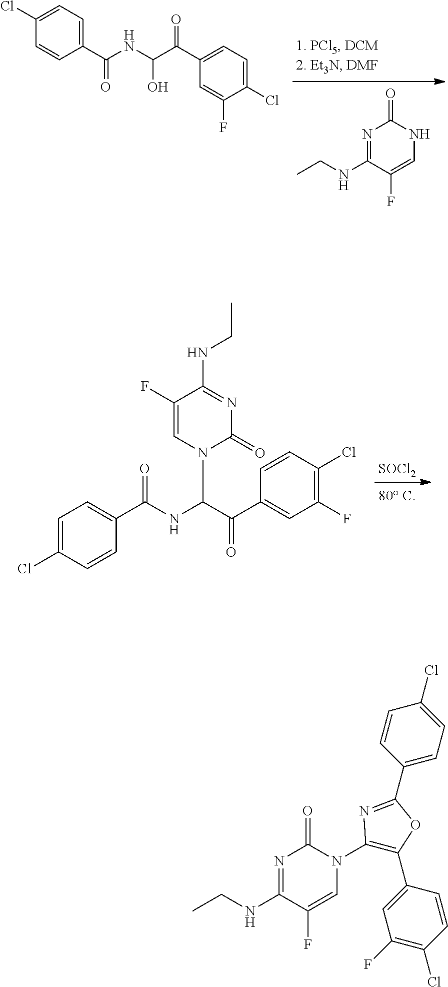

### 4-chloro-N-(2-(4-chloro-3-fluorophenyl)-1-(4-(ethylamino)-5-fluoro-2-oxopyrimidin-1(2H)-yl)-2-oxoethyl)benzamide

4-Chloro-N-(2-(4-chloro-3-fluorophenyl)-1-(4-(ethylamino)-5-fluoro-2-oxopyrimidin-1(2H)-yl)-2-oxoethyl)benzamide was prepared similarly to the preparation of 3-chloro-N-(2-(4-chloro-3-fluorophenyl)-1-(5-fluoro-4-(methylamino)-2-oxopyrimidin-1(2H)-yl)-2-oxoethyl)benzamide.

LCMS: rt 2.35 min. [M+H]⁺ 481.0 m/z.

### 1-(5-(4-chloro-3-fluorophenyl)-2-(4-chlorophenyl)oxazol-4-yl)-4-(ethylamino)-5-fluoropyrimidin-2(1H)-one. Compound 127

1-(5-(4-Chloro-3-fluorophenyl)-2-(4-chlorophenyl)oxazol-4-yl)-4-(ethylamino)-5-fluoropyrimidin-2(1H)-one was prepare similarly to the preparation of (5-(4-chloro-3-fluorophenyl)-2-(3-chlorophenyl)oxazol-4-yl)-5-fluoro-4-(methylamino)pyrimidin-2(1H)-one.

LCMS: rt 2.63 min. [M+H]⁺ 462.9 m/z.

### Synthesis of Compound 128

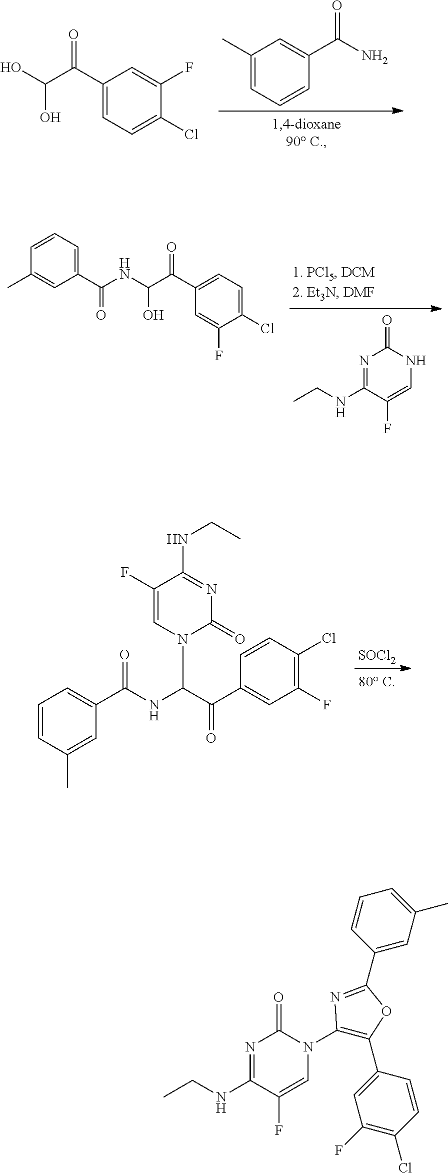

N-(2-(4-chloro-3-fluorophenyl)-1-hydroxy-2-oxoethyl)-3-methylbenzamide. N-(2-(4-Chloro-3-fluorophenyl)-1-hydroxy-2-oxoethyl)-3-methylbenzamide was prepared similarly to the preparation of 3-chloro-N-(2-(4-chloro-3-fluorophenyl)-1-hydroxy-2-oxoethyl)benzamide.

¹H NMR (400 MHz, DMSO-d₆) d ppm 9.39 (d, J=7.82 Hz, 1H), 7.90 (d, J=10.27 Hz, 1H), 7.74-7.83 (m, 2H), 7.69 (s, 1H), 7.65 (d, J=6.36 Hz, 1H), 7.28-7.41 (m, 2H), 6.66 (d, J=6.85 Hz, 1H), 6.40 (t, J=7.34 Hz, 1H), 2.33 (s, 3H).

¹³C NMR (101 MHz, DMSO-d₆) d ppm 193.56, 166.03, 155.73, 137.72, 133.25, 132.41, 131.07, 128.30, 128.05, 125.70, 125.67, 124.67, 116.57, 116.36, 74.04, 20.90.

LCMS: rt 2.14 min. [M+Na]⁺ 344.0 m/z.

N-(2-(4-chloro-3-fluorophenyl)-1-(4-(ethylamino)-5-fluoro-2-oxopyrimidin-1(2H)-yl)-2-oxoethyl)-3-methylbenzamide. N-(2-(4-Chloro-3-fluorophenyl)-1-(4-(ethylamino)-5-fluoro-2-oxopyrimidin-1(2H)-yl)-2-oxoethyl)-3-methylbenzamide was prepared similarly to the preparation of 3-chloro-N-(2-(4-chloro-3-fluorophenyl)-1-(5-fluoro-4-(methylamino)-2-oxopyrimidin-1(2H)-yl)-2-oxoethyl)benzamide.

LCMS: rt 2.32 min. [M+H]+ 461.0 m/z.

### 1-(5-(4-chloro-3-fluorophenyl)-2-(m-tolyl)oxazol-4-yl)-4-(ethylamino)-5-fluoropyrimidin-2(1H)-one hydrochloride. Compound 128

1-(5-(4-Chloro-3-fluorophenyl)-2-(m-tolyl)oxazol-4-yl)-4-(ethylamino)-5-fluoropyrimidin-2(1H)-one hydrochloride was prepared similarly to the preparation of (5-(4-chloro-3-fluorophenyl)-2-(3-chlorophenyl)oxazol-4-yl)-5-fluoro-4-(methylamino)pyrimidin-2(1H)-one.

LCMS: rt 2.60 min. [M+H]⁺ 443.0 m/z.

### Synthesis of Compound 129

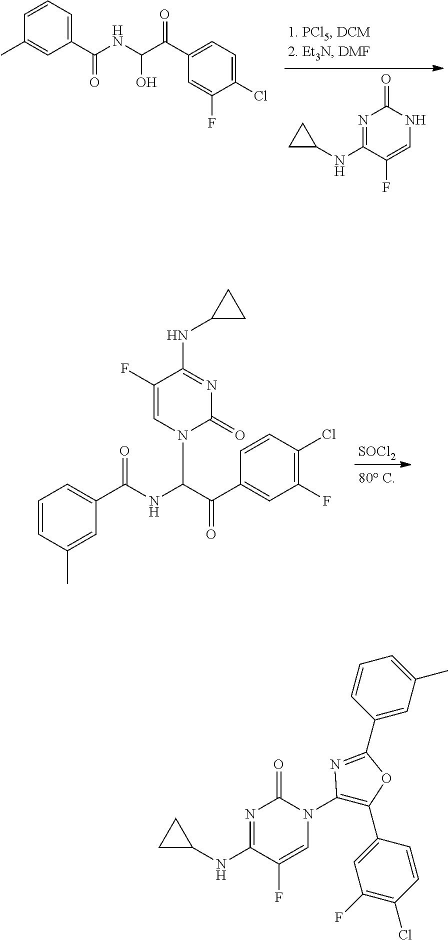

N-(2-(4-chloro-3-fluorophenyl)-1-(4-(cyclopropylamino)-5-fluoro-2-oxopyrimidin-1(2H)-yl)-2-oxoethyl)-3-methylbenzamide. N-(2-(4-Chloro-3-fluorophenyl)-1-(4-(cyclopropylamino)-5-fluoro-2-oxopyrimidin-1(2H)-yl)-2-oxoethyl)-3-methylbenzamide was prepared similarly to the preparation of 3-chloro-N-(2-(4-chloro-3-fluorophenyl)-1-(5-fluoro-4-(methylamino)-2-oxopyrimidin-1(2H)-yl)-2-oxoethyl)benzamide.

LCMS: rt 2.32 min. [M+H]⁺ 473.0 m/z.

### 1-(5-(4-chloro-3-fluorophenyl)-2-(m-tolyl)oxazol-4-yl)-4-(cyclopropylamino)-5-fluoropyrimidin-2(1H)-one hydrochloride. Compound 129

1-(5-(4-Chloro-3-fluorophenyl)-2-(m-tolyl)oxazol-4-yl)-4-(cyclopropylamino)-5-fluoropyrimidin-2(1H)-one hydrochloride was prepared similarly to the preparation of (5-(4-chloro-3-fluorophenyl)-2-(3-chlorophenyl)oxazol-4-yl)-5-fluoro-4-(methylamino)pyrimidin-2(1H)-one.

LCMS: rt 2.59 min. [M+H]⁺ 455.0 m/z.

### Synthesis of Compound 130

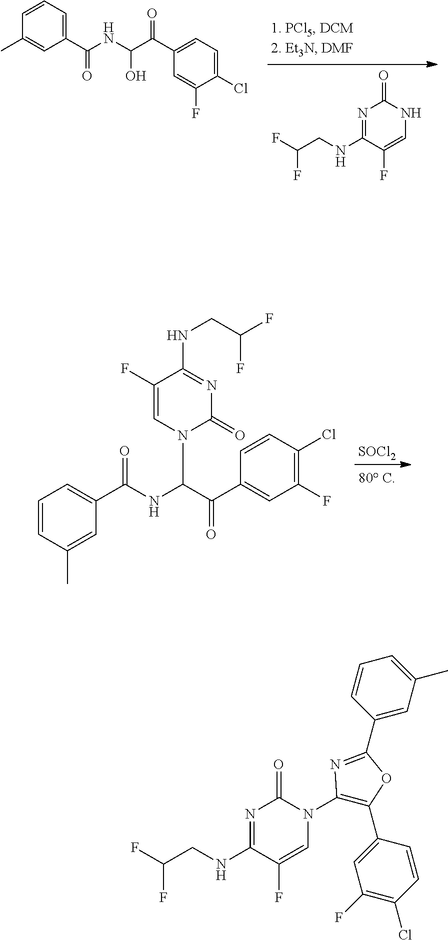

### N-(2-(4-chloro-3-fluorophenyl)-1-(4-((2,2-difluoroethyl)amino)-5-fluoro-2-oxopyrimidin-1(2H)-yl)-2-oxoethyl)-3-methylbenzamide

N-(2-(4-Chloro-3-fluorophenyl)-1-(4-((2,2-difluoroethyl)amino)-5-fluoro-2-oxopyrimidin-1(2H)-yl)-2-oxoethyl)-3-methylbenzamide was prepared similarly to the preparation of 3-chloro-N-(2-(4-chloro-3-fluorophenyl)-1-(5-fluoro-4-(methylamino)-2-oxopyrimidin-1(2H)-yl)-2-oxoethyl)benzamide.

LCMS: rt 2.35 min. [M+H]⁺ 497.1 m/z.

### 1-(5-(4-chloro-3-fluorophenyl)-2-(m-tolyl)oxazol-4-yl)-4-((2,2-difluoroethyl)amino)-5-fluoropyrimidin-2(1H)-one hydrochloride. Compound 130

1-(5-(4-Chloro-3-fluorophenyl)-2-(m-tolyl)oxazol-4-yl)-4-((2,2-difluoroethyl)amino)-5-fluoropyrimidin-2(1H)-one hydrochloride was prepared similarly to the preparation of (5-(4-chloro-3-fluorophenyl)-2-(3-chlorophenyl)oxazol-4-yl)-5-fluoro-4-(methylamino)pyrimidin-2(1H)-one.

LCMS: rt 2.62 min. [M+H]⁺ 479.0 m/z.

### Synthesis of Compound 131.

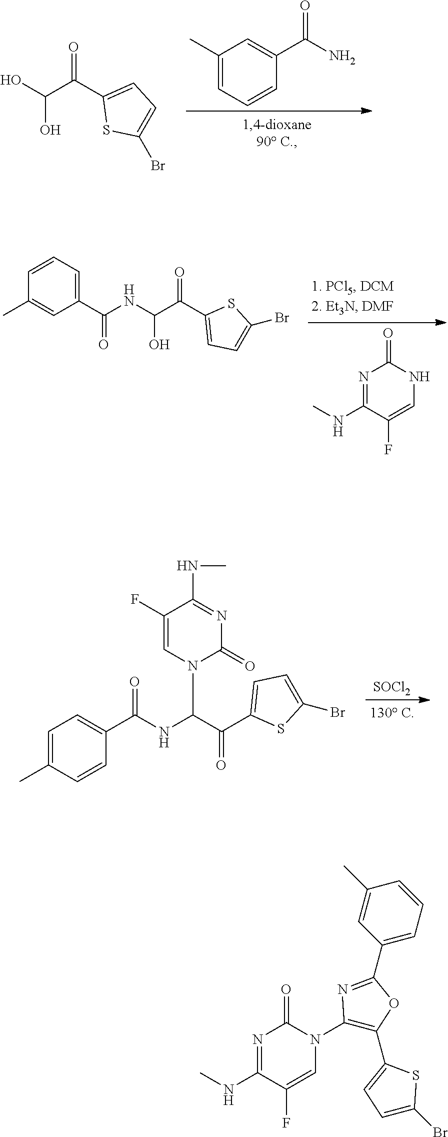

### N-(2-(5-bromothiophen-2-yl)-1-hydroxy-2-oxoethyl)-3-methylbenzamide

N-(2-(5-Bromothiophen-2-yl)-1-hydroxy-2-oxoethyl)-3-methylbenzamide was prepared similarly to the preparation of 3-chloro-N-(2-(4-chloro-3-fluorophenyl)-1-hydroxy-2-oxoethyl)benzamide.

¹H NMR (400 MHz, DMSO-d₆) d ppm 9.41 (d, J=8.07 Hz, 1H), 7.74-7.77 (m, 1H), 7.73 (s, 1H), 7.69 (d, J=6.36 Hz, 1H), 7.39 (dd, J=4.16, 0.73 Hz, 1H), 7.34-7.37 (m, 2H), 6.87 (d, J=6.60 Hz, 1H), 6.19 (t, J=7.34 Hz, 1H), 2.35 (s, 3H).

LCMS: rt 2.50 min. [M+Na]⁺ 353.0 m/z.

### N-(2-(5-bromothiophen-2-yl)-1-(5-fluoro-4-(methylamino)-2-oxopyrimidin-1(2H)-yl)-2-oxoethyl)-3-methylbenzamide

N-(2-(5-Bromothiophen-2-yl)-1-(5-fluoro-4-(methylamino)-2-oxopyrimidin-1(2H)-yl)-2-oxoethyl)-3-methylbenzamide was prepared similarly to the preparation of 3-chloro-N-(2-(4-chloro-3-fluorophenyl)-1-(5-fluoro-4-(methylamino)-2-oxopyrimidin-1(2H)-yl)-2-oxoethyl)benzamide.

¹H NMR (400 MHz, DMSO-d₆) d ppm 9.77 (d, J=8.56 Hz, 1H), 8.18 (d, J=4.65 Hz, 1H), 7.90 (d, J=6.85 Hz, 1H), 7.76 (s, 2H), 7.72 (d, J=7.34 Hz, 1H), 7.65 (d, J=4.16 Hz, 1H), 7.42 (d, J=3.42 Hz, 2H), 7.34 (d, J=8.56 Hz, 1H), 2.81 (d, J=4.65 Hz, 3H), 2.36 (s, 3H).

¹⁹F NMR (376 MHz, DMSO-d₆) d ppm −169.62-−168.03 (m, 1F).

LCMS: rt 2.11 min. [M+H]⁺ 479.0 m/z.

### 1-(5-(5-bromothiophen-2-yl)-2-(m-tolyl)oxazol-4-yl)-5-fluoro-4-(methylamino)pyrimidin-2(1H)-one. Compound 131

1-(5-(5-Bromothiophen-2-yl)-2-(m-tolyl)oxazol-4-yl)-5-fluoro-4-(methylamino)pyrimidin-2(1H)-one hydrochloride was prepared similarly to the preparation of (5-(4-chloro-3-fluorophenyl)-2-(3-chlorophenyl)oxazol-4-yl)-5-fluoro-4-(methylamino)pyrimidin-2(1H)-one.

¹H NMR (400 MHz, DMSO-d₆) d ppm 8.58 (d, J=4.40 Hz, 1H), 8.13 (d, J=6.60 Hz, 1H), 7.89 (s, 1H), 7.86 (d, J=7.82 Hz, 1H), 7.45-7.53 (m, 1H), 7.39-7.44 (m, 1H), 7.37 (d, J=3.91 Hz, 1H), 7.30 (d, J=3.91 Hz, 1H), 2.91 (d, J=4.65 Hz, 3H), 2.42 (s, 3H).

¹⁹F NMR (376 MHz, DMSO-d₆) d ppm −169.88-−166.14 (m, 1F).

LCMS: rt 2.38 min. [M+H]⁺ 460.9 m/z.

### Synthesis of Compound 132.

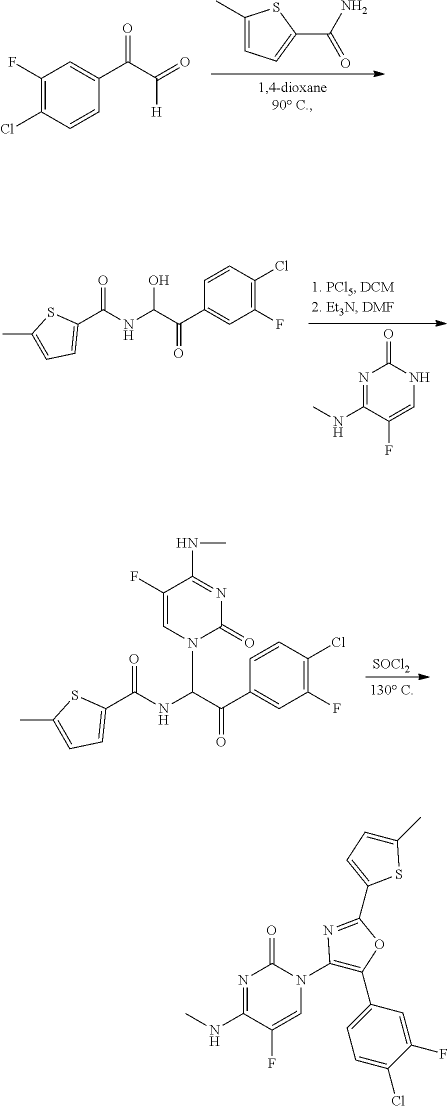

### N-(2-(4-chloro-3-fluorophenyl)-1-hydroxy-2-oxoethyl)-5-methylthiophene-2-carboxamide

N-(2-(4-Chloro-3-fluorophenyl)-1-hydroxy-2-oxoethyl)-5-methylthiophene-2-carboxamide was prepared similarly to the preparation of 3-chloro-N-(2-(4-chloro-3-fluorophenyl)-1-hydroxy-2-oxoethyl)benzamide.

¹H NMR (400 MHz, DMSO-d₆) d ppm 9.31 (d, J=8.07 Hz, 1H), 7.89 (d, J=10.27 Hz, 1H), 7.73-7.83 (m, 2H), 7.68 (d, J=3.67 Hz, 1H), 6.83 (d, J=3.67 Hz, 1H), 6.71 (d, J=6.60 Hz, 1H), 6.38 (t, J=7.34 Hz, 1H), 2.45 (s, 3H).

¹⁹F NMR (376 MHz, DMSO-d₆) d ppm −115.13 (s, 1F).

LCMS: rt 2.11 min. [M+Na]⁺ 349.9 m/z.

### N-(2-(4-chloro-3-fluorophenyl)-1-(5-fluoro-4-(methylamino)-2-oxopyrimidin-1(2H)-yl)-2-oxoethyl)-5-methylthiophene-2-carboxamide

N-(2-(4-Chloro-3-fluorophenyl)-1-(5-fluoro-4-(methylamino)-2-oxopyrimidin-1(2H)-yl)-2-oxoethyl)-5-methylthiophene-2-carboxamide was prepared similarly to the preparation of 3-chloro-N-(2-(4-chloro-3-fluorophenyl)-1-(5-fluoro-4-(methylamino)-2-oxopyrimidin-1(2H)-yl)-2-oxoethyl)benzamide.

LCMS: rt 2.10 min. [M+H]⁺ 453.0 m/z.

### 1-(5-(4-chloro-3-fluorophenyl)-2-(5-methylthiophen-2-yl)oxazol-4-yl)-5-fluoro-4-(methylamino)pyrimidin-2(1H)-one. Compound 132

1-(5-(4-Chloro-3-fluorophenyl)-2-(5-methylthiophen-2-yl)oxazol-4-yl)-5-fluoro-4-(methylamino)pyrimidin-2(1H)-one hydrochloride was prepared similarly to the preparation of (5-(4-chloro-3-fluorophenyl)-2-(3-chlorophenyl)oxazol-4-yl)-5-fluoro-4-(methylamino)pyrimidin-2(1H)-one.

¹H NMR (400 MHz, DMSO-d₆) d ppm 8.60 (d, J=4.65 Hz, 1H), 8.12 (d, J=6.60 Hz, 1H), 7.81 (d, J=3.67 Hz, 1H), 7.73 (t, J=8.19 Hz, 1H), 7.61 (dd, J=10.27, 1.71 Hz, 1H), 7.34 (dd, J=8.44, 1.83 Hz, 1H), 7.01 (d, J=3.67 Hz, 1H), 2.90 (d, J=4.65 Hz, 3H), 2.55 (s, 3H).

¹⁹F NMR (376 MHz, DMSO-d₆) d ppm −116.98-−111.63 (m, 1F), −169.61-−166.81 (m, 1F).

LCMS: rt 2.33 min. [M+H]⁺ 435.0 m/z.

### Synthesis of Compound 133

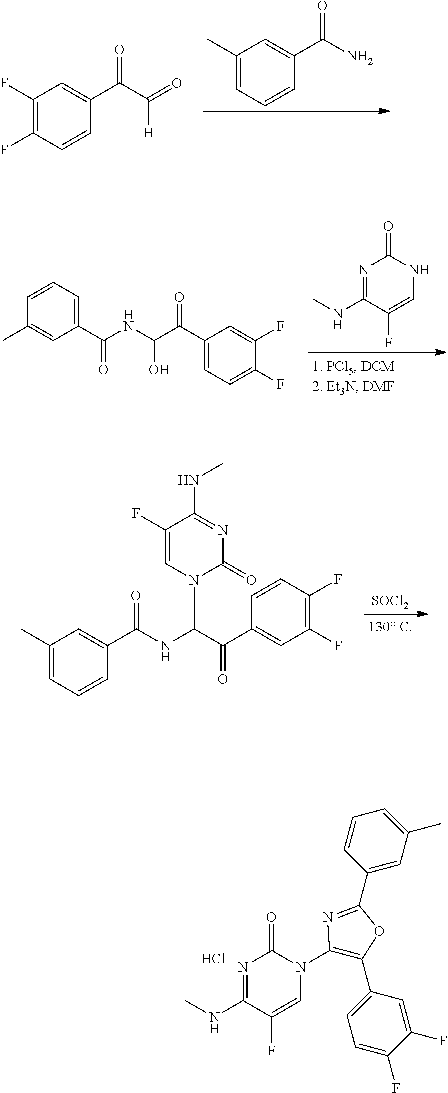

### N-(2-(3,4-difluorophenyl)-1-hydroxy-2-oxoethyl)-3-methylbenzamide

N-(2-(3,4-Difluorophenyl)-1-hydroxy-2-oxoethyl)-3-methylbenzamide was prepared similarly to the preparation of 3-chloro-N-(2-(4-chloro-3-fluorophenyl)-1-hydroxy-2-oxoethyl)benzamide.

LCMS: rt 2.02 min. [M+Na]⁺ 328.0 m/z.

### N-(2-(3,4-difluorophenyl)-1-(5-fluoro-4-(methylamino)-2-oxopyrimidin-1(2H)-yl)-2-oxoethyl)-3-methylbenzamide

N-(2-(3,4-Difluorophenyl)-1-(5-fluoro-4-(methylamino)-2-oxopyrimidin-1(2H)-yl)-2-oxoethyl)-3-methylbenzamide was prepared similarly to the preparation of 3-chloro-N-(2-(4-chloro-3-fluorophenyl)-1-(5-fluoro-4-(methylamino)-2-oxopyrimidin-1(2H)-yl)-2-oxoethyl)benzamide.

¹H NMR (400 MHz, DMSO-d₆) d ppm 8.18 (d, J=4.65 Hz, 1H), 7.88-7.98 (m, 3H), 7.76-7.84 (m, 1H), 7.73 (s, 1H), 7.60-7.71 (m, 2H), 7.44 (d, J=8.31 Hz, 1H), 7.33-7.42 (m, 2H), 2.80 (d, J=4.40 Hz, 3H), 2.35 (s, 3H).

¹⁹F NMR (376 MHz, DMSO-d₆) d ppm −129.86 (d, J=22.40 Hz, 1F), −136.65 (d, J=23.00 Hz, 1F), −169.14 (s, 1F).

LCMS: rt 2.06 min. [M+H]⁺ 431.0 m/z.

### 1-(5-(3,4-difluorophenyl)-2-(m-tolyl)oxazol-4-yl)-5-fluoro-4-(methylamino)pyrimidin-2(1H)-one hydrochloride. Compound 133

1-(5-(3,4-Difluorophenyl)-2-(m-tolyl)oxazol-4-yl)-5-fluoro-4-(methylamino)pyrimidin-2(1H)-one hydrochloride was prepared similarly to the preparation of (5-(4-chloro-3-fluorophenyl)-2-(3-chlorophenyl)oxazol-4-yl)-5-fluoro-4-(methylamino)pyrimidin-2(1H)-one.

¹H NMR (400 MHz, DMSO-d₆) d ppm 8.83 (d, J=4.65 Hz, 1H), 8.20 (d, J=6.60 Hz, 1H), 7.99 (s, 1H), 7.94 (d, J=7.58 Hz, 1H), 7.76-7.86 (m, 1H), 7.56-7.65 (m, 1H), 7.45-7.52 (m, 1H), 7.37-7.45 (m, 2H), 2.93 (d, J=4.40 Hz, 3H), 2.42 (s, 3H).

LCMS: rt 2.31 min. [M+H]⁺ 413.0 m/z.

### Synthesis of Compound 134

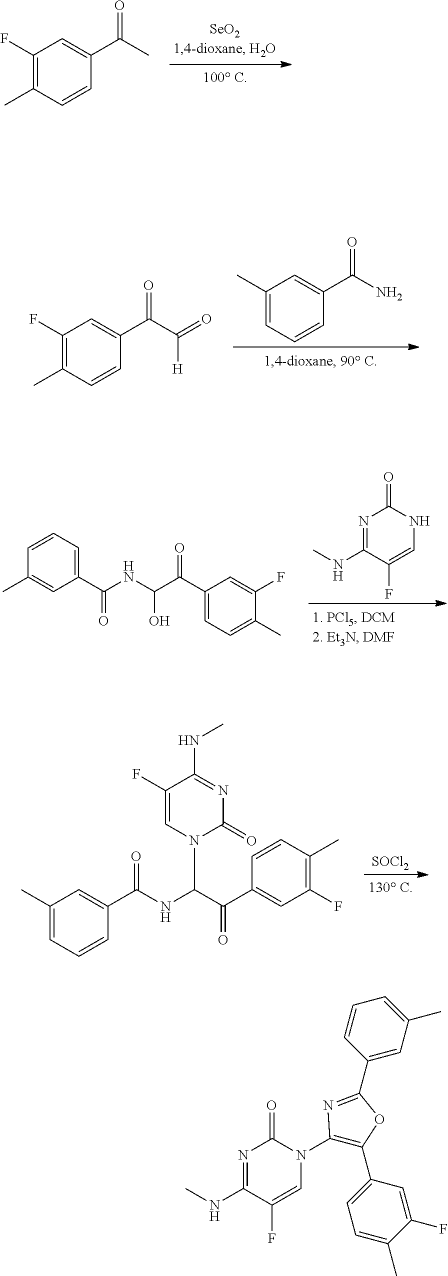

### 2-(3-fluoro-4-methylphenyl)-2-oxoacetaldehyde

2-(3-Fluoro-4-methylphenyl)-2-oxoacetaldehyde was prepared similarly to the preparation of 1-(4-chloro-3-fluorophenyl)-2,2-dihydroxyethan-1-one.

LCMS: rt 1.17 min. [M+H]⁺ 167.0 m/z.

### N-(2-(3-fluoro-4-methylphenyl)-1-hydroxy-2-oxoethyl)-3-methylbenzamide

N-(2-(3-Fluoro-4-methylphenyl)-1-hydroxy-2-oxoethyl)-3-methylbenzamide was prepared similarly to the preparation of 3-chloro-N-(2-(4-chloro-3-fluorophenyl)-1-hydroxy-2-oxoethyl)benzamide.

LCMS: rt 2.27 min. [M+Na]⁺ 324.0 m/z.

### N-(1-(5-fluoro-4-(methylamino)-2-oxopyrimidin-1(2H)-yl)-2-(3-fluoro-4-methylphenyl)-2-oxoethyl)-3-methylbenzamide

N-(1-(5-Fluoro-4-(methylamino)-2-oxopyrimidin-1(2H)-yl)-2-(3-fluoro-4-methylphenyl)-2-oxoethyl)-3-methylbenzamide was prepared similarly to the preparation of 3-chloro-N-(2-(4-chloro-3-fluorophenyl)-1-(5-fluoro-4-(methylamino)-2-oxopyrimidin-1(2H)-yl)-2-oxoethyl)benzamide.

LCMS: rt 2.23 min. [M+Na]⁺ 449.1 m/z.

### 5-fluoro-1-(5-(3-fluoro-4-methylphenyl)-2-(m-tolyl)oxazol-4-yl)-4-(methylamino)pyrimidin-2(1H)-one. Compound 134

5-Fluoro-1-(5-(4-fluoro-3-methylphenyl)-2-(m-tolyl)oxazol-4-yl)-4-(methylamino)pyrimidin-2(1H)-one was prepared similarly to the preparation of (5-(4-chloro-3-fluorophenyl)-2-(3-chlorophenyl)oxazol-4-yl)-5-fluoro-4-(methylamino)pyrimidin-2(1H)-one.

¹H NMR: (400 MHz, DMSO-d₆) d ppm 8.48 (d, J=4.4 Hz, 1H), 8.15 (d, J=6.4 Hz, 1H), 7.98 (s, 1H), 7.93 (d, J=7.6 Hz, 1H), 7.50-7.41 (m, 4H), 7.31 (d, J=8.0 Hz, 1H), 2.90 (d, J=4.8 Hz, 3H), 2.43 (s, 3H), 2.28 (s, 3H).

LCMS: rt 2.56 min. [M+Na]⁺ 409.3 m/z.

### Synthesis of Compound 135.

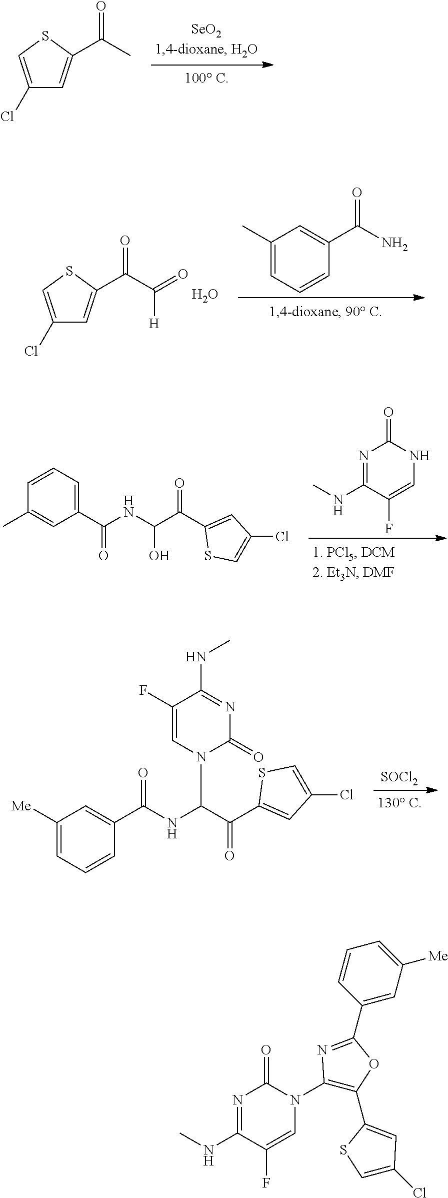

### 2-(4-chlorothiophen-2-yl)-2-oxoacetaldehyde hydrate

2-(4-Chlorothiophen-2-yl)-2-oxoacetaldehyde hydrate was prepared similarly to the preparation of 1-(4-chloro-3-fluorophenyl)-2,2-dihydroxyethan-1-one.

LCMS: rt 0.58 min. [M+Na]⁺ 214.1 m/z.

### N-(2-(4-chlorothiophen-2-yl)-1-hydroxy-2-oxoethyl)-3-methylbenzamide

N-(2-(4-Chlorothiophen-2-yl)-1-hydroxy-2-oxoethyl)-3-methylbenzamide was prepared similarly to the preparation of 3-chloro-N-(2-(4-chloro-3-fluorophenyl)-1-hydroxy-2-oxoethyl)benzamide.

LCMS: rt 2.18 min. [M+Na]⁺ 232.0 m/z.

### N-(2-(4-chlorothiophen-2-yl)-1-(5-fluoro-4-(methylamino)-2-oxopyrimidin-1(2H)-yl)-2-oxoethyl)-3-methylbenzamide

N-(2-(4-Chlorothiophen-2-yl)-1-(5-fluoro-4-(methylamino)-2-oxopyrimidin-1(2H)-yl)-2-oxoethyl)-3-methylbenzamide was prepared similarly to the preparation of 3-chloro-N-(2-(4-chloro-3-fluorophenyl)-1-(5-fluoro-4-(methylamino)-2-oxopyrimidin-1(2H)-yl)-2-oxoethyl)benzamide.

LCMS: rt 2.18 min. [M+Na]⁺ 457 m/z.

### 1-(5-(4-chlorothiophen-2-yl)-2-(m-tolyl)oxazol-4-yl)-5-fluoro-4-(methylamino)pyrimidin-2(1H)-one. Compound 135

1-(5-(4-Chlorothiophen-2-yl)-2-(m-tolyl)oxazol-4-yl)-5-fluoro-4-(methylamino)pyrimidin-2(1H)-one was prepared similarly to the preparation of (5-(4-chloro-3-fluorophenyl)-2-(3-chlorophenyl)oxazol-4-yl)-5-fluoro-4-(methylamino)pyrimidin-2(1H)-one.

¹H NMR (400 MHz, DMSO-d₆) d ppm 8.54 (d, J=4.40 Hz, 1H), 8.14 (d, J=6.60 Hz, 1H), 7.93 (s, 1H), 7.88 (d, J=7.58 Hz, 1H), 7.76 (d, J=1.47 Hz, 1H), 7.50 (d, J=1.47 Hz, 1H), 7.47 (d, J=7.58 Hz, 1H), 7.38-7.44 (m, 1H), 2.90 (d, J=4.65 Hz, 3H), 2.42 (s, 3H).

LCMS: rt 2.53 min. [M+H]⁺ 417.0 m/z.

Additional Compounds Prepared in an Analogous Manner to Compound 135:

| Compound | Exact Mass | LCMS Found | Assignment |
|---|---|---|---|
| 78 | 424.1147 | 425.1 | [M + H]+ |
| 97 | 396.0789 | 397.13 | [M + H]+ |
| 98 | 398.0991 | 399.2 | [M + H]+ |
| 99 | 396.0789 | 396.99 | [M + H]+ |
| 102 | 410.0946 | 411 | [M + H]+ |
| 111 | 430.04 | 430.97 | [M + H]+ |
| 115 | 414.0695 | 415.1 | [M + H]+ |
| 116 | 394.1241 | 395.2 | [M + H]+ |
| 117 | 396.0789 | 397.2 | [M + H]+ |
| 121 | 440.0852 | 441.12 | [M + H]+ |
| 136 | 394.1241 | 395.2 | [M + H]+ |
| 137 | 396.1056 | 397.16 | [M + H]+ |
| 138 | 410.0946 | 411 | [M + H]+ |
| 139 | 410.0946 | 411.16 | [M + H]+ |
| 140 | 414.0695 | 415.13 | [M + H]+ |
| 141 | 416.051 | 417.17 | [M + H]+ |
| 142 | 418.0644 | 419.06 | [M + H]+ |
| 143 | 424.1102 | 425.313 | [M + H]+ |
| 144 | 429.0804 | 430.17 | [M + H]+ |
| 145 | 436.1369 | 436.95 | [M + H]+ |
| 146 | 438.1259 | 439.17 | [M + H]+ |
| 147 | 444.0556 | 445.09 | [M + H]+ |
| 148 | 450.0774 | 451.08 | [M + H]+ |

### Synthesis of 1-(5-(4-chloro-3-fluorophenyl)-2-(m-tolyl)oxazol-4-yl)-5-fluoro-4-((methyl-d3)amino)pyrimidin-2(1H)-one

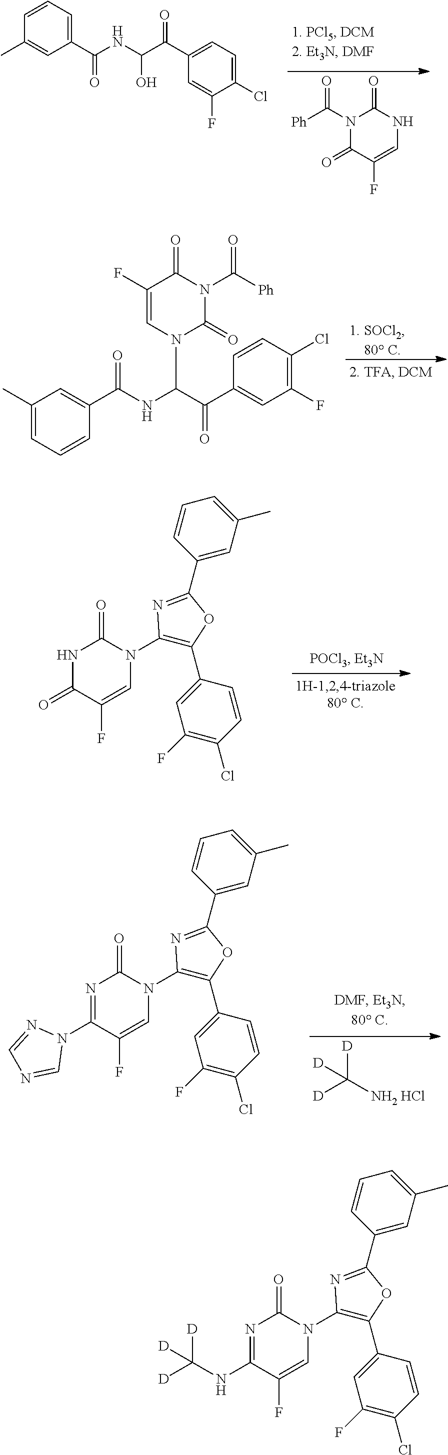

### N-(1-(3-benzoyl-5-fluoro-2,4-dioxo-3,4-dihydropyrimidin-1(2H)-yl)-2-(4-chloro-3-fluorophenyl)-2-oxoethyl)-3-methylbenzamide

To a slurry of N-(2-(4-chloro-3-fluorophenyl)-1-hydroxy-2-oxoethyl)-3-methylbenzamide (4.0 g, 12 mmol) in DCM (50 mL) was added PCl₅ (2.9 g, 95 Wt %, 1.05 Eq, 13 mmol). The resulting cloudy reaction mixture was stirred at 50° C. for 2 hours. The reaction was concentrated to a yellow solid and dried under vacuo to give the chloro-adduct as light-yellow solid which was used directly. To a 0° C. solution of 3-benzoyl-5-fluoropyrimidine-2,4(1H,3H)-dione (3.5 g, 1.2 Eq, 15 mmol) and triethylamine (3.8 g, 5.2 mL, 3 Eq, 37 mmol) in DMF (50 mL) was added the chloro-adduct, and the reaction mixture was stirred at room temperature for 16 hours. The reaction was concentrated to a viscous oil and mixed with water. The formed precipitate was collected by vacuum filtration and dried under vacuum to give N-(1-(3-benzoyl-5-fluoro-2,4-dioxo-3,4-dihydropyrimidin-1(2H)-yl)-2-(4-chloro-3-fluorophenyl)-2-oxoethyl)-3-methylbenzamide (3.7 g, 6.9 mmol, 55%) as a brown solid. This material was used crude in the next reaction.

LCMS: rt 3.61 min. [M+Na]⁺ 559.9 m/z.

### 1-(5-(4-chloro-3-fluorophenyl)-2-(m-tolyl)oxazol-4-yl)-5-fluoropyrimidine-2,4(1H,3H)-dione

N-(1-(3-Benzoyl-5-fluoro-2,4-dioxo-3,4-dihydropyrimidin-1(2H)-yl)-2-(4-chloro-3-fluorophenyl)-2-oxoethyl)-3-methylbenzamide (0.305 g, 1 Eq, 567 μmol) was treated with thionyl chloride (4.89 g, 3.00 mL, 72.5 Eq, 41.1 mmol) and stirred at 80° C. for 2.5 hours. The reaction was cooled to room temperature and concentrated to give a viscous brown oil. This material was further dried under vacuum to give crude 3-benzoyl-1-(5-(4-chloro-3-fluorophenyl)-2-(m-tolyl)oxazol-4-yl)-5-fluoropyrimidine-2,4(1H,3H)-dione as brown foam.

LCMS: rt 2.88 min. [M+H]⁺ 519.7 m/z.

Crude 3-benzoyl-1-(5-(4-chloro-3-fluorophenyl)-2-(m-tolyl)oxazol-4-yl)-5-fluoropyrimidine-2,4(1H,3H)-dione was dissolved in DCM (2 mL) and treated with TFA (1.48 g, 1.00 mL, 22.9 Eq, 13.0 mmol). The mixture was stirred at room temperature for 2 hours. The reaction was concentrated and dried under vacuum to give 1-(5-(4-chloro-3-fluorophenyl)-2-(m-tolyl)oxazol-4-yl)-5-fluoropyrimidine-2,4(1H,3H)-dione (0.161 g, 387 μmol, 68.3%) as brown foam.

LCMS: rt 2.56 min. [M+H]⁺ 416.0 m/z.

### 1-(5-(4-chloro-3-fluorophenyl)-2-(m-tolyl)oxazol-4-yl)-5-fluoro-4-(1H-1,2,4-triazol-1-yl)pyrimidin-2(1H)-one

To a 0° C. slurry of 1H-1,2,4-triazole (86 mg, 7 Eq, 1.2 mmol) in MeCN (2 mL) were added phosphoryl trichloride (68 mg, 41 μL, 2.5 Eq, 0.44 mmol) and triethylamine (0.13 g, 0.17 mL, 7 Eq, 1.2 mmol). The resulting yellow slurry was stirred at 0° C. for 30 minutes and then at 25° C. for 30 minutes. To the reaction was added a solution of 1-(5-(4-chloro-3-fluorophenyl)-2-(m-tolyl)oxazol-4-yl)-5-fluoropyrimidine-2,4(1H,3H)-dione (0.074 g, 1 Eq, 0.18 mmol) in MeCN (2 mL), and the reaction was stirred at 25° C. for 16 hours and then at 80° C. for an additional 1 hour. The mixture was cooled to room temperature, H₂O was added, and stirring was continued for 10 minutes. The mixture was then filtered and the yellow residue was washed with water (3×3 mL) and dried under vacuum to give 1-(5-(4-chloro-3-fluorophenyl)-2-(m-tolyl)oxazol-4-yl)-5-fluoro-4-(1H-1,2,4-triazol-1-yl)pyrimidin-2(1H)-one (0.073 g, 0.16 mmol, 88%) as a yellow solid.

LCMS: rt 2.50 min. [M+H]⁺ 467.0 m/z.

### 1-(5-(4-chloro-3-fluorophenyl)-2-(m-tolyl)oxazol-4-yl)-5-fluoro-4-((methyl-d3)amino)pyrimidin-2(1H)-one

To a slurry of 1-(5-(4-chloro-3-fluorophenyl)-2-(m-tolyl)oxazol-4-yl)-5-fluoro-4-(1H-1,2,4-triazol-1-yl)pyrimidin-2(1H)-one (73.0 mg, 1 Eq, 156 μmol) in DMF (3 mL) was added triethylamine (111 mg, 153 μL, 7 Eq, 1.09 mmol) followed by d3-methylamine hydrochloride (33 mg, 3 Eq, 0.468 mmol). The resulting yellow slurry was stirred at 80° C. for 1 hour. The mixture was cooled to room temperature and concentrated under vacuum. The residue was treated with H₂O and stirred for 20 minutes. The resulting precipitate was collected by vacuum filtration, washed with water, and dried under vacuum at 50° C. to give 1-(5-(4-chloro-3-fluorophenyl)-2-(m-tolyl)oxazol-4-yl)-5-fluoro-4-((methyl-d₃)amino)pyrimidin-2(1H)-one (65.7 mg, 152 μmol, 97.3%) as a yellow solid.

¹H NMR (400 MHz, DMSO-d₆) d ppm 8.43 (s, 1H), 8.13 (d, J=6.85 Hz, 1H), 8.00 (s, 1H), 7.95 (d, J=7.34 Hz, 1H), 7.68-7.80 (m, 2H), 7.45-7.53 (m, 1H), 7.35-7.44 (m, 2H), 3.33 (s, 6H), 2.43 (s, 3H).

¹⁹F NMR (376 MHz, DMSO-d₆) d ppm −116.44-−113.90 (m, 1F), −170.28-−167.34 (m, 1F).

LCMS: rt 2.41 min. [M+H]⁺ 432.0 m/z.

### Synthesis of Compound 149.

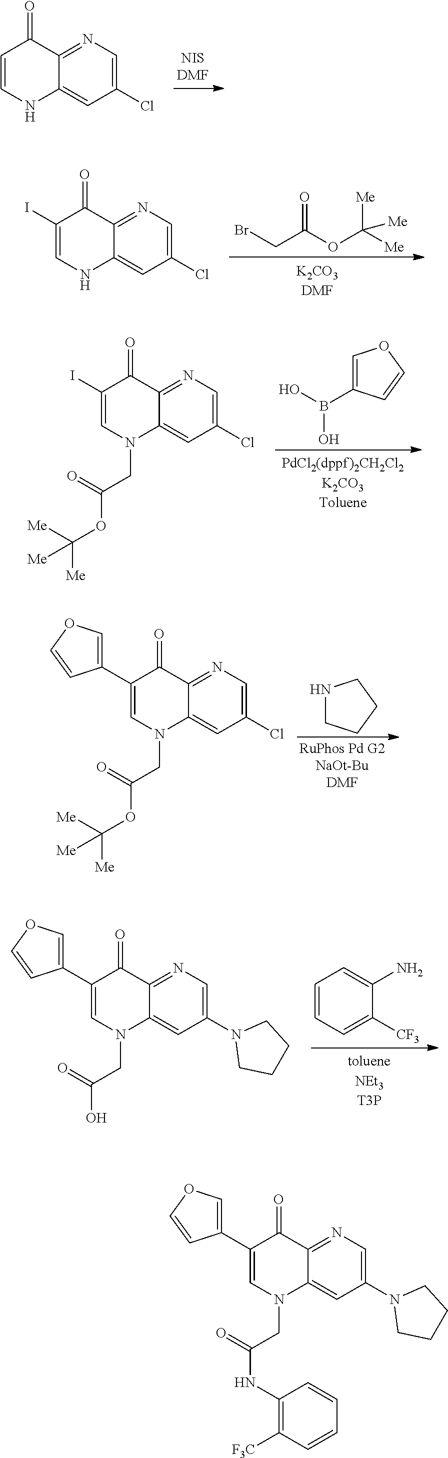

### 7-chloro-3-iodoquinolin-4-ol

7-chloroquinolin-4(1H)-one (3 g, 16.7 mmol) was dissolved in DMF (30 ml) and treated with 1-iodopyrrolidine-2,5-dione (NIS, 1.5 equiv, 5.62 g, 25 mmol) and stirred at room temperature overnight. The reaction was then diluted with water (30 ml) and the resulting precipitate was filtered off and washed with ethyl acetate and water then dried under vacuum to afford 7-chloro-3-iodoquinolin-4-ol (5.00 g, 98.0%) as a white solid.

### tert-butyl 2-(7-chloro-3-iodo-4-oxo-1,4-dihydroquinolin-1-yl)acetate

7-chloro-3-iodoquinolin-4-ol (2.3 g, 7.52 mmol) was dissolved in DMF (10 mL), then tert-butyl 2-bromoacetate (2.18 g, 11.2 mmol) and dipotassium carbonate (3.10 g, 22.5 mmol) were added and stirred at 50° C. for 16 h. The reaction was diluted with 10 ml of water and the resulting precipitate was filtered off and washed with water and a mixture of ethyl acetate and heptane (1:3) and then dried to afford tert-butyl 2-(7-chloro-3-iodo-4-oxo-1,4-dihydroquinolin-1-yl)acetate (3.10 g, 98.4%) as a pale yellow solid.

LCMS (ESI+): m/z 420.0 [M+H]+

### tert-butyl 2-[7-chloro-3-(furan-3-yl)-4-oxo-1,4-dihydroquinolin-1-yl]acetate

tert-butyl 2-(7-chloro-3-iodo-4-oxo-1,4-dihydroquinolin-1-yl)acetate (3.02 g, 7.21 mmol) was dissolved in toluene (25 mL) and then (furan-3-yl)boronic acid (1 g, 8.93 mmol), bis(cyclopentyldiphenylphosphane) dichloromethane dichloropalladium (567 mg, 686 μmol), dipotassium carbonate (2.84 g, 20.6 mmol) and water (2.5 mL) were added. The reaction was stirred at 110° C. for 2 h. Then the reaction was diluted with ethyl acetate and brine then the organic fraction was separated then dried over sodium sulfate. The solvent was removed under vacuum and purified by silica gel column chromatography eluting with (1:1) ethyl acetate and heptane to afford tert-butyl 2-[7-chloro-3-(furan-3-yl)-4-oxo-1,4-dihydroquinolin-1-yl]acetate (1.65 g, 67.0%) as a brown solid.

LCMS (ESI+): m/z 360.0 [M+H]+

### 2-[3-(furan-3-yl)-4-oxo-7-(pyrrolidin-1-yl)-1,4-dihydroquinolin-1-yl]acetic acid

tert-butyl 2-[7-chloro-3-(furan-3-yl)-4-oxo-1,4-dihydroquinolin-1-yl]acetate (1.35 g, 3.75 mmol) was dissolved in DMF (20 mL). Then pyrrolidine (1.33 g, 18.7 mmol), Chloro(2-dicyclohexylphosphino-2′,6′-diisopropoxy-1,1′-biphenyl)[2-(2′-amino-1,1′-biphenyl)]palladium(II) (292 mg, 375 μmol) and (tert-butoxy)sodium (1.10 g, 11.2 mmol) were added and the reaction heated to 110° C. for 4 h. The resulting solid was filtered off and washed with water. Then the solid was dissolved in 1N NaOH and filtered. The filtrate was then acidified with 1N HCl and the solid filtered. The solid was then washed with water, ethyl acetate and then dried under vacuum to provide 2-[3-(furan-3-yl)-4-oxo-7-(pyrrolidin-1-yl)-1,4-dihydroquinolin-1-yl]acetic acid (890 mg, 70.6%) as a yellow solid. ¹H NMR (400 MHz, DMSO-d6, ppm) d 8.51 (s, 1H), 8.33 (s, 1H), 8.09-8.06 (m, 1H), 7.68 (s, 1H), 6.90 (s, 1H), 6.76-6.74 (m, 1H), 6.13 (s, 1H), 5.08 (s, 2H), 3.40-3.30 (m, 4H), 4.60-4.40 (m, 4H).

LCMS (ESI+): m/z 339.0 [M+H]+

### 2-[3-(furan-3-yl)-4-oxo-7-(pyrrolidin-1-yl)-1,4-dihydroquinolin-1-yl]-N-[2-(trifluoromethyl)phenyl]acetamide. Compound 149

2-[3-(furan-3-yl)-4-oxo-7-(pyrrolidin-1-yl)-1,4-dihydroquinolin-1-yl]acetic acid (890 mg, 2.63 mmol) was dissolved in toluene (10 mL). Triethylamine (1.32 g, 13.1 mmol), tripropyl-1,3,5,2λ⁵,4λ⁵,6λ⁵-trioxatriphosphinane-2,4,6-trione (T3P, 5.02 g, 7.89 mmol) and 2-(trifluoromethyl)aniline (634 mg, 3.94 mmol) were then added and stirred at 40° C. for 2 h. The reaction was quenched with 1N HCl. The resulting solid was filtered then washed with water, ethyl acetate, collected by filtration and dried under vacuum to afford 2-[3-(furan-3-yl)-4-oxo-7-(pyrrolidin-1-yl)-1,4-dihydroquinolin-1-yl]-N-[2-(trifluoromethyl)phenyl]acetamide (1 g, 79%) as an yellow solid. LCMS (ESI+): m/z 482.0 [M+H]+

### Synthesis of Compound 85

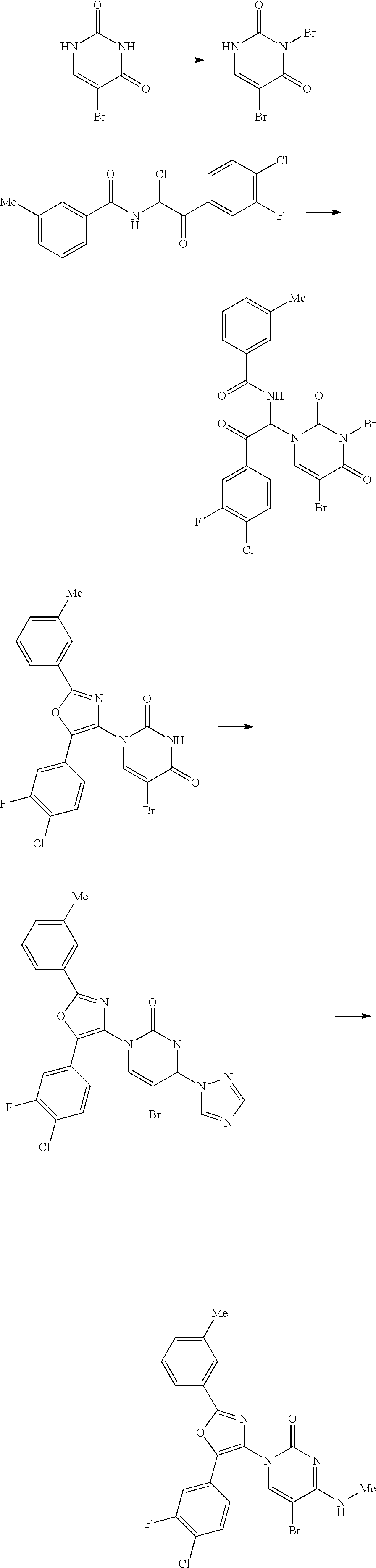

### 3-benzoyl-5-bromopyrimidine-2,4(1H,3H)-dione

A mixture of 5-bromo-1,2,3,4-tetrahydropyrimidine-2,4-dione (10 g, 52.3 mmol) and benzoyl chloride (18.2 g, 130 mmol) in mixed solvent of acetonitrile (100 mL) and pyridine (50 mL) was stirred at room temperature for 4 days. The reaction mixture was evaporated in vacuo. The residue was suspended into 1,4-dioxane (200 mL) and treated with 0.25M K₂CO₃ solution (100 mL) and stirred for 16 hour. The mixture was evaporated in order to remove 1,4-dioxane, and then water (100 mL) was added into the mixture. The precipitate was collected on a paper filter. The obtained crude solid was suspended into ethanol and stirred at 50° C. for 3 hour. The precipitate was collected on a paper filter to give 3-benzoyl-5-bromopyrimidine-2,4(1H,3H)-dione (12.0 g, 78%) as a white solid.

¹H NMR (300 MHz, DMSO-d6) δ 12.04 (s, 1H), 8.19 (s, 1H), 8.09-7.97 (m, 2H), 7.86-7.70 (m, 1H), 7.70-7.54 (m, 2H).

### N-(1-(3-benzoyl-5-bromo-2,4-dioxo-3,4-dihydropyrimidin-1(2H)-yl)-2-(4-chloro-3-fluorophenyl)-2-oxoethyl)-3-methylbenzamide

3-Benzoyl-5-bromopyrimidine-2,4(1H,3H)-dione (1.0 eq), (N-(1-chloro-2-(4-chloro-3-fluorophenyl)-2-oxoethyl)-3-methylbenzamide (1.0 eq), and triphenylphosphine (1.1 eq) were placed in a flask and suspended in dry toluene. The toluene was evaporated under high vacuum. This process was repeated 3 times. This mixture was then dissolved in anhydrous THF (10.0 vol) and di-tert-butyl azodicarboxylate (1.3 eq) was added in one portion. The resulting mixture was stirred at room temperature for 16 hours. The solvent was removed under reduced pressure. The crude material was dissolved in methanol and put into an ice bath. The resulting formed precipitate was collected by vacuum filtration, washed with methanol and hexanes, and dried under vacuum to give N-[1-(3-benzoyl-5-bromo-2,4-dioxo-1,2,3,4-tetrahydropyrimidin-1-yl)-2-(4-chloro-3-fluorophenyl)-2-oxoethyl]-3-methylbenzamide.

¹H NMR (300 MHz, DMSO-d6) δ 9.98 (d, J=8.4 Hz, 1H), 8.44 (s, 1H), 8.02-7.93 (m, 2H), 7.93-7.76 (m, 4H), 7.76-7.53 (m, 5H), 7.50-7.34 (m, 2H), 2.36 (s, 3H).

### 3-benzoyl-5-bromo-1-[5-(4-chloro-3-fluorophenyl)-2-(3-methylphenyl)-1,3-oxazol-4-yl]-1,2,3,4-tetrahydropyrimidine-2,4-dione

N-[1-(3-Benzoyl-5-bromo-2,4-dioxo-1,2,3,4-tetrahydropyrimidin-1-yl)-2-(4-chloro-3-fluorophenyl)-2-oxoethyl]-3-methylbenzamide (1.0 eq), hexachloroethane (2.0 eq), and triphenylphosphine (2.0 eq) were dissolved in anhydrous MeCN (20.0 vol). After stirring for 10 minutes at room temperature, pyridine (4.0 eq) was added, and the reaction mixture was stirred at 60° C. for 90 minutes. Upon cooling to room temperature, the formed precipitate was collected by vacuum filtration, washed with methanol, and dried under vacuum to give 3-benzoyl-5-bromo-1-[5-(4-chloro-3-fluorophenyl)-2-(3-methylphenyl)-1,3-oxazol-4-yl]-1,2,3,4-tetrahydropyrimidine-2,4-dione.

¹H NMR (300 MHz, DMSO-d6) δ 8.71 (s, 1H), 8.31-8.13 (m, 2H), 8.06-7.91 (m, 3H), 7.90-7.79 (m, 1H), 7.79-7.58 (m, 4H), 7.57-7.40 (m, 2H), 2.43 (s, 3H).

### 5-bromo-1-[5-(4-chloro-3-fluorophenyl)-2-(3-methylphenyl)-1,3-oxazol-4-yl]-4-(1H-1,2,4-triazol-1-yl)-1,2-dihydropyrimidin-2-one

3-Benzoyl-5-bromo-1-[5-(4-chloro-3-fluorophenyl)-2-(3-methylphenyl)-1,3-oxazol-4-yl]-1,2,3,4-tetrahydropyrimidine-2,4-dione (1.0 eq) was suspended in a mixture of anhydrous MeCN (20.0 vol) and anhydrous DCM (5.0 vol). Then, triethylamine (12.0 eq) and 1,2,4-triazole (8.0 eq) were added followed by the dropwise addition of phosphorus(V) oxychloride (2.0 eq). The reaction mixture was stirred at room temperature for 16 hours. The formed precipitate was collected by vacuum filtration, washed with MeCN, and dried under vacuum to afford 5-bromo-1-[5-(4-chloro-3-fluorophenyl)-2-(3-methylphenyl)-1,3-oxazol-4-yl]-4-(1H-1,2,4-triazol-1-yl)-1,2-dihydropyrimidin-2-one.

¹H NMR (300 MHz, DMSO-d6) δ 9.42 (s, 1H), 9.10 (s, 1H), 8.49 (s, 1H), 8.05 (s, 1H), 8.00 (d, J=7.5 Hz, 1H), 7.91 (dd, J=10.4, 2.1 Hz, 1H), 7.72 (t, J=8.1 Hz, 1H), 7.59-7.43 (m, 3H), 2.45 (s, 3H).

### 5-bromo-1-(5-(4-chloro-3-fluorophenyl)-2-(m-tolyl)oxazol-4-yl)-4-(methylamino)pyrimidin-2(1H)-one. Compound 85

To a suspension of 5-bromo-1-[5-(4-chloro-3-fluorophenyl)-2-(3-methylphenyl)-1,3-oxazol-4-yl]-4-(1H-1,2,4-triazol-1-yl)-1,2-dihydropyrimidin-2-one (1.0 eq) in anhydrous MeOH (20.0 vol) was added methylamine solution (2M in THF, 1.5 eq). The reaction mixture was stirred at room temperature for 16 hours. The formed precipitate was collected by vacuum filtration and washed with methanol and acetonitrile. The solid was dried under vacuum to afford 5-bromo-1-(5-(4-chloro-3-fluorophenyl)-2-(m-tolyl)oxazol-4-yl)-4-(methylamino)pyrimidin-2(1H)-one. [M+H]⁺ 451.1 m/z.

### Synthesis of Compound 150

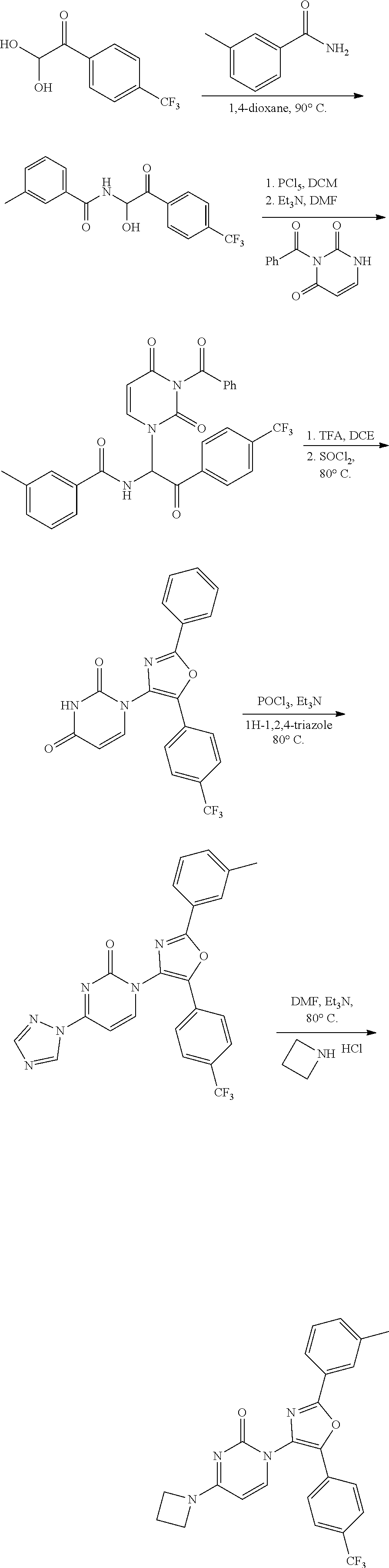

### N-(1-hydroxy-2-oxo-2-(4-(trifluoromethyl)phenyl)ethyl)-3-methylbenzamide

N-(1-Hydroxy-2-oxo-2-(4-(trifluoromethyl)phenyl)ethyl)-3-methylbenzamide was prepared similarly to the preparation of 3-chloro-N-(2-(4-chloro-3-fluorophenyl)-1-hydroxy-2-oxoethyl)benzamide.

LCMS: rt 2.28 min. [M+H]⁺ 360.0 m/z.

### N-(1-(3-benzoyl-2,4-dioxo-3,4-dihydropyrimidin-1(2H)-yl)-2-oxo-2-(4-(trifluoromethyl)phenyl)ethyl)-3-methylbenzamide

N-(1-(3-Benzoyl-2,4-dioxo-3,4-dihydropyrimidin-1(2H)-yl)-2-oxo-2-(4-(trifluoromethyl)phenyl)ethyl)-3-methylbenzamide was prepared similarly to the preparation of 3-chloro-N-(2-(4-chloro-3-fluorophenyl)-1-(5-fluoro-4-(methylamino)-2-oxopyrimidin-1(2H)-yl)-2-oxoethyl)benzamide.

LCMS: rt 2.26 min. [M+Na]⁺ 558.0 m/z.

### 1-(2-(m-tolyl)-5-(4-(trifluoromethyl)phenyl)oxazol-4-yl)pyrimidine-2,4(1H,3H)-dione

To a solution of N-(1-(3-benzoyl-2,4-dioxo-3,4-dihydropyrimidin-1(2H)-yl)-2-oxo-2-(4-(trifluoromethyl)phenyl)ethyl)-3-methylbenzamide (0.500 g, 1 Eq, 934 μmol) in DCE (5.0 mL) was added 2,2,2-trifluoroacetic acid (2.66 g, 25 Eq, 23.3 mmol), and the reaction mixture was heated to 60° C. 1 hour. The reaction was concentrated and dried under vacuum at 50° C. to give crude N-(1-(2,4-dioxo-3,4-dihydropyrimidin-1(2H)-yl)-2-oxo-2-(4-(trifluoromethyl)phenyl)ethyl)-3-methylbenzamide as brown solid. LCMS: rt 2.31 min. [M+H]⁺ 432 m/z. The crude residue was treated with thionyl chloride (4.9 g, 3.0 mL, 44 Eq, 41 mmol) and stirred at 60° C. for 1 hour. The reaction was then heated to 80° C. for 4 hours. The reaction was cooled to room temperature and concentrated. Ice water was added and a precipitate formed. The precipitate was collected by vacuum filtration and triturated with ether. This solid was collected by vacuum filtration and dried under vacuum to give 1-(2-(m-tolyl)-5-(4-(trifluoromethyl)phenyl)oxazol-4-yl)pyrimidine-2,4(1H,3H)-dione (95.4 mg, 231 μmol, 24.7%) as white solid.

LCMS: rt 2.63 min. [M+H]⁺ 414.0 m/z.

### 1-(2-(m-tolyl)-5-(4-(trifluoromethyl)phenyl)oxazol-4-yl)-4-(1H-1,2,4-triazol-1-yl)pyrimidin-2(1H)-one

1-(2-(m-Tolyl)-5-(4-(trifluoromethyl)phenyl)oxazol-4-yl)-4-(1H-1,2,4-triazol-1-yl)pyrimidin-2(1H)-one was prepared similarly to the preparation of 1-(5-(4-chloro-3-fluorophenyl)-2-(m-tolyl)oxazol-4-yl)-5-fluoro-4-(1H-1,2,4-triazol-1-yl)pyrimidin-2(1H)-one.

LCMS: rt 2.74 min. [M+H]⁺ 465.0 m/z.

### 4-(azetidin-1-yl)-1-(2-(m-tolyl)-5-(4-(trifluoromethyl)phenyl)oxazol-4-yl)pyrimidin-2(1H)-one. Compound 150

To a slurry of 1-(2-(m-tolyl)-5-(4-(trifluoromethyl)phenyl)oxazol-4-yl)-4-(1H-1,2,4-triazol-1-yl)pyrimidin-2(1H)-one (30.0 mg, 1 Eq, 64.6 μmol) in DMF (1 mL) was added triethylamine (32.7 mg, 45.0 μL, 5 Eq, 323 μmol) followed by azetidine hydrochloride (21.8 mg, 97 Wt %, 3.5 Eq, 226 μmol). The resulting brown slurry was stirred 50° C. for 1.5 hours. The reaction was cooled to room temperature and treated with H₂O. The resulting precipitate was collected by vacuum filtration, washed with water, and dried under vacuum to give 4-(azetidin-1-yl)-1-(2-(m-tolyl)-5-(4-(trifluoromethyl)phenyl)oxazol-4-yl)pyrimidin-2(1H)-one (23.4 mg, 51.7 μmol, 80.1%) as light brown solid. ¹H NMR (400 MHz, DMSO-d₆) d ppm 7.98 (s, 1H), 7.93 (d, J=7.82 Hz, 1H), 7.83-7.91 (m, 3H), 7.79 (d, J=8.07 Hz, 2H), 7.47-7.53 (m, 1H), 7.41-7.46 (m, 1H), 5.96 (d, J=7.34 Hz, 1H), 4.23 (t, J=7.58 Hz, 2H), 4.14 (t, J=7.58 Hz, 2H), 2.43 (s, 3H), 2.35-2.40 (m, 2H). ¹⁹F NMR (376 MHz, DMSO-d₆) d ppm −61.18 (s, 3F). LCMS: rt 2.56 min. [M+H]⁺ 453.0 m/z.

Additional Compounds Prepared in an Analogous Manner to Compound 150:

| | Exact mass | | |
|---|---|---|---|
| Compound | (g/mol) | Found | Assignment |
| 151 | 469.1117 | 470.1 | [M + H]+ |
| 152 | 422.0946 | 423.0 | [M + H]+ |
| 153 | 440.0852 | 441.0 | [M + H]+ |
| 154 | 497.143 | 498.0 | [M + H]+ |
| 158 | 502.102 | 503.12 | [M + H]+ |
| 159 | 432.1353 | 433.09 | [M + H]+ |
| 160 | 472.0914 | 473.01 | [M + H]+ |
| 161 | 478.1617 | 479.16 | [M + H]+ |
| 162 | 480.1364 | 481.17 | [M + H]+ |
| 163 | 480.1409 | 481.2 | [M + H]+ |
| 164 | 486.107 | 487.12 | [M + H]+ |
| 165 | 486.107 | 487.03 | [M + H]+ |
| 166 | 486.107 | 487.1 | [M + H]+ |
| 167 | 486.107 | 487.16 | [M + H]+ |
| 168 | 488.1272 | 489.0 | [M + H]+ |
| 169 | 500.1227 | 501.17 | [M + H]+ |
| 170 | 500.1227 | 501.15 | [M + H]+ |
| 171 | 504.0976 | 505.08 | [M + H]+ |
| 172 | 516.1176 | 517.05 | [M + H]+ |
| 173 | 518.1133 | 519.25 | [M + H]+ |
| 174 | 534.1082 | 535.28 | [M + H]+ |

### Synthesis of 1-(5-(4-chloro-3-fluorophenyl)oxazol-4-yl)-4-(1H-1,2,4-triazol-1-yl)pyrimidin-2(1H)-one

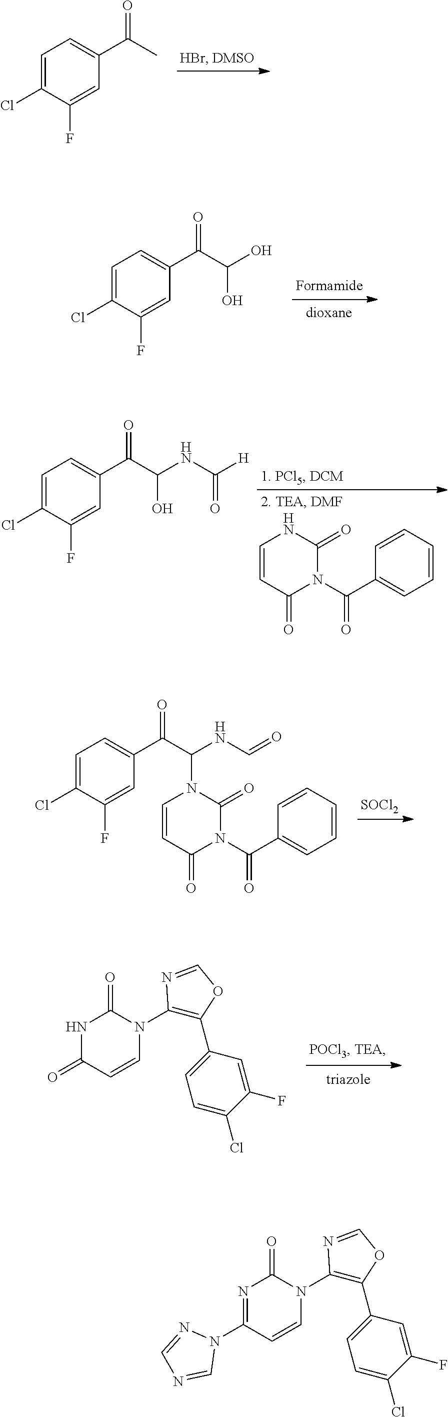

### 1-(4-chloro-3-fluorophenyl)-2,2-dihydroxyethan-1-one

To a stirred solution of 1-(4-chloro-3-fluorophenyl)ethan-1-one (20.0 g, 1.0 Eq, 116 mmol) in DMSO (100 mL) was added HBr (58.6 g, 39.3 mL, 48 Wt % in water, 3.0 Eq, 348 mmol), and the mixture was stirred at room temperature for 1 hour. The mixture was then stirred at 50° C. for 16 hours. The reaction mixture was poured into ice water and a precipitate formed. This solid was collected by vacuum filtration and washed with hexanes to afford 1-(4-chloro-3-fluorophenyl)-2,2-dihydroxyethan-1-one (14.0 g, 68.4 mmol, 59.1%) as an off-white solid.

¹H NMR (400 MHz, DMSO-d6) δ ppm 8.00 (dd, 1H, J=1.8, 10.1 Hz), 7.93 (td, 1H, J=1.0, 8.4 Hz), 7.78 (dd, 1H, J=7.6, 8.2 Hz), 6.98 (d, 2H, J=5.5 Hz), 5.61 (bs, 1H).

### N-(2-(4-chloro-3-fluorophenyl)-1-hydroxy-2-oxoethyl)formamide

To a stirred solution of 1-(4-chloro-3-fluorophenyl)-2,2-dihydroxyethan-1-one (5.0 g, 1 Eq, 24 mmol) in dioxane (50.0 mL) was added formamide (5.5 g, 4.9 mL, 5.0 Eq, 0.12 mol) at room temperature under an argon atmosphere. The resulting reaction mixture was stirred at 90° C. for 3 hours. After cooling to room temperature, the solvent was evaporated under reduced pressure to afford crude compound as a pale-yellow liquid. The crude compound was triturated with MTBE and hexanes. The precipitated solid was collected by vacuum filtration and dried under vacuum to afford N-(2-(4-chloro-3-fluorophenyl)-1-hydroxy-2-oxoethyl)formamide (3.0 g, 13 mmol, 53%) as an off-white solid.

¹H NMR (400 MHz, DMSO-d6) δ ppm 9.00 (d, J=8.4 Hz, 1H), 8.09 (dd, J=0.7, 1.6 Hz, 1H), 7.92 (dd, J=1.4, 10.1 Hz, 1H), 7.83-7.77 (m, 2H), 6.86 (d, J=7.2 Hz, 1H), 6.31 (t, J=8.0 Hz, 1H).

### N-(1-(3-benzoyl-2,4-dioxo-3,4-dihydropyrimidin-1(2H)-yl)-2-(4-chloro-3-fluorophenyl)-2-oxoethyl)formamide

To a stirred solution of N-(2-(4-chloro-3-fluorophenyl)-1-hydroxy-2-oxoethyl)formamide (12.0 g, 1 Eq, 51.8 mmol) in DCM (120 mL) was added PCl₅ (12.9 g, 1.2 Eq, 62.2 mmol) at room temperature portion wise. The resulting reaction mixture was stirred for 2 h. The reaction mixture was concentrated under reduced pressure to afford crude chloro compound. This material was then triturated with hexanes (100 mL), collected by vacuum filtration, and dried under vacuum to afford the chloro intermediate which was used directly. In a separate 3-neck round-bottomed flask under nitrogen atmosphere, were combined 3-benzoylpyrimidine-2,4(1H,3i)-dione (11.2 g, 1.0 Eq, 51.8 mmol), DMF (100 mL), and TEA (15.7 g, 21.7 mL, 3.0 Eq, 155 mmol). Then, the chloro intermediate form above was added dropwise as a solution in DMF (100 mL). The resulting reaction mixture was stirred at room temperature for 4 hours. The reaction was concentrated under vacuum and diluted with ice water. The resulting precipitate was collected by vacuum filtration, washed with saturated sodium bicarbonate, and dried under vacuum to afford crude compound. The crude material was adsorbed on silica gel and purified by silica gel column chromatography (40-65% EtOAc in petroleum ether) to give N-(1-(3-benzoyl-2,4-dioxo-3,4-dihydropyrimidin-1(2H)-yl)-2-(4-chloro-3-fluorophenyl)-2-oxoethyl)formamide (14.0 g, 32.4 mmol, 62.5%) as a yellow sticky solid.

¹H NMR (400 MHz, DMSO-d6) δ ppm 9.64 (d, 1H, J=7.6 Hz), 8.26 (s, 1H), 8.06 (d, 1H, J=8.2 Hz), 7.8-7.9 (m, 3H), 7.69 (dd, 1H, J=1.6, 8.4 Hz), 7.61 (d, 2H, J=2.1 Hz), 7.5-7.5 (m, 2H), 7.29 (d, 1H, J=8.0 Hz), 6.05 (d, 1H, J=8.2 Hz).

### 1-(5-(4-chloro-3-fluorophenyl)oxazol-4-yl)pyrimidine-2,4(1H,3H)-dione

A stirred solution of N-(1-(3-benzoyl-2,4-dioxo-3,4-dihydropyrimidin-1(2H)-yl)-2-(4-chloro-3-fluorophenyl)-2-oxoethyl)formamide (14 g, 1 Eq, 33 mmol) in thionyl chloride (22.8 g, 14.0 mL, 5.9 Eq, 192 mmol) was heated at 65° C. for 32 hours in a sealed tube. Upon cooling to room temperature, the reaction mixture was concentrated under vacuum, diluted with saturated aqueous sodium bicarbonate, and extracted with 30% IPA in chloroform (3×15 ml). The combined organic layers were dried over sodium sulfate and concentrated under vacuum to afford crude compound. The crude compound was adsorbed onto silica gel and purified by silica gel column chromatography (4-10% methanol in EtOAc) to afford still impure material. This material was then triturated with MTBE to afford 1-(5-(4-chloro-3-fluorophenyl)oxazol-4-yl)pyrimidine-2,4(1H,3H)-dione (5.0 g, 16 mmol, 50%) as an off-white solid.

¹H NMR (400 MHz, DMSO-d6) δ ppm 11.70 (s, 1H), 8.69 (s, 1H), 7.7-7.8 (m, 2H), 7.58 (dd, 1H, J=2.0, 10.2 Hz), 7.36 (ddd, 1H, J=0.8, 2.0, 8.5 Hz), 5.84 (dd, 1H, J=2.2, 8.0 Hz)

### 1-(5-(4-chloro-3-fluorophenyl)oxazol-4-yl)-4-(1H-1,2,4-triazol-1-yl)pyrimidin-2(1H)-one

To a stirred solution of 1-(5-(4-chloro-3-fluorophenyl)oxazol-4-yl)pyrimidine-2,4(1H,3H)-dione (5.0 g, 1 Eq, 16 mmol) in MeCN (100 mL) were added 1H-1,2,4-triazole (9.0 g, 8 Eq, 0.13 mol) and triethylamine (20 g, 27 mL, 12 Eq, 0.20 mol). Then, phosphoryl trichloride (5.0 g, 3.0 mL, 2.0 Eq, 33 mmol) was added dropwise under nitrogen at 0° C., and the reaction mixture was stirred at 0° C. for 1 hour. A solid precipitate formed during the course of the reaction, and it was collected by vacuum filtration. This material was then washed with 50% MeCN in water (100 mL), water (100 mL), and MeCN (100 mL). The solid was then dried under vacuum. The material was then triturated with EtOAc and the solid was collected by vacuum filtration. After drying under vacuum, 1-(5-(4-chloro-3-fluorophenyl)oxazol-4-yl)-4-(1H-1,2,4-triazol-1-yl)pyrimidin-2(1H)-one (3.6 g, 9.2 mmol, 57%) was obtained as an off-white solid.

¹H NMR (400 MHz, DMSO-d6) δ ppm 9.55 (1H, s), 8.77 (1H, s), 8.56 (1H, d, J=7.2 Hz), 8.49 (1H, s), 7.71 (1H, t, J=8.1 Hz), 7.60 (1H, dd, J=10.2 Hz, 2.0 Hz), 7.36 (1H, ddd, J=8.4 Hz, 2.0 Hz, 0.7 Hz), 7.20 (1H, d, J=7.2 Hz).

### Synthesis of Compound 155.

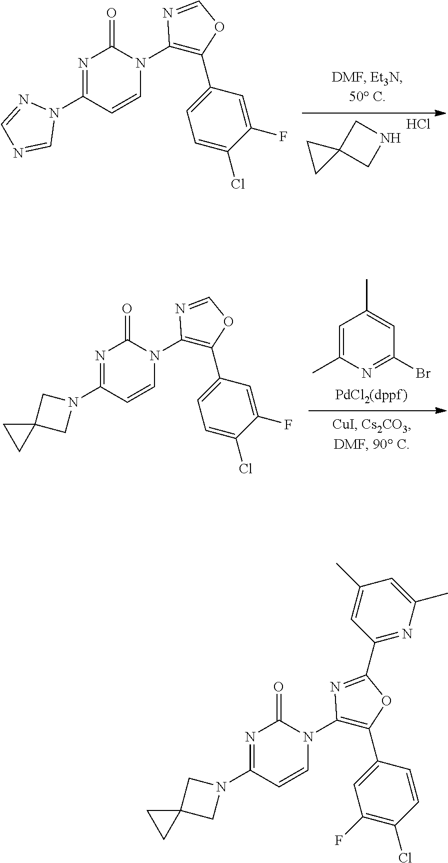

### 1-(5-(4-chloro-3-fluorophenyl)oxazol-4-yl)-4-(5-azaspiro[2.3]hexan-5-yl)pyrimidin-2(1H)-one

1-(5-(4-Chloro-3-fluorophenyl)oxazol-4-yl)-4-(5-azaspiro[2.3]hexan-5-yl)pyrimidin-2(1H)-one was prepared similarly from 1-(5-(4-chloro-3-fluorophenyl)oxazol-4-yl)-4-(1H-1,2,4-triazol-1-yl)pyrimidin-2(1H)-one to the preparation of 4-(azetidin-1-yl)-1-(2-(m-tolyl)-5-(4-(trifluoromethyl)phenyl)oxazol-4-yl)pyrimidin-2(1H)-one.

LCMS: rt 2.00 min. [M+H]⁺ 373.1 m/z.

### 1-(5-(4-chloro-3-fluorophenyl)-2-(4,6-dimethylpyridin-2-yl)oxazol-4-yl)-4-(5-azaspiro[2.3]hexan-5-yl)pyrimidin-2(1H)-one. Compound 155

1-(5-(4-Chloro-3-fluorophenyl)-2-(4,6-dimethylpyridin-2-yl)oxazol-4-yl)-4-(5-azaspiro[2.3]hexan-5-yl)pyrimidin-2(1H)-one was prepared similarly to the preparation of 1-(5-(4-chloro-3-fluorophenyl)-2-(4,6-dimethylpyridin-2-yl)oxazol-4-yl)-4-(3-fluoro-3-methylazetidin-1-yl)pyrimidin-2(1H)-one.

¹H NMR (400 MHz, DMSO-d₆) δ 7.94 (s, 1H), 7.86 (d, J=7.4 Hz, 1H), 7.79 (t, J=8.1 Hz, 1H), 7.62-7.54 (m, 1H), 7.40-7.35 (m, 1H), 7.31 (s, 1H), 5.96 (d, J=7.4 Hz, 1H), 4.28 (s, 2H), 4.18 (s, 2H), 2.55 (s, 3H), 2.41 (s, 3H), 0.74 (s, 4H).

LCMS: rt 2.29 min. [M+H]⁺ 478.0 m/z.

### Synthesis of Compound 156.

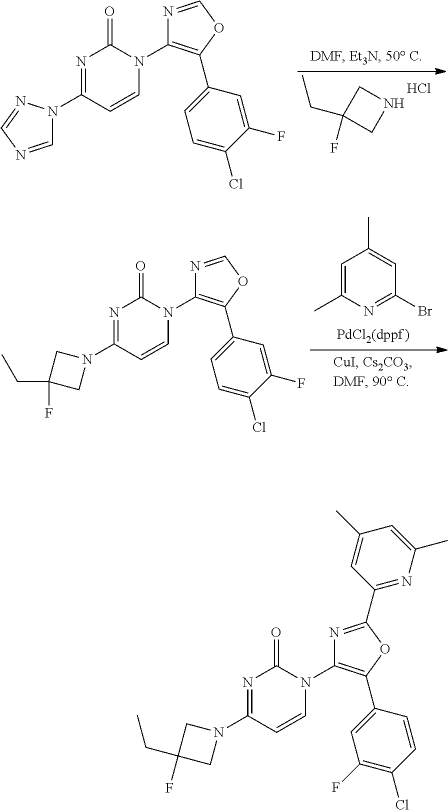

### 1-(5-(4-chloro-3-fluorophenyl)oxazol-4-yl)-4-(3-ethyl-3-fluoroazetidin-1-yl)pyrimidin-2(1H)-one

1-(5-(4-Chloro-3-fluorophenyl)oxazol-4-yl)-4-(3-ethyl-3-fluoroazetidin-1-yl)pyrimidin-2(1H)-one was prepared similarly from 1-(5-(4-chloro-3-fluorophenyl)oxazol-4-yl)-4-(1H-1,2,4-triazol-1-yl)pyrimidin-2(1H)-one to the preparation of 4-(azetidin-1-yl)-1-(2-(m-tolyl)-5-(4-(trifluoromethyl)phenyl)oxazol-4-yl)pyrimidin-2(1H)-one.

LCMS: rt 2.61 min. [M+H]⁺ 393.0 m/z.

### 1-(5-(4-chloro-3-fluorophenyl)-2-(6-methylpyridin-2-yl)oxazol-4-yl)-4-(3-ethyl-3-fluoroazetidin-1-yl)pyrimidin-2(1H)-one. Compound 156

1-(5-(4-Chloro-3-fluorophenyl)-2-(4,6-dimethylpyridin-2-yl)oxazol-4-yl)-4-(3-ethyl-3-fluoroazetidin-1-yl)pyrimidin-2(1H)-one was prepared similarly to the preparation of 1-(5-(4-chloro-3-fluorophenyl)-2-(4,6-dimethylpyridin-2-yl)oxazol-4-yl)-4-(3-fluoro-3-methylazetidin-1-yl)pyrimidin-2(1H)-one.

¹H NMR (400 MHz, DMSO-d₆) d ppm 7.88-7.97 (m, 2H), 7.77 (t, J=8.31 Hz, 1H), 7.57 (d, J=10.27 Hz, 1H), 7.38 (d, J=8.31 Hz, 1H), 7.31 (s, 1H), 6.03 (d, J=7.34 Hz, 1H), 4.15-4.40 (m, 4H), 2.54 (s, 3H), 2.40 (s, 3H), 1.89-2.04 (m, 2H), 0.96 (t, J=7.21 Hz, 3H).

LCMS: rt 2.21 min. [M+H]⁺ 498.2 m/z.

### Synthesis of Compound 157.

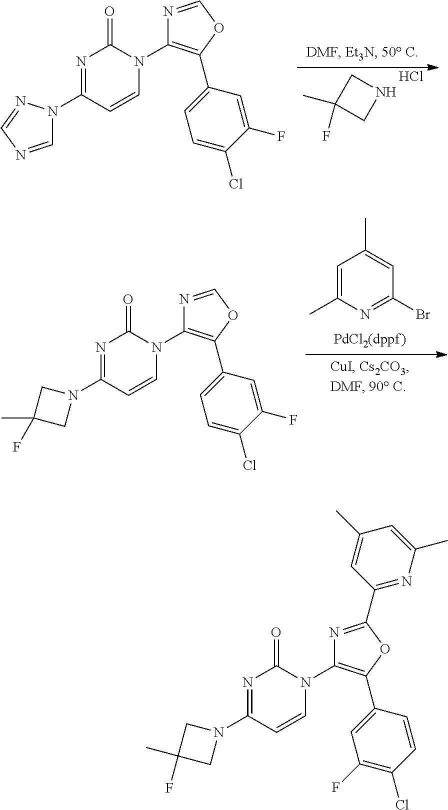

### 1-(5-(4-chloro-3-fluorophenyl)oxazol-4-yl)-4-(3-fluoro-3-methylazetidin-1-yl)pyrimidin-2(1H)-one

1-(5-(4-Chloro-3-fluorophenyl)oxazol-4-yl)-4-(3-fluoro-3-methylazetidin-1-yl)pyrimidin-2(1H)-one was prepared from 1-(5-(4-chloro-3-fluorophenyl)oxazol-4-yl)-4-(1H-1,2,4-triazol-1-yl)pyrimidin-2(1H)-one similarly to the preparation of 4-(azetidin-1-yl)-1-(2-(m-tolyl)-5-(4-(trifluoromethyl)phenyl)oxazol-4-yl)pyrimidin-2(1H)-one.

LCMS: rt 1.81 min. [M+H]⁺ 379.0 m/z.

### 1-(5-(4-chloro-3-fluorophenyl)-2-(4,6-dimethylpyridin-2-yl)oxazol-4-yl)-4-(3-fluoro-3-methylazetidin-1-yl)pyrimidin-2(1H)-one. Compound 157

1-(5-(4-Chloro-3-fluorophenyl)oxazol-4-yl)-4-(3-fluoro-3-methylazetidin-1-yl)pyrimidin-2(1H)-one (75.0 mg, 1 Eq, 198 μmol), 2-bromo-4,6-dimethyl-pyridine (55.3 mg, 1.5 Eq, 297 μmol), cesium carbonate (194 mg, 3 Eq, 594 μmol), 1,1′-bis(diphenylphosphino)ferrocene-palladium(II) dichloride (21.7 mg, 0.15 Eq, 29.7 μmol), and copper(I) iodide (2.83 mg, 0.075 Eq, 14.9 μmol) were combined in DMF (3.0 mL). The reaction was then purged and refilled with argon 3 times and stirred at 90° C. for 16 hour. The reaction was let cool to room temperature and filtered through a pad of celite. The celite was further eluted with DCM, and the combined organic solution was concentrated to dryness. The residue was purified by flash column chromatography (SiO₂, 40 g, 0-10% MeOH in DCM) to give 1-(5-(4-chloro-3-fluorophenyl)-2-(4,6-dimethylpyridin-2-yl)oxazol-4-yl)-4-(3-fluoro-3-methylazetidin-1-yl)pyrimidin-2(1H)-one (23 mg, 47.5 μmol, 24% yield).

¹H NMR (400 MHz, DMSO-d₆) d ppm 7.93 (s, 1H), 7.89 (d, J=7.34 Hz, 1H), 7.77 (t, J=8.30 Hz, 1H), 7.55 (d, J=10.50 Hz, 1H), 7.37 (d, J=8.60 Hz, 1H), 7.31 (s, 1H), 5.99 (d, J=7.34 Hz, 1H), 4.28-4.41 (m, 2H), 4.21 (d, J=19.60 Hz, 2H), 2.54 (s, 3H), 2.40 (s, 3H), 1.67 (d, J=22.30 Hz, 3H).

¹⁹F NMR (376 MHz, DMSO-d₆) d ppm −114.69 (s, 1F), −139.40-−139.05 (m, 1F).

LCMS: rt 2.11 min. [M+H]⁺ 484.1 m/z.

### Synthesis of Compound 175.

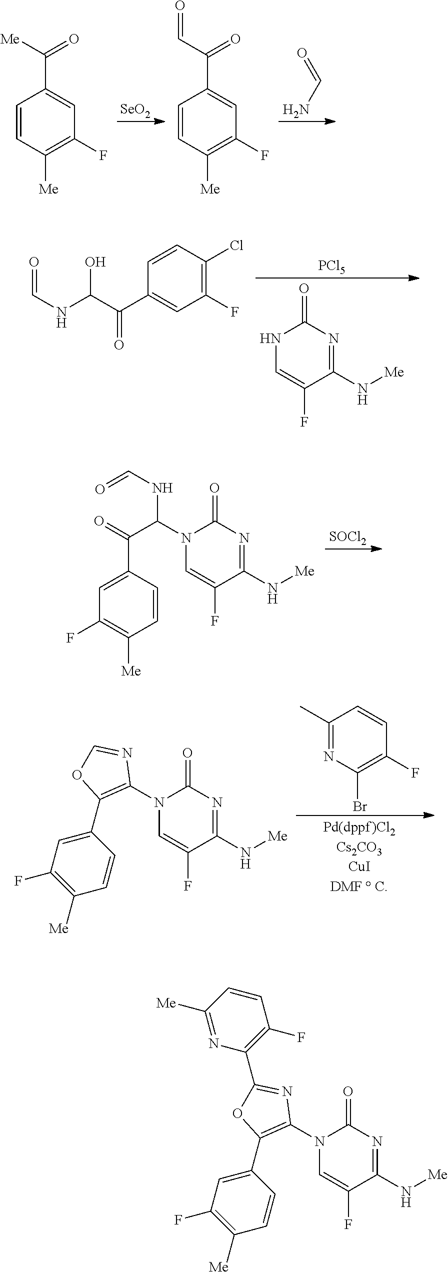

### 2-(3-fluoro-4-methylphenyl)-2-oxoacetaldehyde

Selenium dioxide (13.1 g, 1.8 Eq, 118 mmol) was combined with water (10.0 mL) and dioxane (100 mL) in a 200 mL medium pressure flask. The flask was heated to 70° C. until all material completely dissolved. To the reaction mixture at 70° C., was added 4-acetyl-2-fluorotoluene (10.0 g, 1 eq, 65.7 mmol), and the reaction flask was sealed and let stir at 100° C. for 16 hours. The reaction was cooled to room temperature, filtered through a pad of celite, and further eluted with EtOAc. The liquid filtrate was concentrated to a viscous brown oil, which was treated with H₂O (50 mL) and heated to 100° C. under a reflux condenser for 6 hours. Upon cooling to room temperature, the formed precipitate was collected by vacuum filtration, washed with cold water, and dried under vacuum to give 2-(3-fluoro-4-methylphenyl)-2-oxoacetaldehyde (10.5 g, 63.2 mmol, 96.2%) as white solid. This material was used directly in the next step without characterization.

### N-(2-(4-chloro-3-fluorophenyl)-1-hydroxy-2-oxoethyl)formamide

To a slurry of 2-(3-fluoro-4-methylphenyl)-2-oxoacetaldehyde (3.0 g, 1.1 eq, 16.3 mmol) in dioxane (20 mL) was added formamide (2 g, 1.77 mL, 3.0 eq, 48.9 mmol). The reaction mixture was heated to 90° C. for 16 hours. Upon cooling to room temperature, the reaction was concentrated and diluted with ethanol. The resulting solid was collected by vacuum filtration and dried in a vacuum oven to give N-(2-(3-fluoro-4-methylphenyl)-1-hydroxy-2-oxoethyl)formamide (1.52 g, 7.20 mmol, 48.6%). This material was used in the next step without purification or characterization.

### N-(1-(5-fluoro-4-(methylamino)-2-oxopyrimidin-1(2H)-yl)-2-(3-fluoro-4-methylphenyl)-2-oxoethyl)formamide

To a slurry of N-(2-(3-fluoro-4-methylphenyl)-1-hydroxy-2-oxoethyl)formamide (2.34 g, 1 eq, 10.4 mmol) in DCM (70 mL) was added phosphorus pentachloride (2.27 g, 1.05 Eq, 10.9 mmol). The resulting homogeneous solution was stirred at 65° C. for 2 hours. After cooling to room temperature, the reaction was concentrated and dried under vacuum to give the intermediate chloro-adduct as yellow solid. To a solution of 4-(methylamino)pyrimidin-2(1H)-one (1.43 g, 1.1 Eq, 11.4 mmol) in DMF (5 mL) was added triethylamine (3.15 g, 4.3 mL, 3 Eq, 31.2 mmol), and the mixture was stirred at room temperature for 15 minutes. The reaction mixture was chilled to 0° C., and the intermediate chloro-adduct was added portion-wise. The cold bath was removed, and the resulting reaction mixture was stirred at ambient temperature for 30 minutes. Water (40 mL) was then added, and the resulting precipitate was collected by vacuum filtration, washed with cold water, and dried under vacuum at 50° C. to afford N-(1-(5-fluoro-4-(methylamino)-2-oxopyrimidin-1(2H)-yl)-2-(3-fluoro-4-methylphenyl)-2-oxoethyl)formamide (1.83 g, 5.47 mmol, 52.7%).

### 5-fluoro-1-(5-(3-fluoro-4-methylphenyl)oxazol-4-yl)-4-(methylamino)pyrimidin-2(1H)-one

N-(1-(5-Fluoro-4-(methylamino)-2-oxopyrimidin-1(2H)-yl)-2-(3-fluoro-4-methylphenyl)-2-oxoethyl)formamide (2.82 g, 1 Eq, 8.00 mmol) was treated with thionyl chloride (30 eq, 17.5 mL) and heated to 65° C. for 3 hours. The reaction was cooled to room temperature and concentrated to give a dark brown viscous oil. The oil was then triturated with ether and dried under vacuum to afford 5-fluoro-1-(5-(3-fluoro-4-methylphenyl)oxazol-4-yl)-4-(methylamino)pyrimidin-2(1H)-one (1.8 g, 5.7 mmol, 71%) as a beige solid.

LCMS (ESI+): m/z 319.1, [M+H]⁺. RT 1.83 min.

### 5-fluoro-1-(5-(3-fluoro-4-methylphenyl)-2-(3-fluoro-6-methylpyridin-2-yl)oxazol-4-yl)-4-(methylamino)pyrimidin-2(1H)-one. Compound 175

5-Fluoro-1-(5-(3-fluoro-4-methylphenyl)oxazol-4-yl)-4-(methylamino)pyrimidin-2(1H)-one (100 mg, 0.314 mmol, 1 eq), 2-bromo-3-fluoro-6-methylpyridine (3 eq, 179 mg, 0.942 mmol), and cesium carbonate (307 mg, 3 Eq, 0.943 mmol) were combined in DMF (1 mL). Then, copper(I) iodide (4.49 mg, 0.075 Eq, 23.6 μmol) and Pd(dppf)Cl₂ (34.5 mg, 0.15 Eq, 47.1 μmol) were added. The reaction vial was evacuated and refilled with argon and stirred at 90° C. for 24 hours. Upon cooling to room temperature, the reaction was diluted with DCM and filtered through celite. Water was added, and the organic fraction was separated, dried over magnesium sulfate, and concentrated. The residue was purified by silica gel column chromatography (0-20% MeOH in DCM with 0.1% ammonium hydroxide). The collected product was then recrystallized in MeOH and to give 5-fluoro-1-(5-(3-fluoro-4-methylphenyl)-2-(3-fluoro-6-methylpyridin-2-yl)oxazol-4-yl)-4-(methylamino)pyrimidin-2(1H)-one (5 mg, 0.01 mmol, 4%).

LCMS (ESI+): m/z 428.1, [M+H]+. RT 2.25 min.

¹H NMR: (400 MHz, DMSO-d6) δ 8.48-8.47 (m, 1H), 8.13 (d, J=6.4 Hz, 1H), 7.91 (t, J=8.8 Hz, 1H), 7.57 (dd, J=8.4 Hz, 3.2 Hz, 1H), 7.48 (t, J=8.0 Hz, 1H), 7.31 (d, J=8.0 Hz, 1H), 7.25 (d, J=10.8 Hz, 1H), 2.91 (d, J=4.8 Hz, 3H), 2.59 (s, 3H), 2.29 (s, 3H).

Additional Compounds Prepared in an Analogous Manner to Compound 175:

| | Exact mass | | |
|---|---|---|---|
| Compound | (g/mol) | Found | Assignment |
| 175 | 427.1256 | 428.1 | [M + H]+ |
| 176 | 429.0804 | 430.2 | [M + H]+ |
| 177 | 429.0804 | 430.3 | [M + H]+ |
| 178 | 429.0804 | 430.2 | [M + H]+ |
| 179 | 430.0757 | 431.1 | [M + H]+ |
| 180 | 445.0753 | 446.1 | [M + H]+ |
| 294 | 427.1256 | 428.1 | [M + H]+ |

### Synthesis of Compound 77.

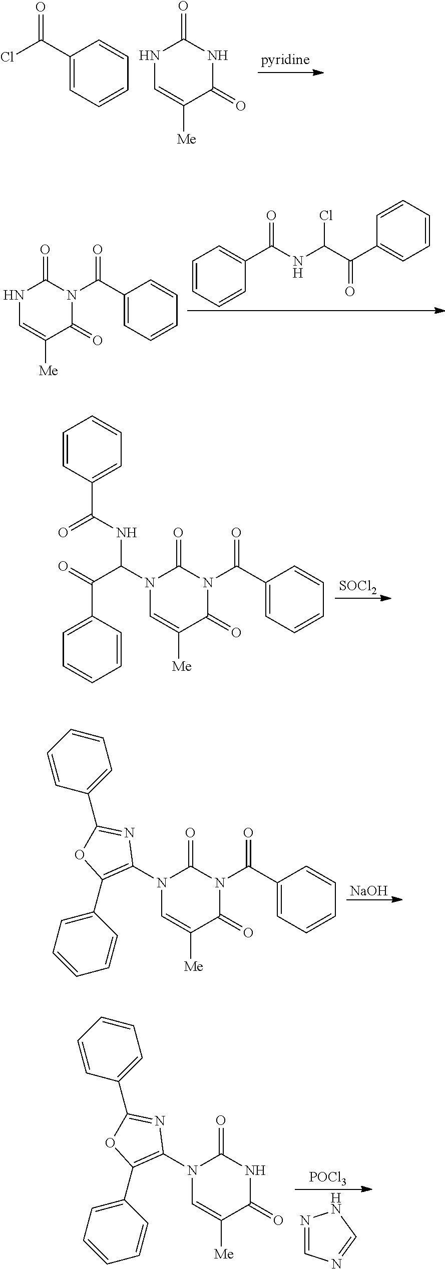

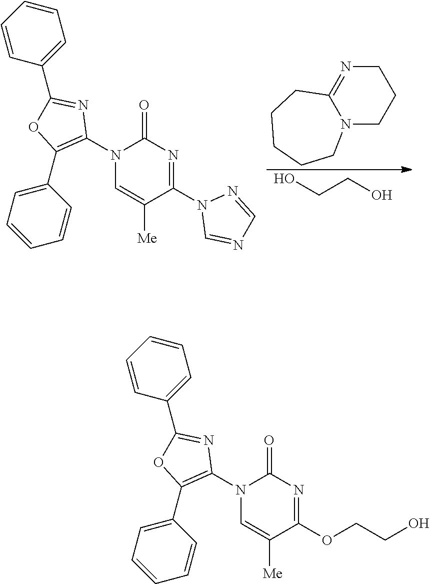

### 3-benzoyl-5-methylpyrimidine-2,4(1H,3H)-dione

To a mixture of 5-methylpyrimidine-2,4(1H,3H)-dione (10.0 g, 79.2 mmol) in pyridine (50 mL) was added benzoyl chloride (15.7 mL, 24.4 g, 134 mmol) at 0 deg. After stirring for 20 hr at room temperature, ice (75 g) was added at 0 deg. The mixture was stirred for 1 hr, and then at room temperature for 20 hr. The resulting mixture was diluted with water (200 mL), extracted with CH2Cl2 twice. The organic layer, which was cloudy, was washed with sat. NaHCO3 aq. three times (to get the almost clear solution) and dried over Na2SO4. The mixture was concentrated under reduced pressure. The residue was triturated with CH2Cl2, then heptane was added to get the solid suspended. The solid material obtained by filtration and washing with heptane was dried under reduced pressure at 45 deg. for 2 hr to give 3-benzoyl-5-methylpyrimidine-2,4(1H,3H)-dione (11.4 g, 62.6%) as a colorless solid. ¹H NMR (400 MHz, CDCl₃) δ 9.50 (s, 1H), 7.94 (d, 2H, J=8.4), 7.67 (t, 1H, J=7.6), 7.53-7.50 (m, 2H), 7.05-7.03 (m, 1H), 1.92 (s, 3H).

### N-(1-(3-benzoyl-5-methyl-2,4-dioxo-3,4-dihydropyrimidin-1(2H)-yl)-2-oxo-2-phenylethyl)benzamide

N-(1-chloro-2-oxo-2-phenylethyl)benzamide (1.30 g, 4.74 mmol) was added into mixture of 3-benzoyl-5-methylpyrimidine-2,4(1H,3H)-dione (1.0 g, 4.34 mmol) and sodium hydrogen carbonate (898 mg, 10.7 mmol) in N,N-dimethylformamide (25 mL) at 0° C. and stirred for 30 min at the same temperature. Then it was stirred at room temperature for 18 hour. The mixture was diluted with EtOAc and washed with water 2 times and brine, dried over Na₂SO₄ and evaporated in vacuo. The residue was purified by column chromatography (silica gel, Heptane:Ethyl acetate=1:1) to give N-(1-(3-benzoyl-5-methyl-2,4-dioxo-3,4-dihydropyrimidin-1(2H)-yl)-2-oxo-2-phenylethyl)benzamide (1.77 g, 87.6%) as a white solid. ¹H NMR (400 MHz, CDCl₃) δ 8.13 (d, 1H, J=6.4 Hz), 7.95-7.90 (m, 4H), 7.75-7.70 (m, 2H), 7.60-7.52 (m, 8H), 7.32-7.27 (m, 2H), 6.91 (d, 1H, J=6.8 Hz), 2.03 (s, 3H).

### 3-benzoyl-1-(2,5-diphenyloxazol-4-yl)-5-methylpyrimidine-2,4(1H,3H)-dione

N,N-dimethylformamide (828 mg, 11.3 mmol) was added dropwise into phosphoryl trichloride (3.47 g, 22.6 mmol) at 0° C. and stirred 10 min at the same temperature. The mixture was warmed to room temperature and a solution of N-(1-(3-benzoyl-5-methyl-2,4-dioxo-3,4-dihydropyrimidin-1(2H)-yl)-2-oxo-2-phenylethyl)benzamide (1.77 g, 3.78 mmol) in CH2Cl2 (2 mL) was added and stirred at 110° C. for 3 hour. Lots of precipitate generated and the mixture got unable to be stirred. 1,4-dioxane (20 mL) was added there, and resulting precipitate was collected on filter paper and washed with water to give 3-benzoyl-1-(2,5-diphenyloxazol-4-yl)-5-methylpyrimidine-2,4(1H,3H)-dione (372 mg, 22.0%) as a white solid. ¹H NMR (400 MHz, CDCl₃) δ 8.11-8.10 (m, 2H), 8.09-7.98 (m, 2H), 7.70-7.62 (m, 3H), 7.52-7.27 (m, 9H), 2.05 (s, 3H).

### 1-(2,5-diphenyloxazol-4-yl)-5-methylpyrimidine-2,4(1H,3H)-dione

A suspension of 3-benzoyl-1-(2,5-diphenyloxazol-4-yl)-5-methylpyrimidine-2,4(1H,3H)-dione (372 mg, 0.8276 mmol) in tetrahydrofuran (16 mL) and methanol (4 mL) was treated with 1 ml of 6N NaOH solution at room temperature for hour. Precipitate was collected on a paper filter to obtain 1-(2,5-diphenyloxazol-4-yl)-5-methylpyrimidine-2,4(1H,3H)-dione (46.0 mg, 16.1%) as a white solid.

¹H NMR (400 MHz, CDCl₃) δ 8.17 (d, 2H, J=7.6 Hz), 8.16-7.93 (m, 3H), 7.72-7.70 (m, 1H), 7.62-7.54 (m, 5H), 7.39 (d, 1H, J=9.2 Hz), 1.56 (s, 3H).

### 1-(2,5-diphenyloxazol-4-yl)-5-methyl-4-(1H-1,2,4-triazol-1-yl)pyrimidin-2(1H)-one

A mixture of 1-(2,5-diphenyloxazol-4-yl)-5-methylpyrimidine-2,4(1H,3H)-dione (40 mg, 0.1158 mmol), triethylamine (138 mg, 1.37 mmol) and 1H-1,2,4-triazole (63.8 mg, 0.9251 mmol) in acetonitrile (1 mL) was treated with phosphoryl trichloride (35.4 mg, 0.2314 mmol) at room temperature for 12 hour. The mixture was diluted with AcOEt and washed with saturated NaHCO₃ solution and brine, dried over Na₂SO₄ and evaporated in vacuo. The residue was suspended with AcOEt/heptane and the resulting precipitates were collected on the filter paper to give 1-(2,5-diphenyloxazol-4-yl)-5-methyl-4-(1H-1,2,4-triazol-1-yl)pyrimidin-2(1H)-one (37.0 mg, 80.6%) as a white solid. ¹H NMR (400 MHz, DMSO-d6) δ 9.48 (s, 1H), 8.59 (s, 1H), 8.47 (s, 1H), 8.17-8.16 (m, 2H), 7.67-7.64 (m, 5H), 7.53-7.47 (m, 3), 2.40 (s, 3H).

### 1-(2,5-diphenyl-1,3-oxazol-4-yl)-4-(2-hydroxyethoxy)-5-methyl-1,2-dihydropyrimidin-2-one. Compound 77

A mixture of 1-(2,5-diphenyloxazol-4-yl)-5-methyl-4-(1H-1,2,4-triazol-1-yl)pyrimidin-2(1H)-one (30 mg, 0.07568 mmol), 2,3,4,6,7,8,9,10-octahydropyrimido[1,2-a]azepine (0.02304 g, 0.1510 mmol) and ethane-1,2-diol (46.9 mg, 0.7555 mmol) in acetonitrile (1 mL) was stirred at 80° C. for 24 hour. The mixture was diluted with dichloromethane and washed with water and brine, dried over Na2SO4 and evaporated in vacuo. The residue was suspended into EtOAc and collected on a filter paper to give 1-(2,5-diphenyloxazol-4-yl)-4-(2-hydroxyethoxy)-5-methylpyrimidin-2(1H)-one (27.0 mg, 91.8%) as a white solid.

LCMS (ESI+): m/z 390.0, [M+H]⁺. ¹H NMR (400 MHz, CDCl₃) δ 8.12-8.10 (m, 2H), 7.62-7.60 (m, 2H), 7.53-7.50 (m, 3H), 7.46-7.41 (m, 4H), 4.69-4.67 (m, 2H), 4.06-4.02 (m, 2H), 2.05 (s, 3H).

### Synthesis of 2H,3H-furo[2,3-d]pyrimidin-2-one

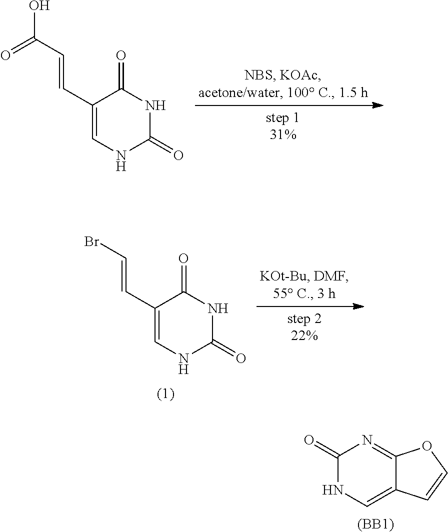

### 5-[(1E)-2-bromoethenyl]-1,2,3,4-tetrahydropyrimidine-2,4-dione

A solution of N-bromosuccinimide (0.513 g, 2.882 mmol, 1.05 eq) in a mixture of Acetone (10.5 ml, 21.0 vol) and water (10.5 ml, 21.0 vol) and added dropwise, over 90 min, into a boiling solution of (2E)-3-(2,4-dioxo-1,2,3,4-tetrahydropyrimidin-5-yl)prop-2-enoic acid (0.5 g, 2.745 mmol, 1.0 eq) and Potassium acetate (0.283 g, 2.884 mmol, 1.05 eq) in water (27.5 ml, 55.0 vol). The reaction mixture was then concentrated under reduced pressure to half of the initial volume and cooled to 4° C. for 16 h. The so-obtained precipitate was filtered off and washed with ice-water to give 5-[(1E)-2-bromoethenyl]-1,2,3,4-tetrahydropyrimidine-2,4-dione (0.194 g, 0.894 mmol, 31%).

1H NMR (300 MHz, DMSO-d6) δ 11.32 (s, 2H), 7.67 (s, 1H), 7.23 (d, J=13.5 Hz, 1H), 6.84 (d, J=13.5 Hz, 1H).

### 2H,3H-furo[2,3-d]pyrimidin-2-one

Under inert atmosphere, 5-[(1E)-2-bromoethenyl]-1,2,3,4-tetrahydropyrimidine-2,4-dione (0.204 g, 0.893 mmol, 0.999 eq) and Potassium tert-butoxide (1.304 g, 11.621 mmol, 13.0 eq) were suspended in Dimethylformamide anhydrous (19.4 ml, 100.0 vol) and the resulting mixture was stirred for 3 h at 55° C. The solid material was filtered off and the filtrate was neutralized with HCl in methanol. The volatiles were then removed under reduced pressure. The solid residue was washed with ice water and dried under reduced pressure to give 2H,3H-furo[2,3-d]pyrimidin-2-one (0.027 g, 0.198 mmol, 22%).

1H NMR (300 MHz, DMSO-d6) δ 12.14 (s, 1H), 8.34 (s, 1H), 7.70 (d, J=2.7 Hz, 1H), 6.74 (d, J=2.7 Hz, 1H).

### Synthesis of Compound 181.

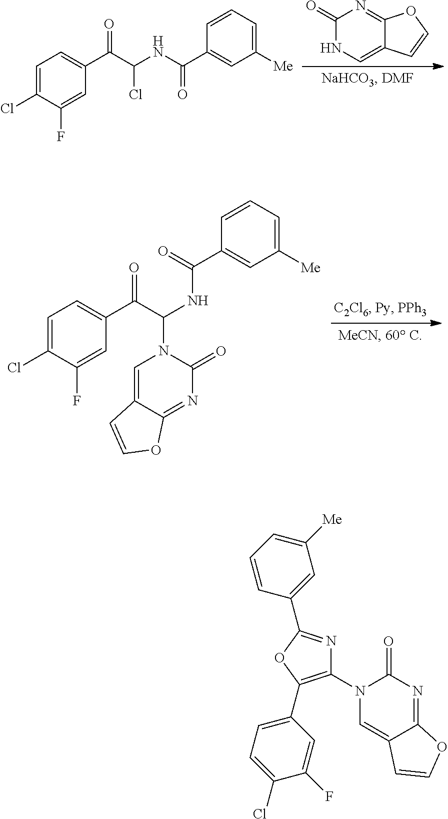 [SMILES: Cc1cccc(C(=O)NC(Cl)C(=O)c2ccc(Cl)c(F)c2)c1 ; O=c1nc2occc2c[nH]1 ; Cc1cccc(C(=O)NC(C(=O)c2ccc(Cl)c(F)c2)n2cc3ccoc3nc2=O)c1 ; Cc1cccc(-c2nc(-n3cc4ccoc4nc3=O)c(-c3ccc(Cl)c(F)c3)o2)c1]

### N-[2-(4-chloro-3-fluorophenyl)-2-oxo-1-{2-oxo-2H,3H-furo[2,3-d]pyrimidin-3-yl}ethyl]-3-methylbenzamide

N-[1-Chloro-2-(4-chloro-3-fluorophenyl)-2-oxoethyl]-3-methylbenzamide (0.029 g, 0.213 mmol, 0.906 eq) was added to a stirred solution of sodium bicarbonate (0.099 g, 1.178 mmol, 5.011 eq) and 2H,3H-furo[2,3-d]pyrimidin-2-one (0.1 g, 0.235 mmol, 1.0 eq) in Dimethylformamide (0.8 ml, 10.0 vol) at 0° C. The resulting mixture was stirred at 0° C. for 30 min and then for 18 h at RT. Full conversion, 80% of DP in RM according to UPLC analysis. Dichloromethane and water were added, layers were separated. The organic layer was washed with brine (3×), dried over sodium sulfate and concentrated under reduced pressure to give N-[2-(4-chloro-3-fluorophenyl)-2-oxo-1-{2-oxo-2H,3H-furo[2,3-d]pyrimidin-3-yl}ethyl]-3-methylbenzamide (0.095 g, 0.216 mmol, 85%)

LC-MS: [M+H]⁺=439.85.

3-[5-(4-chloro-3-fluorophenyl)-2-(3-methylphenyl)-1,3-oxazol-4-yl]-2H,3H-furo[2,3-d]pyrimidin-2-one. Compound 181. Under inert atmosphere, 4-chloro-N-(1-chloro-2-oxo-2-phenylethyl)benzamide (0.097 g, 0.205 mmol, 1.002 eq), Hexachloroethane (0.097 g, 0.41 mmol, 2.003 eq) and Triphenylphosphine (0.107 g, 0.408 mmol, 1.994 eq) were suspended in Acetonitrile anhydrous (1.8 ml, 20.0 vol). The resulting mixture was stirred for 10 minutes before adding pyridine (0.066 ml, 0.819 mmol, 4.004 eq). The resulting mixture was then stirred for 2 h at 60° C. Full conversion, 46% of desired in reaction mixture according to UPLC analysis. Dichloromethane and brine were added, layers were separated. The organic layer was washed with brine (3×), dried over sodium sulfate and concentrated under reduced pressure to give the crude product which was triturated with ethyl acetate to give (4) 3-[5-(4-chloro-3-fluorophenyl)-2-(3-methylphenyl)-1,3-oxazol-4-yl]-2H,3H-furo[2,3-d]pyrimidin-2-one (0.013 g, 0.031 mmol, 14%).

1H NMR (300 MHz, DMSO-d6) δ 8.81 (s, 1H), 8.03 (s, 1H), 7.98 (d, J=7.5 Hz, 1H), 7.88 (d, J=2.7 Hz, 1H), 7.80 (dd, J=10.4, 2.1 Hz, 1H), 7.69 (t, J=8.1 Hz, 1H), 7.48 (dt, J=14.0, 7.6 Hz, 2H), 7.35 (dd, J=8.6, 2.0 Hz, 1H), 6.89 (d, J=2.8 Hz, 1H), 2.44 (s, 3H).

LCMS: [M+H]⁺=422.06.

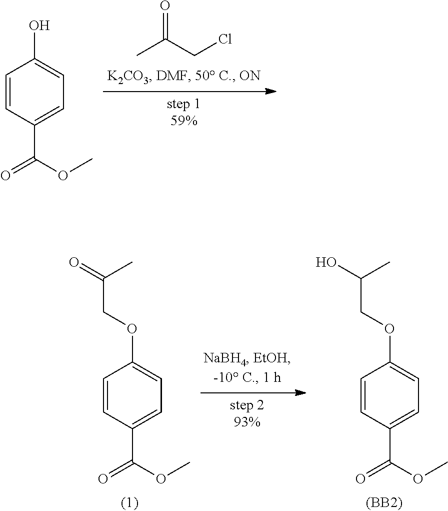

### methyl 4-(2-oxopropoxy)benzoate

A mixture of methyl 4-hydroxybenzoate (2.0 g, 13.145 mmol, 1.0 eq), chloroacetone (2.432 g, 26.270 mmol, 2.0 eq), potassium carbonate (5.450 g, 39.435 mmol, 3.0 eq) and dimethylformamide (40.0 ml, 20.0 eq) was stirred for 16 h at 50° C. The reaction mixture was cooled to room temperature, diluted with ethyl acetate, washed with saturated sodium bicarbonate solution and brine. Then organic layer was dried over sodium sulfate and concentrated under reduced pressure to give the crude product. The crude product was purified via FCC (cHx/AcOEt 1/0 up to 7/3 v/v) to give methyl 4-(2-oxopropoxy)benzoate (1.613 g, 13.15 mmol, 59%).

1H NMR (300 MHz, DMSO-d6) δ 7.90 (d, J=9.0 Hz, 2H), 7.02 (d, J=9.0 Hz, 2H), 4.95 (s, 2H), 3.82 (s, 3H), 2.17 (s, 3H).

### methyl 4-(2-hydroxypropoxy)benzoate

Methyl 4-(2-oxopropoxy)benzoate (1.613 g, 7.747 mmol, 1.0 eq) was dissolved in Ethanol (32.26 ml, 20.0 eq). The mixture was cooled to −10° C. and Sodium borohydride (0.352 g, 9.296 mmol, 1.2 eq) was added portionwise. The reaction mixture was stirred at −10° C. for 1 h. TLC (hex/AcOEt 8/2 v/v) showed full consumption of SM. 89% of DP in RM according to UPLC analysis. Water (20 ml) was added and the product was extracted with dichloromethane (3×). Combined organic layers were washed with brine, dried over sodium sulfate and concentrated under reduced pressure to give methyl 4-(2-hydroxypropoxy)benzoate (1.514 g, 7.75 mmol, 93%).

1H NMR (300 MHz, DMSO-d6) δ 7.91 (d, J=9.0 Hz, 2H), 7.05 (d, J=9.0 Hz, 2H), 4.93 (d, J=4.7 Hz, 1H), 4.02-3.93 (m, 1H), 3.92-3.85 (m, 2H), 3.82 (s, 3H), 1.16 (d, J=6.2 Hz, 3H).

### Synthesis of Compound 182.

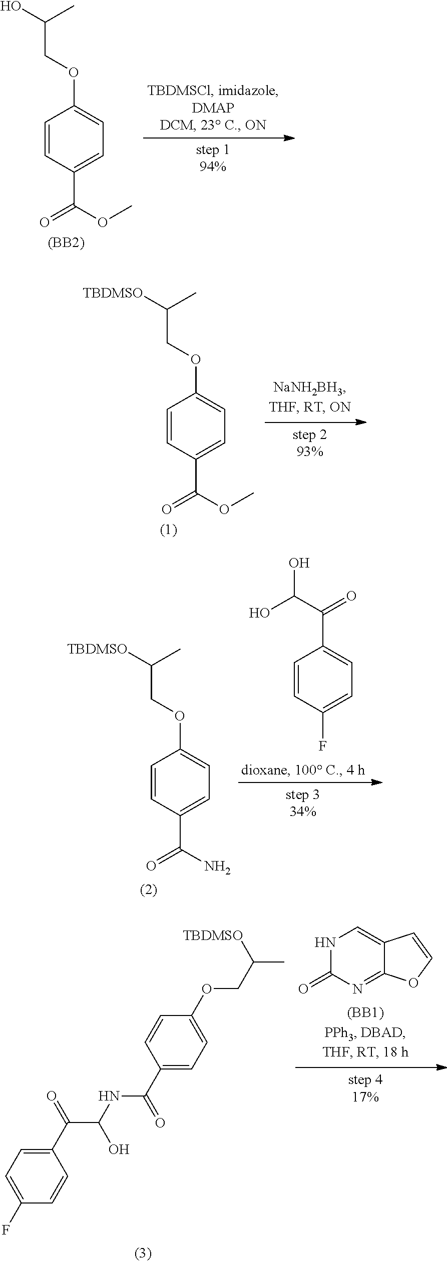

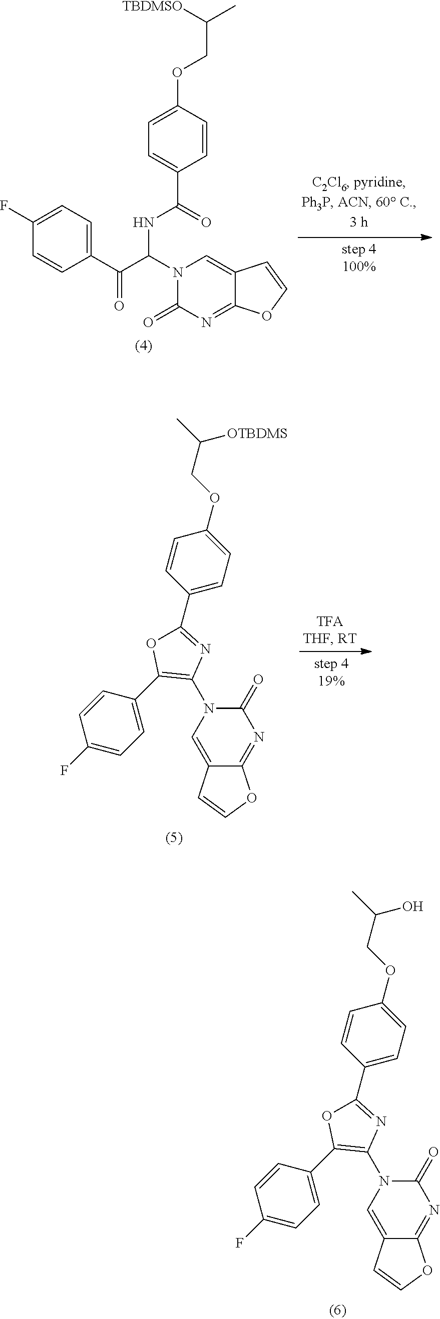 [SMILES: CC(COc1ccc(C(=O)NC(C(=O)c2ccc(F)cc2)n2cc3ccoc3nc2=O)cc1)O[Si](C)(C)C(C)(C)C ; CC(COc1ccc(-c2nc(-n3cc4ccoc4nc3=O)c(-c3ccc(F)cc3)o2)cc1)O[Si](C)(C)C(C)(C)C ; CC(O)COc1ccc(-c2nc(-n3cc4ccoc4nc3=O)c(-c3ccc(F)cc3)o2)cc1]

### methyl 4-{2-[(tert-butyldimethylsilyl)oxy]propoxy}benzoate

To a stirred mixture of methyl 4-(2-hydroxypropoxy)benzoate (1.514 g, 7.202 mmol, 1.0 eq) and imidazole (1.961 g, 28.807 mmol, 4.0 eq) in dry dichloromethane (22.71 ml, 15.0 vol) were added at RT tert-butyldimethylsilyl chloride (2.171 g, 14.403 mmol, 2.0 eq) together with DMAP (0.880 g, 7.202 mmol, 1.0 eq). The resulting mixture was stirred at RT for 16 h.

The reaction mixture was quenched by addition of water (25 ml). The product was extracted with dichloromethane (3×). The combined organic extracts were washed with water (2×) and brine, dried over sodium sulfate and concentrated under reduced pressure to give methyl 4-{2-[tert-butyldimethylsilyl)oxy]propoxy}benzoate (2.304 g, 7.20 mmol, 94%).

1H NMR (300 MHz, DMSO-d6) δ 7.91 (d, J=8.9 Hz, 2H), 7.03 (d, J=8.9 Hz, 2H), 4.23-4.11 (m, 1H), 4.01-3.84 (m, 2H), 3.82 (s, 3H), 1.18 (d, J=6.3 Hz, 3H), 0.86 (s, 9H), 0.07 (d, J=10.3 Hz, 6H).

### 4-{2-[(tert-butyldimethylsilyl)oxy]propoxy}benzamide

Sodium hydride (0.755 g, 18.884 mmol, 2.8 eq) was added to a solution of Borane-ammonia complex (0.451 g, 16.186 mmol, 2.4 eq) in Tetrahydrofuran anhydrous (46.07 ml, 20.0 vol) and the resulting mixture was stirred at RT for 30 min. Methyl 4-{2-[(tert-butyldimethylsilyl)oxy]propoxy}benzoate (1) (2.304 g, 6.744 mmol, 1.0 eq) was then added dropwise as a solution in THF (6 ml). The resulting mixture was then stirred at RT for 18 h. Water, brine and ethyl acetate were added to the reaction mixture, phases were separated. The aqueous layer was extracted with ethyl acetate (3×). Combined organic extracts were washed with brine, dried over sodium sulfate and concentrated under reduced pressure to give 4-{2-[(tert-butyldimethylsilyl)oxy]propoxy}benzamide (2.052 g, 6.74 mmol, 93%).

1H NMR (300 MHz, DMSO-d6) δ 7.90-7.79 (m, 3H), 7.18 (s, 1H), 6.96 (d, J=8.9 Hz, 2H), 4.21-4.11 (m, 1H), 3.97-3.82 (m, 2H), 1.19 (d, J=6.3 Hz, 3H), 0.87 (s, 9H), 0.09 (s, 3H), 0.06 (s, 3H).

### 4-{2-[(tert-butyldimethylsilyl)oxy]propoxy}-N-[2-(4-fluorophenyl)-1-hydroxy-2-oxoethyl]benzamide

4-Fluorophenylglyoxal hydrate (1.179 g, 6.929 mmol, 1.1 eq) and 4-{2-[(tert-butyldimethylsilyl)oxy]propoxy}benzamide (2.052 g, 6.30 mmol, 1.0 eq) were dissolved in Dioxane (30.78 ml, 15.0 vol) and the resulting mixture was stirred at 100° C. for 5 h. Dioxane was removed under reduced pressure to give the crude product which was purified via FCC (cHx/AcOEt 100/0 up to 7/3 v/v) to give 4-{2-[(tert-butyldimethylsilyl)oxy]propoxy}-N-[2-(4-fluorophenyl)-1-hydroxy-2-oxoethyl]benzamide (3) (1.047 g, 6.30 mmol, 34%).

1H NMR (300 MHz, DMSO-d6) δ 9.22 (d, J=7.9 Hz, 1H), 8.15-8.01 (m, 3H), 7.86 (d, J=8.9 Hz, 2H), 7.36 (t, J=8.9 Hz, 2H), 6.97 (d, J=8.9 Hz, 2H), 6.46 (d, J=8.1 Hz, 1H), 4.21-4.12 (m, 1H), 3.97-3.82 (m, 2H), 1.19-1.17 (m, 3H), 0.86 (s, 9H), 0.07 (d, J=10.1 Hz, 6H).

### 4-{2-[(tert-butyldimethylsilyl)oxy]propoxy}-N-[2-(4-fluorophenyl)-2-oxo-1-{2-oxo-2H,3H-furo[2,3-d]pyrimidin-3-yl}ethyl]benzamide

4-{2-[(tert-Butyldimethylsilyl)oxy]propoxy}-N-[2-(4-fluorophenyl)-1-hydroxy-2-oxoethyl]benzamide (3), 2H,3H-furo[2,3-d]pyrimidin-2-one (0.421 g, 1.949 mmol, 1.0 eq) and Triphenylphosphine (0.562 g, 2.144 mmol, 1.1 eq) were dried with toluene under vacuum for 1 h (3 times). The dried starting materials were then dissolved in Tetrahydrofuran anhydrous (9.47 ml, 10.0 vol). Di-tert-butyl azodicarboxylate (0.583 g, 2.534 mmol, 1.3 eq) was added in one portion and the resulting mixture was stirred for 18 h at RT. The solvent was removed under reduced pressure. The crude material was purified via FCC (DCM/AcOEt 100/0 up to 8/2 v/v) to give 4-{2-[(tert-butyldimethylsilyl)oxy]propoxy}-N-[2-(4-fluorophenyl)-2-oxo-1-{2-oxo-2H,3H-furo[2,3-d]pyrimidin-3-yl}ethyl]benzamide (0.194 g, 1.95 mmol, 17%).

1H NMR (300 MHz, DMSO-d6) δ 9.98 (d, J=8.4 Hz, 1H), 8.68 (s, 1H), 8.08-8.02 (m, 2H), 7.93 (dd, J=8.9, 1.2 Hz, 2H), 7.81 (d, J=2.7 Hz, 1H), 7.76 (d, J=8.1 Hz, 1H), 7.44 (t, J=8.8 Hz, 2H), 7.03 (d, J=8.9 Hz, 2H), 6.89 (dd, J=2.7, 0.6 Hz, 1H), 4.21-4.13 (m, 1H), 3.99-3.83 (m, 2H), 1.18 (d, J=6.3 Hz, 3H), 0.85 (s, 9H), 0.07 (d, J=10.8 Hz, 6H).

### 3-[2-(4-{2-[(tert-butyldimethylsilyl)oxy]propoxy}phenyl)-5-(4-fluorophenyl)-1,3-oxazol-4-yl]-2H,3H-furo[2,3-d]pyrimidin-2-one

4-{2-[(tert-Butyldimethylsilyl)oxy]propoxy}-N-[2-(4-fluorophenyl)-2-oxo-1-{2-oxo-2H,3H-furo[2,3-d]pyrimidin-3-yl}ethyl]benzamide (4) (0.194 g, 0.335 mmol, 1.0 eq) was suspended in Acetonitrile anhydrous (3.88 ml, 20.0 vol). Hexachloroethane (0.238 g, 1.004 mmol, 3.0 eq) and Triphenylphosphine (0.263 g, 1.004 mmol, 3.0 eq) were added. The resulting mixture was stirred for 10 minutes at room temperature and Pyridine (0.162 ml, 2.008 mmol, 6.0 eq) was added. Reaction was carried out at 60° C. for 2 h. The solvent was removed under reduced pressure. The crude 3-[2-(4-{2-[(tert-butyldimethylsilyl)oxy]propoxy}phenyl)-5-(4-fluorophenyl)-1,3-oxazol-4-yl]-2H,3H-furo[2,3-d]pyrimidin-2-one (0.709 g, 0.33 mmol, 98%) was used directly in the subsequent step without purification.

### 3-[5-(4-fluorophenyl)-2-[4-(2-hydroxypropoxy)phenyl]-1,3-oxazol-4-yl]-2H,3H-furo[2,3-d]pyrimidin-2-one. Synthesis of Compound 182

Trifluoroacetic acid (0.374 g, 3.282 mmol, 10.0 eq) was added to a 3-[2-(4-{2-[(tert-butyldimethylsilyl)oxy]propoxy}phenyl)-5-(4-fluorophenyl)-1,3-oxazol-4-yl]-2H,3H-furo[2,3-d]pyrimidin-2-one (0.709 g, 0.328 mmol, 1.0 eq) in Dichloromethane (21.27 ml, 30.0 vol). Resulting mixture was stirred, sealed for 18 h at RT. The solvent was removed under reduced pressure to give the crude product which was triturated with DMSO. The solid was filtered off and the filtrate was purified via prep-HPLC. The solid residue (70% purity via UPLC) was separately purified via FCC (DCM/MeOH I/O up to 95/5 v/v). Both fractions were combined after purification to give 3-[5-(4-fluorophenyl)-2-[4-(2-hydroxypropoxy)phenyl]-1,3-oxazol-4-yl]-2H,3H-furo[2,3-d]pyrimidin-2-one (0.028 g, 0.33 mmol, 19%) as a white solid.

1H NMR (400 MHz, DMSO-d6) δ 8.82 (s, 1H), 8.07 (d, J=8.9 Hz, 2H), 7.88 (d, J=2.6 Hz, 1H), 7.64-7.59 (m, 2H), 7.34 (t, J=8.9 Hz, 2H), 7.16 (d, J=8.9 Hz, 2H), 6.87 (d, J=2.7 Hz, 1H), 4.94 (d, J=4.7 Hz, 1H), 4.02-3.96 (m, 1H), 3.95-3.90 (m, 2H), 1.18 (d, J=6.2 Hz, 3H).

LCMS: [M+H]⁺=448.14

Additional Compounds Prepared in an Analogous Manner to Compound 182.

| | Exact mass | | |
|---|---|---|---|
| Compound | (g/mol) | Found | Assignment |
| 96 | 389.0567 | 390.0 | [M + H]+ |
| 183 | 355.0957 | 356.5 | [M + H]+ |
| 184 | 425.0379 | 426.08 | [M + H]+ |

### Synthesis of Compound 185.

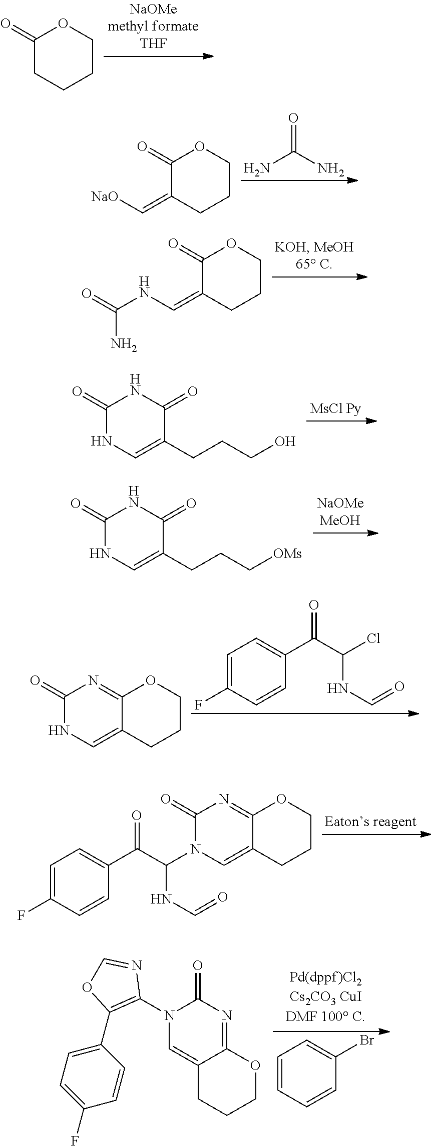

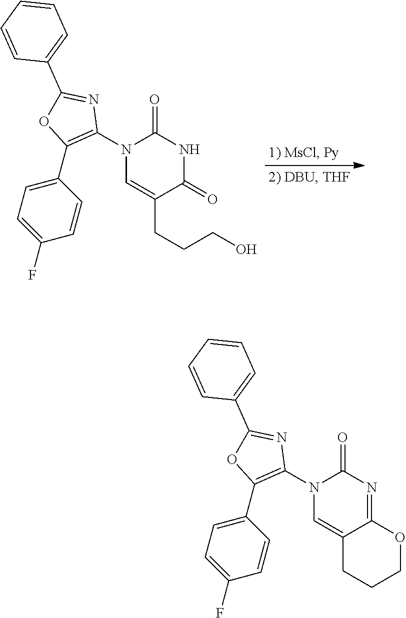 [SMILES: O=c1[nH]c(=O)n(-c2nc(-c3ccccc3)oc2-c2ccc(F)cc2)cc1CCCO ; O=c1nc2c(cn1-c1nc(-c3ccccc3)oc1-c1ccc(F)cc1)CCCO2]

### (Z)-1-((2-oxodihydro-2H-pyran-3 (4H)-ylidene)methyl)urea

To a stirred solution of tetrahydro-2H-pyran-2-one (5 g, 1 Eq, 0.05 mol) in THF (50 mL) at 0° C., was added NaOMe (4 g, 1.3 Eq, 0.06 mol) followed by Methyl formate (4 g, 4 mL, 1.3 Eq, 0.06 mol) dropwise. The reaction mixture was stirred at rt for 16 h. Reaction mixture was filtered, washed with THF and dried under vacuum to get sodium (Z)-(2-oxodihydro-2H-pyran-3(4H)-ylidene)methanolate (5 g, 0.03 mol, 70%) (highly higroscopic) as an Off white solid used for next step immediately.

To a stirred solution of urea (5 g, 2.5 Eq, 0.08 mol) in 3N HCl (40 mL) was added sodium (Z)-(2-oxodihydro-2H-pyran-3(4H)-ylidene)methanolate (5 g, 1 Eq, 0.03 mol) at 0° C., portion wise. The reaction mixture was stirred at rt for 16 h. The white solid formed was filtered and dried under vacuum. The solid compound was recrystallized from water (5 g crude dissolved in water (140 mL) reflux for 1 hr to get clear solution, then cooled to rt, white crystals were formed and filtered, dried under vacuum to get (Z)-1-((2-oxodihydro-2H-pyran-3 (4H)-ylidene)methyl)urea (2.5 g, 15 mmol, 40%) as a white solid.

LCMS: [M−H]⁻=m/z 169.4.

### 5-(3-hydroxypropyl)pyrimidine-2,4(1H,3H)-dione

To a stirred solution of KOH (1.2 g, 1.5 Eq, 22 mmol) in Methanol (30 mL) at rt, was added (Z)-1-((2-oxodihydro-2H-pyran-3(4H)-ylidene)methyl)urea (2.5 g, 1 Eq, 15 mmol) at one lot. The reaction mixture was stirred at 65° C., for 4 hrs. The reaction mixture was filtered and washed with methanol. The solid compound was dissolved in water (10 mL) acidified with 3N HCl (ph˜3), solid ppt was formed, filtered and washed with water dried under vacuum to get compound. The compound was recrystallized from water (white solid was diluted in water (20 ml) and heated to reflux for 1 hr to get clear solution. Then the solution was allowed to cool to rt and kept at rt for 16 h. white crystals formed were filtered and dried under vacuum to get pure 5-(3-hydroxypropyl)pyrimidine-2,4(1H,3H)-dione (1.1 g, 6.5 mmol, 44%) as a white solid.

1H NMR (400 MHz, DMSO-d6) δ ppm 10.97 (s, 1H), 10.60 (d, J=4.4 Hz, 1H), 7.17 (d, J=5.6 Hz, 1H), 4.60-4.15 (m, 1H), 3.36 (t, J=6.4 Hz, 2H), 2.18 (t, J=7.2 Hz, 2H), 1.57-1.50 (m, 2H).

### 3,5,6,7-tetrahydro-2Hpyrano[2,3-d]pyrimidin-2-one

To a stirred solution of 5-(3-hydroxypropyl)pyrimidine-2,4(1H,3H)-dione (2 g, 1 Eq, 0.01 mol) in Pyridine (40 mL) at −10° C., was added MsCl (2.01 g, 1.36 mL, 1 Eq, 17.6 mmol) drop wise. The reaction mixture was stirred at 0° C., for 2 hr. The reaction mixture was monitored by TLC (5% MeOH/DCM SM rf: 0.2, Product rf: 0.4), SM Completed non polar spot was observed. Work up: The reaction mixture was poured in to ice water (200 mL) and stirred for 10 mins, the white solid ppt formed was filtered and dried under vacuum to get 3-(2,4-dioxo-1,2,3,4-tetrahydropyrimidin-5-yl)propyl methanesulfonate (2.2 g, 8.9 mmol, 80%) as a white solid.

To a stirred solution of 3-(2,4-dioxo-1,2,3,4-tetrahydropyrimidin-5-yl)propyl methanesulfonate (2.2 g, 1 Eq, 8.9 mmol) in Methanol (50 mL) at 0° C., was added Sodium methoxide (0.48 g, 1 Eq, 8.9 mmol). The reaction mixture was heated to 65° C. for 4 hr. The reaction mixture was monitored by TLC, SM Completed polar spot was observed. Work up: The reaction mixture was diluted with DCM (50 mL) and filtered. Filtrate was concentrated to get crude compound Purification: Crude compound was triturated with IPA (10 mL), filtered and dried under vacuum to get 3,5,6,7-tetrahydro-2Hpyrano[2,3-d]pyrimidin-2-one (1.2 g, 7.9 mmol, 89%) as an Off white solid.

### N-(2-(4-fluorophenyl)-2-oxo-1-(2-oxo-6,7-dihydro-2H-pyrano[2,3-d]pyrimidin-3(5H)-yl)ethyl)formamide

To a stirred solution of 3,5,6,7-tetrahydro-2H-pyrano[2,3-d]pyrimidin-2-one (1.9 g, 1.1 Eq, 12 mmol) in DMF (19 mL) at 0° C., was added TEA (3.4 g, 4.4 mL, 3 Eq, 33 mmol), followed by N-(1-chloro-2-(4-fluorophenyl)-2-oxoethyl)formamide (2.4 g, 1 Eq, 11 mmol) diluted with DMF (24 mL) drop wise. The reaction mixture was stirred at rt for 16 h. The reaction mixture was monitored by TLC, sm completed product was observed. Work up: The reaction mixture was concentrated to get crude, triturated with ethyl acetate and filtered, dried under vacuum to get solid, and it was dissolved in water and stirred for 30 mins, filtered and dried under vacuum to get N-(2-(4-fluorophenyl)-2-oxo-1-(2-oxo-6,7-dihydro-2H-pyrano[2,3-d]pyrimidin-3(5H)-yl)ethyl)formamide (2 g, 6 mmol, 50%, 95% Purity) as a brown solid.

LCMS: [M−H]⁻=330.1 m/z

### 3-(5-(4-fluorophenyl)oxazol-4-yl)-3,5,6,7-tetrahydro-2H-pyrano[2,3-d]pyrimidin-2-one

To a stirred solution of N-(2-(4-fluorophenyl)-2-oxo-1-(2-oxo-6,7-dihydro-2H-pyrano[2,3-d]pyrimidin-3(5H)-yl)ethyl)formamide (3.5 g, 1 Eq, 11 mmol) in 100 mL single neck RBF, was added Eaton's reagent (21 mL). The reaction mixture was heated at 70° C., for 2 h. The reaction mixture was monitored by TLC, sm completed product was observed. Work up: The reaction mixture was poured into crushed ice and basified with NaHCO₃. Aq layer was extracted with 10% MeOH in DCM. Total organic layers were concentrated to get crude compound. Purification: Crude compound was purified by flash chromatography, and the product eluted in 2%-4% MeOH in DCM, Pure fractions were concentrated to get pure 3-(5-(4-fluorophenyl)oxazol-4-yl)-3,5,6,7-tetrahydro-2H-pyrano[2,3-d]pyrimidin-2-one (1.6 g, 5.1 mmol, 48%) as an Off white solid.

LCMS: [M+H]⁺=m/z 314.1

### 1-(5-(4-fluorophenyl)-2-phenyloxazol-4-yl)-5-(3-hydroxypropyl)pyrimidine-2,4(1H,3H)-dione

To a stirred solution of 3-(5-(4-fluorophenyl)oxazol-4-yl)-3,5,6,7-tetrahydro-2H-pyrano[2,3-d]pyrimidin-2-one (200 mg, 1 Eq, 638 μmol) in DMF (4 mL), was added Cs₂CO₃ (624 mg, 3 Eq, 1.92 mmol), followed by bromobenzene (200 mg, 134 μL, 2 Eq, 1.28 mmol) and purged with argon for 15 min, then was added CuI (12.2 mg, 0.1 Eq, 63.8 μmol) and Pd(dppf)Cl₂ (46.7 mg, 0.1 Eq, 63.8 μmol) and purged for 5 min. The reaction mixture was heated at 100° C. for 3 h. The reaction mixture was diluted with water, solid was formed and filtered dried under vacuum to get crude solid compound. Crude compound was purified by flash chromatography by eluting with 2%-5% MeOH/Ethyl acetate to get 1-(5-(4-fluorophenyl)-2-phenyloxazol-4-yl)-5-(3-hydroxypropyl)pyrimidine-2,4(1H,3H)-dione (140 mg, 328 μmol, 51.3%, 95.34% Purity). an Off white solid.

LCMS: (+esi)[M+H]=408.1

¹H NMR: 1H NMR (400 MHz, DMSO-d6) δ=11.73 (s, 1H), 8.13 (dd, J=2.9, 6.6 Hz, 2H), 7.75-7.68 (m, 2H), 7.66 (s, 1H), 7.64-7.59 (m, 3H), 7.41 (t, J=8.9 Hz, 2H), 4.43 (t, J=5.3 Hz, 1H), 3.44-3.37 (m, 2H), 2.29 (br t, J=7.5 Hz, 2H), 1.66-1.57 (m, 2H).

### 3-(1-(5-(4-fluorophenyl)-2-phenyloxazol-4-yl)-2,4-dioxo-1,2,3,4-tetrahydropyrimidin-5-yl)propyl methanesulfonate

To a stirred solution of 1-(5-(4-fluorophenyl)-2-phenyloxazol-4-yl)-5-(3-hydroxypropyl)pyrimidine-2,4(1H,3H)-dione (130 mg, 1 Eq, 319 μmol) in Pyridine (2.5 mL), was added MsCl (54.8 mg, 37.0 μL, 1.5 Eq, 479 μmol) at 0° C. The resulting reaction mixture was allowed to stir for 2 h. The reaction mixture was then poured in crushed ice and the solid separated was filtered and washed with cold water and MTBE. The solid was then re dissolved in 10% methanol in chloroform, dried over anhydrous sodium sulfate, filtered and concentrated under reduced pressure to get 3-(1-(5-(4-fluorophenyl)-2-phenyloxazol-4-yl)-2,4-dioxo-1,2,3,4-tetrahydropyrimidin-5-yl)propyl methanesulfonate (130 mg, 0.24 mmol, 76%, 90% Purity) an Off white solid.

LCMS: (+esi) [M+H]+: 486.1

1H NMR (400 MHz, DMSO-d) δ=11.79-11.76 (m, 1H), 8.13 (dd, J=3.0, 6.7 Hz, 2H), 7.76-7.71 (m, 3H), 7.65-7.58 (m, 3H), 7.40 (t, J=8.9 Hz, 2H), 4.22 (t, J=6.2 Hz, 2H), 3.15 (s, 3H), 2.37 (br t, J=7.5 Hz, 2H), 1.94-1.86 (m, 2H).

### 3-(5-(4-fluorophenyl)-2-phenyloxazol-4-yl)-3,5,6,7-tetrahydro-2H-pyrano[2,3-d]pyrimidin-2-one. Compound 185

To a stirred solution of 3-(1-(5-(4-fluorophenyl)-2-phenyloxazol-4-yl)-2,4-dioxo-1,2,3,4-tetrahydropyrimidin-5-yl)propyl methanesulfonate (130 mg, 1 Eq, 268 μmol) in THF (3.6 mL) was added a solution of DBU (61.6 mg, 405 μmol) in THF (1 mL) at rt, and the resulting reaction mixture was stirred at 65° C. for 30 min. The reaction mixture was cooled to room temperature and diluted with 10% methanol in dichloromethane and washed with cold water, dried over anhydrous sodium sulfate, filtered and evaporated under reduced pressure to yield 90 mg of crude compound, which was purified using flash chromatography by eluting with (3%-5%) methanol in ethyl acetate to 3-(5-(4-fluorophenyl)-2-phenyloxazol-4-yl)-3,5,6,7-tetrahydro-2H-pyrano[2,3-d]pyrimidin-2-one (25 mg, 62 μmol, 23%, 96.34% Purity) as an off white solid.

LCMS: (+esi) [M+H]+=390.0

¹H NMR (400 MHz, DMSO-d6) δ=8.23-7.98 (m, 3H), 7.80-7.44 (m, 5H), 7.43-7.32 (m, 2H), 4.46 (br t, J=4.8 Hz, 2H), 2.60 (br s, 2H), 1.95 (br s, 2H).

### Synthesis of Compound 92.

3-(5-(4-fluorophenyl)-2-phenyloxazol-4-yl)-7-methyl-3,5,6,7-tetrahydro-2H-pyrano[2,3-d]pyrimidin-2-one was prepared in a similar fashion to compound 75, starting with 6-methyltetrahydro-2H-pyran-2-one. LCMS: (+esi) [M+H]+=404.1

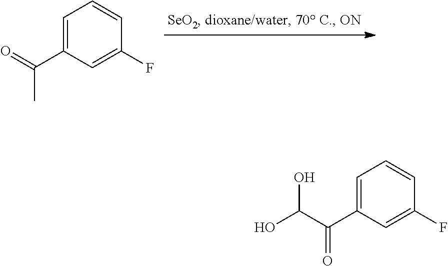

### 1-(3-fluorophenyl)-2,2-dihydroxyethan-1-one

3′-Fluoroacetophenone (2.0 g, 14.5 mmol, 1.0 eq) was dissolved in dioxane (12.0 mL) and water (2.0 mL). Selenium dioxide (3.21 g, 29.0 mmol, 2.0 eq) was added, and the reaction was stirred for 16 hours at 70° C. Upon cooling to room temperature, the reaction mixture was filtered through a pad of celite and the filtrate was concentrated. Water (50 mL) was added to the resulting oil, and the mixture was heated at reflux for 24 hours. Upon cooling to 0° C., a precipitate formed, and it was collected by vacuum filtration. The solid was washed with cold water and dried under vacuum to give 1-(3-fluorophenyl)-2,2-dihydroxyethan-1-one (1.65 g, 9.70 mmol, 67%) as a solid. This material was used directly in the next step.

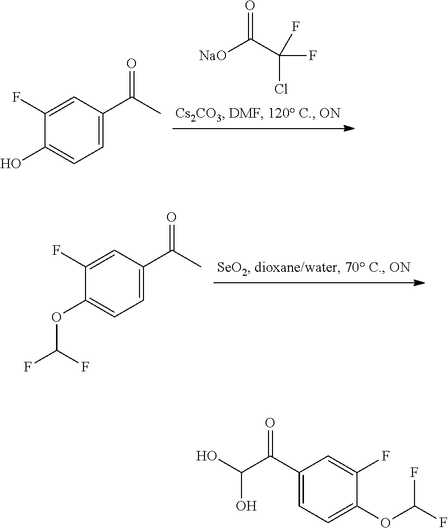

### 1-[4-(difluoromethoxy)-3-fluorophenyl]ethan-1-one

Under an inert atmosphere of argon, 3′-fluoro-4′-hydroxyacetophenone (5.0 g, 32.4 mmol, 1.0 eq), sodium chlorodifluoroacetate (7.42 g, 48.7 mmol, 1.5 eq), and cesium carbonate (21.1 g, 64.9 mmol, 2.0 eq) were suspended in DMF (50.0 mL). The reaction vessel was equipped with a carbon dioxide outlet. Then, the reaction was stirred for 16 hours at 120° C. Upon cooling to room temperature, the resulting mixture was diluted with ethyl acetate (100 mL) and washed with brine (3×100 mL). The organic layers were combined, dried over sodium sulfate, and concentrated under reduced pressure. The obtained crude material was purified by flash column silica gel chromatography (DCM, 100%) to give 1-[4-(difluoromethoxy)-3-fluorophenyl]ethan-1-one (2.9 g, 14.2 mmol, 44%).

¹H NMR (300 MHz, DMSO-d6) δ ppm 7.94 (dd, J=11.4, 2.1 Hz, 1H), 7.87 (ddd, J=8.5, 2.1, 1.1 Hz, 1H), 7.60-7.46 (m, 1H), 7.41 (t, J=72.7 Hz, 1H), 2.59 (s, 3H).

### 1-(4-chloro-3-fluorophenyl)-2,2-dihydroxyethan-1-one

1-[4-(Difluoromethoxy)-3-fluorophenyl]ethan-1-one (2.9 g, 14.2 mmol, 1.0 eq) was dissolved in dioxane (17.4 mL) and water (2.9 mL). Selenium dioxide (3.15 g, 28.4 mmol, 2.0 eq) was added, and the reaction was stirred for 16 hours at 70° C. The reaction mixture was filtered through a pad of celite, and the filtrate was concentrated. Water (15 mL) was added to the resulting oil, and the mixture was heated to reflux for 24 hours. Upon cooling to 0° C., a precipitate formed. This material was collected by vacuum filtration, washed with cold water, and dried under vacuum to give 1-(4-chloro-3-fluorophenyl)-2,2-dihydroxyethan-1-one (1.2 g, 5.87 mmol, 36%) as a beige solid. This material was used directly in the next reaction.

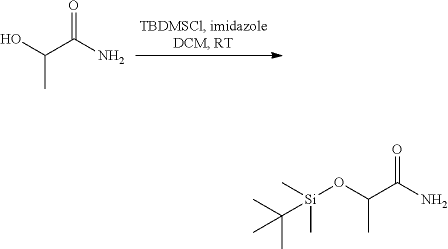

### 2-[(tert-butyldimethylsilyl)oxy]propanamide

To a stirred mixture of lactamide (2.0 g, 22.4 mmol, 1.0 eq) and imidazole (1.99 g, 29.2 mmol, 1.3 eq) in dry dichloromethane (30.0 mL) was added tert-butyldimethylsilyl chloride (4.40 g, 29.2 mmol, 1.3 eq). The resulting mixture was stirred at room temperature for 16 hours. Water (25 ml) was added to the reaction mixture. The mixture was extracted with DCM (3×25 mL). The combined organic extracts were washed with water (2×20 mL) and brine (1×50 mL), dried over anhydrous sodium sulphate, and concentrated under reduced pressure. The crude material was purified by silica gel column chromatography (0-5% MeOH in DCM) to afford 2-[(tert-butyldimethylsilyl)oxy]propanamide (4.3 g, 21.1 mmol, 94%).

¹H NMR (300 MHz, Chloroform-d) δ ppm 6.60 (s, 1H), 5.64 (d, J=25.1 Hz, 1H), 4.21 (q, J=6.8 Hz, 1H), 1.39 (d, J=6.8 Hz, 3H), 0.92 (s, 9H), 0.11 (d, J=2.7 Hz, 6H).

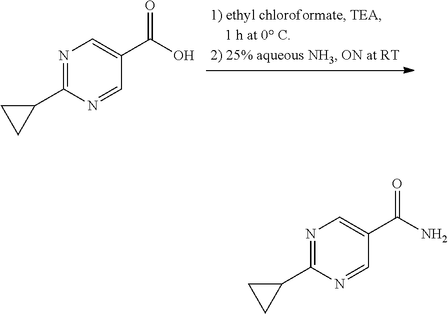 [SMILES: O=C(O)c1cnc(C2CC2)nc1 ; NC(=O)c1cnc(C2CC2)nc1]

### 2-cyclopropylpyrimidine-5-carboxamide

2-Cyclopropylpyrimidine-5-carboxylic acid (2.45 g, 14.9 mmol, 1.0 eq) was dissolved in anhydrous THF (36.8 mL). Triethylamine (2.50 mL, 17.9 mmol, 1.2 eq) was added and the resulting mixture was cooled to 0° C. Ethyl chloroformate (1.78 g, 16.4 mmol, 1.1 eq) was added dropwise, and the reaction mixture was stirred at 0° C. for 2 hours. Then, 25% aqueous ammonia (2.93 mL, 74.6 mmol, 5.0 eq) was added, and the mixture was allowed to warm to room temperature and stirred for 16 hours. The reaction mixture was diluted with ethyl acetate and the layers were separated. The organic layer was washed with saturated aqueous sodium bicarbonate and brine, dried over anhydrous sodium sulphate, and concentrated under reduced pressure to give 2-cyclopropylpyrimidine-5-carboxamide (0.8 g, 4.81 mmol, 32%) as a light-yellow solid.

¹H NMR (300 MHz, DMSO-d6) δ ppm 9.00 (s, 2H), 7.92 (d, J=151.5 Hz, 2H), 2.32-2.22 (m, 1H), 1.19-1.00 (m, 4H).

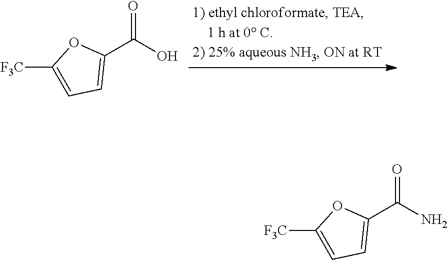 [SMILES: O=C(O)c1ccc(C(F)(F)F)o1 ; NC(=O)c1ccc(C(F)(F)F)o1]

### 5-(trifluoromethyl)furan-2-carboxamide

5-(Trifluoromethyl)-2-furoic acid (1.0 g, 5.55 mmol, 1.0 eq) was dissolved in THF (15.0 mL). Triethylamine (0.937 ml, 6.66 mmol, 1.2 eq) was added. The mixture was cooled to 0° C., and ethyl chloroformate (0.436 mL, 6.66 mmol, 1.2 eq) was added slowly. At this time, a 25% solution of ammonia in water (4.75 mL, 27.8 mmol, 5.0 eq) was added, and the resulting mixture was stirred for 16 hours at room temperature. Ethyl acetate was added to the reaction mixture and the layers were separated. The organic phase was washed with sodium bicarbonate and brine, dried over sodium sulfate, and concentrated under reduced pressure to give 5-(trifluoromethyl)furan-2-carboxamide (0.855 g, 4.77 mmol, 77%) as a colorless solid.

¹H NMR (300 MHz, DMSO-d6) δ ppm 8.12 (s, 1H), 7.73 (s, 1H), 7.36 (dt, J=3.6, 1.2 Hz, 1H), 7.29 (dd, J=3.7, 1.0 Hz, 1H).

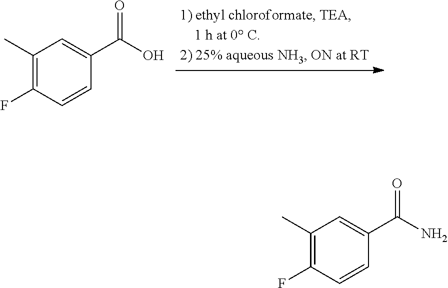

4-Fluoro-3-methylbenzoic acid (2.5 g, 16.2 mmol, 1.0 eq) was dissolved in THF (37.5 mL). Triethylamine (2.71 mL, 19.5 mmol, 1.2 eq) was added, and the resulting mixture was cooled to 0° C. Ethyl chloroformate (1.27 mL, 19.5 mmol, 1.2 eq) was added dropwise, and the reaction mixture was stirred at 0° C. for 1 hour. A 25% aqueous ammonia solution (40 mL, 40.5 mmol, 5.0 eq) was added and the mixture was allowed to warm to room temperature and stirred for 1 hour. Ethyl acetate was added to the reaction mixture and the layers were separated. The organic phase was washed with sodium bicarbonate and brine, dried over sodium sulfate, and concentrated under reduced pressure to give 4-fluoro-3-methylbenzamide (1.71 g, 11.2 mmol, 55%) as a white solid.

¹H NMR (300 MHz, DMSO-d6) δ ppm 7.95 (s, 1H), 7.83 (dd, J=7.8, 2.3 Hz, 1H), 7.74 (ddd, J=7.9, 5.1, 2.4 Hz, 1H), 7.36 (s, 1H), 7.21 (dd, J=9.6, 8.5 Hz, 1H), 2.27 (d, J=2.0 Hz, 3H).

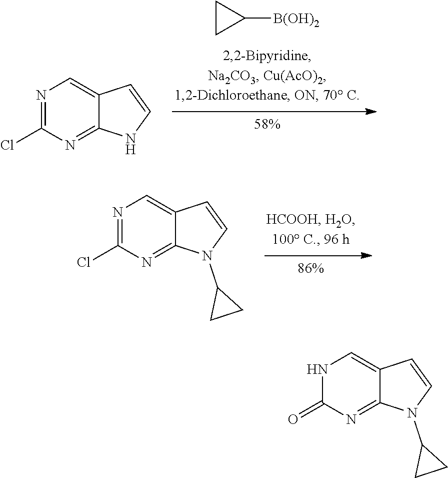 [SMILES: OB(O)C1CC1 ; Clc1ncc2cc[nH]c2n1 ; Clc1ncc2ccn(C3CC3)c2n1 ; O=c1nc2c(ccn2C2CC2)c[nH]1]

### 2-chloro-7-cyclopropyl-7H-pyrrolo[2,3-d]pyrimidine

2-Chloro-7H-pyrrolo[2,3-d]pyrimidine (10.0 g, 65.1 mmol, 1.0 eq), cyclopropylboronic acid (5.59 g, 65.1 mmol, 1.0 eq), copper(II) acetate (11.8 g, 65.1 mmol, 1.0 eq), 2,2-bipyridine (10.2 g, 65.1 mmol, 1.0 eq), sodium carbonate (13.9 g, 130 mmol, 2.0 eq), and molecular sieves (4 Å, powder) were combined in a 3-necked flask. The reaction vessel was filled with argon before anhydrous dichloroethane (200 mL) was added. Next, the reaction mixture was bubbled with 02 for 30 minutes while stirring at room temperature followed by stirring for 2 hours at 70° C. (mixture was bubbled with 02 for the whole time). After this time, the reaction was left for 16 hours at the same temperature under 02 atmosphere. The mixture was cooled to room temperature and quenched with 1 M HCl (aqueous, 400 mL). The water layer was extracted with dichloromethane. The organic extracts were combined, dried over anhydrous Na₂SO₄, filtered, and concentrated under reduced pressure to obtain a crude material as dark green solid. This material was then purified by flash silica gel column chromatography (0-5% MeOH in DCM) to give 2-chloro-7-cyclopropyl-7H-pyrrolo[2,3-d]pyrimidine (7.3 g, 37.7 mmol, 58%).

¹H NMR (300 MHz, DMSO-d6) δ ppm 8.90 (s, 1H), 7.61 (d, J=3.7 Hz, 1H), 6.64 (d, J=3.7 Hz, 1H), 3.71-3.49 (m, 1H), 1.18-0.94 (m, 4H).

### 7-cyclopropyl-2H,3H,7H-pyrrolo[2,3-d]pyrimidin-2-one

2-Chloro-7-cyclopropyl-7H-pyrrolo[2,3-d]pyrimidine (7.3 g, 37.7 mmol, 1.0 eq) was dissolved in a mixture of water (0.747 mL, 41.5 mmol, 1.1 eq) and formic acid (28.4 mL, 754 mmol, 20.0 eq). The reaction mixture was stirred for 4 days at 100° C. After each day of heating, additional portions of formic acid and water were added (1.1 eq water and 20.0 eq of formic acid). After cooling to room temperature, EtOAc was added. A precipitate was not observed so the reaction was cooled to 0° C. After stirring for 5 minutes, a precipitate was observed. The formed solid was collected by vacuum filtration and washed with EtOAc to give 7-cyclopropyl-2H,3H,7H-pyrrolo[2,3-d]pyrimidin-2-one (5.8 g, 32.4 mmol, 86%) as a light brown solid.

¹H NMR (300 MHz, DMSO-d6) δ ppm 8.71 (s, 1H), 7.32 (d, J=3.8 Hz, 1H), 6.55 (d, J=3.8 Hz, 1H), 3.41 (dq, J=7.3, 4.1, 3.6 Hz, 1H), 1.03 (dd, J=4.6, 1.7 Hz, 4H).

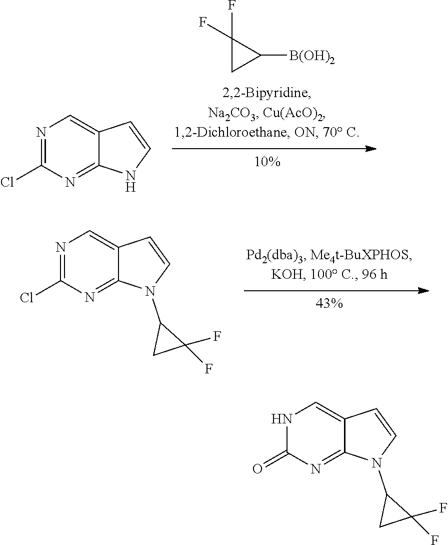 [SMILES: OB(O)C1CC1(F)F ; Clc1ncc2cc[nH]c2n1 ; FC1(F)CC1n1ccc2cnc(Cl)nc21 ; O=c1nc2c(ccn2C2CC2(F)F)c[nH]1]

### 2-chloro-7-(2,2-difluorocyclopropyl)-7H-pyrrolo[2,3-d]pyrimidine

2-Chloro-7H-pyrrolo[2,3-d]pyrimidine (0.40 g, 2.61 mmol, 1.0 eq), 2,2-difluoro-cyclopropaneboronic acid (0.317 g, 2.61 mmol, 1.0 eq), copper(II) acetate (0.757 g, 4.17 mmol, 1.6 eq), 2,2′-dipyridyl (0.61 g, 3.91 mmol, 1.5 eq), sodium carbonate (0.828 g, 7.81 mmol, 3.0 eq), and 4 Å molecular sieves (150 mg, powdered, activated before use) were placed in 3-necked flask charged with a reflux condenser. The reaction vessel was evacuated and backfilled with argon before anhydrous dichloroethane (16.0 mL) was added. Next, the reaction mixture was bubbled with 02 for 30 min and then heated to 70° C. and kept stirring with 02 bubbling for 3 hours. After this time, the reaction was left for 16 hours at 70° C. under 02 atmosphere. The reaction mixture was cooled down to room temperature, quenched with saturated aqueous NH₄Cl, and extracted twice with dichloromethane. The combined organic layers were dried over Na₂S0₄ and evaporated to obtain the crude material as a dark green solid. The crude material was purified by silica gel column chromatography (0-5% MeOH in DCM) to give 2-chloro-7-(2,2-difluorocyclopropyl)-7H-pyrrolo[2,3-d]pyrimidine (0.059 g, 0.252 mmol, 10%).

¹H NMR (300 MHz, DMSO-d6) δ ppm 8.98 (s, 1H), 8.11-7.34 (m, 1H), 6.75 (d, J=3.7 Hz, 1H), 4.52-4.29 (m, 1H), 2.48-2.35 (m, 2H).

### 7-(2,2-difluorocyclopropyl)-2H,3H,7H-pyrrolo[2,3-cl]pyrimidin-2-one

A mixture of 2-chloro-7-(2,2-difluorocyclopropyl)-7H-pyrrolo[2,3-d]pyrimidine (0.030 g, 0.128 mmol, 1.0 eq), 3.5 M KOH (in water, 0.220 mL, 5.0 eq), Me₄tButylXphos (0.0062 g, 0.0128 mmol, 0.1 eq), and tris(dibenzylideneacetone)dipalladium(0) (0.006 g, 0.006 mmol, 0.05 eq) in anhydrous dioxane (0.75 mL) was degassed with argon. The resulting mixture was stirred at 100° C. for 2.5 hours. Formic acid (0.003 mL, 10.0 eq) followed by water (1 mL) were added to the mixture then it was stirred for 5 minutes. The mixture was then subjected to the reverse phase column chromatography (C18-SiO₂, 0-30% MeCN in water) to provide 7-(2,2-difluorocyclopropyl)-2H,3H,7H-pyrrolo[2,3-d]pyrimidin-2-one (0.012 g, 0.056 mmol, 44%) as a white solid.

¹H NMR (300 MHz, DMSO-d6) δ ppm 11.73 (s, 1H), 8.23 (s, 1H), 7.15 (d, J=4.0 Hz, 1H), 6.30 (d, J=4.0 Hz, 1H), 4.05 (dt, J=10.1, 8.0 Hz, 1H), 2.41-2.12 (m, 2H).

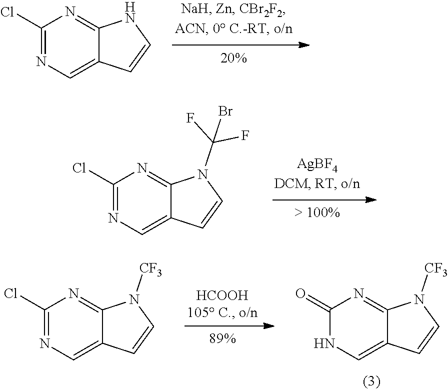

### 7-(bromodifluoromethyl)-2-chloro-7H-pyrrolo[2,3-d]pyrimidine

2-Chloro-7H-pyrrolo[2,3-d]pyrimidine (25.0 g, 163 mmol, 1.0 eq) was suspended in anhydrous MeCN (500 mL). The resulting suspension was cooled to 0° C., and sodium hydride (9.77 g, 244 mmol, 1.5 eq) was added in portions. The reaction mixture was then stirred for 1 h at 0° C. Zinc (0.16 g, 2.44 mmol, 0.015 eq) was then added and 10 minutes later dibromodifluoromethane (16.4 mL, 195 mmol, 1.2 eq) was added. The mixture was allowed to warm to room temperature and stirred for 16 hours. The solvent was removed under reduced pressure, and the residue was suspended in DCM. The formed solid precipitate was filtered off and washed with DCM. The filtrate was concentrated under reduced pressure to give the crude product. The crude product was then purified by silica gel column chromatography (100% DCM) to give 7-(bromodifluoromethyl)-2-chloro-7H-pyrrolo[2,3-d]pyrimidine (9.5 g, 33.0 mmol, 20%) as a white solid.

¹H NMR (400 MHz, DMSO-d6) δ ppm 9.13 (s, 1H), 8.00 (d, J=4.0 Hz, 1H), 7.03 (d, J=4.0 Hz, 1H).

### 2-chloro-7-(trifluoromethyl)-7H-pyrrolo[2,3-d]pyrimidine

7-(Bromodifluoromethyl)-2-chloro-7H-pyrrolo[2,3-d]pyrimidine (9.5 g, 33.0 mmol, 1.0 eq) was dissolved in dichloromethane (143 mL) and silver tetrafluoroborate (7.70 g, 39.6 mmol, 1.2 eq) was added. The resulting mixture was stirred at room temperature for 16 hours. The reaction mixture was diluted with DCM and filtered through a pad of celite eluting with DCM. The filtrate was concentrated under reduced pressure to give 2-chloro-7-(trifluoromethyl)-7H-pyrrolo[2,3-d]pyrimidine (8.86 g, 38.8 mmol, 118%) as a greyish solid.

¹H NMR (400 MHz, DMSO-d6) δ ppm 9.15 (s, 1H), 8.04 (d, J=4.0 Hz, 1H), 7.01 (d, J=4.0 Hz, 1H).

### 7-(trifluoromethyl)-2H,3H,7H-pyrrolo[2,3-d]pyrimidin-2-one

2-Chloro-7-(trifluoromethyl)-7H-pyrrolo[2,3-d]pyrimidine (8.86 g, 38.8 mmol, 1.0 eq) was dissolved in formic acid (146 mL, 3.88 mol, 100 eq). Water (34.9 mL, 1.94 mol, 50.0 eq) was added, and the reaction mixture was stirred at 105° C. for 24 hours. During the first 8 hours, water was repeatedly added in small portions each hour. Upon cooling to room temperature, the reaction mixture was concentrated under reduced pressure. The residue was triturated with a diethyl ether and dichloromethane mixture. The resulting precipitate was collected by vacuum filtration, washed with diethyl ether, and dried under vacuum to give 7-(trifluoromethyl)-2H,3H,7H-pyrrolo[2,3-d]pyrimidin-2-one (7.12 g, 32.2 mmol, 89%) as a beige solid.

¹H NMR (400 MHz, DMSO-d6) δ ppm 8.40 (s, 1H), 7.45 (d, J=4.2 Hz, 1H), 6.55 (d, J=4.2 Hz, 1H).

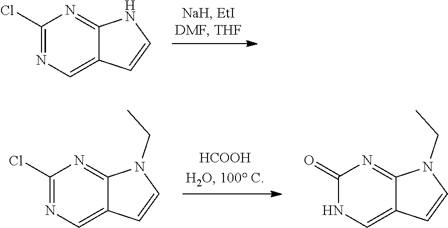

### 2-chloro-7-ethyl-7H-pyrrolo[2,3-d]pyrimidine

At 0° C., sodium hydride (1.25 g, 31.3 mmol, 1.2 eq, 60% in mineral oil) was added to a solution of 2-chloro-7H-pyrrolo[2,3-d]pyrimidine (4.0 g, 26.0 mmol, 1.0 eq) in a mixture of anhydrous THF (40.0 mL) and anhydrous DMF (40.0 mL). The resulting solution was stirred at 0° C. for 15 minutes. Then, iodoethane (4.88 g, 31.3 mmol, 1.2 eq) was added dropwise. The reaction mixture was stirred at room temperature for 16 hours. The reaction was diluted with water and extracted three times with diethyl ether. The combined organic layers were washed with brine, dried over sodium sulfate, and concentrated under reduced pressure. The resulting residue was purified by silica gel column chromatography (5-15% EtOAc in hexanes) to afford 2-chloro-7-ethyl-7H-pyrrolo[2,3-d]pyrimidine (3.86 g, 21.3 mmol, 78%) as a solid.

¹H NMR (300 MHz, DMSO-d6) δ ppm 8.90 (s, 1H), 7.72 (d, J=3.6 Hz, 1H), 6.68 (d, J=3.6 Hz, 1H), 4.24 (q, J=7.3 Hz, 2H), 1.37 (t, J=7.3 Hz, 3H).

### 7-ethyl-2H,3H,7H-pyrrolo[2,3-d]pyrimidin-2-one

2-Chloro-7-ethyl-7H-pyrrolo[2,3-d]pyrimidine (3.86 g, 20.4 mmol, 1.0 eq) was dissolved in a mixture of formic acid (23.1 mL, 612 mmol, 30.0 eq) and water (1.84 mL, 102 mmol, 5.0 eq). The reaction mixture was stirred at 100° C. for 16 hours. Additional water (5.0 eq) was added, and the reaction was stirred at 100° C. for an additional 5 days. After cooling to 0° C., ethyl acetate was added, and a precipitate was formed. The resulting precipitate was collected by vacuum filtration, washed with ethyl acetate, and dried under vacuum to afford 7-ethyl-2H,3H,7H-pyrrolo[2,3-d]pyrimidin-2-one (2.28 g, 14.0 mmol, 68%) as a brown solid.

¹H NMR (300 MHz, DMSO-d6) δ ppm 8.74 (s, 1H), 7.45 (d, J=3.8 Hz, 1H), 6.63 (d, J=3.8 Hz, 1H), 4.13 (q, J=7.2 Hz, 2H), 1.31 (t, J=7.2 Hz, 3H).

Additional alkyl 7-alkyl-2H,3H,7H-pyrrolo[2,3-d]pyrimidin-2-ones were synthesized in a similar manner to 7-ethyl-2H,3H,7H-pyrrolo[2,3-d]pyrimidin-2-one.

### Synthesis of Compound 186.

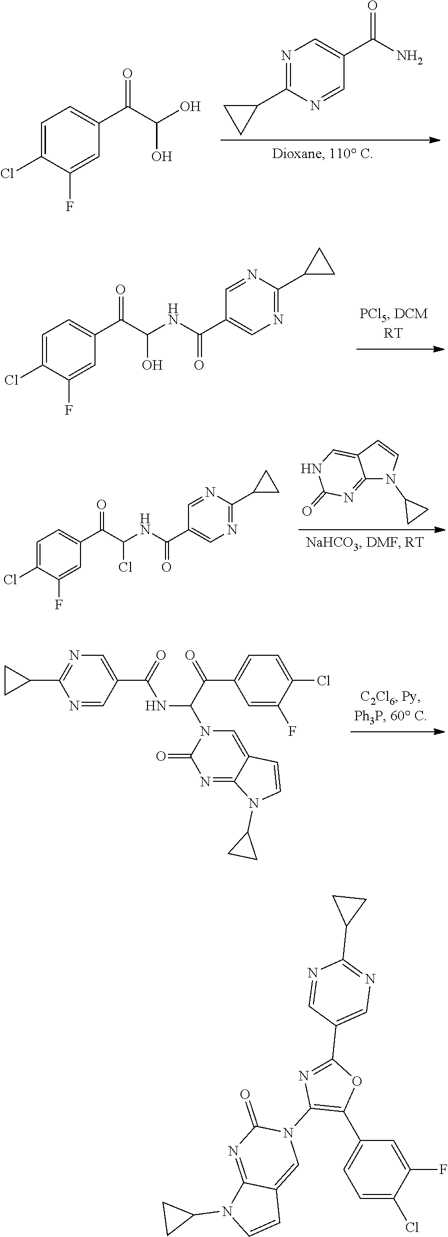

### N-[2-(4-chloro-3-fluorophenyl)-1-hydroxy-2-oxoethyl]-2-cyclopropylpyrimidine-5-carboxamide

2-Cyclopropylpyrimidine-5-carboxamide (0.80 g, 4.81 mmol, 1.0 eq) and 4-chloro-3-fluorophenylglyoxal monohydrate (0.983 g, 4.81 mmol, 1.0 eq) were suspended in dioxane (4.0 mL) and the reaction mixture was stirred for 5 hours at 100° C. The dioxane was evaporated, and the obtained crude material was triturated with ethyl acetate. The resulting precipitate was collected by vacuum filtration, washed with cold ethyl acetate, and dried under vacuum to give N-[2-(4-chloro-3-fluorophenyl)-1-hydroxy-2-oxoethyl]-2-cyclopropylpyrimidine-5-carboxamide (1.02 g, 2.77 mmol, 58%) as white solid.

¹H NMR (300 MHz, DMSO-d6) δ ppm 9.71 (d, J=7.7 Hz, 1H), 9.07-8.99 (m, 2H), 7.95 (dd, J=10.0, 1.8 Hz, 1H), 7.88-7.74 (m, 2H), 6.45 (d, J=7.7 Hz, 1H), 2.32-2.22 (m, 1H), 1.17-1.03 (m, 4H).

### N-[1-chloro-2-(4-chloro-3-fluorophenyl)-2-oxoethyl]-2-cyclopropylpyrimidine-5-carboxamide

N-[2-(4-Chloro-3-fluorophenyl)-1-hydroxy-2-oxoethyl]-2-cyclopropylpyrimidine-5-carboxamide (0.40 g, 1.09 mmol, 1.0 eq) was dissolved in dichloromethane (8.0 mL). Phosphorus pentachloride (0.249 g, 1.20 mmol, 1.1 eq) was added and the resulting mixture was stirred at room temperature for 16 hours. The reaction mixture was diluted with n-pentane. The resulting precipitate was collected by vacuum filtration, washed with pentane, washed with diethyl ether, and dried under vacuum to afford N-[1-chloro-2-(4-chloro-3-fluorophenyl)-2-oxoethyl]-2-cyclopropylpyrimidine-5-carboxamide (0.439 g, 1.07 mmol, 99%) as a white solid.

¹H NMR (300 MHz, DMSO-d6) δ ppm 9.69 (d, J=7.7 Hz, 1H), 9.01 (s, 2H), 7.95 (dd, J=10.1, 1.8 Hz, 1H), 7.87-7.75 (m, 2H), 6.46 (d, J=7.7 Hz, 1H), 2.32-2.22 (m, 1H), 1.17-1.02 (m, 4H).

N-[2-(4-chloro-3-fluorophenyl)-1-{7-cyclopropyl-2-oxo-2H,3H,7H-pyrrolo[2,3-d]pyrimidin-3-yl}-2-oxoethyl]-2-cyclopropylpyrimidine-5-carboxamide

7-Cyclopropyl-2H,3H,7H-pyrrolo[2,3-d]pyrimidin-2-one (0.192 g, 1.07 mmol, 1.0 eq) was added to a solution of sodium bicarbonate (0.451 g, 5.37 mmol, 5.0 eq) in DMF (8.78 mL) and the reaction mixture was stirred for 10 minutes at 0° C. Then, N-[1-chloro-2-(4-chloro-3-fluorophenyl)-2-oxoethyl]-2-cyclopropylpyrimidine-5-carboxamide (0.439 g, 1.07 mmol, 1.0 eq) was added, and the reaction was mixture was stirred at room temperature for 16 hours. The reaction mixture was diluted with ethyl acetate and washed with brine three times. The organic solution was dried over Na₂SO₄ and concentrated under reduced pressure. The residue was purified by silica gel column chromatography (0-5% MeOH in DCM) to afford N-[2-(4-chloro-3-fluorophenyl)-1-{7-cyclopropyl-2-oxo-2H,3H,7H-pyrrolo[2,3-d]pyrimidin-3-yl}-2-oxoethyl]-2-cyclopropylpyrimidine-5-carboxamide (0.333 g, 0.657 mmol, 61%) as a beige solid.

UPLC-MS: 2.78 min, [M+H]⁺=507.85, [M−H]⁻=505.00, 95%

### 3-[5-(4-chloro-3-fluorophenyl)-2-(2-cyclopropylpyrimidin-5-yl)-1,3-oxazol-4-yl]-7-cyclopropyl-2H,3H,7H-pyrrolo[2,3-d]pyrimidin-2-one. Compound 186

N-[2-(4-Chloro-3-fluorophenyl)-1-{7-cyclopropyl-2-oxo-2H,3H,7H-pyrrolo[2,3-d]pyrimidin-3-yl}-2-oxoethyl]-2-cyclopropylpyrimidine-5-carboxamide (0.333 g, 0.623 mmol, 1.0 eq) was suspended in anhydrous MeCN (6.66 mL). Then, hexachloroethane (0.295 g, 1.25 mmol, 2.0 eq) and triphenylphosphine (0.327 g, 1.25 mmol, 2.0 eq) were added. The resulting solution was stirred for 10 minutes at room temperature before pyridine (0.201 mL, 2.50 mmol, 4.0 eq) was added. The reaction was then stirred at 60° C. for 16 hours. The crude reaction was cooled to room temperature and concentrated under reduced pressure. The residue was then suspended in methanol and stirred for 10 minutes. The formed precipitate was collected by vacuum filtration, washed with methanol and diethyl ether, and dried under vacuum to afford 3-[5-(4-chloro-3-fluorophenyl)-2-(2-cyclopropylpyrimidin-5-yl)-1,3-oxazol-4-yl]-7-cyclopropyl-2H,3H,7H-pyrrolo[2,3-d]pyrimidin-2-one (0.12 g, 0.244 mmol, 39%).

LCMS: 2.44 min, [M+H]⁺=489.12, 99.40%, @220 nm

¹H NMR (300 MHz, DMSO-d6) δ ppm 9.36 (s, 2H), 8.59 (s, 1H), 7.80 (dd, J=10.4, 1.8 Hz, 1H), 7.73 (t, J=8.1 Hz, 1H), 7.35-7.25 (m, 1H), 7.23 (d, J=3.9 Hz, 1H), 6.32 (d, J=3.9 Hz, 1H), 2.37-2.31 (m, 1H), 1.23-1.10 (m, 4H), 1.06-0.95 (m, 4H).

Additional Compounds Synthesized in an Analogous Manner to Compound 186:

| Compound | Exact Mass | LCMS Found | Assignment |
|---|---|---|---|
| 187 | 406.34 | 406.9 | (M + H)⁺ |
| 188 | 422.37 | 423.1 | (M + H)⁺ |
| 189 | 430.42 | 431.1 | (M + H)⁺ |
| 190 | 434.86 | 435.1 | (M + H)⁺ |
| 191 | 434.86 | 435.2 | (M + H)⁺ |
| 192 | 438.82 | 439.1 | (M + H)⁺ |
| 193 | 444.44 | 445.0 | (M + H)⁺ |
| 194 | 446.87 | 447.1 | (M + H)⁺ |
| 195 | 448.88 | 448.9 | (M + H)⁺ |
| 196 | 452.85 | 453.1 | (M + H)⁺ |
| 197 | 460.89 | 461.2 | (M + H)⁺ |
| 198 | 462.43 | 463.2 | (M + H)⁺ |
| 199 | 464.86 | 465.1 | (M + H)⁺ |
| 200 | 474.8 | 475.0 | (M + H)⁺ |
| 201 | 474.92 | 474.9 | (M + H)⁺ |
| 202 | 476.94 | 477.2 | (M + H)⁺ |
| 203 | 478.88 | 479.2 | (M + H)⁺ |
| 204 | 482.85 | 483.0 | (M + H)⁺ |
| 205 | 488.83 | 489.0 | (M + H)⁺ |
| 206 | 492.46 | 492.9 | (M + H)⁺ |
| 207 | 496.87 | 497.2 | (M + H)⁺ |
| 208 | 520.39 | 521.1 | (M + H)⁺ |

### Synthesis of Compound 209.

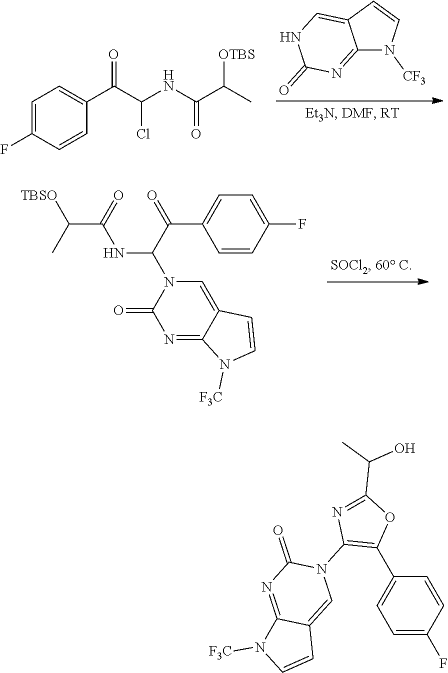

2-[(tert-butyldimethylsilyl)oxy]-N-[1-chloro-2-(4-fluorophenyl)-2-oxoethyl]propenamide was prepared in similar fashion to N-[1-chloro-2-(4-chloro-3-fluorophenyl)-2-oxoethyl]-2-cyclopropylpyrimidine-5-carboxamide shown above.

### 2-[(tert-butyldimethylsilyl)oxy]-N-[2-(4-fluorophenyl)-2-oxo-1-[2-oxo-7-(trifluoromethyl)-2H,3H,7H-pyrrolo[2,3-d]pyrimidin-3-yl]ethyl]propanamide

7-(Trifluoromethyl)-2H,3H,7H-pyrrolo[2,3-d]pyrimidin-2-one (0.15 g, 0.731 mmol, 1.0 eq) and triethylamine (0.51 mL, 3.66 mmol, 5.0 eq) were dissolved in DMF (3.75 mL) at 0° C. and stirred for 30 minutes at the same temperature. Then, 2-[(tert-butyldimethylsilyl)oxy]-N-[1-chloro-2-(4-fluorophenyl)-2-oxoethyl]propanamide (0.60 g, 1.49 mmol, 2.04 eq) was added in portions over 30 minutes. The reaction was stirred at room temperature for 16 hours. The reaction mixture was diluted with ethyl acetate and washed with brine three times. The organic solution was dried over Na₂SO₄ and concentrated under reduced pressure. The residue was purified by silica gel column chromatography (0-5% MeOH in DCM) to give 2-[(tert-butyldimethylsilyl)oxy]-N-[2-(4-fluorophenyl)-2-oxo-1-[2-oxo-7-(trifluoromethyl)-2H,3H,7H-pyrrolo[2,3-d]pyrimidin-3-yl]ethyl]propanamide (0.30 g, 0.433 mmol, 59%) as a white solid.

UPLC-MS: 4.12 min, [M+H]⁺=540.70, 78%

### 3-[5-(4-fluorophenyl)-2-(1-hydroxyethyl)-1,3-oxazol-4-yl]-7-(trifluoromethyl)-2H,3H,7H-pyrrolo[2,3-d]pyrimidin-2-one. Compound 209

2-[(Tert-butyldimethylsilyl)oxy]-N-[2-(4-fluorophenyl)-2-oxo-1-[2-oxo-7-(trifluoromethyl)-2H,3H,7H-pyrrolo[2,3-d]pyrimidin-3-yl]ethyl]propanamide (0.30 g, 0.433 mmol, 1.0 eq) was dissolved in thionyl chloride (1.25 mL, 17.3 mmol, 40.0 eq) and the mixture was heated to 60° C. for 5 hours. The reaction was cooled to room temperature and concentrated under reduced pressure. Methanol was added to the residue and a precipitate formed. This solid was collected by vacuum filtration, washed with cold methanol, and dried under vacuum. The crude solid was purified by prep-HPLC (C18 column, 10-100% MeCN in water with 0.1% TFA) to give 3-[5-(4-fluorophenyl)-2-(1-hydroxyethyl)-1,3-oxazol-4-yl]-7-(trifluoromethyl)-2H,3H,7H-pyrrolo[2,3-d]pyrimidin-2-one (0.008 g, 0.019 mmol, 4%) as an orange solid.

LCMS: 5.70, [M+H]⁺=408.90, 99% @ 220 nm

¹H NMR (300 MHz, DMSO-d6) δ ppm 8.78 (s, 1H), 7.60 (d, J=4.3 Hz, 1H), 7.57-7.42 (m, 2H), 7.39-7.21 (m, 2H), 6.62 (d, J=4.3 Hz, 1H), 4.89 (q, J=6.6 Hz, 1H), 1.52 (d, J=6.6 Hz, 3H).

Additional Compounds Prepared in an Analogous Manner to Compound 209:

| Compound | Exact Mass | LCMS Found | Assignment |
|---|---|---|---|
| 210 | 418.35 | 418.9 | (M + H)⁺ |
| 211 | 392.31 | 393.0 | (M + H)⁺ |
| 212 | 404.32 | 405.0 | (M + H)⁺ |
| 293 | 482.11 | 483.1 | (M + H)⁺ |

Synthesis of Compound 213.

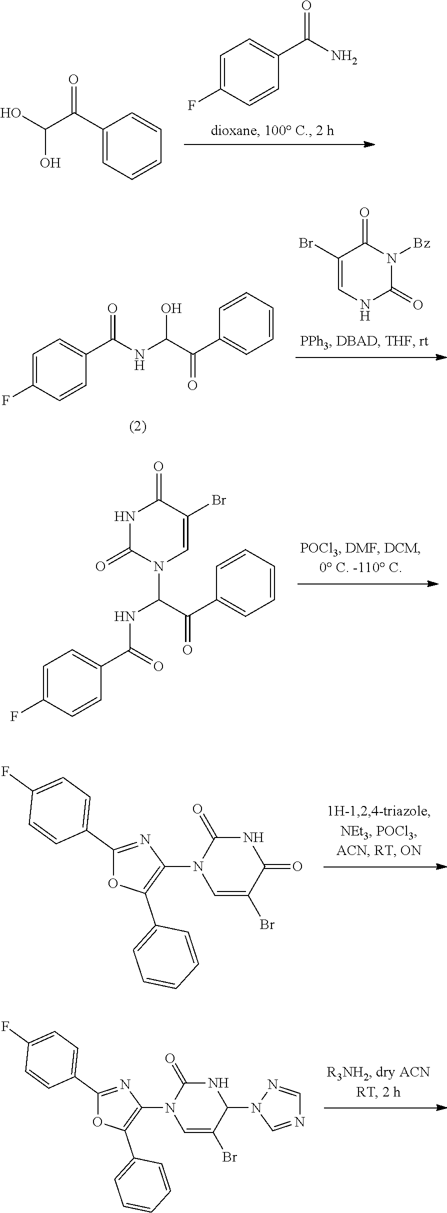

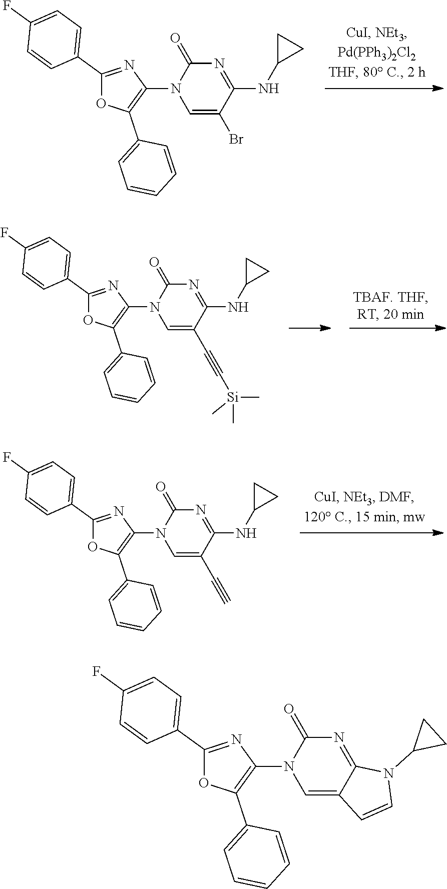 [SMILES: O=c1nc(NC2CC2)c(Br)cn1-c1nc(-c2ccc(F)cc2)oc1-c1ccccc1 ; C[Si](C)(C)C#Cc1cn(-c2nc(-c3ccc(F)cc3)oc2-c2ccccc2)c(=O)nc1NC1CC1 ; C#Cc1cn(-c2nc(-c3ccc(F)cc3)oc2-c2ccccc2)c(=O)nc1NC1CC1 ; O=c1nc2c(ccn2C2CC2)cn1-c1nc(-c2ccc(F)cc2)oc1-c1ccccc1]

### 4-fluoro-N-(1-hydroxy-2-oxo-2-phenylethyl)benzamide

2,2-Dihydroxy-1-phenylethanone (13.5 g, 79.1 mmol, 1.1 eq) and 4-fluorobenzamide (10.0 g, 71.9 mmol, 1.0 eq) were suspended in dioxane (200 mL) and the mixture was heated to 100° C. for 5 hours. Upon cooling to room temperature, the dioxane was evaporated. The resulting residue was triturated in a mixture of hexanes in ethyl acetate (2/1, 10.0 volumes). The resulting solid was collected by vacuum filtration, washed with cold hexanes and diethyl ether, and dried under vacuum to give 4-fluoro-N-(1-hydroxy-2-oxo-2-phenylethyl)benzamide (13.5 g, 49.4 mmol, 67%).

UPLC-MS: RT=2.89 min, [M+H]⁺=274.05, [M−H]⁺=272.00, 100% @254 nm.

### N-(1-(5-bromo-2,4-dioxo-3,4-dihydropyrimidin-1(2H)-yl)-2-oxo-2-phenylethyl)-4-fluorobenzamide

3-Benzoyl-5-bromo-1,2,3,4-tetrahydropyrimidine-2,4-dione (2.0 g, 6.78 mmol, 1.1 eq), 4-fluoro-N-(1-hydroxy-2-oxo-2-phenylethyl)benzamide (1.68 g, 6.16 mmol, 1.0 eq), and triphenylphosphine (1.78 g, 0.68 mmol, 1.1 eq) were placed in a flask and suspended in dry toluene. The toluene was evaporated under reduced pressure. This process was repeated 3 times. Anhydrous THF (50.5 mL) was then added under an argon atmosphere followed by di-tert-butyl azodicarboxylate (1.84 g, 8.01 mmol, 1.3 eq). The resulting mixture was stirred at room temperature for 16 hours. The solvent was removed under reduced pressure. The crude material was dissolved in methanol and cooled in an ice-bath. The formed precipitate was collected by vacuum filtration, washed with methanol followed by diethyl ether, and dried under vacuum to give N-(1-(5-bromo-2,4-dioxo-3,4-dihydropyrimidin-1(2H)-yl)-2-oxo-2-phenylethyl)-4-fluorobenzamide (2.50 g, 5.60 mmol, 91%).

UPLC-MS: RT=3.85 min, [M+H]⁺=447.65, 90% @254 nm.

### 5-bromo-1-(2-(4-fluorophenyl)-5-phenyloxazol-4-yl)pyrimidine-2,4(1H,3H)-dione

Dimethylformamide (1.22 mL, 16.8 mmol, 3.0 eq) was added dropwise to phosphorus(V) oxychloride (3.13 mL, 33.6 mmol, 6.0 eq) at 0° C. and the mixture was stirred for 10 minutes. The mixture was then warmed to room temperature and N-(1-(5-bromo-2,4-dioxo-3,4-dihydropyrimidin-1(2H)-yl)-2-oxo-2-phenylethyl)-4-fluorobenzamide (2.50 g, 5.60 mmol, 1.0 eq) in dichloromethane (12.0 mL) was added. The mixture was heated to 110° C. for 16 hours. Upon cooling to room temperature, the formed precipitate was collected by vacuum filtration, washed with dimethylformamide followed by diethyl ether, and dried under vacuum to afford 5-bromo-1-(2-(4-fluorophenyl)-5-phenyloxazol-4-yl)pyrimidine-2,4(1H,3H)-dione (1.40 g, 3.28 mmol, 58%).

UPLC-MS: RT=3.65 min, [M+H]⁺=428.75, 91% @254 nm.

### 5-bromo-1-(2-(4-fluorophenyl)-5-phenyloxazol-4-yl)-4-(1H-1,2,4-triazol-1-yl)pyrimidin-2(1H)-one

5-Bromo-1-(2-(4-fluorophenyl)-5-phenyloxazol-4-yl)pyrimidine-2,4(1H,3H)-dione (1.40 g, 3.28 mmol, 1.0 eq) was suspended in a mixture of anhydrous MeCN (42.0 mL) and anhydrous DCM (7.0 mL). Then, triethylamine (5.48 mL, 39.3 mmol, 12.0 eq) and 1,2,4-triazole (1.81 g, 26.2 mmol, 8.0 eq) were added followed by the dropwise addition of phosphorus(V) oxychloride (1.65 mL, 6.56 mmol, 2.0 eq). The reaction mixture was stirred at room temperature for 16 hours. The formed precipitate was collected by vacuum filtration, washed with acetonitrile, and dried under vacuum to afford 5-bromo-1-(2-(4-fluorophenyl)-5-phenyloxazol-4-yl)-4-(1H-1,2,4-triazol-1-yl)pyrimidin-2(1H)-one (1.56 g, 3.28 mmol, 100%).

UPLC-MS: RT=4.30 min, mass not detected, 98% @254 nm.

### 5-bromo-4-(cyclopropylamino)-1-(2-(4-fluorophenyl)-5-phenyloxazol-4-yl)pyrimidin-2(1H)-one

To a suspension of 5-bromo-1-(2-(4-fluorophenyl)-5-phenyloxazol-4-yl)-4-(1H-1,2,4-triazol-1-yl)pyrimidin-2(1H)-one (0.50 g, 1.04 mmol, 1.0 eq) in anhydrous MeCN (10.0 mL) was added cyclopropylamine (0.11 mL, 1.56 mmol, 1.5 eq). The reaction mixture was stirred at room temperature for 2 hours. The resulting precipitate was collected by vacuum filtration and washed with methanol and acetonitrile. The solid was then dried under vacuum to afford 5-bromo-4-(cyclopropylamino)-1-(2-(4-fluorophenyl)-5-phenyloxazol-4-yl)pyrimidin-2(1H)-one (0.30 g, 0.64 mmol, 63%).

UPLC-MS: RT=3.65 min, [M+H]⁺=468.00, 95% @254 nm.

### 4-(cyclopropylamino)-1-(2-(4-fluorophenyl)-5-phenyloxazol-4-yl)-5-((trimethylsilyl)ethynyl)pyrimidin-2(1H)-one

To a solution of 5-bromo-4-(cyclopropylamino)-1-(2-(4-fluorophenyl)-5-phenyloxazol-4-yl)pyrimidin-2(1H)-one (0.30 g, 0.64 mmol, 1.0 eq) in anhydrous THF (7.50 mL) were added bis(triphenylphosphine)palladium(II) dichloride (0.013 g, 0.02 mmol, 0.03 eq), copper iodide (0.004 g, 0.02 mmol, 0.03 eq), triethylamine (0.27 mL, 1.92 mmol, 3.0 eq), and trimethylsilylacetylene (0.89 mL, 6.39 mmol, 10.0 eq) under argon atmosphere. The reaction was stirred in a sealed reaction tube for 16 hours at 80° C. The reaction was cooled to room temperature, diluted with ethyl acetate, and filtered through a celite pad eluting with ethyl acetate. The filtrate was concentrated under reduced pressure. The residue was purified by silica gel column chromatography (0-5% MeOH in DCM) to afford 4-(cyclopropylamino)-1-(2-(4-fluorophenyl)-5-phenyloxazol-4-yl)-5-((trimethylsilyl)ethynyl)pyrimidin-2(1H)-one (0.13 g, 0.27 mmol, 42%).

UPLC-MS: RT=4.46 min, [M+H]⁺=485.30, 70% @254 nm.

### 4-(cyclopropylamino)-5-ethynyl-1-(2-(4-fluorophenyl)-5-phenyloxazol-4-yl)pyrimidin-2(1H)-one

4-(Cyclopropylamino)-1-(2-(4-fluorophenyl)-5-phenyloxazol-4-yl)-5-((trimethylsilyl)ethynyl)pyrimidin-2(1H)-one (0.10 g, 0.21 mmol, 1.0 eq) was dissolved in tetrahydrofuran (4.0 mL) at 0° C. Tetrabutylammonium fluoride trihydrate (0.07 g, 1.05 eq) was added to the solution in one portion, and the reaction was stirred at temperature for 1 hour. The reaction was quenched with water and extracted with ethyl acetate. The combined organic layers were dried over sodium sulfate and concentrated under reduced pressure. The crude material was purified by silica gel column chromatography (0-3% MeOH in DCM). The resulting impure product was triturated with diethyl ether to afford 4-(cyclopropylamino)-5-ethynyl-1-(2-(4-fluorophenyl)-5-phenyloxazol-4-yl)pyrimidin-2(1H)-one (0.13 g, 0.27 mmol, 100%).

UPLC-MS: RT=43.58 min, [M+H]⁺=413.45, [M−H]⁺=410.65, 85% @254 nm.

### 7-cyclopropyl-3-(2-(4-fluorophenyl)-5-phenyloxazol-4-yl)-3,7-dihydro-2H-pyrrolo[2,3-d]pyrimidin-2-one. Compound 213

4-(Cyclopropylamino)-5-ethynyl-1-(2-(4-fluorophenyl)-5-phenyloxazol-4-yl)pyrimidin-2(1H)-one (0.13 g, 0.27 mmol, 1.0 eq) and copper iodide (0.51 g, 0.27 mmol, 1.0 eq) were placed in a microwave vial. Anhydrous DMF (2.60 mL) and triethylamine (2.60 mL) were added. The reaction mixture was purged with argon for 5 minutes. Then, the reaction tube was sealed and heated in the microwave reactor at 120° C. for 15 minutes. The reaction was cooled to room temperature, diluted with ethyl acetate, and filtered through a celite pad eluting with additional ethyl acetate. The filtrate was washed with brine (3 times). The organic solution was dried over sodium sulfate and concentrated under reduced pressure. The crude material was purified by preparative TLC (eluting with 100% DCM, 2% MeOH in DCM, and 3% MeOH in DCM) to afford 7-cyclopropyl-3-(2-(4-fluorophenyl)-5-phenyloxazol-4-yl)-3,7-dihydro-2H-pyrrolo[2,3-d]pyrimidin-2-one (0.013 g, 0.03 mmol, 10%).

LCMS: 6.05 min, [M+H]⁺=413.16, 99.69% @ 220 nm

¹H NMR (300 MHz, Chloroform-d) δ ppm 8.12 (dd, J=8.8, 5.3 Hz, 2H), 7.93 (s, 1H), 7.64-7.56 (m, 2H), 7.44-7.36 (m, 2H), 7.21 (t, J=8.6 Hz, 2H), 6.89 (d, J=4.0 Hz, 1H), 6.16 (d, J=4.0 Hz, 1H), 3.38-3.32 (m, 1H), 1.13 (t, J=6.2 Hz, 2H), 1.05 (d, J=6.0 Hz, 2H).

Compounds Synthesized in an Analogous Manner to Compound 213:

| Compound | Exact Mass | LCMS Found | Assignment |
|---|---|---|---|
| 214 | 386.12 | 387.1 | (M + H)⁺ |
| 215 | 394.14 | 395.5 | (M + H)⁺ |

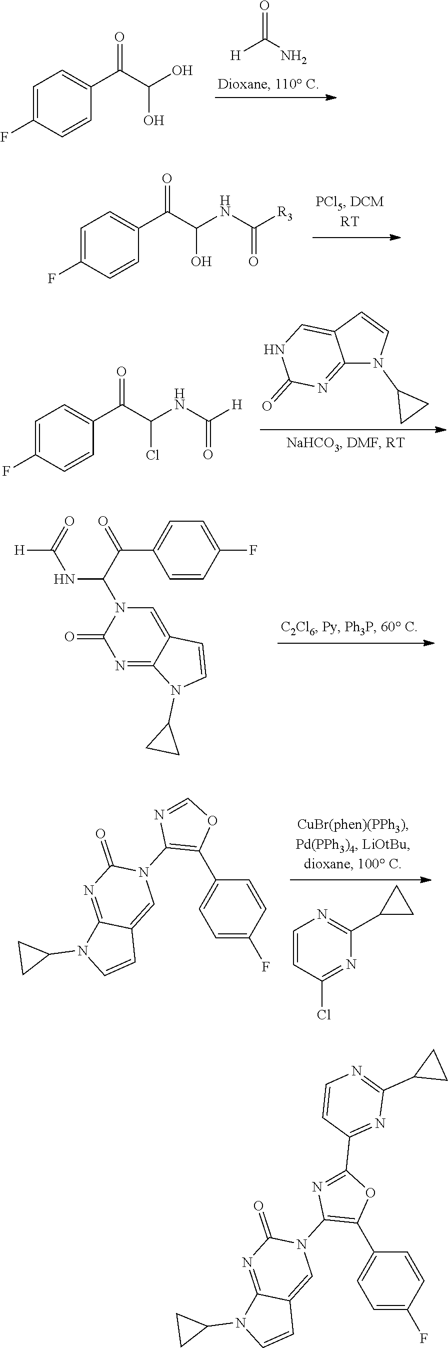

### Synthesis of Compound 216.

### N-[2-(4-fluorophenyl)-1-hydroxy-2-oxoethyl]formamide

Formamide (1.41 mL, 35.3 mmol, 3.0 eq) and 4-fluorophenylglyoxal hydrate (2.0 g, 11.8 mmol, 1.0 eq) were dissolved in dioxane (24.4 mL). The resulting mixture was heated to 100° C. and stirred for 3 hours. Upon cooling to room temperature, the dioxane was removed under reduced pressure. The residue was dissolved in ethyl acetate and washed with saturated aqueous sodium bicarbonate 2 times. The organic layer was dried over sodium sulfate and concentrated under reduced pressure. The obtained material was triturated with a mixture of ethyl acetate and hexane (3 to 7) to give N-[2-(4-fluorophenyl)-1-hydroxy-2-oxoethyl]formamide (1.66 g, 6.74 mmol, 57%).

¹H NMR (300 MHz, DMSO-d6) δ ppm 8.98 (d, J=8.7 Hz, 1H), 8.33-7.94 (m, 3H), 7.45-7.21 (m, 2H), 6.78 (d, J=7.3 Hz, 1H), 6.68-6.22 (m, 1H).

### N-[1-chloro-2-(4-fluorophenyl)-2-oxoethyl]formamide

N-[2-(4-Fluorophenyl)-1-hydroxy-2-oxoethyl]formamide (1.66 g, 6.74 mmol, 1.0 eq) was dissolved in DCM (33.2 mL). Phosphorus pentachloride (1.54 g, 7.41 mmol, 1.1 eq) was added and the resulting mixture was stirred for 18 hours at room temperature. Pentane was added directly to the reaction mixture and a precipitate formed. This material was collected by vacuum filtration, washed with pentane, and dried under vacuum to give N-[1-chloro-2-(4-fluorophenyl)-2-oxoethyl]formamide (1.54 g, 5.73 mmol, 85%).

¹H NMR (300 MHz, DMSO-d6) δ ppm 8.98 (d, J=8.7 Hz, 1H), 8.36-7.77 (m, 3H), 7.54-6.92 (m, 2H), 6.36 (dd, J=8.7, 0.8 Hz, 1H).

### N-(1-{7-cyclopropyl-2-oxo-2H,3H,7H-pyrrolo[2,3-d]pyrimidin-3-yl}-2-(4-fluorophenyl)-2-oxoethyl)formamide

7-Cyclopropyl-2H,3H,7H-pyrrolo[2,3-d]pyrimidin-2-one (1.09 g, 5.73 mmol, 1.0 eq) and sodium bicarbonate (3.37 g, 40.1 mmol, 7.0 eq) were suspended in DMF (30.9 mL). The resulting mixture was cooled to 0° C. and stirred at for 10 minutes. Then, N-[1-chloro-2-(4-fluorophenyl)-2-oxoethyl]formamide (1.54 g, 5.73 mmol, 1.0 eq) was added. The mixture was then stirred for 24 hours at room temperature. The solvent was removed under reduced pressure. The residue was taken up in DCM and washed with brine 2 times. The organic layer was dried over sodium sulfate and concentrated under reduced pressure. The crude product was then triturated with ethyl acetate, collected by vacuum filtration, washed with cold ethyl acetate, and dried under vacuum to give N-(1-{7-cyclopropyl-2-oxo-2H,3H,7H-pyrrolo[2,3-d]pyrimidin-3-yl}-2-(4-fluorophenyl)-2-oxoethyl)formamide (1.29 g, 3.53 mmol, 62%).

¹H NMR (300 MHz, DMSO-d6) δ ppm 9.87-9.39 (m, 1H), 8.65 (s, 1H), 8.24 (d, J=1.0 Hz, 1H), 8.09-7.73 (m, 2H), 7.36 (t, J=8.8 Hz, 3H), 7.14 (d, J=4.0 Hz, 1H), 6.36 (d, J=3.9 Hz, 1H), 3.24 (tt, J=6.2, 4.2 Hz, 1H), 0.91 (ddt, J=6.8, 4.7, 1.8 Hz, 4H).

### 7-cyclopropyl-3-[5-(4-fluorophenyl)-1,3-oxazol-4-yl]-2H,3H,7H-pyrrolo[2,3-cl]pyrimidin-2-one

N-(1-{7-Cyclopropyl-2-oxo-2H,3H,7H-pyrrolo[2,3-d]pyrimidin-3-yl}-2-(4-fluorophenyl)-2-oxoethyl)formamide (1.29 g, 3.53 mmol, 1.0 eq) was suspended in anhydrous MeCN (25.8 mL). Hexachloroethane (1.67 g, 7.07 mmol, 2.0 eq) and triphenylphosphine (1.85 g, 7.07 mmol, 2.0 eq) were added, and the solution was stirred for 10 minutes at room temperature. Anhydrous pyridine (1.14 mL, 14.1 mmol, 4.0 eq) was added, and the reaction was stirred at 60° C. for 24 hours. Upon cooling to room temperature, the solvent was removed under reduced pressure. The crude product was purified by silica gel column chromatography (0-5% MeOH in DCM) to give 7-cyclopropyl-3-[5-(4-fluorophenyl)-1,3-oxazol-4-yl]-2H,3H,7H-pyrrolo[2,3-d]pyrimidin-2-one (0.394 g, 1.17 mmol, 33%).

¹H NMR (300 MHz, DMSO-d6) δ ppm 8.64 (s, 1H), 8.52 (s, 1H), 7.52-7.43 (m, 2H), 7.41-7.24 (m, 2H), 7.21 (d, J=4.0 Hz, 1H), 6.28 (d, J=4.0 Hz, 1H), 1.04-0.94 (m, 4H).

UPLC-MS: 1.89 min, [M+H]⁺=336.85, 93%

### 7-cyclopropyl-3-[2-(2-cyclopropylpyrimidin-4-yl)-5-(4-fluorophenyl)-1,3-oxazol-4-yl]-2H,3H,7H-pyrrolo[2,3-d]pyrimidin-2-one. Compound 216

Under inert atmosphere, 7-cyclopropyl-3-[5-(4-fluorophenyl)-1,3-oxazol-4-yl]-2H,3H,7H-pyrrolo[2,3-d]pyrimidin-2-one (0.10 g, 0.297 mmol, 1.0 eq), 4-chloro-2-cyclopropylpyrimidine (0.069 g, 0.446 mmol, 1.5 eq), bromo(1,10-phenanthroline)(triphenylphosphine)copper(I) (0.017 g, 0.03 mmol, 0.1 eq), and tetrakis(triphenylphosphine)palladium (0.034 g, 0.03 mmol, 0.1 eq) were dissolved in anhydrous dioxane (1.0 mL). The mixture was purged with argon for 10 minutes before lithium tert-butoxide (0.173 g, 0.595 mmol, 2.0 eq) was added. The reaction tube was sealed, and the mixture was stirred for 3 hours at 100° C. The reaction mixture was allowed to cool to room temperature and diluted with dichloromethane. Brine was added and the layers were separated. The aqueous layer was extracted with dichloromethane 3 times. The combined organic layers were dried over sodium sulfate and concentrated under reduced pressure to give crude product. This material was purified by silica gel column chromatography (0-5% MeOH in DCM) to give 7-cyclopropyl-3-[2-(2-cyclopropylpyrimidin-4-yl)-5-(4-fluorophenyl)-1,3-oxazol-4-yl]-2H,3H,7H-pyrrolo[2,3-d]pyrimidin-2-one (0.023 g, 0.048 mmol, 16%) as on off-white solid.

LCMS: 2.830 min, [M+H]⁺=455.17, 95.1% @ 220 nm

¹H NMR (300 MHz, DMSO-d6) δ ppm 8.90 (d, J=5.1 Hz, 1H), 8.61 (s, 1H), 7.96 (d, J=5.1 Hz, 1H), 7.68-7.56 (m, 2H), 7.41 (t, J=8.9 Hz, 2H), 7.24 (d, J=4.0 Hz, 1H), 6.32 (d, J=3.9 Hz, 1H), 3.44-3.33 (m, 1H), 2.40-2.30 (m, 1H), 1.14 (t, J=6.1 Hz, 4H), 1.01 (d, J=5.6 Hz, 4H).

| | Exact | LCMS | |
|---|---|---|---|
| Compound | Mass | Found | Assignment |
| 217 | 468.17 | 469.2 | (M + H)⁺ |
| 218 | 476.12 | 477.1 | (M + H)⁺ |
| 219 | 481.12 | 482.2 | (M + H)⁺ |
| 220 | 482.11 | 482.9 | (M + H)⁺ |
| 221 | 487.12 | 488.1 | (M + H)⁺ |
| 222 | 487.12 | 488.1 | (M + H)⁺ |
| 223 | 490.13 | 491.1 | (M + H)⁺ |
| 224 | 524.1 | 524.8 | (M + H)⁺ |

### Synthesis of Compound 225.

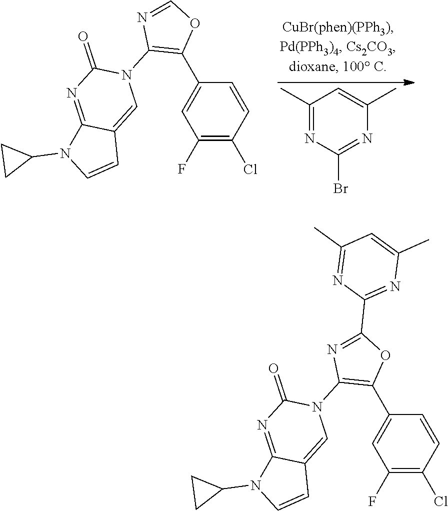

3-[5-(4-chloro-3-fluorophenyl)-1,3-oxazol-4-yl]-7-cyclopropyl-2H,3H,7H-pyrrolo[2,3-d]pyrimidin-2-one was synthesized in a similar fashion to 7-cyclopropyl-3-[5-(4-fluorophenyl)-1,3-oxazol-4-yl]-2H,3H,7H-pyrrolo[2,3-d]pyrimidin-2-one as shown above.

### 3-[5-(4-chloro-3-fluorophenyl)-2-(4,6-dimethylpyrimidin-2-yl)-1,3-oxazol-4-yl]-7-cyclopropyl-2H,3H,7H-pyrrolo[2,3-d]pyrimidin-2-one. Compound 225

3-[5-(4-Chloro-3-fluorophenyl)-1,3-oxazol-4-yl]-7-cyclopropyl-2H,3H,7H-pyrrolo[2,3-d]pyrimidin-2-one (0.20 g, 0.534 mmol, 1.0 eq), 2-bromo-4,6-dimethylpyrimidine (0.150 g, 0.801 mmol, 1.5 eq), bromo(1,10-phenanthroline)(triphenylphosphine)copper(I) (0.031 g, 0.053 mmol, 0.1 eq), and tetrakis(triphenylphosphine)palladium (0.062 g, 0.053 mmol, 0.1 eq) were dissolved in anhydrous dioxane (2.0 mL) and sparged with argon for 10 minutes. Then, cesium carbonate (0.348 g, 1.07 mmol, 2.0 eq) was added, and the reaction mixture was stirred for 10 hours at 100° C. The reaction mixture was cooled to room temperature and diluted with dichloromethane. Next, brine was added, and the reaction mixture was extracted with dichloromethane 3 times. The organic layers were collected and combined, dried over sodium sulfate, and concentrated under reduced pressure to give a crude material. This crude material was dissolved in DMSO and purified by reverse phase column chromatography (C18-SiO₂, 0-50% MeCN in water) to give 3-[5-(4-chloro-3-fluorophenyl)-2-(4,6-dimethylpyrimidin-2-yl)-1,3-oxazol-4-yl]-7-cyclopropyl-2H,3H,7H-pyrrolo[2,3-d]pyrimidin-2-one (0.017 g, 0.53 mmol, 6%).

LCMS: 2.743, [M+H]⁺=477.10, 96% @ 220 nm

¹H NMR (400 MHz, DMSO-d6) δ ppm 8.62 (s, 1H), 7.77 (t, J=8.2 Hz, 1H), 7.52-7.47 (m, 2H), 7.37 (dd, J=8.3, 1.9 Hz, 1H), 7.24 (d, J=4.0 Hz, 1H), 6.33 (d, J=4.0 Hz, 1H), 3.41-3.37 (m, 1H), 2.56 (s, 6H), 1.01 (d, J=5.5 Hz, 4H).

Compounds Prepared in an Analogous Manner to Compound 225:

| | Exact | LCMS | |
|---|---|---|---|
| Compound | Mass | Found | Assignment |
| 226 | 444.1 | 444.9 | (M + H)⁺ |
| 227 | 450.1 | 451.1 | (M + H)⁺ |
| 228 | 455.1 | 456.1 | (M + H)⁺ |
| 229 | 470.11 | 470.9 | (M + H)⁺ |
| 230 | 481.12 | 481.9 | (M + H)⁺ |
| 231 | 484.13 | 485.0 | (M + H)⁺ |

### Synthesis of Compound 232.

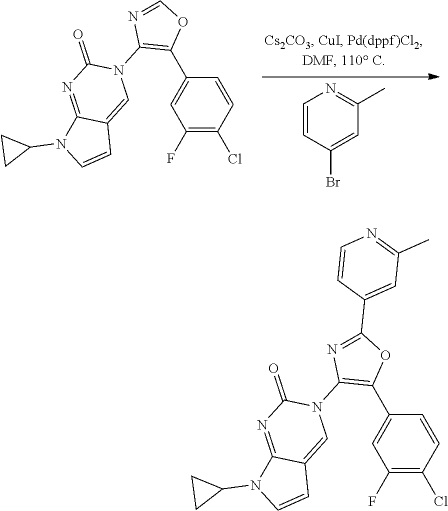

### 3-[5-(4-chloro-3-fluorophenyl)-2-(2-methylpyridin-4-yl)-1,3-oxazol-4-yl]-7-cyclopropyl-2H,3H,7H-pyrrolo[2,3-d]pyrimidin-2-one. Compound 232

4-Bromo-2-methylpyridine (0.023 g, 0.135 mmol, 1.0 eq), 3-[5-(4-chloro-3-fluorophenyl)-1,3-oxazol-4-yl]-5-cyclopropyl-2H,3H,5H-pyrrolo[3,2-d]pyrimidin-2-one (0.050 g, 0.135 mmol, 1.0 eq), and cesium carbonate (0.132 g, 0.405 mmol, 3.0 eq) were combined in a reaction vial and dissolved in anhydrous DMF (2.0 mL). The reaction mixture was then bubbled with argon for 10 minutes and copper(I) iodide (0.003 g, 0.013 mmol, 0.1 eq) and 1,1′-bis(diphenylphosphino)ferrocene dichloropalladium (II) (0.01 g, 0.013 mmol, 0.1 eq) were added. The reaction tube was sealed, and the mixture was stirred for 5 hours at 70° C. The reaction mixture was diluted with ethyl acetate and washed with brine. The layers were separated, and the aqueous layer was extracted 2 times with ethyl acetate. The combined organic layers were dried over sodium sulfate and concentrated under reduced pressure. The crude material was purified by silica gel column chromatography (0-5% MeOH in DCM) to give 3-[5-(4-chloro-3-fluorophenyl)-2-(2-methylpyridin-4-yl)-1,3-oxazol-4-yl]-7-cyclopropyl-2H,3H,7H-pyrrolo[2,3-d]pyrimidin-2-one (0.003 g, 0.006 mmol, 4%).

LCMS: 2.180, [M+H]⁺=462.0, 98% @ 220 nm

¹H NMR (400 MHz, DMSO-d6) δ ppm 8.70 (d, J=5.2 Hz, 1H), 8.61 (s, 1H), 8.03 (s, 1H), 7.90 (s, 1H), 7.82 (d, J=10.3 Hz, 1H), 7.74 (d, J=7.8 Hz, 1H), 7.33 (d, J=8.2 Hz, 1H), 7.24 (d, J=3.7 Hz, 1H), 6.33 (d, J=4.0 Hz, 1H), 2.62 (s, 3H), 1.01 (d, J=5.5 Hz, 4H).

Compounds Prepared in an Analogous Manner to Compound 232:

| | Exact | LCMS | |
|---|---|---|---|
| Compound | Mass | Found | Assignment |
| 233 | 462.10 | 463.1 | (M + H)⁺ |
| 234 | 463.13 | 463.7 | (M + H)⁺ |

### Synthesis of Compound 235.

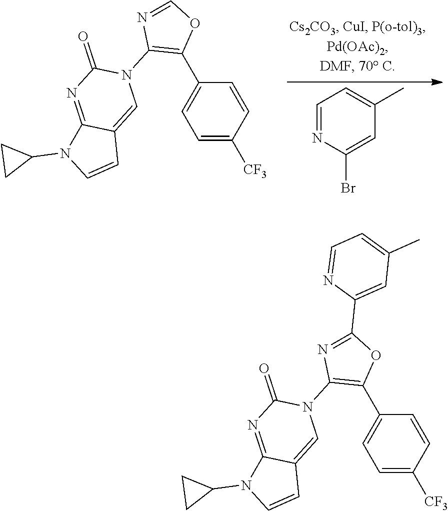

5-Cyclopropyl-3-{5-[4-(trifluoromethyl)phenyl]-1,3-oxazol-4-yl}-2H,3H,5H-pyrrolo[3,2-d]pyrimidin-2-one was synthesized in a similar fashion to 7-cyclopropyl-3-[5-(4-fluorophenyl)-1,3-oxazol-4-yl]-2H,3H,7H-pyrrolo[2,3-d]pyrimidin-2-one as shown above.

### 7-cyclopropyl-3-[2-(4-methylpyridin-2-yl)-5-[4-(trifluoromethyl)phenyl]-1,3-oxazol-4-yl]-2H,3H,7H-pyrrolo[2,3-d]pyrimidin-2-one. Compound 235

5-Cyclopropyl-3-{5-[4-(trifluoromethyl)phenyl]-1,3-oxazol-4-yl}-2H,3H,5H-pyrrolo[3,2-d]pyrimidin-2-one (0.10 g, 0.254 mmol, 1.0 eq), 2-bromo-4-methylpyridine (0.087 g, 0.507 mmol, 2.0 eq), and cesium carbonate (0.248 g, 0.761 mmol, 3.0 eq) were combined in a reaction vial and dissolved in anhydrous DMF (4.0 mL). The reaction mixture was sparged with argon for 10 minutes and tri-o-tolylphosphine (0.015 g, 0.051 mmol, 0.2 eq) and palladium(II) acetate (0.006 g, 0.025 mmol, 0.1 eq) were added. The reaction tube was sealed, and the mixture was stirred for 18 hours at 70° C. The reaction mixture was allowed to cool to room temperature and filtered through a pad of celite eluting with dichloromethane in methanol (1:1). The filtrate was concentrated under reduced pressure to give the crude product. The crude material was purified by silica gel column chromatography (0-5% MeOH in DCM) to give 7-cyclopropyl-3-[2-(4-methylpyridin-2-yl)-5-[4-(trifluoromethyl)phenyl]-1,3-oxazol-4-yl]-2H,3H,7H-pyrrolo[2,3-d]pyrimidin-2-one (0.007 g, 0.015 mmol, 6%).

LCMS: 2.103, [M+H]⁺=478.19, 97.4% @ 220 nm

¹H NMR (300 MHz, DMSO-d₆) δ ppm 8.65 (s, 1H), 8.54 (s, 1H), 7.44 (q, J=6.7 Hz, 6H), 7.21 (d, J=4.0 Hz, 1H), 6.28 (d, J=4.1 Hz, 1H), 1.00 (d, J=5.6 Hz, 4H).

Compounds Synthesized in an Analogous Manner to Compound 235:

| | Exact | LCMS | |
|---|---|---|---|
| Compound | Mass | Found | Assignment |
| 236 | 513.14 | 514.2 | (M + H)⁺ |
| 237 | 527.16 | 528.2 | (M + H)⁺ |

### Synthesis of Compound 238.

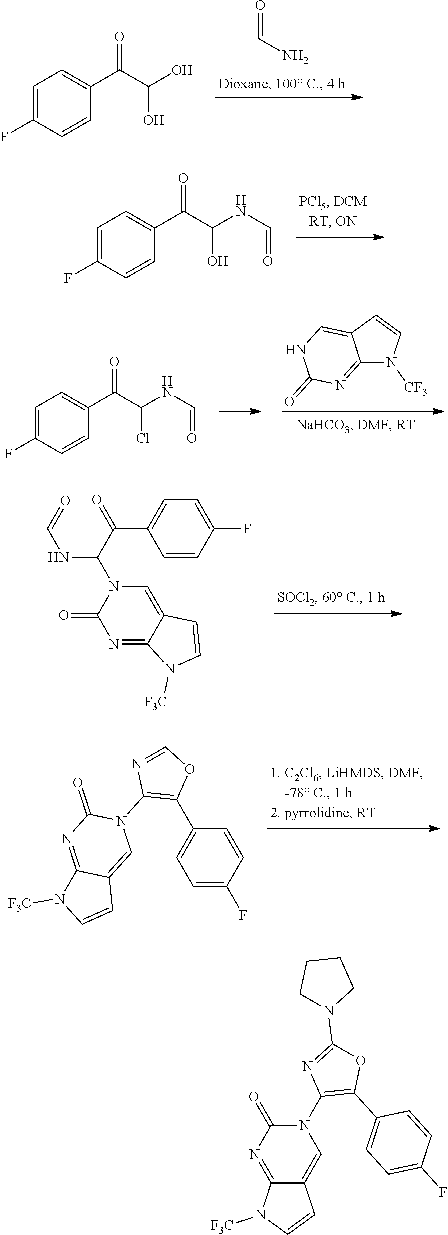

### N-[2-(4-fluorophenyl)-1-hydroxy-2-oxoethyl]formamide

4-Fluorophenylglyoxal hydrate (10.0 g, 58.8 mmol, 1.0 eq) and formamide (9.37 mL, 235 mmol, 4.0 eq) were dissolved in dioxane (150 mL), and the reaction mixture was stirred for 4 hours at 100° C. Upon cooling to room temperature, the dioxane was evaporated, and the obtained crude material was triturated with hexane/ethyl acetate (8:2 v/v). The resulting precipitate was collected by vacuum filtration, washed with diethyl ether, and dried under vacuum to give N-[2-(4-fluorophenyl)-1-hydroxy-2-oxoethyl]formamide (8.5 g, 43.1 mmol, 73%) as a white solid.

¹H NMR (400 MHz, DMSO-d6) δ ppm 8.97 (d, J=8.7 Hz, 1H), 8.17-8.00 (m, 3H), 7.40-7.35 (m, 2H), 6.78 (d, J=7.4 Hz, 1H), 6.40-6.32 (m, 1H).

### N-[1-chloro-2-(4-fluorophenyl)-2-oxoethyl]formamide

N-[2-(4-Fluorophenyl)-1-hydroxy-2-oxoethyl]formamide (8.5 g, 43.1 mmol, 1.0 eq) was dissolved in dichloromethane (170 mL). Phosphorus pentachloride (6.28 g, 30.2 mmol, 0.7 eq) was added, and the resulting mixture was stirred at room temperature for 16 hours. The solvent was removed under reduced pressure. The obtained solid was triturated with a hexanes/ethyl acetate mixture (9:1, v/v). The resulting solid was collected by vacuum filtration, washed with diethyl ether, and dried under vacuum to give N-[1-chloro-2-(4-fluorophenyl)-2-oxoethyl]formamide (8.5 g, 37.5 mmol, 87%) as a white solid. The product was used in the next step without further purification.

¹H NMR (300 MHz, DMSO-d6) δ ppm 8.97 (d, J=8.6 Hz, 1H), 8.11-8.03 (m, 3H), 7.43-7.36 (m, 2H), 6.35 (dd, J=8.7, 0.8 Hz, 1H).

### N-[2-(4-fluorophenyl)-2-oxo-1-[2-oxo-7-(trifluoromethyl)-2H,3H,7H-pyrrolo[2,3-d]pyrimidin-3-yl]ethyl]formamide

7-(Trifluoromethyl)-2H,3H,7H-pyrrolo[2,3-d]pyrimidin-2-one (2.6 g, 12.4 mmol, 0.805 eq) was added to a solution of sodium bicarbonate (6.48 g, 77.1 mmol, 5.0 eq) in DMF (70.0 mL), and the reaction mixture was stirred for 30 minutes at 0° C. Then, N-[1-chloro-2-(4-fluorophenyl)-2-oxoethyl]formamide (3.5 g, 15.4 mmol, 1.0 eq) was added, and the reaction was carried out at room temperature for 16 hours. The reaction mixture was diluted with ethyl acetate and washed with brine three times. The organic layer was dried over sodium sulfate and evaporated. The crude material was purified by silica gel column chromatography (0-3% MeOH in DCM) to afford N-[2-(4-fluorophenyl)-2-oxo-1-[2-oxo-7-(trifluoromethyl)-2H,3H,7H-pyrrolo[2,3-d]pyrimidin-3-yl]ethyl]formamide (2.88 g, 6.93 mmol, 45%) as a white solid.

UPLC-MS: 2.93 min, [M+H]⁺=383.50, 92%

### 3-[5-(4-fluorophenyl)-1,3-oxazol-4-yl]-7-(trifluoromethyl)-2H,3H,7H-pyrrolo[2,3-d]pyrimidin-2-one

N-[2-(4-Fluorophenyl)-2-oxo-1-[2-oxo-7-(trifluoromethyl)-2H,3H,7H-pyrrolo[2,3-d]pyrimidin-3-yl]ethyl]formamide (2.88 g, 6.93 mmol, 1.0 eq) was dissolved in thionyl chloride (18.8 mL, 261 mmol, 40.0 eq) and the mixture was heated to 60° C. for 1 hour. The reaction was quenched with methanol, and the volatiles were removed under reduced pressure. The obtained crude material was suspended in diethyl ether and a precipitate formed. The solid was collected by vacuum filtration and dried under vacuum to give 3-[5-(4-fluorophenyl)-1,3-oxazol-4-yl]-7-(trifluoromethyl)-2H,3H,7H-pyrrolo[2,3-d]pyrimidin-2-one (2.09 g, 5.28 mmol, 81%) as a yellow solid.

UPLC-MS: 3.23 min, [M+H]⁺=364.45, 92%

¹H NMR (400 MHz, DMSO-d6) δ ppm 8.79 (s, 1H), 8.68 (s, 1H), 7.61 (d, J=4.3 Hz, 1H), 7.57-7.45 (m, 2H), 7.33 (t, J=8.9 Hz, 2H), 6.63 (d, J=4.3 Hz, 1H).

### 3-[5-(4-fluorophenyl)-2-(pyrrolidin-1-yl)-1,3-oxazol-4-yl]-7-(trifluoromethyl)-2H,3H,7H-pyrrolo[2,3-d]pyrimidin-2-one. Compound 238

3-[5-(4-Fluorophenyl)-1,3-oxazol-4-yl]-7-(trifluoromethyl)-2H,3H,7H-pyrrolo[2,3-d]pyrimidin-2-one (0.050 g, 0.122 mmol, 1.0 eq) and hexachloroethane (0.058 g, 0.244 mmol, 2.0 eq) were dissolved in a mixture of anhydrous DMF (1.0 mL) and anhydrous THF (2.5 mL). Then, pyrrolidine (0.050 mL, 0.611 mmol, 5.0 eq) was added. The resulting solution was cooled to −78° C., and lithium bis(trimethylsilyl)amide solution (1.0 M in THF, 0.244 mL, 0.244 mmol, 2.0 eq) was added dropwise. The reaction mixture was allowed to warm to room temperature and was stirred for 16 hours. The reaction was quenched with saturated aqueous ammonium chloride, and the aqueous layer was extracted with dichloromethane. The combined organic layers were washed with brine, dried over magnesium sulfate, and concentrated under reduced pressure. The crude material was purified by preparative HPLC (C18 column, 10-100% MeCN in water with 0.1% TFA) to afford 3-[5-(4-fluorophenyl)-2-(pyrrolidin-1-yl)-1,3-oxazol-4-yl]-7-(trifluoromethyl)-2H,3H,7H-pyrrolo[2,3-d]pyrimidin-2-one (0.019 g, 0.043 mmol, 35%) as a white solid.

LCMS: 6.53, [M+H]⁺=438.80, 98% @ 220 nm

¹H NMR (300 MHz, DMSO-d6) δ ppm 8.74 (s, 1H), 7.59 (d, J=4.3 Hz, 1H), 7.33-7.21 (m, 4H), 6.61 (d, J=4.3 Hz, 1H), 3.55-3.49 (m, 4H), 2.01-1.95 (m, 4H).

Compounds Prepared in an Analogous Manner to Compound 238:

| | Exact | LCMS | |
|---|---|---|---|
| Compound | Mass | Found | Assignment |
| 239 | 437.33 | 437.9 | (M + H)⁺ |
| 240 | 444.35 | 444.9 | (M + H)⁺ |
| 241 | 444.35 | 444.9 | (M + H)⁺ |
| 242 | 481.41 | 481.6 | (M + H)⁺ |

### Synthesis of Compound 243.

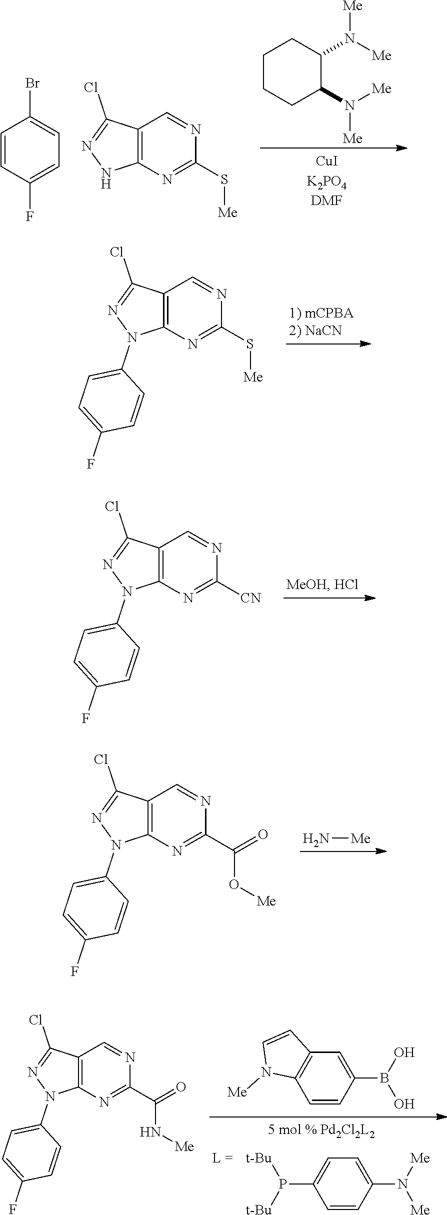

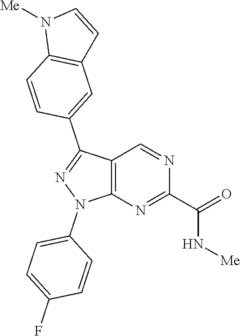 [SMILES: CNC(=O)c1ncc2c(-c3ccc4c(ccn4C)c3)nn(-c3ccc(F)cc3)c2n1]

### 3-chloro-1-(4-fluorophenyl)-6-(methylthio)-1H-pyrazolo[3,4-d]pyrimidine

3-Chloro-6-(methylsulfanyl)-1H-pyrazolo[3,4-d]pyrimidine (4.0 g, 19.9 mmol), 1-bromo-4-fluorobenzene (10.4 g, 59.6 mmol), tripotassium phosphate (12.6 g, 59.6 mmol), and copper iodide (3.78 g, 19.9 mmol) were dissolved in DMF (40 mL). The reaction was degassed with argon and (1R,2R)-1-N,2-N-dimethylcyclohexane-1,2-diamine (5.66 g, 39.8 mmol) was added. The reaction vial was sealed at stirred at 100° C. for 16 hours. The crude mixture was dry loaded onto silica and purified by silica gel column chromatography (24 g, 25% EtOAc in heptane) providing 3-chloro-1-(4-fluorophenyl)-6-(methylthio)-1H-pyrazolo[3,4-d]pyrimidine (3.33 g, 56.8%) as a white solid.

LCMS (ESI+): m/z 295.1 [M+H]⁺

### 3-chloro-1-(4-fluorophenyl)-1H-pyrazolo[3,4-d]pyrimidine-6-carbonitrile

3-Chloro-1-(4-fluorophenyl)-6-(methylsulfanyl)-1H-pyrazolo[3,4-d]pyrimidine (3.3 g, 11.1 mmol) was dissolved in dioxane (30 mL). 3-Chlorobenzene-1-carboperoxoic acid (4.08 g, 16.6 mmol) was added and the reaction solution was stirred at room temperature for 1 hour. The reaction mixture was tested with peroxide paper to confirm no more peroxide remained and then was concentrated under reduced pressure. The resulting sulfone, sulfoxide mixture was then dissolved in DMSO (40 mL). Sodium cyanide (1.35 g, 27.7 mmol) was added to the solution and the reaction was stirred for 10 minutes. The crude mixture was diluted with saturated aqueous sodium bicarbonate and the product was extracted with ethyl acetate. The crude product was dry loaded onto silica gel and purified by column chromatography (silica gel, 40 g, 20% EtOAc in heptane) providing 3-chloro-1-(4-fluorophenyl)-1H-pyrazolo[3,4-d]pyrimidine-6-carbonitrile (2.30 g, 75.9%) as a pale yellow solid.

LCMS (ESI+): m/z 274.1 [M+H]⁺

### methyl 3-chloro-1-(4-fluorophenyl)-1H-pyrazolo[3,4-d]pyrimidine-6-carboxylate

3-Chloro-1-(4-fluorophenyl)-1H-pyrazolo[3,4-d]pyrimidine-6-carbonitrile (2.2 g, 8.03 mmol) was dissolved in hydrochloric acid in methanol (6.16 g, 90.0 mmol) and stirred for 16 hours at 60° C. The reaction mixture was then concentrated under reduced pressure. The crude product was dry loaded onto silica gel and purified by column chromatography (silica gel, 24 g, 50% EtOAc in heptane) providing methyl 3-chloro-1-(4-fluorophenyl)-1H-pyrazolo[3,4-d]pyrimidine-6-carboxylate (1.97 g, 80.0%) as a white solid.

LCMS (ESI+): m/z 307.0 [M+H]⁺

### 3-chloro-1-(4-fluorophenyl)-N-methyl-1H-pyrazolo[3,4-d]pyrimidine-6-carboxamide

Methyl 3-chloro-1-(4-fluorophenyl)-1H-pyrazolo[3,4-d]pyrimidine-6-carboxylate (1.93 g, 6.42 mmol) was dissolved in methylamine in EtOH (1.59 g, 51.3 mmol) and stirred for 4 hours at 70° C. The resulting solution was concentrated under reduced pressure to provide 3-chloro-1-(4-fluorophenyl)-N-methyl-1H-pyrazolo[3,4-d]pyrimidine-6-carboxamide (1.85 g, 94.3%) as a white solid.

LCMS (ESI+): m/z 306.0 [M+H]⁺

### 1-(4-fluorophenyl)-N-methyl-3-(1-methyl-1H-indol-5-yl)-1H-pyrazolo[3,4-d]pyrimidine-6-carboxamide. Compound 243

3-Chloro-1-(4-fluorophenyl)-N-methyl-1H-pyrazolo[3,4-d]pyrimidine-6-carboxamide (0.050 g, 163 μmol), (1-methyl-1H-indol-5-yl)boronic acid (57.0 mg, 326 μmol), bis(4-(di-tert-butylphosphanyl)-N,N-dimethylaniline); dichloropalladium (5.77 mg, 8.15 μmol), and potassium acetate (47.9 mg, 489 μmol) were combined, and the reaction vial was degassed with argon. Degassed butan-1-ol (1 mL) was added to the reaction vial and it was stirred at 70° C. for 16 hours. The reaction was dry loaded onto silica gel and purified by column chromatography (silica gel, 40 g, 90% EtOAc in heptane) to provide 1-(4-fluorophenyl)-N-methyl-3-(1-methyl-1H-indol-5-yl)-1H-pyrazolo[3,4-d]pyrimidine-6-carboxamide (60.0 mg, 92.0%) as a pale yellow solid.

LCMS (ESI+): m/z 401.1 [M+H]⁺

1H NMR (400 MHz, DMSO-d6) d ppm 9.88 (bs, 1H), 9.00 (bs, 1H), 8.40 (s, 1H), 8.45 (s, 2H), 8.02 (d, J=7.82 Hz, 1H), 7.65 (d, J=8.56 Hz, 1H), 7.42-7.57 (m, 3H), 6.62 (bs, 1H), 3.88 (bs, 3H), 2.86-2.97 (m, 3H).

Additional Compounds Prepared in an Analogous Manner to Compound 243:

| | Exact | LCMS | |
|---|---|---|---|
| Compound | Mass | Found | Assignment |
| 244 | 381.0793 | 382.1 | (M + H)⁺ |
| 245 | 375.1495 | 376.3 | (M + H)⁺ |
| 246 | 347.1182 | 348.2 | (M + H)⁺ |

### Synthesis of Compound 247.

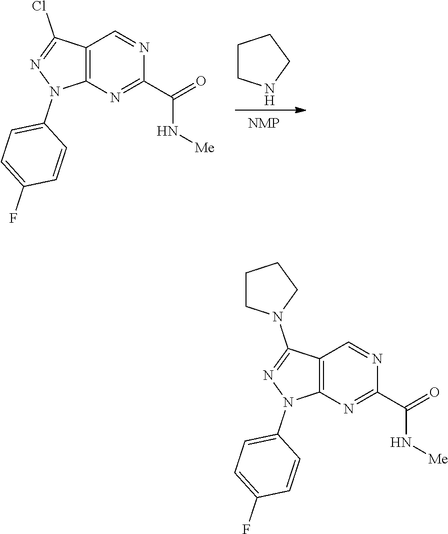

### 1-(4-fluorophenyl)-N-methyl-3-(pyrrolidin-1-yl)-1H-pyrazolo[3,4-d]pyrimidine-6-carboxamide

A solution of 3-chloro-1-(4-fluorophenyl)-N-methyl-1H-pyrazolo[3,4-d]pyrimidine-6-carboxamide (0.040 g, 130 μmol) and pyrrolidine (91.7 mg, 1.29 mmol) in 1-methylpyrrolidin-2-one (0.5 mL) was heated to 180° C. in the microwave for 1 hour. The reaction was cooled and the mixture quenched with water (5 mL). A yellow precipitate formed. It was collected by vacuum filtration and dried under vacuum to afford 1-(4-fluorophenyl)-N-methyl-3-(pyrrolidin-1-yl)-1H-pyrazolo[3,4-d]pyrimidine-6-carboxamide (40 mg, 90% yield). LCMS (ESI+): m/z 341.1 [M+H]⁺

### Synthesis of Compounds 37 & 22

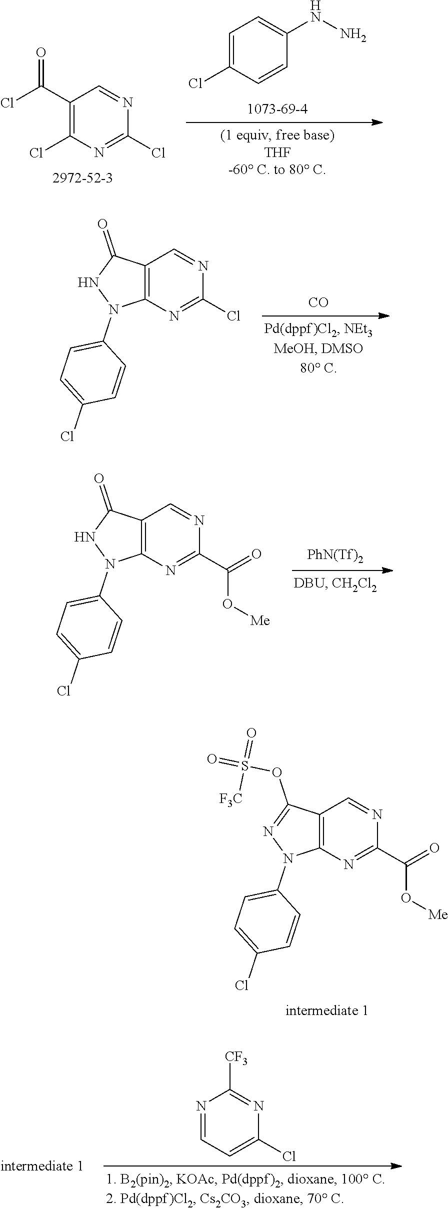

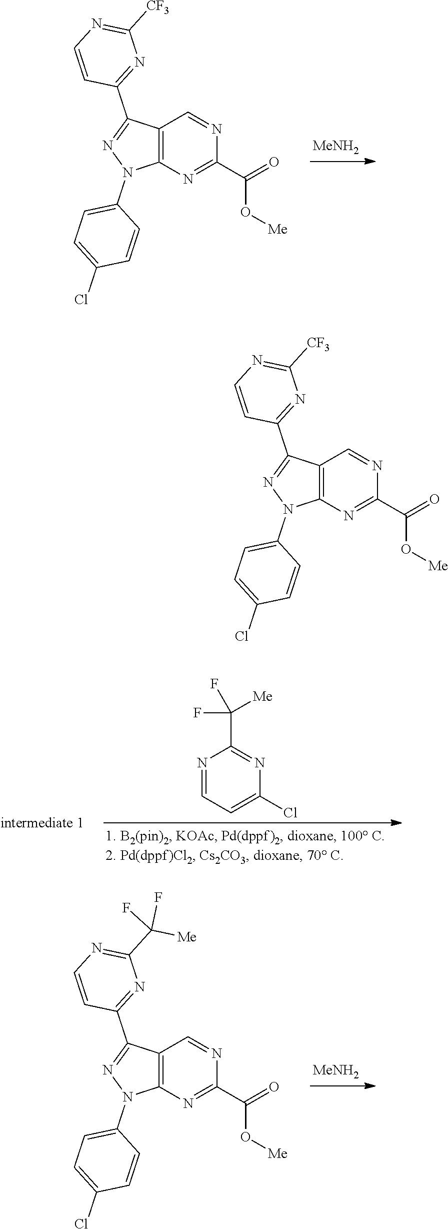 [SMILES: "MeNH2" ; COC(=O)c1ncc2c(-c3ccnc(C(F)(F)F)n3)nn(-c3ccc(Cl)cc3)c2n1 ; COC(=O)c1ncc2c(-c3ccnc(C(F)(F)F)n3)nn(-c3ccc(Cl)cc3)c2n1 ; "MeNH2" ; CC(F)(F)c1nccc(Cl)n1 ; COC(=O)c1ncc2c(-c3ccnc(C(C)(F)F)n3)nn(-c3ccc(Cl)cc3)c2n1]

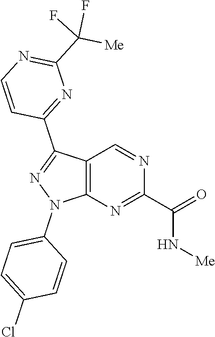 [SMILES: CNC(=O)c1ncc2c(-c3ccnc(C(C)(F)F)n3)nn(-c3ccc(Cl)cc3)c2n1]

### 6-chloro-1-(4-chlorophenyl)-1,2-dihydro-3H-pyrazolo[3,4-d]pyrimidin-3-one

To a solution of (4-chlorophenyl)hydrazine (87.7 g, 615 mmol) in THF (2.0 L) was added a solution of 2,4-dichloro-5-pyrimidinecarbonyl chloride (130 g, 615 mmol) in THF (400 mL) at -60° C. After stirring at −60° C. for 2 hours, the mixture was heated to 80° C. and stirred for 16 hours. This process was repeated eight additional times and the 9 runs were combined. After cooling to 20° C., the crude mixture was concentrated under reduced pressure. The residue was triturated with MeOH (4.0 L), and the resulting precipitate was collected by vacuum filtration and dried under high vacuum to obtain 6-chloro-1-(4-chlorophenyl)-1,2-dihydro-3H-pyrazolo[3,4-d]pyrimidin-3-one (1050 g, 70.3% yield) as a yellow solid.

¹H NMR: 400 MHz, DMSO d ppm 7.62-7.64 (m, 2H), 8.06-8.08 (d, 2H, J=8.8 Hz), 9.18 (s, 1H), 12.59 (s, 1H)

### methyl 1-(4-chlorophenyl)-3-oxo-2,3-dihydro-1H-pyrazolo[3,4-d]pyrimidine-6-carboxylate

To a solution of 6-chloro-1-(4-chlorophenyl)-1,2-dihydro-3H-pyrazolo[3,4-d]pyrimidin-3-one (100 g, 332 mmol, 1 eq) in MeOH (1466 mL) and DMSO (366 mL) were added Pd(dppf)Cl₂·CH₂Cl₂ (13.3 g, 16.6 mmol, 0.05 eq) and TEA (101 g, 0.99 mol, 138 mL, 3 eq). The mixture was stirred at 80° C. for 12 hours under CO (2 MPa). An additional nine reactions were set up as described above. After cooling to 20° C., all ten reaction mixtures were combined and concentrated under reduced pressure. The residue was triturated with MeOH (6.0 L), and the resulting solid was collected by vacuum filtration and dried under high vacuum to give methyl 1-(4-chlorophenyl)-3-oxo-2,3-dihydro-1H-pyrazolo[3,4-d]pyrimidine-6-carboxylate (940 g, 82% yield) as a yellow solid.

¹H NMR: 400 MHz, DMSO-d6 d ppm 3.95 (s, 3H), 7.61-7.67 (m, 2H), 8.18 (d, J=8.99 Hz, 2H), 9.40 (s, 1H), 12.53-12.72 (m, 1H)

### methyl 1-(4-chlorophenyl)-3-(((trifluoromethyl)sulfonyl)oxy)-1H-pyrazolo[3,4-d]pyrimidine-6-carboxylate

To a suspension of methyl 1-(4-chlorophenyl)-3-oxo-2,3-dihydro-1H-pyrazolo[3,4-d]pyrimidine-6-carboxylate (150 g, 492 mmol) in DCM (1.50 L) was added DBU (112 g, 738 mmol). The suspension became clear, and 1,1,1-trifluoro-N-phenyl-N-(trifluoromethylsulfonyl)methanesulfonamide (193 g, 542 mmol) was added portion-wise at 20° C. The mixture was stirred at 20° C. for 2 hours. Five additional reactions were set up as detailed above. All six reaction mixtures were combined. The combined mixture was treated with water (5.0 L) and extracted with dichloromethane (3×3.0 L). The organic layer was dried over Na₂SO₄, filtered, and concentrated under reduced pressure. The residue was purified by column chromatography on silica gel (5-35% EtOAc in petroleum ether) to obtain methyl 1-(4-chlorophenyl)-3-(((trifluoromethyl)sulfonyl)oxy)-1H-pyrazolo[3,4-d]pyrimidine-6-carboxylate (560 g, 41.6% yield) as a white solid.

¹H NMR: 400 MHz, DMSO d ppm 3.98 (s, 3H), 7.76 (d, 2H, J=9.0 Hz), 8.11 (d, 2H, J=8.9 Hz), 9.80 (s, 1H)

### methyl 1-(4-chlorophenyl)-3-(2-(trifluoromethyl)pyrimidin-4-yl)-1H-pyrazolo[3,4-cl]pyrimidine-6-carboxylate

A mixture of methyl 1-(4-chlorophenyl)-3-(trifluoromethylsulfonyloxy)pyrazolo[3,4-d] pyrimidine-6-carboxylate (0.23 mmol, 0.10 g), bis(pinacolato)diboron (0.34 mmol, 0.087 g), [1, 1′-bis(diphenylphosphino)ferrocene]dichloropalladium(II) (0.0458 mmol, 0.0335 g), and potassium acetate (0.503 mmol, 0.0494 g) in dioxane (29 mmol, 2.6 g, 2.5 mL) was stirred at 110° C. for 3 hours. Then, 4-chloro-2-(trifluoromethyl)pyrimidine (0.34 mmol, 0.063 g, 0.041 mL) and cesium carbonate (0.687 mmol, 0.224 g) were added and the mixture was stirred at 110° C. for 15 minutes. After cooling to ambient temperature, the mixture was filtered through a short column of silica gel eluting with DCM. The eluting solution was then concentrated in vacuo. The residue was dissolved in DCM and an insoluble material was filtered off. The filtrate was concentrated in vacuo. The residue was suspended in isopropanol, and the resulting precipitate was collected by vacuum filtration to afford methyl 1-(4-chlorophenyl)-3-(2-(trifluoromethyl)pyrimidin-4-yl)-1H-pyrazolo[3,4-d]pyrimidine-6-carboxylate (0.12 mmol, 0.050 g, 50% yield) as a gray solid.

¹H-NMR: CDCl₃ d ppm 10.27 (1H, s), 9.10 (1H, d, J=5.5 Hz), 8.44 (1H, d, J=5.5 Hz), 8.38 (2H, d, J=8.6 Hz), 7.61 (2H, d, J=9.2 Hz), 4.14 (3H, s).

LCMS (ESI+): m/z 435 [M+H]⁺

### 1-(4-chlorophenyl)-N-methyl-3-(2-(trifluoromethyl)pyrimidin-4-yl)-1H-pyrazolo[3,4-d]pyrimidine-6-carboxamide. Compound 37

A mixture of methyl 1-(4-chlorophenyl)-3-(2-(trifluoromethyl)pyrimidin-4-yl)pyrazolo[3,4-d]pyrimidine-6-carboxylate (10 mg, 0.023 mmol) and methylamine solution (33 wt % in

Ethanol, 10 equiv, 0.23 mmol, 0.029 mL) in DMSO (1 mL) was stirred at 50° C. for 1.5 hours. Then, an additional amount of methylamine solution (0.5 mL) was added and the mixture was stirred at 80° C. in a sealed tube for 45 minutes. After cooling to ambient temperature, the mixture was poured into water and stirred for 10 minutes. A precipitate formed and was collected by vacuum filtration, washed with water, and washed with a minimal amount of methanol. The solid was then dried under vacuum to afford 1-(4-chlorophenyl)-N-methyl-3-(2-(trifluoromethyl)pyrimidin-4-yl)pyrazolo[3,4-d]pyrimidine-6-carboxamide (5 mg, 0.012 mmol, 50% Yield) as a pale yellow solid.

¹H-NMR: DMSO-D₆ d ppm 9.93 (1H, s), 9.28 (1H, d, J=4.9 Hz), 9.12 (1H, d, J=4.9 Hz), 8.62 (1H, d, J=5.5 Hz), 8.43-8.42 (2H, m), 7.77 (2H, d, J=9.2 Hz), 2.88 (3H, d, J=4.9 Hz).

LCMS (ESI+): m/z 434 [M+H]⁺

### Methyl 1-(4-chlorophenyl)-3-(2-(1,1-difluoroethyl)pyrimidin-4-yl)-1H-pyrazolo[3,4-d]pyrimidine-6-carboxylate

A mixture of methyl 1-(4-chlorophenyl)-3-(trifluoromethylsulfonyloxy)pyrazolo[3,4-d]pyrimidine-6-carboxylate (0.65 g, 1.5 mmol), bis(pinacolato)diboron (1.9 mmol, 0.49 g), [1,1′-bis(diphenylphosphino)ferrocene] dichloropalladium(II) (0.30 mmol, 0.22 g), and potassium acetate (3.3 mmol, 0.32 g) in dioxane (15 mL) was stirred at 100° C. for 1 hour. Then, 4-chloro-2-(1,1-difluoroethyl)pyrimidine (1.9 mmol, 0.35 g) and cesium carbonate (3.0 mmol, 0.97 g) were added and the mixture was stirred at 100° C. for 30 minutes. After cooling to ambient temperature, the mixture was filtered through a short column of silica gel eluting with DCM. The elution solution was then concentrated in vacuo. The residue was dissolved in DCM and an insoluble material was filtered off. The filtrate was concentrated in vacuo and the residue was purified by silica gel column chromatography (0-100% EtOAc in hexanes) to provide methyl 1-(4-chlorophenyl)-3-(2-(1,1-difluoroethyl) pyrimidin-4-yl)pyrazolo[3,4-d]pyrimidine-6-carboxylate (461 mg, 1.07 mmol, 72% yield) as a solid.

¹H NMR: 400 MHz, CDCl₃ d ppm 2.20 (t, 3H, J=18.45 Hz), 4.14 (s, 3H), 7.56-7.67 (m, 2H), 8.32 (d, 1H, J=5.13 Hz), 8.35-8.42 (m, 2H), 9.05 (d, 1H, J=5.1 Hz), 10.29 (s, 1H).

LCMS (ESI+): m/z 431.0 [M+H]⁺

### 1-(4-chlorophenyl)-3-(2-(1,1-difluoroethyl)pyrimidin-4-yl)-N-methyl-1H-pyrazolo[3,4-d]pyrimidine-6-carboxamide. Compound 22

Methyl 1-(4-chlorophenyl)-3-(2-(1,1-difluoroethyl)pyrimidin-4-yl)pyrazolo[3,4-d] pyrimidine-6-carboxylate (461 mg, 1.07 mmol) and methylamine (40% in methanol, 5 mL, 49 mmol) were added to THF (5 mL), and the mixture was stirred at room temperature for 60 minutes. The mixture was concentrated in vacuo, and the residue was purified by silica gel chromatography (0-20% EtOAc in DCM) to obtain product as a solid. This material was then suspended into 5 mL of i-PrOH, and the resulting precipitate was collected by vacuum filtration to provide 1-(4-chlorophenyl)-3-(2-(1,1-difluoroethyl)pyrimidin-4-yl)-N-methyl-1H-pyrazolo[3,4-d]pyrimidine-6-carboxamide (293 mg, 0.68 mmol, 64% yield) as a white solid.

¹H NMR 400 MHz, CDCl₃ d ppm 2.20 (t, J=18.51 Hz, 3H), 3.16 (d, J=5.13 Hz, 3H), 7.53-7.65 (m, 2H), 8.19 (d, J=4.00 Hz, 1H), 8.31 (d, J=5.13 Hz, 1H), 8.34-8.41 (m, 2H), 9.04 (d, J=5.13 Hz, 1H), 10.17 (s, 1H).

LCMS (ESI+): m/z 430.0 [M+H]⁺

LC/MS (The gradient was 5% B in 0.40 min and 5-95% B in 2.60 min, hold on 95% B in

1.00 min, and then 95-5% B in 0.01 min, the flow rate was 1.0 ml/min. Mobile phase A was 0.04% Trifluoroacetic Acid in water, mobile phase B was 0.02% Trifluoroacetic Acid in acetonitrile. The column used for chromatography was a Kinetex C18 2.1*50 mm, 5 um. Detection methods are diode array (DAD), and evaporative light scattering detection (ELSD). MS mode was positive electrospray ionization. MS range was 100-1000.

| | Exact | LCMS | |
|---|---|---|---|
| Compound | Mass | Found | Assignment |
| 37 | 433.0666 | 434.0 | [M + H]⁺ |
| 22 | 429.0916 | 430.0 | [M + H]⁺ |

### Synthesis of Compound 8.

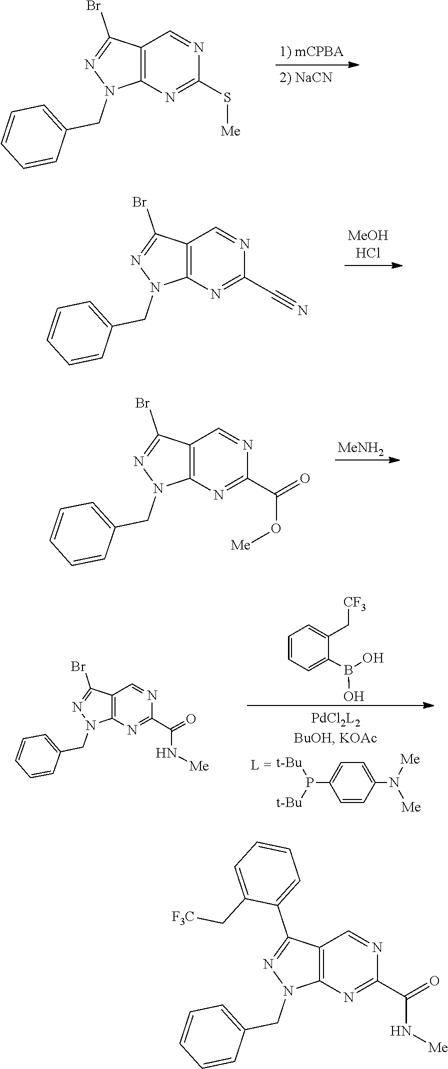

### 1-benzyl-3-bromo-1H-pyrazolo[3,4-d]pyrimidine-6-carbonitrile

To a stirring solution of 1-benzyl-3-bromo-6-(methylthio)-1H-pyrazolo[3,4-d]pyrimidine (5.4 g, 16.1 mmol) in THF (10 mL) at 0° C., 3-chlorobenzene-1-carboperoxoic acid (5.3 g, 1.5 equiv) was slowly added, and the mixture was allowed to stir for 30 minutes at room temperature. The reaction mixture was checked with iodide paper to confirm the consumption of peroxide. The solvent was removed in vacuo, and the solid was redissolved in DCM and mixed with aqueous NaHCO₃. The organic layer was separated, washed with brine, dried over MgSO₄, and concentrated in vacuo. The resulting solid was redissolved in DMSO (10 mL). To this stirring solution, sodium cyanide (1.97 g, 2.5 equiv) was added, and the mixture was allowed to stir for 30 minutes at room temperature. The reaction was diluted with water and EtOAc. The organic layer was separated, washed with brine, dried over MgSO₄, and concentrated in vacuo. The resulting solid was dry loaded onto silica gel and purified by column chromatography (silica gel, 80 g, 50% EtOAc in heptane) to give 1-benzyl-3-bromo-1H-pyrazolo[3,4-d]pyrimidine-6-carbonitrile (3.0 g, 59%).

LCMS (ESI+): m/z 314.0 [M+H]⁺

### methyl 1-benzyl-3-bromo-1H-pyrazolo[3,4-d]pyrimidine-6-carboxylate

1-Benzyl-3-bromo-1H-pyrazolo[3,4-d]pyrimidine-6-carbonitrile (0.5 g, 1.59 mmol) was dissolved in hydrochloric acid in MeOH (3.0 M, 15 mL) and stirred for 16 hours at 60° C. The reaction mixture was cooled to room temperature and concentrated in vacuo to give methyl 1-benzyl-3-bromo-1H-pyrazolo[3,4-d]pyrimidine-6-carboxylate (365 mg, 66.1%) as a yellow solid.

LCMS (ESI+): m/z 347.0 [M+H]⁺

### 1-benzyl-3-bromo-N-methyl-1H-pyrazolo[3,4-d]pyrimidine-6-carboxamide

Methyl 1-benzyl-3-bromo-1H-pyrazolo[3,4-d]pyrimidine-6-carboxylate (365 mg, 1.05 mmol) was dissolved in methylamine (33% in EtOH, 2 mL) and heated to 60° C. for 16 hours. The reaction was concentrated in vacuo to give 1-benzyl-3-bromo-N-methyl-1H-pyrazolo[3,4-d]pyrimidine-6-carboxamide (361 mg, 99.4%) as a pale yellow solid.

LCMS (ESI+): m/z 346.0 [M+H]⁺

### 1-benzyl-N-methyl-3-(2-(2,2,2-trifluoroethyl)phenyl)-1H-pyrazolo[3,4-d]pyrimidine-6-carboxamide. Compound 8

1-Benzyl-3-bromo-N-methyl-1H-pyrazolo[3,4-d]pyrimidine-6-carboxamide (50 mg, 144 μmol), [2-(2,2,2-trifluoroethyl)phenyl]boronic acid (58.7 mg, 288 μmol), 4-{di-tert-butyl[dichloro({di-tert-butyl[4-(dimethylamino)phenyl]-λ⁵-phosphanyl})palladio]-λ⁵-phosphanyl}-N,N-dimethylaniline (5.11 mg, 7.20 μmol), and potassium acetate (42.3 mg, 432 μmol) were dissolved in butan-1-ol (1 mL) and water (0.1 mL). The reaction mixture was stirred for 16 hours at 80° C. Upon cooling, the reaction was concentrated onto silica gel and purified by column chromatography (silica gel, 24 g, 50% EtOAc in hexanes) to give 1-benzyl-N-methyl-3-(2-(2,2,2-trifluoroethyl)phenyl)-1H-pyrazolo[3,4-d]pyrimidine-6-carboxamide (38 mg, 62%) as a pale yellow solid.

LCMS (ESI+): m/z 426.2 [M+H]⁺

Additional Compounds Synthesized in an Analogous Manner to Compound 8:

| | Exact | LCMS | |
|---|---|---|---|
| Compound | Mass | Found | Assignment |
| 8 | 425.1463 | 426.2 | (M + H)⁺ |
| 15 | 411.1307 | 412.1 | (M + H)⁺ |
| 248 | 343.1433 | 344.1 | (M + H)⁺ |
| 249 | 361.1339 | 362.1 | (M + H)⁺ |
| 250 | 361.1339 | 362.1 | (M + H)⁺ |
| 251 | 333.159 | 334.1 | (M + H)⁺ |
| 252 | 379.1245 | 380.1 | (M + H)⁺ |
| 253 | 415.1557 | 416.1 | (M + H)⁺ |
| 254 | 423.1143 | 424.2 | (M + H)⁺ |
| 255 | 404.1397 | 405.5 | (M + H)⁺ |

Synthesis of Compound 256.

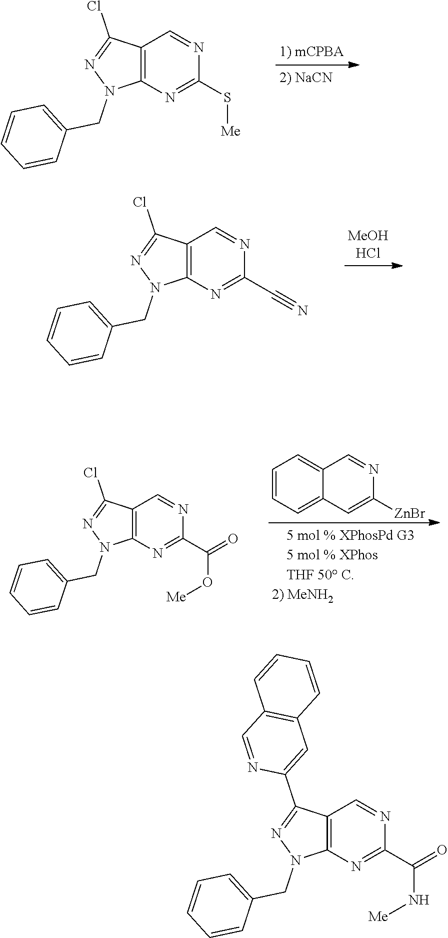

### methyl 1-benzyl-3-chloro-1H-pyrazolo[3,4-d]pyrimidine-6-carboxylate

1-Benzyl-3-chloro-6-(methylthio)-1H-pyrazolo[3,4-d]pyrimidine (198 mg, 1 Eq, 681 μmol) was dissolved in THF (2 mL). m-Chloro-perbenzoic acid (176 mg, 1.5 Eq, 1.02 mmol) was added, and the reaction solution was stirred at room temperature for 1 hour. The reaction mixture was tested with peroxide paper to confirm no more peroxide remained and then was concentrated under reduced pressure. This sulfone, sulfoxide mixture was then dissolved in DMSO (4 mL). Sodium cyanide (83.4 mg, 2.5 Eq, 1.70 mmol) was added to the solution and the reaction was stirred for 10 minutes at room temperature. The crude mixture was diluted with saturated aqueous sodium bicarbonate and the product was extracted with ethyl acetate. The organic solution was dried over sodium sulfate and concentrated. This crude material was then dissolved in HCl in methanol (3 M, 2 mL) and heated for 16 hours at 60° C. The mixture was cooled and concentrated under reduced pressure. The crude material was suspended in DCM and washed with saturated aqueous sodium bicarbonate. The organic solution was concentrated onto silica gel and purified by silica gel column chromatography (0-100% EtOAc in heptane) to yield methyl 1-benzyl-3-chloro-1H-pyrazolo[3,4-d]pyrimidine-6-carboxylate (110 mg, 363 μmol, 53.4%)

LCMS (ESI+): m/z 303.1 [M+H]⁺

### 1-benzyl-3-(isoquinolin-3-yl)-N-methyl-1H-pyrazolo[3,4-d]pyrimidine-6-carboxamide. Compound 256

Methyl 1-benzyl-3-chloro-1H-pyrazolo[3,4-d]pyrimidine-6-carboxylate (50.0 mg, 1 Eq, 165 μmol) dicyclohexyl(2′,4′,6′-triisopropyl-[1,1′-biphenyl]-2-yl)phosphane (3.94 mg, 0.05 Eq, 8.26 μmol), and Xphos Pd G3 (6.99 mg, 0.05 Eq, 8.26 μmol) were dissolved in minimal THF (0.75 mL) and isoquinolin-3-ylzinc(II) bromide (90.3 mg, 0.7 mL, 0.5 molar, 2 Eq, 330 μmol) was added. The reaction mixture was heated to 50° C. for 16 hours. Upon completion, the reaction was quenched with ammonium chloride and extracted with DCM. The organic layer was dried over sodium sulfate and concentrated to give the crude ester intermediate. This material was then dissolved in 33% methylamine in EtOH (5 mL) and the mixture was stirred at room temperature for 16 hours. The reaction mixture was then concentrated onto silica gel and purified by silica gel column chromatography (20% EtOAc [with 0.1% Et₃N] in heptanes) to provide 1-benzyl-3-(isoquinolin-3-yl)-N-methyl-1H-pyrazolo[3,4-d]pyrimidine-6-carboxamide (4.00 mg, 10.1 μmol, 6.14%) as a white solid.

LCMS: m/z=395.1, LCAP=99% @ 254 nm

¹H NMR: (DMSO-d6, 400 MHz) d ppm 10.29 (s, 1H), 9.21-9.20 (m, 1H), 8.59 (d, J=8.4 Hz, 1H), 8.35 (d, J=8.4 Hz, 2H), 8.12 (d, J=8.0 Hz, 1H), 7.93 (t, J=8.0 Hz, 1H), 7.74 (t, J=7.2 Hz, 1H), 7.44-7.37 (m, 5H), 5.95 (s, 2H), 2.97 (d, J=4.4 Hz, 3H).

### Synthesis of Compound 19.

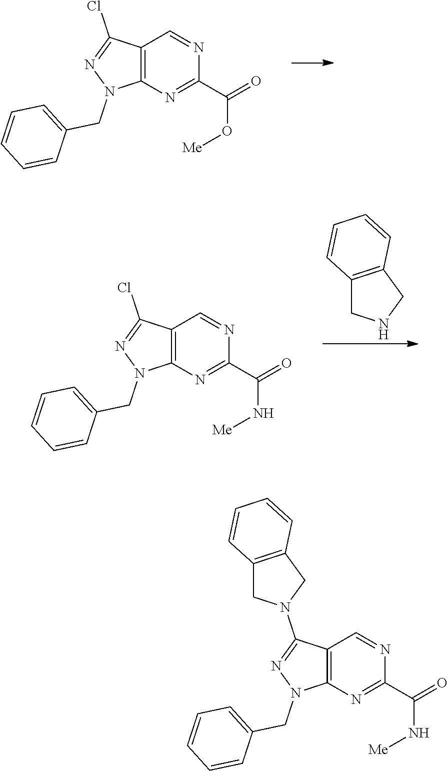 [SMILES: COC(=O)c1ncc2c(Cl)nn(Cc3ccccc3)c2n1 ; CNC(=O)c1ncc2c(Cl)nn(Cc3ccccc3)c2n1 ; c1ccc2c(c1)CNC2 ; CNC(=O)c1ncc2c(N3Cc4ccccc4C3)nn(Cc3ccccc3)c2n1]

### 1-benzyl-3-chloro-N-methyl-1H-pyrazolo[3,4-d]pyrimidine-6-carboxamide

Methyl 1-benzyl-3-chloro-1H-pyrazolo[3,4-d]pyrimidine-6-carboxylate (183 mg, 1 Eq, 605 μmol) was dissolved in methylamine in EtOH (939 mg, 0.99 mL, 20 Wt %, 10 Eq, 6.05 mmol) and heated at 80° C. for one hour. Upon cooling, the crude reaction was concentrated onto silica gel and purified by silica gel column chromatography (0-10% MeOH in DCM) to provide 1-benzyl-3-chloro-N-methyl-1H-pyrazolo[3,4-d]pyrimidine-6-carboxamide (151 mg, 500 μmol, 82.8%).

LCMS (ESI+): m/z 302.0 [M+H]⁺

### 1-benzyl-3-(isoindolin-2-yl)-N-methyl-1H-pyrazolo[3,4-d]pyrimidine-6-carboxamide. Compound 19

1-Benzyl-3-chloro-N-methyl-1H-pyrazolo[3,4-d]pyrimidine-6-carboxamide (50 mg, 1 Eq, 0.17 mmol) and isoindoline (59 mg, 3 Eq, 0.50 mmol) were dissolved in NMP (0.54 mL) and heated at 180° C. in a microwave reactor for 3 hours. Upon cooling, the reaction mixture was loaded directly onto a preparative HPLC (C18 column, 10-100% MeCN in water with 0.1% TFA) to provide 1-benzyl-3-(isoindolin-2-yl)-N-methyl-1H-pyrazolo[3,4-d]pyrimidine-6-carboxamide (30 mg, 78 μmol, 47%).

LCMS: m/z=385.0, LCAP=99% @ 254 nm

¹H NMR: (400 MHz, DMSO-d6) d ppm 9.45 (s, 1H), 9.04-9.02 (m, 1H), 7.51-7.49 (m, 2H), 7.42-7.37 (m, 4H), 7.34-7.32 (m, 4H), 5.62 (s, 2H), 5.02 (s, 4H), 2.92 (d, J=4.8 Hz, 3H).

### Synthesis of Compound 257.

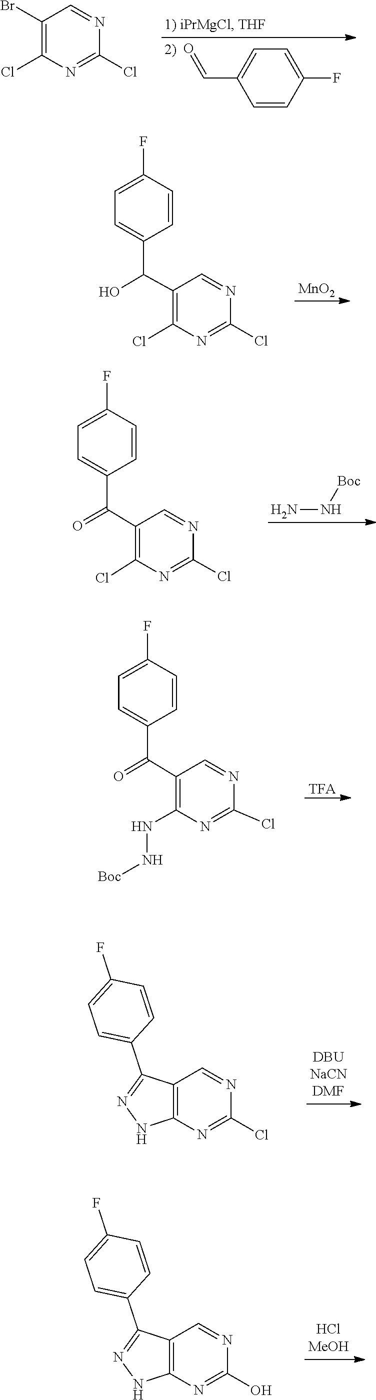

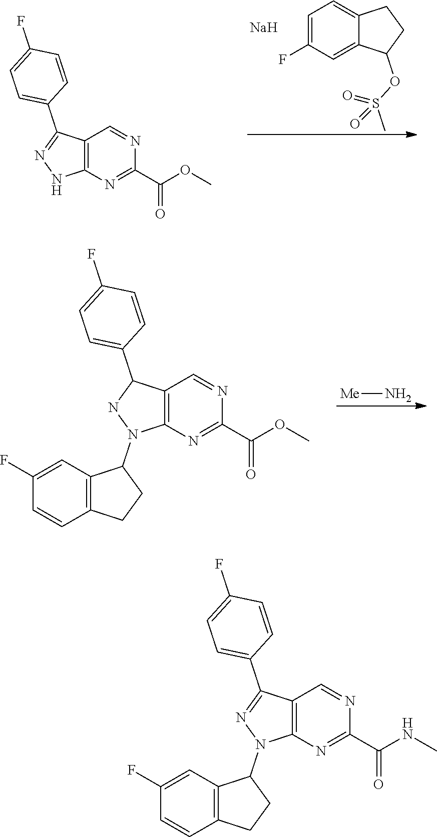

### (2,4-dichloropyrimidin-5-yl)(4-fluorophenyl)methanol

To a solution of 5-bromo-2,4-dichloropyrimidine (13.2 g, 57.4 mmol) in THF (100 mL) at −45° C. was added isopropylmagnesium chloride (1 M in THF, 57.4 mL, 57.4 mmol) dropwise over 20 minutes. The reaction was then stirred at the same temperature for 45 minutes. Then, 4-fluorobenzaldehyde (5.16 g, 41.5 mmol) was added dropwise over 10 minutes, and stirring was continued for 15 minutes. At this time, the cooling bath was removed and the mixture warmed to room temperature over 1 hour. The mixture was diluted with EtOAc (100 mL) and quenched with saturated aqueous ammonium chloride. The layers were separated, and the aqueous layer was extracted with ethyl acetate. The combined organic solution was dried over MgSO₄ and evaporated in vacuo to give (2,4-dichloropyrimidin-5-yl)(4-fluorophenyl)methanol (15.6 g, 57.1 mmol, 99%).

LCMS (ESI+): m/z 273.0 [M+H]⁺

### (2,4-dichloropyrimidin-5-yl)(4-fluorophenyl)methanone

To a solution of (2,4-dichloropyrimidin-5-yl)(4-fluorophenyl)methanol (15.6 g, 57.1 mmol) in dichloromethane (250 mL) was added manganese(IV) oxide (99.2 g, 1142 mmol), and the mixture was stirred for 2 days at room temperature. The reaction mixture was filtered through celite and eluted further with EtOAc. The filtrate was concentrated under reduced pressure to give (2,4-dichloropyrimidin-5-yl)(4-fluorophenyl)methanone (crude, 15.4 g, 56.8 mmol).

LCMS (ESI+): m/z 271.0 [M+H]⁺

### 6-chloro-3-(4-fluorophenyl)-1H-pyrazolo[3,4-d]pyrimidine

To a solution of crude 2,4-dichloro-5-(4-fluorobenzoyl)pyrimidine (15.4 g, 56.8 mmol) and ethylbis(propan-2-yl)amine (8.80 g, 68.1 mmol) in THF (100 mL) was added (tert-butoxy)carbohydrazide (7.50 g, 56.8 mmol) at 0° C. After stirring at 0° C. for 2 hours, the mixture was allowed to stir at room temperature for 16 hours. The mixture was diluted with 0.5 M aqueous HCl (200 mL) and extracted with ethyl acetate. The organic layer was washed with brine and dried over sodium sulfate. The organic solution was concentrated under reduced pressure. The residue was purified by column chromatography (silica gel, 200 g, 40% EtOAc in heptane) to give tert-butyl 2-(2-chloro-5-(4-fluorobenzoyl)pyrimidin-4-yl)hydrazinecarboxylate (7.17 g, 34.4%) as a pale yellow foam. A solution of tert-butyl 2-(2-chloro-5-(4-fluorobenzoyl)pyrimidin-4-yl)hydrazine-1-carboxylate (7.17 g, 19.5 mmol) in dichloromethane (80 mL) and trifluoroacetic acid (20 mL) was refluxed for 3 hours. After cooling to room temperature, the mixture was concentrated under reduced pressure. The solid was suspended with ethyl acetate and collected by vacuum filtration to afford 6-chloro-3-(4-fluorophenyl)-1H-pyrazolo[3,4-d]pyrimidine (1.99 g, 41.1%) as a pale yellow solid.

¹H NMR (400 MHz, DMSO-d6) δ ppm 14.42 (s, 1H), 9.61 (s, 1H), 8.71-8.14 (m, 2H), 7.42-7.37 (m, 2H).

### 3-(4-fluorophenyl)-1H-pyrazolo[3,4-d]pyrimidine-6-carbonitrile

A mixture of 6-chloro-3-(4-fluorophenyl)-1H-pyrazolo[3,4-d]pyrimidine (0.50 g, 2.01 mmol), sodium cyanide (197 mg, 4.02 mmol), and 1,4-diazabicyclo[2.2.2]octane (22.5 mg, 0.201 mmol) in DMF (4 mL) was heated at 130° C. for 1 hour. After cooling to room temperature, the mixture was poured into brine (400 mL), and the insoluble solid was collected by vacuum filtration. The filtrate was extracted with ethyl acetate 3 times. The combined organic layers were dried over sodium sulfate and concentrated under reduced pressure. The residue and insoluble solid were combined and washed with diethyl ether. The material was then dried under reduced pressure to afford crude 3-(4-fluorophenyl)-1H-pyrazolo[3,4-d]pyrimidine-6-carbonitrile (480 mg, 100%).

LCMS (ESI+): m/z 240.2 [M+H]⁺

### methyl 3-(4-fluorophenyl)-1H-pyrazolo[3,4-d]pyrimidine-6-carboxylate

3-(4-Fluorophenyl)-1H-pyrazolo[3,4-d]pyrimidine-6-carbonitrile (509 mg, 2.12 mmol) was dissolved in hydrochloric acid in MeOH (3 M, 15 mL) and stirred at 55° C. for 16 hours. Upon cooling, the solvent was removed in vacuo. The resulting solid was taken up in EtOAc and collected by vacuum filtration to provide methyl 3-(4-fluorophenyl)-1H-pyrazolo[3,4-d]pyrimidine-6-carboxylate (584 mg, 100%) as a brown solid.

LCMS (ESI+): m/z 273.2 [M+H]⁺

### 6-fluoro-2,3-dihydro-1H-inden-1-yl methanesulfonate

To a mixture of 6-fluoro-2,3-dihydro-1H-inden-1-ol (300 mg, 1 Eq, 1.97 mmol) and triethylamine (299 mg, 0.41 mL, 1.5 Eq, 2.96 mmol) in DCE (5 mL) was added mathensulfonyl chloride (271 mg, 184 μL, 1.2 Eq, 2.37 mmol) at 0° C. The mixture was stirred for 1 hour at 0° C. and then treated with saturated aqueous ammonium chloride. The mixture was extracted with dichloromethane and the layers were separated. The organic solution was dried over sodium sulfate and concentrated under reduced pressure to give 6-fluoro-2,3-dihydro-1H-inden-1-yl methanesulfonate (454 mg, 1.97 mmol, 100%) as a pale-yellow oil. Material was used in the next step without further purification.

### methyl 1-(6-fluoro-2,3-dihydro-1H-inden-1-yl)-3-(4-fluorophenyl)-1H-pyrazolo[3,4-d]pyrimidine-6-carboxylate

To a suspension of methyl 3-(4-fluorophenyl)-1H-pyrazolo[3,4-d]pyrimidine-6-carboxylate (50 mg, 1 Eq, 0.18 mmol) in THF (3 mL) was added sodium hydride (5.7 mg, 1.3 Eq, 0.24 mmol) at 0° C. After 30 minutes, 6-fluoro-2,3-dihydro-1H-inden-1-yl methanesulfonate (85 mg, 2 Eq, 0.37 mmol) in THF (1 mL) was added, and the mixture was stirred for 5 hours at 0° C. The reaction was quenched with saturated aqueous ammonium chloride. The mixture was extracted with ethyl acetate, dried over sodium sulfate, and concentrated under reduced pressure. The residue was purified by column chromatography (silica gel, 10-50% EtOAc in heptane) to afford methyl 1-(6-fluoro-2,3-dihydro-1H-inden-1-yl)-3-(4-fluorophenyl)-1H-pyrazolo[3,4-d]pyrimidine-6-carboxylate (40.1 mg, 98.7 μmol, 54%) as a white solid.

LCMS: m/z=407.1 [M+]+

¹H NMR (400 MHz, CHLOROFORM-d) δ ppm 9.52 (s, 1H), 7.89-7.96 (m, 2H), 7.28-7.33 (m, 1H), 7.19-7.25 (m, 2H), 6.93-6.99 (m, 1H), 6.76 (t, J=7.10 Hz, 1H), 6.69 (dd, J=2.20, 8.56 Hz, 1H), 4.14 (s, 3H), 3.35-3.44 (m, 1H), 2.94-3.15 (m, 2H), 2.77-2.85 (m, 2H).

### 1-(6-fluoro-2,3-dihydro-1H-inden-1-yl)-3-(4-fluorophenyl)-N-methyl-1H-pyrazolo[3,4-d]pyrimidine-6-carboxamide. Compound 257

A mixture of methyl 1-(6-fluoro-2,3-dihydro-1H-inden-1-yl)-3-(4-fluorophenyl)-1H-pyrazolo[3,4-d]pyrimidine-6-carboxylate (20.0 mg, 1 Eq, 49.2 μmol) and methylamine in ethanol (2 mL, 33 wt %) was heated at 100° C. under microwave irradiation for 3 hours. Upon cooling, the crude solution was concentrated, and the residue was purified by column chromatography (silica gel, 20-100% EtOAc in heptane) to afford 1-(6-fluoro-2,3-dihydro-1H-inden-1-yl)-3-(4-fluorophenyl)-N-methyl-1H-pyrazolo[3,4-d]pyrimidine-6-carboxamide (9.5 mg, 23 μmol, 48%) as a white solid.

LCMS: m/z=406 [M+H]+

¹H NMR (400 MHz, CHLOROFORM-d) δ ppm 9.44 (s, 1H), 8.20 (d, J=5.10 Hz, 1H),

7.88-7.97 (m, 2H), 7.28-7.34 (m, 1H), 7.17-7.26 (m, 2H), 6.95-7.02 (m, 1H), 6.79 (t, J=7.20 Hz, 1H), 6.66 (dd, J=2.51, 8.40 Hz, 1H), 3.32-3.41 (m, 1H), 3.15 (d, J=5.10 Hz, 3H), 3.00-3.13 (m, 1H), 2.72-2.85 (m, 2H)

### Synthesis of Compound 262

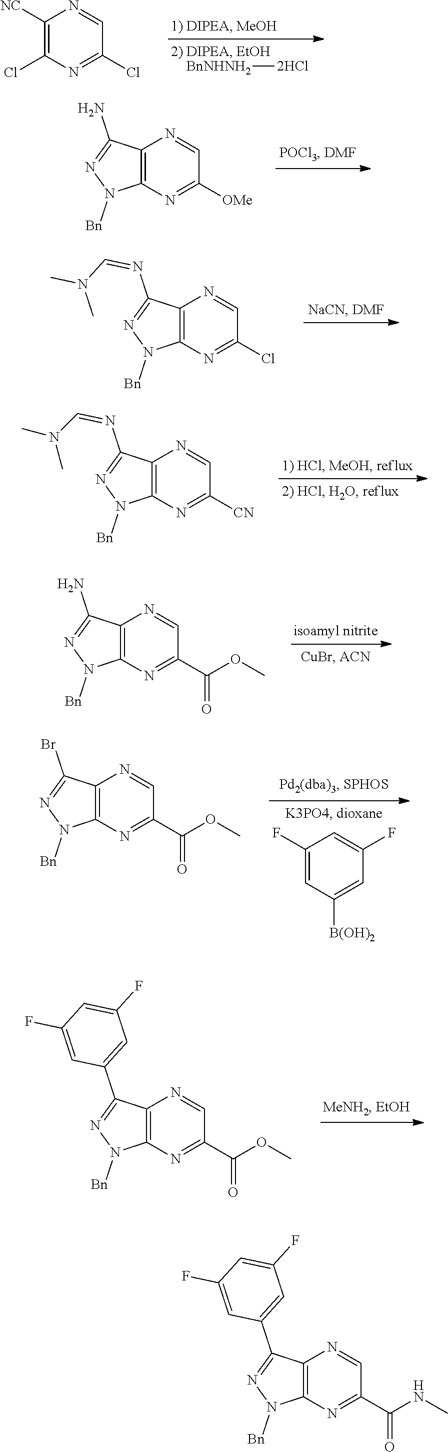

### 1-benzyl-6-methoxy-1H-pyrazolo[3,4-b]pyrazin-3-amine

3,5-Dichloropyrazine-2-carbonitrile (50.0 g, 287 mmol, 1.0 eq) was dissolved in MeOH (625 mL). Then, at 0° C., DIPEA (59.6 mL, 330 mmol, 1.15 eq) was added over 5 minutes. The reaction was stirred for 16 hours while warming to room temperature. The reaction was then concentrated to dryness and dried for 5 minutes on the high vacuum pump. The crude material was dissolved in EtOH (625 mL) and benzylhydrazine dihydrochloride (98.1 g, 503 mmol, 1.75 eq) was added. DIPEA (260 mL, 1492 mmol, 5.25 eq.) was then added slowly using an addition funnel. The reaction was stirred at room temperature until the mixture became homogeneous. The reaction was then heated at reflux for 3 hours. A seed of desired product was then dropped in the reaction while hot. The hot plate was turned off and the mixture was stirred while slowly cooling to room temperature for 16 hours. The precipitate was collected by vacuum filtration and washed with cold ethanol. The powder was dried for 16 hours under vacuum to afford 1-benzyl-6-methoxy-1H-pyrazolo[3,4-b]pyrazin-3-amine as a tan powder (40.3 g, 54.9%).

¹H NMR (600 MHz, DMSO) δ ppm 7.97 (s, 1H), 7.34-7.28 (m, 2H), 7.27-7.21 (m, 3H), 5.67 (s, 2H), 5.26 (s, 2H), 3.98 (s, 3H).

### (Z)—N′-(1-benzyl-6-chloro-1H-pyrazolo[3,4-b]pyrazin-3-yl)-N,N-dimethylformimidamide

Phosphorous oxychloride (32.3 mL, 0.353 mol, 5.0 eq) was added dropwise to DMF (109 mL, 1.41 mol, 20.0 eq) at 0° C. Then, at 0° C., 1-benzyl-6-methoxy-1H-pyrazolo[3,4-b]pyrazin-3-amine (18.0 g, 0.071 mol, 1.0 eq) was added portion-wise as a solid. After the addition, the reaction was warmed to room temperature and then heated at 110° C. for 16 hours. The reaction was cooled to room temperature and then poured slowly onto ice mixed with sodium carbonate (112.1 g, 1.058 mol, 15.0 eq). Ethyl acetate was added to the cold aqueous mixture and the layers were separated. The aqueous layer was extracted with ethyl acetate (3×). The combined organic layers were washed with water, dried over sodium sulfate, and concentrated to afford (Z)—N′-(1-benzyl-6-chloro-1H-pyrazolo[3,4-b]pyrazin-3-yl)-N,N-dimethylformimidamide (16.6 g, 75%).

¹H NMR (600 MHz, DMSO) δ ppm 8.63 (s, 1H), 8.55 (s, 1H), 7.35-7.30 (m, 2H), 7.30-7.26 (m, 1H), 7.26-7.22 (m, 2H), 5.46 (s, 2H), 3.08 (s, 3H), 3.00 (s, 3H).

### (Z)—N′-(1-benzyl-6-cyano-1H-pyrazolo[3,4-b]pyrazin-3-yl)-N,N-dimethylformimidamide

To a stirred solution of (Z)—N′-(1-benzyl-6-chloro-1H-pyrazolo[3,4-b]pyrazin-3-yl)-N,N-dimethylformimidamide (12.5 g, 0.04 mol, 1.0 eq) in DMF (100 mL) was added sodium cyanide (2.58 g, 0.052 mol, 1.3 eq). The reaction was stirred at 100° C. for 16 hours. The reaction was cooled to room temperature and water (300 mL) was added. The slurry was stirred for 1 hour before the solid was collected by vacuum filtration. The powder was washed with water and heptane to afford (Z)—N′-(1-benzyl-6-cyano-1H-pyrazolo[3,4-b]pyrazin-3-yl)-N,N-dimethylformimidamide as a white powder (11.1 g, 91.2%).

¹H NMR (600 MHz, DMSO) δ 8.98 (s, 1H), 8.67 (s, 1H), 7.35-7.31 (m, 2H), 7.30-7.25 (m, 3H), 5.55 (s, 2H), 3.09 (s, 3H), 3.02 (s, 3H).

### methyl 3-amino-1-benzyl-1H-pyrazolo[3,4-b]pyrazine-6-carboxylate

(Z)—N′-(1-Benzyl-6-cyano-1H-pyrazolo[3,4-b]pyrazin-3-yl)-N,N-dimethylformimidamide (9.3 g, 30.5 mmol, 1.0 eq) was mixed with 3 N methanolic HCl (102 mL, 305 mmol, 10.0 eq), and the mixture was heated at reflux for 6 hours. Then, water was added (20.0 mL, 944 mmol, 31.0 eq) and the reaction was heated at reflux for an additional 16 hours. The reaction was cooled to room temperature and partitioned between DCM and a saturated aqueous solution of sodium bicarbonate. The layers were separated, and the aqueous layer was extracted with DCM 3 times. The combined organic layers were washed with brine, dried over sodium sulfate, filtered, and concentrated under reduced vacuum to afford methyl 3-amino-1-benzyl-1H-pyrazolo[3,4-b]pyrazine-6-carboxylate as a white powder. (6.33 g, 73.4%).

¹H NMR (600 MHz, DMSO) δ ppm 8.97 (s, 1H), 7.33-7.29 (m, 2H), 7.28-7.24 (m, 1H), 7.22-7.18 (m, 2H), 6.15 (s, 2H), 5.44 (s, 2H), 3.95 (s, 3H).

### methyl 1-benzyl-3-bromo-1H-pyrazolo[3,4-b]pyrazine-6-carboxylate

To a suspension of methyl 3-amino-1-benzyl-1H-pyrazolo[3,4-b]pyrazine-6-carboxylate (5.80 g, 20.5 mmol, 1.0 eq) and copper(II) bromide (4.80 g, 21.5 mmol, 1.05 eq) in dry acetonitrile (249 mL) was added isoamylnitrite dropwise (3.58 mL, 26.6 mmol, 1.3 eq). The reaction was stirred for 16 hours at room temperature and filtered through celite eluting with DCM. This solution was then concentrated onto silica gel and purified by silica gel column chromatography (0-30% EtOAc in 20% DCM in hexanes) to afford methyl 1-benzyl-3-bromo-1H-pyrazolo[3,4-b]pyrazine-6-carboxylate as a tan powder (3.89 g, 54.7%).

¹H NMR (600 MHz, DMSO) δ 9.28 (s, 1H), 7.37-7.32 (m, 2H), 7.32-7.27 (m, 3H), 5.75 (s, 2H), 4.00 (s, 3H).

### methyl 1-benzyl-3-(3,5-difluorophenyl)-1H-pyrazolo[3,4-b]pyrazine-6-carboxylate

Methyl 1-benzyl-3-bromo-1H-pyrazolo[3,4-b]pyrazine-6-carboxylate (5.0 g, 14.4 mmol, 1.0 eq), Pd₂(dba)₃ (0.659 g, 0.72 mmol, 0.050 eq), potassium phosphate tribasic (9.17 g, 43.2 mmol, 3.0 eq), SPHOS (1.18 g, 2.88 mmol, 0.2 eq), and (3,5-difluorophenyl)boronic acid (4.55 g, 28.8 mmol, 2.0 eq) were combined in a RBF and dioxane (80 mL) was added. The reaction was degassed with argon for 5 minutes and then heated at 80° C. for 3 hours. The reaction was cooled to room temperature and diluted with ethyl acetate and water. The layers were separated, and the aqueous layer was extracted with ethyl acetate two times. The combined organics layers were dried over sodium sulfate, filtered, and concentrated onto silica. This crude material was then purified with silica gel column chromatography (220 g, 0-50% EtOAc in hexanes) to afford methyl 1-benzyl-3-(3,5-difluorophenyl)-1H-pyrazolo[3,4-b]pyrazine-6-carboxylate (3.80 g, 69%).

¹H NMR (600 MHz, DMSO) δ ppm 9.38 (s, 1H), 8.05 (d, J=6.3 Hz, 2H), 7.43-7.37 (m, 1H), 7.37-7.28 (m, 5H), 5.85 (s, 2H), 4.00 (s, 3H).

### 1-benzyl-3-(3,5-difluorophenyl)-N-methyl-1H-pyrazolo[3,4-b]pyrazine-6-carboxamide. Compound 262

Methyl 1-benzyl-3-(3,5-difluorophenyl)-1H-pyrazolo[3,4-b]pyrazine-6-carboxylate (10.5 g, 0.028 mol, 1.0 eq) was treated with methylamine (33% in ethanol, 207 mL, 1.66 mol, 60.0 eq). The slurry was stirred for 4 hours then concentrated under vacuum to approximately 30 mL. The slurry was filtered, and the solid was washed with ethanol. The powder was triturated with hot ethanol and allowed to settle overnight while cooling to room temperature. The solid was then collected by vacuum filtration to afford 1-benzyl-3-(3,5-difluorophenyl)-N-methyl-1H-pyrazolo[3,4-b]pyrazine-6-carboxamide as a yellow powder. (9.02 g 86.1%).

¹H NMR (300 MHz, DMSO) δ 9.34 (s, 1H), 9.19-9.09 (m, 1H), 8.07-7.96 (m, 2H), 7.43 (d, J=7.3 Hz, 2H), 7.38-7.33 (m, 3H), 7.33-7.28 (m, 1H), 5.86 (s, 2H), 2.92 (d, J=4.8 Hz, 3H).

m/z 380.6 (M+H)⁺.

### Synthesis of Compound 263.

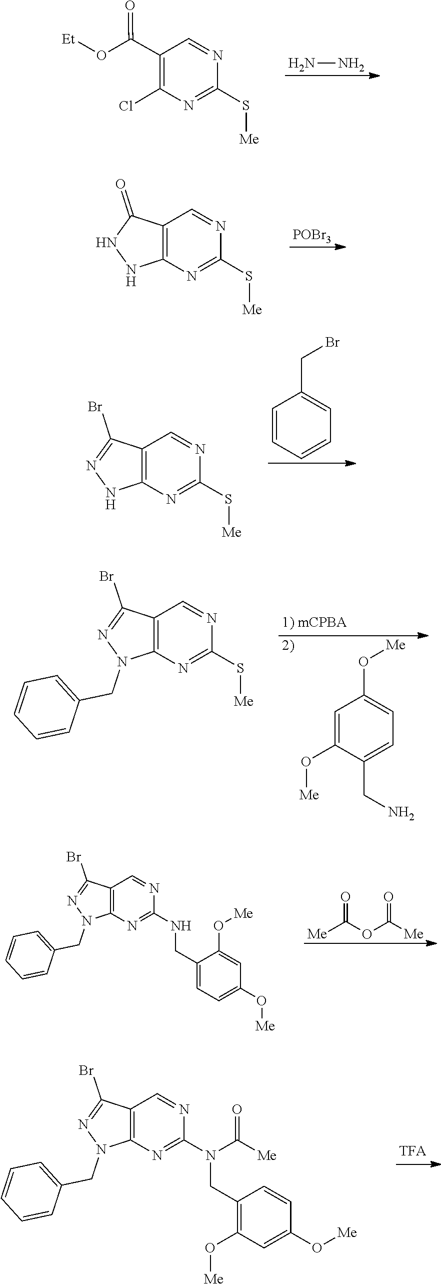

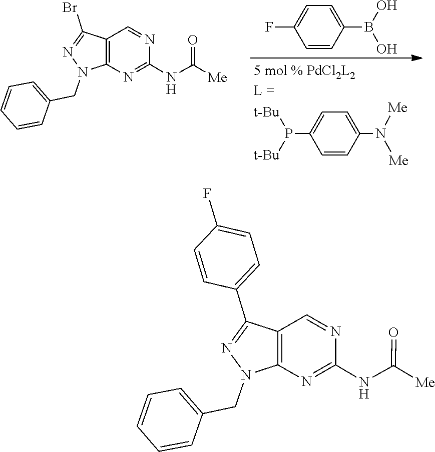 [SMILES: CC(=O)Nc1ncc2c(Br)nn(Cc3ccccc3)c2n1 ; OB(O)c1ccc(F)cc1 ; CN(C)c1ccc(P(C(C)(C)C)C(C)(C)C)cc1 ; CC(=O)Nc1ncc2c(-c3ccc(F)cc3)nn(Cc3ccccc3)c2n1]

### 6-(methylthio)-1H-pyrazolo[3,4-d]pyrimidin-3(2H)-one

Ethyl 4-chloro-2-(methylsulfanyl)pyrimidine-5-carboxylate (15 g, 63.1 mmol) was dissolved in ethanol (225 mL). Hydrazine hydrate (9.84 g, 125 mmol) was added drop wise over 30 minutes. The reaction solution became thick and cloudy. The solution was concentrated under reduced pressure to remove all ethanol. The resulting white solid was then dissolved in 200 mL of a 10% aqueous KOH solution and heated at reflux for 15 minutes. After the reaction was cooled to room temperature, the solution was acidified with a 25% solution of acetic acid in water until the PH reached 4. The solution became yellow as the product crashed out of solution, and the mixture was allowed to sit for 16 hours to allow all product to precipitate out. The precipitate was collected by vacuum filtration, washed with water, and dried under reduced pressure to provide 6-(methylthio)-1H-pyrazolo[3,4-d]pyrimidin-3(2H)-one (9.21 g, 80.7%).

LCMS (ESI+): m/z 183.1 [M+H]⁺

### 3-bromo-6-(methylthio)-1H-pyrazolo[3,4-d]pyrimidine

6-(Methylsulfanyl)-1H,2H,3H-pyrazolo[3,4-d]pyrimidin-3-one (5.0 g, 27.4 mmol) and phosphoroyl tribromide (15.7 g, 54.8 mmol) were suspended in acetonitrile (50 mL). The reaction mixture was sonicated for thirty minutes and then stirred in a sealed vial at 100° C. overnight. The reaction was carefully quenched with water and the product was extracted with ethyl acetate. The organic layer was dried over sodium sulfate and concentrated under reduced pressure to provide 3-bromo-6-(methylthio)-1H-pyrazolo[3,4-d]pyrimidine (2.50 g, 37.2%).

LCMS (ESI+): m/z 245.9 [M+H]⁺

### 1-benzyl-3-bromo-6-(methylthio)-1H-pyrazolo[3,4-d]pyrimidine

3-Bromo-6-(methylsulfanyl)-1H-pyrazolo[3,4-d]pyrimidine (1.5 g, 6.11 mmol), (chloromethyl)benzene (1.15 g, 9.16 mmol), and cesium carbonate (3.97 g, 12.2 mmol) were dissolved in DMF (10 mL) and stirred at 100° C. for 2 hours. The crude mixture was dry loaded onto silica gel and purified by column chromatography (silica gel, 30 g, 20% EtOAc in heptane) to provide 1-benzyl-3-bromo-6-(methylthio)-1H-pyrazolo[3,4-d]pyrimidine (1.90 g, 93.1%) as a white solid.

LCMS (ESI+): m/z 335.0 [M+H]⁺

### 1-benzyl-3-bromo-N-(2,4-dimethoxybenzyl)-1H-pyrazolo[3,4-d]pyrimidin-6-amine

1-Benzyl-3-bromo-6-(methylsulfanyl)-1H-pyrazolo[3,4-d]pyrimidine (1.9 g, 5.66 mmol) was dissolved in THF (10 mL). 3-Chlorobenzene-1-carboperoxoic acid (2.78 g, 11.3 mmol) was then added. The reaction was stirred at room temperature for 1 hour. The resulting sulfone and sulfoxide mixture was cooled to 0° C., and (2,4-dimethoxyphenyl)methanamine hydrochloride (2.30 g, 11.3 mmol) suspended in 1 mL of THF was added. The reaction was stirred at 80° C. for 1 hour. Upon cooling to room temperature, the reaction mixture was diluted with DCM and washed with saturated aqueous sodium bicarbonate. The organic layer was dried over sodium sulfate and concentrated under reduced pressure to give 1-benzyl-3-bromo-N-(2,4-dimethoxybenzyl)-1H-pyrazolo[3,4-d]pyrimidin-6-amine (2.20 g, 85.6%) as a white solid. The product was used in the next step without further purification.

LCMS (ESI+): m/z 454.1 [M+H]⁺

### N-(1-benzyl-3-bromo-1H-pyrazolo[3,4-d]pyrimidin-6-yl)-N-(2,4-dimethoxybenzyl)acetamide

1-Benzyl-3-bromo-N-[(2,4-dimethoxyphenyl)methyl]-1H-pyrazolo[3,4-d]pyrimidin-6-amine (2.1 g, 4.62 mmol) was dissolved in acetyl acetate (10.8 g, 105 mmol) and stirred at 135° C. for 7 hours. Upon cooling to room temperature, the reaction was concentrated under reduced pressure, mixed with saturated aqueous sodium bicarbonate, and extracted with dichloromethane. The organic layer was dried over sodium sulfate and concentrated under reduced pressure to provide N-(1-benzyl-3-bromo-1H-pyrazolo[3,4-d]pyrimidin-6-yl)-N-(2,4-dimethoxybenzyl)acetamide as an oil (2.29 g, 100%).

LCMS (ESI+): m/z 496.0 [M+H]⁺

### N-(1-benzyl-3-bromo-1H-pyrazolo[3,4-d]pyrimidin-6-yl)acetamide

N-{1-Benzyl-3-bromo-1H-pyrazolo[3,4-d]pyrimidin-6-yl}-N-[(2,4-dimethoxyphenyl)methyl]acetamide (2.29 g, 4.61 mmol) was dissolved in trifluoroacetic acid (10 mL) and stirred at 70° C. for 2 hours. The reaction was concentrated under reduced pressure and carefully mixed with saturated aqueous sodium bicarbonate. The product was extracted with DCM and dry loaded onto silica gel. The product was purified by column chromatography (silica gel, 40 g, 50% acetone in heptane) to provide N-(1-benzyl-3-bromo-1H-pyrazolo[3,4-d]pyrimidin-6-yl)acetamide (954 mg, 60.0%) as a white solid.

LCMS (ESI+): m/z 346.1 [M+H]⁺

### N-(1-benzyl-3-(4-fluorophenyl)-1H-pyrazolo[3,4-d]pyrimidin-6-yl)acetamide. Compound 263

(4-Fluorophenyl)boronic acid (54.2 mg, 388 μmol), N-{1-benzyl-3-bromo-1H-pyrazolo[3,4-d]pyrimidin-6-yl}acetamide (90 mg, 259 μmol), bis(4-(di-tert-butylphosphanyl)-N,N-dimethylaniline) dichloropalladium (9.13 mg, 12.9 μmol), and potassium acetate (76.2 mg, 777 μmol) were degassed in a reaction vial with argon. Degassed butan-1-ol (2 mL) was added to the reaction vial and it was stirred at 70° C. for 2 hours. The reaction was then dry loaded onto silica gel. The reaction mixture was purified by column chromatography (silica gel, 24 g, 50% acetone in heptane) to provide N-(1-benzyl-3-(4-fluorophenyl)-1H-pyrazolo[3,4-d]pyrimidin-6-yl)acetamide (58.0 mg, 62.0%) as a white solid.

¹H NMR (400 MHz, DMSO-d6) d ppm 10.84 (s, 1H), 9.51 (s, 1H), 8.11 (dd, J=5.50, 8.44 Hz, 2H), 7.27-7.40 (m, 7H), 5.60 (s, 2H), 2.28 (s, 3H).

LCMS (ESI+): m/z 362.1 [M+H]⁺

Additional Compounds Synthesized in an Analagous Manner to Compound 263:

| | Exact | LCMS | |
|---|---|---|---|
| Compound | Mass | Found | Assignment |
| 264 | 343.1433 | 343.4 | [M + H]⁺ |
| 265 | 357.159 | 358.3 | [M + H]⁺ |
| 266 | 357.159 | 358.1 | [M + H]⁺ |
| 267 | 362.1291 | 363.0 | [M + H]⁺ |
| 268 | 368.1386 | 369.2 | [M + H]⁺ |
| 269 | 387.1695 | 388.3 | [M + H]⁺ |
| 270 | 391.1445 | 392.1 | [M + H]⁺ |
| 271 | 421.1209 | 422.1 | [M + H]⁺ |

### Synthesis of Compound 272.

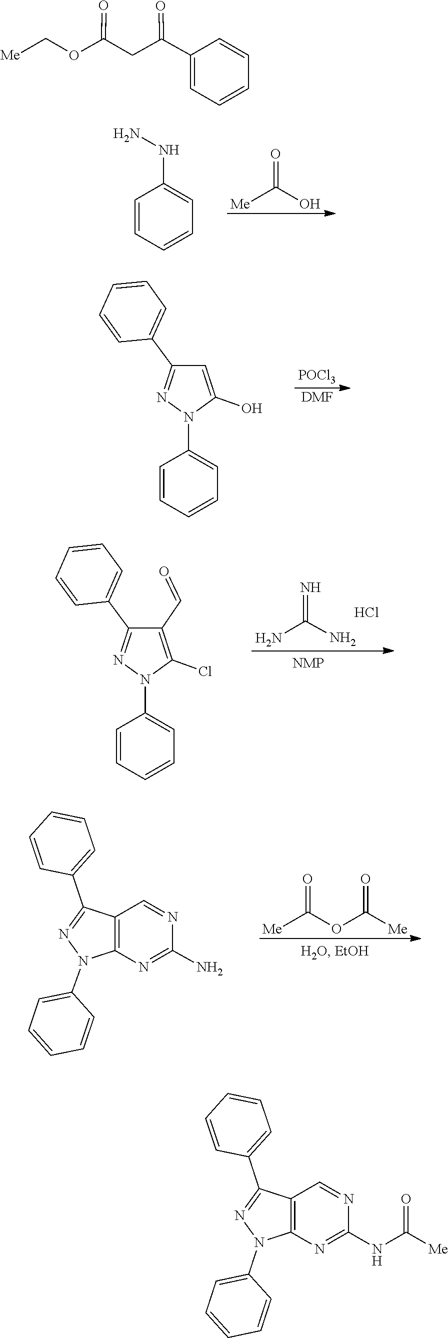

### 1,3-diphenyl-1H-pyrazol-5-ol

Ethyl 3-oxo-3-phenylpropanoate (23.0 g, 120 mmol) and phenylhydrazine (12.9 g, 120 mmol) in acetic acid (60 mL) were heated to reflux for 3 hours. After cooling to room temperature, the residue was poured into water (300 mL) and stirred to form a precipitate. The resulting precipitate was collected by vacuum filtration, washed with water (100 mL×2), and dried under high vacuum for 16 hours. This process provided 1,3-diphenyl-1H-pyrazol-5-ol (27.1 g, 95.7%) as a pale yellow solid.

LCMS (ESI+): m/z 237.1 [M+H]⁺

### 5-chloro-1,3-diphenyl-1H-pyrazole-4-carbaldehyde

To DMF (22.1 g, 303 mmol) was added phosphoroyl trichloride (92.9 g, 606 mmol) at 0° C. After stirring at 0° C. for 30 min, 1,3-diphenyl-1H-pyrazol-5-ol (24 g, 101 mmol) was added to the reaction mixture, and the mixture was stirred at room temperature for 1 hour followed by 120° C. for 3 hours. After cooling to room temperature, the mixture was poured into ice water (800 mL) and stirred for 30 minutes. The formed precipitate was collected by vacuum filtration and dissolved in DCM. The obtained solution was washed with brine and dried over Na₂SO₄. The mixture was concentrated under reduced pressure to give 5-chloro-1,3-diphenyl-1H-pyrazole-4-carbaldehyde (27.0 g, 94.7%) as a brown solid.

LCMS (ESI+): m/z 283.0 [M+H]⁺

### 1,3-diphenyl-1H-pyrazolo[3,4-d]pyrimidin-6-amine

Three reactions were run in sealable reaction tubes with each reaction containing the following reagents. To a solution of 5-chloro-1,3-diphenyl-1H-pyrazole-4-carbaldehyde (2.0 g, 7.07 mmol) in NMP (7 mL) were added guanidine hydrochloride (0.750 g, 7.77 mmol) and potassium carbonate (2.15 g, 15.5 mmol). The resulting reaction mixture was stirred at 190° C. for 18 hours. Each reaction was let cool to ambient temperature and the three reaction tubes were combined and treated with H₂O (60 mL) and stirred for 15 minutes. The resulting brown solid was collected by vacuum filtration, washed with H₂O, and dried under vacuum at 50° C. to give 1,3-diphenyl-1H-pyrazolo[3,4-d]pyrimidin-6-amine as brown solid (0.663 g, 2.30 mmol, 33%).

LCMS (ESI+): m/z 288.2 [M+H]⁺

### N-(1,3-diphenyl-1H-pyrazolo[3,4-d]pyrimidin-6-yl)acetamide. Compound 272

A mixture of 1,3-diphenyl-1H-pyrazolo[3,4-d]pyrimidin-6-amine (0.663 g, 2.30 mmol) in acetyl acetate (12 mL) was heated at 140° C. for 2 hours and a suspension formed. After cooling to room temperature, the resulting mixture was concentrated under reduced pressure. Ethanol (40 mL) and water (10 mL) were then added to the residue, and the mixture was heated to reflux for 16 hours. The resulting precipitate was collected by vacuum filtration and dried under vacuum to give N-(1,3-diphenyl-1H-pyrazolo[3,4-d]pyrimidin-6-yl)acetamide (705 mg, 93.1%) as a colorless solid.

LCMS (ESI+): m/z 330.2 [M+H]⁺

Additional Compounds Prepared in an Analogous Manner to Compound 272:

| | Exact | LCMS | |
|---|---|---|---|
| Compound | Mass | Found | Assignment |
| 273 | 343.1433 | 343.89 | [M + H]⁺ |
| 274 | 345.1226 | 346.1 | [M + H]⁺ |
| 275 | 355.1433 | 356.1 | [M + H]⁺ |

### Synthesis of Compound 276.

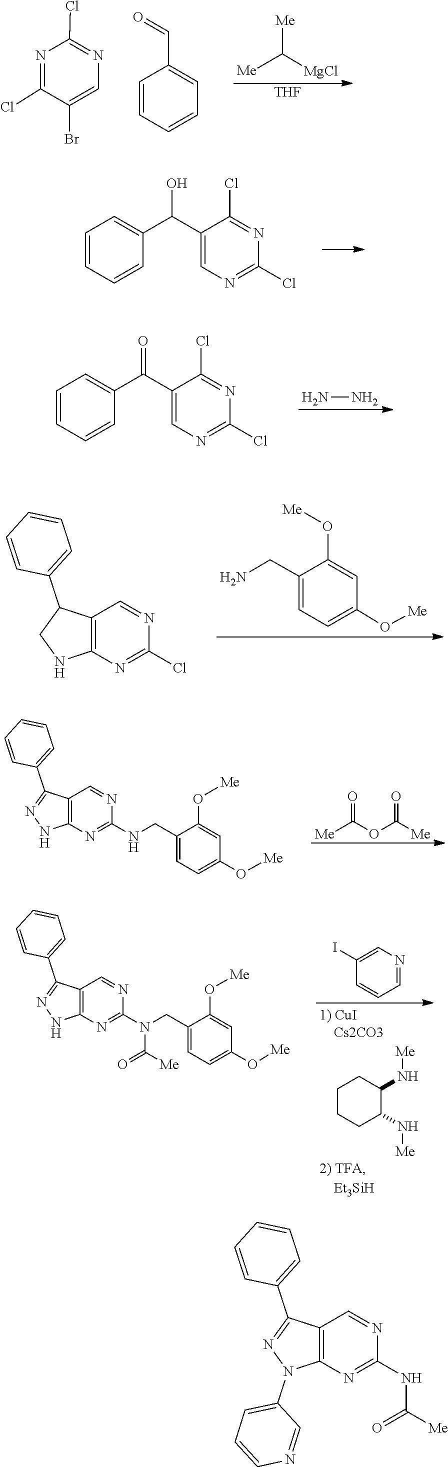

### (2,4-dichloropyrimidin-5-yl)(phenyl)methanol

To a solution of 5-bromo-2,4-dichloropyrimidine (5.12 g, 22.4 mmol) in THF (30 mL) was added 2 M isopropyl magnesium chloride in THF (11.5 mL, 23.0 mmol) dropwise at −35° C. After stirring for 45 minutes at the same temperature, benzaldehyde (2.37 g, 22.4 mmol) was added, and the mixture was warmed to 0° C. for 1 hour. The resulting mixture was quenched with water (10 mL) and then filtered through celite. The filtrate was dried over Na₂SO₄ and concentrated under reduced pressure. The residue was purified by column chromatography (silica gel, 220 g, 40% EtOAc in heptane) to give (2,4-dichloropyrimidin-5-yl)(phenyl)methanol (4.91 g, 85.9%) as a colorless oil.

LCMS (ESI+): m/z 255.1 [M+H]⁺

### (2,4-dichloropyrimidin-5-yl)(phenyl)methanone

To a mixture of (2,4-dichloropyrimidin-5-yl)(phenyl)methanol (4.90 g, 19.2 mmol) in

DCM (150 mL) was added manganese(IV) dioxide (19.9 g, 230 mmol). After stirring for 16 hours at room temperature, the mixture was filtered through celite and then further eluted with ethyl acetate. The filtrate was concentrated under reduced pressure. The residue was purified by column chromatography (silica gel, 240 g, 20% EtOAc in heptane) to give (2,4-dichloropyrimidin-5-yl)(phenyl)methanone (3.08 g, 63.5%) as a colorless viscous oil.

LCMS (ESI+): m/z 252.9 [M+H]⁺

### 6-chloro-3-phenyl-1H-pyrazolo[3,4-d]pyrimidine

To a solution of 5-benzoyl-2,4-dichloropyrimidine (1.55 g, 6.12 mmol) in THF (20 mL) was added hydrazine hydrate (367 mg, 7.34 mmol) at 0° C. After stirring for 10 minutes at room temperature, the resulting mixture was heated at 50° C. for 1 hour. After cooling to room temperature, additional hydrazine hydrate (183 mg, 3.67 mmol) was added to the reaction mixture. After stirring for 1 hour at 50° C., the mixture was cooled to room temperature. The mixture was concentrated under reduced pressure. The residue was suspended with water and methanol, and the resulting precipitate was collected by vacuum filtration. The precipitate was dried under vacuum to give 6-chloro-3-phenyl-1H-pyrazolo[3,4-d]pyrimidine (1.30 g, 92.1%) as an yellow solid.

LCMS (ESI+): m/z 231.1 [M+H]⁺

### N-(2,4-dimethoxybenzyl)-3-phenyl-1H-pyrazolo[3,4-d]pyrimidin-6-amine

A mixture of 6-chloro-3-phenyl-1H-pyrazolo[3,4-d]pyrimidine (1.3 g, 5.63 mmol), (2,4-dimethoxyphenyl)methanamine hydrochloride (2.28 g, 11.2 mmol), and ethylbis(propan-2-yl)amine (2.17 g, 16.8 mmol) in 1-methylpyrrolidin-2-one (10 mL) was heated at 150° C. for 6 hours. After cooling to room temperature, the mixture was diluted with water, and the formed precipitate was collected by vacuum filtration. This solid was suspended in ethanol and then collected by vacuum filtration to give N-(2,4-dimethoxybenzyl)-3-phenyl-1H-pyrazolo[3,4-d]pyrimidin-6-amine (1.69 g, 83.2%) as an orange solid.

LCMS (ESI+): m/z 362.1 [M+H]⁺

### N-(2,4-dimethoxybenzyl)-N-(3-phenyl-1H-pyrazolo[3,4-d]pyrimidin-6-yl)acetamide

A mixture of N-[(2,4-dimethoxyphenyl)methyl]-3-phenyl-1H-pyrazolo[3,4-d]pyrimidin-6-amine (0.733 g, 2.02 mmol) in acetyl acetate (6 mL) was heated overnight at 140° C. After cooling to room temperature, the mixture was concentrated under reduced pressure. Methanol (6 mL), THF (6 mL), 6 M aqueous sodium hydroxide (3 mL), and water (3 mL) were added to the mixture, and it was stirred for 3 hours at room temperature. The resulting mixture was diluted with 1 N aqueous HCl (30 mL) and water (30 mL). The mixture was extracted with ethyl acetate, and then the organic layer was dried over Na₂SO₄. The mixture was concentrated under reduced pressure. The residue was purified by column chromatography (silica gel, 80 g, 50% EtOAc in heptane) to give N-(2,4-dimethoxybenzyl)-N-(3-phenyl-1H-pyrazolo[3,4-d]pyrimidin-6-yl)acetamide (683 mg, 83.9%) as a colorless solid.

LCMS (ESI+): m/z 404.1 [M+H]⁺

### N-(3-phenyl-1-(pyridin-3-yl)-1H-pyrazolo[3,4-d]pyrimidin-6-yl)acetamide. Compound 276

A mixture of N-[(2,4-dimethoxyphenyl)methyl]-N-{3-phenyl-1H-pyrazolo[3,4-d]pyrimidin-6-yl}acetamide (80 mg, 0.198 mmol), 3-iodopyridine (81.2 mg, 0.396 mmol), cesium carbonate (193 mg, 0.595 mmol), (1R,2R)—N1,N2-dimethylcyclohexane-1,2-diamine (8.45 mg, 0.0595 mmol), and copper(I) iodide (11.3 mg, 0.0595 mmol) in dioxane (2 mL) was heated for 16 hours at 120° C. Upon cooling to room temperature, the mixture was concentrated under reduced pressure, and the resulting residue was diluted with ethyl acetate. The mixture was filtered through celite and then the filtrate was concentrated under reduced pressure. The residue was diluted with trifluoroacetic acid (3 mL) and triethylsilane (0.1 mL) and then refluxed for 2 hours. After cooling to room temperature, the mixture was concentrated under reduced pressure. The residue was diluted with saturated aqueous sodium bicarbonate and then extracted with DCM. The organic layer was dried over sodium sulfate and concentrated under reduced pressure. The residue was recrystallized with methanol/dichloromethane twice to give N-(3-phenyl-1-(pyridin-3-yl)-1H-pyrazolo[3,4-d]pyrimidin-6-yl)acetamide (21.8 mg, 33.3%) as a colorless solid.

LCMS (ESI+): m/z 331.1 [M+H]⁺

### Synthesis of Compound 42.

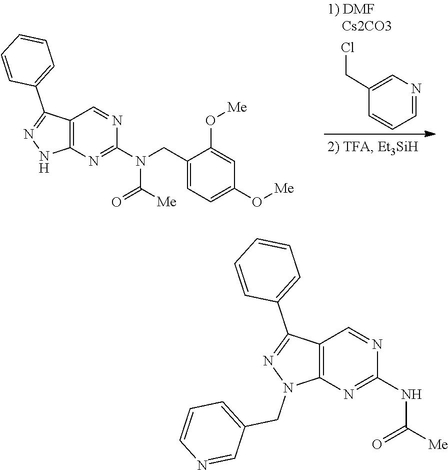

### N-(3-phenyl-1-(pyridin-3-ylmethyl)-1H-pyrazolo[3,4-d]pyrimidin-6-yl)acetamide. Compound 42

A mixture of N-[(2,4-dimethoxyphenyl)methyl]-N-{3-phenyl-1H-pyrazolo[3,4-d]pyrimidin-6-yl}acetamide (80 mg, 0.198 mmol), 3-(chloromethyl)pyridine hydrochloride (65.0 mg, 0.396 mmol), and cesium carbonate (258 mg, 0.793 mmol) in DMF (1.5 mL) was stirred for 16 hours at room temperature. The reaction mixture was diluted with water and then extracted with ethyl acetate. The organic layer was washed with water and dried over sodium sulfate. The mixture was concentrated under reduced pressure. The residue was diluted with trifluoroacetic acid (3 mL) and triethylsilane (0.1 mL) and then refluxed for 2 hours. After cooling to room temperature, the mixture was concentrated under reduced pressure. The residue was diluted with saturated aqueous sodium bicarbonate and extracted with DCM. The organic layer was dried over sodium sulfate and concentrated under reduced pressure. The residue was resolidified with methanol/dichloromethane twice to give N-(3-phenyl-1-(pyridin-3-ylmethyl)-1H-pyrazolo[3,4-d]pyrimidin-6-yl)acetamide (40.4 mg, 59.2%) as a colorless solid.

LCMS (ESI+): m/z 345.2 [M+H]⁺

Additional Compounds Prepared in an Analogous Manner to Compound 42:

| | Exact | LCMS | |
|---|---|---|---|
| Compound | Mass | Found | Assignment |
| 39 | 267.112 | 268.3 | [M + H]⁺ |
| 277 | 281.1277 | 282.2 | [M + H]⁺ |
| 278 | 344.1386 | 345.2 | [M + H]⁺ |
| 279 | 344.1386 | 345.2 | [M + H]⁺ |
| 280 | 349.1903 | 350.3 | [M + H]⁺ |
| 281 | 361.1339 | 362.1 | [M + H]⁺ |

### Synthesis of Compound 282.

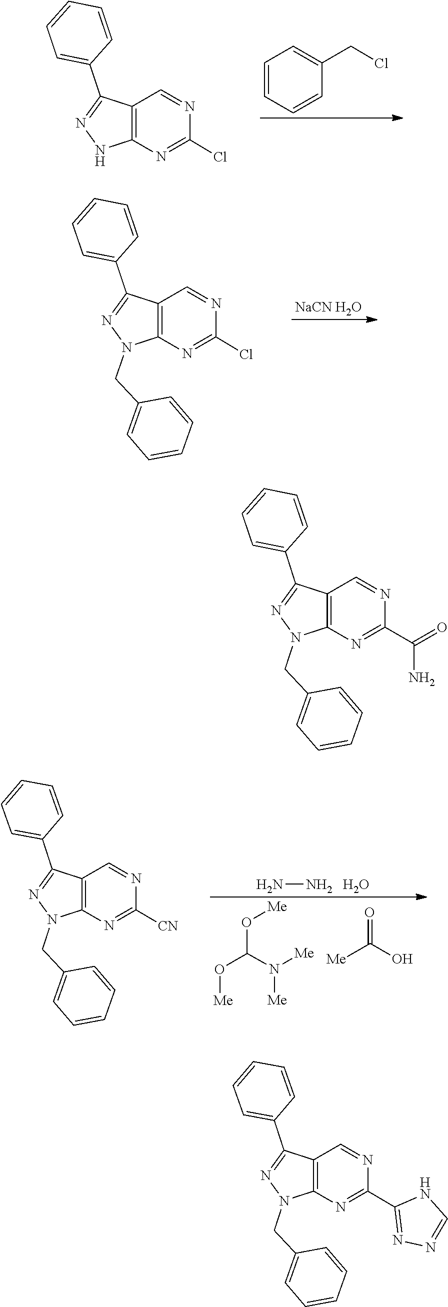

### 6-chloro-3-phenyl-1H-pyrazolo[3,4-d]pyrimidine

Synthesized according to the procedure outlined for 6-chloro-3-(4-fluorophenyl)-1H-pyrazolo[3,4-d]pyrimidine.

LCMS (ESI+): m/z 231.1 [M+H]⁺

### 1-benzyl-6-chloro-3-phenyl-1H-pyrazolo[3,4-d]pyrimidine

To a stirring solution of 6-chloro-3-phenyl-1H-pyrazolo[3,4-d]pyrimidine (15 g, 65.0 mmol) and cesium carbonate (63.5 g, 195 mmol) in DMF (80 mL) at 0° C. was added (chloromethyl)benzene (12.3 g, 97.5 mmol). The reaction was warmed to 50° C. and stirred for 6 hours. Upon cooling to room temperature, the reaction mixture was diluted with water and EtOAc. The organic layer was separated, washed with brine, dried over magnesium sulfate, and concentrated in vacuo. The residue was purified by column chromatography (silica gel, 80 g, 5% MeOH in DCM) to give 1-benzyl-6-chloro-3-phenyl-1H-pyrazolo[3,4-d]pyrimidine (5.60 g, 26.9%) as a pale yellow solid.

LCMS (ESI+): m/z 321.2 [M+H]⁺

### 1-benzyl-3-phenyl-1H-pyrazolo[3,4-d]pyrimidine-6-carboxamide

To a stirring solution of 1,4-diazabicyclo[2.2.2]octane (76.1 mg, 679 μmol) and sodium cyanide (495 mg, 10.1 mmol) in water (15 mL) and DMSO (40 mL) was added 1-benzyl-6-chloro-3-phenyl-1H-pyrazolo[3,4-d]pyrimidine (2.18 g, 6.79 mmol) slowly added at room temperature. The reaction mixture was then heated to 90° C. for two days. Upon cooling to room temperature, the reaction mixture was diluted with water and EtOAc. The organic layer was separated, washed with brine, and concentrated in vacuo. The resulting solid was loaded onto silica gel and purified by column chromatography (silica gel, 40 g, 5% MeOH in DCM) to give 1-benzyl-3-phenyl-1H-pyrazolo[3,4-d]pyrimidine-6-carbonitrile (429 mg, 20.3%) as a pale yellow solid and 1-benzyl-3-phenyl-1H-pyrazolo[3,4-d]pyrimidine-6-carboxamide (827 mg, 37.0%) as a brown solid.

LCMS (ESI+): m/z 330.2 [M+H]⁺

### 1-benzyl-3-phenyl-6-(4H-1,2,4-triazol-3-yl)-1H-pyrazolo[3,4-d]pyrimidine. Compound 282

1-Benzyl-3-phenyl-1H-pyrazolo[3,4-d]pyrimidine-6-carboxamide (70 mg, 212 μmol) was dissolved in (dimethoxymethyl)dimethylamine (1 mL) and heated to 100° C. for two hours. Upon cooling to room temperature, the solvent was removed in vacuo, and the resulting solid was redissolved in acetic acid (1 mL). To this reaction mixture, hydrazine hydrate (17.3 mg, 222 μmol) was added, and the mixture was allowed to stir for 12 hours at 70° C. Upon cooling to room temperature, the reaction mixture was quenched with aqueous sodium bicarbonate and mixed with DCM. The organic layer was separated, washed with brine, dried over magnesium sulfate, and concentrated in vacuo. The resulting solid was loaded onto silica gel and purified by column chromatography (silica gel, 24 g, 95% EtOAc in heptane) to give 1-benzyl-3-phenyl-6-(4H-1,2,4-triazol-3-yl)-1H-pyrazolo[3,4-d]pyrimidine (19.0 mg, 25.3%) as an yellow solid.

LCMS (ESI+): m/z 354.2 [M+H]⁺

Additional Compound Prepared in an Analogous Manner to Compound 282.

| | Exact | LCMS | |
|---|---|---|---|
| Compound | Mass | Found | Assignment |
| 182 | 329.1277 | 330.2 | [M + H]⁺ |

### Synthesis of Compound 284

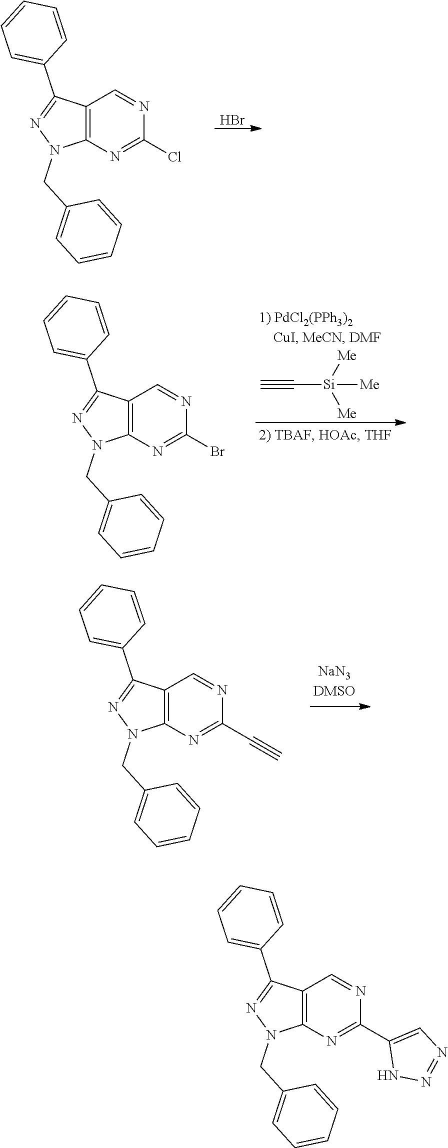

### 1-benzyl-6-bromo-3-phenyl-1H-pyrazolo[3,4-d]pyrimidine

A suspension of 1-benzyl-6-chloro-3-phenyl-1H-pyrazolo[3,4-d]pyrimidine (0.34 g, 1.05 mmol) in hydrogen bromide (concentrated in acetic acid, 3 mL) was stirred at 60° C. for 4 hours. Upon cooling to room temperature, the solvent was evaporated, and the mixture was diluted with DCM (10 mL) and quenched with saturated aqueous sodium bicarbonate. The layers were separated, and the aqueous layer was further extracted with DCM. The combined organic layers were dried over magnesium sulfate and evaporated to give a yellow solid. This material was purified by column chromatography (silica gel, 2% MeOH in DCM) to give 1-benzyl-6-bromo-3-phenyl-1H-pyrazolo[3,4-d]pyrimidine (340 mg, 89%) as a white solid.

### 1-benzyl-6-ethynyl-3-phenyl-1H-pyrazolo[3,4-d]pyrimidine

To a mixture of 1-benzyl-6-bromo-3-phenyl-1H-pyrazolo[3,4-d]pyrimidine (83 mg, 227 μmol), ethynyltrimethylsilane (33.3 mg, 340 μmol), palladium(II) bis(triphenylphosphane) dichloride (15.9 mg, 22.7 μmol), and iodocopper (0.0086 g, 45.3 μmol) in acetonitrile (2 mL) and DMF (1 mL) under argon was added triethylamine (229 mg, 2.27 mmol). The mixture was then stirred at 75° C. under argon for 15 hours. Upon cooling to room temperature, the mixture was diluted EtOAc (5 mL) and quenched with water (2 mL). The layers were separated, and the aqueous layer was further extracted with EtOAc. The combined organic layers were dried over magnesium sulfate and concentrated. The crude material was then dissolved in THF (1.5 mL) and TBAF (77.1 mg, 295 μmol) was added followed by acetic acid (14.9 mg, 249 μmol). The mixture was stirred at room temperature for 10 minutes. The mixture was diluted with DCM (5 mL) and quenched with 10% aqueous potassium carbonate (5 mL). The layers were separated, and the aqueous layer was further extracted with DCM. The combined organic layers were dried over magnesium sulfate and evaporated. The crude material was purified by silica gel column chromatography (15% EtOAc in heptane) to obtain 1-benzyl-6-ethynyl-3-phenyl-1H-pyrazolo[3,4-c]pyrimidine as a beige solid (40 mg, 57% yield).

LCMS (ESI+): m/z 311.1 [M+H]⁺

### 1-benzyl-3-phenyl-6-(1H-1,2,3-triazol-5-yl)-1H-pyrazolo[3,4-d]pyrimidine. Compound 284

A mixture of 1-benzyl-6-ethynyl-3-phenyl-1H-pyrazolo[3,4-d]pyrimidine (15 mg, 48.3 μmol) and sodium azide (3.76 mg, 57.9 μmol) in DMSO (0.5 mL) was heated in the microwave for 20 minutes at 150° C. Upon cooling to room temperature, the reaction was quenched with water (2 mL) and extracted with DCM. The organic layer was dried over magnesium sulfate and evaporated to obtain a brown solid. The solid was suspended in DCM and collected by vacuum filtration. The white solid cake was washed with DCM and dried under vacuum to obtain 1-benzyl-3-phenyl-6-(1H-1,2,3-triazol-5-yl)-1H-pyrazolo[3,4-d]pyrimidine as a white solid (5 mg, 35% yield).

LCMS (ESI+): m/z 354.2 [M+H]

### Synthesis of Compound 285.

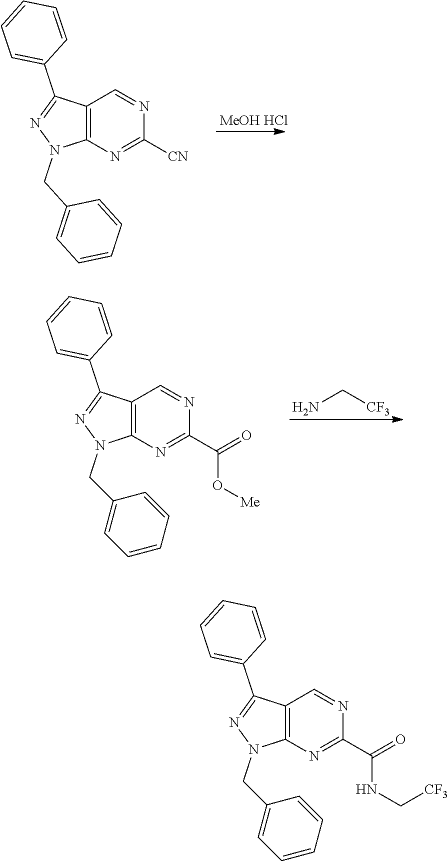

### methyl 1-benzyl-3-phenyl-1H-pyrazolo[3,4-d]pyrimidine-6-carboxylate

1-Benzyl-3-phenyl-1H-pyrazolo[3,4-d]pyrimidine-6-carbonitrile (410 mg, 1.31 mmol) was dissolved in hydrochloric acid in methanol (35 mL, 1.3 M HCl) and heated to 65° C. for 16 hours. Upon cooling to room temperature, the reaction was concentrated in vacuo and taken up in cold MeOH. The resulting precipitate was collected by vacuum filtration to give methyl 1-benzyl-3-phenyl-1H-pyrazolo[3,4-d]pyrimidine-6-carboxylate (266 mg, 58.9%) as a white solid.

LCMS (ESI+): m/z 345.2 [M+H]⁺

1-benzyl-3-phenyl-N-(2,2,2-trifluoroethyl)-1H-pyrazolo[3,4-d]pyrimidine-6-carboxamide. Compound 285

Methyl 1-benzyl-3-phenyl-1H-pyrazolo[3,4-d]pyrimidine-6-carboxylate (60 mg, 174 μmol), N,N-dimethylpyridin-4-amine (2.12 mg, 17.4 μmol), and 2,2,2-trifluoroethan-1-amine hydrochloride (353 mg, 2.61 mmol) were dissolved in THF (2 mL). Triethylamine (17.6 mg, 174 μmol) was slowly added, and the reaction mixture was heated to 75° C. for two days. Upon cooling to room temperature, the reaction mixture was diluted with water and EtOAc. The organic layer was separated, washed with brine, dried over magnesium sulfate, and concentrated in vacuo. The resulting solid was loaded onto silica gel and purified by column chromatography (silica gel, 24 g, 5% MeOH in DCM) to give 1-benzyl-3-phenyl-N-(2,2,2-trifluoroethyl)-1H-pyrazolo[3,4-d]pyrimidine-6-carboxamide (4.00 mg, 5.59%) as a white solid.

LCMS (ESI+): m/z 412.1 [M+H]⁺

### Synthesis of Compound 286

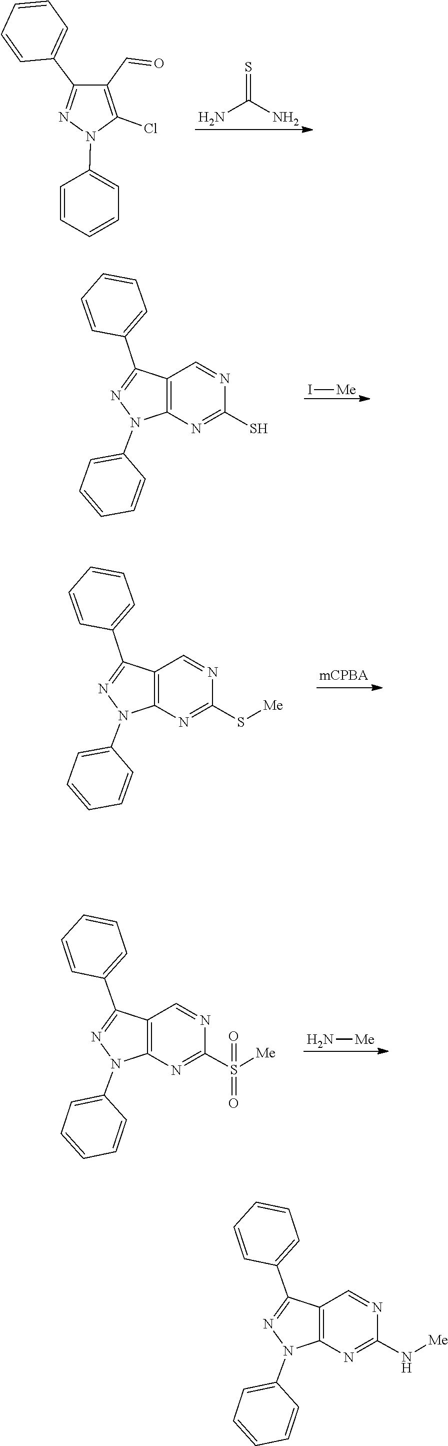

### 1,3-diphenyl-1H-pyrazolo[3,4-d]pyrimidine-6-thiol

A mixture of 5-chloro-1,3-diphenyl-1H-pyrazole-4-carbaldehyde (3.0 g, 10.6 mmol), sodium 2-methylpropan-2-olate (2.03 g, 21.2 mmol), and thiourea (1.21 g, 15.9 mmol) in ethanol (30 mL) was refluxed for 16 hours. After cooling to room temperature, 1 N aqueous HCl (25 mL) was added to the mixture. The formed precipitate was collected by vacuum filtration, and then the precipitate was suspended with methanol (20 mL) and ethyl acetate (40 mL). This precipitate was collected by vacuum filtration and dried under vacuum to give 1,3-diphenyl-1H-pyrazolo[3,4-d]pyrimidine-6-thiol (201 mg, 6.24%) as a yellow solid.

LCMS (ESI+): m/z 305.0 [M+H]⁺

### 6-(methylthio)-1,3-diphenyl-1H-pyrazolo[3,4-d]pyrimidine

To a solution of 1,3-diphenyl-1H-pyrazolo[3,4-d]pyrimidine-6-thiol (345 mg, 1.13 mmol) and sodium 2-methylpropan-2-olate (217 mg, 2.26 mmol) in DMF (8 mL) was added iodomethane (641 mg, 4.52 mmol) at room temperature. After stirring at room temperature for 2 hours, the mixture was diluted with water (50 mL), and the precipitate was collected by vacuum filtration to give 6-(methylthio)-1,3-diphenyl-1H-pyrazolo[3,4-d]pyrimidine (359 mg, 1.12 mmo) as a pale yellow solid. This crude product was used in the next reaction without further purification.

LCMS (ESI+): m/z 319.1 [M+H]⁺

### 6-(methylsulfonyl)-1,3-diphenyl-1H-pyrazolo[3,4-d]pyrimidine

To a suspension of crude 6-(methylthio)-1,3-diphenyl-1H-pyrazolo[3,4-d]pyrimidine (359 mg, 1.12 mmol) in DCM (10 mL) was added 3-chlorobenzene-1-carboperoxoic acid (751 mg, 3.36 mmol) at 0° C. After stirring at room temperature for 3 hours, additional 3-chlorobenzene-1-carboperoxoic acid (1.15 g, 6.72 mmol) was added, and the resulting mixture was stirred for 4 hours at room temperature. After cooling to 0° C., saturated aqueous sodium bicarbonate and saturated aqueous sodium thiosulfate (1/1, v/v, 100 mL) were added, and the mixture was stirred at 0° C. for 10 minutes. The mixture was extracted with DCM. The organic layer was washed with saturated aqueous sodium bicarbonate, dried over sodium sulfate, and concentrated under reduced pressure. The residue was resolidified in DCM to give 6-(methylsulfonyl)-1,3-diphenyl-1H-pyrazolo[3,4-d]pyrimidine (393 mg, 100% yield) as a pale yellow solid. Crude product was used in the next reaction without further purification.

LCMS (ESI+): m/z 351.0 [M+H]⁺

### N-methyl-1,3-diphenyl-1H-pyrazolo[3,4-d]pyrimidin-6-amine. Compound 286

6-Methanesulfonyl-1,3-diphenyl-1H-pyrazolo[3,4-d]pyrimidine (0.040 g, 0.114 mmol) and 2 M methylamine in THF (31.0 mg, 1 mmol) in THF (1 mL) was stirred for 16 hours at 50° C. After cooling to room temperature, the mixture was concentrated under reduced pressure. The residue was purified by column chromatography (silica gel, 24 g, 40% EtOAc in DCM) to give N-methyl-1,3-diphenyl-1H-pyrazolo[3,4-d]pyrimidin-6-amine (32.3 mg, 94.1%) as a colorless solid.

LCMS (ESI+): m/z 302.1 [M+H]⁺

### Synthesis of Compound 287.

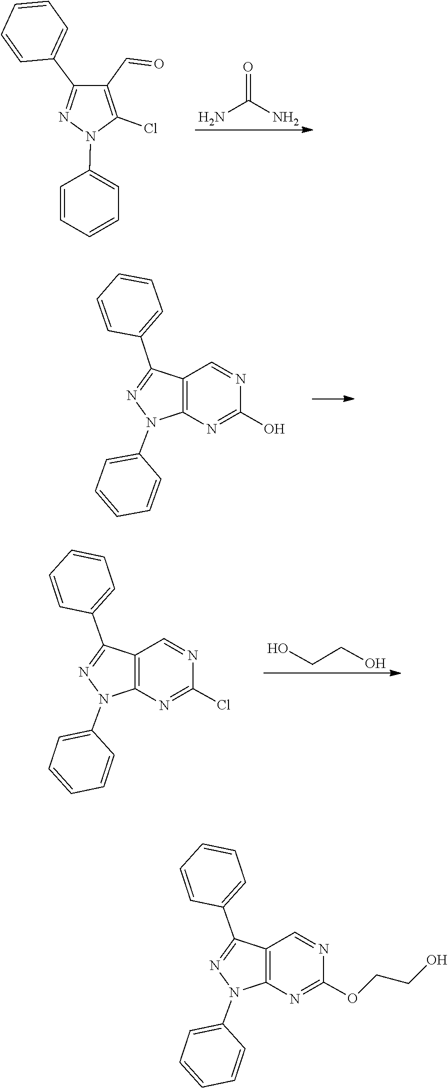

### 1,3-diphenyl-1H-pyrazolo[3,4-d]pyrimidin-6-ol

5-Chloro-1,3-diphenyl-1H-pyrazole-4-carbaldehyde (2.0 g, 7.07 mmol) was dissolved in 1-methylpyrrolidin-2-one (7 mL) and potassium carbonate (1.02 g, 7.42 mmol) and urea (445 mg, 7.42 mmol) were added. The mixture was then heated to 190° C. for one hour. Upon cooling to room temperature, the mixture was poured into water and a precipitate formed. The precipitate was collected by vacuum filtration and washed with water and ethanol. This process afforded 1,3-diphenyl-1H-pyrazolo[3,4-d]pyrimidin-6-ol (524 mg, 26%) as a beige solid.

LCMS (ESI+): m/z 289.1 [M+H]⁺

### 6-chloro-1,3-diphenyl-1H-pyrazolo[3,4-d]pyrimidine

In a flame dried vial, 1,3-diphenyl-1H-pyrazolo[3,4-d]pyrimidin-6-ol (1.8 g, 6.24 mmol) was dissolved in PCl₃ (13.8 mL). Two drops of DMF were added as a catalyst. The reaction was stirred at 100° C. for 16 hours. Upon cooling to room temperature, the reaction was quenched with water. The aqueous layer was extracted with DCM three times. The organic layers were combined, washed with brine, and dried over magnesium sulfate. The product was isolated via column chromatography (silica gel, 0-100% EtOAc in hexanes) to afford 6-chloro-1,3-diphenyl-1H-pyrazolo[3,4-d]pyrimidine (1.2 g, 63%).

LCMS (ESI+): m/z 307.1 [M+H]⁺

### 2-((1,3-diphenyl-1H-pyrazolo[3,4-d]pyrimidin-6-yl)oxy)ethan-1-ol. Compound 287

To a solution of ethane-1,2-diol (5.99 mg, 96.6 μmol) in DMF (1 mL) at 0° C. was added sodium hydride (3.85 mg, 87.9 μmol) and the mixture was stirred for 15 minutes at temperature. Then, 6-chloro-1,3-diphenyl-1H-pyrazolo[3,4-d]pyrimidine (45 mg, 146 μmol) was added, and the mixture was stirred for 2 hours while warming to room temperature. The reaction was quenched with ethanol and the solvent was evaporated. The crude residue was purified by silica gel column chromatography (12 g, 5% MeOH in DCM) to give 2-((1,3-diphenyl-1H-pyrazolo[3,4-d]pyrimidin-6-yl)oxy)ethan-1-ol (5 mg, 17%) as a white solid.

LCMS (ESI+): m/z 333.2 [M+H]⁺

### Synthesis of Compound 288.

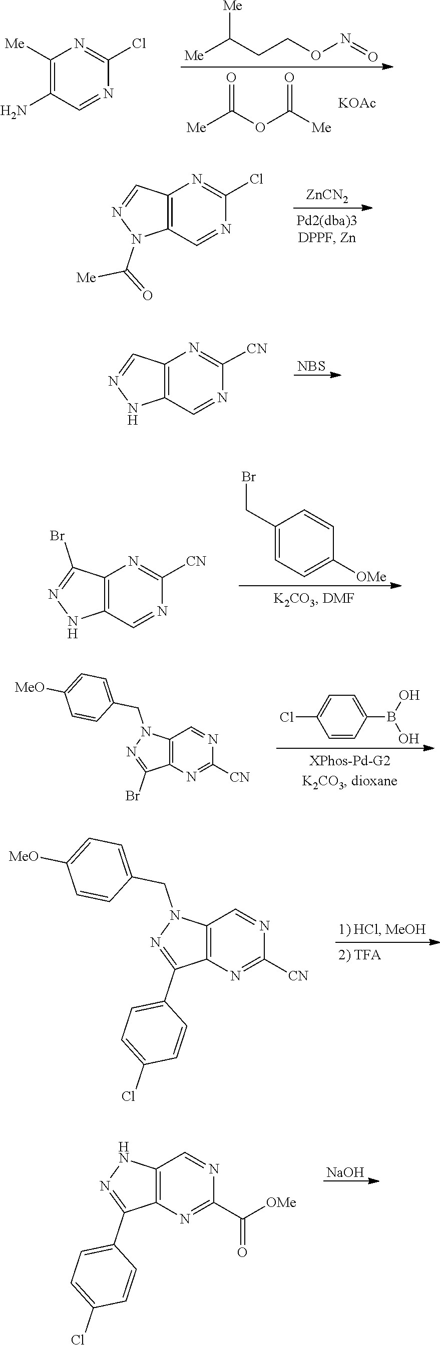

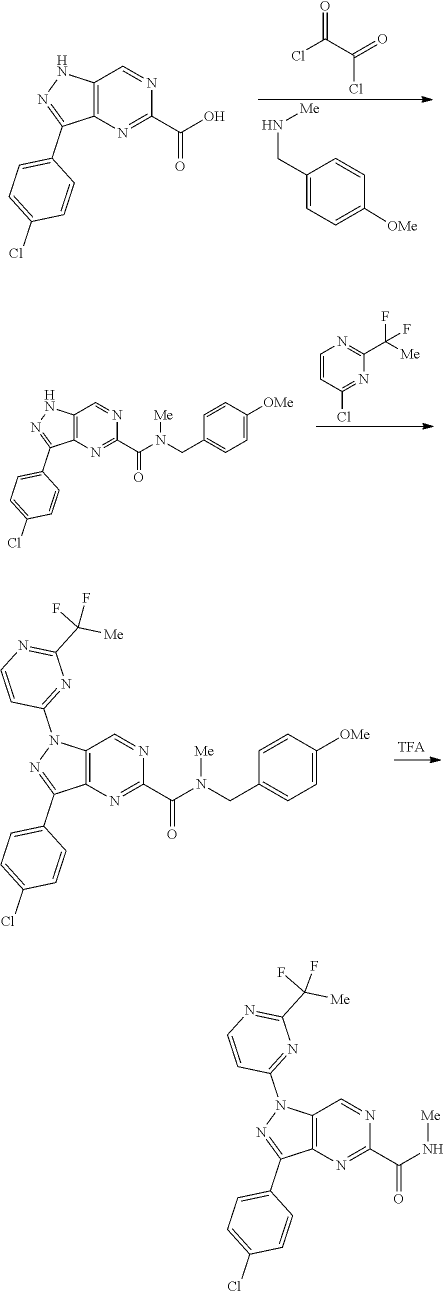

### 1-(5-chloro-1H-pyrazolo[4,3-d]pyrimidin-1-yl)ethan-1-one

A mixture of 2-chloro-4-methylpyrimidin-5-amine (8.3 g, 25 mmol), potassium acetate (3.7 g, 1.5 Eq, 38 mmol), acetic anhydride (10 g, 9.6 mL, 4 Eq, 0.10 mol), and AcOH (5 mL) in chloroform (50 mL) was refluxed for 2 hours. After cooling to room temperature, isopentyl nitrite (18 g, 21 mL, 6 Eq, 0.15 mol) was added and the mixture was refluxed for 3 hours. After cooling to room temperature, the mixture was diluted with EtOAc and filtered through short pad of silica gel. Upon concentration, the resulting sticky precipitate was triturated with MeOH and diluted with EtOAc, then filtered through silica gel. The filtrate was concentrated in vacuo. The residue was purified by column chromatography (silica gel, 80 g, 10% EtOAc in DCM). The resulting impure product was triturated with EtOAc in heptane. The resulting solid was collected, washed with heptane, and dried in vacuo to give desired product as a white solid. The mother liquor was concentrated in vacuo and the residue was purified by column chromatography (silica gel, 7 g, 100% DCM) to afford, when combined with the above material, 1-(5-chloro-1H-pyrazolo[4,3-d]pyrimidin-1-yl)ethan-1-one (1.92 g, 38%) as a white solid.

¹H NMR (400 MHz, CHLOROFORM-d) d ppm 9.74 (d, J=0.7 Hz, 1H), 8.35 (d, J=0.5 Hz, 1H), 2.85 (s, 3H).

LCMS (ESI+): m/z 197.0 [M+H]⁺

### 1H-pyrazolo[4,3-d]pyrimidine-5-carbonitrile

Under an argon atmosphere, a mixture of 1-(5-chloro-1H-pyrazolo[4,3-d]pyrimidin-1-yl)ethan-1-one (500 mg, 1 Eq, 2.54 mmol), dicyanozinc (329 mg, 1.1 Eq, 2.80 mmol), dppf (141 mg, 0.1 Eq, 254 μmol), zinc (33.3 mg, 0.2 Eq, 509 μmol), and Pd₂(dba)₃ (116 mg, 0.05 Eq, 127 μmol) in DMF (5 mL) was stirred at 100° C. for 1 hour. After cooling to room temperature, methylamine (2 M in THF, 2.5 mL, 2 Eq, 5.09 mmol) was added and the mixture was stirred for 1 hour. The mixture was poured into saturated aqueous NaHCO₃ and mixed with EtOAc. Insoluble solid was filtered off and the organic layer was separated. The aqueous mixture was extracted with EtOAc twice, then saturated with NaCl and further extracted with EtOAc. All organic extracts were combined, then washed with water and brine, dried over Na₂SO₄, and concentrated in vacuo. The residue was purified by column chromatography (silica gel, 25 g, 10-40% EtOAc in DCM) to afford 1H-pyrazolo[4,3-d]pyrimidine-5-carbonitrile (272 mg, 1.87 mmol, 73.7%) as a brown solid.

LCMS (ESI+): m/z 188.0 [M+H]⁺

### 3-bromo-1H-pyrazolo[4,3-d]pyrimidine-5-carbonitrile

To a solution of 1H-pyrazolo[4,3-d]pyrimidine-5-carbonitrile (180 mg, 1 Eq, 1.24 mmol) in DMF (6 mL) was added NBS (265 mg, 1.2 Eq, 1.49 mmol), and the mixture was stirred at room temperature for 50 minutes. The mixture was diluted with water, mixed with aqueous Na₂S₂O₃, and extracted with EtOAc. The extract was washed with water and brine, dried over Na₂SO₄, and concentrated in vacuo. The residue was purified by column chromatography (silica gel, 25 g, 0-20% EtOAc in DCM) to afford 3-bromo-1H-pyrazolo[4,3-d]pyrimidine-5-carbonitrile (188 mg, 839 μmol, 67.7%) as a pale yellow solid.

LCMS (ESI+): m/z 224.0 [M+H]⁺

### 3-bromo-1-(4-methoxybenzyl)-1H-pyrazolo[4,3-d]pyrimidine-5-carbonitrile

3-Bromo-1H-pyrazolo[4,3-d]pyrimidine-5-carbonitrile (984 mg, 4.39 mmol), DMF (15 mL), potassium carbonate (2 eq, 8.78 mmol), and 4-methoxybenzyl chloride (1.2 eq, 5.27 mmol) were combined and stirred at room temperature for 16 hours. After adding water to the mixture, the reaction was extracted with dichloromethane. The combined organic extracts were dried over sodium sulfate and concentrated under reduced pressure. The residue was then purified by silica gel column chromatography (0-10% EtOAc in DCM) to afford 3-bromo-1-[(4-methoxyphenyl)methyl]pyrazolo[4,3-d]pyrimidine-5-carbonitrile (727 mg, 2.11 mmol, 48.1%).

LCMS (ESI+): m/z 344.0 [M+H]⁺

### 3-(4-chlorophenyl)-1-[(4-methoxyphenyl)methyl]pyrazolo[4,3-d]pyrimidine-5-carbonitrile

3-Bromo-1-[(4-methoxyphenyl)methyl]pyrazolo[4,3-d]pyrimidine-5-carbonitrile (718 mg, 2.09 mmol), 4-chlorophenylboronic acid (1.5 eq, 3.13 mmol), chloro(2-dicyclohexylphosphino-2,4,6-triisopropyl-1,1-biphenyl)[2-(2-amino-1,1-biphenyl)]palladium(II) (0.1 eq, 0.209 mmol), dioxane (20 mL), and potassium carbonate (3 eq, 6.26 mmol) were combined and stirred at 80° C. under a nitrogen atmosphere for 16 hours. Upon cooling to room temperature, water was added to the mixture and it was extracted with dichloromethane. The combined organic extracts were dried over sodium sulfate and concentrated under reduced pressure. The residue was then purified by silica gel column chromatography (0-20% EtOAc in DCM) to afford 3-(4-chlorophenyl)-1-[(4-methoxyphenyl)methyl]pyrazolo[4,3-d]pyrimidine-5-carbonitrile (537 mg, 1.43 mmol, 68.4%).

LCMS (ESI+): m/z 376 [M+H]⁺

### methyl 3-(4-chlorophenyl)-1-[(4-methoxyphenyl)methyl]pyrazolo[4,3-d]pyrimidine-5-carboxylate

3-(4-Chlorophenyl)-1-[(4-methoxyphenyl)methyl]pyrazolo[4,3-d]pyrimidine-5-carbonitrile (526 mg, 1.40 mmol) was mixed with HCl in methanol (3 M, 5 mL) and dioxane (10 mL), and the reaction mixture was stirred at 70° C. for 16 hours. Upon cooling to room temperature, toluene was added to the mixture, and it was concentrated to give a crude solid. The solid was suspended in ethyl acetate, collected by vacuum filtration, and dried under vacuum to afford methyl 3-(4-chlorophenyl)-1-[(4-methoxyphenyl)methyl]pyrazolo[4,3-d]pyrimidine-5-carboxylate (554 mg, 1.36 mmol, 96.8%).

LCMS (ESI+): m/z 409 [M+H]⁺

### 3-(4-chlorophenyl)-1H-pyrazolo[4,3-d]pyrimidine-5-carboxylate

Methyl 3-(4-chlorophenyl)-1-[(4-methoxyphenyl)methyl]pyrazolo[4,3-d]pyrimidine-5-carboxylate (549 mg, 1.34 mmol) and trifluoroacetic acid (100 eq, 134 mmol) were stirred at 85° C. for 24 hours. Upon cooling to room temperature, toluene was added, and the mixture was concentrated to give a crude solid. The solid was suspended in ethyl acetate, collected by vacuum filtration, and dried under vacuum to afford methyl 3-(4-chlorophenyl)-1H-pyrazolo[4,3-d]pyrimidine-5-carboxylate (256 mg, 0.887 mmol, 66.0%).

LCMS (ESI+): m/z 289 [M+H]⁺

### 3-(4-chlorophenyl)-1H-pyrazolo[4,3-d]pyrimidine-5-carboxylic acid

Methyl 3-(4-chlorophenyl)-1H-pyrazolo[4,3-d]pyrimidine-5-carboxylate (250 mg, 0.865 mmol), tetrahydrofuran (2.5 mL), methanol (2.5 mL), and 1 M sodium hydroxide (aqueous, 1.5 eq, 1.30 mmol) were stirred at 60° C. for 6.5 hours. After cooling to room temperature, 1 M HCl in water (2.6 mL) was added. Ethyl acetate was then added, and the resulting precipitate was collected by vacuum filtration and dried under vacuum to afford 3-(4-chlorophenyl)-1H-pyrazolo[4,3-d]pyrimidine-5-carboxylic acid (189 mg, 0.688 mmol, 79.6%).

LCMS (ESI+): m/z 275 [M+H]⁺

### 3-(4-chlorophenyl)-N-[(4-methoxyphenyl)methyl]-N-methyl-1H-pyrazolo[4,3-d]pyrimidine-5-carboxamide

3-(4-Chlorophenyl)-1H-pyrazolo[4,3-d]pyrimidine-5-carboxylic acid (189 mg, 0.688 mmol), dichloromethane (3.5 mL), oxalyl chloride (1.5 eq, 1.03 mmol), and 1 drop of DMF were stirred at room temperature for 1 hour. Toluene was added, and the reaction mixture was concentrated to dryness. Dichloromethane (7 mL), triethylamine (5 eq, 3.44 mmol), and 4-methoxy-N-methylbenzylamine (1.5 eq, 1.03 mmol) were added, and the mixture was stirred for 3 hours at room temperature. At this time, additional 4-methoxy-N-methylbenzylamine (1.5 eq, 1.03 mmol) and triethylamine (5 eq, 3.44 mmol) were added, and the reaction mixture was stirred for 16 hours. Water was added, and the mixture was extracted with dichloromethane. The organic layer was dried over sodium sulfate and concentrated under reduced pressure. The residue was purified by silica gel column chromatography (0-50% EtOAc in DCM) to afford 3-(4-chlorophenyl)-N-[(4-methoxyphenyl)methyl]-N-methyl-1H-pyrazolo[4,3-d]pyrimidine-5-carboxamide (166 mg, 0.408 mmol, 59.3%).

LCMS (ESI+): m/z 408 [M+H]⁺

### 3-(4-chlorophenyl)-1-[2-(1,1-difluoroethyl)pyrimidin-4-yl]-N-[(4-methoxyphenyl)methyl]-N-methyl-pyrazolo[4,3-d]pyrimidine-5-carboxamide

3-(4-Chlorophenyl)-N-[(4-methoxyphenyl)methyl]-N-methyl-1H-pyrazolo[4,3-d]pyrimidine-5-carboxamide (158 mg, 0.388 mmol) and 4-chloro-2-(1,1-difluoroethyl)pyrimidine (1.5 eq, 0.582 mmol) were added to a mixture of DMF (4 mL) and potassium carbonate (2 eq, 0.776 mmol). The mixture was then heated at 40° C. for 24 hours. Water was added, and the mixture was extracted with dichloromethane. The organic layer was then dried over sodium sulfate and concentrated under reduced pressure. The residue was then purified by silica gel column chromatography (0-10% EtOAc in DCM) to afford 3-(4-chlorophenyl)-1-[2-(1,1-difluoroethyl)pyrimidin-4-yl]-N-[(4-methoxyphenyl)methyl]-N-methyl-pyrazolo[4,3-d]pyrimidine-5-carboxamide (49.2 mg, 0.0895 mmol, 23.0%).

LCMS (ESI+): m/z 550 [M+H]⁺

### 3-(4-chlorophenyl)-1-[2-(1,1-difluoroethyl)pyrimidin-4-yl]-N-methyl-pyrazolo[4,3-d]pyrimidine-5-carboxamide. Compound 288

3-(4-Chlorophenyl)-1-[2-(1,1-difluoroethyl)pyrimidin-4-yl]-N-[(4-methoxyphenyl)methyl]-N-methyl-pyrazolo[4,3-d]pyrimidine-5-carboxamide (46.7 mg, 0.0849 mmol) and trifluoroacetic acid (100 eq, 8.49 mmol) were heated to 80° C. for 2 hours. Upon cooling to room temperature, toluene was added, and the mixture was concentrated under reduced pressure. Ethyl acetate was added, and the resulting precipitate was collected by vacuum filtration to afford 3-(4-chlorophenyl)-1-[2-(1,1-difluoroethyl)pyrimidin-4-yl]-N-methyl-pyrazolo[4,3-d]pyrimidine-5-carboxamide (17.9 mg, 0.0416 mmol, 49.0%).

LCMS (ESI+): m/z 430 [M+H]⁺

Synthesis of Compound 289.

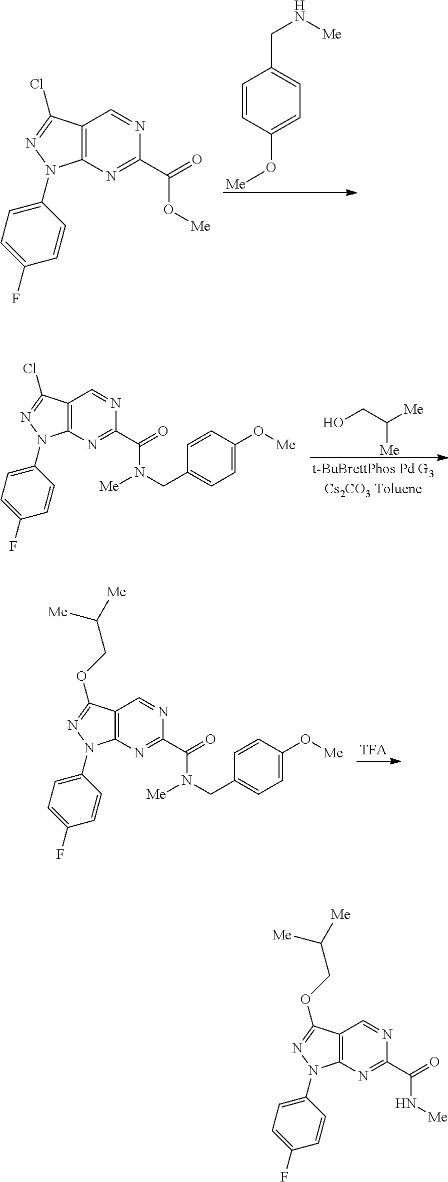 [SMILES: COC(=O)c1ncc2c(Cl)nn(-c3ccc(F)cc3)c2n1 ; CNCc1ccc(OC)cc1 ; COc1ccc(CN(C)C(=O)c2ncc3c(Cl)nn(-c4ccc(F)cc4)c3n2)cc1 ; CC(C)CO ; COc1ccc(CN(C)C(=O)c2ncc3c(OCC(C)C)nn(-c4ccc(F)cc4)c3n2)cc1 ; CNC(=O)c1ncc2c(OCC(C)C)nn(-c3ccc(F)cc3)c2n1]

### 3-chloro-1-(4-fluorophenyl)-N-(4-methoxybenzyl)-N-methyl-1H-pyrazolo[3,4-d]pyrimidine-6-carboxamide

A solution of methyl 3-chloro-1-(4-fluorophenyl)-1H-pyrazolo[3,4-d]pyrimidine-6-carboxylate (70 mg, 228 μmol) in [(4-methoxyphenyl)methyl](methyl)amine (0.3 mL) was stirred at 85° C. for 7 hours. Upon cooling to room temperature, the material was adsorbed onto silica and purified by column chromatography (silica gel, 0-60% EtOAc in heptane) to afford 3-chloro-1-(4-fluorophenyl)-N-[(4-methoxyphenyl)methyl]-N-methyl-1H-pyrazolo[3,4-d]pyrimidine-6-carboxamide (80 mg, 82%).

LCMS (ESI+): m/z 426 [M+H]⁺

### 1-(4-fluorophenyl)-3-isobutoxy-N-(4-methoxybenzyl)-N-methyl-1H-pyrazolo[3,4-d]pyrimidine-6-carboxamide

To a flame dried vial were added 3-chloro-1-(4-fluorophenyl)-N-[(4-methoxyphenyl)methyl]-N-methyl-1H-pyrazolo[3,4-d]pyrimidine-6-carboxamide (0.050 g, 117 μmol), 9-[di-tert-butyl({3,6-dimethoxy-2-[2,4,6-tris(propan-2-yl)phenyl]phenyl})-λ⁵-phosphanyl]-8-aza-9 palladatricyclo[8.4.0.0^{2,7}]tetradeca-1(14),2(7),3,5,10,12-hexaen-9-yl methanesulfonate (14.9 mg, 17.5 μmol), and cesium carbonate (76.2 mg, 234 μmol). Then, under argon, 2-methylpropan-1-ol (17.3 mg, 234 μmol) and toluene (1 mL) were added, and the mixture was stirred at 90° C. for 1 hour. The reaction was cooled to room temperature, and the mixture was diluted with EtOAc (5 mL). The crude mixture was then filtered through celite and evaporated to dryness. This material was then adsorbed onto silica and purified by column chromatography (silica gel, 0-60% EtOAc in heptane) to provide 1-(4-fluorophenyl)-3-isobutoxy-N-(4-methoxybenzyl)-N-methyl-1H-pyrazolo[3,4-d]pyrimidine-6-carboxamide (22 mg, 40.5%).

LCMS (ESI+): m/z 464.1 [M+H]⁺

### 1-(4-fluorophenyl)-3-isobutoxy-N-methyl-1H-pyrazolo[3,4-d]pyrimidine-6-carboxamide. Compound 188

A solution of 1-(4-fluorophenyl)-N-[(4-methoxyphenyl)methyl]-N-methyl-3-(2-methylpropoxy)-1H-pyrazolo[3,4-d]pyrimidine-6-carboxamide (22 mg, 47.4 μmol) in trifluoroacetic acid (1 mL) was stirred at 85° C. for 30 minutes. Upon cooling to room temperature, the solvent was evaporated, and the crude material was adsorbed onto silica gel. This material was then purified by column chromatography (silica gel, 0-5% MeOH in DCM) to afford 1-(4-fluorophenyl)-3-isobutoxy-N-methyl-1H-pyrazolo[3,4-d]pyrimidine-6-carboxamide (10 mg, 61.7%).

LCMS (ESI+): m/z 344.1 [M+H]⁺

### Synthesis of Compound 290.

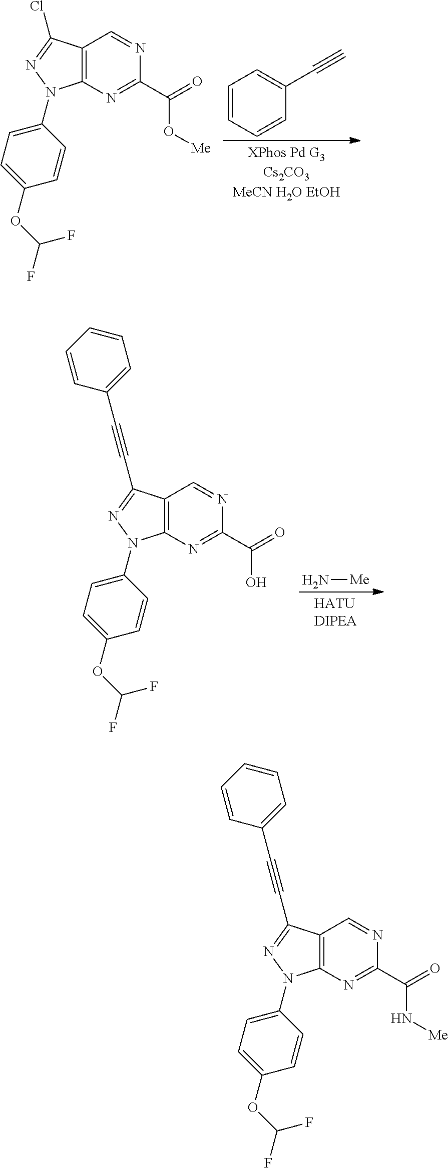

### methyl 3-chloro-1-(4-(difluoromethoxy)phenyl)-1H-pyrazolo[3,4-d]pyrimidine-6-carboxylate

Prepared according to the procedure described for 3-chloro-1-(4-fluorophenyl)-N-methyl-1H-pyrazolo[3,4-d]pyrimidine-6-carboxamide.

¹H NMR (400 MHz, CHLOROFORM-d) δ ppm 9.36 (1H, s), 8.24-8.30 (2H, m), 7.33 (2H, d, J=8.8 Hz), 6.37-6.79 (1H, m), 4.12 (3H, s).

### 1-(4-(difluoromethoxy)phenyl)-3-(phenylethynyl)-1H-pyrazolo[3,4-d]pyrimidine-6-carboxylic acid

To a suspension of methyl 3-chloro-1-(4-(difluoromethoxy)phenyl)-1H-pyrazolo[3,4-d]pyrimidine-6-carboxylate (5.00 g, 1 Eq, 14.1 mmol), phenylacetylene (4.32 g, 4.6 mL, 3 Eq, 42.3 mmol), and cesium carbonate (13.8 g, 3 Eq, 42.3 mmol) in MeCN (70 mL) was added Xphos Pd G3 (1.19 g, 0.1 Eq, 1.41 mmol) at room temperature under nitrogen atmosphere. After degassing under, the mixture was stirred for 6 hours at 90° C. The reaction was cooled to room temperature and mixed with ethanol (6 mL) and water (3 mL). This mixture was then stirred at room temperature for 16 hours. The mixture was extracted with EtOAc twice. The organic layers were concentrated until the volume of solvent became to 1/3. Then, the mixture was diluted with EtOAc/Heptane(1:1). The resulting precipitate was collected by vacuum filtration to afford 1-(4-(difluoromethoxy)phenyl)-3-(phenylethynyl)-1H-pyrazolo[3,4-d]pyrimidine-6-carboxylic acid (1.65 g, 4.06 mmol, 28.8%) as a dark brown solid. The obtained material was used for next reaction without further purification.

### 1-(4-(difluoromethoxy)phenyl)-N-methyl-3-(phenylethynyl)-1H-pyrazolo[3,4-d]pyrimidine-6-carboxamide. Compound 290

To a solution of 1-(4-(difluoromethoxy)phenyl)-3-(phenylethynyl)-1H-pyrazolo[3,4-d]pyrimidine-6-carboxylic acid (1.14 g, 1 Eq, 2.81 mmol), DIPEA (725 mg, 0.98 mL, 2 Eq, 5.61 mmol), and HATU (2.13 g, 2 Eq, 5.61 mmol) in DMF (15 mL) was added methylamine (436 mg, 2 mL, 8 molar, 5 Eq, 14.0 mmol) at 0° C. After stirring for 1 hour at room temperature, the reaction mixture was diluted with EtOAc and acidified with 1 N HCl (aq.). The layers were separated, and the organic layer was and washed with water and brine. The organic layer was dried over magnesium sulfate, filtered, and concentrated. The resulting residue was purified with silica gel column chromatography (25-80% EtOAc in heptane) to give 1-(4-(difluoromethoxy)phenyl)-N-methyl-3-(phenylethynyl)-1H-pyrazolo[3,4-d]pyrimidine-6-carboxamide (460 mg, 1.10 mmol, 39.1%) as a pale-yellow solid.

¹H NMR: (400 MHz, CHLOROFORM-d) d ppm 9.42 (s, 1H), 8.26-8.35 (m, 2H), 8.02-8.16 (m, 1H), 7.65-7.74 (m, 2H), 7.40-7.51 (m, 3H), 7.31-7.39 (m, 2H), 6.59 (t, J=73.60 Hz, 1H), 3.15 (d, J=5.14 Hz, 3H).

LCMS (ESI+): m/z 420.1 [M+H]⁺

### Synthesis of Compound 291.

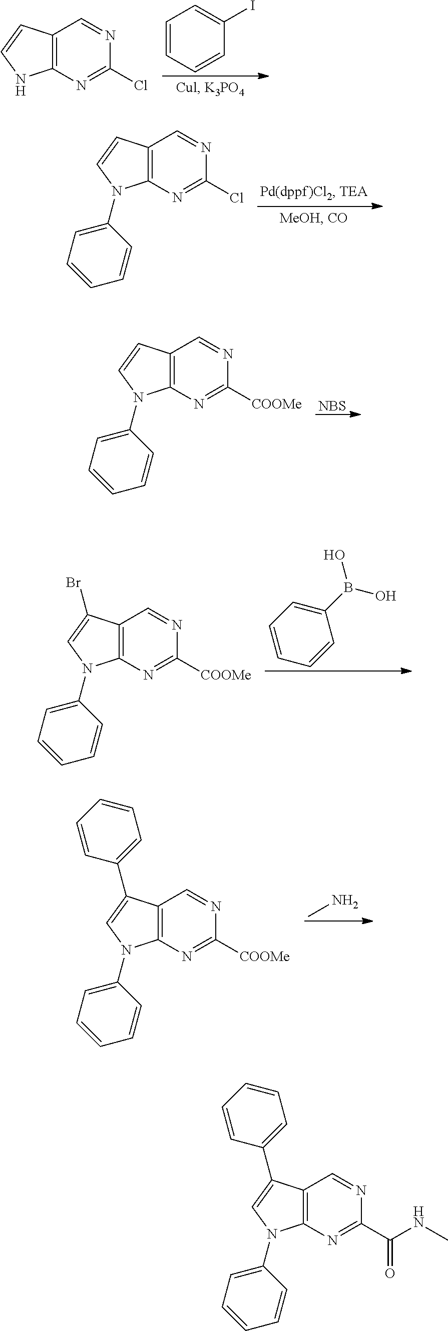

### 2-chloro-7-phenyl-7H-pyrrolo[2,3-d]pyrimidine

A mixture of 2-chloro-7H-pyrrolo[2,3-d]pyrimidine (2.0 g, 13 mmol), iodobenzene (2.6 g, 13 mmol), cyclohexane-1,2-diamine (1.2 g, 10.6 mmol), CuI (0.9 g, 5.3 mmol), and K₃PO₄ (5.5 g, 26 mmol) in dioxane (20 mL) was stirred at 100° C. under nitrogen for 16 hours. Upon cooling, the mixture was filtered to remove undissolved solids, and the filtrate was concentrated in vacuo. The resulting residue was purified by column chromatography (silica gel, 20% EtOAc in petroleum ether) to give 2-chloro-7-phenyl-7H-pyrrolo[2,3-d]pyrimidine (1.86 g, 62% yield) as a white solid.

LCMS: r.t=1.90 mins, (M+H)⁺=230, purity: 90% (@214 nm)

### methyl 7-phenyl-7H-pyrrolo[2,3-d]pyrimidine-2-carboxylate

A mixture of 2-chloro-7-phenyl-7H-pyrrolo[2,3-d]pyrimidine (1.7 g, 7.4 mmol), Pd(dppf)Cl₂ (0.54 g, 0.74 mmol), and TEA (1.5 g, 14.8 mmol) in MeOH (100 mL) was stirred at 100° C. under CO (30 bar) for 24 hours. Then, the mixture was concentrated under reduced pressure, and the crude was purified by column chromatography (silica gel, 9% MeOH in DCM) to give methyl 7-phenyl-7H-pyrrolo[2,3-d]pyrimidine-2-carboxylate (1.7 g, 89% yield) as an orange solid.

LCMS: r.t=1.63 mins, (M+H)⁺=254, purity: 90% (@214 nm)

### methyl 5-bromo-7-phenyl-7H-pyrrolo[2,3-d]pyrimidine-2-carboxylate

To a mixture of methyl 7-phenyl-7H-pyrrolo[2,3-d]pyrimidine-2-carboxylate (0.4 g, 1.58 mmol) in MeCN (10 mL) was added NBS (0.42 g, 2.37 mmol) slowly at room temperature. The reaction mixture was then stirred at room temperature for 2 hours. The mixture was concentrated, and the resulting residue was purified by column chromatography (silica gel, 50% EtOAc in petroleum ether) to give methyl 5-bromo-7-phenyl-7H-pyrrolo[2,3-d]pyrimidine-2-carboxylate (0.5 g, 95% yield) as a light-yellow solid.

LCMS: r.t=1.30 mins, (M+H)⁺=332, purity: 92% (@214 nm)

### methyl 5,7-diphenyl-7H-pyrrolo[2,3-d]pyrimidine-2-carboxylate

A mixture of methyl 5-bromo-7-phenyl-7H-pyrrolo[2,3-d]pyrimidine-2-carboxylate (200 mg, 0.6 mmol), phenylboronic acid (110 mg, 0.9 mmol), Pd(PPh₃)₄ (70 mg, 0.06 mmol), and K₂CO₃ (248 mg, 1.8 mmol) in dioxane (4 mL) and water (0.4 mL) was stirred at 100° C. under nitrogen for 2 hours. Upon cooling to room temperature, the mixture was concentrated under reduced pressure, and the residue was purified by column chromatography (silica gel, 65% EtOAc in petroleum ether) to give methyl 5,7-diphenyl-7H-pyrrolo[2,3-d]pyrimidine-2-carboxylate (30 mg, 15% yield) as a light-yellow solid.

LCMS: r.t=1.96 mins, (M+H)⁺=330, purity: 98% (@214 nm)

### N-methyl-5,7-diphenyl-7H-pyrrolo[2,3-d]pyrimidine-2-carboxamide. Compound 190

A mixture of methyl 5,7-diphenyl-7H-pyrrolo[2,3-d]pyrimidine-2-carboxylate (30 mg, 0.09 mmol) in methylamine (33% solution in ethanol, 3 mL) was stirred at 45° C. for 3 hours. The mixture was concentrated under reduced pressure, and the crude was purified by preparative HPLC (C18 column, 0-75% MeCN in water) to give N-methyl-5,7-diphenyl-7H-pyrrolo[2,3-d]pyrimidine-2-carboxamide (15 mg, 50% yield) as a white solid.

LCMS: r.t=1.86 mins, (M+H)⁺=329.0, purity: 95% (@220 nm)

¹H NMR (400 MHz, DMSO-d6) δ ppm 9.52 (s, 1H), 8.81 (d, J=4.7 Hz, 1H), 8.62 (s, 1H), 8.02 (d, J=7.5 Hz, 2H), 7.94 (d, J=7.1 Hz, 2H), 7.65 (t, J=7.9 Hz, 2H), 7.51 (dt, J=19.4, 7.5 Hz, 3H), 7.40 (d, J=7.4 Hz, 1H), 2.86 (d, J=4.8 Hz, 3H).

### Synthesis of Compound 3.

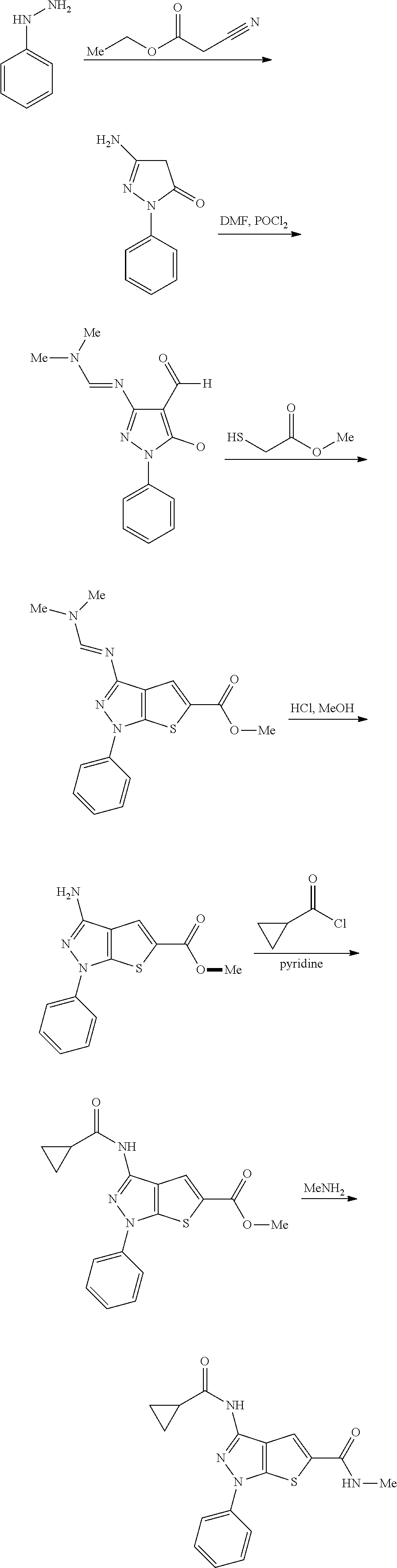

### 5-amino-2-phenyl-2,4-dihydro-3H-pyrazol-3-one

Ethyl cyanoacetate (295 mL, 1 eq.) was added to sodium ethoxide (21% in ethanol, 2.07 L, 2 equiv) at room temperature. The reaction mixture was stirred for 30 minutes, and phenyl hydrazine (273 mL, 2.77 mol) was added. The reaction mixture was heated at 80° C. for 20 hours. The ethanol was distilled off and water (4 L) was added to dissolve the resulting solid. The aqueous solution was extracted with ether (3×2 L) to remove impurities. Finally, acetic acid (318 mL, 2 equiv) was added to the aqueous solution, and the resulting solid was collected by vacuum filtration. The solid was then washed with water and dried on high vacuum for two days to give 5-amino-2-phenyl-2,4-dihydro-3H-pyrazol-3-one as an orange solid (300 g, 61.7%).

¹H NMR: (300 MHz, DMSO-d6) δ 7.83 (dt, J=8.1, 1.1 Hz, 2H), 7.48-7.22 (m, 2H), 7.17-6.94 (m, 1H), 6.42 (s, 2H), 3.58 (s, 2H).

### N′-(5-chloro-4-formyl-1-phenyl-1H-pyrazol-3-yl)-N,N-dimethylformimidamide

To DMF (156 mL, 3 equiv) in an ice/water bath was slowly added POCl₃ (431.6 mL, 7 equiv). The mixture was stirred for 15 minutes in the ice bath. Then, 5-amino-2-phenyl-2,4-dihydro-pyrazol-3-one (118 g, 0.674 mol) was added. The mixture was heated to 110° C. for 2 hours. The mixture was then poured into an aqueous solution of K₂CO₃ (991.8 g, 10.65 equiv in 4 L of water). The resulting solution was extracted with EtOAc, washed with brine, dried over sodium sulfate, filtered, and the solvent evaporated. The residue was taken up in hexanes and a solid formed. This solid was collected by vacuum filtration and dried under vacuum to give N′-(5-chloro-4-formyl-1-phenyl-1H-pyrazol-3-yl)-N,N-dimethylformimidamide (178 g, 95.5%).

¹H NMR: (600 MHz, Acetone-d6) δ 10.03 (s, 1H), 8.27 (s, 1H), 7.65 (d, J=7.8 Hz, 2H), 7.60 (t, J=7.8 Hz, 2H), 7.53 (t, J=7.4 Hz, 1H), 3.16 (s, 3H), 3.08 (s, 3H).

HPLC: RT: 4.30 min (93.7%) (Zorbac C18, 95% to 0% Water (0.1% TFA) in CH₃CN for 10 min, flow 1.5 mL/min)

### methyl-3-(((dimethylamino)methylene)amino)-1-phenyl-1H-thieno[2,3-c]pyrazole-5-carboxylate

A mixture of N′-(5-chloro-4-formyl-1-phenyl-1H-pyrazol-3-yl)-N,N-dimethyl-formamidine (178 g, 0.643 mol), methyl thioglycolate (61.8 mL, 1.02 equiv), and potassium carbonate (187 g, 2.1 equiv) were refluxed for 16 hours in dioxane (1.85 L). The reaction mixture was cooled down to room temperature, and the resulting solution was filtered through a pad of celite. The resulting filtrate was evaporated in vacuo. The resulting solid was suspended in 4 L of hexanes with 5% EtOAc overnight. The formed precipitate was collected by vacuum filtration and washed with 1 L of hexanes to provide methyl-3-(((dimethylamino)methylene)amino)-1-phenyl-1H-thieno[2,3-c]pyrazole-5-carboxylate (188 g, 89% yield) as a yellow solid.

¹H NMR: (600 MHz, Acetone-d6) δ 8.41 (s, 1H), 7.90 (s, 1H), 7.77 (d, J=7.7 Hz, 2H),

7.59 (dd, J=8.4, 7.6 Hz, 2H), 7.29 (t, J=7.4 Hz, 1H), 3.91 (s, 3H), 3.20 (s, 3H), 3.11 (s, 3H).

HPLC: RT: 7.11 min (90.8%) (Zorbac C18, 95% to 0% Water (0.1% ammonium acetate) in CH3CN for 10 min, flow 1.5 mL/min)

### methyl 3-amino-1-phenyl-1H-thieno[2,3-c]pyrazole-5-carboxylate

A mixture of methyl (E)-3-(((dimethylamino)methylene)amino)-1-phenyl-1H-thieno[2,3-c]pyrazole-5-carboxylate (188 g, 0.573 mol) and HCl (1.16 L of a 1 M solution in methanol, 2 equiv) was refluxed for 16 hours in methanol (3.8 L). The mixture was cooled to room temperature and poured into ice water (8 L). The resulting precipitate was collected by vacuum filtration, washed with water, and dried for 2 days under vacuum. The resulting solid was crushed and vacuum dried for another 3 days giving methyl 3-amino-1-phenyl-1H-thieno[2,3-c]pyrazole-5-carboxylate (155 g, 99% yield).

¹H NMR: (600 MHz, Acetone-d6) δ 7.92 (s, 1H), 7.67 (d, J=7.9 Hz, 2H), 7.56 (t, J=7.9 Hz, 2H), 7.24 (t, J=7.4 Hz, 1H), 5.54 (d, J=12.1 Hz, 1H), 3.90 (s, 3H).

HPLC: RT: 7.02 min (98%) (Zorbac C18, 95% to 0% Water (0.1% TFA) in CH₃CN for 10 min, flow 1.5 mL/min)

### Methyl 3-(cyclopropanecarboxamido)-1-phenyl-1H-thieno[2,3-c]pyrazole-5-carboxylate

Methyl 3-amino-1-phenyl-1H-thieno[2,3-c]pyrazole-5-carboxylate (152 g, 0.554 mol) was dissolved in pyridine (627 mL) and cooled to 0° C. Then, cyclopropanecarbonyl chloride (63.5 mL, 1.2 eq) was added slowly over 20 minutes. After stirring for 3 hours at room temperature, 0.1 more equivalents were added of the acid chloride (10 mL) and the reaction mixture was stirred for another hour. The reaction mixture was quenched with 6 L of water. The resulting solid was filtered and washed with 4 L of water followed by 4 L of hexanes. The solid was swished in 5 L of hexanes for 16 hours. The solid was collected by vacuum filtration and dried in vacuo to provide methyl 3-(cyclopropanecarboxamido)-1-phenyl-1H-thieno[2,3-c]pyrazole-5-carboxylate (186 g, 99% yield). The material was used as is for the last step.

¹H NMR: (600 MHz, DMSO-d6) δ 11.49 (s, 1H), 8.17 (s, 1H), 7.72 (d, J=7.8 Hz, 2H), 7.63 (t, J=8.0 Hz, 2H), 7.38 (dt, J=14.7, 6.6 Hz, 1H), 3.87 (s, 3H), 2.00 (d, J=4.3 Hz, 1H), 0.96-0.76 (m, 4H).

HPLC: RT: 7.89 min (96%) (Zorbac C18, 95% to 0% Water (0.1% TFA) in CH3CN for 10 min, flow 1.5 mL/min)

### 3-(cyclopropanecarboxamido)-N-methyl-1-phenyl-1H-thieno[2,3-c]pyrazole-5-carboxamide. Compound 3

Methyl 3-(cyclopropanecarboxamido)-1-phenyl-1H-thieno[2,3-c]pyrazole-5-carboxylate (186 g, 0.543 mmol) was dissolved in methylamine (33% by weight in ethanol, 3.56 L, 32.6 mol). The reaction mixture was stirred for 16 hours during which time a precipitate formed. The resulting slurry was then heated at 70° C. for 3 hours under vacuum to remove excess methylamine. At this time, almost all the methylamine was removed leaving approximately 2 L of ethanol. The resulting suspension was cooled to 40° C. and 4 L of hexanes was added. The resulting solid was collected by vacuum filtration and washed with 4 L of hexanes. The material was then dried under vacuum for 48 hours to provide 3-(cyclopropanecarboxamido)-N-methyl-1-phenyl-1H-thieno[2,3-c]pyrazole-5-carboxamide (159 g, 86% yield) as an off-white solid.

¹H NMR: (600 MHz, DMSO-d6) δ 11.39 (s, 1H), 8.74 (d, J=4.5 Hz, 1H), 8.11 (s, 1H), 7.72 (d, J=7.7 Hz, 2H), 7.61 (t, J=8.0 Hz, 2H), 7.34 (t, J=7.4 Hz, 1H), 2.77 (d, J=4.5 Hz, 3H), 2.06-1.89 (m, 1H), 0.98-0.70 (m, 4H).

LCMS (ESI+): m/z 341.0 [M+H]⁺

### Synthesis of Compound 38.

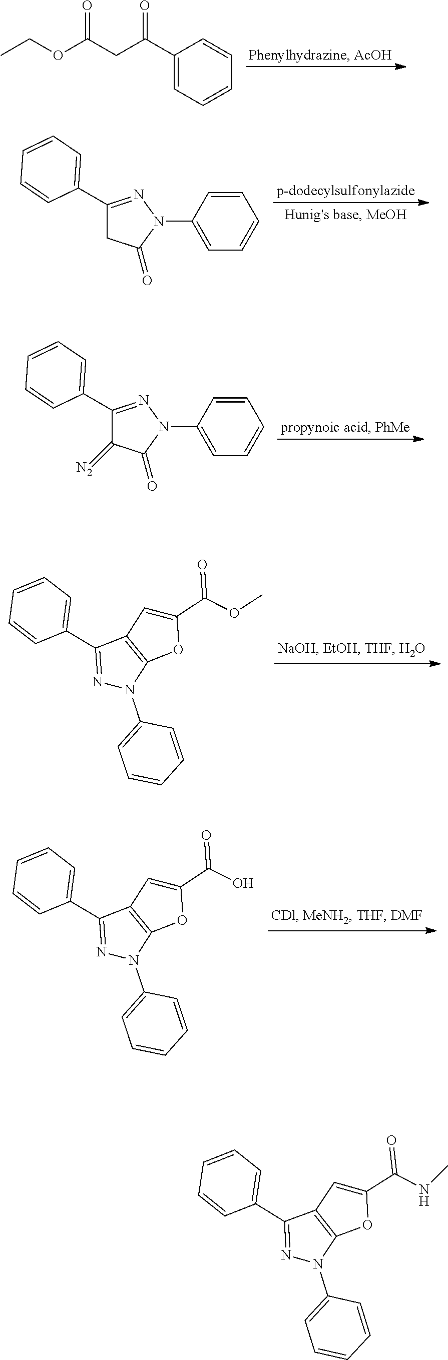

### 1,3-diphenyl-1H-pyrazol-5(4H)-one

Ethyl 3-oxo-3-phenylpropanoate (3.84 g, 20 mmol) and phenylhydrazine (2.16 g, 20.0 mmol) were mixed with acetic acid (15 mL), and the mixture was heated to reflux for 4 hours. After cooling to room temperature, the crude mixture was poured into water (100 mL) and stirred to form a precipitate. The resulting precipitate was collected by filtration and dissolved in methylene chloride. This solution was dried over Na₂SO₄ and then concentrated under reduced pressure. The resulting residue was recrystallized from methylene chloride/heptane to give 1,3-diphenyl-1H-pyrazol-5(4H)-one (3.45 g, 73.0%) as a solid.

¹H NMR: (400 MHz, CDCl₃) δ 7.99 (dd, J=8.8, 1.2 Hz, 2H), 7.80-7.78 (m, 2H), 7.48-7.42 (m, 4H), 7.27-7.23 (m, 2H), 3.86 (s, 2H).

### 4-diazo-1,3-diphenyl-1H-pyrazol-5(4H)-one

To a solution of 1,3-diphenyl-1H-pyrazol-5(4H)-one (472 mg, 2 mmol) and N-ethyl-N-isopropylpropan-2-amine (775 mg, 6.00 mmol) in methanol (10 mL) was added 4-dodecylbenzenesulfonyl azide (0.773 g, 2.19 mmol) at 0° C. After stirring for 3 hours at temperature, the reaction was warmed to room temperature and toluene (10 mL) was added. The solvent was then removed under reduced pressure. The resulting residue was purified by column chromatography (silica gel column, 20% EtOAc in heptane) to give 4-diazo-1,3-diphenyl-1H-pyrazol-5(4H)-one (205 mg, 39%) as a solid.

¹H NMR: (400 MHz, CDCl₃) δ 8.02-8.00 (m, 2H), 7.72-7.68 (m, 2H), 7.52-7.44 (m, 4H), 7.28-7.21 (m, 2H).

### methyl 1,3-diphenyl-1H-furo[2,3-c]pyrazole-5-carboxylate

A mixture of 4-diazo-1,3-diphenyl-1H-pyrazol-5(4H)-one (95 mg, 0.3622 mmol), methyl propiolate (76.1 mg, 0.9055 mmol), and toluene (2 mL) was heated at 150° C. for 2 days in a sealed tube. After cooling to room temperature, the resulting mixture was concentrated under reduced pressure. The residue was purified by column chromatography (silica gel column, 20% EtOAc in heptane) to give methyl 1,3-diphenyl-1H-furo[2,3-c]pyrazole-5-carboxylate (43.7 mg, 38%) as a solid.

¹H NMR: (400 MHz, CDCl₃) δ 8.05-7.99 (m, 4H), 7.62 (s, 1H), 7.55-7.47 (m, 4H), 7.43-7.41 (m, 1H), 7.30-7.26 (m, 1H), 3.98 (s, 3H).

### 1,3-diphenyl-1H-furo[2,3-c]pyrazole-5-carboxylic acid

Methyl 1,3-diphenyl-1H-furo[2,3-c]pyrazole-5-carboxylate (44 mg, 138 μmol) was dissolved in ethanol (6 mL), water (2 mL), and THF (2 mL) and sodium hydroxide (319 mg, 7.97 mmol) was added. The reaction was heated to reflux for 2 hours. The crude reaction mixture was diluted with water and acidified with 1M HCl to pH 2.0. At this time, a white precipitate formed which was then collected by filtration. The filtrate was suspended in DCM (5 mL) and minimal methanol was added until the mixture became homogeneous. The solution was dried over magnesium sulfate and concentrated to afford 1,3-diphenyl-1H-furo[2,3-c]pyrazole-5-carboxylic acid (assumed 42 mg, 100%) which was used directly without further purification or characterization.

### N-methyl-1,3-diphenyl-1H-furo[2,3-c]pyrazole-5-carboxamide. Compound 38

1,3-Diphenyl-1H-furo[2,3-c]pyrazole-5-carboxylic acid (42 mg, 138 μmol) was dissolved in THF (1.4 mL) and N,N-dimethylformamide (460 μL) and 1-(1H-imidazole-1-carbonyl)-1H-imidazole (89.5 mg, 552 μmol) was added under argon. The reaction was stirred at 60° C. for one hour. At this time, methylamine (2 M in ethanol, 414 μL, 828 μmol) was added in one portion and the reaction stirred at 60° C. for an additional hour. The reaction mixture was diluted with ethyl acetate and washed with water, sodium bicarbonate (aqueous), and brine. The organic layer was dried over magnesium sulfate and concentrated under reduced pressure. The crude material was purified by column chromatography (20 g silica gel column, 5% MeOH in DCM) to afford N-methyl-1,3-diphenyl-1H-furo[2,3-c]pyrazole-5-carboxamide (35.0 mg, 80%) as a solid.

¹H NMR: (400 MHz, DMSO-d6) δ 8.69 (q, J=4.4 Hz, 1H), 8.11 (dd, J=9.2 Hz, 7.2 Hz, 4H), 7.87 (s, 1H), 7.68 (t, J=7.6 Hz, 2H), 7.56 (t, J=7.6 Hz, 2H), 7.48 (t, J=7.2 Hz, 1H), 7.42 (t, J=7.4 Hz, 1H), 2.89 (d, mv=4.8 Hz, 3H). LCMS (ESI+): m/z 318.2 [M+H]⁺ HPLC: RT: 2.62 min

Additional Compounds Prepared in an Analogous Manner to Compound 38:

| | Exact | LCMS | |
|---|---|---|---|
| Compound | Mass | Found | Assignment |
| 45 | 320.1353 | 321.2 | [M + H]⁺ |
| 292 | 331.1321 | 336.1 | [M + H]⁺ |
| 28 | 351.0775 | 352.1 | [M + H]⁺ |

### Synthesis of Compound 34.

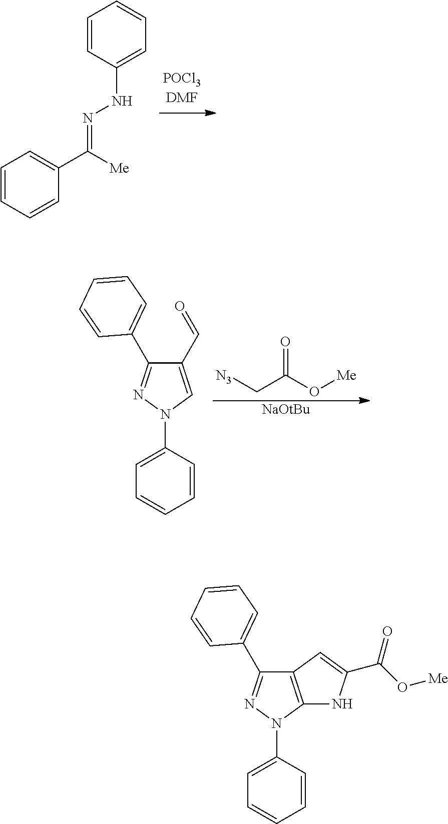

### 1,3-diphenyl-1H-pyrazole-4-carbaldehyde

To DMF (2.35 g, 32.2 mmol) was added phosphoryl trichloride (1.64 g, 10.7 mmol) at 0° C., and the mixture was stirred at 0° C. for 30 minutes. To the resulting mixture was added (E)-1-phenyl-2-(1-phenylethylidene)hydrazine (1.13 g, 5.37 mmol) in DMF (2 mL), and the mixture was stirred at 0° C. for 1 hour. The mixture was then heated to 60° C. for 2 hours. After cooling to room temperature, the mixture was neutralized with saturated aqueous sodium bicarbonate and diluted with water. The mixture was then extracted with ethyl acetate, and the organic layer was washed with water and dried over sodium sulfate. The solution was concentrated under reduced pressure to give 1,3-diphenyl-1H-pyrazole-4-carbaldehyde (1.23 g, 92.4%) as a pale yellow solid.

¹H NMR (400 MHz, CDCl₃) d ppm 10.06 (s, 1H), 8.55 (s, 1H), 7.84-7.78 (m, 4H), 7.50-7.40 (m, 6H).

### methyl 1,3-diphenyl-1,6-dihydropyrrolo[2,3-c]pyrazole-5-carboxylate. Compound 34

To a suspension of 1,3-diphenyl-1H-pyrazole-4-carbaldehyde (1.23 g, 4.95 mmol) in methanol (20 mL) was added sodium 2-methylpropan-2-olate (1.90 g, 19.8 mmol), and the mixture was stirred at room temperature for 10 minutes. After cooling to 0° C., methyl 2-azidoacetate (2.27 g, 19.8 mmol) was added, and the mixture was stirred at 0° C. for 30 minutes. The cold bath was removed, and the mixture was stirred at room temperature for an additional 2.5 hours. Water (40 mL) was added, and a precipitate formed. The solid was collected by vacuum filtration. The obtained solid was suspended with toluene (40 mL), and the mixture was heated at reflux for 2 hours. After cooling to room temperature, the mixture was dried over sodium sulfate and concentrated to a volume of 15 mL. The mixture was diluted with hexanes (50 mL) and a precipitate formed. This material was collected by vacuum filtration to give methyl 1,3-diphenyl-1,6-dihydropyrrolo[2,3-c]pyrazole-5-carboxylate (609 mg, 38.7%) as a brown solid.

LCMS (ESI+): m/z 318.0 [M+H]⁺

### Synthesis of Compound 31.

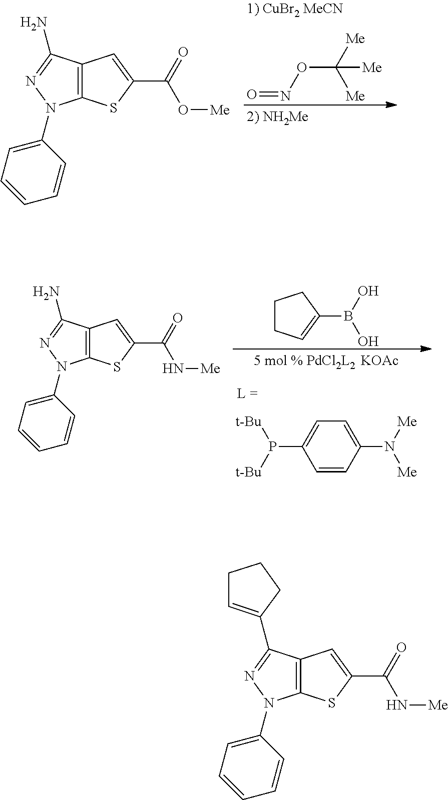

### 3-bromo-N-methyl-1-phenyl-1H-thieno[2,3-c]pyrazole-5-carboxamide

In a flame dried flask, copper(II) bromide (498 mg, 2.23 mmol) was dissolved in acetonitrile (22.3 mL) under an argon atmosphere. Then, tert-butyl nitrite (229 mg, 2.23 mmol) was added, and the solution was cooled to 0° C. 3-Amino-N-methyl-1-phenyl-1H-thieno[2,3-c]pyrazole-5-carboxamide (0.61 g, 2.23 mmol) was then suspended in acetonitrile (22.3 mL) and added slowly to the cold reaction. The solution was then left to return to room temperature and stirred for 1 hour. The reaction was quenched with 1M NaOH and extracted with ethyl acetate 3 times. The organic phases were combined, dried over magnesium sulfate, and concentrated. The ester was then isolated via flash column chromatography (silica gel, 0-100% EtOAc in hexanes). The intermediate product was dissolved in methanolic methyl amine (33%, 20 mL), and stirred for 12 hours at room temperature. At this time, the mixture was evaporated to dryness to afford 3-bromo-N-methyl-1-phenyl-1H-thieno[2,3-c]pyrazole-5-carboxamide (190 mg, 25%).

LCMS (ESI+): m/z 336.0 [M+H]⁺

### 3-(cyclopent-1-en-1-yl)-N-methyl-1-phenyl-1H-thieno[2,3-c]pyrazole-5-carboxamide. Compound 31

3-Bromo-N-methyl-1-phenyl-1H-thieno[2,3-c]pyrazole-5-carboxamide (75 mg, 223 μmol), palladium(II) bis(4-(di-tert-butylphosphanyl)-N,N-dimethylaniline) dichloride (7.84 mg, 11.1 μmol), cyclopent-1-en-1-ylboronic acid (49.9 mg, 446 μmol), and potassium acetate (65.6 mg, 669 μmol) were heated to 90° C. in butan-1-ol (2 mL) for 16 hours. Upon cooling to room temperature, the reaction was diluted with dichloromethane (5 mL) and washed with water (3×5 mL). The organic phase was concentrated, and the residue was purified by silica gel column chromatography (0-100% EtOAc in heptane) to afford 3-(cyclopent-1-en-1-yl)-N-methyl-1-phenyl-1H-thieno[2,3-c]pyrazole-5-carboxamide (51 mg, 71%).

LCMS (ESI+): m/z 324 [M+H]⁺

¹H NMR (400 MHz, DMSO-d6) d ppm 8.64 (d, 1H, J=4.4 Hz), 8.21 (s, 1H), 7.81 (d, 2H, J=7.6 Hz), 7.65 (t, 2H, J=7.6 Hz), 7.39 (t, 1H, J=7.6 Hz), 6.57 (s, 1H), 2.90-2.85 (m, 5H), 2.66-2.63 (m, 2H), 2.07 (p, 2H, J=7.2 Hz).

### Synthesis of Compound 49.

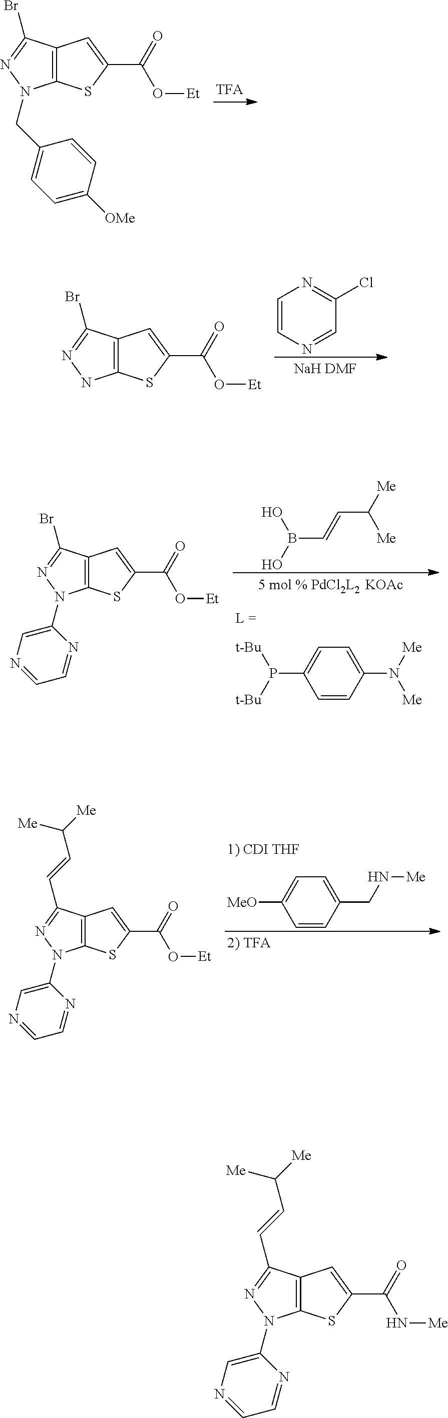

### 3-bromo-1H-thieno[2,3-c]pyrazole-5-carboxylate

In a reaction vial, ethyl 3-bromo-1-[(4-methoxyphenyl)methyl]-1H-thieno[2,3-c]pyrazole-5-carboxylate (WO2010111060 A1, 0.45 g, 1.13 mmol) was dissolved in trifluoroacetic acid (20 mL) and the reaction was stirred at 60° C. for 1 hour. The solvent was evaporated, and the residue was purified by column chromatography (silica gel, 0-5% MeOH in DCM) to provide ethyl 3-bromo-1H-thieno[2,3-c]pyrazole-5-carboxylate (294 mg, 95%).

LCMS (ESI+): m/z 275 [M+H]⁺

¹H NMR (400 MHz, DMSO-d6) d ppm 7.73 (s, 1H), 4.35 (q, 2H, J=7 Hz), 1.30 (t, 3H, J=7 Hz).

### ethyl 3-bromo-1-(pyrazin-2-yl)-1H-thieno[2,3-c]pyrazole-5-carboxylate

In a flame dried vial, ethyl 3-bromo-1H-thieno[2,3-c]pyrazole-5-carboxylate (90 mg, 327 μmol) was dissolved in DMF (1.1 mL) under an argon atmosphere. The reaction was then cooled to 0° C. before sodium hydride (15.6 mg, 392 μmol) was added in portions. The hydrogen was allowed to evolve at 0° C. Once hydrogen evolution was complete, 2-chloropyrazine (186 mg, 1.63 mmol) was added to the reaction. The solution was then transferred to a flame dried microwave vial under argon atmosphere. The solution was then heated in the microwave at 100° C. for 2 hours. The reaction was quenched with water and a precipitate formed. This solid was collected by vacuum filtration to give ethyl 3-bromo-1-(pyrazin-2-yl)-1H-thieno[2,3-c]pyrazole-5-carboxylate (68 mg, 59%).

LCMS (ESI+): m/z 353.0 [M+H]⁺

### ethyl (E)-3-(3-methylbut-1-en-1-yl)-1-(pyrazin-2-yl)-1H-thieno[2,3-c]pyrazole-5-carboxylate

Prepared via Suzuki coupling as described for 3-(cyclopent-1-en-1-yl)-N-methyl-1-phenyl-1H-thieno[2,3-c]pyrazole-5-carboxamide.

### (E)-N-methyl-3-(3-methylbut-1-en-1-yl)-1-(pyrazin-2-yl)-1H-thieno[2,3-c]pyrazole-5-carboxamide. Compound 49

Ethyl 3-[(1E)-3-methylbut-1-en-1-yl]-1-(pyrazin-2-yl)-1H-thieno[2,3-c]pyrazole-5-carboxylate (80 mg, 233 μmol) was dissolved in dioxane (3 mL) and 1-(1H-imidazole-1-carbonyl)-1H-imidazole (151 mg, 932 μmol) was added. The reaction mixture was then stirred at 60° C. for one hour. [(4-Methoxyphenyl)methyl](methyl)amine (151 mg, 1.00 mmol) was then added and the mixture was stirred for an additional 16 hours overnight. The reaction was quenched with water, and extracted with ethyl acetate. The organic solution was dried over sodium sulfate and concentrated under reduced pressure. To this residue was added trifluoroacetic acid (5 mL), and the mixture was stirred at 90° C. for 2 hours. Upon cooling to room temperature, the solvent was removed under reduced pressure, and the crude was purified on silica gel column chromatography (0-100% EtOAc in hexanes) to provide (E)-N-methyl-3-(3-methylbut-1-en-1-yl)-1-(pyrazin-2-yl)-1H-thieno[2,3-c]pyrazole-5-carboxamide (36 mg, 47%).

LCMS (ESI+): m/z 328.1 [M+H]⁺

### Synthesis of Compound 32.

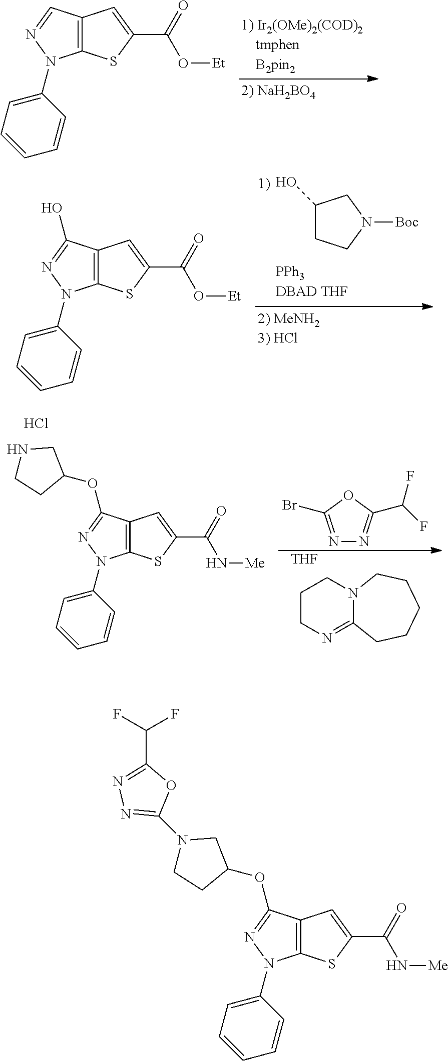

### ethyl 3-hydroxy-1-phenyl-1H-thieno[2,3-c]pyrazole-5-carboxylate

3,4,7,8-Tetramethyl-1,10-phenanthroline (34.5 mg, 146 μmol), bis((1Z,5Z)-cycloocta-1,5-diene); dimethyl-2,4-dioxa-1,3-diiridabicyclo[1.1.0]butane-2,4-diium-1,3-diuide (48.6 mg, 73.4 μmol), and 4,4,5,5-tetramethyl-2-(tetramethyl-1,3,2-dioxaborolan-2-yl)-1,3,2-dioxaborolane (977 mg, 3.85 mmol) were heated to 100° C. in dioxane (9.2 mL) under argon for one hour. Then, ethyl 1-phenyl-1H-thieno[2,3-c]pyrazole-5-carboxylate (1 g, 3.67 mmol) was added, and the reaction mixture was stirred for 16 hours at 100° C. Upon cooling to room temperature, the reaction was concentrated under reduced pressure to give crude ethyl 1-phenyl-3-(tetramethyl-1,3,2-dioxaborolan-2-yl)-1H-thieno[2,3-c]pyrazole-5-carboxylate. To the crude ethyl 1-phenyl-3-(tetramethyl-1,3,2-dioxaborolan-2-yl)-1H-thieno[2,3-c]pyrazole-5-carboxylate was added sodium perborate hydrate (745 mg, 7.32 mmol), and the material was stirred in a mixture of THF (29.2 mL) and water (29.2 mL) for 1 hour at room temperature. The reaction was quenched with aqueous ammonium chloride and extracted with ethyl acetate. The combined organic extracts were dried over sodium sulfate and concentrated under reduced pressure. The solid was taken up in DCM and a precipitate formed. The white precipitate was collected by vacuum filtration and washed with an excess of dichloromethane to afford ethyl 3-hydroxy-1-phenyl-1H-thieno[2,3-c]pyrazole-5-carboxylate (700 mg, 67%).

LCMS (ESI+): m/z 289.1 [M+H]⁺

### N-methyl-1-phenyl-3-[(3R)-pyrrolidin-3-yloxy]-1H-thieno[2,3-c]pyrazole-5-carboxamide hydrochloride

In a flame dried vial, ethyl 3-hydroxy-1-phenyl-1H-thieno[2,3-c]pyrazole-5-carboxylate (0.5 g, 1.82 mmol), tert-butyl (3S)-3-hydroxypyrrolidine-1-carboxylate (509 mg, 2.72 mmol), di-tert-butyl azodicarboxylate (1.04 g, 4.55 mmol), and triphenylphosphine (595 mg, 2.27 mmol) were dissolved in THF (18.1 mL) under an argon atmosphere. The reaction was stirred at room temperature for 3.5 hours. The reaction was quenched with water and extracted 3 times with EtOAc. The organic phases were combined, washed with brine, dried over magnesium sulfate, and concentrated. The ester compound was then isolated via flash silica gel chromatography (40% EtOAc in heptane). This intermediate was then dissolved in 3 mL of methyl amine (33% in EtOH) and heated for 3 hours at 80° C. The solvent was evaporated, and the boc group was removed by dissolving in methanolic HCl (3 M, 5 mL) and stirring for 1 hour at room temperature. The solvent was removed in vacuo to afford N-methyl-1-phenyl-3-[(3R)-pyrrolidin-3-yloxy]-1H-thieno[2,3-c]pyrazole-5-carboxamide hydrochloride (623 mg, 100%).

LCMS (ESI+): m/z 343.0 [M+H]⁺

### 3-{[(3R)-1-[5-(difluoromethyl)-1,3,4-oxadiazol-2-yl]pyrrolidin-3-yl]oxy}-N-methyl-1-phenyl-1H-thieno[2,3-c]pyrazole-5-carboxamide. Compound 32

In a flame dried vial, N-methyl-1-phenyl-3-[(3R)-pyrrolidin-3-yloxy]-1H-thieno[2,3-c]pyrazole-5-carboxamide hydrochloride (70 mg, 184 μmol) and 2-bromo-5-(difluoromethyl)-1,3,4-oxadiazole (47.5 mg, 239 μmol) was dissolved in THF (1 mL) under an argon atmosphere. Then, 2H,3H,4H,6H,7H,8H,9H,10H-pyrimido[1,2-a]azepine (36.3 mg, 239 μmol) was added to the solution. The reaction was left to stir at room temperature for 2.5 hours. The reaction was quenched with aqueous ammonium chloride and extracted 3 times with DCM. The organic phases were combined, dried over magnesium sulfate, and concentrated. The crude material was then purified by preparative HPLC (C18 column, 10-100% MeCN in water with 0.1% TFA) to provide 3-{[(3R)-1-[5-(difluoromethyl)-1,3,4-oxadiazol-2-yl]pyrrolidin-3-yl]oxy}-N-methyl-1-phenyl-1H-thieno[2,3-c]pyrazole-5-carboxamide (68 mg, 80% yield).

LCMS (ESI+): m/z 460.5 [M+H]⁺

### Example 5

### Synthesis of Compound 295.

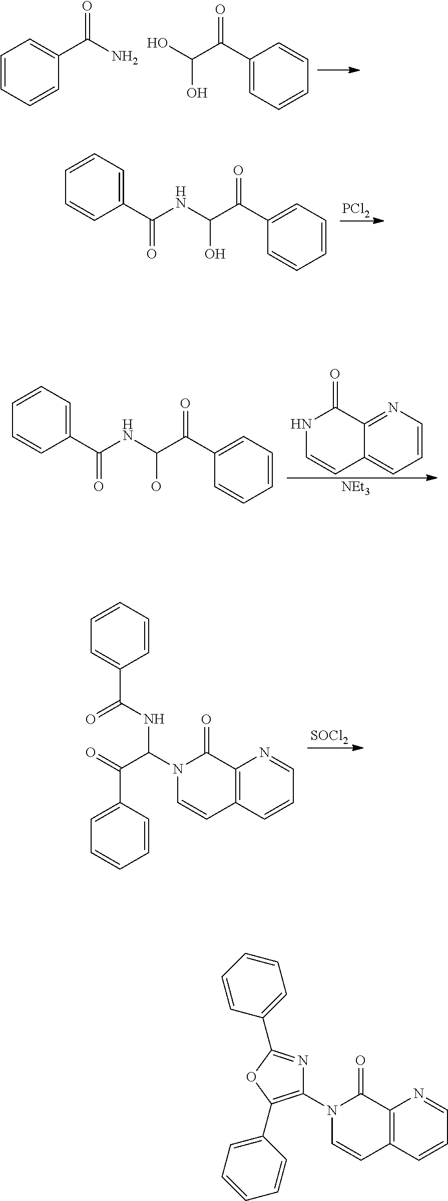

### N-(1-hydroxy-2-oxo-2-phenylethyl)benzamide

A mixture of benzamide (5.0 g, 41.3 mmol) and 2,2-dihydroxy-1-phenylethan-1-one (8.16 g, 1.3 equiv) in 1,4-dioxane (100 mL) was heated to 90° C. overnight. After 15 hours, the reaction was cooled to room temperature and concentrated to dryness. The resulting paste was triturated with water (50 mL), filtered, washed with water, and dried under vacuo to give N-(1-hydroxy-2-oxo-2-phenylethyl)benzamide (7.97 g, 31.2 mmol, 75.6%) as light yellow solid.

LCMS (ESI⁺): m/z 278.1 [M+Na]⁺

### N-(chloro(phenyl)methyl)benzamide

To a solution of N-(1-hydroxy-2-oxo-2-phenylethyl)benzamide (200 mg, 1 Eq, 0.88 mmol) in DCM (5 mL) was added phosphorous pentachloride (183 mg, 1 Eq, 0.88 mmol) and the reaction was heated to 50° C. for 2 hours. The reaction was then cooled down, and the solvent was slowly evaporated. The resulting orange solid was then dried for 2 hours and the resulting N-(chloro(phenyl)methyl)benzamide was used directly in the next step.

### N-(2-oxo-1-(8-oxo-1,7-naphthyridin-7(8H)-yl)-2-phenylethyl)benzamide

N-(Chloro(phenyl)methyl)benzamide was dissolved in THF (5 ml) and triethylamine (89.1 mg, 1 Eq, 880 μmol) was added followed by isoquinolin-1(2H)-one (128 mg, 1 Eq, 880 μmol). After 1 hour at room temperature, the reaction was completed at which point water (10 mL) was added and a precipitate began to form. The precipitate was filtered off and provided N-(2-oxo-1-(8-oxo-1,7-naphthyridin-7(8H)-yl)-2-phenylethyl)benzamide (102 mg, 266 μmol, 61.8%) as a pure white solid.

### 7-(2,5-diphenyloxazol-4-yl)-1,7-naphthyridin-8(7H)-one. Compound 295

N-(2-Oxo-1-(8-oxo-1,7-naphthyridin-7(8H)-yl)-2-phenylethyl)benzamide (264 mg, 1 Eq, 0.69 mmol) was added to a 40 mL vial to which thionyl chloride (1.63 g, 1.0 mL) was added and brought to 65° C. for 15 hours. After the reaction was completed, the reaction was concentrated very slowly by rotary evaporation to remove thionyl chloride. Once concentrated, 7-(2,5-diphenyloxazol-4-yl)-1,7-naphthyridin-8(7H)-one (75 mg, 0.21 mmol, 30%) was triturated out with diethyl ether and dried in vacuo.

LCMS (ESI+): m/z 366.2 [M+H]⁺.

1H NMR (400 MHz, DMSO-d6) δ ppm 8.90 (d, J=3.42 Hz, 1H), 8.35 (d, J=8.07 Hz, 1H), 8.15-8.17 (m, 2H), 7.88 (dd, J=8.19, 4.52 Hz, 1H), 7.73 (d, J=7.34 Hz, 1H), 7.63-7.56 (m, 5H), 7.50-7.39 (m, 3H), 6.90 (d, J=7.34 Hz, 1H).

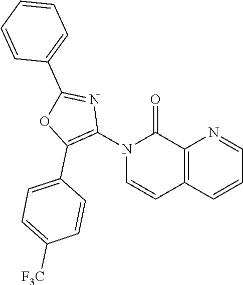 [SMILES: O=c1c2ncccc2ccn1-c1nc(-c2ccccc2)oc1-c1ccc(C(F)(F)F)cc1]

### 7-(2-phenyl-5-(4-(trifluoromethyl)phenyl)oxazol-4-yl)-1,7-naphthyridin-8(7H)-one. Compound 296

Compound 296 was prepared in a manner analogous to compound 295, starting from benzamide and 2,2-dihydroxy-1-(4-trifluoromethylphenyl)-ethan-1-one. Scale on last step 400 mg (0.887 mmol), 300 mg 78% yield.

LCMS (ESI+): m/z 433.9 [M+H]⁺.

¹H NMR (300 MHz, DMSO-d₆) δ 8.89 (dd, J=4.4, 1.7 Hz, 1H), 8.28 (dd, J=8.2, 1.7 Hz, 1H), 8.23-8.12 (m, 2H), 7.90-7.76 (m, 5H), 7.72 (d, J=7.4 Hz, 1H), 7.69-7.57 (m, 3H), 6.89 (d, J=7.4 Hz, 1H).

Additional Compounds were Prepared in a Similar Manner to Compound 295 & 296:

| | Exact mass | Observed | LCMS |
|---|---|---|---|
| Compound | (g/mol) | m/z (+) | Assignment |
| 297 | 379.1321 | 379.94 | [M + H]⁺ |
| 298 | 380.1273 | 380.93 | [M + H]⁺ |
| 299 | 383.107 | 383.98 | [M + H]⁺ |
| 300 | 385.0885 | 385.67 | [M + H]⁺ |
| 301 | 389.0567 | 389.84 | [M + H]⁺ |
| 302 | 395.127 | 395.91 | [M + H]⁺ |
| 303 | 395.127 | 395.93 | [M + H]⁺ |
| 304 | 395.127 | 395.94 | [M + H]⁺ |
| 305 | 397.1038 | 398.0 | [M + H]⁺ |
| 306 | 399.0775 | 399.91 | [M + H]⁺ |
| 307 | 399.0775 | 400.0 | [M + H]⁺ |
| 308 | 399.0775 | 400.0 | [M + H]⁺ |
| 309 | 400.0727 | 401.04 | [M + H]⁺ |
| 310 | 401.0976 | 401.67 | [M + H]⁺ |
| 311 | 401.0976 | 402.0 | [M + H]⁺ |
| 312 | 405.1113 | 405.86 | [M + H]⁺ |
| 313 | 405.1113 | 405.87 | [M + H]⁺ |
| 314 | 406.143 | 407.4 | [M + H]⁺ |
| 315 | 407.0473 | 408.07 | [M + H]⁺ |
| 316 | 408.1586 | 408.98 | [M + H]⁺ |
| 317 | 413.0931 | 414.1 | [M + H]⁺ |
| 318 | 415.1132 | 416.0 | [M + H]⁺ |
| 319 | 417.068 | 417.64 | [M + H]⁺ |
| 320 | 417.068 | 418.0 | [M + H]⁺ |
| 321 | 417.0947 | 418.0 | [M + H]⁺ |
| 322 | 419.0882 | 419.96 | [M + H]⁺ |
| 323 | 419.0882 | 419.91 | [M + H]⁺ |
| 324 | 427.1445 | 428.21 | [M + H]⁺ |
| 325 | 431.1081 | 432.0 | [M + H]⁺ |
| 326 | 433.0385 | 434.0 | [M + H]⁺ |
| 327 | 433.1038 | 434.1 | [M + H]⁺ |
| 328 | 435.0586 | 505.93 | [M + H]⁺ |
| 329 | 435.0943 | 436.0 | [M + H]⁺ |
| 330 | 438.1492 | 439.5 | [M + H]⁺ |
| 331 | 448.1147 | 449.5 | [M + H]⁺ |
| 332 | 453.0993 | 454.1 | [M + H]⁺ |
| 333 | 457.0441 | 458.07 | [M + H]⁺ |
| 334 | 465.11 | 466.1 | [M + H]⁺ |
| 335 | 467.0915 | 468.0 | [M + H]⁺ |
| 336 | 468.0601 | 469.0 | [M + H]⁺ |
| 337 | 473.0987 | 473.88 | [M + H]⁺ |
| 338 | 487.1144 | 488.1 | [M + H]⁺ |
| 339 | 491.0893 | 492.06 | [M + H]⁺ |
| 340 | 491.0893 | 492.07 | [M + H]⁺ |
| 341 | 491.0893 | 492.03 | [M + H]⁺ |
| 342 | 499.1256 | 500.1 | [M + H]⁺ |
| 343 | 508.0429 | 508.74 | [M + H]⁺ |
| 344 | 518.0757 | 519.0 | [M + H]⁺ |
| 345 | 552.1021 | 553.14 | [M + H]⁺ |

### Synthesis of Compound 346 Via Stille Coupling to 2-Chloro-Oxazole Intermediate.

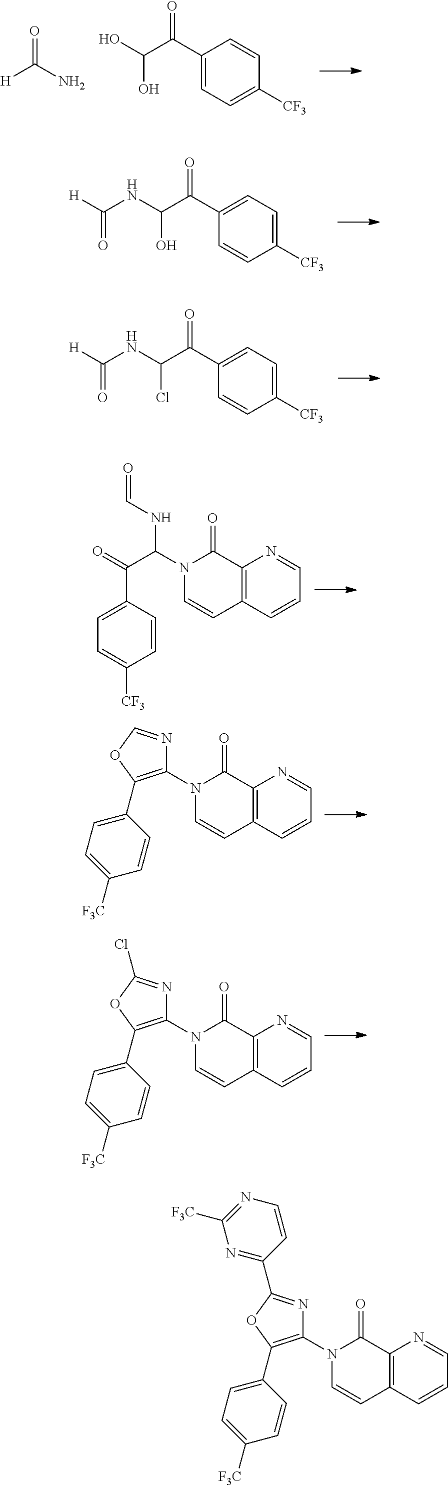

### N-{1-hydroxy-2-oxo-2-[4-(trifluoromethyl)phenyl]ethyl}formamide

Formamide (57.7 mL, 1.45 mol, 4.0 eq) and 2,2-dihydroxy-1-(4-(trifluoromethyl)phenyl)ethan-1-one (84.2 g, 363 mmol, 1.0 eq) were dissolved in dioxane (976 mL). The resulting mixture was heated up to 100° C. degrees and stirred for 2 hours at that temperature. Dioxane was then removed under reduced pressure. The crude solid was dissolved in EtOAc and the organic phase was washed with sat. aq. NaHCO₃ (3 times). The solvent was evaporated and the solid was triturated with hexanes in EtOAc to give N-{1-hydroxy-2-oxo-2-[4-(trifluoromethyl)phenyl]ethyl}formamide (79.8 g, 323 mmol, 89%).

¹H NMR (300 MHz, DMSO-d6) δ 9.10-8.96 (m, 1H), 8.19-8.09 (m, 3H), 7.94-7.89 (m, 2H), 6.87 (d, J=7.3 Hz, 1H), 6.45-6.34 (m, 1H).

### N-(1-chloro-2-oxo-2-(4-(trifluoromethyl)phenyl)ethyl)formamide

N-{1-Hydroxy-2-oxo-2-[4-(trifluoromethyl)phenyl]ethyl}formamide (26.5 g, 107 mmol) was dissolved in dichloromethane (532 mL). Phosphorus pentachloride (24.6 g, 118 mmol, 1.1 eq) was added and the resulting mixture was stirred for 16 hours at ambient temperature. Solvent was removed under reduced pressure, and the crude solid was triturated with ethyl acetate to afford N-(1-chloro-2-oxo-2-(4-(trifluoromethyl)phenyl)ethyl)formamide (17.7 g, 66.6 mmol, 62%).

¹H NMR (300 MHz, DMSO-d6) δ 9.04 (d, J=8.5 Hz, 1H), 8.19-8.08 (m, 3H), 7.91 (s, 2H), 6.37 (d, J=8.6 Hz, 1H).

### N-(2-oxo-1-(8-oxo-1,7-naphthyridin-7(8H)-yl)-2-(4-(trifluoromethyl)phenyl)ethyl) formamide

7,8-Dihydro-1,7-naphthrydin-8-one (23.9 g, 163 mmol, 0.7 eq) was dissolved in dimethylformamide (1,241 mL) and triethylamine (163 mL, 1170 mmol, 5.0 eq) was added slowly. The resulting mixture was stirred at ambient temperature for 10 minutes. Next, N-(1-chloro-2-oxo-2-(4-(trifluoromethyl)phenyl)ethyl)formamide (62.1 g, 234 mmol, 1.0 eq) was added in portions and stirred at ambient temperature for 1.5 hours. The solvent was then removed under reduced pressure and the residue dried at 50° C. for 16 h. The resulting solid was suspended in water and then filtered and dried. The solid was then washed with a mixture of ether and ethyl acetate (8:2) and one more time with a small amount of MeOH to give N-[2-oxo-1-(8-oxo-7,8-dihydro-1,7-naphthyridin-7-yl)-2-[4-(trifluoromethyl)phenyl]ethyl]formamide (41.0 g, 109 mmol, 39%).

LCMS (ESI+): m/z 376.0 [M+H]⁺.

### 7-(5-(4-(trifluoromethyl)phenyl)oxazol-4-yl)-1,7-naphthyridin-8(7H)-one

N-[2-Oxo-1-(8-oxo-7,8-dihydro-1,7-naphthyridin-7-yl)-2-[4-(trifluoromethyl)phenyl]ethyl]formamide (41.0 g, 90.6 mmol, 1.0 eq) was dissolved in thionyl chloride (211 mL, 2.9 mol, 32.0 eq) and the resulting mixture was stirred at 60° C. for 2 hours. The reaction was quenched with methanol and the volatiles were removed under reduced pressure. The crude solid material was evaporated to dryness, triturated with methanol, and dried to afford 7-(5-(4-(trifluoromethyl)phenyl)oxazol-4-yl)-1,7-naphthyridin-8(7H)-one (14.8 g, 43%, UPLC purity: 88%). The filtrate from the methanol wash was concentrated and purified by silica gel column chromatography to afford an additional amount of 7-(5-(4-(trifluoromethyl)phenyl)oxazol-4-yl)-1,7-naphthyridin-8(7H)-one (5.0 g, 15%, UPLC purity: 99%).

¹H NMR (300 MHz, DMSO-d6) δ 8.87 (dd, J=4.4, 1.7 Hz, 1H), 8.79 (s, 1H), 8.26 (dd, J=8.1, 1.7 Hz, 1H), 7.82 (td, J=6.5, 3.3 Hz, 3H), 7.65 (dd, J=9.3, 7.7 Hz, 3H), 6.86 (d, J=7.4 Hz, 1H).

LCMS (ESI+): m/z 358.0 [M+H]⁺.

### 7-(2-chloro-5-(4-(trifluoromethyl)phenyl)oxazol-4-yl)-1,7-naphthyridin-8(7H)-one

7-(5-(4-(Trifluoromethyl)phenyl)oxazol-4-yl)-1,7-naphthyridin-8(7H)-one (5.4 g, 14 mmol, 1.0 eq) was dissolved in anhydrous dimethylformamide (150 mL) and anhydrous tetrahydrofuran (350 mL). The resulting solution was cooled down to −78° C. and lithium bis(trimethylsilyl)amide (1M THF, 28 mL, 28 mmol, 2.0 eq) was added dropwise. The mixture was stirred for 30 minutes, and hexachloroethane (6.49 g, 27.4 mmol, 1.96 eq) was then added in one portion. The reaction mixture was allowed to warm up to ambient temperature and stirred for 16 hours after which the reaction was quenched with MeOH and the solvent was evaporated. The crude solid was then purified by silica gel column chromatography (CH₂Cl₂ 100%) to give 7-(2-chloro-5-(4-(trifluoromethyl)phenyl)oxazol-4-yl)-1,7-naphthyridin-8(7H)-one (2.5 g, 6.38 mmol, 42%).

LCMS (ESI+): m/z 392.0 [M+H]⁺.

¹H NMR (300 MHz, DMSO-d₆) δ 8.87 (dd, J=4.4, 1.7 Hz, 1H), 8.25 (dd, J=8.1, 1.7 Hz, 1H), 7.87-7.80 (m, 3H), 7.67-7.59 (m, 3H), 6.87 (d, J=7.4 Hz, 1H).

### 7-(5-(4-(trifluoromethyl)phenyl)-2-(2-(trifluoromethyl)pyrimidin-4-yl)oxazol-4-yl)-1,7-naphthyridin-8(7H)-one. Compound 346

4-Chloro-2-(trifluoromethyl)pyrimidine (1.0 g, 5.48 mmol, 1.0 eq), tetrakis(triphenylphosphine)palladium (0.317 g, 0.274 mmol, 0.05 eq), and hexabutylditin (3.81 g, 6.57 mmol, 1.2 eq) were dissolved in dioxane (20.0 mL). The reaction mixture was heated to 85° C. overnight. After cooling to ambient temperature, the reaction was filtered through celite. The celite pad was washed with EtOAc and the collected filtrate evaporated to dryness to afford 4-(tributylstannyl)-2-(trifluoromethyl)pyrimidine (2.40 g, 5.48 mmol, 100%) as an oil which was used immediately in the next step. 7-(2-Chloro-5-(4-(trifluoromethyl)phenyl)oxazol-4-yl)-1,7-naphthyridin-8(7H)-one (1.0 g, 2.45 mmol, 1.0 eq) and 4-(tributylstannyl)-2-(trifluoromethyl)pyrimidine (2.14 g, 4.89 mmol, 2.0 eq) were dissolved in dimethylformamide (20.0 mL). The mixture was purged with argon for 20 minutes and tetrakis(triphenylphosphine)palladium (0.283 g, 0.245 mmol, 0.1 eq) and copper(I) iodide (0.047 g, 0.245 mmol, 0.1 eq) were added. The reaction was then stirred for 16 hours at 90° C. in a sealed tube. The reaction was filtered through a pad of celite. The celite was then washed with CH₂Cl₂ and then a mixture of CH₂Cl₂/MeOH. The collected filtrate was then evaporated to dryness and the crude solid was purified by preparative-HPLC to afford 7-(5-(4-(trifluoromethyl)phenyl)-2-(2-(trifluoromethyl)pyrimidin-4-yl)oxazol-4-yl)-1,7-naphthyridin-8(7H)-one (0.12 g, 10% yield).

LCMS (ESI+): m/z 504.1 [M+H]⁺.

¹H NMR (300 MHz, DMSO-d6): d 9.36 (d, J=5.3 Hz, 1H), 8.90 (dd, J=4.4, 1.7 Hz, 1H), 8.60 (d, J=5.2 Hz, 1H), 8.30 (dd, J=8.2, 1.7 Hz, 1H), 7.98-7.81 (m, 5H), 7.72 (d, J=7.4 Hz, 1H), 6.93 (d, J=7.4 Hz, 1H).

Additional Compounds were Prepared in a Similar Manner to Compound 346:

| | Exact mass | | LCMS |
|---|---|---|---|
| Compound | (g/mol) | LCMS | Assignment |
| 347 | 398.1179 | 399.03 | [M + H]⁺ |
| 348 | 398.1179 | 399.06 | [M + H]⁺ |
| 349 | 400.0727 | 401.0 | [M + H]⁺ |
| 350 | 400.0727 | 400.9 | [M + H]⁺ |
| 351 | 401.068 | 402.0 | [M + H]⁺ |
| 352 | 408.1023 | 408.97 | [M + H]⁺ |
| 353 | 414.0884 | 415.01 | [M + H]⁺ |
| 354 | 414.1128 | 415.03 | [M + H]⁺ |
| 355 | 418.0633 | 418.97 | [M + H]⁺ |
| 356 | 423.1132 | 424.07 | [M + H]⁺ |
| 357 | 434.0337 | 436.96 | [M + H]⁺ |
| 358 | 434.0991 | 435.09 | [M + H]⁺ |
| 359 | 434.0991 | 435.07 | [M + H]⁺ |
| 360 | 435.0943 | 436.02 | [M + H]⁺ |
| 361 | 435.0943 | 436.01 | [M + H]⁺ |
| 362 | 440.0743 | 441.08 | [M + H]⁺ |
| 363 | 441.0696 | 441.97 | [M + H]⁺ |
| 364 | 458.0991 | 458.79 | [M + H]⁺ |
| 365 | 458.0991 | 458.92 | [M + H]⁺ |
| 366 | 459.0943 | 460.2 | [M + H]⁺ |
| 367 | 468.0601 | 469.06 | [M + H]⁺ |
| 368 | 468.0601 | 469.08 | [M + H]⁺ |
| 369 | 490.0711 | 491.05 | [M + H]⁺ |
| 370 | 498.1304 | 499.17 | [M + H]⁺ |
| 371 | 502.0864 | 503.1 | [M + H]⁺ |
| 372 | 502.1365 | 502.86 | [M + H]⁺ |

### Synthesis of Compounds 124 and 373 Via Direct C—H Arylation at Oxazole C2 Using Aryl and Heteroaryl Halides.

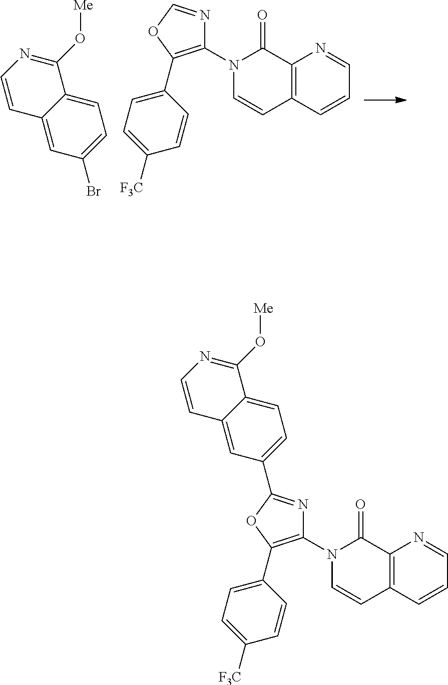

### 7-(2-(1-methoxyisoquinolin-6-yl)-5-(4-(trifluoromethyl)phenyl)oxazol-4-yl)-1,7-naphthyridin-8(7H)-one. Compound 124

7-(5-(4-(Trifluoromethyl)phenyl)oxazol-4-yl)-1,7-naphthyridin-8(7H)-one (8.70 g, 22.4 mmol, 1.0 eq), 6-bromo-1-methoxyisoquinoline (10.9 g, 44.8 mmol, 2.0 eq) and cesium carbonate (21.9 g, 67.2 mmol, 3.0 eq) were transferred to a reaction tube and anhydrous dimethylformamide (160 mL) was added. Next, the reaction mixture was degassed with argon for 10 min and CuI (0.426 g, 2.24 mmol, 0.1 eq) and 1,1′-Bis(diphenylphosphino)ferrocene palladium (II) dichloride (1.64 g, 2.24 mmol, 0.1 eq) were added. The reaction was stirred at 100° C. for 48 hours. The reaction mixture was then cooled too ambient temperature, filtered through a pad of celite and washed with CH₂Cl₂ and a mixture of CH₂Cl₂/MeOH. The filtrate was then evaporated to dryness and the crude solid purified by silica gel column chromatography (0-10% MeOH in DCM) to afford 7-[2-(1-methoxyisoquinolin-6-yl)-5-[4-(trifluoromethyl)phenyl]-1,3-oxazol-4-yl]-7,8-dihydro-1,7-naphthyridin-8-one (7.5 g, 14.6 mmol, 65%).

LCMS (ESI+): m/z 515.1, [M+H]⁺.

¹H NMR (300 MHz, DMSO-d6) δ 8.90 (dd, J=4.4, 1.7 Hz, 1H), 8.81 (d, J=1.5 Hz, 1H), 8.41-8.31 (m, 2H), 8.29 (dd, J=8.2, 1.7 Hz, 1H), 8.15 (d, J=5.9 Hz, 1H), 7.93-7.81 (m, 5H), 7.76 (d, J=7.4 Hz, 1H), 7.63 (d, J=5.9 Hz, 1H), 6.91 (d, J=7.4 Hz, 1H), 4.11 (s, 3H).

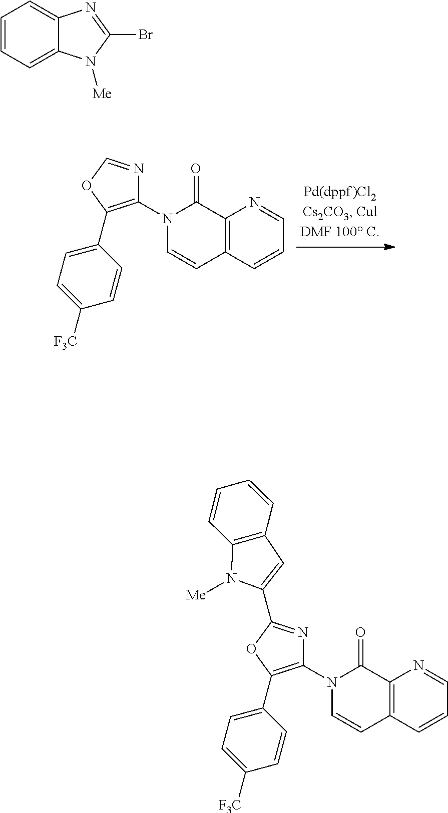 [SMILES: Cn1c(Br)nc2ccccc21 ; O=c1c2ncccc2ccn1-c1ncoc1-c1ccc(C(F)(F)F)cc1 ; Cn1c(-c2nc(-n3ccc4cccnc4c3=O)c(-c3ccc(C(F)(F)F)cc3)o2)cc2ccccc21]

### 7-[2-(1-methyl-1H-1,3-benzodiazol-2-yl)-5-[4-(trifluoromethyl)phenyl]-1,3-oxazol-4-yl]-7,8-dihydro-1,7-naphthyridin-8-one. Compound 373

7-{5-[4-(Trifluoromethyl)phenyl]-1,3-oxazol-4-yl}-7,8-dihydro-1,7-naphthyridin-8-one (0.051 g, 0.173 mmol, 1.0 eq.), 2-bromo-1-methyl-1H-1,3-benzodiazole (0.075 g, 0.345 mmol, 1.99 eq.) and Cesium carbonate (0.11 g, 0.518 mmol, 3.00 eq.) were added to a reaction tube and anhydrous dimethylformamide (2.0 mL) was added. Next, the reaction mixture was bubbled with argon for 10 minutes and copper (I) iodide (0.003 g, 0.016 mmol, 0.091 eq.) and 1,1′-bis(diphenylphosphino)ferrocene palladium (II) dichloride (0.013 g, 0.018 mmol, 0.103 eq.) were added. The reaction was stirred for 2 days at 100° C. The reaction mixture was filtered through celite and washed with dichloromethane and a mixture of dichloromethane/methanol (9/1 v/v). The resulting organic filtrate was concentrated under reduced pressure. This material was purified via preparative thin layer chromatography (4 elutions of DCM/MeOH: 100/0; 98/2 x2; 97/3) to give 7-[2-(1-methyl-1H-1,3-benzodiazol-2-yl)-5-[4-(trifluoromethyl)phenyl]-1,3-oxazol-4-yl]-7,8-dihydro-1,7-naphthyridin-8-one (0.012 g, 0.025 mmol, 14%).

LCMS (ESI+): m/z 488.1, [M+H]⁺.

¹H NMR (300 MHz, DMSO-d6) δ 8.90 (dd, J=4.4, 1.6 Hz, 1H), 8.29 (dd, J=8.1, 1.7 Hz, 1H), 7.98-7.69 (m, 8H), 7.54-7.32 (m, 2H), 6.93 (d, J=7.4 Hz, 1H), 4.26 (s, 3H).

Additional Compounds were Prepared in a Similar Manner to Compounds 124 & 373:

| | Exact mass | | LCMS |
|---|---|---|---|
| Compound | (g/mol) | LCMS | Assignment |
| 374 | 380.1273 | 381.1 | [M + H]+ |
| 375 | 404.0743 | 405.0 | [M + H]+ |
| 376 | 404.0743 | 405.0 | [M + H]+ |
| 377 | 412.1336 | 413.2 | [M + H]+ |
| 378 | 418.09 | 419.0 | [M + H]+ |
| 379 | 427.1445 | 428.1 | [M + H]+ |
| 380 | 434.1179 | 435.0 | [M + H]+ |
| 381 | 434.1179 | 435.0 | [M + H]+ |
| 382 | 437.1288 | 438.1 | [M + H]+ |
| 383 | 442.0877 | 443.0 | [M + H]+ |
| 384 | 443.1194 | 444.2 | [M + H]+ |
| 385 | 444.1056 | 445.2 | [M + H]+ |
| 386 | 446.1379 | 447.1 | [M + H]+ |
| 387 | 460.1535 | 461.1 | [M + H]+ |
| 388 | 462.1304 | 463.2 | [M + H]+ |
| 389 | 463.1445 | 464.4 | [M + H]+ |
| 390 | 464.104 | 465.0 | [M + H]+ |
| 391 | 465.1237 | 466.3 | [M + H]+ |
| 392 | 470.0849 | 471.0 | [M + H]+ |
| 393 | 470.0849 | 471.0 | [M + H]+ |
| 394 | 473.11 | 474.0 | [M + H]+ |
| 395 | 473.11 | 474.3 | [M + H]+ |
| 396 | 473.1852 | 474.3 | [M + H]+ |
| 397 | 474.1052 | 475.0 | [M + H]+ |
| 398 | 474.1052 | 474.8 | [M + H]+ |
| 399 | 475.1144 | 476.1 | [M + H]+ |
| 400 | 476.1485 | 477.1 | [M + H]+ |
| 401 | 479.1205 | 480.4 | [M + H]+ |
| 402 | 479.1569 | 480.1 | [M + H]+ |
| 403 | 482.119 | 483.0 | [M + H]+ |
| 404 | 485.11 | 485.9 | [M + H]+ |
| 405 | 485.11 | 486.1 | [M + H]+ |
| 406 | 485.11 | 486.1 | [M + H]+ |
| 407 | 485.11 | 486.2 | [M + H]+ |
| 408 | 485.11 | 486.0 | [M + H]+ |
| 409 | 487.1256 | 488.1 | [M + H]+ |
| 410 | 487.1256 | 488.2 | [M + H]+ |
| 411 | 487.1256 | 488.2 | [M + H]+ |
| 412 | 487.1256 | 488.1 | [M + H]+ |
| 413 | 487.1256 | 488.0 | [M + H]+ |
| 414 | 487.1256 | 488.1 | [M + H]+ |
| 415 | 487.1256 | 488.0 | [M + H]+ |
| 416 | 488.1209 | 489.2 | [M + H]+ |
| 417 | 488.1209 | 489.2 | [M + H]+ |
| 418 | 488.1209 | 489.2 | [M + H]+ |
| 419 | 488.1209 | 489.2 | [M + H]+ |
| 420 | 490.0711 | 491.0 | [M + H]+ |
| 421 | 490.0711 | 491.0 | [M + H]+ |
| 422 | 494.1024 | 495.2 | [M + H]+ |
| 423 | 495.1507 | 496.1 | [M + H]+ |
| 424 | 497.1111 | 498.1 | [M + H]+ |
| 425 | 498.0895 | 499.0 | [M + H]+ |
| 426 | 498.0895 | 499.0 | [M + H]+ |
| 427 | 498.1304 | 499.1 | [M + H]+ |
| 428 | 498.1304 | 499.1 | [M + H]+ |
| 429 | 501.1224 | 502.1 | [M + H]+ |
| 430 | 502.1053 | 503.2 | [M + H]+ |
| 431 | 502.1365 | 503.2 | [M + H]+ |
| 432 | 502.1365 | 503.2 | [M + H]+ |
| 433 | 502.1365 | 503.2 | [M + H]+ |
| 434 | 502.1617 | 503.2 | [M + H]+ |
| 435 | 503.1205 | 504.1 | [M + H]+ |
| 436 | 503.1205 | 504.1 | [M + H]+ |
| 437 | 503.1205 | 504.2 | [M + H]+ |
| 438 | 503.1205 | 504.0 | [M + H]+ |
| 439 | 504.1409 | 505.2 | [M + H]+ |
| 440 | 507.071 | 508.3 | [M + H]+ |
| 441 | 507.071 | 508.0 | [M + H]+ |
| 442 | 511.1256 | 512.2 | [M + H]+ |
| 443 | 511.1256 | 512.1 | [M + H]+ |
| 444 | 512.146 | 554.3 | [M + H + MeCN]+ |
| 445 | 513.13 | 514.1 | [M + H]+ |
| 446 | 513.1413 | 514.1 | [M + H]+ |
| 447 | 514.1253 | 515.1 | [M + H]+ |
| 448 | 514.1253 | 515.1 | [M + H]+ |
| 449 | 514.1253 | 515.4 | [M + H]+ |
| 450 | 514.1253 | 515.1 | [M + H]+ |
| 451 | 514.1365 | 515.1 | [M + H]+ |
| 452 | 515.1205 | 516.1 | [M + H]+ |
| 453 | 516.1522 | 517.1 | [M + H]+ |
| 454 | 516.1773 | 517.2 | [M + H]+ |
| 455 | 517.1362 | 518.2 | [M + H]+ |
| 456 | 526.1617 | 527.2 | [M + H]+ |
| 457 | 528.1409 | 529.9 | [M + H]+ |
| 458 | 528.1409 | 529.1 | [M + H]+ |
| 459 | 530.1202 | 531.5 | [M + H]+ |
| 460 | 537.1224 | 538.1 | [M + H]+ |
| 461 | 541.0973 | 542.2 | [M + H]+ |
| 462 | 544.1522 | 545.0 | [M + H]+ |
| 463 | 545.1311 | 546.0 | [M + H]+ |
| 464 | 548.1272 | 549.2 | [M + H]+ |
| 465 | 623.178 | 624.2 | [M + H]+ |

### Synthesis of Aryl Bromides for Direct Arylation

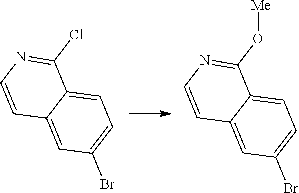 [SMILES: Clc1nccc2cc(Br)ccc12 ; COc1nccc2cc(Br)ccc12]

### 6-bromo-1-methoxyisoquinoline

6-Bromo-1-chloroisoquinoline (20.0 g, 82.5 mmol, 1.0 eq) and sodium methoxide (13.4 g, 247 mmol, 3.0 eq) were dissolved in a mixture of anhydrous methanol (33.4 mL, 825 mmol, 10.0 eq) and dioxane (220 mL). The mixture was stirred for 16 hours at 100° C. The reaction was cooled and another portion of sodium methoxide (8.02 g, 148 mmol, 3.0 eq) and methanol (0.194 mL, 4.80 mmol, 5.00 eq) in dioxane (1.38 mL) were added, and the reaction mixture was heated for 16 hours at 100° C. The reaction was then allowed to cool and diluted with a mixture of ethyl acetate and water. The product was extracted 3 times with ethyl acetate and the combined organic fraction dried over sodium sulfate. The sodium sulfate was filtered off and the filtrate evaporated to dryness to afford 6-bromo-1-methoxyisoquinoline (19.7 g, 82.7 mmol, 98%).

LCMS (ESI+): m/z 239.9, [M+H]⁺.

¹H NMR (300 MHz, DMSO-d6): d 8.21 (d, J=2.0 Hz, 1H), 8.14-8.01 (m, 2H), 7.75 (dd, J=8.8, 2.0 Hz, 1H), 7.38 (dd, J=5.9, 0.8 Hz, 1H), 4.06 (s, 3H).

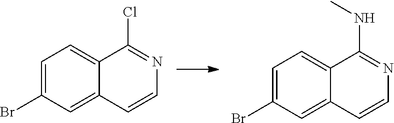

### 6-bromo-N-methylisoquinolin-1-amine

To a slurry mixture of 6-bromo-1-chloro-isoquinoline, (2.00 g, 16.5 mL, 1 Eq, 8.25 mmol) and methylamine (19.4 g, 50 mL, 33% Wt. in EtOH, 25 Eq, 206 mmol) was heated at 80° C. for 12 hours in a high-pressure flask. After 12 hours, the reaction was cooled to room temperature and concentrated. The resulting residue was loaded onto silica and purified by normal phase column chromatography (0 to 50% MeOH with 0.1% NH₃ in DCM) to yield 6-bromo-N-methylisoquinolin-1-amine (1.5 g, 6.3 mmol, 77%) as a pink solid.

LCMS (ESI+): 237.1 [M+H]⁺.

### Synthesis of 6-bromo-1-(1,1-difluoroethyl)isoquinoline

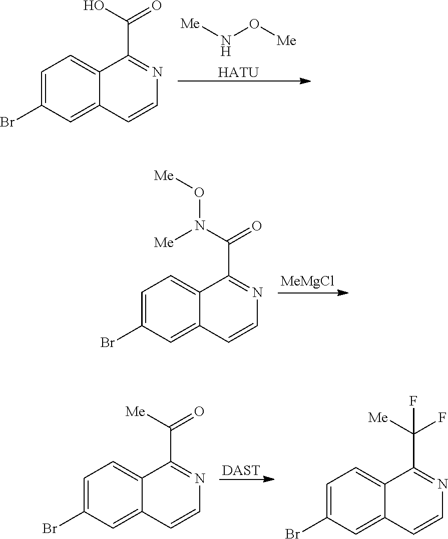

### 6-bromo-N-methoxy-N-methylisoquinoline-1-carboxamide

To a solution of 6-bromoisoquinoline-1-carboxylic acid (1.05 g, 3.97 mmol, 1.0 eq), N-[(dimethylamino)-1H-1,2,3-triazolo-[4,5-b]pyridin-1-ylmethylene]-N-methylmethanaminium hexafluorophosphate N-oxide (HATU) (1.86 g, 4.76 mmol, 1.2 eq), and methoxy(methyl)amine hydrochloride (0.774 g, 7.94 mmol, 2.0 eq) in anhydrous dimethylformamide (30.0 mL) was added N,N-diisopropylethylamine (3.46 mL, 19.8 mmol, 5.0 eq). The reaction mixture was stirred at ambient temperature overnight. The reaction was quenched by addition of water. The product was extracted with ethyl acetate (3 times). The combined organic layers were washed with brine, dried over sodium sulfate, and concentrated. The crude material was purified via normal phase column chromatography (SiO₂, 50% EtOAc in hexanes) to afford 6-bromo-N-methoxy-N-methylisoquinoline-1-carboxamide (1.04 g, 3.51 mmol, 86%) as a white solid.

¹H NMR (300 MHz, DMSO-d6): d 8.56 (d, J=5.6 Hz, 1H), 8.39 (s, 1H), 7.91 (d, J=5.8 Hz, 1H), 7.85 (s, 2H), 3.47 (s, 3H), 3.41 (s, 3H).

### 1-(6-bromoisoquinolin-1-yl)ethan-1-one

6-Bromo-N-methoxy-N-methylisoquinoline-1-carboxamide (1.03 g, 3.39 mmol, 1.0 eq) was dissolved in anhydrous tetrahydrofuran (20.0 mL), and the mixture was cooled to −78° C. Methylmagnesium chloride (3 M in THF, 1.69 mL, 5.08 mmol, 1.5 eq) was added dropwise and the reaction was allowed to warm to ambient temperature. The reaction was stirred for 1 hour. The reaction was quenched by the addition of water. The mixture was diluted with ethyl acetate and the layers were separated. The aqueous layer was extracted with ethyl acetate (2 times) and the combined organic layers were washed with brine once, dried over sodium sulfate, and concentrated. The crude material was purified by normal phase column chromatography (SiO₂, 20% EtOAc in hexanes) to afford 1-(6-bromoisoquinolin-1-yl)ethan-1-one (0.702 g, 2.81 mmol, 83%) as a white solid.

¹H NMR (300 MHz, DMSO-d6): d 8.74 (d, J=9.2 Hz, 1H), 8.68 (d, J=5.5 Hz, 1H), 8.41 (d, J=2.1 Hz, 1H), 8.08 (d, J=5.6 Hz, 1H), 7.91 (dd, J=9.2, 2.1 Hz, 1H), 2.77 (s, 3H).

### 6-bromo-1-(1,1-difluoroethyl)isoquinoline

To a solution of 1-(6-bromoisoquinolin-1-yl)ethan-1-one (0.35 g, 1.40 mmol, 1.0 eq) in dichloroethane (1.05 mL) was added diethylaminosulfur trifluoride (DAST) (1.48 mL, 11.2 mmol, 8.00 eq) and the reaction was heated to 75° C. for 16 hours. The reaction was cooled to ambient temperature and poured onto ice. The resulting mixture was extracted with chloroform (3 times). The combined organic layers were washed with brine, dried over sodium sulfate, and concentrated. The crude material was purified by normal phase column chromatography (silica gel, 0-10% EtOAc in hexanes) to afford 6-bromo-1-(1,1-difluoroethyl)isoquinoline (0.272 g, 1.0 mmol, 71%) as a yellow oil.

¹H NMR (300 MHz, DMSO-d6): d 8.59 (d, J=5.6 Hz, 1H), 8.45 (d, J=2.1 Hz, 1H), 8.36 (dt, J=9.2, 2.4 Hz, 1H), 8.03 (d, J=5.7 Hz, 1H), 7.93 (dd, J=9.2, 2.1 Hz, 1H), 2.21 (t, J=19.8 Hz, 3H).

### 6-bromo-1-(2-fluoropropan-2-yl)isoquinoline

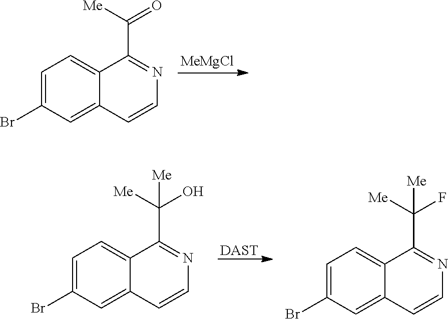

### 2-(6-bromoisoquinolin-1-yl)propan-2-ol

1-(6-Bromoisoquinolin-1-yl)ethan-1-one (0.419 g, 1.34 mmol, 1.00 eq) was dissolved in anhydrous tetrahydrofuran (6.7 mL) and cooled to −78° C. Methylmagnesium chloride (3 M in THF, 0.67 mL, 2.01 mmol, 1.50 eq) was added dropwise, and the reaction was allowed to warm to ambient temperature and stirred for 1 hour. The reaction was quenched by the addition of 5 mL of water. The resulting mixture was diluted with ethyl acetate and the layers were separated. The aqueous layer was washed with ethyl acetate (2 times) and the combined organic layer were washed with brine, dried over sodium sulfate, and concentrated. The crude material was purified by silica gel column chromatography (10-35% EtOAc in hexanes) to afford 2-(6-bromoisoquinolin-1-yl)propan-2-ol (0.313 g, 1.18 mmol, 75%) as a dark yellow oil.

¹H NMR (300 MHz, DMSO-d6) d 9.06 (d, J=9.3 Hz, 1H), 8.42 (d, J=5.6 Hz, 1H), 8.26 (d, J=2.2 Hz, 1H), 7.75 (dd, J=9.3, 2.2 Hz, 1H), 7.70 (d, J=5.5 Hz, 1H), 5.73 (s, 1H), 1.66 (s, 6H).

### 6-bromo-1-(2-fluoropropan-2-yl)isoquinoline

To a solution of 2-(6-bromoisoquinolin-1-yl)propan-2-ol (0.306 g, 0.977 mmol, 1.0 eq) in dichloromethane (2.08 mL) was added at 0° C. diethylaminosulfur trifluoride (DAST) (0.194 mL, 1.47 mmol, 1.50 eq) and the reaction was stirred for 2 hours. The reaction was quenched by the addition of saturated sodium bicarbonate solution and was extracted with ethyl acetate (3 times). The combined organic phase layer was washed with brine, dried over sodium sulfate, and concentrated. The crude material was purified by silica gel column chromatography (0-10% EtOAc in hexanes) to afford 6-bromo-1-(2-fluoropropan-2-yl)isoquinoline (0.144 g, 0.537 mmol, 47%) as a dark orange oil.

¹H NMR (300 MHz, DMSO-d6) d: 8.53-8.49 (m, 2H), 8.35 (d, J=2.1 Hz, 1H), 7.85-7.81 (m, 2H), 1.88 (d, J=22.0 Hz, 6H).

### 6-bromo-1-ethylisoquinoline

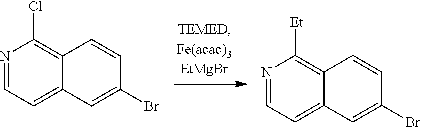 [SMILES: Clc1nccc2cc(Br)ccc12 ; CCc1nccc2cc(Br)ccc12]

6-Bromo-1-chloroisoquinoline (0.5 g, 2.06 mmol, 1.0 eq) was dissolved in anhydrous tetrahydrofuran (5.0 mL) and N,N,N′,N′-tetramethyl ethylenediamine (0.12 g, 1.03 mmol, 0.5 eq) was added followed by iron(III) acetylacetonate (0.007 g, 0.021 mmol, 0.01 eq). The resulting mixture was cooled to 0° C. and ethyl magnesium chloride (2 M in THF, 0.243 mL, 2.68 mmol, 1.3 eq) was added. The reaction mixture was stirred for 2 hours at 0° C. The reaction was quenched by the addition of a 5% aqueous solution of citric acid and the organic layer is separated. The organic layer was washed with brine, dried over sodium sulfate, filtered, and concentrated. The crude material was purified by silica gel column chromatography (100% DCM) to give 6-bromo-1-ethylisoquinoline (0.213 g, 0.902 mmol, 42%).

¹H NMR (300 MHz, DMSO-d₆) δ 8.44 (d, J=5.7 Hz, 1H), 8.27 (d, J=2.1 Hz, 1H), 8.25-8.20 (m, 1H), 7.79 (dd, J=9.0, 2.1 Hz, 1H), 7.66 (d, J=5.8 Hz, 1H), 3.28 (q, J=7.5 Hz, 2H), 1.33 (t, J=7.5 Hz, 3H).

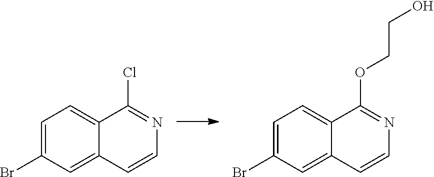 [SMILES: Clc1nccc2cc(Br)ccc12 ; OCCOc1nccc2cc(Br)ccc12]

### 2-((6-bromoisoquinolin-1-yl)oxy)ethan-1-ol

A mixture of 6-bromo-1-chloroisoquinoline (1.00 g, 8.25 mL, 1 Eq, 4.12 mmol), potassium 2-methylpropan-2-olate (463 mg, 4.12 mL, 1 molar in THF, 1 Eq, 4.12 mmol), and ethane-1,2-diol (256 g, 230 mL, 1000 Eq, 4.12 mol) in DMF (50 mL) was heated to 80° C. for 12 hours in a high-pressure flask. After 12 h, the reaction was let cool to room temperature and concentrated. The resulting residue was loaded onto silica and purified on silica gel column chromatography (0 to 50% MeOH with 0.1% ammonium hydroxide in DCM) to yield 2-((6-bromoisoquinolin-1-yl)oxy)ethan-1-ol (201 mg, 750 μmol, 18.2%) as a pink solid

LCMS (ESI+): m/z 268.0, [M+H]⁺

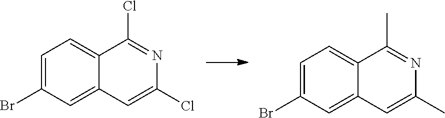

### 6-chloro-1,3-dimethylisoquinoline

1,3,6-Trichloroisoquinoline (2.00 g, 86.0 mL, 1 Eq, 8.60 mmol) was combined with THF and iron tri(4-methoxypent-3-en-2-one) (608 mg, 154 μL, 0.2 Eq, 1.72 mmol) was added. TMEDA (1.00 g, 1.29 mL, 1 Eq, 8.60 mmol) was then added. The resulting mixture was cooled to 0° C. and methylmagnesium chloride (3 M in THF, 2.57 g, 11.5 mL, 4.0 Eq, 34.4 mmol) was added at 0° C. After 1 hour at 0° C., additional methylmagnesium chloride (3 M in THF, 2.57 g, 11.5 mL, 4.0 Eq, 34.4 mmol) was added and the mixture was stirred for 30 minutes at the same temperature. At this time, the reaction was heated to 50° C. for 16 hours. Upon completion, ammonium chloride was added and the organic phase was separated. The organic phase was washed with brine (210 mL) and dried over sodium sulfate. The resulting solid was triturated with DCM to obtain 6-chloro-1,3-dimethylisoquinoline (600 mg, 3.13 mmol, 36.4%) as a white solid.

LCMS (ESI+): m/z 192.1, [M+H]⁺

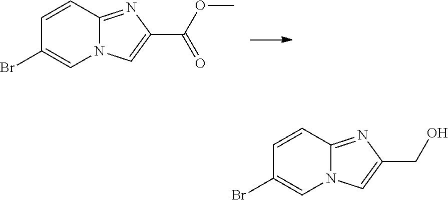

### (6-bromoimidazo[1,2-α]pyridin-2-yl)methanol

A mixture of methyl 6-bromoimidazo[1,2-α]pyridine-2-carboxylate (1.00 g, 1 Eq, 3.92 mmol), 1:2 THF:MeOH (24.5 mL), and calcium chloride (1.74 g, 4 Eq, 15.7 mmol) was cooled to 0° C. Sodium borohydride (742 mg, 5 Eq, 19.6 mmol) was added slowly and the reaction was warmed to room temperature and stirred for 2 hours. The reaction mixture was quenched by slow addition of water (500 mL). The aqueous layer was extracted with chloroform (3×150 mL). The combined organic layers were dried over anhydrous Na₂SO₄ and concentrated to obtain (6-bromoimidazo[1,2-α]pyridin-2-yl)methanol (300 mg, 1.32 mmol, 33.7%) as an off white solid

LCMS (ESI+): m/z 227.0, [M+H]⁺.

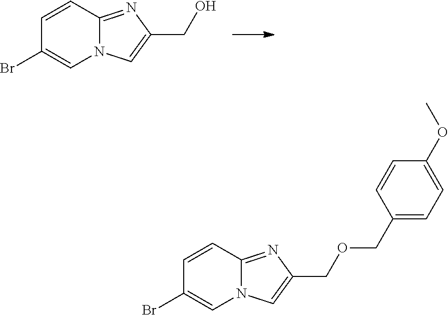 [SMILES: OCc1cn2cc(Br)ccc2n1 ; COc1ccc(COCc2cn3cc(Br)ccc3n2)cc1]

### 6-bromo-2-(((4-methoxybenzyl)oxy)methyl)imidazo[1,2-α]pyridine

To a solution of (6-bromoimidazo[1,2-α]pyridin-2-yl)methanol (150 mg, 1 Eq, 661 μmol) in DCM (2 mL) was added thionyl chloride (94.3 mg, 57.9 μL, 1.2 Eq, 793 μmol) at 5° C. The cold bath was removed, and the reaction was allowed to stir at room temperature for 30 minutes. The reaction was quenched with aqueous sodium bicarbonate. The organic layer was separated, washed with brine, dried, and concentrated to give the intermediate compound which was used directly. (4-Methoxyphenyl)methanol (137 mg, 121 μL, 1.5 Eq, 991 μmol) was dissolved in THF (1 mL) and the mixture was cooled to −78° C. NaH (39.6 mg, 60% Wt % in mineral oil, 1.5 Eq, 991 μmol) was suspended in THF (1 mL) and added to the mixture. The intermediate compound in 1 mL of THF was then added and the reaction was stirred at room temperature for 16 hours. The crude reaction was quenched with aqueous ammonium chloride and extracted with DCM. The organic extracts were dried over MgSO₄, filtered, and concentrated. The resulting material was purified on normal phase column chromatography (silica gel, 0-10% MeOH in DCM) to obtain 6-bromo-2-(((4-methoxybenzyl)oxy)methyl)imidazo[1,2-α]pyridine (160 mg, 461 μmol, 69.8%) as a brown solid

LCMS (ESI+): 347.0, [M+H]⁺.

### 7-(2-(2-(((4-methoxybenzyl)oxy)methyl)imidazo[1,2-α]pyridin-6-yl)-5-(4-(trifluoromethyl)phenyl)oxazol-4-yl)-1,7-naphthyridin-8(7H)-one

Synthesis of Compound 466.

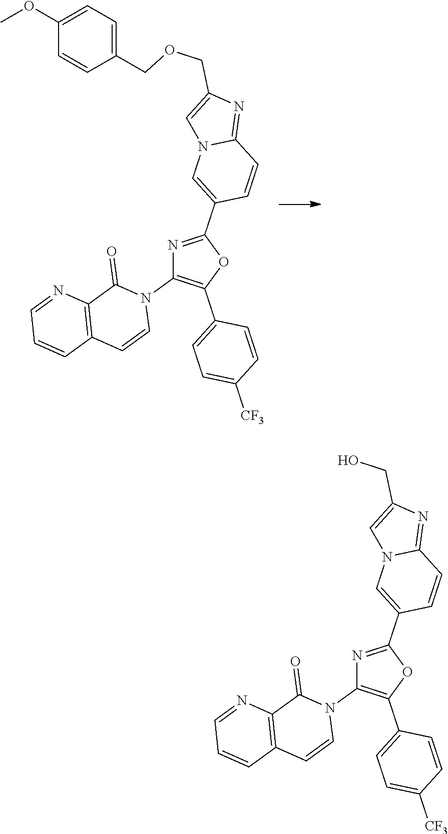 [SMILES: COc1ccc(COCc2cn3cc(-c4nc(-n5ccc6cccnc6c5=O)c(-c5ccc(C(F)(F)F)cc5)o4)ccc3n2)cc1 ; O=c1c2ncccc2ccn1-c1nc(-c2ccc3nc(CO)cn3c2)oc1-c1ccc(C(F)(F)F)cc1]

### 7-(2-(2-(hydroxymethyl)imidazo[1,2-α]pyridin-6-yl)-5-(4-(trifluoromethyl)phenyl)oxazol-4-yl)-1,7-naphthyridin-8(7H)-one. Compound 466

7-(2-(2-(((4-Methoxybenzyl)oxy)methyl)imidazo[1,2-α]pyridin-6-yl)-5-(4-(trifluoromethyl)phenyl)oxazol-4-yl)-1,7-naphthyridin-8(7H)-one (25 mg, 1 Eq, 40 μmol) was dissolved in TFA (740 mg, 500 μL, 160 Eq, 6.49 mmol), and the mixture was stirred at room temperature for 15 minutes. The reaction was then quenched with aqueous sodium bicarbonate and extracted with DCM. The organic extracts were concentrated onto silica gel and purified by silica gel column chromatography (0-10% MeOH in DCM with 0.1% ammonium hydroxide) to obtain 7-(2-(2-(hydroxymethyl)imidazo[1,2-α]pyridin-6-yl)-5-(4-(trifluoromethyl)phenyl) oxazol-4-yl)-1,7-naphthyridin-8(7H)-one (5.0 mg, 0.01 mmol, 20%) as a white solid.

LCMS (ESI+): 504, [M+H]⁺.

1H NMR: (400 MHz, DMSO-d6) δ=9.60 (s, 1H), 8.90-8.89 (d, J=4.0 Hz, 1H), 8.30-8.28 (d, J=8.0 Hz, 1H), 8.00 (s, 1H), 7.89-7.79 (m, 6H), 7.74-7.72 (d, J=7.2 Hz, 1H), 7.70-7.67 (d, J=9.2 Hz, 1H), 6.91-6.89 (d, J=7.2, 1H), 5.35-5.33 (t, J=5.6 Hz, 1H), 4.66-4.64 (d, J=5.6 Hz, 2H).

Synthesis of Compound 467 Via Suzuki Coupling of Pinacol Boronate Ester to 2-halo-oxazole Mediated by XPhos-Palladium Precatalyst.

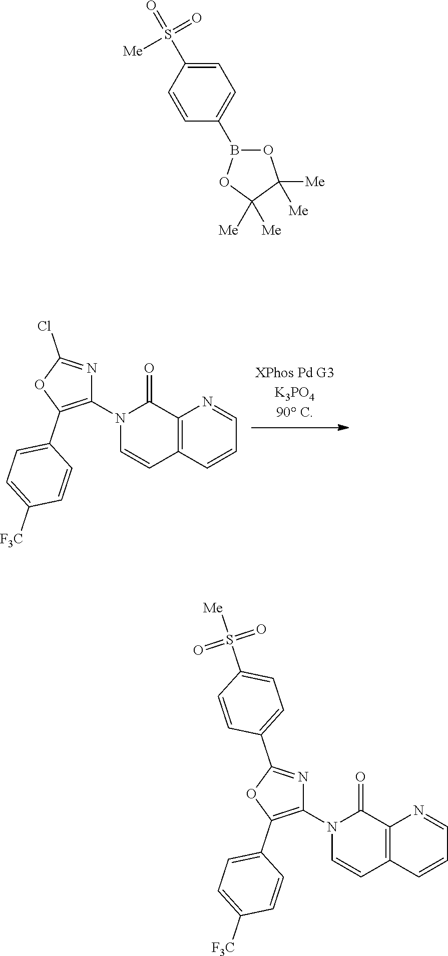 [SMILES: CC1(C)OB(c2ccc(S(C)(=O)=O)cc2)OC1(C)C ; O=c1c2ncccc2ccn1-c1nc(Cl)oc1-c1ccc(C(F)(F)F)cc1 ; CS(=O)(=O)c1ccc(-c2nc(-n3ccc4cccnc4c3=O)c(-c3ccc(C(F)(F)F)cc3)o2)cc1]

### 7-[2-(4-methanesulfonylphenyl)-5-[4-(trifluoromethyl)phenyl]-1,3-oxazol-4-yl]-7,8-dihydro-1,7-naphthyridin-8-one. Compound 467

7-{2-Chloro-5-[4-(trifluoromethyl)phenyl]-1,3-oxazol-4-yl}-7,8-dihydro-1,7-naphthyridin-8-one (0.075 g, 0.183 mmol, 1.0 eq), 4-methylsulfonylphenylboronic acid, pinacol ester (0.104 g, 0.367 mmol, 2.0 eq) and potassium phosphate tribasic (0.078 g, 0.367 mmol, 2.0 eq) were dissolved in a mixture of tetrahydrofuran (1.97 mL) and water (0.51 mL). The mixture was purged with argon for 10 minutes and XPhos Pd G3 (0.008 g, 0.009 mmol, 0.05 eq) was added. The reaction tube was sealed, and the mixture was stirred for 16 hours at 90° C. The reaction mixture was cooled to room temperature and brine (30 ml) and dichloromethane (30 ml) were added. The layers were separated, and the aqueous layer was extracted twice with dichloromethane (25 ml). The combined organic layers were dried over sodium sulfate, filtered, and concentrated to give crude material. The product was triturated with ethyl acetate. The resulting precipitate was collected by vacuum filteration and washed with ethyl acetate to give 7-[2-(4-methanesulfonylphenyl)-5-[4-(trifluoromethyl)phenyl]-1,3-oxazol-4-yl]-7,8-dihydro-1,7-naphthyridin-8-one (0.065 g, 0.18 mmol, 69%).

LCMS (ESI+): m/z=612.09, [M+H]⁺.

¹H NMR (300 MHz, DMSO-d₆) δ 8.90 (dd, J=4.4, 1.5 Hz, 1H), 8.45 (d, J=8.4 Hz, 2H), 8.29 (dd, J=8.1, 1.5 Hz, 1H), 8.18 (d, J=8.5 Hz, 2H), 7.85 (dd, J=8.9, 4.9 Hz, 5H), 7.74 (d, J=7.4 Hz, 1H), 6.92 (d, J=7.5 Hz, 1H), 3.35 (s, 3H).

Alternate Procedure for Suzuki Coupling of Pinacol Boronate to 2-halo-oxazole with Pd(dppf)Cl₂

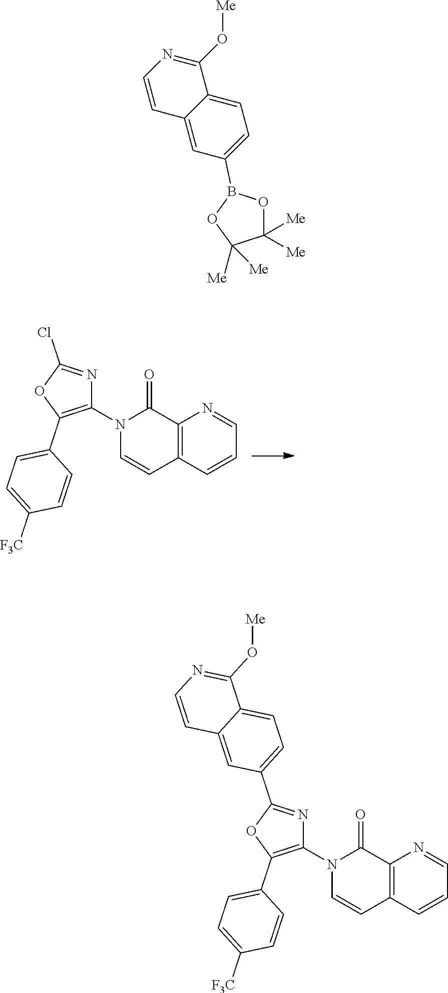 [SMILES: COc1nccc2cc(B3OC(C)(C)C(C)(C)O3)ccc12 ; O=c1c2ncccc2ccn1-c1nc(Cl)oc1-c1ccc(C(F)(F)F)cc1 ; COc1nccc2cc(-c3nc(-n4ccc5cccnc5c4=O)c(-c4ccc(C(F)(F)F)cc4)o3)ccc12]

### 7-(2-(1-methoxyisoquinolin-6-yl)-5-(4-(trifluoromethyl)phenyl)oxazol-4-yl)-1,7-naphthyridin-8(7H)-one. Compound 124

1-Methoxy-6-(4,4,5,5-tetramethyl-1,3,2-dioxaborolan-2-yl)isoquinoline (28 mg, 1.3 eq, 0.10 mmol) and Cs₂CO₃ (50 mg, 2 eq, 0.15 mmol) were dissolved in 1,4-dioxane (0.5 mL). The mixture was degassed with nitrogen for 15 minutes. Then, Pd(dppf)Cl₂·CH₂Cl₂ (9.4 mg, 0.15 Eq) and 7-(2-chloro-5-(4-(trifluoromethyl)phenyl)oxazol-4-yl)-1,7-naphthyridin-8(7H)-one (30 mg, 1 Eq, 77 μmol) were added to the reaction mixture and allowed to stir at 90° C. for 3 h. The crude mixture was then absorbed onto silica and the product purified by silica gel column chromatography (10% MeOH in DCM) to provide 7-(2-(1-methoxyisoquinolin-6-yl)-5-(4-(trifluoromethyl)phenyl)oxazol-4-yl)-1,7-naphthyridin-8(7H)-one (31 mg, 60 μmol, 79%).

LCMS (ESI+): m/z 515.1, [M+H]⁺.

¹H NMR (300 MHz, DMSO-d6) δ 8.90 (dd, J=4.4, 1.7 Hz, 1H), 8.81 (d, J=1.5 Hz, 1H), 8.41-8.31 (m, 2H), 8.29 (dd, J=8.2, 1.7 Hz, 1H), 8.15 (d, J=5.9 Hz, 1H), 7.93-7.81 (m, 5H), 7.76 (d, J=7.4 Hz, 1H), 7.63 (d, J=5.9 Hz, 1H), 6.91 (d, J=7.4 Hz, 1H), 4.11 (s, 3H).

### Pinacol Borane Intermediate Example Syntheses

### 7-fluoro-1-methoxy-6-(4,4,5,5-tetramethyl-1,3,2-dioxaborolan-2-yl)isoquinoline

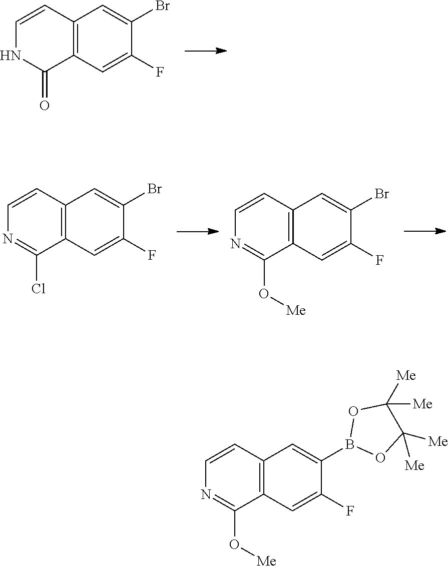 [SMILES: O=c1[nH]ccc2cc(Br)c(F)cc12 ; Fc1cc2c(Cl)nccc2cc1Br ; COc1nccc2cc(Br)c(F)cc12 ; COc1nccc2cc(B3OC(C)(C)C(C)(C)O3)c(F)cc12]

### 6-bromo-1-chloro-7-fluoroisoquinoline

6-Bromo-7-fluoro-2H-isoquinolin-1-one (2.0 g, 8.26 mmol, 1.0 eq) and diisopropylamine

(3.47 ml, 24.8 mmol, 3.0 eq) were dissolved in anhydrous toluene (26.0 mL) and phosphorus oxychloride (2.32 mL, 24.8 mmol, 3 eq) was added. The suspension was heated to 130° C. for 16 hours. The reaction mixture was allowed to cool to room temperature and then quenched with water. The mixture was extracted with EtOAc, and the combined organic extracts were dried over sodium sulphate, filtered, and evaporated. The crude material was purified by normal phase column chromatography (0-5% EtOAc in hexanes) to give 6-bromo-1-chloro-7-fluoroisoquinoline (1.83 g, 7.03 mmol, 84%).

LCMS (ESI+): m/z 261.9, [M+H]⁺ RT=4.02 min.

### 6-bromo-7-fluoro-1-methoxyisoquinoline

6-bromo-1-chloro-7-fluoroisoquinoline (1.83 g, 7.03 mmol, 1.0 eq), sodium methoxide (1.14 g, 21.1 mmol, 3.00 eq), and anhydrous methanol (2.84 mL, 70.2 mmol, 10.0 eq) in anhydrous dioxane (20.1 mL) were heated at reflux for 6 hours. At this time, an additional 1.5 equivalents of sodium methoxide and 5 equivalents of MeOH were added, and the reaction was stirred for 16 hours at reflux. The reaction was quenched with water and extracted 3 times with ethyl acetate. The combined organic layers were dried over sodium sulfate, filtered, and concentrated to give 6-bromo-7-fluoro-1-methoxyisoquinoline (1.69 g, 6.6 mmol, 66%) with 70% UPLC purity, that was used in the next step without further purification.

LCMS (ESI+): m/z 257.95, [M+H]⁺ RT=4.32 min, 70% purity.

### 7-fluoro-1-methoxy-6-(4,4,5,5-tetramethyl-1,3,2-dioxaborolan-2-yl)isoquinoline

Under inert atmosphere, 6-bromo-7-fluoro-1-methoxyisoquinoline (1.71 g, 4.69 mmol, 1.0 eq), bis(pinacolato)diboron (1.79 g, 7.03 mmol, 1.5 eq), and potassium acetate (0.246 g, 2.51 mmol, 2.00 eq) were suspended in anhydrous dioxane (12.0 mL). The mixture was purged with argon for 10 minutes and 1,1′-bis(diphenylphosphino)ferrocene palladium (II) dichloride (0.171 g, 0.234 mmol, 0.05 eq) was added and reaction tube was sealed. The reaction was then stirred at 90° C. for 16 hours. The reaction mixture was filtered through celite, and the celite pad was washed with EtOAc, DCM, and mixture of DCM/MeOH. The combined solvents were evaporated and the residue was purified by normal phase column chromatography (0-2% MeOH in DCM) to give 7-fluoro-1-methoxy-6-(4,4,5,5-tetramethyl-1,3,2-dioxaborolan-2-yl)isoquinoline (1.39 g, 4.59 mmol, 78%) with ˜80% purity according to NMR spectrum.

¹H NMR (300 MHz, DMSO-d6) d 8.30 (d, J=5.8 Hz, 1H), 8.02 (d, J=5.8 Hz, 1H), 7.74 (d, J=9.9 Hz, 1H), 7.54 (dd, J=6.0, 0.8 Hz, 1H), 4.06 (d, J=0.7 Hz, 4H), 1.35 (s, 12H).

### 2-ethyl-5-(4,4,5,5-tetramethyl-1,3,2-dioxaborolan-2-yl)-2H-indazole

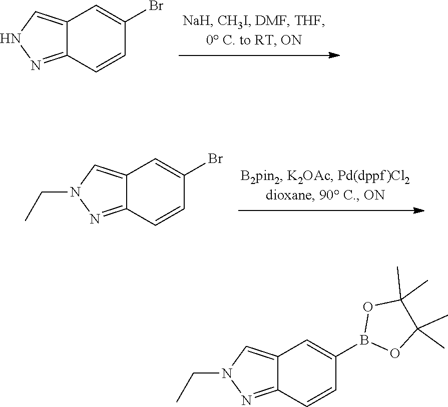 [SMILES: Brc1ccc2n[nH]cc2c1 ; CCn1cc2cc(Br)ccc2n1 ; CCn1cc2cc(B3OC(C)(C)C(C)(C)O3)ccc2n1]

### 5-bromo-2-ethyl-2H-indazole

5-Bromo-2H-indazole (1.0 g, 5.08 mmol, 1.0 eq) was added to anhydrous dimethylformamide (10.0 mL) and anhydrous tetrahydrofuran (10.0 mL). The reaction mixture was cooled to 0° C. and sodium hydride (0.304 g, 60 wt % in mineral oil, 7.60 mmol, 1.50 eq) was added. The reaction mixture was stirred for 15 minutes at 0° C. Next, iodoethane (0.816 mL, 10.2 mmol, 2.0 eq) was added slowly, and the mixture was stirred for 15 minutes at 0° C. After that time, the reaction mixture was allowed to warm to room temperature and stir for 16 hours. To the reaction mixture was added ethyl acetate, and the mixture was washed 3 times with brine. The organic layer was dried over sodium sulfate and evaporated. The crude material was purified by silica gel column chromatography (5-20% EtOAc in hexanes) to give 5-bromo-2-ethyl-2H-indazole (0.35 g, 1.56 mmol, 30%) and 5-bromo-1-ethyl-1H-indazole- (0.69 g, 3.07 mmol, 60%).

¹H NMR (400 MHz, DMSO-d6) d 8.06-7.97 (m, 2H), 7.69 (dt, J=8.9, 0.9 Hz, 1H), 7.49 (dd, J=8.9, 1.9 Hz, 1H), 4.44 (d, J=7.2 Hz, 2H), 1.38 (t, J=7.2 Hz, 3H).

### 2-ethyl-5-(4,4,5,5-tetramethyl-1,3,2-dioxaborolan-2-yl)-2H-indazole

Under inert atmosphere, a mixture of 5-bromo-2-ethyl-2H-indazole (0.35 g, 1.56 mmol, 1.0 eq), bis(pinacolato)diboron (0.592 g, 2.33 mmol, 1.50 eq), potassium acetate (0.305 g, 3.11 mmol, 2.00 eq), and 1,1′-bis(diphenylphosphino)ferrocene palladium (II) dichloride (0.057 g, 0.078 mmol, 0.05 eq) in anhydrous dioxane (3.5 mL) was stirred at 90° C. for 16 hours. The reaction mixture was concentrated in vacuo and diluted with ethyl acetate. The organic layer was washed with water, dried over anhydrous sodium sulfate, and concentrated. The residue was purified by silica gel column chromatography (10-30% EtOAc in hexanes) to give 2-ethyl-5-(4,4,5,5-tetramethyl-1,3,2-dioxaborolan-2-yl)-2H-indazole (0.38 g, 1.40 mmol, 80%).

LCMS (ESI+): m/z 272.95, [M+H]⁺.

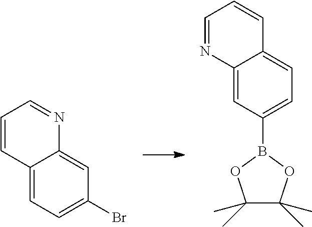 [SMILES: Brc1ccc2cccnc2c1 ; CC1(C)OB(c2ccc3cccnc3c2)OC1(C)C]

### 7-(4,4,5,5-tetramethyl-1,3,2-dioxaborolan-2-yl)quinoline

To a suspension of 4,4,4′,4′,5,5,5′,5′-octamethyl-2,2′-bi(1,3,2-dioxaborolane) (589 mg, 1.2 Eq, 779 μmol), 7-bromoquinoline (300 mg, 1 Eq, 649 μmol), and potassium acetate (382 mg, 2.8 Eq, 1.82 mmol) in DMF (5 mL) was added Pd(dppf)Cl₂·DCM (61.4 mg, 0.05 Eq, 32.5 μmol) at room temperature. The mixture was then stirred at 100° C. under nitrogen atmosphere for 2 hours. The reaction mixture was directly purified with silica gel column chromatography (0-30% EtOAc in heptane) to obtain 7-(4,4,5,5-tetramethyl-1,3,2-dioxaborolan-2-yl)quinoline (320 mg, 401 μmol, 61.8) as a yellow solid.

LCMS (ESI+): 174.2 [M+H-pinacol]⁺.

Other Compounds Prepared Similarly:

### 8-fluoro-7-(4,4,5,5-tetramethyl-1,3,2-dioxaborolan-2-yl)quinolone

LCMS (ESI+): 192.2 [M+H-pinacol]⁺.

### 8-fluoro-3-(4,4,5,5-tetramethyl-1,3,2-dioxaborolan-2-yl)quinolone

LCMS (ESI+): 192.1 [M+H-pinacol]⁺.

### (E)-1-(2-ethoxyvinyl)-6-(4,4,5,5-tetramethyl-1,3,2-dioxaborolan-2-yl)isoquinoline

LCMS (ESI+): m/z 326.1, [M+H]⁺

### 1,3-dimethyl-6-(4,4,5,5-tetramethyl-1,3,2-dioxaborolan-2-yl)isoquinoline

LCMS (ESI+): m/z 202.3, [M+H-pinacol]⁺

### 3-((1S,2R)-2-methylcyclopropyl)-6-(4,4,5,5-tetramethyl-1,3,2-dioxaborolan-2-yl)isoquinoline

LCMS (ESI+): m/z 228.2, [M+H-pinacol]⁺

### (1-41R,2R)-2-(hydroxymethyl)cyclopropyl)isoquinolin-6-yl)boronic acid

LCMS (ESI+): m/z 244.1, [M+H-pinacol]⁺

### 1-(2-methoxyethyl)-6-(4,4,5,5-tetramethyl-1,3,2-dioxaborolan-2-yl)isoquinoline

LCMS (ESI+): m/z 232.1, [M+H-pinacol]⁺

Preparation of Compound 176 Via Suzuki Coupling of Pinacol Boronate to 2-halo-oxazole with Pd(dppf)Cl₂.

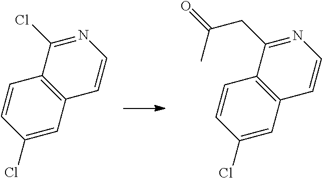

### 1-(6-chloroisoquinolin-1-yl)propan-2-one

To a suspension of 1,6-dichloroisoquinoline (2.00 g, 1 Eq, 10.1 mmol), methoxytributyltin (4.86 g, 4.33 mL, 1.5 Eq, 15.2 mmol), 2-dicyclohexylphosphino-2,6-dimethoxy-1,1-biphenyl (415 mg, 0.1 Eq, 1.01 mmol), and isopropenyl acetate (1.52 g, 1.66 mL, 1.5 Eq, 15.2 mmol) in DMF (5 mL) was added bis(dibenzylideneacetone)palladium (581 mg, 0.1 Eq, 1.01 mmol) at room temperature. The mixture was then stirred at 80° C. under nitrogen atmosphere for 2 hours. The crude reaction was concentrated in vacuo and purified by silica gel column chromatography (0-100% EtOAc in heptane) to provide 1-(6-chloroisoquinolin-1-yl)propan-2-one (1.1 g, 5.0 mmol, 50%) as a brown oil.

LCMS (ESI+): m/z 220.1, [M+H]⁺

### 1-(6-(4,4,5,5-tetramethyl-1,3,2-dioxaborolan-2-yl)isoquinolin-1-yl)propan-2-one

Prepared from 1-(6-chloroisoquinolin-1-yl)propan-2-one via borylation procedure above.

LCMS (ESI+): m/z 312.2, [M+H]⁺

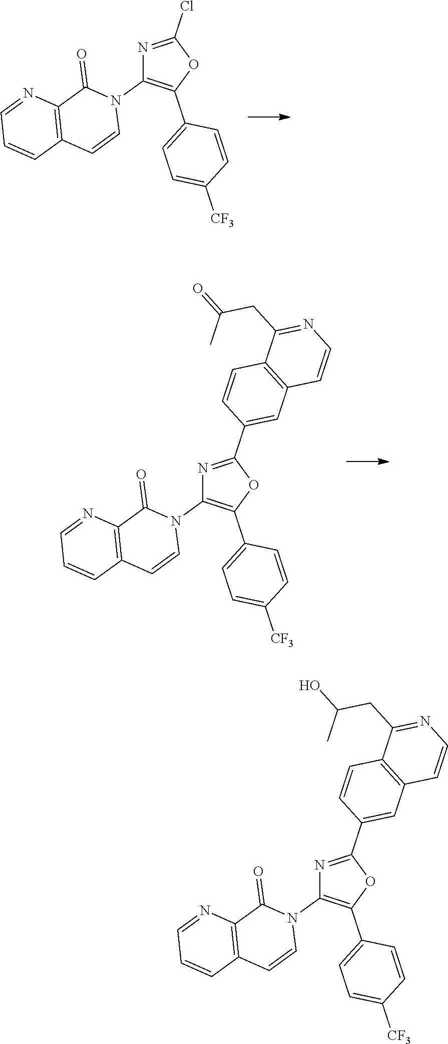 [SMILES: O=c1c2ncccc2ccn1-c1nc(Cl)oc1-c1ccc(C(F)(F)F)cc1 ; CC(=O)Cc1nccc2cc(-c3nc(-n4ccc5cccnc5c4=O)c(-c4ccc(C(F)(F)F)cc4)o3)ccc12 ; CC(O)Cc1nccc2cc(-c3nc(-n4ccc5cccnc5c4=O)c(-c4ccc(C(F)(F)F)cc4)o3)ccc12]

### 7-(2-(1-(2-oxopropyl)isoquinolin-6-yl)-5-(4-(trifluoromethyl)phenyl)oxazol-4-yl)-1,7-naphthyridin-8(7H)-one

A mixture of 1-(6-(4,4,5,5-tetramethyl-1,3,2-dioxaborolan-2-yl)isoquinolin-1-yl)propan-2-one (143 mg, 1.5 Eq, 460 μmol), 7-(2-chloro-5-(4-(trifluoromethyl)phenyl)oxazol-4-yl)-1,7-naphthyridin-8(7H)-one (120 mg, 1 Eq, 306 μmol), Pd(dppf)Cl₂ (22.4 mg, 0.1 Eq, 30.6 μmol), and cesium carbonate (230 mg, 2.3 Eq, 705 μmol) in 1,4-dioxane (1.3 mL) and water (0.2 mL) was stirred at 90° C. for 1.5 hours. The reaction mixture was cooled to room temperature and poured into water. The mixture was extracted with DCM, and the combined organic extracts were dried over sodium sulfate and concentrated. The material was mixed with 4 M HCl in dioxane and stirred for 30 minutes. At this time, the mixture was loaded onto silica gel and purified by silica gel column chromatography using (0-10% MeOH in DCM). This provided 7-(2-(1-(2-oxopropyl)isoquinolin-6-yl)-5-(4-(trifluoromethyl)phenyl)oxazol-4-yl)-1,7-naphthyridin-8 (7H)-one (50 mg, 93 μmol, 30%) as an orange solid.

LCMS (ESI+): m/z 541.1, [M+H]⁺

### 7-(2-(1-(2-oxopropyl)isoquinolin-6-yl)-5-(4-(trifluoromethyl)phenyl)oxazol-4-yl)-1,7-naphthyridin-8(7H)-one. Compound 468

A mixture of 7-(2-(1-(2-oxopropyl)isoquinolin-6-yl)-5-(4-(trifluoromethyl)phenyl)oxazol-4-yl)-1,7-naphthyridin-8(7H)-one (50 mg, 1 Eq, 93 μmol) and sodium borohydride (10 mg, 3 Eq, 0.28 mmol) in 9:1 THF/MeOH (2 mL) was stirred at room temperature for 3 hours. At this time, the crude reaction was loaded onto silica gel and purified by silica gel column chromatography (0-10% MeOH in DCM). This provided 7-(2-(1-(2-oxopropyl)isoquinolin-6-yl)-5-(4-(trifluoromethyl)phenyl)oxazol-4-yl)-1,7-naphthyridin-8(7H)-one as a white solid.

LCMS (ESI+): m/z 543.0, [M+H]⁺

1H NMR (400 MHz, DMSO) δ 8.90 (d, J=4.4 Hz, 1H), 8.87 (s, 1H), 8.54 (t, J=8.2 Hz, 2H), 8.34 (d, J=9.0 Hz, 1H), 8.29 (d, J=8.1 Hz, 1H), 7.93 (d, J=5.7 Hz, 1H), 7.90-7.83 (m, 5H), 7.76 (d, J=7.4 Hz, 1H), 6.92 (d, J=7.4 Hz, 1H), 4.83 (d, J=4.6 Hz, 1H), 4.25 (m, 1H), 3.48 (dd, J=13.8, 7.1 Hz, 1H), 3.26 (m, 1H), 1.19 (d, J=6.1 Hz, 3H).

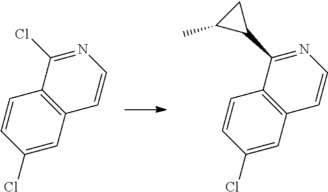

### (rac)-6-chloro-1-((trans)-2-methylcyclopropyl)isoquinoline

1,6-Dichloroisoquinoline (1.00 g, 1 Eq, 5.05 mmol), trans-2-methyl-cyclopropyl boronic acid pinacol ester (2.30 g, 2.5 Eq, 12.6 mmol), potassium phosphate tribasic (2.68 g, 2.5 Eq, 12.6 mmol), and cesium carbonate (3.29 g, 2 Eq, 10.1 mmol) were dissolved in 1,4-dioxane (5 mL), and the mixture was sparged with argon for five minutes. At this time, Pd(dppf)Cl₂·DCM (185 mg, 0.05 Eq, 253 μmol) was added, and the reaction mixture was heated to 90° C. for 4 hours. The reaction mixture was cooled to room temperature, diluted with DCM, washed with water, concentrated, and purified by silica gel column chromatography (35% EtOAc in heptane) to give (rac)-6-chloro-1-((trans)-2-methylcyclopropyl)isoquinoline (405 mg, 1.86 mmol, 36.8%) as a yellow crystalline solid.

LCMS (ESI+): m/z 218.1, [M+H]⁺

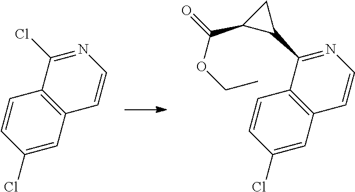

### (rac)-(cis)-2-(6-chloroisoquinolin-1-yl)cyclopropane-1

1,6-Dichloroisoquinoline (4.00 g, 1 Eq, 20.2 mmol), ethyl 2-(4,4,5,5-tetramethyl-1,3,2-dioxaborolan-2-yl)cyclopropane-1-carboxylate (5.33 g, 1.1 Eq, 22.2 mmol), potassium phosphate tribasic (10.7 g, 2.5 Eq, 50.5 mmol), and 1,1′-bis(diphenylphosphino)ferrocene-palladium(II) dichloride (1.48 g, 0.1 Eq, 2.02 mmol) were dissolved in 1,4-dioxane (5 mL), and the reaction mixture was sparged with argon for five minutes. The reaction mixture was then heated to 90° C. for 16 hours. LCMS shows two products or equal mass indicating the presence of both diastereomers. The reaction mixture was cooled to room temperature, diluted with DCM, washed with water, concentrated, and purified by normal phase column chromatography (65% EtOAc in heptane) to give ethyl (rac)-(trans)-2-(6-chloroisoquinolin-1-yl)cyclopropane-1-carboxylate (1.1 g, 4.0 mmol, 20%) and ethyl (rac)-(cis)-2-(6-chloroisoquinolin-1-yl)cyclopropane-1-carboxylate (1.5 g, 5.4 mmol, 27%) as a brown oil.

LCMS (ESI+): m/z 276.0, [M+H]⁺

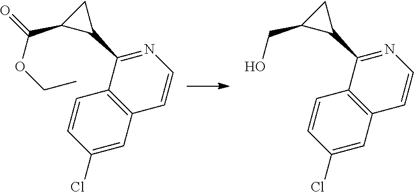

### (rac)-((cis)-2-(6-chloroisoquinolin-1-yl)cyclopropyl)methanol

Ethyl (rac)-(cis)-2-(6-chloroisoquinolin-1-yl)cyclopropane-1-carboxylate (1.1 g, 1 Eq, 4.0 mmol) was dissolved in THF (5 mL). Lithium aluminum hydride (0.18 g, 1.2 Eq, 4.8 mmol) was added, and the mixture was allowed to stir at room temperature for 3 hours. Upon completion, a 1 M solution of Rochelle's salt was added, and the mixture was stirred for 1 hour. This mixture was then extracted with DCM, and the combined organic layers were dried and concentrated. This material was purified by normal phase column chromatography (0-10% MeOH in DCM) to yield (rac)-((cis)-2-(6-chloroisoquinolin-1-yl)cyclopropyl)methanol (122 mg, 522 μmol, 13%) as a red oil

LCMS (ESI+): m/z 234.1, [M+H]⁺

### (rac)-3-((cis)-2-methylcyclopropyl)-6-(4,4,5,5-tetramethyl-1,3,2-dioxaborolan-2-yl)isoquinoline

Prepared via borylation described above.

LCMS (ESI+): m/z 228.2, [M+H-pinacol]⁺

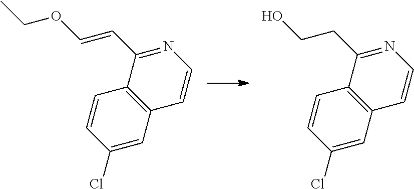 [SMILES: CCO/C=C/c1nccc2cc(Cl)ccc12 ; OCCc1nccc2cc(Cl)ccc12]

### 2-(6-chloroisoquinolin-1-yl)ethan-1-ol

(E)-6-Chloro-1-(2-ethoxyvinyl)isoquinoline (292 mg, 1 Eq, 1.25 mmol) was dissolved in 1,4-dioxane (1 mL). Aqueous hydrogen chloride (456 mg, 1.04 mL, 12 molar, 10 Eq, 12.5 mmol) was added dropwise. The reaction was heated to 80° C. for 16 hours. The reaction was cooled to room temperature and quenched with aqueous sodium bicarbonate. The crude mixture was then extracted with DCM, and the combined organic layers were dried and concentrated. The resulting residue was purified by normal phase column chromatography (10% MeOH in DCM) to yield a yellow solid intermediate 2-(6-chloroisoquinolin-1-yl)acetaldehyde. 2-(6-chloroisoquinolin-1-yl)acetaldehyde was dissolved in MeOH (1 mL) and sodium borohydride (142 mg, 3 Eq, 3.75 mmol) was added. The mixture was stirred for 1 hour at room temperature. The reaction was then quenched with aqueous ammonium chloride and extracted with DCM. The combined organic solution was concentrated to obtain 2-(6-chloroisoquionlin-1-yl)ethan-1-ol (92 mg, 0.44 mmol, 35%) as a brown solid.

LCMS (ESI+): m/z 208.1, [M+H]⁺

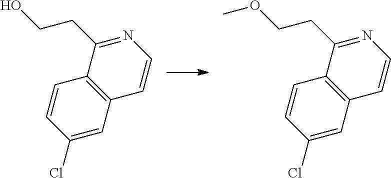

### 6-chloro-1-(2-methoxyethyl)isoquinoline

2-(6-Chloroisoquinolin-1-yl)ethan-1-ol (463 mg, 1 Eq, 2.23 mmol), p-toluenesulfonyl chloride (1.28 g, 3 Eq, 6.69 mmol), Hunig's Base (865 mg, 1.17 mL, 3 Eq, 6.69 mmol), and DMAP (136 mg, 0.5 Eq, 1.11 mmol) were combined with DCM (5 mL), and the reaction mixture was stirred at room temperature for 16 hours. The crude reaction was washed with aqueous ammonium chloride, and the mixture was separated. The organic solution was concentrated under reduced pressure to give a crude material. To this material was added methanol (5 mL), and the solution was heated in the microwave at 100° C. for 1 hour. The reaction was cooled and concentrated under reduced pressure to give 6-chloro-1-(2-methoxyethyl)isoquinoline (301 mg, 1.36 mmol, 60.9%) as an orange foam.

LCMS (ESI+): m/z 222.1, [M+H]⁺

Additional Compounds were Prepared in an Analogous Manner to Compound 468, Via Suzuki Coupling of Pinacol Boronate to 2-halo-oxazole Intermediates with Pd(dppf)Cl₂:

| | Exact mass | Observed | LCMS |
|---|---|---|---|
| Compound | (g/mol) | m/z(+) | Assignment |
| 469 | 397.1227 | 398.1 | [M + H]+ |
| 470 | 399.0775 | 400.0 | [M + H]+ |
| 471 | 401.0976 | 402.0 | [M + H]+ |
| 472 | 411.1383 | 412.1 | [M + H]+ |
| 473 | 412.1336 | 413.1 | [M + H]+ |
| 474 | 413.0931 | 414.0 | [M + H]+ |
| 475 | 415.1132 | 416.0 | [M + H]+ |
| 476 | 415.1132 | 416.0 | [M + H]+ |
| 477 | 417.068 | 418.0 | [M + H]+ |
| 478 | 419.0882 | 420.0 | [M + H]+ |
| 479 | 424.0972 | 425.0 | [M + H]+ |
| 480 | 425.1176 | 426.1 | [M + H]+ |
| 481 | 425.1176 | 426.1 | [M + H]+ |
| 482 | 425.1176 | 426.0 | [M + H]+ |
| 483 | 426.0928 | 427.0 | [M + H]+ |
| 484 | 427.1445 | 428.1 | [M + H]+ |
| 485 | 428.104 | 429.0 | [M + H]+ |
| 486 | 431.1081 | 432.0 | [M + H]+ |
| 487 | 431.1081 | 432.1 | [M + H]+ |
| 488 | 432.0789 | 433.1 | [M + H]+ |
| 489 | 433.0385 | 473.1 | [M + H]+ |
| 490 | 433.0385 | 433.7 | [M + H]+ |
| 491 | 433.1227 | 434.0 | [M + H]+ |
| 492 | 434.1179 | 435.0 | [M + H]+ |
| 493 | 434.1179 | 435.0 | [M + H]+ |
| 494 | 434.1179 | 435.0 | [M + H]+ |
| 495 | 434.1179 | 435.0 | [M + H]+ |
| 496 | 434.1179 | 435.0 | [M + H]+ |
| 497 | 435.0586 | 436.0 | [M + H]+ |
| 498 | 435.0586 | 436.0 | [M + H]+ |
| 499 | 436.1336 | 437.0 | [M + H]+ |
| 500 | 437.1176 | 438.0 | [M + H]+ |
| 501 | 437.1288 | 438.0 | [M + H]+ |
| 502 | 437.1288 | 438.0 | [M + H]+ |
| 503 | 437.1288 | 438.1 | [M + H]+ |
| 504 | 438.1128 | 439.0 | [M + H]+ |
| 505 | 438.1128 | 439.0 | [M + H]+ |
| 506 | 440.0676 | 441.0 | [M + H]+ |
| 507 | 441.0925 | 442.0 | [M + H]+ |
| 508 | 441.1125 | 442.0 | [M + H]+ |
| 509 | 443.1081 | 444.0 | [M + H]+ |
| 510 | 447.0786 | 448.0 | [M + H]+ |
| 511 | 447.1132 | 448.0 | [M + H]+ |
| 512 | 447.1195 | 448.0 | [M + H]+ |
| 513 | 447.1195 | 448.3 | [M + H]+ |
| 514 | 448.1336 | 449.0 | [M + H]+ |
| 515 | 449.0931 | 450.1 | [M + H]+ |
| 516 | 449.0987 | 450.0 | [M + H]+ |
| 517 | 450.0884 | 451.0 | [M + H]+ |
| 518 | 451.0944 | 452.0 | [M + H]+ |
| 519 | 451.0944 | 452.1 | [M + H]+ |
| 520 | 451.1144 | 452.1 | [M + H]+ |
| 521 | 452.0896 | 453.1 | [M + H]+ |
| 522 | 452.1085 | 453.0 | [M + H]+ |
| 523 | 452.1085 | 453.0 | [M + H]+ |
| 524 | 452.1285 | 453.1 | [M + H]+ |
| 525 | 452.1285 | 453.3 | [M + H]+ |
| 526 | 453.0993 | 454.1 | [M + H]+ |
| 527 | 455.1005 | 456.0 | [M + H]+ |
| 528 | 455.1194 | 456.1 | [M + H]+ |
| 529 | 455.1194 | 456.0 | [M + H]+ |
| 530 | 455.1194 | 456.0 | [M + H]+ |
| 531 | 458.0991 | 459.1 | [M + H]+ |
| 532 | 462.1304 | 463.1 | [M + H]+ |
| 533 | 462.1304 | 463.1 | [M + H]+ |
| 534 | 463.1144 | 464.1 | [M + H]+ |
| 535 | 463.1144 | 464.1 | [M + H]+ |
| 536 | 464.1285 | 465.1 | [M + H]+ |
| 537 | 465.11 | 465.8 | [M + H]+ |
| 538 | 467.0648 | 468.0 | [M + H]+ |
| 539 | 467.0648 | 468.0 | [M + H]+ |
| 540 | 467.0648 | 468.1 | [M + H]+ |
| 541 | 474.094 | 475.0 | [M + H]+ |
| 542 | 474.094 | 475.1 | [M + H]+ |
| 543 | 474.1304 | 474.8 | [M + H]+ |
| 544 | 474.1304 | 475.1 | [M + H]+ |
| 545 | 475.1144 | 476.1 | [M + H]+ |
| 546 | 475.1144 | 476.1 | [M + H]+ |
| 547 | 475.1144 | 476.1 | [M + H]+ |
| 548 | 476.146 | 477.2 | [M + H]+ |
| 549 | 476.146 | 477.1 | [M + H]+ |
| 550 | 478.1253 | 479.0 | [M + H]+ |
| 552 | 482.0757 | 483.0 | [M + H]+ |
| 553 | 484.1147 | 484.9 | [M + H]+ |
| 554 | 484.1147 | 484.8 | [M + H]+ |
| 555 | 484.1147 | 485.1 | [M + H]+ |
| 556 | 485.0554 | 486.0 | [M + H]+ |
| 557 | 485.0554 | 486.0 | [M + H]+ |
| 558 | 485.0799 | 486.0 | [M + H]+ |
| 559 | 486.1304 | 487.1 | [M + H]+ |
| 560 | 487.1144 | 487.9 | [M + H]+ |
| 561 | 487.1144 | 488.0 | [M + H]+ |
| 562 | 487.1256 | 488.1 | [M + H]+ |
| 563 | 487.1256 | 488.1 | [M + H]+ |
| 564 | 487.1256 | 488.1 | [M + H]+ |
| 565 | 488.1096 | 489.0 | [M + H]+ |
| 566 | 489.0759 | 490.0 | [M + H]+ |
| 567 | 491.0893 | 492.1 | [M + H]+ |
| 568 | 492.1045 | 493.2 | [M + H]+ |
| 569 | 495.177 | 496.1 | [M + H]+ |
| 570 | 497.0754 | 498.0 | [M + H]+ |
| 571 | 497.11 | 497.8 | [M + H]+ |
| 572 | 498.0707 | 499.0 | [M + H]+ |
| 573 | 498.1304 | 498.9 | [M + H]+ |
| 574 | 498.1304 | 498.9 | [M + H]+ |
| 575 | 498.1304 | 499.2 | [M + H]+ |
| 576 | 498.1304 | 499.1 | [M + H]+ |
| 577 | 498.1304 | 499.1 | [M + H]+ |
| 578 | 499.0955 | 500.1 | [M + H]+ |
| 579 | 499.1256 | 499.9 | [M + H]+ |
| 580 | 501.0259 | 501.9 | [M + H]+ |
| 581 | 501.1413 | 502.0 | [M + H]+ |
| 582 | 501.1413 | 502.0 | [M + H]+ |
| 583 | 501.1413 | 501.8 | [M + H]+ |
| 584 | 501.1413 | 502.5 | [M + H]+ |
| 585 | 501.1413 | 502.2 | [M + H]+ |
| 586 | 502.1053 | 502.9 | [M + H]+ |
| 587 | 502.1053 | 503.2 | [M + H]+ |
| 588 | 502.1053 | 503.0 | [M + H]+ |
| 589 | 502.1053 | 503.0 | [M + H]+ |
| 590 | 502.1253 | 503.0 | [M + H]+ |
| 591 | 502.1253 | 503.1 | [M + H]+ |
| 592 | 503.1093 | 504.1 | [M + H]+ |
| 593 | 503.1093 | 504.1 | [M + H]+ |
| 594 | 504.1045 | 505.3 | [M + H]+ |
| 595 | 504.1409 | 505.1 | [M + H]+ |
| 596 | 505.1162 | 505.9 | [M + H]+ |
| 597 | 505.1162 | 505.9 | [M + H]+ |
| 598 | 505.1162 | 506.0 | [M + H]+ |
| 599 | 512.0863 | 512.8 | [M + H]+ |
| 600 | 512.1209 | 512.8 | [M + H]+ |
| 601 | 512.1209 | 513.2 | [M + H]+ |
| 602 | 512.146 | 513.0 | [M + H]+ |
| 603 | 513.1413 | 514.0 | [M + H]+ |
| 604 | 513.1413 | 514.0 | [M + H]+ |
| 605 | 514.1253 | 514.9 | [M + H]+ |
| 606 | 514.1253 | 514.9 | [M + H]+ |
| 607 | 514.1253 | 515.2 | [M + H]+ |
| 608 | 515.1569 | 516.0 | [M + H]+ |
| 609 | 515.1569 | 516.1 | [M + H]+ |
| 610 | 515.1569 | 515.9 | [M + H]+ |
| 611 | 515.1569 | 516.2 | [M + H]+ |
| 612 | 516.1209 | 516.9 | [M + H]+ |
| 613 | 516.1209 | 517.2 | [M + H]+ |
| 614 | 516.1409 | 517.1 | [M + H]+ |
| 615 | 516.1409 | 516.9 | [M + H]+ |
| 616 | 516.1409 | 517.2 | [M + H]+ |
| 617 | 517.0861 | 518.1 | [M + H]+ |
| 618 | 517.0861 | 518.1 | [M + H]+ |
| 619 | 517.1362 | 518.1 | [M + H]+ |
| 620 | 517.1362 | 517.6 | [M + H]+ |
| 621 | 517.1362 | 518.2 | [M + H]+ |
| 622 | 518.0757 | 519.1 | [M + H]+ |
| 623 | 518.0757 | 519.1 | [M + H]+ |
| 624 | 518.0757 | 519.1 | [M + H]+ |
| 625 | 518.0757 | 519.0 | [M + H]+ |
| 626 | 518.1202 | 519.1 | [M + H]+ |
| 627 | 519.1318 | 519.9 | [M + H]+ |
| 628 | 519.1318 | 520.2 | [M + H]+ |
| 629 | 521.0866 | 522.0 | [M + H]+ |
| 630 | 521.0866 | 522.0 | [M + H]+ |
| 631 | 521.0866 | 522.2 | [M + H]+ |
| 632 | 523.1068 | 523.9 | [M + H]+ |
| 633 | 523.1068 | 524.1 | [M + H]+ |
| 634 | 527.1569 | 528.0 | [M + H]+ |
| 635 | 527.1569 | 528.0 | [M + H]+ |
| 636 | 527.1569 | 528.0 | [M + H]+ |
| 637 | 527.1569 | 528.1 | [M + H]+ |
| 638 | 528.1409 | 529.0 | [M + H]+ |
| 639 | 528.1409 | 529.1 | [M + H]+ |
| 640 | 529.0719 | 530.0 | [M + H]+ |
| 641 | 529.1362 | 530.2 | [M + H]+ |
| 642 | 530.1566 | 531.5 | [M + H]+ |
| 643 | 532.1159 | 533.2 | [M + H]+ |
| 644 | 532.1159 | 532.8 | [M + H]+ |
| 645 | 537.1224 | 537.9 | [M + H]+ |
| 646 | 537.1224 | 538.4 | [M + H]+ |
| 647 | 538.1617 | 539.2 | [M + H]+ |
| 648 | 541.1726 | 542.0 | [M + H]+ |
| 649 | 542.1566 | 543.0 | [M + H]+ |
| 650 | 544.1722 | 545.0 | [M + H]+ |
| 651 | 554.1566 | 555.2 | [M + H]+ |
| 652 | 554.1566 | 555.2 | [M + H]+ |
| 653 | 555.113 | 555.8 | [M + H]+ |
| 654 | 555.113 | 555.9 | [M + H]+ |
| 655 | 568.1722 | 569.0 | [M + H]+ |
| 656 | 579.1188 | 580.2 | [M + H]+ |
| 657 | 585.1236 | 585.9 | [M + H]+ |

Preparation of Compound 551 Via Borylation and Suzuki Coupling of Pinacol Boronate to 2-halo-oxazole.

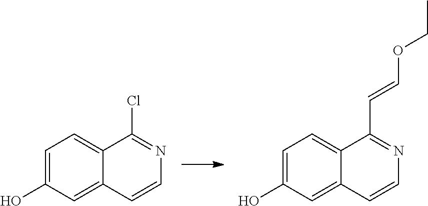 [SMILES: Oc1ccc2c(Cl)nccc2c1 ; CCO/C=C/c1nccc2cc(O)ccc12]

### (E)-1-(2-ethoxyvinyl)isoquinolin-6-ol

1-Chloroisoquinolin-6-ol (2.00 g, 1 Eq, 11.1 mmol), (Z)-2-(2-ethoxyvinyl)-4,4,5,5-tetramethyl-1,3,2-dioxaborolane (3.31 g, 1.5 Eq, 16.7 mmol), Pd(dppf) (909 mg, 0.1 Eq, 1.11 mmol), and cesium carbonate (5.44 g, 1.5 Eq, 16.7 mmol) were dissolved in dioxane (50 mL) and water (5.0 mL), and the mixture was sparged with argon for five minutes. The reaction mixture was then heated to 85° C. for 4 hours. The reaction mixture was cooled to room temperature and diluted with DCM and water. The layers were separated, and the organic layer was concentrated to give a crude material. This material was purified by silica gel column chromatography (65% EtOAc in heptane) to give (E)-1-(2-ethoxyvinyl)isoquinolin-6-ol (990 mg, 4.60 mmol, 41.3%) as a yellow crystalline solid.

LCMS (ESI+): 216.2 [M+H]⁺.

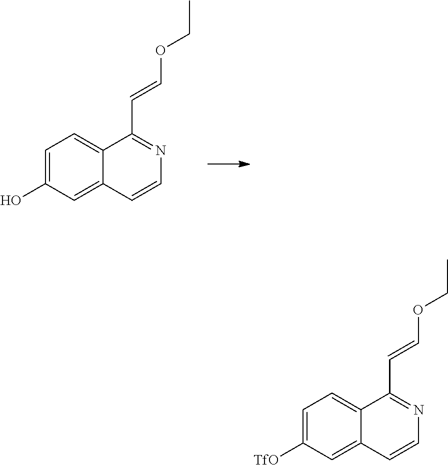 [SMILES: CCO/C=C/c1nccc2cc(O)ccc12 ; CCO/C=C/c1nccc2cc(OS(=O)(=O)C(F)(F)F)ccc12]

### (E)-1-(2-ethoxyvinyl)isoquinolin-6-yl trifluoromethanesulfonate

(E)-1-(2-Ethoxyvinyl)isoquinolin-6-ol (163 mg, 1 Eq, 757 μmol) and sodium hydride (60.6 mg, 60 Wt % in mineral oil, 2.00 Eq, 1.52 mmol) were dissolved in DMF (20 mL). After stirring at 50° C. for 20 min, phenyl triflimide (325 mg, 1.2 Eq, 909 μmol) was added, and the mixture was stirred at room temperature for 30 minutes. The reaction was then quenched with 1 mL of water and the solvent was evaporated in vacuo. The resulting residue was redissolved in DCM and washed with water. The organic solution was dried over Na₂SO₄, filtered, and concentrated to afford the crude product. This material was purified by silica gel chromatography (0-10% MeOH in DCM) to afford (E)-1-(2-ethoxyvinyl)isoquinolin-6-yl trifluoromethanesulfonate (384 mg, 1.11 mmol, 146%) as a green impure solid.

LCMS (ESI+): 348.0 [M+H]⁺.

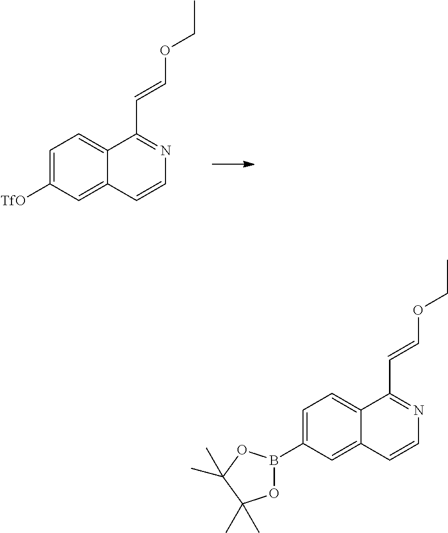 [SMILES: CCO/C=C/c1nccc2cc(OS(=O)(=O)C(F)(F)F)ccc12 ; CCO/C=C/c1nccc2cc(B3OC(C)(C)C(C)(C)O3)ccc12]

### (E)-1-(2-ethoxyvinyl)-6-(4,4,5,5-tetramethyl-1,3,2-dioxaborolan-2-yl)isoquinoline

A mixture of N-4,4,4′,4′,5,5,5′,5′-octamethyl-2,2′-bi(1,3,2-dioxaborolane) (337 mg, 1.2 Eq, 1.33 mmol), (E)-1-(2-ethoxyvinyl)isoquinolin-6-yl trifluoromethanesulfonate (384 mg, 1 Eq, 1.11 mmol), potassium acetate (304 mg, 2.8 Eq, 3.10 mmol), and bis-(triphenylphosphino)-palladium chloride (38.8 mg, 0.05 Eq, 55.3 μmol) in DMF (5 mL) was heated to 90° C. for 16 hours. The mixture was cooled to rt and concentrated in vacuo, and the resulting residue was diluted with DCM. The resulting mixture was washed with water (20 mL) and saturated aqueous NaCl, dried over anhydrous Na₂SO₄, and concentrated in vacuo. The residue was purified by silica gel column chromatography (0-10% MeOH in DCM) to give (E)-1-(2-ethoxyvinyl)-6-(4,4,5,5-tetramethyl-1,3,2-dioxaborolan-2-yl)isoquinoline (140 mg, 430 μmol, 38.9%) as a brown solid.

LCMS (ESI+): 244.3 [M+H-pinacol]⁺.

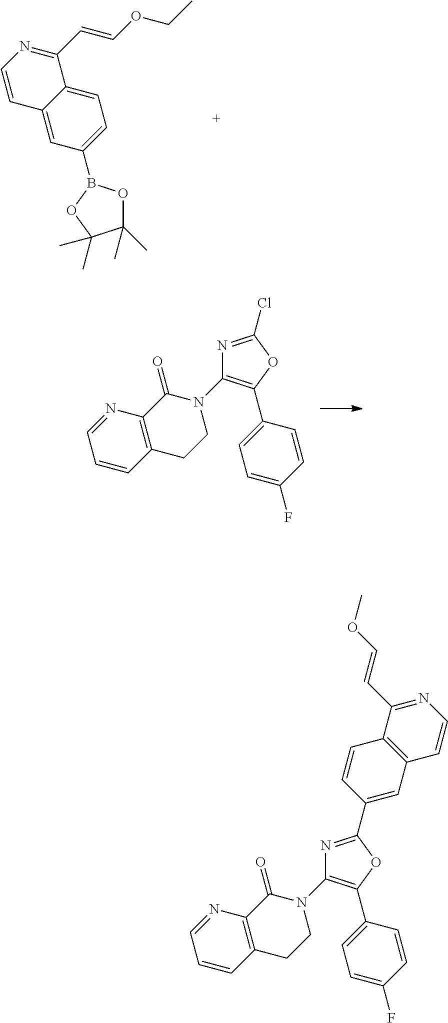 [SMILES: CCO/C=C/c1nccc2cc(B3OC(C)(C)C(C)(C)O3)ccc12 ; O=C1c2ncccc2CCN1c1nc(Cl)oc1-c1ccc(F)cc1 ; CO/C=C/c1nccc2cc(-c3nc(N4CCc5cccnc5C4=O)c(-c4ccc(F)cc4)o3)ccc12]

### (E)-7-(2-(1-(2-ethoxyvinyl)isoquinolin-6-yl)-5-(4-fluorophenyl)oxazol-4-yl)-1,7-naphthyridin-8(7H)-one

A mixture of (E)-1-(2-ethoxyvinyl)-6-(4,4,5,5-tetramethyl-1,3,2-dioxaborolan-2-yl)isoquinoline (87 mg, 1.3 Eq, 0.27 mmol), 7-(2-chloro-5-(4-fluorophenyl)oxazol-4-yl)-1,7-naphthyridin-8(7H)-one (70 mg, 1 Eq, 0.20 mmol), Pd(dppf) (15 mg, 0.1 Eq, 20 μmol), and cesium carbonate (0.15 g, 2.3 Eq, 0.47 mmol) in 1,4-Dioxane (1.3 mL) and water (0.2 mL) was stirred at 90° C. for 1.5 hours. The reaction mixture was cooled to room temperature and concentrated onto silica gel. This material was purified by silica gel column chromatography (0-10% MeOH in DCM). This purification provided (E)-7-(2-(1-(2-ethoxyvinyl)isoquinolin-6-yl)-5-(4-fluorophenyl)oxazol-4-yl)-1,7-naphthyridin-8(7H)-one (35 mg, 69 μmol, 34%) as a white solid.

LCMS (ESI+): 505.1 [M+H]⁺.

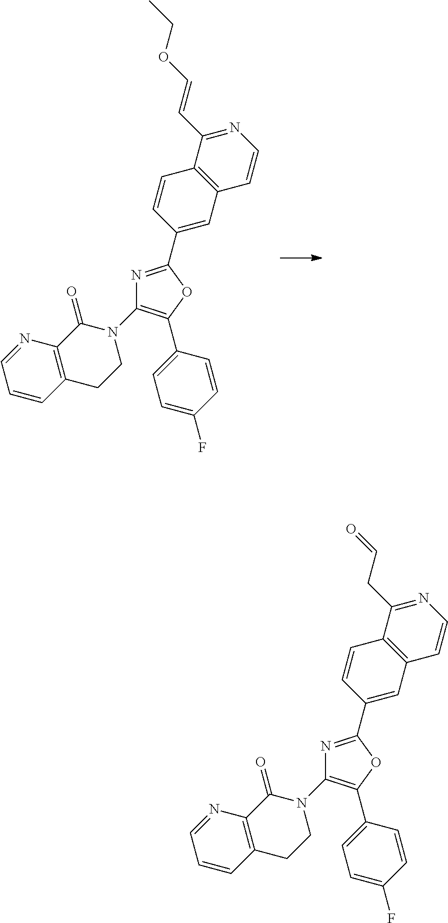 [SMILES: CCO/C=C/c1nccc2cc(-c3nc(N4CCc5cccnc5C4=O)c(-c4ccc(F)cc4)o3)ccc12 ; O=CCc1nccc2cc(-c3nc(N4CCc5cccnc5C4=O)c(-c4ccc(F)cc4)o3)ccc12]

### 7-(2-(1-chloroisoquinolin-6-yl)-5-(4-fluorophenyl)oxazol-4-yl)-1,7-naphthyridin-8(7H)-one

To aqueous HCl (12 M, 0.06 mL, 0.72 mmol, 4.8 Eq) in 1,4-dioxane (2 mL) was added

(E)-7-(2-(1-(2-ethoxyvinyl)isoquinolin-6-yl)-5-(4-fluorophenyl)oxazol-4-yl)-1,7-naphthyridin-8(7H)-one (75 mg, 1 Eq, 0.15 mmol) and the resulting mixture was heated to reflux for 2.5 hours. The mixture was cooled to room temperature and diluted with ethyl acetate (100 mL). The organic layer was washed with brine (4×100 mL), dried over sodium sulfate, filtered, and concentrated under reduced pressure to afford 2-(6-(5-(4-fluorophenyl)-4-(8-oxo-1,7-naphthyridin-7(8H)-yl)oxazol-2-yl)isoquinolin-1-yl)acetaldehyde (35 mg, 73 μmol, 49%) as a yellow solid.

LCMS (ESI+): m/z 477.0, [M+H]⁺.

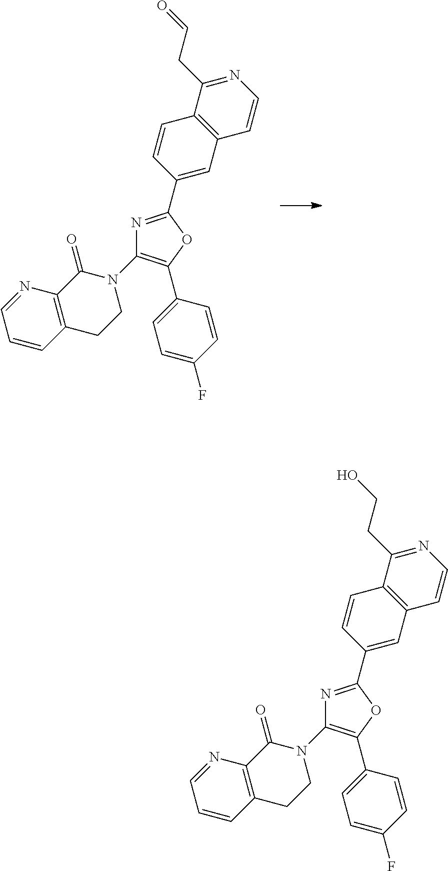 [SMILES: O=CCc1nccc2cc(-c3nc(N4CCc5cccnc5C4=O)c(-c4ccc(F)cc4)o3)ccc12 ; O=C1c2ncccc2CCN1c1nc(-c2ccc3c(CCO)nccc3c2)oc1-c1ccc(F)cc1]

### 7-(5-(4-fluorophenyl)-2-(1-(2-hydroxyethyl)isoquinolin-6-yl)oxazol-4-yl)-1,7-naphthyridin-8(7H)-one. Compound 551

Sodium borohydride (18 mg, 3 Eq, 0.47 mmol) was added to a solution of 2-(6-(5-(4-fluorophenyl)-4-(8-oxo-1,7-naphthyridin-7(8H)-yl)oxazol-2-yl)isoquinolin-1-yl)acetaldehyde (75 mg, 1 Eq, 0.16 mmol) in DMF (2 mL) and MeOH (0.2 mL), and the reaction mixture was stirred at room temperature for 1 hour. The crude mixture was treated with aqueous ammonium chloride and extracted with EtOAc. The combined organic layers were washed with brine, dried over sodium sulfate, filtered, and concentrated in vacuo. The resulting material was purified by normal phase column chromatography (0-10% MeOH in DCM) to give 7-(5-(4-fluorophenyl)-2-(1-(2-hydroxyethyl)isoquinolin-6-yl)oxazol-4-yl)-1,7-naphthyridin-8(7H)-one (13 mg, 27 μmol, 17%) as a white solid.

LCMS (ESI+): m/z 479.1, [M+H]⁺.

1H NMR (400 MHz, DMSO) δ=8.89 (d, J=4.4 Hz, 1H), 8.83 (s, 1H), 8.53 (d, J=5.7 Hz, 1H), 8.49 (d, J=8.9 Hz, 1H), 8.33 (d, J=8.9 Hz, 1H), 8.28 (d, J=8.1 Hz, 1H), 7.91 (d, J=5.8 Hz, 1H), 7.84 (dd, J=8.1, 4.5 Hz, 1H), 7.77-7.64 (m, 4H), 7.38 (t, J=8.7 Hz, 2H), 6.89 (d, J=7.4 Hz, 1H), 4.77 (t, J=5.4 Hz, 1H), 3.94 (q, J=6.4 Hz, 2H), 3.48 (t, J=6.8 Hz, 2H).

Preparation of Compound 658 Via SNAr of Aliphatic Amine with 2-chloro-oxazole Intermediate.

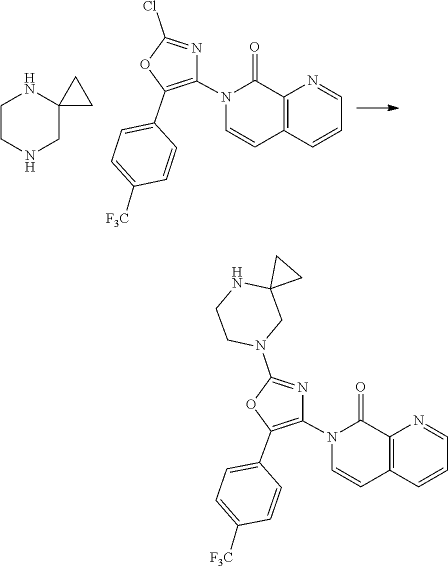

### 7-(2-(4,7-diazaspiro[2.5]octan-7-yl)-5-(4-(trifluoromethyl)phenyl)oxazol-4-yl)-1,7-naphthyridin-8(7H)-one. Compound 658

4,7-Diazaspiro[2.5]octane·2HCl (28 mg, 1.2 Eq, 0.15 mmol) and triethylamine (52 mg, 71 μL, 4 Eq, 0.51 mmol) were dissolved in DMF (1 mL). 7-(2-Chloro-5-(4-(trifluoromethyl)phenyl)oxazol-4-yl)-1,7-naphthyridin-8(7H)-one (50 mg, 1 Eq, 0.13 mmol) was added to the reaction mixture and it was allowed to stir at 60° C. for 1 hour. The crude reaction was cooled to room temperature and then absorbed onto silica gel. This material was then purified by silica gel flash chromatography (0-10% MeOH in DCM) to provide 7-(2-(4,7-diazaspiro[2.5]octan-7-yl)-5-(4-(trifluoromethyl)phenyl)oxazol-4-yl)-1,7-naphthyridin-8(7H)-one (12 mg, 26 μmol, 20%).

LCMS (ESI+): m/z 468.1, [M+H]⁺.

¹H NMR (400 MHz, DMSO-d6) d ppm 0.48-0.54 (m, 2H) 0.55-0.61 (m, 2H) 2.84-2.90 (m, 2H) 3.43 (s, 1H) 3.50-3.57 (m, 2H) 6.80 (d, J=7.34 Hz, 1H) 7.43 (d, J=8.31 Hz, 2H) 7.58 (d, J=7.34 Hz, 1H) 7.69 (d, J=8.31 Hz, 2H) 7.80 (dd, J=8.07, 4.40 Hz, 1H) 8.24 (d, J=8.07 Hz, 1H) 8.85 (d, J=4.40 Hz, 1H).

Preparation of Compound 659 Via SNAr of Nitrogen-Containing Heterocycle with 2-halo-oxazole.

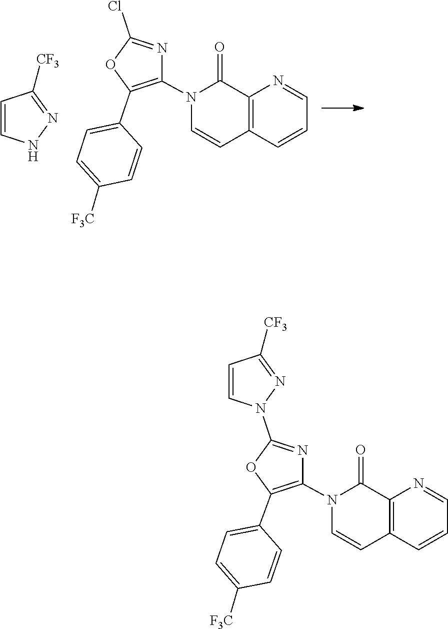

### 7-(2-(3-(trifluoromethyl)-1H-pyrazol-1-yl)-5-(4-(trifluoromethyl)phenyl)oxazol-4-yl)-1,7-naphthyridin-8(7H)-one. Compound 659

7-(2-Chloro-5-(4-(trifluoromethyl)phenyl)oxazol-4-yl)-1,7-naphthyridin-8(7H)-one (50 mg, 1 Eq, 0.13 mmol) and 5-trifluoromethyl-1H-pyrazole (21 mg, 1.2 Eq, 0.15 mmol) were dissolved in DMF (1 mL). 2-Methylpropan-2-olate potassium (21 mg, 24 μL, 1.5 Eq, 0.19 mmol) was added to the reaction mixture, and it was allowed to stir at 23° C. for 5 min. The crude was then absorbed onto silica gel and purified by silica gel flash column chromatography (0-10% MeOH in DCM) to provide 7-(2-(3-(trifluoromethyl)-1H-pyrazol-1-yl)-5-(4-(trifluoromethyl)phenyl)oxazol-4-yl)-1,7-naphthyridin-8(7H)-one (10 mg, 20 μmol, 16%).

LCMS (ESI+): m/z 492.0, [M+H]⁺.

¹H NMR (400 MHz, DMSO-d6) d ppm 6.91 (d, J=7.58 Hz, 1H), 7.30 (d, J=2.69 Hz, 1H), 7.65 (d, J=7.34 Hz, 1H), 7.74 (d, J=8.31 Hz, 2H), 7.80-7.89 (m, 3H), 8.27 (d, J=8.31 Hz, 1H), 8.89 (d, J=4.40 Hz, 1H), 9.00 (d, J=2.69 Hz, 1H).

Preparation of Compound 660 Via Alternative Procedure for SNAr of Nitrogen-Containing Heterocycle with 2-halo-oxazole.

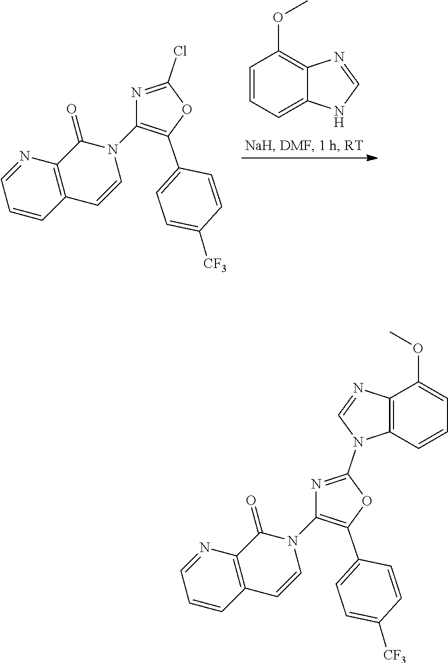 [SMILES: O=c1c2ncccc2ccn1-c1nc(Cl)oc1-c1ccc(C(F)(F)F)cc1 ; COc1cccc2[nH]cnc12 ; COc1cccc2c1ncn2-c1nc(-n2ccc3cccnc3c2=O)c(-c2ccc(C(F)(F)F)cc2)o1]

### 7-[2-(4-methoxy-1H-1,3-benzodiazol-1-yl)-5-[4-(trifluoromethyl)phenyl]-1,3-oxazol-4-yl]-7,8-dihydro-1,7-naphthyridin-8-one. Compound 660

A solution of 4-methoxy-1H-1,3-benzodiazole (0.040 g, 0.256 mmol, 1.01 eq) in anhydrous DMF (2.1 mL) was bubbled with argon for 10 minutes and cooled down to 0° C. Sodium hydride (0.015 g, 0.375 mmol, 1.47 eq) was then added. The reaction mixture was stirred for 15 min at 0° C. Next, 7-{2-chloro-5-[4-(trifluoromethyl)phenyl]-1,3-oxazol-4-yl}-7,8-dihydro-1,7-naphthyridin-8-one (0.11 g, 0.256 mmol, 1.00 eq) was added and the mixture was stirred for 1 hour at room temperature. Ethyl acetate was then added, and the reaction mixture was washed with brine. The aqueous layer was then back extracted with ethyl acetate (3 times). The combined organic layers were dried over sodium sulfate and evaporated under vacuum to obtain crude solid. The product was triturated with ethyl acetate, collected by vacuum filtration, and dried to give 7-[2-(4-methoxy-1H-1,3-benzodiazol-1-yl)-5-[4-(trifluoromethyl)phenyl]-1,3-oxazol-4-yl]-7,8-dihydro-1,7-naphthyridin-8-one (0.067 g, 0.133 mmol, 51%).

LCMS (ESI+): m/z 504.1, [M+H]⁺.

¹H NMR (300 MHz, DMSO-d₆) δ 9.10 (s, 1H), 8.90 (dd, J=4.4, 1.7 Hz, 1H), 8.29 (dd, J=8.2, 1.7 Hz, 1H), 7.92-7.75 (m, 6H), 7.71 (d, J=7.4 Hz, 1H), 7.45 (t, J=8.2 Hz, 1H), 7.01 (d, J=8.1 Hz, 1H), 6.93 (d, J=7.5 Hz, 1H), 4.01 (s, 3H).

Additional Compounds were Prepared in an Analogous Manner to Compounds 658-660, Via Displacement of Halide in 2-chloro-oxazole intermediates:

| | Exact mass | Observed | LCMS |
|---|---|---|---|
| Compound | (g/mol) | m/z(+) | Assignment |
| 661 | 420.1353 | 421.1 | [M + H]+ |
| 662 | 423.1132 | 424.1 | [M + H]+ |
| 663 | 424.1336 | 425.0 | [M + H]+ |
| 664 | 428.1397 | 429.1 | [M + H]+ |
| 665 | 438.1128 | 439.0 | [M + H]+ |
| 666 | 439.1445 | 440.2 | [M + H]+ |
| 667 | 439.1445 | 440.1 | [M + H]+ |
| 668 | 440.104 | 441.0 | [M + H]+ |
| 669 | 442.1241 | 443.1 | [M + H]+ |
| 670 | 447.1707 | 448.1 | [M + H]+ |
| 671 | 449.1288 | 450.1 | [M + H]+ |
| 672 | 449.1288 | 450.0 | [M + H]+ |
| 673 | 449.1288 | 449.9 | [M + H]+ |
| 674 | 450.1304 | 451.0 | [M + H]+ |
| 675 | 454.1441 | 455.1 | [M + H]+ |
| 676 | 454.1441 | 455.1 | [M + H]+ |
| 677 | 455.1005 | 456.0 | [M + H]+ |
| 678 | 455.1394 | 456.1 | [M + H]+ |
| 679 | 464.0284 | 464.9 | [M + H]+ |
| 680 | 465.0237 | 465.9 | [M + H]+ |
| 681 | 472.1347 | 473.1 | [M + H]+ |
| 682 | 474.1304 | 475.1 | [M + H]+ |
| 683 | 475.1256 | 476.1 | [M + H]+ |
| 684 | 475.1256 | 476.1 | [M + H]+ |
| 79 | 475.1256 | 476.1 | [M + H]+ |
| 685 | 476.1209 | 477.1 | [M + H]+ |
| 686 | 479.1394 | 480.1 | [M + H]+ |
| 687 | 488.1096 | 489.1 | [M + H]+ |
| 688 | 494.1177 | 495.0 | [M + H]+ |
| 689 | 506.1002 | 507.1 | [M + H]+ |
| 690 | 514.0252 | 515.0 | [M + H]+ |
| 691 | 516.1522 | 517.2 | [M + H]+ |
| 692 | 517.1726 | 518.2 | [M + H]+ |

### Synthesis of Compound 693.

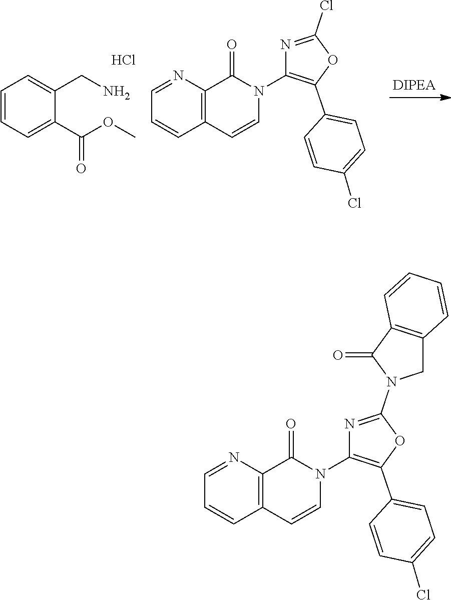

### 7-(5-(4-chlorophenyl)-2-(1-oxoisoindolin-2-yl)oxazol-4-yl)-1,7-naphthyridin-8(7H)-one. Compound 693

A solution of methyl 2-(aminomethyl)benzoate·HCl (31 mg, 1.1 Eq, 0.15 mmol) and DIPEA (45 mg, 61 μL, 2.5 Eq, 0.35 mmol) in DMF (2 mL) was stirred at room temperature for 20 minutes. At this time, 7-(2-chloro-5-(4-chlorophenyl)oxazol-4-yl)-1,7-naphthyridin-8(7H)-one (50 mg, 1 Eq, 0.14 mmol) was added and the mixture was stirred at 80° C. for 56 hours. The reaction was cooled, and the mixture was concentrated to half its original volume. Water was added and a solid precipitate formed. The solid precipitate was collected by vacuum filtration and washed with cold ethanol to obtain pure 7-(5-(4-chlorophenyl)-2-(1-oxoisoindolin-2-yl)oxazol-4-yl)-1,7-naphthyridin-8(7H)-one (13 mg, 29 μmol, 20%) as a brown solid.

LCMS (ESI+): m/z 455.0, [M+H]⁺.

¹H NMR: (400 MHz, DMSO-d6) d=8.89-8.88 (d, J=3.6 Hz, 1H), 8.28-8.21 (d, J=8.0 Hz, 1H), 7.91-7.83 (d, J=7.6 Hz, 1H), 7.81-7.74 (m, 3H), 7.64-7.61 (m, 2H), 7.57-7.55 (m, 2H), 7.45-7.43 (d, 2H), 6.88-6.86 (d, J=7.6, 1H), 5.17 (s, 2H).

### Synthesis of Compound 694.

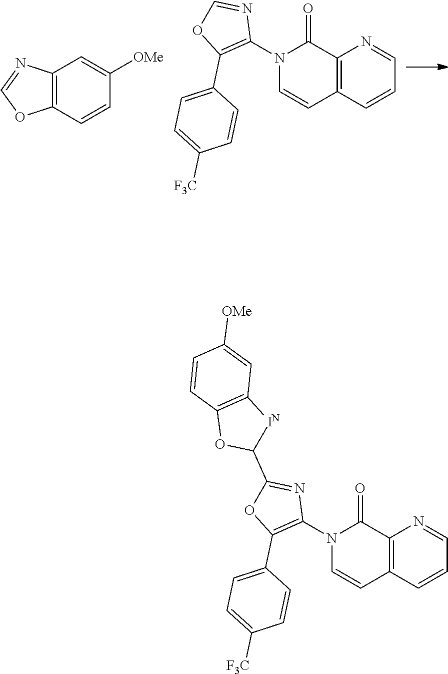

### 7-[2-(6-methoxy-1,3-benzoxazol-2-yl)-5-[4-(trifluoromethyl)phenyl]-1,3-oxazol-4-yl]-7,8-dihydro-1,7-naphthyridin-8-one. Compound 694

Under air atmosphere, a round bottom flask was charged with palladium(II) acetate (0.009 g, 0.04 mmol, 0.095 eq.), copper(II) acetate monohydrate (0.168 g, 0.841 mmol, 2.00 eq.), and Silver(I) fluoride (0.107 g, 0.843 mmol, 2.01 eq.). Then 7-(5-(4-(trifluoromethyl)phenyl)oxazol-4-yl)-1,7-naphthyridin-8(7H)-one (0.158 g, 0.42 mmol, 1.0 eq.) and 5-methoxy-1,3-benzoxazole (0.094 g, 0.63 mmol, 1.5 eq.) were added. After the addition of anhydrous DMF (6.0 mL), the mixture was stirred for 10 minutes at room temperature and then heated at 150° C. for 1 hour. After cooling to room temperature, the reaction mixture was poured into a saturated aqueous NaCl solution (40 mL) and extracted with EtOAc (3×40 mL). The organic phases were combined, and the volatile components were removed in a rotary evaporator. Purification of the crude product by silica gel column chromatography (40% EtOAc in hexanes) yielded 7-[2-(6-methoxy-1,3-benzoxazol-2-yl)-5-[4-(trifluoromethyl)phenyl]-1,3-oxazol-4-yl]-7,8-dihydro-1,7-naphthyridin-8-one (0.016 g, 0.032 mmol, 8%) as a white solid.

LCMS (ESI+): m/z 505.1, [M+H]⁺.

¹H NMR (400 MHz, DMSO-d6) d ppm 8.89 (d, J=3.67 Hz, 1H), 8.28 (d, J=7.82 Hz, 1H), 7.90 (d, J=8.31 Hz, 2H), 7.82-7.87 (m, 2H), 7.79 (d, J=8.31 Hz, 2H), 7.71 (d, J=7.34 Hz, 1H), 7.51 (d, J=2.20 Hz, 1H), 7.19 (dd, J=9.05, 2.20 Hz, 1H), 6.93 (d, J=7.34 Hz, 1H), 3.87 (s, 3H).

Additional Compounds were Synthesized in a Manner Analogous to Compound 694:

| | Exact mass | | LCMS |
|---|---|---|---|
| Compound | (g/mol) | LCMS | Assignment |
| 695 | 424.0972 | 425.1 | [M + H]+ |
| 696 | 454.1077 | 455.0 | [M + H]+ |
| 697 | 474.094 | 475.0 | [M + H]+ |
| 698 | 470.0849 | 471.0 | [M + H]+ |
| 699 | 492.0846 | 493.0 | [M + H]+ |
| 700 | 492.0846 | 493.0 | [M + H]+ |
| 701 | 492.0846 | 493.0 | [M + H]+ |
| 702 | 442.0877 | 443.0 | [M + H]+ |
| 703 | 442.0877 | 443.0 | [M + H]+ |
| 704 | 474.094 | 475.0 | [M + H]+ |
| 705 | 504.1045 | 505.1 | [M + H]+ |

### Synthesis of Compound 706.

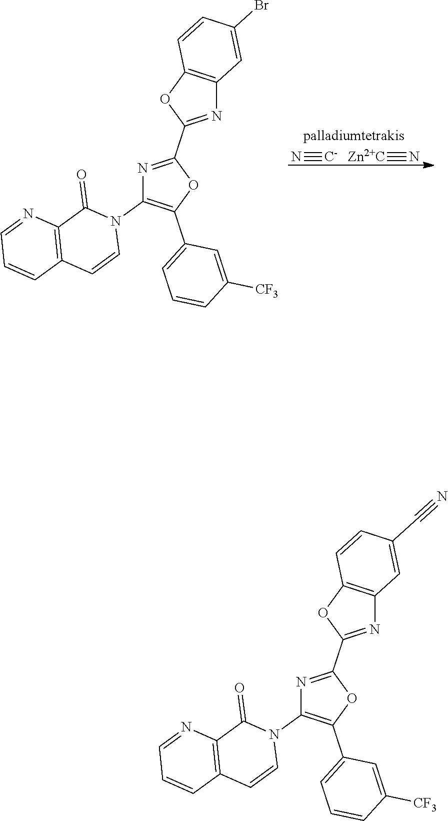

### 2-(4-(8-oxo-1,7-naphthyridin-7(8H)-3/1)-5-(3-(trifluoromethyl)phenyl)oxazol-2-yl)benzo[d]oxazole-5-carbonitrile. Compound 706

7-(2-(5-Bromobenzo[d]oxazol-2-yl)-5-(3-(trifluoromethyl)phenyl)oxazol-4-yl)-1,7-naphthyridin-8(7H)-one (300 mg, 1.0 Eq, 542 μmol), Zinc cyanide (70.0 mg, 1.1 Eq, 596 μmol), and Pd(PPh₃)₄ (188 mg, 0.3 Eq, 163 μmol) were split evenly across six vials. The vials were flushed with nitrogen before anhydrous DMF (1 mL) was added to each vial. Each of the six vials was sealed and heated in a microwave oven for 2 minutes with a set temperature of 175° C. The 6 vials were combined and poured into ethyl acetate and water. The ethyl acetate layer was separated, washed with three portions of water, and dried over magnesium sulfate. After filtration, the organic solution was concentrated to give a brown semi-solid. This material was purified by silica gel flash chromatography (0-10% MeOH in DCM) to yield 2-(4-(8-oxo-1,7-naphthyridin-7(8H)-yl)-5-(3-(trifluoromethyl)phenyl)oxazol-2-yl)benzo[d]oxazole-5-carbonitrile (10 mg, 20 μmol, 3.7%) as a beige solid.

LCMS (ESI+): m/z 500.0, [M+H]⁺.

¹H NMR: (DMSO-d6, 400 MHz) d=8.91-8.89 (dd, J=4.4 Hz, 1.6 Hz, 1H), 8.67-8.66 (d, J=1.2 Hz, 1H), 8.31-8.29 (dd, J=8.0 Hz, 1.6 Hz, 1H), 8.23-8.21 (d, J=8.8 Hz, 1H), 8.10-8.08 (dd, J=8.4 Hz, 1.2 Hz, 1H), 7.91-7.84 (m, 4H), 7.80-7.76 (t, J=8.00 Hz, 1H), 7.73-7.71 (d, J=7.6 Hz, 1H), 6.96-6.94 (d, J=7.2 Hz, 1H).

### Alternative Synthesis of Compound 694 Via Dehydrative Cyclization in the Presence of Hexachloroethane and Triarylphosphine.

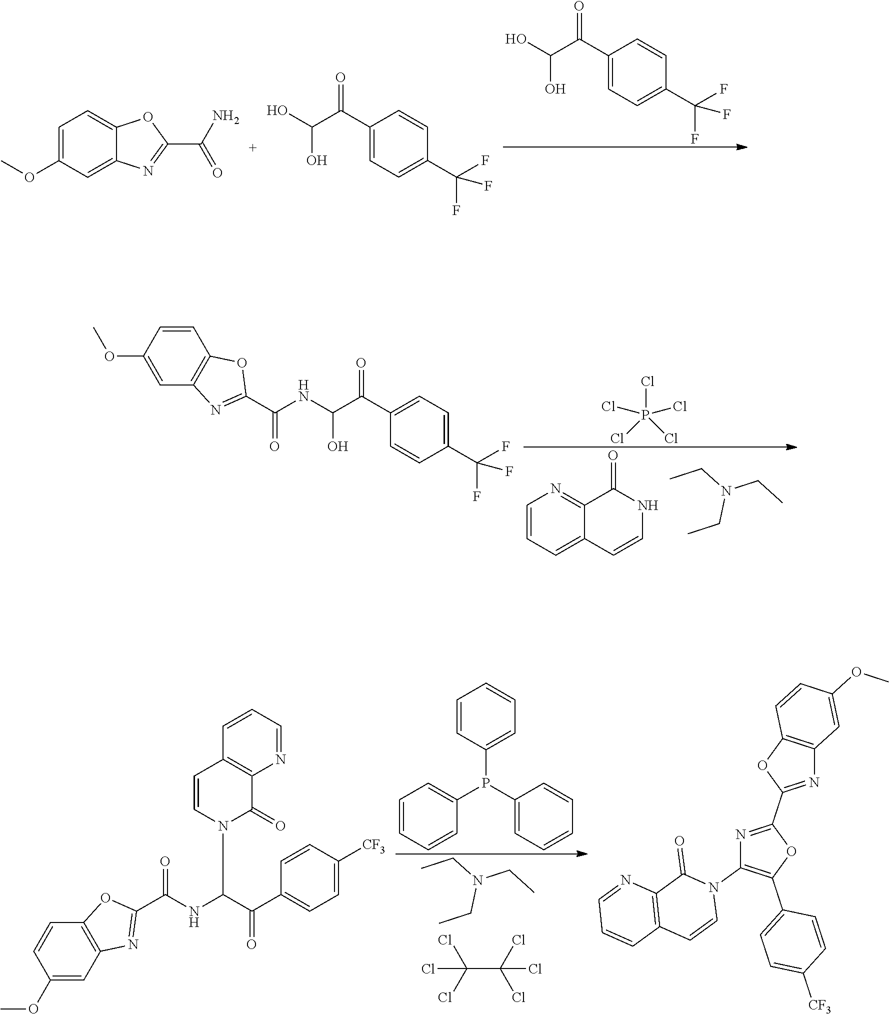

### 7-(2-(5-methoxybenzo[d]oxazol-2-yl)-5-(4-(trifluoromethyl)phenyl)oxazol-4-yl)-1,7-naphthyridin-8(7H)-one

To a slurry of 2,2-dihydroxy-1-(4-(trifluoromethyl)phenyl)ethan-1-one (2.1 g, 1.1 Eq, 9.7 mmol) in 1,4-Dioxane (30 mL) was added 5-methoxybenzo[d]oxazole-2-carboxamide (1.7 g, 1 Eq, 8.8 mmol), and the reaction mixture was heated to 90° C. for 19 hours. At this time, more 2,2-dihydroxy-1-(4-(trifluoromethyl)phenyl)ethan-1-one (0.78 g, 0.4 Eq, 3.5 mmol) and 1,4-Dioxane (20 mL) were added and stirring was continued at 90° C. for 48 hours. At this time, the reaction was cooled to room temperature, concentrated, and the resulting paste was triturated with water. The resulting precipitate was collected by vacuum filtration, washed with cold water, and dried under vacuum at 50° C. for 16 hours to give N-(1-hydroxy-2-oxo-2-(4-(trifluoromethyl)phenyl)ethyl)-5-methoxybenzo[d]oxazole-2-carboxamide (3.05 g, 7.74 mmol, 87%) as light brown solid.

### 5-methoxy-N-(2-oxo-1-(8-oxo-1,7-naphthyridin-7(8H)-yl)-2-(4-(trifluoromethyl)phenyl)ethyl)benzo[d]oxazole-2-carboxamide

To a slurry of N-(1-hydroxy-2-oxo-2-(4-(trifluoromethyl)phenyl)ethyl)-5-methoxybenzo[d]oxazole-2-carboxamide (0.630 g, 1 Eq, 1.60 mmol) in DCM (8 mL) was added phosphorous pentachloride (349 mg, 1.05 Eq, 1.68 mmol). The resulting homogeneous solution was stirred at 50° C. for 2 hours, cooled to room temperature, concentrated, and dried under vacuum to give the chloro-adduct as a yellow solid. Separately, to a solution of 1,7-naphthyridin-8(7H)-one (257 mg, 1.1 Eq, 1.76 mmol) in DMF (5 mL) was added triethylamine (485 mg, 0.66 mL, 3 Eq, 4.79 mmol), and the mixture was stirred at room temperature for 15 minutes. The reaction mixture was chilled to 0° C. and treated with the chloro-adduct and the flask containing the Chloro-adduct was rinsed with 3 mL DMF and added to the reaction mixture. The cold bath was removed, and the resulting dark brown reaction mixture was stirred at ambient temperature for 30 minutes. The mixture was then treated with water (40 mL) and the resulting precipitate was collected by vacuum filtration, washed with cold water, and dried under vacuum at 50° C. to give 5-methoxy-N-(2-oxo-1-(8-oxo-1,7-naphthyridin-7(8H)-yl)-2-(4-(trifluoromethyl)phenyl)ethyl)benzo[d]oxazole-2-carboxamide (644.0 mg, 1.23 mmol, 77.2%) as a brown solid.

### 7-(2-(5-methoxybenzo[d]oxazol-2-yl)-5-(4-(trifluoromethyl)phenyl)oxazol-4-yl)-1,7-naphthyridin-8(7H)-one. Compound 694

To a slurry of 5-methoxy-N-(2-oxo-1-(8-oxo-1,7-naphthyridin-7(8H)-yl)-2-(4-(trifluoromethyl)phenyl)ethyl)benzo[d]oxazole-2-carboxamide (1.86 g, 1 Eq, 3.56 mmol), hexachloroethane (2.11 g, 2.5 Eq, 8.90 mmol), and triphenylphosphine polymer-bound (2.97 g, 78.6% Wt, 2.5 Eq, 8.90 mmol) in acetonitrile (60 mL) was added triethylamine (1.44 g, 1.98 mL, 4.0 Eq, 14.2 mmol) and the resulting dark brown reaction mixture was heated to 60° C. for 1.5 hours. The reaction was filtered to remove the polymer, which was then washed with DCM (4×50 mL). The liquid filtrate was concentrated and triturated with water. The precipitate was collected by vacuum filtration and washed with cold water to give product as a brown solid. This material was triturated with hot MeOH, collected by vacuum filtration, washed with cold MeOH, and dried in vacuo to give 7-(2-(5-methoxybenzo[d]oxazol-2-yl)-5-(4-(trifluoromethyl)phenyl)oxazol-4-yl)-1,7-naphthyridin-8(7H)-one (1.23 g, 2.44 mmol, 68.5%).

LCMS (ESI+): m/z 505.08, [M+H]⁺.

¹H NMR (400 MHz, DMSO-d6) d ppm 8.89 (d, J=3.67 Hz, 1H), 8.28 (d, J=7.82 Hz, 1H), 7.90 (d, J=8.31 Hz, 2H), 7.82-7.87 (m, 2H), 7.79 (d, J=8.31 Hz, 2H), 7.71 (d, J=7.34 Hz, 1H), 7.51 (d, J=2.20 Hz, 1H), 7.19 (dd, J=9.05, 2.20 Hz, 1H), 6.93 (d, J=7.34 Hz, 1H), 3.87 (s, 3H).

Additional Compounds were Synthesized in an Analogous Manner to Compound 694 Via Dehydrative Cyclization in the Presence of Hexachloroethane and Triarylphosphine.

| | Exact mass | Observed | LCMS |
|---|---|---|---|
| Compound | (g/mol) | m/z(+) | Assignment |
| 707 | 423.0245 | 424.3 | [M + H]+ |
| 708 | 423.1019 | 424.1 | [M + H]+ |
| 709 | 423.1132 | 424.1 | [M + H]+ |
| 710 | 428.1285 | 428.9 | [M + H]+ |
| 711 | 431.1081 | 432.1 | [M + H]+ |
| 712 | 452.1096 | 453.0 | [M + H]+ |
| 713 | 454.1077 | 455.0 | [M + H]+ |
| 714 | 458.0461 | 458.8 | [M + H]+ |
| 715 | 459.1195 | 460.1 | [M + H]+ |
| 716 | 460.1147 | 460.9 | [M + H]+ |
| 717 | 460.1147 | 461.1 | [M + H]+ |
| 718 | 460.1535 | 461.1 | [M + H]+ |
| 719 | 463.1256 | 464.2 | [M + H]+ |
| 720 | 473.11 | 474.2 | [M + H]+ |
| 721 | 474.1304 | 475.2 | [M + H]+ |
| 722 | 474.1692 | 475.4 | [M + H]+ |
| 723 | 475.0306 | 475.9 | [M + H]+ |
| 724 | 475.0347 | 476.0 | [M + H]+ |
| 725 | 477.1413 | 478.1 | [M + H]+ |
| 726 | 478.1053 | 479.1 | [M + H]+ |
| 727 | 478.1053 | 479.1 | [M + H]+ |
| 728 | 478.1253 | 479.2 | [M + H]+ |
| 729 | 480.0989 | 481.2 | [M + H]+ |
| 730 | 482.0946 | 483.1 | [M + H]+ |
| 731 | 487.1256 | 488.1 | [M + H]+ |
| 732 | 488.1096 | 488.9 | [M + H]+ |
| 733 | 488.1209 | 489.1 | [M + H]+ |
| 734 | 488.146 | 489.2 | [M + H]+ |
| 735 | 488.1848 | 489.3 | [M + H]+ |
| 736 | 490.0889 | 491.1 | [M + H]+ |
| 737 | 492.0846 | 493.0 | [M + H]+ |
| 738 | 499.1256 | 500.1 | [M + H]+ |
| 739 | 501.1249 | 502.1 | [M + H]+ |
| 740 | 502.1253 | 503.1 | [M + H]+ |
| 741 | 504.1045 | 505.0 | [M + H]+ |
| 742 | 504.1045 | 505.0 | [M + H]+ |
| 743 | 507.0598 | 508.0 | [M + H]+ |
| 744 | 508.055 | 508.9 | [M + H]+ |
| 745 | 510.061 | 511.1 | [M + H]+ |
| 746 | 514.1253 | 515.2 | [M + H]+ |
| 747 | 516.1409 | 517.1 | [M + H]+ |
| 748 | 522.0951 | 523.0 | [M + H]+ |
| 749 | 522.0951 | 523.0 | [M + H]+ |
| 750 | 530.1202 | 530.8 | [M + H]+ |
| 751 | 534.1151 | 535.0 | [M + H]+ |
| 752 | 534.1151 | 535.0 | [M + H]+ |
| 753 | 537.0703 | 538.0 | [M + H]+ |
| 754 | 538.0656 | 539.0 | [M + H]+ |
| 755 | 538.1064 | 539.3 | [M + H]+ |

### Synthesis of Compound 756.

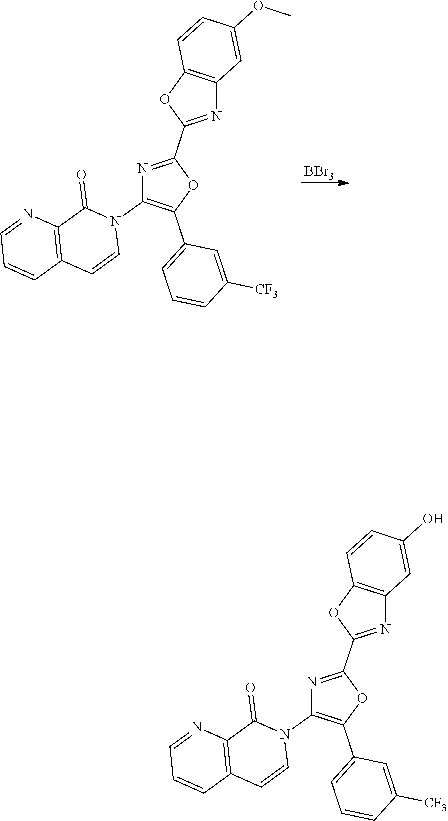 [SMILES: COc1ccc2oc(-c3nc(-n4ccc5cccnc5c4=O)c(-c4cccc(C(F)(F)F)c4)o3)nc2c1 ; "BBr3" ; O=c1c2ncccc2ccn1-c1nc(-c2nc3cc(O)ccc3o2)oc1-c1cccc(C(F)(F)F)c1]

### 7-(2-(5-hydroxybenzo[d]oxazol-2-yl)-5-(4-(trifluoromethyl)phenyl)oxazol-4-yl)-1,7-naphthyridin-8(7H)-one. Compound 756

To a solution of 7-(2-(5-methoxybenzo[d]oxazol-2-yl)-5-(4-(trifluoromethyl)phenyl)oxazol-4-yl)-1,7-naphthyridin-8(7H)-one (0.181 g, 1 Eq, 359 μmol) in DCM (5 mL) at −78° C. was added BBr₃ (117 mg, 44.1 μL, 1.3 Eq, 466 μmol), and the resulting dark brown reaction mixture was stirred at −78° C. for 6 hours. The cold bath was removed and the reaction was stirred at room temperature for 45 minutes. The reaction was cooled back to −78° C. and quenched with saturated aqueous NaHCO₃. The resulting precipitate was collected by vacuum filtration, washed with water, and dried in vacuo to give desired product as brown solid. The filtrate was extracted with 2×30 mL of DCM. The combined organic layers were washed with brine, dried over MgSO₄, filtered, and concentrated to a brown paste. The combined crude materials were purified by silica gel flash column chromatography (0-10% MeOH in DCM) to give 7-(2-(5-hydroxybenzo[d]oxazol-2-yl)-5-(4-(trifluoromethyl)phenyl)oxazol-4-yl)-1,7-naphthyridin-8(7H)-one (28.6 mg, 58.3 μmol, 16.3%) as light brown solid.

LCMS (ESI+): m/z 491.1, [M+H]⁺.

¹H NMR (400 MHz, DMSO-d6) δ ppm 9.85 (brs, 1H), 8.89 (brs, 1H), 8.29 (d, J=7.83 Hz, 1H), 7.90 (d, J=8.31 Hz, 2H), 7.84 (d, J=7.34 Hz, 1H), 7.79 (d, J=7.58 Hz, 2H), 7.72 (dd, J=13.08, 7.70 Hz, 2H), 7.24 (brs, 1H), 7.04 (d, J=8.56 Hz, 1H), 6.93 (d, J=7.58 Hz, 1H).

Synthesis of Compound 757 Via Copper-Catalyzed C—N Coupling to 2-chloro-oxazole Intermediates

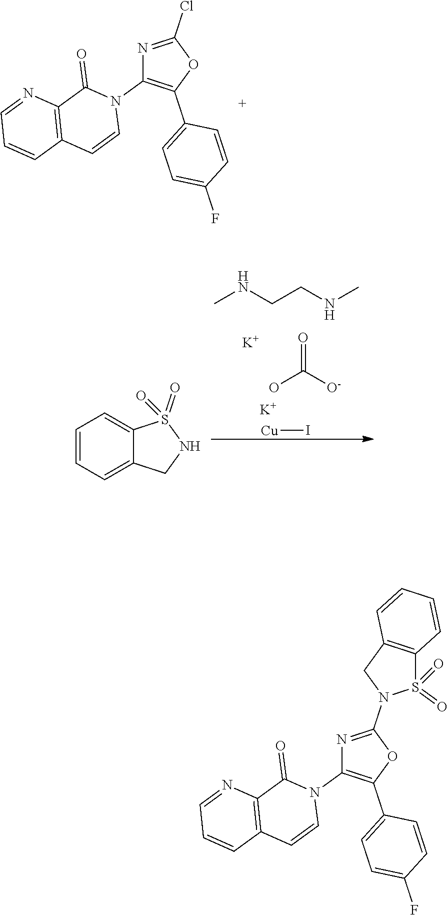

### 7-(2-(1,1-dioxidobenzo[d]isothiazol-2(3H)-yl)-5-(4-fluorophenyl)oxazol-4-yl)-1,7-naphthyridin-8(7H)-one. Compound 757

7-(2-Chloro-5-(4-fluorophenyl)oxazol-4-yl)-1,7-naphthyridin-8(7H)-one (26 mg, 1 Eq, 76 umol), 2,3-dihydrobenzo[d]isothiazole 1,1-dioxide (1.2 equiv, 15 mg, 91 umol), copper(I) iodide (2.9 mg, 0.2 Eq), potassium carbonate (2.5 Eq, 26 mg, 190 umol), N,N′-dimethylethane-1,2-diamine (0.2 Eq, 1.3 mg, 15 umol), and acetonitrile (1 mL) were added to a vial and stirred at 70° C. for 22 hours. The mixture was adsorbed onto silica and purified by silica gel column chromatography (0-5% MeOH in DCM). This purification provided 7-(2-(1,1-dioxidobenzo[d]isothiazol-2(3H)-yl)-5-(4-fluorophenyl)oxazol-4-yl)-1,7-naphthyridin-8(7H)-one (13 mg, 27 μmol, 36%) as an off-white solid.

LCMS (ESI+): m/z 475.0, [M+H]⁺.

¹H NMR: (400 MHz, DMSO-d6) δ ppm 5.39 (s, 2H) 6.86 (d, J=7.34 Hz, 1H) 7.34 (t, J=8.80 Hz, 2H) 7.45 (dd, J=8.80, 5.14 Hz, 2H) 7.65 (d, J=7.34 Hz, 1H) 7.72-7.85 (m, 3H) 7.87-7.95 (m, 1H) 8.14 (d, J=8.07 Hz, 1H) 8.24-8.29 (m, 1H) 8.88 (d, J=3.42 Hz, 1H)

Additional Compound Prepared in an Analogous Manner to Compound 757:

| | Exact mass | Observed | LCMS |
|---|---|---|---|
| Compound | (g/mol) | m/z(+) | Assignment |
| 758 | 448.1147 | 448.97 | [M + H]+ |

Example Synthesis of 2-carboxy-oxazole Intermediate.

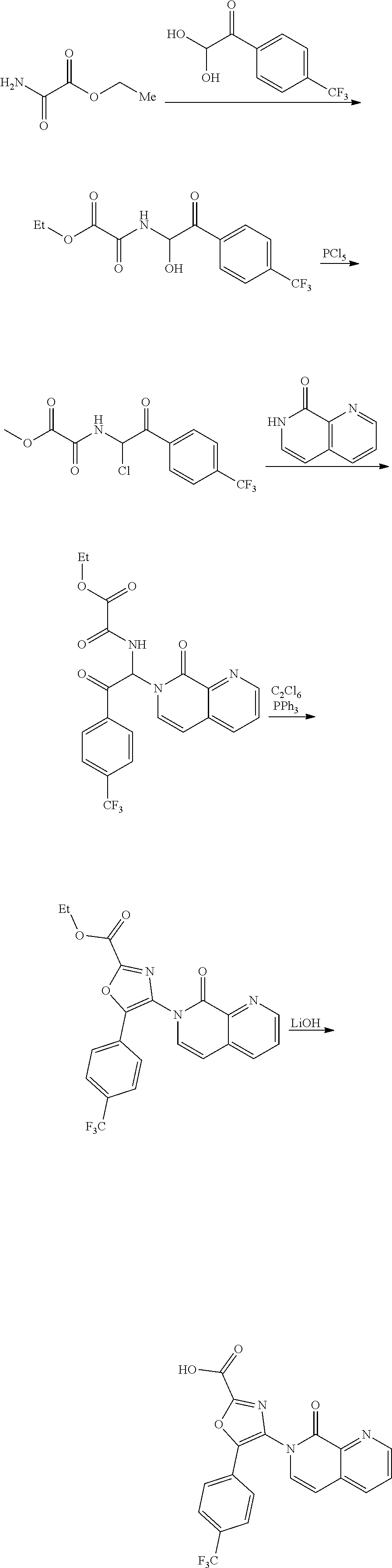

### Ethyl 2-((1-hydroxy-2-oxo-2-(4-(trifluoromethyl)phenyl)ethyl)amino)-2-oxoacetate

4-Trifluoromethylphenylglyoxal hydrate (11.0 g, 50.0 mmol, 1.0 eq) and ethyl 2-amino-2-oxoacetate (5.85 g, 50.0 mmol, 1.0 eq) were suspended in dioxane (220 mL), and the mixture was heated to 100° C. for 3 hours. Upon cooling, the dioxane was evaporated and the residue was purified by silica gel column chromatography (3-5% EtOAc in DCM) to afford the ethyl 24(1-hydroxy-2-oxo-2-(4-(trifluoromethyl)phenyl)ethyl)amino)-2-oxoacetate (8.0 g, 25.1 mmol, 50%) as a white solid.

¹H NMR (300 MHz, DMSO-d6) d 9.60 (d, J=8.0 Hz, 1H), 8.17-8.06 (m, 2H), 7.96-7.88 (m, 2H), 6.95 (d, J=7.5 Hz, 1H), 6.31 (t, J=7.8 Hz, 1H), 4.23 (q, J=7.1 Hz, 2H), 1.26 (t, J=7.1 Hz, 3H).

### ethyl 2-((1-chloro-2-oxo-2-(4-(trifluoromethyl)phenyl)ethyl)amino)-2-oxoacetate

Ethyl 2-((l-hydroxy-2-oxo-2-(4-(trifluoromethyl)phenyl)ethyl)amino)-2-oxoacetate (8.0 g, 25.1 mmol, 1.0 eq) was dissolved in dichloromethane (160 mL) and phosphorus pentachloride (5.74 g, 27.6 mmol, 1.1 eq) was added. The reaction was carried out at room temperature for 16 hours. The solvent was evaporated to afford ethyl 24(1-chloro-2-oxo-2-(4-(trifluoromethyl)phenyl)ethyl)amino)-2-oxoacetate (9.0 g, 25.1 mmol, 100%). The crude compound was used in the next step without further purification.

¹H NMR (300 MHz, DMSO-d6) d 9.60 (d, J=8.0 Hz, 1H), 8.16-8.09 (m, 2H), 7.94-7.88 (m, 2H), 6.30 (d, J=8.0 Hz, 1H), 4.23 (d, J=7.1 Hz, 2H), 1.25 (t, J=7.1 Hz, 4H).

### ethyl 2-oxo-2-((2-oxo-1-(8-oxo-1,7-naphthyridin-7(8H)-yl)-2-(4-(trifluoromethyl)phenyl)ethyl) amino)acetate

7,8-Dihydro-1,7-naphtarydin-8-one (2.73 g, 18.7 mmol, 0.7 eq) and ethyl 2-((1-chloro-2-oxo-2-(4-(trifluoromethyl)phenyl)ethyl)amino)-2-oxoacetate (9.0 g, 26.7 mmol, 1.0 eq) were dissolved in dimethylformamide (90.0 mL) and triethylamine (17.1 mL, 133 mmol, 5.0 eq) was added. The reaction was stirred at room temperature for 16 hours. The solvent was then evaporated. The crude residue was treated with water and the resulting precipitate was filtered. The precipitate was washed with a mixture of water and methanol (1/1 v/v) and then dried under vacuum. The resulting solid was suspended in diethyl ether, collected by vacuum filtration, and dried to afford ethyl 2-oxo-2-((2-oxo-1-(8-oxo-1,7-naphthyridin-7(8H)-yl)-2-(4-(trifluoromethyl)phenyl)ethyl)amino)acetate (0.66 g, 1.48 mmol, 5%).

¹H NMR (300 MHz, DMSO-d6) d 10.07 (d, J=7.9 Hz, 1H), 8.77 (dd, J=4.4, 1.6 Hz, 1H), 8.19-8.02 (m, 3H), 7.90 (d, J=8.2 Hz, 2H), 7.78-7.67 (m, 2H), 7.62 (d, J=7.9 Hz, 1H), 6.77 (d, J=7.5 Hz, 1H), 4.24 (d, J=7.1 Hz, 2H), 1.26 (t, J=7.1 Hz, 3H).

### ethyl 4-(8-oxo-1,7-naphthyridin-7(8H)-yl)-5-(4-(trifluoromethyl)phenyl)oxazole-2-carboxylate

Ethyl 2-oxo-2-((2-oxo-1-(8-oxo-1,7-naphthyridin-7(8H)-yl)-2-(4-(trifluoromethyl) phenyl) ethyl)amino) acetate (0.629 g, 1.25 mmol, 1.0 eq) was suspended in acetonitrile (11.2 mL) and hexachloroethane (0.593 g, 2.51 mmol, 2.00 eq) and triphenylphosphine (0.657 g, 2.51 mmol, 2.00 eq) were added. The solution was stirred for 10 minutes at room temperature and then pyridine (0.403 mL, 5.00 mmol, 4.0 eq) was added. The reaction was then heated to 60° C. for 2 hours. The reaction mixture was then diluted with dichloromethane and the organic layer was washed with brine 3 times. The organic layer was then dried over sodium sulfate. The crude material was purified by silica gel column chromatography (50% EtOAc in hexanes) to afford ethyl 4-(8-oxo-1,7-naphthyridin-7(8H)-yl)-5-(4-(trifluoromethyl)phenyl)oxazole-2-carboxylate (0.3 g, 0.699 mmol, 56%).

¹H NMR (300 MHz, DMSO-d6) d 8.87 (dd, J=4.4, 1.6 Hz, 1H), 8.27 (dd, J=8.1, 1.7 Hz, 1H), 7.88 (d, J=8.3 Hz, 2H), 7.73 (d, J=8.2 Hz, 2H), 7.64-7.61 (m, 2H), 6.89 (d, J=7.5 Hz, 1H), 4.46 (d, J=7.1 Hz, 2H), 1.37 (t, J=7.1 Hz, 3H).

### 4-(8-oxo-1,7-naphthyridin-7(8H)-yl)-5-(4-(trifluoromethyl)phenyl)oxazole-2-carboxylic acid

Ethyl 4-(8-oxo-7,8-dihydro-1,7-naphthyridin-7-yl)-5-[4-(trifluoromethyl)phenyl]-1,3-oxazole-2-carboxylate (0.3 g, 0.699 mmol, 1.0 eq) was suspended in a mixture of tetrahydrofuran/water (1:1, 2.7 mL) and lithium hydroxide monohydrate (0.059 g, 1.41 mmol, 2.01 eq) was added. The reaction mixture was stirred at room temperature for 15 minutes. The reaction mixture was acidified with a 1 N aqueous solution of hydrochloric acid. The resulting white precipitate was collected by vacuum filtration and dried under vacuum to afford 4-(8-oxo-7,8-dihydro-1,7-naphthyridin-7-yl)-5-[4-(trifluoromethyl)phenyl]-1,3-oxazole-2-carboxylic acid (0.17 g, 0.424 mmol, 59%) as a white solid.

LCMS (ESI+): m/z 401.95, [M+H]⁺, RT=2.42 min.

Synthesis of Compound 759 from 2-carboxy-oxazole Intermediate.

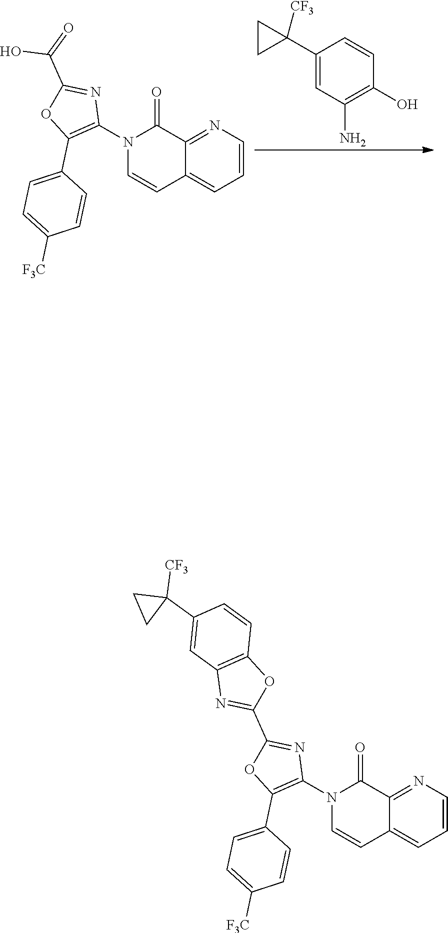 [SMILES: O=C(O)c1nc(-n2ccc3cccnc3c2=O)c(-c2ccc(C(F)(F)F)cc2)o1 ; Nc1cc(C2(C(F)(F)F)CC2)ccc1O ; O=c1c2ncccc2ccn1-c1nc(-c2nc3cc(C4(C(F)(F)F)CC4)ccc3o2)oc1-c1ccc(C(F)(F)F)cc1]

### 7-(2-{5-[1-(trifluoromethyl)cyclopropyl]-1,3-benzoxazol-2-yl}-5-[4-(trifluoromethyl)phenyl]-1,3-oxazol-4-yl)-7,8-dihydro-1,7-naphthyridin-8-one. Compound 759

A microwave reaction tube was charged with 2-amino-4-[1-(trifluoromethyl)cyclopropyl]phenol (0.033 g, 0.099 mmol, 0.991 eq), 4-(8-oxo-7,8-dihydro-1,7-naphthyridin-7-yl)-5-[4-(trifluoromethyl)phenyl]-1,3-oxazole-2-carboxylic acid (0.043 g, 0.101 mmol, 1.01 eq), triphenylphosphine (0.078 g, 0.297 mmol, 2.98 eq), trichloroacetonitrile (0.02 mL, 0.195 mmol, 1.96 eq), and anhydrous MeCN (0.8 mL). The reaction was then heated in a microwave to 170° C. for 25 minutes. Conversion to the uncyclized amide was observed by LCMS and the reaction was concentrated and the amide intermediate was purified by silica gel column chromatography (0-10% MeOH in DCM). The resulting amide intermediate was then dissolved in anhydrous acetonitrile (1.2 mL) and added to a reaction vial containing triphenylphosphine (0.052 g, 0.198 mmol, 1.99 eq) and hexachloroethane (0.047 g, 0.199 mmol, 1.99 eq). Then, pyridine (0.032 mL, 0.397 mmol, 3.99 eq) was added and the reaction was heated to reflux for 16 hours. The volatiles were evaporated, and the compound was purified by preparative-HPLC (C18 column, 10-100% MeCN in water with 0.1% TFA). Fractions containing product were combined and lyophilized to afford 7-(2-{5-[1-(trifluoromethyl)cyclopropyl]-1,3-benzoxazol-2-yl}-5-[4-(trifluoromethyl)phenyl]-1,3-oxazol-4-yl)-7,8-dihydro-1,7-naphthyridin-8-one (0.004 g, 0.007 mmol, 7%) as a beige solid.

LCMS (ESI+): m/z 583.06, [M+H]⁺.

¹H NMR (300 MHz, DMSO-d6): d 8.89 (dd, J=4.3, 1.4 Hz, 1H), 8.29 (dd, J=8.1, 1.5 Hz, 1H), 8.09 (d, J=1.2 Hz, 1H), 7.98 (d, J=8.6 Hz, 1H), 7.91 (d, J=8.4 Hz, 2H), 7.87-7.79 (m, 3H), 7.72 (d, J=7.5 Hz, 2H), 6.93 (d, J=7.5 Hz, 1H), 1.48-1.40 (m, 2H), 1.27 (s, 2H).

Additional Compounds Prepared in a Manner Analogous to Compound 759:

| | Exact mass | Observed | LCMS |
|---|---|---|---|
| Compound | (g/mol) | m/z(+) | Assignment |
| 760 | 489.1049 | 489.82 | [M + H]+ |
| 761 | 515.1205 | 515.81 | [M + H]+ |
| 762 | 556.097 | 557.11 | [M + H]+ |

### Synthesis of Compound 763.

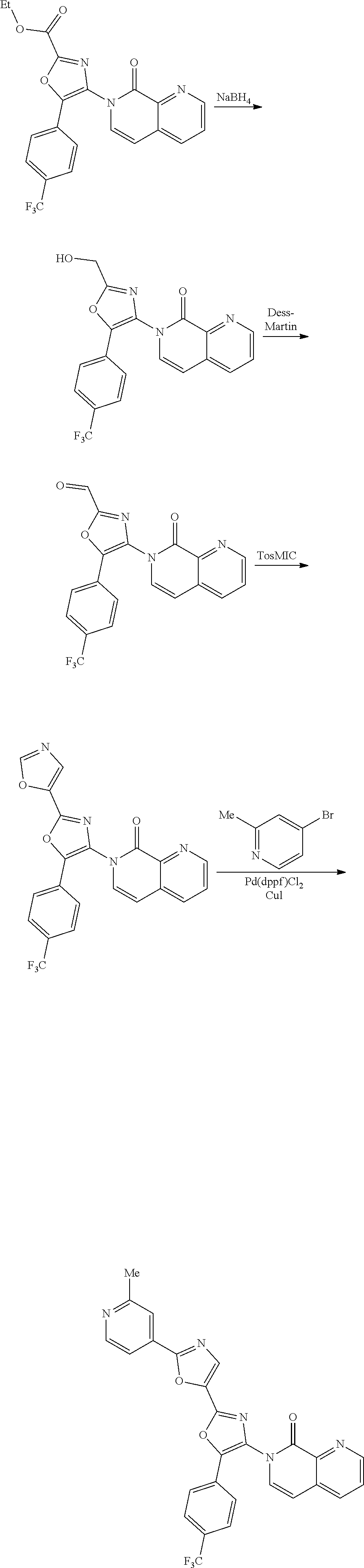

### 7-[2-(hydroxymethyl)-5-[4-(trifluoromethyl)phenyl]-1,3-oxazol-4-yl]-7,8-dihydro-1,7-naphthyridin-8-one

Ethyl 4-(8-oxo-7,8-dihydro-1,7-naphthyridin-7-yl)-5-[4-(trifluoromethyl)phenyl]-1,3-oxazole-2-carboxylate (0.417 g, 0.582 mmol, 1.00 eq) was suspended in methanol (5 mL). After cooling to 0° C., sodium borohydride (0.055 g, 1.46 mmol, 2.50 eq) was added in one portion. The reaction mixture was stirred at 0° C. for 10 minutes and then warmed to room temperature for 1 hour. The reaction mixture was quenched with water and extraction with dichloromethane was performed (3 times). The combined organic layers were washed with brine, dried over sodium sulfate, and concentrated. The crude material was purified by silica gel column chromatography (0-5% MeOH in DCM) to afford 7-[2-(hydroxymethyl)-5-[4-(trifluoromethyl)phenyl]-1,3-oxazol-4-yl]-7,8-dihydro-1,7-naphthyridin-8-one (0.228 g, 100%) as a white solid.

¹H NMR (300 MHz, DMSO-d6): d 8.87 (dd, J=4.4, 1.7 Hz, 1H), 8.26 (dd, J=8.1, 1.7 Hz, 1H), 7.92-7.72 (m, 3H), 7.62 (dd, J=7.8, 4.4 Hz, 3H), 6.85 (d, J=7.4 Hz, 1H), 5.99 (t, J=6.3 Hz, 1H), 4.68 (d, J=6.3 Hz, 2H).

### 4-(8-oxo-7,8-dihydro-1,7-naphthyridin-7-yl)-5-[4-(trifluoromethyl)phenyl]-1,3-oxazole-2-carbaldehyde

7-[2-(hydroxymethyl)-5-[4-(trifluoromethyl)phenyl]-1,3-oxazol-4-yl]-7,8-dihydro-1,7-naphthyridin-8-one (0.207 g, 0.47 mmol, 1.00 eq) was dissolved in anhydrous dichloromethane (3.64 mL) and Dess-Martin periodinane (0.239 g, 0.563 mmol, 1.20 eq) was added in one portion. The reaction mixture was stirred at room temperature for 2 hours. An aqueous sodium thiosulfate solution and aqueous sodium bicarbonate saturated solution were added followed by extraction with dichloromethane (3 times). The combined organic layers were dried over sodium sulfate and concentrated. The crude material was purified by silica gel column chromatography (0-4% MeOH in DCM) to afford 4-(8-oxo-7,8-dihydro-1,7-naphthyridin-7-yl)-5-[4-(trifluoromethyl)phenyl]-1,3-oxazole-2-carbaldehyde (0.23 g, 0.597 mmol, 100%) as a white solid.

¹H NMR (300 MHz, DMSO-d6) d 8.87 (dd, J=4.4, 1.7 Hz, 1H), 8.26 (dd, J=8.1, 1.7 Hz, 1H), 7.89-7.74 (m, 3H), 7.63 (dd, J=7.6, 5.2 Hz, 4H), 6.85 (d, J=7.4 Hz, 1H), 5.72 (d, J=7.9 Hz, 1H).

### 7-[2-(1,3-oxazol-5-yl)-5-[4-(trifluoromethyl)phenyl]-1,3-oxazol-4-yl]-7,8-dihydro-1,7-naphthyridin-8-one

4-(8-Oxo-7,8-dihydro-1,7-naphthyridin-7-yl)-5-[4-(trifluoromethyl)phenyl]-1,3-oxazole-2-carbaldehyde (0.230 g, 0.597 mmol, 1.0 eq) and potassium carbonate (0.165 g, 1.19 mmol, 2.00 eq) were suspended in methanol (4.6 mL). Next, p-toluenesulfonylmethyl isocyanide (0.128 g, 0.656 mmol, 1.1 eq) was added and the reaction mixture was stirred at reflux for 1 hour. After cooling, dichloromethane and water were added. Extraction with dichloromethane was performed (3 times). The combined organic layers were washed with brine, dried with sodium sulfate, and concentrated to afford 7-[2-(1,3-oxazol-5-yl)-5-[4-(trifluoromethyl)phenyl]-1,3-oxazol-4-yl]-7,8-dihydro-1,7-naphthyridin-8-one (0.196 g, 0.462 mmol, 77%) as an orange solid which was used in the next step without further purification.

¹H NMR (300 MHz, DMSO-d6) d 8.88 (dd, J=4.4, 1.6 Hz, 1H), 8.80 (s, 1H), 8.27 (dd, J=8.2, 1.7 Hz, 1H), 8.21 (s, 1H), 7.92-7.78 (m, 3H), 7.78-7.64 (m, 3H), 6.89 (d, J=7.4 Hz, 1H).

### 7-{2-[2-(2-methylpyridin-4-yl)-1,3-oxazol-5-yl]-5-[4-(trifluoromethyl)phenyl]-1,3-oxazol-4-yl}-7,8-dihydro-1,7-naphthyridin-8-one. Compound 763

In a reaction tube, cesium carbonate (0.23 g, 0.706 mmol, 3.00 eq) and 7-[2-(1,3-oxazol-5-yl)-5-[4-(trifluoromethyl)phenyl]-1,3-oxazol-4-yl]-7,8-dihydro-1,7-naphthyridin-8-one (0.1 g, 0.236 mmol, 1.0 eq) were dissolved in anhydrous dimethylformamide (2.0 mL). 4-Bromo-2-methylpyridine (0.056 mL, 0.472 mmol, 2.00 eq) was added and the reaction mixture was purged with argon for 5 minutes. Copper(I) iodide (0.004 g, 0.021 mmol, 0.089 eq) and 1,1′-bis(diphenylphosphino)ferrocene palladium (II) dichloride (0.017 g, 0.023 mmol, 0.099 eq) were added. The tube was sealed, and the reaction mixture was stirred at 110° C. for 16 hours. After cooling, the reaction mixture was filtered over a pad of celite, and this pad was washed with a mixture of dichloromethane/methanol (9/1 v/v). The filtrate was concentrated to give crude material which was then purified by silica gel column chromatography (0-3% MeOH in DCM) to afford 7-{2-[2-(2-methylpyridin-4-yl)-1,3-oxazol-5-yl]-5-[4-(trifluoromethyl)phenyl]-1,3-oxazol-4-yl}-7,8-dihydro-1,7-naphthyridin-8-one (0.093 g, 0.18 mmol, 72%).

LCMS (ESI+): m/z 516.17, [M+H]⁺.

¹H NMR (400 MHz, DMSO-d6) d 8.90 (s, 1H), 8.71 (d, J=5.1 Hz, 1H), 8.44 (s, 1H), 8.29 (dd, J=8.1, 1.5 Hz, 1H), 7.95-7.82 (m, 5H), 7.79 (d, J=8.3 Hz, 2H), 7.71 (d, J=7.4 Hz, 1H), 6.92 (d, J=7.4 Hz, 1H), 2.62 (s, 3H).

| | Exact mass | Observed | LCMS |
|---|---|---|---|
| Compound | (g/mol) | m/z(+) | Assignment |
| 763 | 515.1205 | 516.17 | [M + H]+ |

### Synthesis of Aryl and Heteroaryl Primary Amides

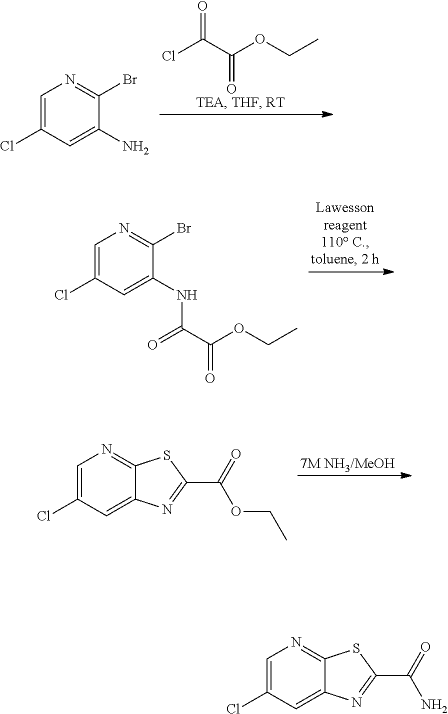

### Ethyl [(2-bromo-5-chloropyridin-3-yl)carbamoyl]formate

Ethyl chlorooxoacetate (1.62 mL, 14.5 mmol, 1.5 eq) was added dropwise to a cooled (0° C.) mixture of 3-amino-2-bromo-5-chloropyridine (2.0 g, 9.64 mmol, 1.0 eq) and triethylamine (2.69 mL, 19.3 mmol, 2.0 eq) in tetrahydrofuran (30.0 mL). The reaction mixture was allowed to warm slowly to room temperature and stirred for 12 hours. The mixture was then filtered to remove the ammonium salts, and the filtrate was concentrated under vacuum yielding ethyl [(2-bromo-5-chloropyridin-3-yl)carbamoyl]formate (2.97 g, 9.64 mmol, 67%). This crude material was used in the next step without further purification.

LCMS (ESI+): m/z 308.80, [M+H]⁺.

### Ethyl 6-chloro-[1,3]thiazolo[5,4-b]pyridine-2-carboxylate

Lawesson's Reagent (1.58 g, 3.90 mmol, 0.5 eq) was added to a mixture of ethyl [(2-bromo-5-chloropyridin-3-yl)carbamoyl]formate (3.58 g, 7.80 mmol, 1.0 eq) in anhydrous toluene (48.0 mL). The reaction mixture was stirred at 110° C. for 4 hours. The mixture was then concentrated and purified by silica gel column chromatography (100% DCM) to give ethyl 6-chloro-[1,3]thiazolo[5,4-b]pyridine-2-carboxylate (1.5 g, 6.18 mmol, 76%) as white solid.

LCMS (ESI+): m/z 242.85, [M+H]⁺.

### 6-chloro-[1,3]thiazolo[5,4-b]pyridine-2-carboxamide

Ethyl 6-chloro-[1,3]thiazolo[5,4-b]pyridine-2-carboxylate (1.50 g, 6.18 mmol, 1.0 eq) was suspended in a 7 M ammonia solution in methanol (8.83 mL, 10.0 eq), and the mixture was stirred at room temperature for 3 hours. The precipitate from the reaction was filtered, washed with diethyl ether, and dried to afford pure 6-chloro-[1,3]thiazolo[5,4-b]pyridine-2-carboxamide (1.10 g, 5.13 mmol, 83%) as a white solid.

¹H NMR (300 MHz, DMSO-d6) δ 8.84 (d, J=2.2 Hz, 1H), 8.72 (d, J=2.2 Hz, 1H), 8.61 (s, 1H), 8.26 (s, 1H).

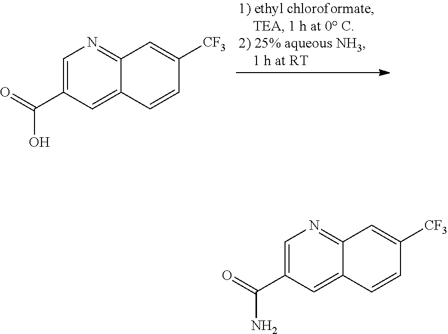 [SMILES: O=C(O)c1cnc2cc(C(F)(F)F)ccc2c1 ; NC(=O)c1cnc2cc(C(F)(F)F)ccc2c1]

### 7-(trifluoromethyl)quinoline-3-carboxamide

7-(Trifluoromethyl)quinoline-3-carboxylic acid (1.0 g, 4.15 mmol, 1.0 eq) was dissolved in tetrahydrofuran (15.0 mL). Triethylamine (0.694 mL, 4.98 mmol, 1.20 eq) was added and the resulting mixture was cooled to 0° C. Ethyl chloroformate (0.325 mL, 4.98 mmol, 1.2 eq) was added dropwise, and the reaction was stirred at 0° C. for 1 hour. Then, 25% aqueous ammonia (3.54 mL, 20.7 mmol, 5.0 eq) was added, and the mixture was allowed to warm to room temperature and stirred for 1 hour. Ethyl acetate was added and the organic layer was washed with saturated aqueous sodium bicarbonate solution, dried over sodium sulfate and concentrated under reduced pressure to give 7-(trifluoromethyl)quinoline-3-carboxamide (0.961 g, 4.00 mmol, 68%).

1H NMR (300 MHz, Chloroform-d) d 9.42 (d, J=2.2 Hz, 1H), 8.75 (s, 1H), 8.51 (s, 1H),

8.10 (d, J=8.5 Hz, 1H), 7.84 (d, J=9.1 Hz, 1H).

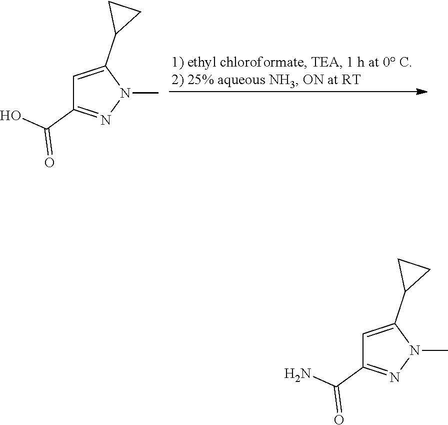

### 5-cyclopropyl-1-methyl-1H-pyrazole-3-carboxamide

5-Cyclopropyl-1-methyl-1H-pyrazole-3-carboxylic acid (0.5 g, 3.00 mmol, 1.0 eq) was dissolved in anhydrous tetrahydrofuran (7.5 mL). Triethylamine (0.503 mL, 3.61 mmol, 1.20 eq) was then added. The mixture was cooled to 0° C. and ethyl chloroformate (0.236 mL, 3.61 mmol, 1.20 eq) was added slowly. Stirring was continued at 0° C. for 1 hour. 25% Ammonia in water (10.3 mL, 15.0 mmol, 5.0 eq) was added, and the mixture was stirred at room temperature for 16 hours. Dichloromethane and a saturated aqueous solution of sodium bicarbonate were added to the reaction mixture and the layers were separated. The water layer was extracted 3 times with dichloromethane. The organic layers were combined, dried over anhydrous sodium sulfate, and concentrated. The crude material was triturated with dichloromethane in hexanes (1:9). The resulting precipitate was collected by vacuum filtration and dried to give pure 5-cyclopropyl-1-methyl-1H-pyrazole-3-carboxamide (0.35 g, 2.20 mmol, 67%).

¹H NMR (300 MHz, DMSO-d₆) δ 7.31 (s, 1H), 7.08 (s, 1H), 6.24 (d, J=0.7 Hz, 1H), 3.86 (s, 3H), 1.96-1.82 (m, 1H), 1.02-0.88 (m, 2H), 0.71-0.59 (m, 2H).

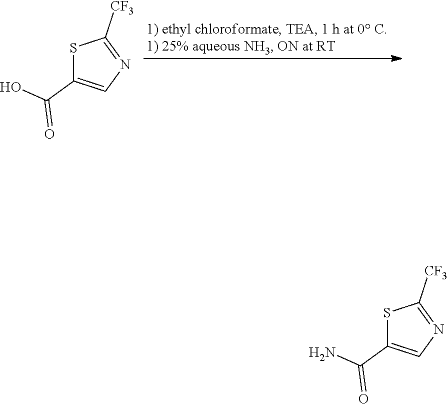 [SMILES: O=C(O)c1cnc(C(F)(F)F)s1 ; NC(=O)c1cnc(C(F)(F)F)s1]

### 2-(trifluoromethyl)-1,3-thiazole-5-carboxamide

2-(Trifluoromethyl)-1,3-thiazole-5-carboxylic acid (1.0 g, 5.07 mmol, 1.0 eq) was dissolved in tetrahydrofuran (15.0 mL). Triethylamine (0.848 mL, 6.08 mmol, 1.20 eq) was added. The mixture was cooled to 0° C. and ethyl chloroformate (0.397 mL, 6.08 mmol, 1.20 eq) was added slowly. Stirring was continued at 0° C. for 1 hour. Then, 25% aqueous ammonia (4.34 mL, 25.4 mmol, 5.0 eq) was added, and the mixture was stirred at room temperature for 16 hours. Dichloromethane and a saturated aqueous solution of sodium bicarbonate were added to the reaction mixture and the layers were separated. The water layer was extracted 3 times with dichloromethane. The organic layers were combined, dried over anhydrous sodium sulfate, and concentrated to give 2-(trifluoromethyl)-1,3-thiazole-5-carboxamide (0.71 g, 3.62 mmol, 68%).

¹H NMR (300 MHz, DMSO-d₆) δ 8.62 (d, J=1.3 Hz, 1H), 8.48 (s, 1H), 8.21-7.85 (m, 1H).

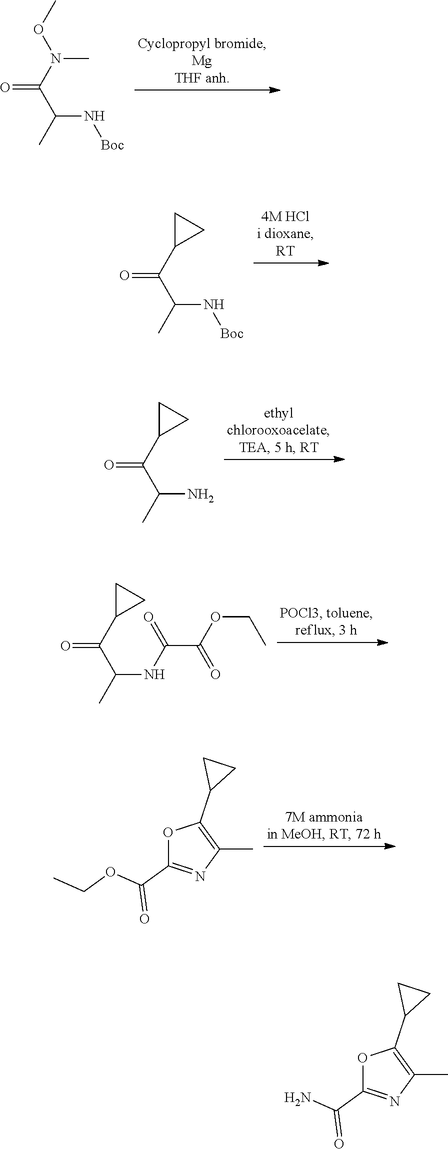

### tert-butyl N-(1-cyclopropyl-1-oxopropan-2-yl)carbamate

Into a flame dried reaction flask were added magnesium turnings (6.59 g, 271 mmol, 4.2 eq) and several crystals of iodine under argon. Anhydrous diethyl ether (50.0 mL) was added to the flask, followed by dropwise addition of a solution of cyclopropyl bromide (31.2 g, 258 mmol, 2.2 eq) in diethyl ether (50.0 mL). The reaction was heated to reflux and stirred until the full disappearance of magnesium turnings (1 hour). Then, the reaction mixture was cooled to 0° C. To the magnesium cyclopropyl bromide solution was added dropwise a solution of (S)-tert-Butyl 1-(methoxy(methyl)amino)-1-oxopropan-2-ylcarbamate (15.0 g, 64.6 mmol, 1.0 eq) in anhydrous tetrahydrofuran (300 mL). The mixture was allowed to warm to room temperature and stirred for 16 hours. The reaction was cooled to 0° C. and quenched by the addition of acetic acid (50.0 mL). The mixture was diluted with water and dichloromethane, and the layers were separated. The water layer was extracted twice more with dichloromethane. The organic layers were combined, washed twice with a saturated aqueous solution of sodium bicarbonate, dried over sodium sulfate, and concentrated to give crude material. The crude material was purified by silica gel column chromatography (0-20% EtOAc in hexanes). The fractions containing desired product were collected and concentrated to give tert-butyl N-(1-cyclopropyl-1-oxopropan-2-yl)carbamate (13.0 g, 61.0 mmol, 85%).

¹H NMR (300 MHz, DMSO-d₆) δ 7.23 (d, J=7.4 Hz, 1H), 4.13 (t, J=7.3 Hz, 1H), 2.16 (ddd, J=7.7, 5.0, 3.2 Hz, 1H), 1.39 (s, 11H), 1.18 (s, 2H), 0.93-0.67 (m, 4H).

### 2-amino-1-cyclopropylpropan-1-one

Tert-butyl N-(1-cyclopropyl-1-oxopropan-2-yl)carbamate (14.4 g, 61.0 mmol, 1.0 eq) was dissolved in dioxane (130 mL). 4 N HCl in dioxane, (76.2 mL, 305 mmol, 5.0 eq) was slowly added. The reaction was carried out at room temperature for 16 hours. The mixture was concentrated to dryness, suspended in diethyl ether, and concentrated to dryness again. The solid was then suspended again in diethyl ether, and the resulting suspension was stirred for 15 minutes. The precipitate was collected by vacuum filtration, washed with diethyl ether, and dried to give 2-amino-1-cyclopropylpropan-1-one. The crude was used in the next step without further purification or characterization.

### Ethyl [(1-cyclopropyl-1-oxopropan-2-yl)carbamoyl]formate

Ethyl chlorooxoacetate (1.19 mL, 10.6 mmol, 1.2 eq) was added dropwise to a cooled (0° C.) mixture of 2-amino-1-cyclopropylpropan-1-one (1.05 g, 8.84 mmol, 1.0 eq) and triethylamine (1.48 mL, 10.6 mmol, 1.2 eq) dissolved in anhydrous dichloromethane (20.0 mL). The reaction mixture was stirred for 5 hours at room temperature. Water was added and the layers were separated. The organic layer was washed three times with water, dried over anhydrous sodium sulfate, and evaporated. The crude material was purified by silica gel column chromatography (0-5% methanol in dichloromethane) to give ethyl [(1-cyclopropyl-1-oxopropan-2-yl)carbamoyl]formate (1.34 g, 6.28 mmol, 67%).

¹H NMR (300 MHz, DMSO-d₆) δ 9.12 (d, J=7.4 Hz, 1H), 4.54 (p, J=7.3 Hz, 1H), 4.24 (q, J=7.1 Hz, 2H), 2.17 (tt, J=7.8, 4.6 Hz, 1H), 1.38-1.22 (m, 6H), 0.99-0.72 (m, 4H).

### ethyl 5-cyclopropyl-4-methyl-1,3-oxazole-2-carboxylate

Ethyl [(1-cyclopropyl-1-oxopropan-2-yl)carbamoyl]formate (8.84 g, 39.4 mmol, 1.0 eq) was dissolved in anhydrous toluene (168 mL). Then, phosphorus(V) oxychloride (7.34 mL, 78.8 mmol, 2.0 eq) was slowly added. The reaction was carried out at reflux for 3 hours. After cooling, the solvent was evaporated and the crude material was purified by silica gel column chromatography (10-50% EtOAc in hexanes) to give ethyl 5-cyclopropyl-4-methyl-1,3-oxazole-2-carboxylate (2.43 g, 12.4 mmol, 30%).

¹H NMR (300 MHz, DMSO-d₆) δ 4.30 (q, J=7.1 Hz, 2H), 2.16 (s, 3H), 2.07 (tt, J=8.4, 5.1 Hz, 1H), 1.28 (t, J=7.1 Hz, 3H), 1.06-0.95 (m, 2H), 0.88-0.77 (m, 2H).

### 5-cyclopropyl-4-methyl-1,3-oxazole-2-carboxamide

Ethyl 5-cyclopropyl-4-methyl-1,3-oxazole-2-carboxylate (1.43 g, 6.91 mmol, 1.0 eq) was suspended in 7 M ammonia in methanol (5.49 ml, 38.4 mmol, 5.0 eq), and the mixture was stirred at room temperature for 48 hours. The solvent was evaporated, the resulting solid suspended in diethyl ether, and the resulting precipitate collected by vacuum filtration. This material was washed with diethyl ether and dried to give 5-cyclopropyl-4-methyl-1,3-oxazole-2-carboxamide (0.996 g, 5.99 mmol, 76%).

¹H NMR (300 MHz, DMSO-d₆) δ 8.01 (s, 1H), 7.67 (s, 1H), 2.14 (s, 4H), 1.03-0.93 (m, 2H), 0.86-0.78 (m, 2H).

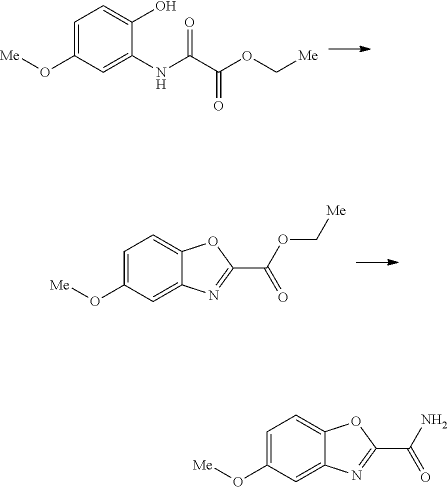 [SMILES: CCOC(=O)C(=O)Nc1cc(OC)ccc1O ; CCOC(=O)c1nc2cc(OC)ccc2o1 ; COc1ccc2oc(C(N)=O)nc2c1]

### ethyl 2-((2-hydroxy-5-methoxyphenyl)amino)-2-oxoacetate

To a 0° C. solution of 2-amino-4-methoxyphenol (20.5 g, 1 Eq, 148 mmol) in THF (400 mL) was added a solution of ethyl chloroglyoxalate (24.2 g, 19.8 mL, 1.2 Eq, 177 mmol) in THF (50 mL) over 15 minutes. The dropping funnel was rinsed with another portion of THF (50 mL). The cold bath was removed, and the reaction was stirred at ambient temperature for 1 hour. The resulting precipitate was collected by vacuum filtration, washed with Et₂O, and dried under vacuum at 50° C. to give crude desired product as light purple solid. The liquid filtrate was concentrated, and the resulting solid was washed with Et₂O and dried to give more product. The combined material gave ethyl 2-((2-hydroxy-5-methoxyphenyl)amino)-2-oxoacetate (28.8 g, 120 mmol, 81.6%) as light purple solid.

¹H NMR (400 MHz, DMSO-d₆) d ppm 9.80 (s, 1H), 9.58 (s, 1H), 7.65 (d, J=2.69 Hz, 1H), 6.83 (d, J=8.80 Hz, 1H), 6.61 (dd, J=8.80, 2.69 Hz, 1H), 4.29 (q, J=7.09 Hz, 2H), 3.38 (brs, 1H), 1.30 (t, J=7.09 Hz, 3H).

LCMS (ESI+): m/z 240.0, [M+H]⁺.

### Ethyl 5-methoxybenzo[d]oxazole-2-carboxylate

To a solution of ethyl 2-((2-hydroxy-5-methoxyphenyl)amino)-2-oxoacetate (5.50 g, 1 Eq, 23.0 mmol), hexachloroethane (7.62 g, 1.4 Eq, 32.2 mmol), and triphenylphosphine (8.44 g, 1.4 Eq, 32.2 mmol) in acetonitrile (150 mL) was added triethylamine (6.98 g, 9.61 mL, 3.0 Eq, 69.0 mmol), and the resulting dark brown reaction mixture was heated to 60° C. After 30 minutes, the reaction was complete, and the reaction was let cool and filtered to remove Et₃N·HCl. The liquid filtrate was diluted with 200 mL of EtOAc and washed with H₂O (150 mL). The organic layer was washed with brine, dried over MgSO₄, filtered, and concentrated to give a dark brown paste. The crude material was purified by silica gel flash column chromatography (0-100% EtOAc in heptane) to give ethyl 5-methoxybenzo[d]oxazole-2-carboxylate (3.49 g, 15.8 mmol, 68.6%) as an orange solid.

LCMS (ESI+): m/z 222.0, [M+H]⁺.

### 5-methoxybenzo[d]oxazole-2-carboxamide

To a slurry of ethyl 5-methoxybenzo[d]oxazole-2-carboxylate (2.0 g, 1 Eq, 9.0 mmol) in MeOH (15 mL) in a 50 mL pressure flask was added a 7.0 M solution of ammonia in MeOH (1.5 g, 13 mL, 7 molar, 10 Eq, 90 mmol). The reaction was stirred at room temperature for 15 minutes. The reaction was concentrated and dried under vacuum at 50° C. to give 5-methoxybenzo[d]oxazole-2-carboxamide (1.73 g, 9.00 mmol, 100%) as pink solid.

¹H NMR (400 MHz, CHLOROFORM-d) d ppm 8.07 (d, J=7.58 Hz, 1H), 7.91 (d, J=8.31 Hz, 1H), 7.47-7.59 (m, 1H), 7.37-7.44 (m, 1H), 7.35 (d, J=5.38 Hz, 1H), 7.29-7.33 (m, 1H), 7.13 (d, J=5.38 Hz, 1H).

LCMS (ESI+): m/z 193.0, [M+H]⁺.

### Glyoxal Hydrate Synthesis

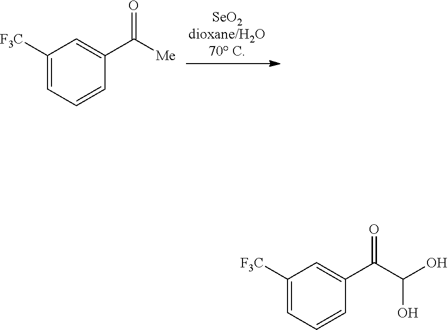 [SMILES: CC(=O)c1cccc(C(F)(F)F)c1 ; O=C(c1cccc(C(F)(F)F)c1)C(O)O]

### 2,2-dihydroxy-1-(3-(trifluoromethyl)phenyl)ethan-1-one

3′-(Trifluoromethyl)acetophenone (20.0 g, 104 mmol, 1.0 eq.) and selenium dioxide (13.9 g, 125 mmol, 1.2 eq.) were dissolved in dioxane (120 mL) and water (20.0 mL), and the mixture was stirred for 16 hours at 70° C. After this time, the reaction was cooled to room temperature and was filtered through celite. The celite pad was washed with dichloromethane and methanol. The collected filtrate was then evaporated under vacuum. To the resulting oil was added water (120 mL) and DCM (200 mL). The layers were separated, and the water layer was additionally extracted with ethyl acetate (200 mL). The organic layers were combined, dried over sodium sulfate, filtered, and concentrated to give 2,2-dihydroxy-1-(3-(trifluoromethyl)phenyl)ethan-1-one (12.0 g, 54.4 mmol, 52%) as an orange oil. The compound was used in the next step without further purification or characterization.

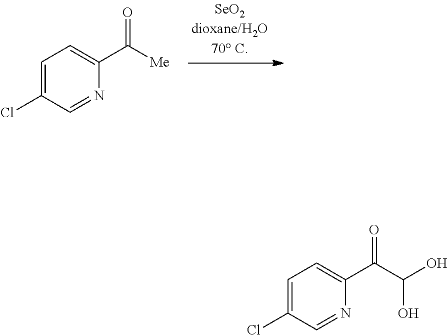 [SMILES: CC(=O)c1ccc(Cl)cn1 ; O=C(c1ccc(Cl)cn1)C(O)O]

### 1-(5-chloropyridin-2-yl)-2,2-dihydroxyethan-1-one

A mixture of 1-(5-Chloropyridin-2-yl)ethanone (5.5 g, 35.4 mmol, 1.0 eq) and selenium dioxide (4.71 g, 42.4 mmol, 1.2 eq) in a mixture dioxane (33.0 mL) and water (5.5 mL) was stirred for 16 hours at 70° C. The mixture was cooled to room temperature and filtered through celite. The celite pad was washed with dichloromethane and methanol. The combined organic solutions were evaporated under vacuum. The crude material was purified by silica gel column chromatography (0-2% MeOH in DCM) to give 1-(5-chloropyridin-2-yl)-2,2-dihydroxyethan-1-one (2.86 g, 15.2 mmol, 32%) as a brown solid. The compound was used in the next step without further purification or characterization.

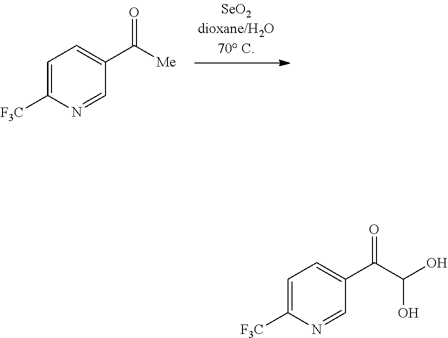 [SMILES: CC(=O)c1ccc(C(F)(F)F)nc1 ; O=C(c1ccc(C(F)(F)F)nc1)C(O)O]

### 2,2-dihydroxy-1-[6-(trifluoromethyl)pyridin-3-yl]ethan-1-one

1-[6-(Trifluoromethyl)pyridin-3-yl]ethanone (40.8 g, 211 mmol, 1.0 eq) and selenium dioxide (46.9 g, 423 mmol, 2.0 eq) in a mixture of dioxane (240 mL) and water (40.0 mL) was stirred for 16 hours at 70° C. The mixture was cooled to room temperature and filtered through celite. The celite pad was washed with dichloromethane and methanol. The combined organic solutions were evaporated under vacuum. The crude material was purified by silica gel column chromatography (0-10% MeOH in DCM) to give 2,2-dihydroxy-1-[6-(trifluoromethyl)pyridin-3-yl]ethan-1-one (8.5 g, 38.4 mmol, 16%). The compound was used in the next step without further purification or characterization.

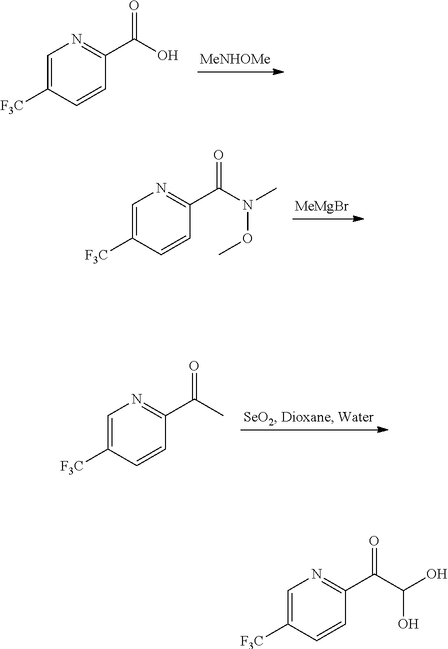

### N-methoxy-N-methyl-5-(trifluoromethyl)pyridine-2-carboxamide

Thionyl chloride (19.1 mL, 262 mmol, 10.0 eq) was added to 5-(trifluoromethyl)pyridine-2-carboxylic acid (5.0 g, 26.2 mmol, 1.0 eq), and the mixture was stirred at 80° C. for 3 hours. Upon cooling, the thionyl chloride was removed under reduced pressure, and the obtained oil was diluted with anhydrous DCM (100 mL) and cooled to −78° C. under argon atmosphere. Triethylamine (11.0 mL, 78.5 mmol, 3.0 eq) was added followed by methoxy(methyl)amine hydrochloride (3.06 g, 31.4 mmol, 1.2 eq). The reaction mixture was allowed to slowly warm to room temperature and stirred at this temperature for 16 hours. The reaction mixture was washed with 1 M aqueous HCl, saturated aqueous NaHCO₃, and brine. The organic layer was dried over sodium sulfate and concentrated to give N-methoxy-N-methyl-5-(trifluoromethyl)pyridine-2-carboxamide (5.82 g, 24.9 mmol, 92%) as dark brown oil.

UPLC-MS: RT=2.25 min, [M+H]⁺=234.90, 97% purity.

### 1-[5-(trifluoromethyl)pyridin-2-yl]ethan-1-one

N-Methoxy-N-methyl-5-(trifluoromethyl)pyridine-2-carboxamide (7.3 g, 31.2 mmol, 1.0 eq) was dissolved in anhydrous THF (80.3 mL). The reaction mixture was purged with argon, cooled to 0° C., and mixed with methylmagnesium bromide (3.0 M in diethyl ether, 13.7 mL, 41.1 mmol, 1.32 eq). The reaction mixture was allowed to warm to room temperature and stirred for 2 hours. The reaction mixture was quenched with 1 M aqueous HCl (50 mL) and extracted 3 times with EtOAc. The organic layers were collected, washed with brine, dried over sodium sulfate, and concentrated to give 1-[5-(trifluoromethyl)pyridin-2-yl]ethan-1-one (5.02 g, 22.8 mmol, 73%).

UPLC-MS: RT=2.84 min, [M+H]⁺=189.95, 86% purity.

### 2,2-dihydroxy-1-[5-(trifluoromethyl)pyridin-2-yl]ethan-1-one

A mixture of 1-[5-(trifluoromethyl)pyridin-2-yl]ethan-1-one (5.02 g, 22.8 mmol, 1.0 eq) and selenium dioxide (5.07 g, 45.7 mmol, 2.0 eq) in dioxane (30.1 mL) and water (5.02 mL) was stirred for 16 hours at 70° C. The reaction mixture was cooled to room temperature and filtered through a pad of celite, which was then washed with DCM and EtOAc. The combined solvents were removed under reduced pressure. The obtained crude material was purified via silica gel column chromatography (0-100% EtOAc in hexanes) to give 2,2-dihydroxy-1-[5-(trifluoromethyl)pyridin-2-yl]ethan-1-one (1.33 g, 6.01 mmol, 26%). This material was used directly in subsequent reactions.

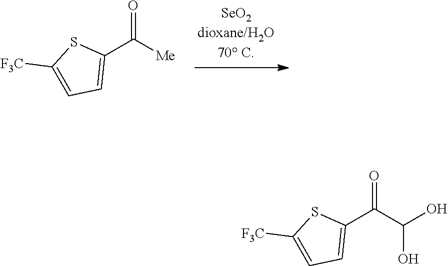 [SMILES: CC(=O)c1ccc(C(F)(F)F)s1 ; O=C(c1ccc(C(F)(F)F)s1)C(O)O]

### 2,2-dihydroxy-1-[5-(trifluoromethyl)thiophen-2-yl]ethan-1-one

1-[5-(Trifluoromethyl)thiophen-2-yl]ethan-1-one (2.5 g, 12.9 mmol, 1.0 eq) and selenium dioxide (2.86 g, 25.8 mmol, 2.0 eq) were dissolved in a mixture of dioxane (15.0 mL) and water (2.5 mL). Then, the mixture was stirred for 18 hours at 70° C. The reaction mixture was cooled to room temperature and filtered through a pad of celite. The celite pad was washed with dichloromethane. The combined organic solution was concentrated under reduced pressure. The crude material was purified by silica gel column chromatography (0-50% EtOAc in heptane) to give 2,2-dihydroxy-1-[5-(trifluoromethyl)thiophen-2-yl]ethan-1-one (2.15 g, 9.51 mmol, 63%). The compound was used in the next step without further purification or characterization.

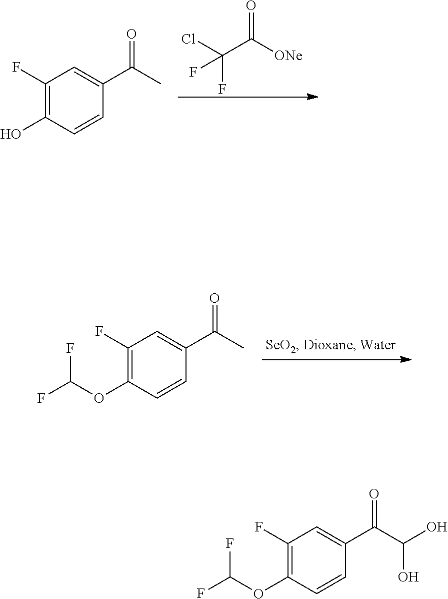

### 1-[4-(difluoromethoxy)-3-fluorophenyl]ethan-1-one

In a reaction flask under an inert atmosphere of argon, were placed 3′-fluoro-4′-hydroxyacetophenone (5.0 g, 32.4 mmol, 1.0 eq), sodium chlorodifluoroacetate (7.42 g, 48.7 mmol, 1.5 eq), and cesium carbonate (21.1 g, 64.9 mmol, 2.0 eq) in anhydrous dimethylformamide (50.0 mL). The reaction vessel was equipped with an outlet for CO₂ release. The reaction was stirred at 120° C. in an oil bath for 16 hours. The resulting mixture was diluted with ethyl acetate (100 mL) and washed with brine (3×100 mL). The organic layer was dried over anhydrous sodium sulfate and concentrated under reduced pressure. The residue was purified by silica gel flash chromatography (100% DCM) to give 1-[4-(difluoromethoxy)-3-fluorophenyl]ethan-1-one (2.9 g, 14.2 mmol, 44%).

¹H NMR (300 MHz, DMSO-d₆) δ 7.94 (dd, J=11.4, 2.1 Hz, 1H), 7.87 (ddd, J=8.5, 2.1, 1.1 Hz, 1H), 7.60-7.46 (m, 1H), 7.41 (t, J=72.7 Hz, 1H), 2.59 (s, 3H).

### 1-(4-(difluoromethoxy)-3-fluorophenyl)-2,2-dihydroxyethan-1-one

1-[4-(difluoromethoxy)-3-fluorophenyl]ethan-1-one (2.9 g, 14.2 mmol, 1.0 eq) was dissolved in dioxane (17.4 mL) and water (2.9 mL). Selenium dioxide (3.15 g, 28.4 mmol, 2.0 eq) was added. The reaction was stirred at 70° C. for 16 hours. The mixture was then cooled to room temperature and filtered through celite. The celite pad was washed with dichloromethane and methanol. The combined organic solutions were evaporated under vacuum. To the resulting oil was added water and the mixture was refluxed for 16 hours. After that time, the mixture was cooled in the fridge for 1 hour. The resulting precipitate was collected by filtration and washed with water. The aqueous filtrate was heated to reflux for 1 hour and the resulting precipitate was collected by vacuum filtration after cooling. This was repeated 4 times and all the collected precipitates were combined to give 1-(4-(difluoromethoxy)-3-fluorophenyl)-2,2-dihydroxyethan-1-one (1.2 g, 5.08 mmol, 36%) as a solid. This material was used directly in subsequent reactions.

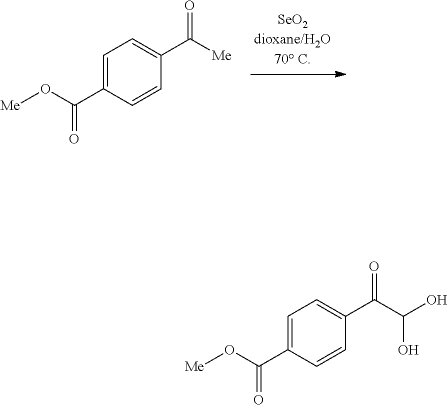 [SMILES: COC(=O)c1ccc(C(C)=O)cc1 ; COC(=O)c1ccc(C(=O)C(O)O)cc1]

### Methyl 4-(2,2-dihydroxyacetyl)benzoate

A mixture of methyl 4-acetylbenzoate (2.0 g, 11.2 mmol, 1.0 eq) and selenium dioxide (1.50 g, 13.5 mmol, 1.2 eq) in dioxane (12.0 mL) and water (2.0 mL) was stirred for 16 hours at 70° C. The reaction mixture was cooled to room temperature and filtered through celite. The celite pad was washed with dichloromethane and methanol. The combined organic solutions was concentrated in vacuo. The residue was diluted with 15 mL of water and the mixture was refluxed for 1 h. The water was then decanted and cooled in an ice bath. The precipitated product was filtered off and dried under vacuum to give methyl 4-(2,2-dihydroxyacetyl)benzoate (1.25 g, 5.95 mmol, 53%) as white solid.

¹H NMR (300 MHz, DMSO-d₆) δ 9.49 (s, 0.5H), 8.23-8.04 (m, 8H), 6.92 (d, J=7.1 Hz, 2H), 5.67 (t, J=6.8 Hz, 1H), 3.90 (d, J=3.1 Hz, 6H).

3-Chlorophenylglyoxal hydrate and 4-Fluorophenylglyoxal hydrate obtained commercially.

Synthesis of Compound 764 from Compound 336.

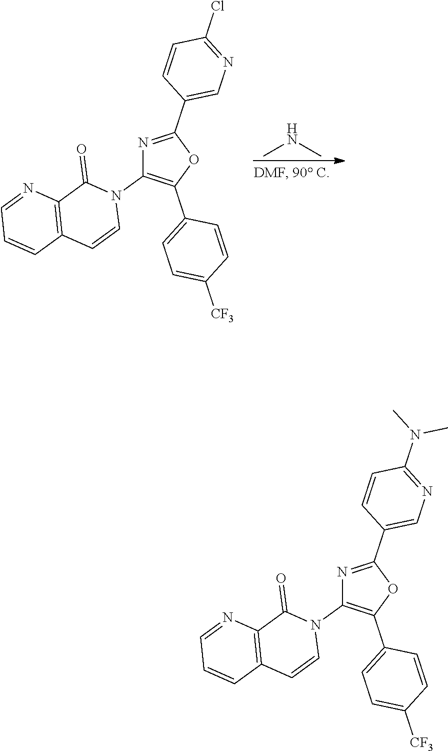

### 7-(2-(6-(dimethylamino)pyridin-3-yl)-5-(4-(trifluoromethyl)phenyl)oxazol-4-yl)-1,7-naphthyridin-8(7H)-one. Compound 764

To a solution of 7-(2-(6-chloropyridin-3-yl)-5-(4-(trifluoromethyl)phenyl)oxazol-4-yl)-1,7-naphthyridin-8(7H)-one hydrochloride (60 mg, 1 Eq, 0.12 mmol) in DMF (1 mL) was added a solution of dimethylamine (27 mg, 0.59 mL, 1.0 molar in THF, 5 Eq, 0.59 mmol), and the reaction mixture was stirred at 90° C. for 16 hours. At this time, the reaction was cooled to room temperature and concentrated to dryness. The crude product was purified by flash column chromatography (SiO₂, 0-10% MeOH in DCM) and dried under vacuum at 50° C. overnight to give 7-(2-(6-(dimethylamino)pyridin-3-yl)-5-(4-(trifluoromethyl)phenyl)oxazol-4-yl)-1,7-naphthyridin-8(7H)-one (39.9 mg, 83.6 μmol, 70%) as brown solid.

¹H NMR (400 MHz, DMSO-d₆) d ppm 8.87 (d, J=2.45 Hz, 2H), 8.26 (d, J=8.07 Hz, 1H), 8.16 (dd, J=9.05, 2.20 Hz, 1H), 7.78-7.84 (m, 3H), 7.64-7.75 (m, 3H), 6.87 (d, J=7.34 Hz, 1H), 6.82 (d, J=9.05 Hz, 1H), 3.15 (s, 6H).

¹⁹F NMR (376 MHz, DMSO-d₆) d ppm −61.24 (s, 3F).

### Synthesis of Naphthyridone Replacements

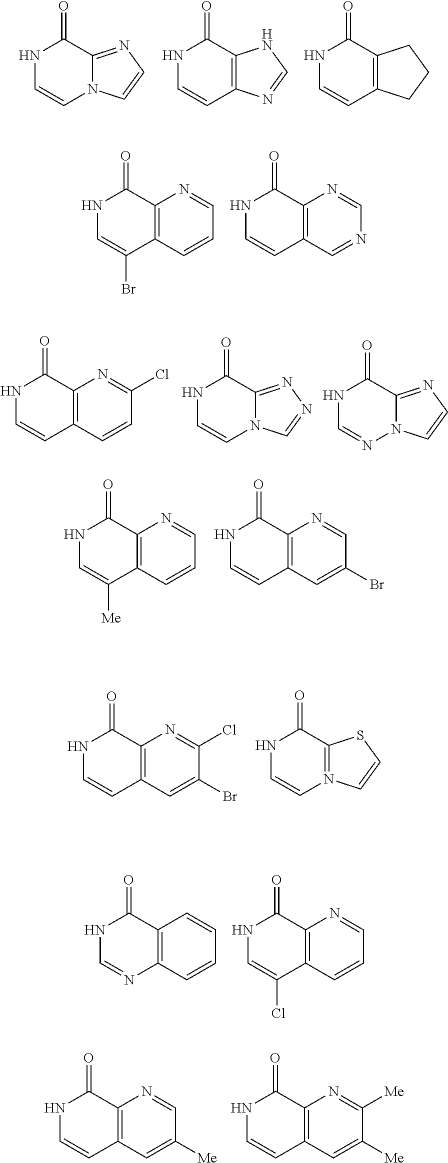

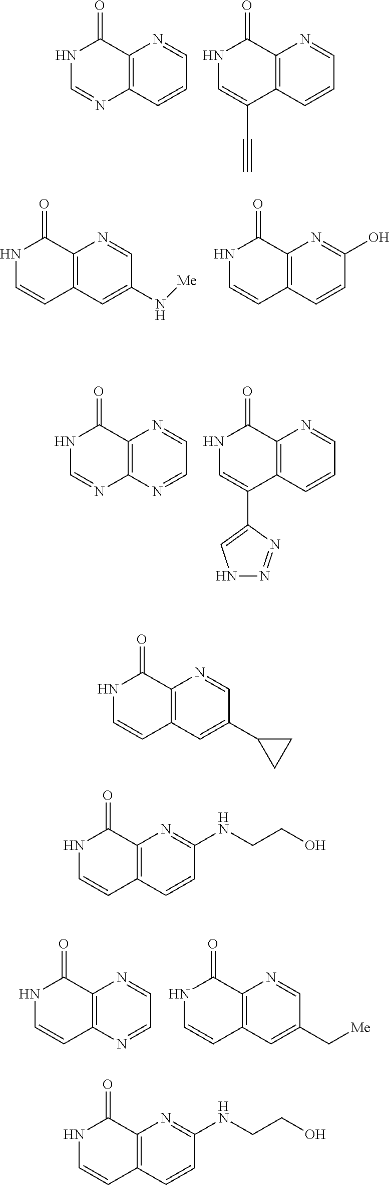

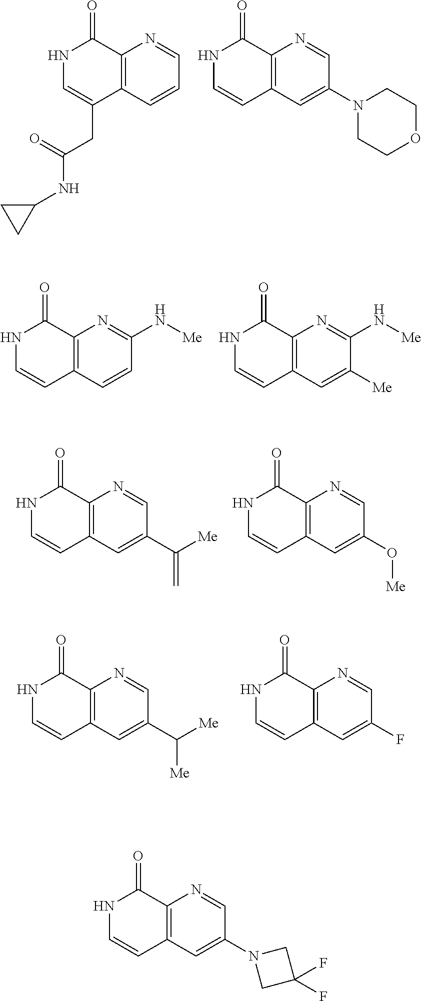

### Synthesis of Compound 765.

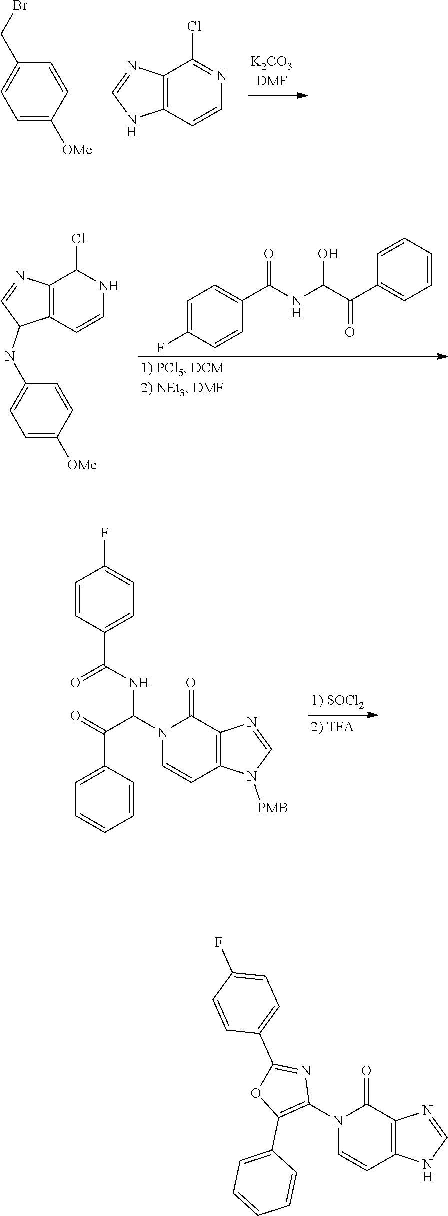

### 4-chloro-1-(4-methoxybenzyl)-1H-imidazo[4,5-c]pyridine

To a solution of 4-chloro-3H-imidazo[4,5-c]pyridine (1.00 g, 6.51 mmol, 1 eq) in N,N-dimethylformamide (25 mL) were added potassium carbonate (1.79 g, 13.0 mmol, 2 eq) and 1-(chloromethyl)-4-methoxybenzene (1.22 g, 7.81 mmol, 1.2 eq), and the mixture was stirred at room temperature for 13 hours. At this time, the reaction was treated with water (30 mL) and extracted with EtOAc (2×50 mL). The combined organic layers were washed with brine, dried over MgSO₄, filtered, concentrated, and dried to give 4-chloro-3-(4-methoxybenzyl)-3H-imidazo[4,5-c]pyridine (1.20 g, 67.4%) as a white foam.

¹H NMR (400 MHz, CHLOROFORM-d) d ppm 3.80 (s, 2H) 5.68 (s, 2H) 6.89 (d, J=8.56 Hz, 1H) 7.15 (d, J=8.56 Hz, 1H) 7.62-7.72 (m, 1H) 7.98 (s, 1H) 8.17-8.27 (m, 1H).

LCMS: rt 1.94 min. [M+H]⁺ 274.1 m/z.

### 1-(4-methoxybenzyl)-1,5-dihydro-4H-imidazo[4,5-c]pyridin-4-one

To a solution of 4-chloro-3-(4-methoxybenzyl)-3H-imidazo[4,5-c]pyridine (0.180 g, 0.658 mmol, 1 eq) in dioxane (2 mL) was added hydrogen chloride (4 M in dioxane, 0.82 mL, 3.28 mmol, 5 eq), and the reaction was heated to 110° C. for 24 hours. The reaction was cooled to room temperature, concentrated, and dried to give 3-(4-methoxybenzyl)-3H-imidazo[4,5-c]pyridin-4-ol (90.0 mg, 53.8%) as a brown solid.

LCMS (ESI+): m/z 256.1, [M+H]⁺.

### 4-fluoro-N-(1-(1-(4-methoxybenzyl)-4-oxo-1,4-dihydro-5H-imidazo[4,5-c]pyridin-5-yl)-2-oxo-2-phenylethyl)benzamide

A mixture of 4-fluoro-N-(1-hydroxy-2-oxo-2-phenylethyl)benzamide (12.8 g, 46.4 mmol, 1 eq) and phosphorous pentachloride (10.1 g, 48.5 mmol, 1.05 eq) in tetrachloromethane (200 mL) was heated at 60° C. for 3 hours. After cooling to room temperature, the generated precipitate was collected vacuum filtration to give N-(1-chloro-2-oxo-2-phenylethyl)-4-fluorobenzamide (9.00 g, 66.6%) as a white solid. To a solution of 3-(4-methoxybenzyl)-3H-imidazo[4,5-c]pyridin-4-ol (144 mg, 0.566 mmol, 1.1 eq) in N,N-dimethylformamide (5 mL) was added triethylamine (181 mg, 1.79 mmol, 3.5 eq) and N-(1-chloro-2-oxo-2-phenylethyl)-4-fluorobenzamide (150 mg, 0.514 mmol, 1 eq). The mixture was stirred at room temperature for 2 hours. The reaction was treated with water (10 mL), and the resulting precipitate was collected by vacuum filtration and washed with water. The product was dried under vacuum at 50° C. to give 4-fluoro-N-(1-(3-(4-methoxybenzyl)-4-oxo-3H-imidazo[4,5-c]pyridin-5(4H)-yl)-2-oxo-2-phenylethyl)benzamide (232 mg, 88.5%) as a white solid.

LCMS (ESI+): m/z 511.3, [M+H]⁺.

### 5-(2-(4-fluorophenyl)-5-phenyloxazol-4-yl)-3H-imidazo[4,5-c]pyridin-4(5H)-one. Compound 765

To 4-fluoro-N-(1-(3-(4-methoxybenzyl)-4-oxo-3H-imidazo[4,5-c]pyridin-5(4H)-yl)-2-oxo-2-phenylethyl)benzamide (122 mg, 0.239 mmol, 1 eq) was added thionyl chloride (908 mg, 7.64 mmol, 32 eq), and the reaction mixture was heated to 80° C. for 2 hours. The reaction was cooled to 0° C. and quenched with ice water (3 mL). The ice bath was removed, and the resulting slurry was stirred at room temperature for 30 minutes. The resulting precipitate was collected by vacuum filtration, washed with water, and dried under vacuum to give 5-(2-(4-fluorophenyl)-5-phenyloxazol-4-yl)-3-(4-methoxybenzyl)-3H-imidazo[4,5-c]pyridin-4(5H)-one hydrochloride (102 mg, 80.9%) as a white solid. To 5-(2-(4-fluorophenyl)-5-phenyloxazol-4-yl)-3-(4-methoxybenzyl)-3H-imidazo[4,5-c]pyridin-4(5H)-one hydrochloride (33.0 mg, 0.0624 mmol) was added 1 mL of TFA, and the reaction mixture was heated to 80° C. for 2 hours. The reaction was then cooled to room temperature and concentrated. The resulting black residue was purified by reverse phase HPLC (C18, 10-100% MeCN in water with 0.1% TFA) to give 5-(2-(4-fluorophenyl)-5-phenyloxazol-4-yl)-3H-imidazo[4,5-c]pyridin-4(5H)-one (12.1 mg, 47.6%) as a

¹H NMR (400 MHz, DMSO-d₆) d ppm 6.90 (d, J=7.34 Hz, 2H) 7.35-7.56 (m, 12H) 7.67 (d, J=7.34 Hz, 2H) 8.21 (dd, J=8.80, 5.38 Hz, 3H) 8.68 (s, 1H).

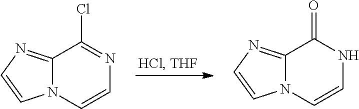 [SMILES: Clc1nccn2ccnc12 ; O=c1[nH]ccn2ccnc12]

### Imidazo[1,2-a]pyrazin-8(7H)-one hydrochloride

To a solution of 8-chloroimidazo[1,2-a]pyrazine (300 mg, 1.95 mmol, 1 eq) in dioxane (7 mL) was added a 6 N solution of aqueous hydrogen chloride (1.06 g, 29.2 mmol, 15 eq) and heated to 80° C. for 16 hours. After cooling to room temperature, the reaction was concentrated to dryness. The material was dried under vacuum at 50° C. to give imidazo[1,2-c]pyrazin-8(7H)-one hydrochloride (270 mg, 80.8%) as a pale yellow solid.

LCMS (ESI+): m/z 136.3, [M+H]⁺.

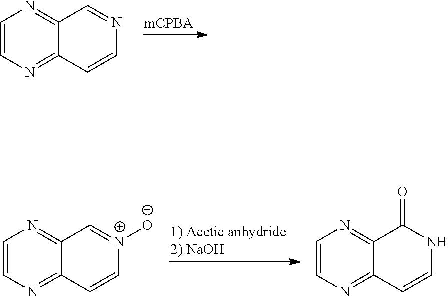 [SMILES: c1cc2nccnc2cn1 ; [O-][n+]1ccc2nccnc2c1 ; O=c1[nH]ccc2nccnc12]

### pyrido[3,4-b]pyrazine 6-oxide

To a solution of pyrido[3,4-b]pyrazine (500 mg, 1 Eq, 3.81 mmol) in CH₂Cl₂ (20 mL) was added 3-chlorobenzoperoxoic acid (1.0 g, 70 Wt %, 1.08 Eq, 4.12 mmol), and the reaction mixture was stirred at room temperature for 18 hours. The reaction mixture was concentrated in vacuo. The residue was purified by flash column chromatography (silica gel, 0-10% MeOH in DCM) to give pyrido[3,4-b]pyrazine 6-oxide (499 mg, 3.39 mmol, 88.9%) as a yellow solid.

¹H NMR: (400 MHz, CHLOROFORM-d) d ppm 7.97 (d, J=7.34 Hz, 1H) 8.39 (dd, J=7.34, 1.96 Hz, 1H) 8.87 (d, J=1.47 Hz, 1H) 8.92 (d, J=1.47 Hz, 1H) 9.00 (d, J=1.71 Hz, 1H).

### Pyrido[3,4-b]pyrazin-5(6H)-one

Pyrido[3,4-b]pyrazine 6-oxide (499 mg, 1 Eq, 3.39 mmol) and acetic anhydride (37.4 g, 34.6 mL, 108 Eq, 366 mmol) were mixed in a round bottom flask, and the mixture was heated at 140° C. for 18 hours. The reaction mixture was cooled to room temperature and concentrated in vacuo. The residue was dissolved in sodium hydroxide (0.8 g, 20.7 mL, 1 molar, 6 Eq, 20.7 mmol) and heated at 80° C. for 2 hours. After cooling, the reaction mixture was concentrated in vacuo. The residue was purified by flash column chromatography (silica gel, 0-10% MeOH in DCM) to give pyrido[3,4-b]pyrazin-5(6H)-one (122 mg, 831 μmol, 24.5%) as a brown solid.

¹H NMR: (400 MHz, DMSO-d6) d ppm 6.64 (d, J=7.34 Hz, 1H), 7.53 (d, J=5.87 Hz, 1H), 8.80 (d, J=1.96 Hz, 1H), 8.95 (d, J=1.96 Hz, 1H), 11.85 (brs, 1H).

Additional Compounds were Prepared in a Manner Analogous to Compound 479 Via Final Cyclization in Thionyl Chloride:

| | Exact | | |
|---|---|---|---|
| | mass | | LCMS |
| Compound | (g/mol) | LCMS | Assignment |
| 766 | 355.1069 | 356.1 | [M + H]+ |
| 767 | 365.1164 | | [M + H]+ |
| 768 | 366.1117 | 367.0 | |
| 769 | 366.1117 | 367.1 | [M + H]+ |
| 770 | 366.117 | 367.1 | [M + H]+ |
| 771 | 367.1069 | 368.1 | [M + H]+ |
| 772 | 371.0728 | 372.0 | [M + H]+ |
| 773 | 373.0975 | 374.0 | [M + H]+ |
| 775 | 433.0385 | 434.0 | [M + H]+ |
| 776 | 443.0269 | 444.0 | [M + H]+ |
| 777 | 443.0269 | 444.0 | [M + H]+ |

### Synthesis of Compound 774.

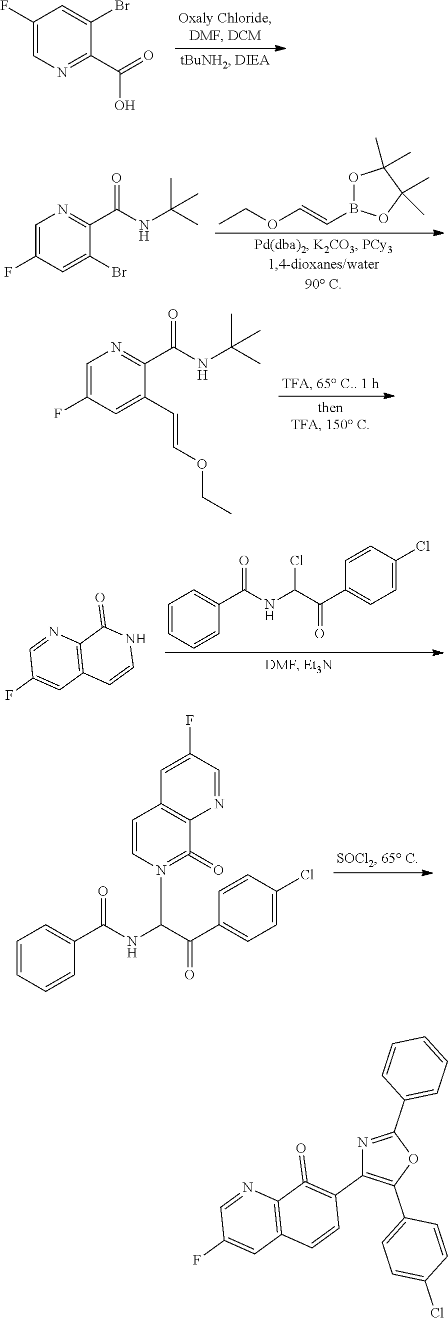

### 3-bromo-N-(tert-butyl)-5-fluoropicolinamide

To a 0° C. slurry of 3-bromo-5-fluoropicolinic acid (5.48 g, 24.9 mmol, 1 eq) in DCM (100 mL) was added oxalyl dichloride (3.32 g, 26.2 mmol, 1.05 Eq) followed by N,N-dimethylformamide (91.0 mg, 1.25 mmol, 0.05 Eq) dropwise. The cold bath was removed, and the reaction was stirred at room temperature for 1 hour. At this time, the reaction was chilled back to 0° C. and treated with N-ethyl-N-isopropylpropan-2-amine (6.44 g, 49.8 mmol, 2.0 Eq) and tert-butylamine (2.19 g, 29.9 mmol, 1.2 Eq). The cold bath was removed, and the resulting brown solution was stirred to at room temperature over 1.5 hours. The reaction was washed with 50 mL of 0.25 N HCl. The aqueous layer was extracted with 100 mL of DCM. The combined organic layers were washed with brine, dried over MgSO₄, filtered, and concentrated to give 3-bromo-N-(tert-butyl)-5-fluoropicolinamide as light-yellow solid.

¹H NMR (400 MHz, CHLOROFORM-d) □ 8.35 (dd, J=0.49, 2.45 Hz, 1H), 7.78 (dd, J=2.45, 7.83 Hz, 1H), 7.46-7.61 (m, 1H), 1.48 (s, 9H).

¹⁹F NMR (377 MHz, CHLOROFORM-d) □ ppm −121.72 (d, J=6.81 Hz, 1F).

LCMS: rt 2.01 min. [M+H]⁺ 274.9 m/z.

### (E)-N-(tert-butyl)-3-(2-ethoxyvinyl)-5-fluoropicolinamide

3-Bromo-N-(tert-butyl)-5-fluoropicolinamide (3.3 g, 12 mmol, 1 Eq), (E)-2-(2-ethoxyvinyl)-4,4,5,5-tetramethyl-1,3,2-dioxaborolane (2.4 g, 12 mmol, 1 Eq), potassium carbonate (5.0 g, 36 mmol, 3 Eq), tricyclohexylphosphine (0.34 g, 1.2 mmol, 0.1 Eq), and bis(dibenzylideneacetone)palladium (0.34 g, 0.60 mmol, 0.05 Eq) were combined, and the reaction vessel was purged with argon. The solid mixture was charged with dioxane (70 mL) and water (7.0 mL). The reaction mixture was purged with argon for 5 minutes and heated to 90° C. for 18 hours. The reaction was cooled to room temperature, diluted with EtOAc (70 mL), and washed with brine. The aqueous was extracted with EtOAc (70 mL). The combined organic layers were dried over MgSO₄, filtered and concentrated to viscous brown oil. The crude material was purified by silica gel flash column chromatography (0-20% MeOH in DCM) to give (E)-N-(tert-butyl)-3-(2-ethoxyvinyl)-5-fluoropicolinamide (3.2 g, 12 mmol, 100%) as a beige solid.

LCMS: rt 2.42 min. [M+H]⁺ 267.1 m/z.

### 3-fluoro-1,7-naphthyridin-8(7H)-one

(E)-N-(tert-Butyl)-3-(2-ethoxyvinyl)-5-fluoropicolinamide (1.00 g, 3.75 mmol, 1 Eq) was treated with TFA (11.8 g, 104 mmol, 27.7 Eq) and heated to 65° C. for 1 hour. The reaction was then heated to 150° C. for 2 hours. The reaction was concentrated to yield a dark brown viscous oil. The crude material was triturated with wet THF and a white precipitate formed. This material was collected by vacuum filtration, washed with THF, and dried under vacuum to give 3-fluoro-1,7-naphthyridin-8(7H)-one (0.446 g, 72.4%) as a white solid.

LCMS: rt 0.37 min. [M+H]⁺ 165.0 m/z.

### N-(2-(4-chlorophenyl)-1-(3-fluoro-8-oxo-1,7-naphthyridin-7(8H)-yl)-2-oxoethyl)benzamide

N-(1-Chloro-2-(4-chlorophenyl)-2-oxoethyl)benzamide (149 mg, 484 μmol, 1 Eq) was treated with a slurry of 3-fluoro-1,7-naphthyridin-8(7H)-one (94 mg, 1.2 Eq, 0.57 mmol) and triethylamine (0.20 g, 0.28 mL, 4.1 Eq, 2.0 mmol) in DCM (3 mL), and the reaction mixture was stirred at room temperature for 3 hours. The reaction was treated with ether, and the resulting precipitate was collected by vacuum filtration, washed with water, and dried under vacuum at 50° C. to give N-(2-(4-chlorophenyl)-1-(3-fluoro-8-oxo-1,7-naphthyridin-7(8H)-yl)-2-oxoethyl)benzamide (211 mg, 484 μmol, 100%) as yellow solid.

LCMS: rt 2.24 min. [2M+H+Na]⁺ 895.0 m/z.

### 7-(5-(4-chlorophenyl)-2-phenyloxazol-4-yl)-3-fluoro-1,7-naphthyridin-8(7H)-one. Compound 774

N-(2-(4-Chlorophenyl)-1-(3-fluoro-8-oxo-1,7-naphthyridin-7(8H)-yl)-2-oxoethyl)benzamide (0.211 g, 1 Eq, 484 μmol) was treated with thionyl chloride (3.3 g, 2.0 mL, 57 Eq, 27 mmol), and the reaction mixture was heated to 65° C. for 3 hours. The reaction was cooled to room temperature and concentrated to a dark brown viscous oil. The crude product was triturated with DCM/Et₂O and collected by vacuum filtration. This material was washed with Et₂O and dried under vacuum at 50° C. to give 7-(5-(4-chlorophenyl)-2-phenyloxazol-4-yl)-3-fluoro-1,7-naphthyridin-8(7H)-one (150 mg, 359 μmol, 74.2%) as a brown solid.

¹H NMR (400 MHz, CHLOROFORM-d) d ppm 6.59 (d, J=6.85 Hz, 1H), 7.30-7.43 (m, 3H), 7.45-7.56 (m, 5H), 7.61 (d, J=7.58 Hz, 1H), 8.05-8.16 (m, 2H), 8.80 (brs, 1H).

LCMS: rt 2.50 min. [M+H]⁺ 418.0 m/z.

### Synthesis of Compound 778.

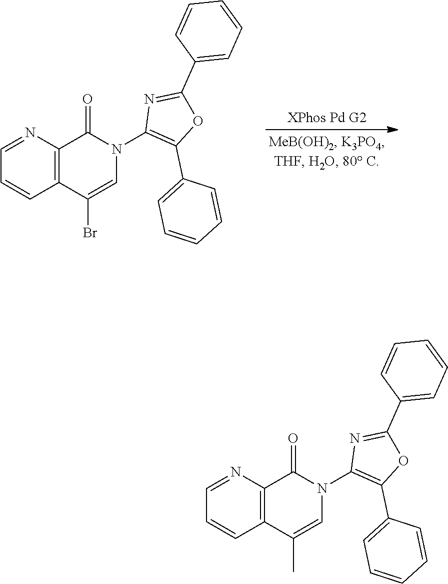 [SMILES: O=c1c2ncccc2c(Br)cn1-c1nc(-c2ccccc2)oc1-c1ccccc1 ; Cc1cn(-c2nc(-c3ccccc3)oc2-c2ccccc2)c(=O)c2ncccc12]

### 7-(2,5-diphenyloxazol-4-yl)-5-methyl-1,7-naphthyridin-8(7H)-one. Compound 778

To the solution of 5-bromo-7-(2,5-diphenyloxazol-4-yl)-1,7-naphthyridin-8(7H)-one (21.0 mg, 1 Eq, 47.3 μmol) in 1,4-dioxane (2 mL) and water (0.5 mL) were added potassium phosphate (30.1 mg, 3 Eq, 142 μmol), XPhos Pd G2 (3.72 mg, 0.1 Eq, 4.73 μmol), and methylboronic acid (3.11 mg, 1.1 Eq, 52.0 μmol). The mixture was heated at 80° C. for 2 days under nitrogen atmosphere. The reaction mixture was cooled to room temperature and concentrated under reduced pressure. The residue was purified by flash column chromatography (silica gel, 100% EtOAc) to give 7-(2,5-diphenyloxazol-4-yl)-5-methyl-1,7-naphthyridin-8(7H)-one (6.1 mg, 16 lima 34%) as a white solid.

¹H NMR: (400 MHz, CHLOROFORM-d) d ppm 2.36 (s, 3H), 7.33-7.44 (m, 5H), 7.50-7.57 (m, 4H), 7.59-7.65 (m, 2H), 8.09-8.22 (m, 3H).

LCMS: rt 2.37 min. [M+H]⁺ 380.1 m/z.

### Synthesis of Compound 779.

### 7-(2,5-diphenyloxazol-4-yl)-3-methyl-1,7-naphthyridin-8(7H)-one. Compound 779

To a solution of 3-bromo-7-(2,5-diphenyloxazol-4-yl)-1,7-naphthyridin-8(7H)-one (60 mg, 1 Eq, 0.14 mmol) in 1,4-dioxane (4 mL) and water (1 mL) were added potassium phosphate (86 mg, 3 Eq, 0.41 mmol), XPhos Pd G2 (11 mg, 0.1 Eq, 14 μmol), and methylboronic acid (12 mg, 1.5 Eq, 0.20 mmol). The mixture was heated at 80° C. for 18 hours under nitrogen atmosphere. The reaction mixture was cooled to room temperature and concentrated under reduced pressure. The residue was purified by flash column chromatography (silica gel, 100% EtOAc) to give 7-(2,5-diphenyloxazol-4-yl)-3-methyl-1,7-naphthyridin-8(7H)-one (24 mg, 63 μmol, 47%) as a pale-yellow solid.

¹H NMR: (400 MHz, CHLOROFORM-d) d ppm 2.50-2.67 (m, 3H), 6.55 (d, J=7.34 Hz, 1H), 7.32-7.43 (m, 3H), 7.47-7.65 (m, 6H), 7.77 (s, 1H), 8.15 (dd, J=6.60, 2.93 Hz, 2H), 8.82 (s, 1H).

LCMS: rt 2.33 min. [M+H]⁺ 380.1 m/z.

Synthesis of Compound 780.

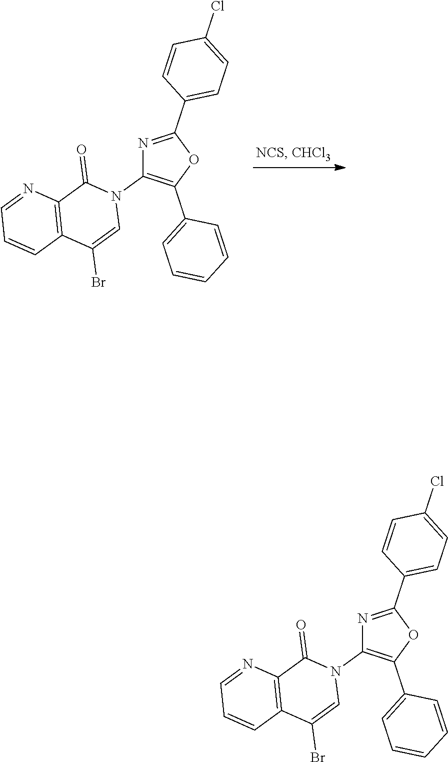 [SMILES: O=c1c2ncccc2c(Br)cn1-c1nc(-c2ccc(Cl)cc2)oc1-c1ccccc1 ; O=c1c2ncccc2c(Br)cn1-c1nc(-c2ccc(Cl)cc2)oc1-c1ccccc1]

### 5-chloro-7-(2-(4-chlorophenyl)-5-phenyloxazol-4-yl)-1,7-naphthyridin-8(7H)-one. Compound 780

To a solution of 7-(2-(4-chlorophenyl)-5-phenyloxazol-4-yl)-1,7-naphthyridin-8(7H)-one (102 mg, 1 Eq, 255 μmol) in CHCl₃ (2 mL) was added NCS (40.9 mg, 1.2 Eq, 306 μmol), and the reaction mixture was heated to 50° C. After 19 hours, additional NCS (40.9 mg, 1.2 Eq, 306 μmol) was added, and the mixture was stirred at 65° C. for an additional 7 hours. After cooling, the reaction was concentrated and purified by flash column chromatography (SiO₂, 0-10% MeOH in DCM) to give 5-chloro-7-(2-(4-chlorophenyl)-5-phenyloxazol-4-yl)-1,7-naphthyridin-8(7H)-one (28.2 mg, 64.9 μmol, 25.5%) as light brown solid.

¹H NMR (400 MHz, DMSO-d₆) d ppm 7.38-7.51 (m, 3H), 7.59-7.65 (m, 2H), 7.69 (d, J=8.80 Hz, 2H), 7.95-8.01 (m, 1H), 8.17 (d, J=8.80 Hz, 2H), 8.18 (s, 1H), 8.34-8.43 (m, 1H), 8.94-9.02 (m, 1H).

LCMS: rt 2.77 min. [M+H]⁺ 433.9 m/z.

### Synthesis of Compound 781.

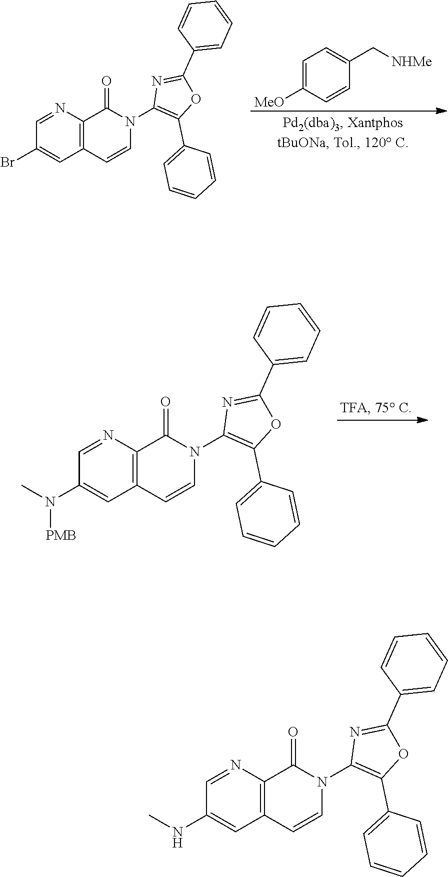

### 7-(2,5-diphenyloxazol-4-yl)-3-((4-methoxybenzyl)(methyl)amino)-1,7-naphthyridin-8(7H)-one

To a microwave reactor vial were added 3-bromo-7-(2,5-diphenyloxazol-4-yl)-1,7-naphthyridin-8(7H)-one (150 mg, 1 Eq, 338 μmol), toluene (1 mL), 4,5-bis(diphenylphosphino)-9,9-dimethyl xanthene (58.6 mg, 0.3 Eq, 101 μmol), Pd₂(dba)₃ (30.9 mg, 0.1 Eq, 33.8 μmol), (4-methoxybenzyl)methylamine (56.2 mg, 55.7 μL, 1.1 Eq, 371 μmol), and sodium 2-methylpropan-2-olate (48.7 mg, 1.5 Eq, 506 μmol). The reaction mixture was then irradiated in the microwave at 120° C. for 1 hour. Upon cooling, the reaction mixture was filtered and concentrated in vacuo. The residue was purified by silica gel flash column chromatography (5% MeOH in EtOAc) to give 7-(2,5-diphenyloxazol-4-yl)-3-((4(4-methoxybenzyl)(methyl)amino)-1,7-naphthyridin-8(7H)-one (70.5 mg, 137 μmol, 40.6%) as a pale-yellow solid.

¹H NMR: (400 MHz, CHLOROFORM-d) d ppm 3.24 (s, 3H), 3.83 (s, 3H), 4.70 (s, 2H), 6.42 (d, J=7.34 Hz, 1H), 6.88-6.94 (m, 3H), 7.18 (dd, J=7.95, 4.03 Hz, 3H), 7.32-7.41 (m, 4H), 7.49-7.55 (m, 3H), 7.62 (d, J=7.09 Hz, 2H), 8.14 (dd, J=6.48, 2.81 Hz, 2H), 8.63 (d, J=2.45 Hz, 1H).

LCMS: rt 2.57 min. [M+H]⁺ 515.2 m/z

### 7-(2,5-diphenyloxazol-4-yl)-3-(methylamino)-1,7-naphthyridin-8(7H)-one. Compound 781

7-(2,5-Diphenyloxazol-4-yl)-3-((4(4-methoxybenzyl)(methyl)amino)-1,7-naphthyridin-8(7H)-one (70.5 mg, 1 Eq, 137 μmol) and TFA (1 mL) were combined and stirred at 75° C. for 5 hours. The reaction mixture was then cooled and concentrated. The residue was neutralized with saturated aqueous NaHCO₃ and extracted with DCM. The organic solution was washed with brine, dried over MgSO₄ and concentrated in vacuo. The residue was triturated with heptane, filtered, and dried in vacuo to give 7-(2,5-diphenyloxazol-4-yl)-3-(methylamino)-1,7-naphthyridin-8(7H)-one (36.3 mg, 92.0 μmol, 67.2%) as pale-yellow solid.

¹H NMR: (400 MHz, CHLOROFORM-d) d ppm 3.01 (s, 3H), 6.47 (d, J=7.58 Hz, 1H), 6.80 (s, 1H), 7.20 (d, J=7.34 Hz, 1H), 7.31-7.45 (m, 4H), 7.52 (d, J=2.93 Hz, 3H), 7.61 (d, J=7.34 Hz, 2H), 8.14 (d, J=3.42 Hz, 2H), 8.44 (brs, 1H).

LCMS: rt 2.23 min. [M+H]⁺ 395.1 m/z.

### Synthesis of Compound 782.

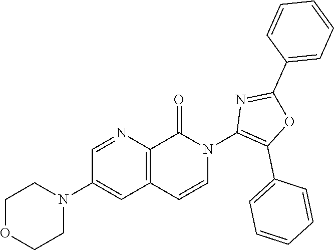 [SMILES: O=c1c2ncc(N3CCOCC3)cc2ccn1-c1nc(-c2ccccc2)oc1-c1ccccc1]

7-(2,5-diphenyloxazol-4-yl)-3-morpholino-1,7-naphthyridin-8(7H)-one. Compound 782 was prepared in a manner analogous to compound 781, without the requirement of TFA treatment.

¹H NMR: (400 MHz, CHLOROFORM-d) d ppm 3.37-3.52 (m, 4H), 3.90-4.01 (m, 4H), 6.47 (d, J=7.58 Hz, 1H), 7.09 (d, J=2.45 Hz, 1H), 7.23 (d, J=7.58 Hz, 1H), 7.32-7.43 (m, 3H), 7.49-7.56 (m, 3H), 7.61 (d, J=7.09 Hz, 2H), 8.06-8.21 (m, 2H), 8.70 (d, J=2.45 Hz, 1H).

LCMS: rt 2.29 min. [M+H]⁺ 451.1 m/z.

### Synthesis of Compound 783.

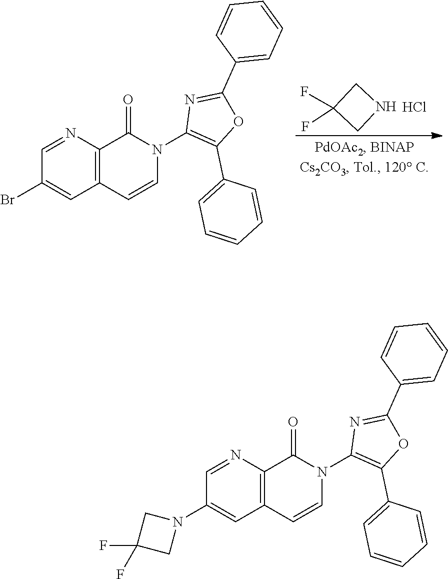 [SMILES: O=c1c2ncc(Br)cc2ccn1-c1nc(-c2ccccc2)oc1-c1ccccc1 ; FC1(F)CNC1 ; Cl ; O=c1c2ncc(N3CC(F)(F)C3)cc2ccn1-c1nc(-c2ccccc2)oc1-c1ccccc1]

### 3-(3,3-difluoroazetidin-1-yl)-7-(2,5-diphenyloxazol-4-yl)-1,7-naphthyridin-8(7H)-one. Compound 783

In a microwave vial were combined 3-bromo-7-(2,5-diphenyloxazol-4-yl)-1,7-naphthyridin-8(7H)-one (150 mg, 1 Eq, 338 μmol), toluene (2 mL), BINAP (21.0 mg, 0.1 Eq, 33.8 μmol), cesium carbonate (220 mg, 2 Eq, 675 μmol), palladium diacetate (7.58 mg, 0.1 Eq, 33.8 μmol), and 3,3-difluoroazetidine hydrochloride (48.1 mg, 1.1 Eq, 371 μmol). The mixture was then irradiated in the microwave reactor at 120° C. for 1 hour. The reaction mixture was filtered and concentrated in vacuo. The residue was purified by silica gel flash column chromatography (100% EtOAc) to give 3-(3,3-difluoroazetidin-1-yl)-7-(2,5-diphenyloxazol-4-yl)-1,7-naphthyridin-8(7H)-one (17.8 mg, 39.0 μmol, 11.6%) as a pale-yellow solid.

¹H NMR: (400 MHz, CHLOROFORM-d) d ppm 4.52 (t, J=11.62 Hz, 4H), 6.47 (d, J=7.34 Hz, 1H), 6.80 (d, J=2.45 Hz, 1H), 7.26 (d, J=7.34 Hz, 1H), 7.34-7.42 (m, 3H), 7.46-7.66 (m, 5H), 8.14 (d, J=3.42 Hz, 2H), 8.30 (d, J=2.20 Hz, 1H).

LCMS: rt 2.46 min. [M+H]⁺ 457.0 m/z.

### Synthesis of Compound 784.

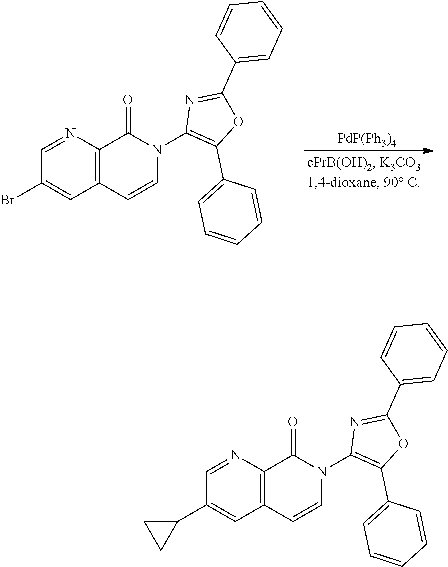 [SMILES: O=c1c2ncc(Br)cc2ccn1-c1nc(-c2ccccc2)oc1-c1ccccc1 ; O=c1c2ncc(C3CC3)cc2ccn1-c1nc(-c2ccccc2)oc1-c1ccccc1 ; "PdP(Ph3)4"]

### 3-cyclopropyl-7-(2,5-diphenyloxazol-4-yl)-1,7-naphthyridin-8(7H)-one. Compound 784

To a solution of 3-bromo-7-(2,5-diphenyloxazol-4-yl)-1,7-naphthyridin-8(7H)-one (150 mg, 1 Eq, 338 μmol) in 1,4-dioxane (2 mL) were added cyclopropylboronic acid (58.0 mg, 2 Eq, 675 μmol), potassium carbonate (140 mg, 3 Eq, 1.01 mmol), and tetrakis(triphenylphosphine)palladium(0) (46.8 mg, 0.12 Eq, 40.5 μmol). The mixture was then heated to 90° C. for 18 hours under nitrogen atmosphere. After cooling, the reaction mixture was concentrated. The residue was purified by flash silica gel column chromatography (100% EtOAc) to give 3-cyclopropyl-7-(2,5-diphenyloxazol-4-yl)-1,7-naphthyridin-8(7H)-one (80.6 mg, 199 μmol, 58.9%) as a pale-yellow solid.

¹H NMR: (400 MHz, CHLOROFORM-d) d ppm 0.85-1.03 (m, 2H), 1.24 (dd, J=8.19, 1.59 Hz, 2H), 2.07 (s, 1H), 6.52 (d, J=7.34 Hz, 1H), 7.32-7.41 (m, 2H), 7.45-7.63 (m, 4H), 7.70 (dd, J=11.98, 7.09 Hz, 4H), 8.15 (dd, J=6.60, 2.93 Hz, 2H), 8.75 (d, J=1.96 Hz, 1H).

LCMS: rt 2.46 min. [M+H]⁺ 406.1 m/z.

### Synthesis of Compound 785.

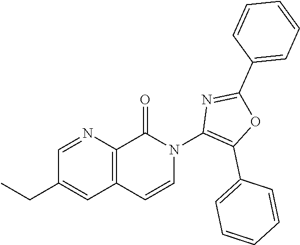 [SMILES: CCc1cnc2c(=O)n(-c3nc(-c4ccccc4)oc3-c3ccccc3)ccc2c1]

7-(2,5-diphenyloxazol-4-yl)-3-ethyl-1,7-naphthyridin-8(7H)-one. Compound 785. This was prepared in a manner analogous to 3-cyclopropyl-7-(2,5-diphenyloxazol-4-yl)-1,7-naphthyridin-8(7H)-one, compound 498.

¹H NMR: (400 MHz, CHLOROFORM-d) d ppm 1.40 (t, J=7.58 Hz, 3H), 2.88 (q, J=7.58 Hz, 2H), 6.56 (d, J=7.34 Hz, 1H), 7.32-7.42 (m, 3H), 7.46-7.64 (m, 5H), 7.70 (dd, J=11.86, 7.21 Hz, 1H), 7.75 (s, 1H), 8.15 (dd, J=6.36, 2.93 Hz, 2H), 8.83 (d, J=1.71 Hz, 1H).

LCMS: rt 2.43 min. [M+H]⁺ 394.1 m/z.

### Synthesis of Compound 786.

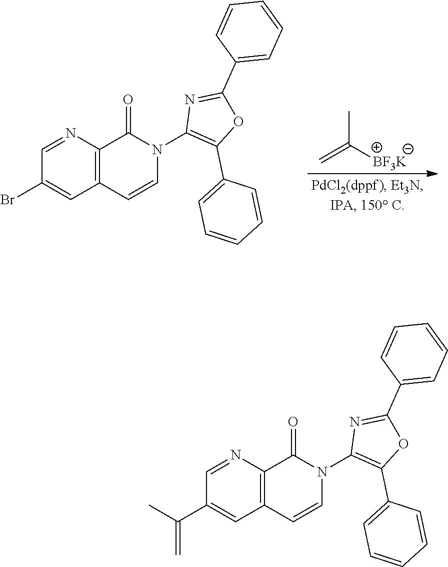

### 7-(2,5-diphenyloxazol-4-yl)-3-(prop-1-en-2-yl)-1,7-naphthyridin-8(7H)-one. Compound 786

In a microwave vial were combined 3-bromo-7-(2,5-diphenyloxazol-4-yl)-1,7-naphthyridin-8(7H)-one (120 mg, 1 Eq, 270 μmol), 2-propanol (1.5 mL), potassium trifluoro(isopropenyl)borate (48.0 mg, 1.2 Eq, 324 μmol), triethylamine (49.2 mg, 67 μL, 1.8 Eq, 486 μmol), and 1,1′-bis(diphenylphosphino)ferrocene-palladium(II) dichloride (19.8 mg, 0.1 Eq, 27.0 μmol). The mixture was sealed and irradiated in the microwave reactor at 150° C. for 30 minutes. After cooling, the reaction mixture was concentrated. The residue was purified by silica gel flash column chromatography (100% EtOAc) to give 7-(2,5-diphenyloxazol-4-yl)-3-(prop-1-en-2-yl)-1,7-naphthyridin-8(7H)-one (57.8 mg, 143 μmol, 52.8%) as a white solid.

¹H NMR: (400 MHz, CHLOROFORM-d) d ppm 2.30 (s, 3H), 5.25 (s, 1H), 6.51-6.65 (m, 2H), 7.30-7.43 (m, 4H), 7.53 (d, J=2.69 Hz, 4H), 7.61 (d, J=7.34 Hz, 2H), 8.15 (brs, 2H), 9.09 (s, 1H).

LCMS: rt 2.55 min. [M+H]⁺ 406.1 m/z.

### Synthesis of Compound 787.

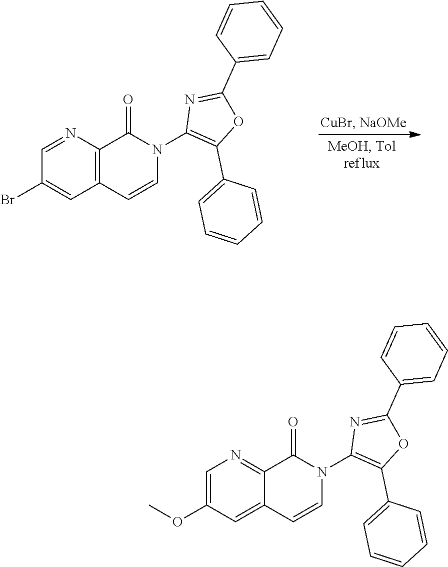 [SMILES: O=c1c2ncc(Br)cc2ccn1-c1nc(-c2ccccc2)oc1-c1ccccc1 ; COc1cnc2c(=O)n(-c3nc(-c4ccccc4)oc3-c3ccccc3)ccc2c1]

### 7-(2,5-diphenyloxazol-4-yl)-3-methoxy-1,7-naphthyridin-8(7H)-one. Compound 787

To a solution of 3-bromo-7-(2,5-diphenyloxazol-4-yl)-1,7-naphthyridin-8(7H)-one (120 mg, 1 Eq, 270 μmol) in MeOH (1 mL) and toluene (1 mL) were added cuprous bromide (77.5 mg, 2 Eq, 540 μmol) and sodium methanolate (0.23 g, 0.25 mL, 25 Wt %, 4 Eq, 1.08 mmol). The reaction was stirred at room temperature for 5 minutes and then refluxed for 3 hours. The reaction mixture was cooled, diluted with water, and stirred at room temperature for 1 hour. Acetic acid was added followed by chloroform/MeOH (10/1). The organic layer was separated, dried over MgSO₄, and concentrated in vacuo. The residue was purified by silica-gel column chromatography (10% MeOH in EtOAc) to give 7-(2,5-diphenyloxazol-4-yl)-3-methoxy-1,7-naphthyridin-8(7H)-one (21.5 mg, 54.4 μmol, 20.1%) as a pale-yellow solid.

¹H NMR: (400 MHz, DMSO-d₆) d ppm 4.00 (brs, 3H), 7.37-7.52 (m, 3H), 7.63 (brs, 8H), 8.17 (brs, 3H).

LCMS: rt 2.36 min. [M+H]⁺ 396.1 m/z.

### Synthesis of Compounds 788 & 789.

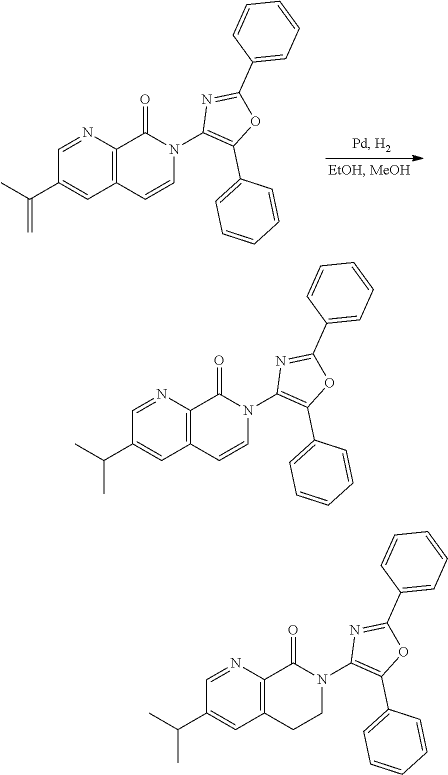 [SMILES: C=C(C)c1cnc2c(=O)n(-c3nc(-c4ccccc4)oc3-c3ccccc3)ccc2c1 ; CC(C)c1cnc2c(=O)n(-c3nc(-c4ccccc4)oc3-c3ccccc3)ccc2c1 ; CC(C)c1cnc2c(c1)CCN(c1nc(-c3ccccc3)oc1-c1ccccc1)C2=O]

### 7-(2,5-diphenyloxazol-4-yl)-3-isopropyl-1,7-naphthyridin-8(7H)-one and 7-(2,5-diphenyloxazol-4-yl)-3-isopropyl-6,7-dihydro-1,7-naphthyridin-8(5H)-one. Compounds 788 & 789

To a solution of 7-(2,5-diphenyloxazol-4-yl)-3-(prop-1-en-2-yl)-1,7-naphthyridin-8(7H)-one (40 mg, 1 Eq, 99 μmol) in EtOH (2 mL) and MeOH (1 mL) was added palladium on carbon (10 mol %, 8 mg, 0.8 Eq, 81 μmol) under nitrogen. While stirring at room temperature, hydrogen was introduced via balloon and the reaction was stirred for 5 hours. The reaction mixture was filtered, and the filtrate was concentrated in vacuo. The residue was purified by flash silica gel column chromatography (33% EtOAc in heptane) to give 7-(2,5-diphenyloxazol-4-yl)-3-isopropyl-1,7-naphthyridin-8(7H)-one (9.6 mg, 24 μmol, 24%) as a white solid and 7-(2,5-diphenyloxazol-4-yl)-3-isopropyl-6,7-dihydro-1,7-naphthyridin-8(5H)-one (2.0 mg, 4.9 μmol, 5.0%) as a white solid.

### 7-(2,5-diphenyloxazol-4-yl)-3-isopropyl-1,7-naphthyridin-8(7H)-one. Compound 788

¹H NMR: (400 MHz, CHLOROFORM-d) d ppm 1.42 (d, J=6.85 Hz, 6H), 1.49-1.58 (m, 1H), 6.53-6.61 (m, 1H), 7.33-7.42 (m, 3H), 7.53 (brs, 4H), 7.58-7.64 (m, 2H), 7.72-7.81 (m, 1H), 8.07-8.25 (m, 2H), 8.80-8.93 (m, 1H).

LCMS: rt 2.53 min. [M+H]⁺ 408.1 m/z.

### 7-(2,5-diphenyloxazol-4-yl)-3-isopropyl-6,7-dihydro-1,7-naphthyridin-8(5H)-one. Compound 789

¹H NMR: (400 MHz, CHLOROFORM-d) d ppm 1.37 (d, J=6.85 Hz, 6H), 2.72 (brs, 1H), 3.28 (d, J=4.89 Hz, 2H), 4.10 (brs, 2H), 7.35 (d, J=7.09 Hz, 1H), 7.38-7.46 (m, 2H), 7.47-7.55 (m, 4H), 7.74 (d, J=7.34 Hz, 2H), 8.08-8.15 (m, 2H), 8.62-8.71 (m, 1H).

LCMS: rt 2.38 min. [M+H]⁺ 410.1 m/z.

### Synthesis of Compound 790.

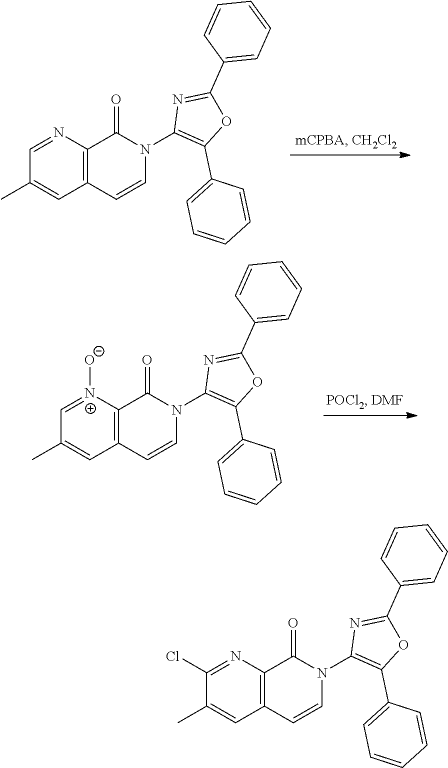

### 7-(2,5-diphenyloxazol-4-yl)-3-methyl-8-oxo-7,8-dihydro-1,7-naphthyridine 1-oxide

To a solution of 7-(2,5-diphenyloxazol-4-yl)-3-methyl-1,7-naphthyridin-8(7H)-one (128 mg, 1 Eq, 338 μmol) in DCM (2 mL) was added 3-chloroperoxybenzoic acid (0.13 g, 70 Wt %, 1.5 Eq, 507 μmol), and the reaction was stirred at room temperature for 18 hours. At this time, additional 3-chlorobenzoperoxoic acid (83 mg, 70 Wt %, 1 Eq, 338 μmol) was added, and the mixture was stirred at room temperature for 3 hours. The reaction mixture was concentrated. The residue was purified by silica gel flash column chromatography (19% MeOH in EtOAc) to give 7-(2,5-diphenyloxazol-4-yl)-3-methyl-8-oxo-7,8-dihydro-1,7-naphthyridine 1-oxide (23.7 mg, 59.9 μmol, 17.7%) as a pale-yellow solid.

¹H NMR: (400 MHz, CHLOROFORM-d) d ppm 2.59 (s, 3H), 6.50-6.60 (m, 1H), 7.38 (d, J=7.58 Hz, 4H), 7.51-7.66 (m, 5H), 7.72-7.77 (m, 1H), 8.13 (d, J=3.18 Hz, 3H).

LCMS: rt 2.19 min. [M+H]⁺ 396.0 m/z.

### 2-chloro-7-(2,5-diphenyloxazol-4-yl)-3-methyl-1,7-naphthyridin-8(7H)-one. Compound 790

To a solution of 7-(2,5-diphenyloxazol-4-yl)-3-methyl-8-oxo-7,8-dihydro-1,7-naphthyridine 1-oxide (23.7 mg, 1 Eq, 59.9 μmol) in DMF (1 mL) was added phosphorus oxychloride (13.8 mg, 8.35 μL, 1.5 Eq, 89.9 μmol) at 0° C. The reaction mixture was then stirred at room temperature for 3 hours. At this time, additional phosphoryl trichloride (13.8 mg, 8.35 μL, 1.5 Eq, 89.9 μmol) was added to the mixture and stirring was continued at room temperature for 18 hours. The reaction mixture was diluted with water. The precipitated solid was then collected by vacuum filtration. The solid was dissolved in DCM and concentrated in vacuo. The residue was washed with heptane: ethyl acetate (4:1), filtered, and dried under vacuum to give 2-chloro-7-(2,5-diphenyloxazol-4-yl)-3-methyl-1,7-naphthyridin-8(7H)-one (10.2 mg, 24.6 μmol, 41.1%) as a pale-yellow solid.

¹H NMR: (400 MHz, CHLOROFORM-d) d ppm 2.59 (s, 3H), 6.54 (d, J=7.58 Hz, 1H), 7.35 (m, 4H), 7.50-7.56 (m, 3H), 7.59 (d, J=6.85 Hz, 2H), 7.81 (s, 1H), 8.10-8.17 (m, 2H).

LCMS: rt 2.60 min. [M+H]⁺ 414.0 m/z.

### Synthesis of Compound 791.

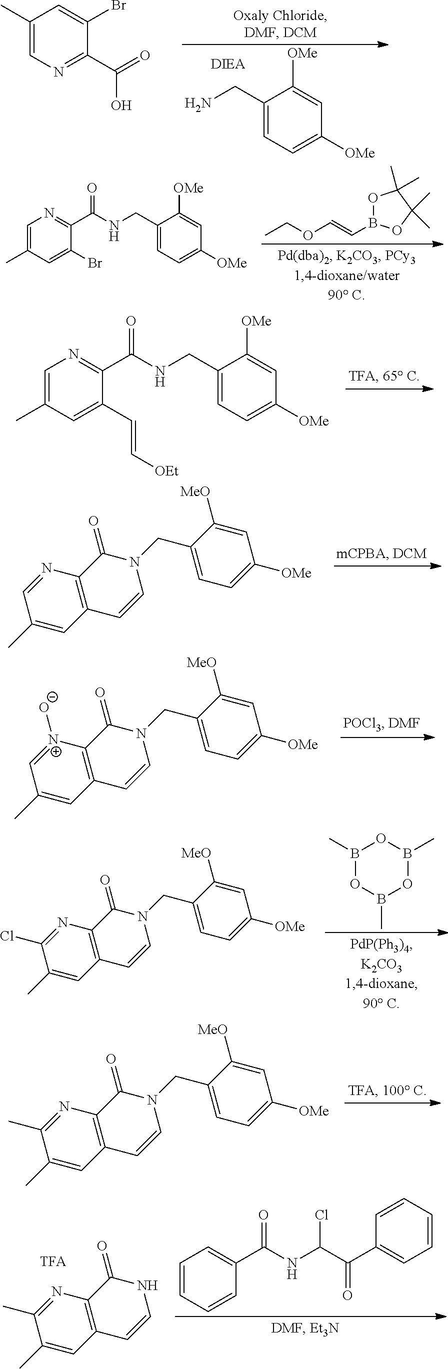

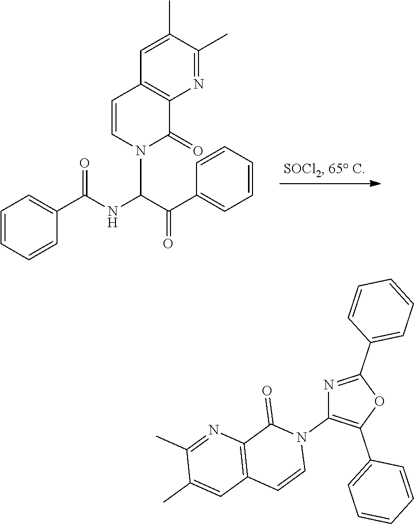 [SMILES: Cc1cc2ccn(C(NC(=O)c3ccccc3)C(=O)c3ccccc3)c(=O)c2nc1C ; Cc1cc2ccn(-c3nc(-c4ccccc4)oc3-c3ccccc3)c(=O)c2nc1C]

### 3-bromo-N-(2,4-dimethoxybenzyl)-5-methylpicolinamide

To a 0° C. slurry of 3-bromo-5-methylpicolinic acid (10.0 g, 1 Eq, 46.3 mmol) in DCM (150 mL) was added oxalyl dichloride (6.17 g, 4.17 mL, 1.05 Eq, 48.6 mmol) followed by N,N-dimethylformamide (169 mg, 0.18 mL, 0.05 Eq, 2.31 mmol). Gas evolved, the cold bath was removed, and the reaction was stirred to room temperature for 30 minutes. The reaction solution was chilled to 0° C. and treated with N-ethyl-N-isopropylpropan-2-amine (12.0 g, 16.1 mL, 2 Eq, 92.6 mmol) and (2,4-dimethoxyphenyl)methanamine (9.29 g, 8.35 mL, 1.2 Eq, 55.6 mmol). The resulting dark brown solution was stirred at room temperature for 14 hour. The reaction was washed with 0.5 N HCl (100 mL). The aqueous layer was extracted with 2×150 mL DCM. The combined organic layers were washed with brine, dried over MgSO₄, filtered, and concentrated to viscous dark brown oil. The crude product was purified by silica gel flash column chromatography (0-100% EtOAc in heptane) to give 3-bromo-N-(2,4-dimethoxybenzyl)-5-methylpicolinamide (9.5 g, 26 mmol, 56%) as brown solid.

LCMS: rt 2.18 min. [M+H]⁺ 365.0 m/z.

### (E)-N-(2,4-dimethoxybenzyl)-3-(2-ethoxyvinyl)-5-methylpicolinamide

To a solution of 3-bromo-N-(2,4-dimethoxybenzyl)-5-methylpicolinamide (8.1 g, 1 Eq, 22 mmol) in dioxane (80 mL) and water (8 mL) were added (E)-2-(2-ethoxyvinyl)-4,4,5,5-tetramethyl-1,3,2-dioxaborolane (4.5 g, 1.02 Eq, 23 mmol), potassium carbonate (9.2 g, 3.00 Eq, 67 mmol), tricyclohexylphosphine (0.62 g, 0.100 Eq, 2.2 mmol), and bis(dibenzylideneacetone)palladium (0.64 g, 0.05 Eq, 1.1 mmol). The reaction mixture was then stirred at 90° C. for 18 hours under nitrogen atmosphere. Water and EtOAc were added to the mixture, and the organic layer was separated. The organic layer was washed with brine, dried over MgSO₄, filtered, and concentrated in vacuo. The residue was purified by silica gel flash column chromatography (33% EtOAc in heptane) to give (E)-N-(2,4-dimethoxybenzyl)-3-(2-ethoxyvinyl)-5-methylpicolinamide (5.56 g, 15.6 mmol, 70%) as brown oil.

¹HNMR: (400 MHz, CHLOROFORM-d) d ppm 1.38 (t, J=6.72 Hz, 3H), 2.36 (s, 3H), 3.82 (s, 3H), 3.87 (s, 3H), 4.01 (q, J=6.85 Hz, 2H), 4.57 (d, J=5.62 Hz, 2H), 6.42-6.53 (m, 2H), 6.99 (d, J=3.20 Hz, 1H), 7.26 (brs, 1H), 7.57 (brs, 1H), 8.16 (s, 1H), 8.49 (brs, 1H).

LCMS: rt 2.21 min. [M+H]⁺ 357.1 m/z.

### 7-(2,4-dimethoxybenzyl)-3-methyl-1,7-naphthyridin-8(7H)-one

To (E)-N-(2,4-dimethoxybenzyl)-3-(2-ethoxyvinyl)-5-methylpicolinamide (6.75 g, 1 Eq, 18.9 mmol) was added TFA (59 g, 40 mL, 27 Eq, 0.52 mol), and the reaction mixture was heated to 65° C. for 2 hours. The reaction was cooled to room temperature and concentrated in vacuo. The residue was neutralized with saturated aqueous NaHCO₃ and extracted with DCM. The combined organic extractions were washed with brine, dried over MgSO₄, concentrated, and dried under vacuum to give 7-(2,4-dimethoxybenzyl)-3-methyl-1,7-naphthyridin-8(7H)-one (3.99 g, 12.9 mmol, 67.9%) as a pale-yellow solid.

¹H NMR: (400 MHz, CHLOROFORM-d) d ppm 3.61-4.00 (m, 6H), 6.29-6.57 (m, 5H), 7.33 (d, J=7.58 Hz, 1H), 7.44 (d, J=8.07 Hz, 1H), 7.70 (brs, 1H), 8.78 (brs, 1H).

LCMS: rt 1.78 min. [M-FH]+311.1 m/z.

### 7-(2,4-dimethoxybenzyl)-3-methyl-8-oxo-7,8-dihydro-1,7-naphthyridine 1-oxide

To a solution of 7-(2,4-dimethoxybenzyl)-3-methyl-1,7-naphthyridin-8(7H)-one (1.5 g, 1 Eq, 4.8 mmol) in DCM (15 mL) was added 3-chlorobenzoperoxoic acid (1.8 g, 70 Wt %, 1.5 Eq, 7.2 mmol), and the reaction mixture was stirred at room temperature for 2 hours. The reaction was concentrated, and the residue was purified by silica gel flash column chromatography (0-10% MeOH in DCM) to give 7-(2,4-dimethoxybenzyl)-3-methyl-8-oxo-7,8-dihydro-1,7-naphthyridine 1-oxide (1.48 g, 4.54 mmol, 94%) as a brown amorphous solid.

LCMS: rt 1.80 min. [M+H]⁺ 327.1 m/z.

### 2-chloro-7-(2,4-dimethoxybenzyl)-3-methyl-1,7-naphthyridin-8(7H)-one

To a solution of 7-(2,4-dimethoxybenzyl)-3-methyl-8-oxo-7,8-dihydro-1,7-naphthyridine 1-oxide (1.48 g, 1 Eq, 4.54 mmol) in DMF (10 mL) was added phosphorus oxychloride (1.39 g, 843 μL, 2 Eq, 9.07 mmol) at 0° C. The reaction mixture was stirred at room temperature for 18 hours. MeOH was poured into the crude reaction, and the mixture was concentrated. The residue was combined with water and extracted with DCM (2 times). The combined organic layers were dried over MgSO₄, filtered, and concentrated. The residue was purified by silica gel flash column chromatography (0-10% MeOH in DCM) to give 2-chloro-7-(2,4-dimethoxybenzyl)-3-methyl-1,7-naphthyridin-8(7H)-one (1.12 g, 3.25 mmol, 71.6%) as a brown oil.

LCMS: rt 2.19 min. [M+H]⁺ 345.1 m/z

### 7-(2,4-dimethoxybenzyl)-2,3-dimethyl-1,7-naphthyridin-8(7H)-one

To a solution of 2-chloro-7-(2,4-dimethoxybenzyl)-3-methyl-1,7-naphthyridin-8(7H)-one (319 mg, 1 Eq, 926 μmol) in dioxane (4 mL) were added 2,4,6-trimethyl-1,3,5,2,4,6-trioxatriborinane (233 mg, 529 μL, 3.5 molar, 2 Eq, 1.85 mmol), potassium carbonate (384 mg, 3 Eq, 2.78 mmol), and tetrakis(triphenylphosphine)palladium(0) (128 mg, 0.12 Eq, 111.1 μmol). The mixture was heated to 90° C. for 18 hours under nitrogen atmosphere. Upon cooling to room temperature, the reaction mixture was concentrated and the resulting residue was purified by silica gel flash column chromatography (100% EtOAc) to give 7-(2,4-dimethoxybenzyl)-2,3-dimethyl-1,7-naphthyridin-8(7H)-one (258 mg, 794 μmol, 85.8%) as a brown oil.

¹H NMR: (400 MHz, CHLOROFORM-d) ppm 2.42 (s, 3H), 2.73 (s, 3H), 3.77-3.90 (m, 6H), 5.32 (s, 2H), 6.41-6.52 (m, 1H), 7.43-7.53 (m, 3H), 7.54-7.61 (m, 1H), 7.64-7.74 (m, 1H).

LCMS: rt 2.16 min. [M+H]⁺ 325.1 m/z.

### 2,3-dimethyl-1,7-naphthyridin-8(7H)-one 2,2,2-trifluoroacetate

To 7-(2,4-dimethoxybenzyl)-2,3-dimethyl-1,7-naphthyridin-8(7H)-one (258 mg, 1 Eq, 794 μmol) was added TFA (3 g, 2 mL, 30 Eq, 0.03 mol), and the reaction mixture was heated to 100° C. for 2 hours. Upon cooling, the reaction mixture was concentrated and the resulting residue treated with MeOH and re-concentrated. The resulting material was washed with EtOAc/Heptane (1/5). The resulting solid was collected by vacuum filtration and dried in vacuo to give 2,3-dimethyl-1,7-naphthyridin-8(7H)-one 2,2,2-trifluoroacetate (196 mg, 80 Wt %, 543 μmol, 68%) as a brown solid.

LCMS: rt 0.23 min. [M+H]⁺ 175.1 m/z.

### N-(1-(2,3-dimethyl-8-oxo-1,7-naphthyridin-7(8H)-yl)-2-oxo-2-phenylethyl)benzamide

To a solution of N-(1-chloro-2-oxo-2-phenylethyl)benzamide (150 mg, 1 Eq, 548 μmol) in

DMF (2 mL) were added 2,3-dimethyl-1,7-naphthyridin-8(7H)-one 2,2,2-trifluoroacetate (196 mg, 80% Wt, 0.990 Eq, 543 μmol) and triethylamine (277 mg, 0.38 mL, 5 Eq, 2.74 mmol). The reaction was stirred at room temperature for 18 hours. Water was added to the reaction mixture and the resultant solid was collected by vacuum filtration. This material was washed with water and heptane and dried under vacuum to give N-(1-(2,3-dimethyl-8-oxo-1,7-naphthyridin-7(8H)-yl)-2-oxo-2-phenylethyl)benzamide (225 mg, 547 μmol, 99.8%) as a pale-yellow solid.

¹H NMR: (400 MHz, CHLOROFORM-d) d ppm 2.39 (s, 3H), 2.62 (s, 3H), 6.51 (d, J=7.34 Hz, 1H), 7.07 (d, J=7.34 Hz, 1H), 7.35-7.61 (m, 8H), 7.76 (d, J=7.34 Hz, 1H), 7.92 (t, J=7.21 Hz, 4H).

LCMS: rt 1.92 min. [M+H]⁺ 412.1 m/z.

### 7-(2,5-diphenyloxazol-4-yl)-2,3-dimethyl-1,7-naphthyridin-8(7H)-one. Compound 791

To N-(1-(2,3-dimethyl-8-oxo-1,7-naphthyridin-7(8H)-yl)-2-oxo-2-phenylethyl)benzamide (225 mg, 1 Eq, 547 μmol) in a 40 mL vial was added SOCl₂ (3 g, 2 mL, 50 Eq, 0.03 mol), and the reaction mixture was stirred at 65° C. for 2 hours. Upon cooling, the reaction was neutralized with saturated aqueous NaHCO₃ and extracted with DCM. The organic extracts were washed with brine, dried over MgSO₄ and concentrated. This resulting residue was purified by silica gel flash column chromatography (50% EtOAc in heptane) to give 7-(2,5-diphenyloxazol-4-yl)-2,3-dimethyl-1,7-naphthyridin-8(7H)-one as a pale-yellow solid.

¹H NMR: (400 MHz, CHLOROFORM-d) d ppm 2.51 (s, 3H), 2.80 (s, 3H), 6.45-6.64 (m, 1H), 7.37 (d, J=7.58 Hz, 4H), 7.48-7.56 (m, 3H), 7.59 (d, J=7.09 Hz, 2H), 7.72 (s, 1H), 8.14 (dd, J=6.60, 2.93 Hz, 2H).

LCMS: rt 2.23 min. [M+H]⁺ 394.1 m/z.

### Synthesis of Compound 792.

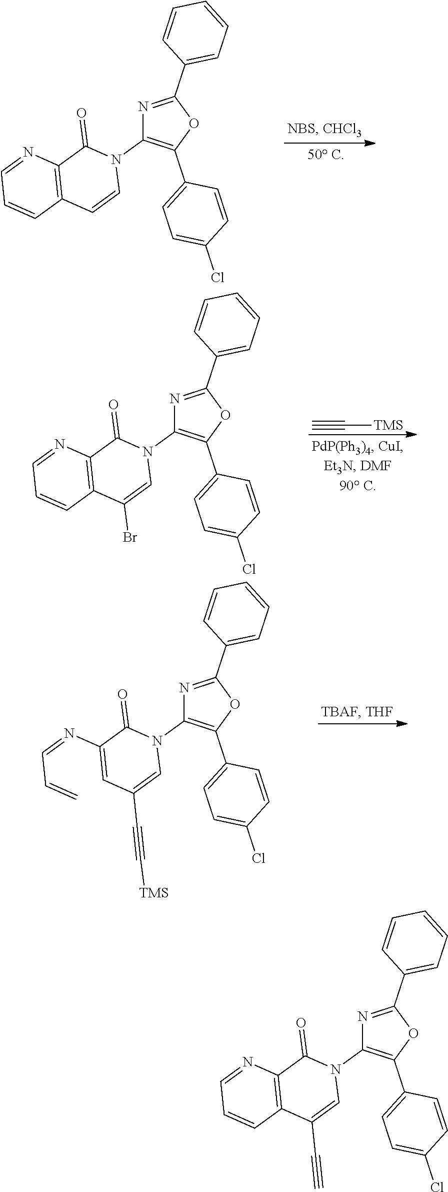

### 5-bromo-7-(5-(4-chlorophenyl)-2-phenyloxazol-4-yl)-1,7-naphthyridin-8(7H)-one

To a solution of 7-(5-(4-chlorophenyl)-2-phenyloxazol-4-yl)-1,7-naphthyridin-8(7H)-one (360 mg, 1 Eq, 900 μmol) in CHCl₃ (2 mL) was added NBS (192 mg, 1.2 Eq, 1.08 mmol), and the reaction mixture was heated to 50° C. for 1 hour. The reaction was cooled to room temperature, treated with 10% sodium thiosulfate (10 mL), and extracted with 2×35 mL of DCM. The combined organic layers were washed with brine, dried over MgSO₄, filtered, and concentrated to a dark brown paste. The crude product was purified by silica gel flash column chromatography (0-10% MeOH in DCM) to give 5-bromo-7-(5-(4-chlorophenyl)-2-phenyloxazol-4-yl)-1,7-naphthyridin-8(7H)-one (408 mg, 852 μmol, 94.7%) as brown solid.

LCMS: rt 3.10 min. [M+H]⁺ 477.6 m/z.

### 7-(5-(4-chlorophenyl)-2-phenyloxazol-4-3/1)-5-((trimethylsilyl)ethynyl)-1,7-naphthyridin-8(7H)-one

To a solution of 5-bromo-7-(5-(4-chlorophenyl)-2-phenyloxazol-4-yl)-1,7-naphthyridin-8(7H)-one (309 mg, 1 Eq, 645 μmol) in DMF (10 mL) were added ethynyltrimethylsilane (76.0 mg, 107 μL, 1.2 Eq, 774 μmol), cuprous iodide (12.3 mg, 0.1 Eq, 64.5 μmol), triethylamine (261 mg, 0.36 mL, 4 Eq, 2.58 mmol), and tetrakis(triphenylphosphine)palladium(0) (37.3 mg, 0.05 Eq, 32.3 μmol), and the reaction mixture was stirred at 50° C. for 5 hours. Upon cooling, the reaction was diluted in DCM and washed with 0.1 M aqueous EDTA and aqueous NH₄Cl. The organic layer was dried with MgSO₄ and dry-loaded onto silica. This material was purified by silica gel flash column chromatography (0-50% EtOAc in heptane) to give 7-(5-(4-chlorophenyl)-2-phenyloxazol-4-yl)-5-((trimethylsilyl)ethynyl)-1,7-naphthyridin-8(7H)-one (50 mg, 0.10 mmol, 16%) as a white film.

LCMS: rt 3.65 min. [M+H]⁺ 495.8 m/z.

### 7-(5-(4-chlorophenyl)-2-phenyloxazol-4-yl)-5-ethynyl-1,7-naphthyridin-8(7H)-one. Compound 792

7-(5-(4-Chlorophenyl)-2-phenyloxazol-4-yl)-5-((trimethylsilyl)ethynyl)-1,7-naphthyridin-8(7H)-one (206 mg, 415 μmol, 1 Eq) was dissolved in THF (1 mL) and TBAF (130 mg, 498 μL, 1.0 molar in THF, 1.2 Eq, 498 μmol) was added at room temperature, and the mixture was stirred for 15 minutes. The reaction was quenched with water and extracted with EtOAc. The organic layer was concentrated and redissolved in 1 mL of DMF. This solution was purified by reverse phase prep-HPLC (C18 column, 10-100% MeCN in water with 0.1% TFA) to give 7-(5-(4-chlorophenyl)-2-phenyloxazol-4-yl)-5-ethynyl-1,7-naphthyridin-8(7H)-one (12 mg, 28 μmol, 6.8%) as a beige solid.

¹H NMR: (DMSO-d₆, 400 MHz) δ=9.03-9.01 (dd, J=4.4 Hz, 1.6 Hz, 1H), 8.44-8.41 (dd, J=8.4 Hz, 1.6 Hz, 1H), 8.26 (s, 1H), 8.24-8.21 (m, 2H), 8.03-8.00 (q, 4.4 Hz, 1H), 7.70-7.68 (m, 5H), 7.62-7.60 (m, 2H), 4.64 (s, 1H).

LCMS: rt 2.95 min. [M+H]⁺ 423.8 m/z

### Synthesis of Compound 793.

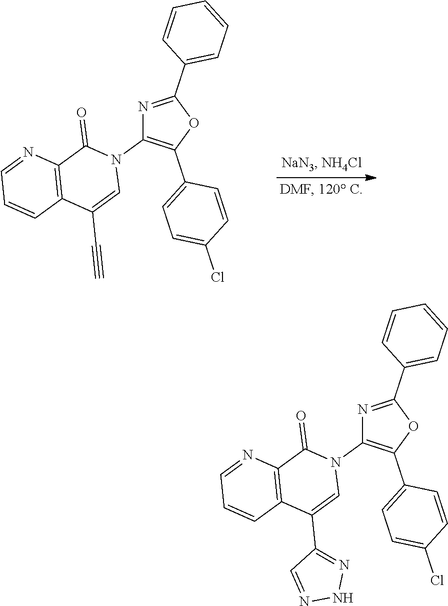 [SMILES: C#Cc1cn(-c2nc(-c3ccccc3)oc2-c2ccc(Cl)cc2)c(=O)c2ncccc12 ; O=c1c2ncccc2c(-c2cn[nH]n2)cn1-c1nc(-c2ccccc2)oc1-c1ccc(Cl)cc1]

### 7-(5-(4-chlorophenyl)-2-phenyloxazol-4-yl)-5-(2H-1,2,3-triazol-4-yl)-1,7-naphthyridin-8(7H)-one. Compound 793

7-(5-(4-Chlorophenyl)-2-phenyloxazol-4-yl)-5-ethynyl-1,7-naphthyridin-8(7H)-one (100 mg, 1 Eq, 236 μmol), ammonium chloride (37.9 mg, 3 Eq, 708 μmol), and sodium azide (23.0 mg, 1.5 Eq, 354 μmol) were combined in DMF (0.5 mL), and the reaction mixture was stirred at 120° C. for 16 hours. The reaction was quenched with water and extracted with DCM. The organic solution was concentrated, and the resulting crude material was purified by silica gel column chromatography (0-10% MeOH in DCM) to yield 7-(5-(4-chlorophenyl)-2-phenyloxazol-4-yl)-5-(2H-1,2,3-triazol-4-yl)-1,7-naphthyridin-8(7H)-one (7 mg, 0.01 mmol, 6%).

1H NMR: (DMSO-d₆, 400 MHz) δ=8.97-8.96 (dd, J=4.4 Hz, 1.6 Hz, 1H), 8.87-8.85 (d, J=8.4 Hz, 1H), 8.20-8.17 (m, 2H), 7.93-7.99 (q, J=4.4 Hz, 1H), 7.88-7.86 (m, 1H), 7.67 (s, 1H), 7.65-7.63 (m, 4H), 7.58-7.56 (m, 2H), 7.54-7.51 (m, 1H), 7.47-7.43 (t, J=7.6 Hz, 1H).

LCMS: rt 2.51 min. [M+H]⁺ 466.9 m/z.

### Example Procedures for Compounds Containing Various Trisubstituted 5-Membered Heterocycles

### Synthesis of Compound 794.

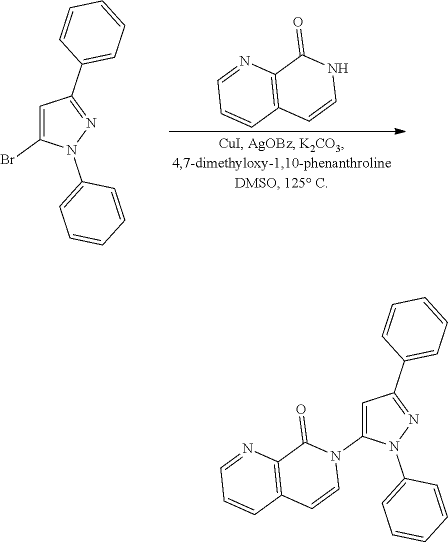 [SMILES: Brc1cc(-c2ccccc2)nn1-c1ccccc1 ; O=c1[nH]ccc2cccnc12 ; O=c1c2ncccc2ccn1-c1cc(-c2ccccc2)nn1-c1ccccc1]

### 7-(1,3-diphenyl-1H-pyrazol-5-yl)-1,7-naphthyridin-8(7H)-one. Compound 794

To a 40 mL vial were added 5-bromo-1,3-diphenyl-1H-pyrazole (ref. Journal of Heterocyclic Chemistry (2012), 49(1), 183, 100 mg, 1 Eq, 334 μmol), copper(I) iodide (6.37 mg, 0.1 Eq, 33.4 μmol), silver(I) benzoate (76.5 mg, 1 Eq, 334 μmol), 1,7-naphthyridin-8(7H)-one (58.6 mg, 1.2 Eq, 401 μmol), potassium carbonate (92.4 mg, 2 Eq, 669 μmol), 4,7-dimethoxy-1,10-phenanthroline (12.0 mg, 0.15 Eq, 50.1 μmol), and DMSO (6.7 mL). The reaction was stirred at 125° C. for 72 hours. Upon cooling, the reaction was diluted with water and extracted with EtOAc 3 times. The combined organic extracts were then washed with brine, dried over MgSO₄, and concentrated in vacuo. The crude material was purified by a silica gel column chromatography (0-100% EtOAc in heptane) to afford 7-(1,3-diphenyl-1H-pyrazol-5-yl)-1,7-naphthyridin-8(7H)-one (12 mg, 33 μmol, 9.9%) as a white solid.

¹H NMR (400 MHz, DMSO-d₆) d ppm 6.73-6.79 (m, 1H), 7.31-7.64 (m, 8H), 7.67-7.73 (m, 1H), 7.74-7.82 (m, 1H), 7.92-8.01 (m, 2H), 8.17-8.26 (m, 1H), 8.81-8.88 (m, 1H), 9.00-9.05 (m, 1H).

LCMS: rt 2.17 min. [M+H]⁺ 365.1 m/z.

### Synthesis of Compound 795.

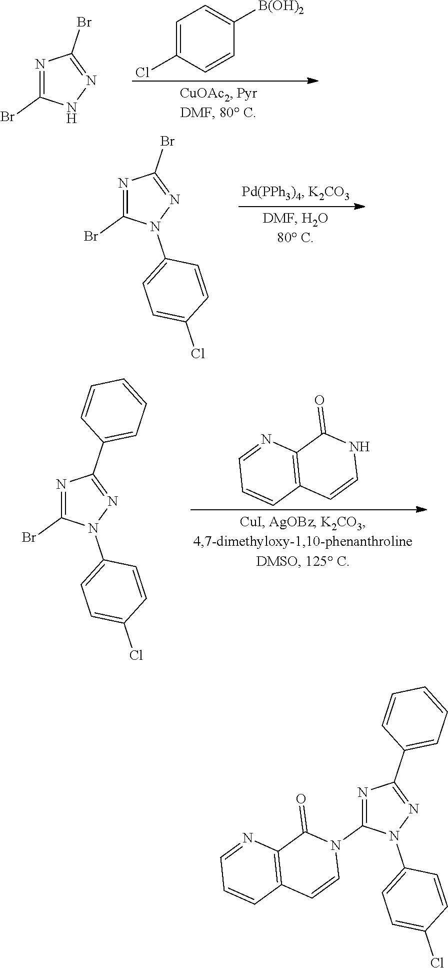 [SMILES: Brc1n[nH]c(Br)n1 ; OB(O)c1ccc(Cl)cc1 ; Clc1ccc(-n2nc(Br)nc2Br)cc1 ; Clc1ccc(-n2nc(-c3ccccc3)nc2Br)cc1 ; O=c1[nH]ccc2cccnc12 ; O=c1c2ncccc2ccn1-c1nc(-c2ccccc2)nn1-c1ccc(Cl)cc1]

### 3,5-dibromo-1-(4-chlorophenyl)-1H-1,2,4-triazole

(4-Chlorophenyl)boronic acid (1.4 g, 1 Eq, 8.8 mmol), 3,5-dibromo-1H-1,2,4-triazole (2.0 g, 1 Eq, 8.8 mmol), copper (II) acetate (3.4 g, 2.1 Eq, 19 mmol), and pyridine (2.1 g, 2.1 mL, 3 Eq, 26 mmol) were combined in a flame dried vial. A scoop (500 mg) of preactivated 4 Å molecular sieves was added. The vial was then evacuated and backfilled with 02. With the oxygen balloon fixed, the reagents were dissolved in anhydrous DMF (25.0 mL) and stirred at 80° C. for 12 hours. Upon cooling, the reaction was quenched with 1 M aqueous HCl, and the resulting precipitate was collected by vacuum filtration and washed with water. The precipitate was dissolved in DCM, concentrated, and purified by silica gel column chromatography (0-100% DCM in heptane) to afford 3,5-dibromo-1-(4-chlorophenyl)-1H-1,2,4-triazole (1.2 g, 3.6 mmol, 40%) as a white solid.

LCMS: rt 2.42 min. [M+H]⁺ 335.9 m/z.

### 5-bromo-1-(4-chlorophenyl)-3-phenyl-1H-1,2,4-triazole

3,5-Dibromo-1-(4-chlorophenyl)-1H-1,2,4-triazole (250 mg, 1 Eq, 741 μmol), tetrakis(triphenylphosphine)palladium(0) (85.6 mg, 0.1 Eq, 74.1 μmol), phenylboronic acid (99.4 mg, 1.1 Eq, 815 μmol), and potassium carbonate (205 mg, 2 Eq, 1.48 mmol) were combined in a flame dried vial. A scoop (500 mg) of preactivated 4 Å molecular sieves was added. The vial was then evacuated and backfilled with N₂. With the N₂ balloon fixed, the reagents were dissolved in anhydrous DMF (3.0 mL) and water (0.60 mL) and stirred at 80° C. for 12 hours. At this time, the crude mixture was diluted with water and extracted with EtOAc (3 times). The combined organic extracts were washed with brine, dried with MgSO₄, and concentrated. The crude mixture was then purified by reverse phase preparative HPLC (C18 column, 10-100% MeCN in water with 0.1% TFA) to afford 5-bromo-1-(4-chlorophenyl)-3-phenyl-1H-1,2,4-triazole (167 mg, 499 μmol, 67.4%) as a light solid.

LCMS: rt 2.59 min. [M+H]⁺ 333.9 m/z.

### 7-(1-(4-chlorophenyl)-3-phenyl-1H-1,2,4-triazol-5-yl)-1,7-naphthyridin-8(7H)-one. Compound 795

7-(1-(4-chlorophenyl)-3-phenyl-1H-1,2,4-triazol-5-yl)-1,7-naphthyridin-8(7H)-one was prepared in a similar fashion to 7-(1,3-diphenyl-1H-pyrazol-5-yl)-1,7-naphthyridin-8(7H)-one, compound 508. ¹H NMR (400 MHz, DMSO-d₆) δ ppm 6.77-6.83 (m, 1H), 7.45-7.60 (m, 7H), 7.62-7.76 (m, 1H), 7.78-7.84 (m, 1H), 8.21-8.28 (m, 1H), 8.85-8.91 (m, 1H).

LCMS: rt 2.18 min. [M+H]⁺ 401.

### Synthesis of Compound 796.

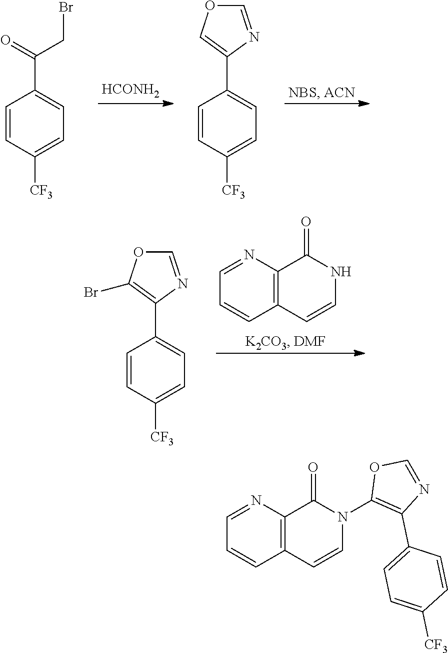

### 4-[4-(trifluoromethyl)phenyl]-1,3-oxazole

4-(Trifluoromethyl)phenacyl bromide (17.0 g, 63.7 mmol, 1.0 eq) and formamide (38.1 mL, 955 mmol, 15.0 eq) were combined and heated at 130° C. for 1 hour. The reaction was cooled to room temperature and diluted with ethyl acetate and water. The aqueous layer was then extracted with ethyl acetate 3 times. The combined organic layers were dried over sodium sulfate, filtered, and concentrated in vacuo. The crude material was purified by normal phase chromatography (silica gel, 0-5% ethyl acetate in hexanes) to give 4-[4-(trifluoromethyl)phenyl]-1,3-oxazole (4.63 g, 21.7 mmol, 34%).

1H NMR (300 MHz, DMSO-d6) δ 8.83 (d, J=1.0 Hz, 1H), 8.55 (d, J=1.0 Hz, 1H), 8.02 (d, J=8.1 Hz, 2H), 7.81 (d, J=8.2 Hz, 2H).

### 5-bromo-4-[4-(trifluoromethyl)phenyl]-1,3-oxazole

4-[4-(Trifluoromethyl)phenyl]-1,3-oxazole (3.45 g, 16.2 mmol, 1.0 eq) and N-bromosuccinimide (3.47 g, 19.5 mmol, 1.2 eq) were dissolved in acetonitrile (71.4 mL) and mixture was stirred at 50° C. for 2 hours. The reaction was cooled to room temperature and diluted with ethyl acetate and water. The layers were separated, and the aqueous layer was extracted with ethyl acetate 3 times. The combined organic layers were dried over sodium sulfate, filtered, and concentrated in vacuo. The crude material was purified by normal phase column chromatography (silica gel, 0-1% ethyl acetate in hexanes) to give 5-bromo-4-[4-(trifluoromethyl)phenyl]-1,3-oxazole (1.8 g, 6.25 mmol, 38%).

UPLC-MS: RT=2.65 min, [M+H]+=293.00, 96%.

### 7-{4-[4-(trifluoromethyl)phenyl]-1,3-oxazol-5-yl}-7,8-dihydro-1,7-naphthyridin-8-one

5-Bromo-4-[4-(trifluoromethyl)phenyl]-1,3-oxazole (0.48 g, 1.64 mmol, 1.0 eq), 7,8-dihydro-1,7-naphthyridin-8-one (0.24 g, 1.64 mmol, 1.0 eq), and potassium carbonate (0.454 g, 3.29 mmol, 2.0 eq) were dissolved in anhydrous DMF (10.0 mL), and the reaction mixture was stirred at 100° C. for 3 hours. The reaction was cooled to room temperature and diluted with ethyl acetate and brine. The layers were separated, and the aqueous layer was extracted with ethyl acetate (3 times). The combined organic layers were dried over sodium sulfate and concentrated. The crude material was purified by silica gel column chromatography (0-3% MeOH in DCM) to afford 7-{4-[4-(trifluoromethyl)phenyl]-1,3-oxazol-5-yl}-7,8-dihydro-1,7-naphthyridin-8-one (0.2 g, 0.504 mmol, 31%).

¹H NMR: (300 MHz, DMSO-d6) δ 8.90 (dd, J=4.4, 1.6 Hz, 1H), 8.73 (s, 1H), 8.28 (dd, J=8.2, 1.7 Hz, 1H), 7.89-7.71 (m, 5H), 7.66 (d, J=7.4 Hz, 1H), 6.89 (d, J=7.5 Hz, 1H).

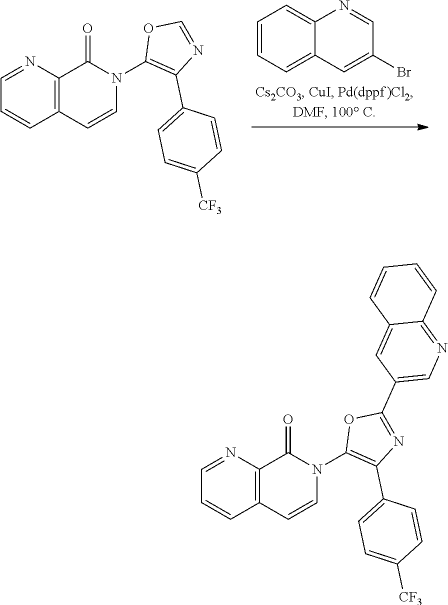 [SMILES: O=c1c2ncccc2ccn1-c1ocnc1-c1ccc(C(F)(F)F)cc1 ; Brc1cnc2ccccc2c1 ; O=c1c2ncccc2ccn1-c1oc(-c2cnc3ccccc3c2)nc1-c1ccc(C(F)(F)F)cc1]

### 7-(2-(quinolin-3-yl)-4-(4-(trifluoromethyl)phenyl)oxazol-5-yl)-1,7-naphthyridin-8(7H)-one. Compound 796

Under inert atmosphere, 7-{4-[4-(trifluoromethyl)phenyl]-1,3-oxazol-5-yl}-7,8-dihydro-1,7-naphthyridin-8-one (24 mg, 0.0673 mmol, 1 eq), 3-bromoquinoline (0.021 g, 0.101 mmol, 1.5 eq), and cesium carbonate (0.066 g, 0.202 mmol, 3.0 eq) were dissolved in anhydrous DMF (0.6 mL). The resulting mixture was purged with argon for 5 min. Copper(I) iodide (0.001 g, 0.007 mmol, 0.1 eq) and 1,1′-bis(diphenylphosphino)ferrocene dichloropalladium(II) (0.005 g, 0.007 mmol, 0.1 eq) were added. The reaction was stirred for 48 hours at 100° C. Upon cooling, the solvent was removed under reduced pressure. The residue was purified by preparative HPLC (C18 column, 10-100% MeCN in water with 0.1% TFA) to give 7-(2-(quinolin-3-yl)-4-(4-(trifluoromethyl)phenyl)oxazol-5-yl)-1,7-naphthyridin-8(7H)-one (0.002 g, 0.004 mmol, 6%).

m/z+1=485.15

¹H NMR: (400 MHz, DMSO-d6) δ 9.59 (d, J=2.2 Hz, 1H), 9.16 (d, J=2.3 Hz, 1H), 8.93 (dd, J=4.4, 1.7 Hz, 1H), 8.31 (dd, J=8.2, 1.7 Hz, 1H), 8.28-8.23 (m, 1H), 8.16 (d, J=8.4 Hz, 1H), 7.89 (tt, J=8.8, 7.4 Hz, 6H), 7.81-7.73 (m, 2H), 6.96 (d, J=7.4 Hz, 1H).

### Synthesis of Compound 797.

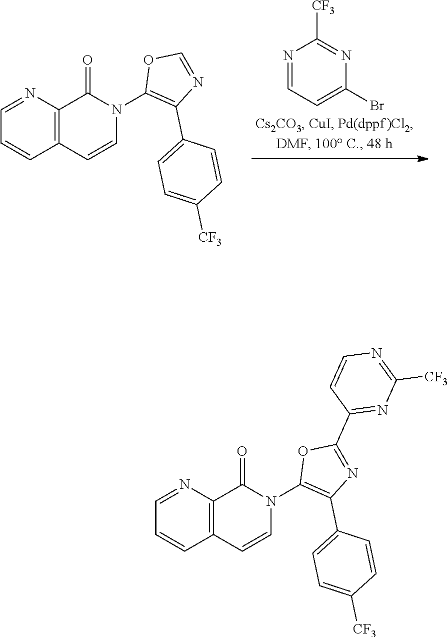 [SMILES: O=c1c2ncccc2ccn1-c1ocnc1-c1ccc(C(F)(F)F)cc1 ; FC(F)(F)c1nccc(Br)n1 ; O=c1c2ncccc2ccn1-c1oc(-c2ccnc(C(F)(F)F)n2)nc1-c1ccc(C(F)(F)F)cc1]

### 7-{4-[4-(trifluoromethyl)phenyl]-2-[2-(trifluoromethyl)pyrimidin-4-yl]-1,3-oxazol-5-yl}-7,8-dihydro-1,7-naphthyridin-8-one. Compound 797

Under inert atmosphere, 7-{4-[4-(trifluoromethyl)phenyl]-1,3-oxazol-5-yl}-7,8-dihydro-1,7-naphthyridin-8-one) (0.024 g, 0.067 mmol, 1.0 eq), 4-bromo-2-(trifluoromethyl)pyrimidine (0.023 g, 0.101 mmol, 1.5 eq), and cesium carbonate (0.066 g, 0.202 mmol, 3.0 eq) were dissolved in anhydrous DMF (0.6 mL). The resulting mixture was purged with argon for 5 minutes. Copper(I) iodide (0.001 g, 0.007 mmol, 0.1 eq) and 1,1′-bis(diphenylphosphino)ferrocene dichloropalladium(II) (0.005 g, 0.007 mmol, 0.1 eq) were added. The reaction mixture was stirred for 48 hours at 100° C. Upon cooling, the solvent was removed under reduced pressure. The residue was redissolved in ethyl acetate, washed with brine, dried over sodium sulfate, and concentrated under reduced pressure. The crude product was purified by preparative HPLC (C18 column, 10-100% MeCN in water with 0.1% TFA) to give 7-{4-[4-(trifluoromethyl)phenyl]-2-[2-(trifluoromethyl)pyrimidin-4-yl]-1,3-oxazol-5-yl}-7,8-dihydro-1,7-naphthyridin-8-one (0.003 g, 0.006 mmol, 9%).

m/z+1=504.13

¹H NMR: (400 MHz, DMSO-d6) δ 9.36 (d, J=5.3 Hz, 1H), 8.92 (dd, J=4.4, 1.6 Hz, 1H), 8.59 (d, J=5.2 Hz, 1H), 8.30 (dd, J=8.1, 1.7 Hz, 1H), 7.93-7.82 (m, 5H), 7.74 (d, J=7.4 Hz, 1H), 6.96 (d, J=7.5 Hz, 1H).

### Synthesis of Compound 798.

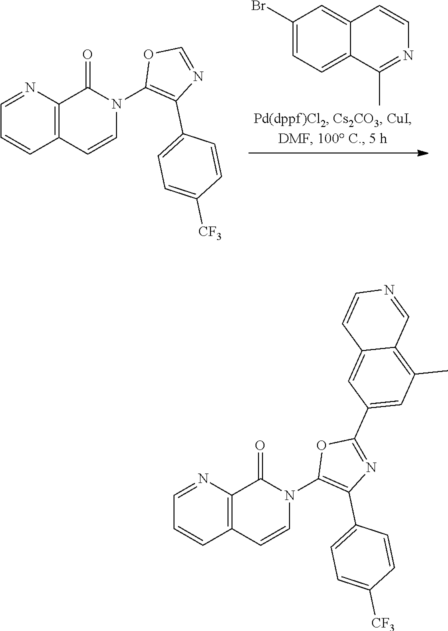 [SMILES: O=c1c2ncccc2ccn1-c1ocnc1-c1ccc(C(F)(F)F)cc1 ; Cc1nccc2cc(Br)ccc12 ; Cc1cc(-c2nc(-c3ccc(C(F)(F)F)cc3)c(-n3ccc4cccnc4c3=O)o2)cc2ccncc12]

### 7-[2-(1-methylisoquinolin-6-yl)-4-[4-(trifluoromethyl)phenyl]-1,3-oxazol-5-yl]-7,8-dihydro-1,7-naphthyridin-8-one. Compound 798

Under inert atmosphere, 7-{4-[4-(trifluoromethyl)phenyl]-1,3-oxazol-5-yl}-7,8-dihydro-1,7-naphthyridin-8-one) (0.036 g, 0.091 mmol, 1.0 eq), 6-bromo-1-methylisoquinoline (0.034 g, 0.136 mmol, 1.5 eq), and cesium carbonate (0.089 g, 0.272 mmol, 3.0 eq) were placed in a reaction flask and anhydrous DMF (0.72 mL) was added. The resulting mixture was bubbled with argon for 20 minutes, and copper(I) iodide (0.002 g, 0.009 mmol, 0.1 eq) and 1,1′-bis(diphenylphosphino)ferrocene dichloropalladium(II) (0.007 g, 0.009 mmol, 0.1 eq) were added. The reaction was stirred for 5 h at 100° C. The solvent was evaporated under reduced pressure. The residue was dissolved in dichloromethane, brine was added, and the layers were separated. The aqueous layer was extracted with dichloromethane (3 times). The combined organic layers were dried over sodium sulfate and concentrated. The crude solid was purified preparative HPLC (C18 column, 10-100% MeCN in water with 0.1% TFA) to give 7-[2-(1-methylisoquinolin-6-yl)-4-[4-(trifluoromethyl)phenyl]-1,3-oxazol-5-yl]-7,8-dihydro-1,7-naphthyridin-8-one (0.021 g, 0.041 mmol, 46%).

m/z+1=499.13

¹H NMR: (300 MHz, DMSO-d6) δ 8.93 (dd, J=4.5, 1.6 Hz, 1H), 8.76 (d, J=1.7 Hz, 1H), 8.49-8.43 (m, 2H), 8.33 (ddd, J=14.7, 8.4, 1.7 Hz, 2H), 7.97-7.73 (m, 7H), 6.95 (d, J=7.4 Hz, 1H), 2.97 (s, 3H).

### Synthesis of Compound 799.

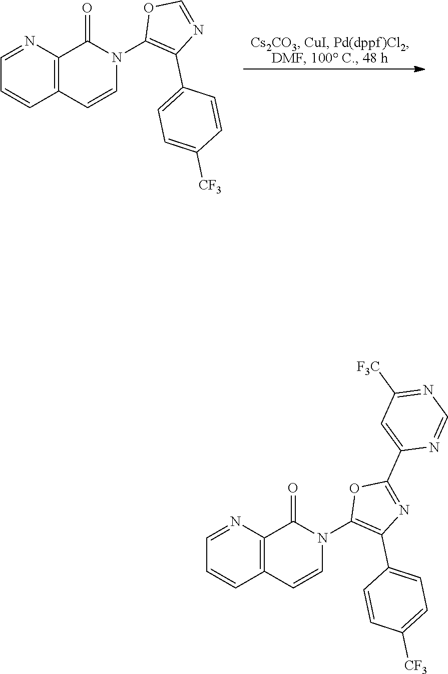 [SMILES: O=c1c2ncccc2ccn1-c1ocnc1-c1ccc(C(F)(F)F)cc1 ; "Cs2CO3, CuI, Pd(dppf)Cl2,DMF, 100° C., 48 h" ; O=c1c2ncccc2ccn1-c1oc(-c2cc(C(F)(F)F)ncn2)nc1-c1ccc(C(F)(F)F)cc1]

### 7-{4-[4-(trifluoromethyl)phenyl]-2-[6-(trifluoromethyl)pyrimidin-4-yl]-1,3-oxazol-5-yl}-7,8-dihydro-1,7-naphthyridin-8-one. Compound 799

7-{4-[4-(Trifluoromethyl)phenyl]-1,3-oxazol-5-yl}-7,8-dihydro-1,7-naphthyridin-8-one (0.05 g, 0.14 mmol, 1.0 eq), 4-bromo-6-(trifluoromethyl)pyrimidine (0.032 g, 0.14 mmol, 1.0 eq), and cesium carbonate (0.137 g, 0.42 mmol, 3.0 eq) were placed in a reaction flask and anhydrous DMF (1.0 mL) was added. The resulting mixture was bubbled with argon for 20 minutes, and copper(I) iodide (0.003 g, 0.014 mmol, 0.1 eq) and 1,1′-bis(diphenylphosphino)ferrocene dichloropalladium(II) (0.01 g, 0.014 mmol, 0.1 eq) were added. The reaction was stirred for 48 hours at 100° C. The crude mixture was concentrated under reduced pressure. The resulting material was directly purified by preparative HPLC (C18 column, 10-100% MeCN in water with 0.1% TFA) to give 7-{4-[4-(trifluoromethyl)phenyl]-2-[6-(trifluoromethyl)pyrimidin-4-yl]-1,3-oxazol-5-yl}-7,8-dihydro-1,7-naphthyridin-8-one (0.003 g, 0.006 mmol, 4%).

m/z+1=504.08

¹H NMR: (400 MHz, DMSO-d6) δ 9.67 (d, J=1.3 Hz, 1H), 8.92 (s, 1H), 8.66 (d, J=1.4 Hz, 1H), 8.32-8.28 (m, 1H), 7.88 (q, J=8.4 Hz, 5H), 7.75 (d, J=7.4 Hz, 1H), 6.96 (d, J=7.4 Hz, 1H).

### Synthesis of Compound 800.

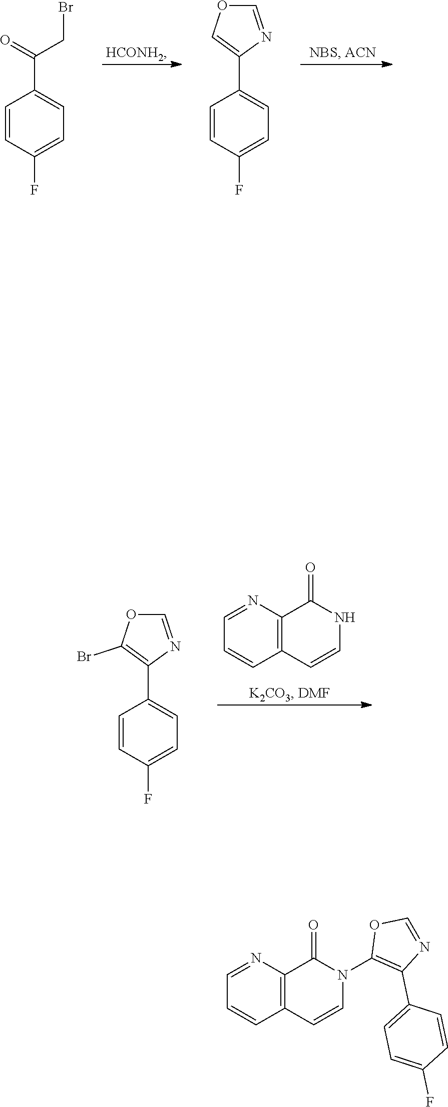

### 4-(4-fluorophenyl)-1,3-oxazole

2-Bromo-4′-fluoroacetophenone (22.8 g, 105 mmol, 1.0 eq) and formamide (62.8 mL, 1.58 mol, 15.0 eq) were combined and heated at 130° C. for 3 hours. The reaction was cooled to room temperature and diluted with ethyl acetate and water. The layers were separated, and the aqueous layer was extracted with ethyl acetate 3 times. The combined organic layers were dried over sodium sulfate, filtered, and concentrated in vacuo. The crude material was purified by normal phase chromatography (silica gel, 0-5% ethyl acetate in hexanes) to give 4-(4-fluorophenyl)-1,3-oxazole (5.8 g, 35.6 mmol, 34%) as a solid.

1H NMR (300 MHz, DMSO-d6) δ 8.62 (d, J=0.8 Hz, 1H), 8.46 (d, J=1.0 Hz, 1H), 7.86-7.81 (m, 2H), 7.31-7.25 (m, 2H).

### 5-bromo-4-(4-fluorophenyl)-1,3-oxazole

4-(4-Fluorophenyl)-1,3-oxazole (5.8 g, 35.6 mmol, 1.0 eq) and N-bromosuccinimide (7.6 g, 42.7 mmol, 1.2 eq) were dissolved in acetonitrile (140 mL), and the reaction mixture was stirred at 50° C. for 2 hours. At this time, a second portion of N-bromosuccinimide (0.3 eq) was added and the reaction mixture was stirred at 50° C. for 2 hours. The reaction mixture was cooled to room temperature and diluted with ethyl acetate and water. The layers were separated, and the aqueous layer was extracted with ethyl acetate 3 times. The combined organic layers were dried over sodium sulfate, filtered, and concentrated in vacuo. The crude material was purified using normal phase chromatography (silica gel, 0-1% ethyl acetate in hexanes) to give 5-bromo-4-(4-fluorophenyl)-1,3-oxazole (5.3 g, 21.9 mmol, 61%).

UPLC-MS: RT=3.68 min, [M+H]+=241.75, 100%.

1H NMR (600 MHz, DMSO-d6) δ 8.64 (s, 1H), 7.98-7.92 (m, 2H), 7.39-7.32 (m, 2H).

### 7-[4-(4-fluorophenyl)-1,3-oxazol-5-yl]-7,8-dihydro-1,7-naphthyridin-8-one

To a stirred suspension of 7,8-dihydro-1,7-naphthyridin-8-one (1.81 g, 12.4 mmol, 1.0 eq) in DMF (60.0 mL) was added sodium hydride (0.595 g, 24.8 mmol, 2.0 eq) and the resulting mixture was stirred for 20 minutes at room temperature. 5-Bromo-4-(4-fluorophenyl)-1,3-oxazole (3.0 g, 12.4 mmol, 1.0 eq) was then added and the mixture was stirred at 120° C. for 5 hours. The reaction was cooled to room temperature and diluted with ethyl acetate. Water was added and extraction with ethyl acetate was performed (3 times). The combined organic layers were dried over sodium sulfate and concentrated. The residue was triturated with diethyl ether to give 7-[4-(4-fluorophenyl)-1,3-oxazol-5-yl]-7,8-dihydro-1,7-naphthyridin-8-one (1.4 g, 2.78 mmol, 22%) as an orange solid.

1H NMR: (300 MHz, DMSO-d6) δ 8.88 (dd, J=4.4, 1.6 Hz, 1H), 8.64 (s, 1H), 8.25 (dd, J=8.1, 1.6 Hz, 1H), 7.83 (dd, J=8.1, 4.4 Hz, 1H), 7.61 (d, J=7.4 Hz, 1H), 7.55 (dd, J=5.4, 2.3 Hz, 1H), 7.31-7.19 (m, 3H), 6.86 (d, J=7.4 Hz, 1H).

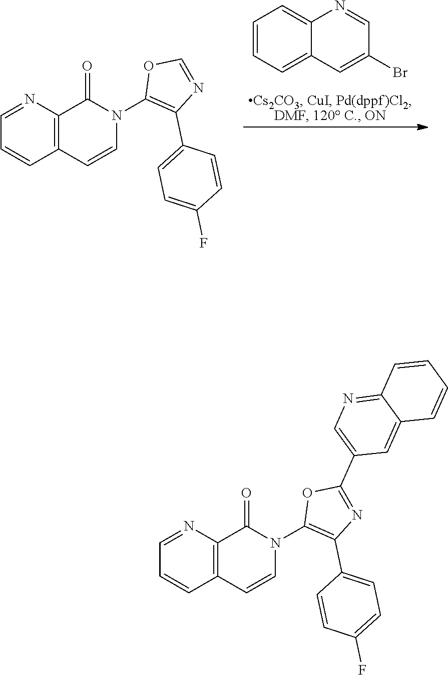

### 7-[4-(4-fluorophenyl)-2-(quinolin-3-yl)-1,3-oxazol-5-yl]-7,8-dihydro-1,7-naphthyridin-8-one. Compound 800

Under inert atmosphere, 7-[4-(4-fluorophenyl)-1,3-oxazol-5-yl]-7,8-dihydro-1,7-naphthyridin-8-one (0.3 g, 0.596 mmol, 1.0 eq), 3-bromoquinoline (0.186 g, 0.893 mmol, 1.5 eq), and cesium carbonate (0.582 g, 1.79 mmol, 3.0 eq) were placed in a reaction tube and anhydrous DMF (6.0 mL) was added. Next, the suspension was bubbled with argon for 10 minutes. Copper(I) iodide (0.011 g, 0.06 mmol, 0.1 eq) and 1,1′-bis(diphenylphosphino)ferrocene dichloropalladium(II) (0.044 g, 0.06 mmol, 0.1 eq) were added. The reaction tube was sealed, and the mixture was stirred at 120° C. for 16 hours. Upon cooling, the reaction was filtered through a celite pad, and the celite pad was washed with methanol. The combined solvents were removed under reduced pressure. The crude material was purified by silica gel column chromatography (0-5% MeOH in DCM). The combined product containing fractions were concentrated and triturated with a small amount of methanol and diethyl ether. This material was collected by vacuum filtration and dried under vacuum to give 7-[4-(4-fluorophenyl)-2-(quinolin-3-yl)-1,3-oxazol-5-yl]-7,8-dihydro-1,7-naphthyridin-8-one (0.095 g, 0.216 mmol, 36%) as a beige solid.

m/z+1=435.00

¹H NMR: (300 MHz, DMSO-d6) δ 9.57 (d, J=2.2 Hz, 1H), 9.12 (d, J=2.0 Hz, 1H), 8.92 (dd, J=4.4, 1.6 Hz, 1H), 8.31-8.21 (m, 2H), 8.14 (d, J=8.3 Hz, 1H), 7.94-7.84 (m, 2H), 7.78-7.69 (m, 4H), 7.36-7.29 (m, 2H), 6.92 (d, J=7.5 Hz, 1H).

### Synthesis of Compound 801.

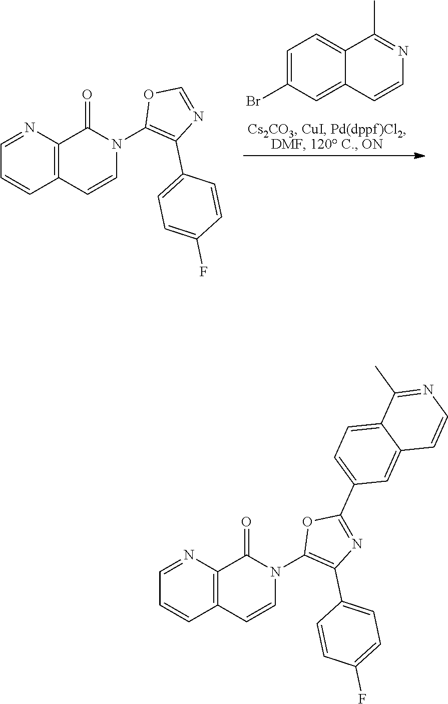

### 7-[4-(4-fluorophenyl)-2-(1-methylisoquinolin-6-yl)-1,3-oxazol-5-yl]-7,8-dihydro-1,7-naphthyridin-8-one. Compound 801

Under inert atmosphere, 7-[4-(4-fluorophenyl)-1,3-oxazol-5-yl]-7,8-dihydro-1,7-naphthyridin-8-one (0.2 g, 0.397 mmol, 1.0 eq), 6-bromo-1-methylisoquinoline (0.106 g, 0.476 mmol, 1.2 eq), and cesium carbonate (0.388 g, 1.19 mmol, 3.0 eq) were placed in a reaction tube and anhydrous DMF (4.0 mL) was added. The suspension was bubbled with argon for 10 minutes. Then, copper(I) iodide (0.008 g, 0.04 mmol, 0.1 eq) and 1,1′-bis(diphenylphosphino)ferrocene dichloropalladium(II) (0.029 g, 0.04 mmol, 0.1 eq) were added. The reaction tube was sealed, and the reaction mixture was stirred at 120° C. for 16 hours. The crude reaction was filtered through a celite pad, and the celite pad was washed with methanol. The combined solvents were removed under reduced pressure. The crude material was purified by silica gel column chromatography (0-5% MeOH in DCM). The product containing fractions were combined and concentrated. The resulting material was triturated with a small amount of methanol and diethyl ether. This material was the collected by vacuum filtration and dried under vacuum to give 7-[4-(4-fluorophenyl)-2-(1-methylisoquinolin-6-yl)-1,3-oxazol-5-yl]-7,8-dihydro-1,7-naphthyridin-8-one (0.015 g, 0.033 mmol, 8%) as a beige solid.

m/z−1=449.00.

¹H NMR: (300 MHz, DMSO-d6) δ 8.91 (dd, J=4.4, 1.6 Hz, 1H), 8.72 (d, J=1.5 Hz, 1H), 8.47-8.40 (m, 2H), 8.31 (ddd, J=12.9, 8.5, 1.7 Hz, 2H), 7.92-7.84 (m, 2H), 7.77 (d, J=7.4 Hz, 1H), 7.74-7.66 (m, 2H), 7.36-7.28 (m, 2H), 6.92 (d, J=7.5 Hz, 1H), 2.95 (s, 3H).

### Synthesis of Compound 802.

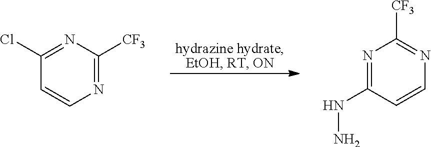 [SMILES: FC(F)(F)c1nccc(Cl)n1 ; "hydrazine hydrate,EtOH, RT, ON" ; NNc1ccnc(C(F)(F)F)n1]

4-Hydrazinyl-2-(trifluoromethyl)pyrimidine. To a solution of 4-chloro-2-(trifluoromethyl)pyrimidine (0.3 g, 1.64 mmol, 1.0 eq) in ethanol (1.5 mL) was added hydrazine monohydrate (0.395 g, 3.95 mmol, 2.4 eq), and the reaction mixture was stirred at room temperature for 16 hours. Brine was added, and the reaction mixture was extracted with dichloromethane 3 times. The combined organic layers were dried over sodium sulfate and concentrated to give 4-hydrazinyl-2-(trifluoromethyl)pyrimidine (0.24 g, 1.35 mmol, 82%).

¹H NMR: (300 MHz, DMSO-d6) δ 9.07 (s, 1H), 8.28 (s, 1H), 6.98 (s, 1H), 4.57 (s, 2H).

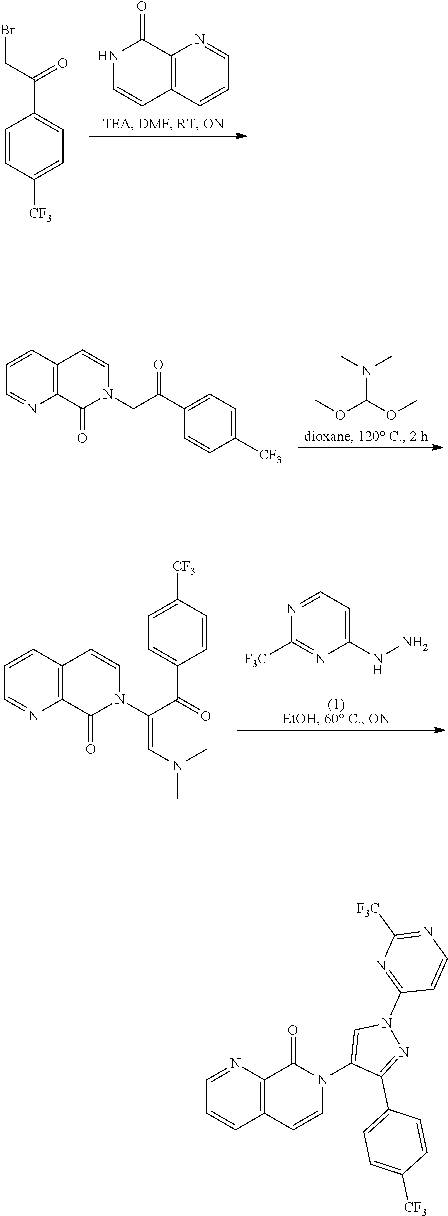

### 7-{2-oxo-2-[4-(trifluoromethyl)phenyl]ethyl}-7,8-dihydro-1,7-naphthyridin-8-one

7,8-Dihydro-1,7-naphthyridin-8-one (2.74 g, 18.7 mmol, 1.0 eq) was dissolved in DMF (60.0 mL) and triethylamine (13.0 mL, 93.6 mmol, 5.0 eq) was added. The reaction mixture was stirred for 10 minutes at room temperature. Next, 4-(trifluoromethyl)phenacyl bromide (5.0 g, 18.7 mmol, 1.0 eq) was added, and the reaction mixture was stirred at for 2 hours at temperature. Brine was added, and the reaction mixture was extracted with ethyl acetate 3 times. The combined organic layers were dried over sodium sulfate and concentrated to give a crude product. The crude material was purified by silica gel column chromatography (0-30% EtOAc in hexanes) to give 7-{2-oxo-2-[4-(trifluoromethyl)phenyl]ethyl}-7,8-dihydro-1,7-naphthyridin-8-one (2.3 g, 6.78 mmol, 36%).

UPLC-MS: RT=1.94 min, M+1=333.0, 98%.

### 7-[(1Z)-1-(dimethylamino)-3-oxo-3-[4-(trifluoromethyl)phenyl]prop-1-en-2-yl]-7,8-dihydro-1,7-naphthyridin-8-one

7-{2-Oxo-2-[4-(trifluoromethyl)phenyl]ethyl}-7,8-dihydro-1,7-naphthyridin-8-one (1.12 g, 3.30 mmol, 1.0 eq) and N,N-dimethylformamide dimethyl acetal (1.32 mL, 9.91 mmol, 3.0 eq) were dissolved in dioxane (8.96 mL), and the reaction mixture was stirred at 120° C. for 4 hours. At this time, the reaction was cooled to room temperature and stirred for 16 hours. The reaction mixture was concentrated, and the obtained crude material was triturated with ethyl acetate, filtered, and dried to give 7-[(1Z)-1-(dimethylamino)-3-oxo-3-[4-(trifluoromethyl)phenyl]prop-1-en-2-yl]-7,8-dihydro-1,7-naphthyridin-8-one (0.9 g, 2.14 mmol, 65%).

UPLC-MS: RT=1.76 min, M+1=388.0, 92%.

### 7-(3-(4-(trifluoromethyl)phenyl)-1-(2-(trifluoromethyl)pyrimidin-4-yl)-1H-pyrazol-4-yl)-1,7-naphthyridin-8(7H)-one. Compound 802

7-[(1Z)-1-(Dimethylamino)-3-oxo-3-[4-(trifluoromethyl)phenyl]prop-1-en-2-yl]-7,8-dihydro-1,7-naphthyridin-8-one (0.2 g, 0.434 mmol, 1.0 eq) and 4-hydrazinyl-2-(trifluoromethyl)pyrimidine (0.155 g, 0.867 mmol, 2.0 eq) were dissolved in ethanol (4.0 mL), and the reaction mixture was stirred for 16 hours at 70° C. Upon cooling to room temperature, the reaction mixture was concentrated. The crude material was purified via preparative TLC (3% MeOH in DCM, eluted twice) to give 7-(3-(4-(trifluoromethyl)phenyl)-1-(2-(trifluoromethyl)pyrimidin-4-yl)-1H-pyrazol-4-yl)-1,7-naphthyridin-8(7H)-one (0.009 g, 0.018 mmol, 4%).

m/z+1=503.13

¹H NMR: (400 MHz, DMSO-d6) δ 9.16 (d, J=5.7 Hz, 1H), 8.80 (dd, J=4.4, 1.7 Hz, 1H), 8.43 (s, 1H), 8.33 (d, J=5.7 Hz, 1H), 8.13 (dd, J=8.2, 1.7 Hz, 1H), 7.79-7.68 (m, 3H), 7.62 (d, J=8.1 Hz, 2H), 7.49 (d, J=7.4 Hz, 1H), 6.62 (d, J=7.5 Hz, 1H).

### Synthesis of Compound 803.

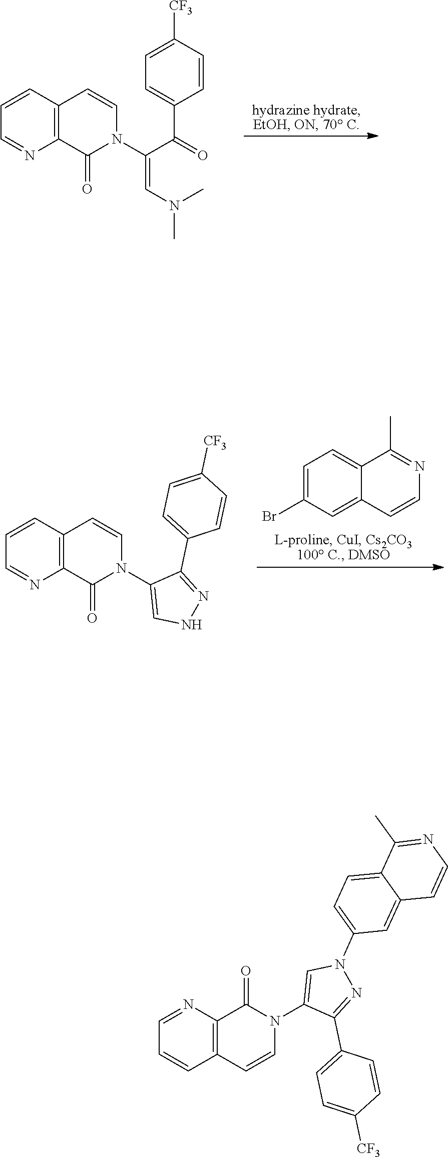

### 7-{3-[4-(trifluoromethyl)phenyl]-1H-pyrazol-4-yl}-7,8-dihydro-1,7-naphthyridin-8-one

7-[(1Z)-1-(Dimethylamino)-3-oxo-3-[4-(trifluoromethyl)phenyl]prop-1-en-2-yl]-7,8-dihydro-1,7-naphthyridin-8-one (0.2 g, 0.475 mmol, 1.0 eq) and hydrazine monohydrate (0.038 g, 1.19 mmol, 2.5 eq) were dissolved in ethanol (4.0 mL), and the reaction mixture was stirred for 16 hours at 70° C. Upon cooling, the reaction mixture was concentrated. The obtained crude material was triturated with methanol, collected by vacuum filtration, and dried in vacuo to give 7-{3-[4-(trifluoromethyl)phenyl]-1H-pyrazol-4-yl}-7,8-dihydro-1,7-naphthyridin-8-one (0.107 g, 0.3 mmol, 63%).

UPLC-MS: RT=1.86 min, M+1=356.95, 96% @ 254 nm

¹H NMR: (300 MHz, DMSO-d6) δ 13.53 (s, 1H), 8.82 (dd, J=4.4, 1.6 Hz, 1H), 8.27-8.10 (m, 2H), 7.79-7.68 (m, 3H), 7.63 (d, J=8.1 Hz, 2H), 7.47 (d, J=7.3 Hz, 1H), 6.70 (d, J=7.4 Hz, 1H).

### 7-[1-(1-methylisoquinolin-6-yl)-3-[4-(trifluoromethyl)phenyl]-1H-pyrazol-4-yl]-7,8-dihydro-1,7-naphthyridin-8-one. Compound 803

7-{3-[4-(Trifluoromethyl)phenyl]-1H-pyrazol-4-yl}-7,8-dihydro-1,7-naphthyridin-8-one (0.072 g, 0.202 mmol, 1.0 eq), 6-bromo-1-methylisoquinoline (0.075 g, 0.303 mmol, 1.5 eq), and cesium carbonate (0.132 g, 0.404 mmol, 2.0 eq) were suspended in anhydrous DMSO (0.72 mL). The reaction mixture was purged with argon for around 10 minutes. Then, L-proline (0.005 g, 0.04 mmol, 0.2 eq) and copper(I) iodide (0.004 g, 0.02 mmol, 0.1 eq) were added. The reaction mixture was stirred at 100° C. for 18 hours. Brine was added, and the reaction mixture was extracted with ethyl acetate 3 times. The combined organic layers were dried over sodium sulfate and concentrated to give a crude material. The crude material was purified via preparative TLC (2% MeOH in DCM, eluted twice) to give 7-[1-(1-methylisoquinolin-6-yl)-3-[4-(trifluoromethyl)phenyl]-1H-pyrazol-4-yl]-7,8-dihydro-1,7-naphthyridin-8-one (0.024 g, 0.047 mmol, 23%).

m/z=498.19+ [M+H]+

¹H NMR: (300 MHz, Methanol-d4) δ 9.03 (s, 1H), 8.87 (d, J=4.5 Hz, 1H), 8.47 (d, J=9.0 Hz, 2H), 8.42-8.32 (m, 2H), 8.25 (d, J=8.1 Hz, 1H), 7.82 (dd, J=10.6, 6.7 Hz, 4H), 7.68 (d, J=8.2 Hz, 2H), 7.55 (d, J=7.4 Hz, 1H), 6.84 (d, J=7.4 Hz, 1H), 3.01 (s, 3H).

### Synthesis of Compound 804.

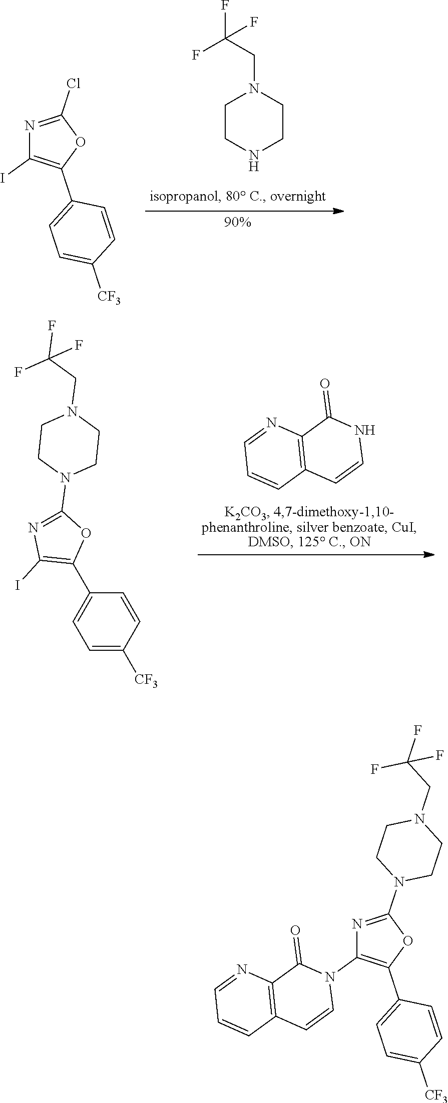

1-{4-iodo-5-[4-(trifluoromethyl)phenyl]-1,3-oxazol-2-yl}-4-(2,2,2-trifluoroethyl)piperazine. 2-Chloro-4-iodo-5-[4-(trifluoromethyl)phenyl]-1,3-oxazole (0.2 g, 0.535 mmol, 1.0 eq.), 1-(2,2,2-trifluoroethyl)piperazine·2HCl (0.129 g, 0.535 mmol, 0.999 eq.), and N,N-diisopropylethylamine (0.28 mL, 1.61 mmol, 3.00 eq.) were dissolved in isopropanol (8.0 mL). The reaction was stirred at 80° C. for 16 hours. Upon cooling, the crude reaction was concentrated under reduced pressure. Diethyl ether was added to the crude material, and a white precipitate formed. The precipitate was filtered off, and the filtrate was evaporated to obtain 1-{4-iodo-5-[4-(trifluoromethyl)phenyl]-1,3-oxazol-2-yl}-4-(2,2,2-trifluoroethyl)piperazine (0.26 g, 0.515 mmol, 88%).

UPLC-MS: RT=4.60 min, [M+H]⁺=505.90, 92% @254 nm.

### 7-{2-[4-(2,2,2-trifluoroethyl)-1-piperazinyl]-5-[p-(trifluoromethyl)phenyl]-1,3-oxazol-4-yl}-1,7-diaza-8(7H)-naphthalenone. Compound 804

1-{4-iodo-5-[4-(trifluoromethyl)phenyl]-1,3-oxazol-2-yl}-4-(2,2,2-trifluoroethyl)piperazine and 1,7-naphthyridin-8(7H)-one were combined and exposed to similar conditions as those which produced compound 508.

UPLC-MS: [M+H]⁺=524.2

Additional Compounds were Prepared in a Manner Analogous to Compound 804.

| | Exact mass | Observed | |
|---|---|---|---|
| Compound | (g/mol) | m/z(+) | Assignment |
| 804 | 523.1443 | 524.2 | [M + H]+ |
| 805 | 474.1304 | 475.1 | [M + H]+ |
| 806 | 475.1256 | 476.0 | [M + H]+ |
| 807 | 494.1566 | 495.1 | [M + H]+ |
| 808 | 497.1675 | 497.9 | [M + H]+ |
| 809 | 504.1409 | 505.1 | [M + H]+ |
| 810 | 504.1409 | 505.1 | [M + H]+ |
| 811 | 522.1315 | 523.1 | [M + H]+ |

### Synthesis of Compound 812

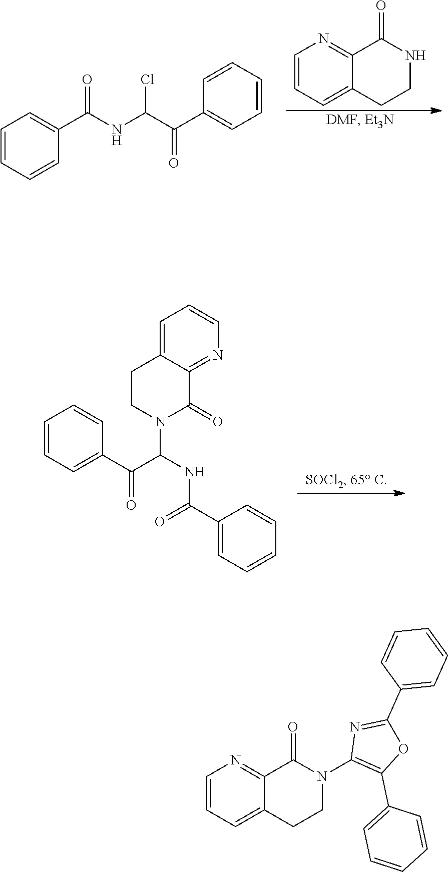

### N-(2-oxo-1-(8-oxo-5,8-dihydro-1,7-naphthyridin-7(6H)-yl)-2-phenylethyl)benzamide

To a solution of 6,7-dihydro-1,7-naphthyridin-8(5H)-one (97.4 mg, 1.2 Eq, 658 μmol) in DMF (5.5 mL) was added N-(1-chloro-2-oxo-2-phenylethyl)benzamide (150 mg, 1 Eq, 548 μmol). The reaction turned cloudy orange, and it was stirred at room temperature for 1 hour. At this time, water was added, and the mixture was extracted with ethyl acetate. The combined organics were washed with brine, dried over MgSO₄, filtered, and concentrated. The residue was purified by flash silica gel column chromatography (10% MeOH in EtOAc) and dried under vacuum to give N-(2-oxo-1-(8-oxo-5,8-dihydro-1,7-naphthyridin-7(6H)-yl)-2-phenylethyl)benzamide (16.9 mg, 43.8 μmol, 8.0%) as a white solid. ¹H NMR: (400 MHz, CHLOROFORM-d) δ ppm 2.99-3.13 (m, 2H), 3.86-4.07 (m, 2H), 6.86-6.96 (m, 1H), 7.31-7.38 (m, 1H), 7.43-7.66 (m, 6H), 7.93 (d, J=7.34 Hz, 2H), 8.09 (d, J=7.34 Hz, 2H), 8.24-8.32 (m, 1H), 8.58-8.73 (m, 1H).

LCMS: rt 1.87 min. [M+H]⁺ 386.1 m/z.

### 7-(2,5-diphenyloxazol-4-yl)-6,7-dihydro-1,7-naphthyridin-8(5H)-one. Compound 812

To N-(2-oxo-1-(8-oxo-5,8-dihydro-1,7-naphthyridin-7(6H)-yl)-2-phenylethyl)benzamide (50.0 mg, 1 Eq, 130 μmol) in a 40 mL vial, was added SOCl₂ (309 mg, 189 μL, 20 Eq, 2.59 mmol), and the reaction mixture was stirred at 65° C. for 4 hours. The reaction mixture was concentrated to remove excess thionyl chloride. The residue was re-dissolved in DCM, neutralized slowly with by saturated NaHCO₃, and the layers separated. The aqueous layer was extracted with DCM. The combined organic layers were washed with brine, dried over MgSO₄, filtered, and concentrated in vacuo. The residue was triturated with diethyl ether and dried in vacuo to give 7-(2,5-diphenyloxazol-4-yl)-6,7-dihydro-1,7-naphthyridin-8(5H)-one (9.5 mg, 26 μmol, 20%) as a white solid.

¹H NMR: (400 MHz, CHLOROFORM-d) δ ppm 3.22-3.41 (m, 2H), 4.05-4.20 (m, 2H), 7.32-7.37 (m, 1H), 7.40-7.45 (m, 2H), 7.51 (d, J=2.45 Hz, 4H), 7.68-7.82 (m, 3H), 8.05-8.17 (m, 2H), 8.76-8.90 (m, 1H).

LCMS: rt 2.24 min. [M+H]⁺ 368.0 m/z.

### Synthesis of Compound 813

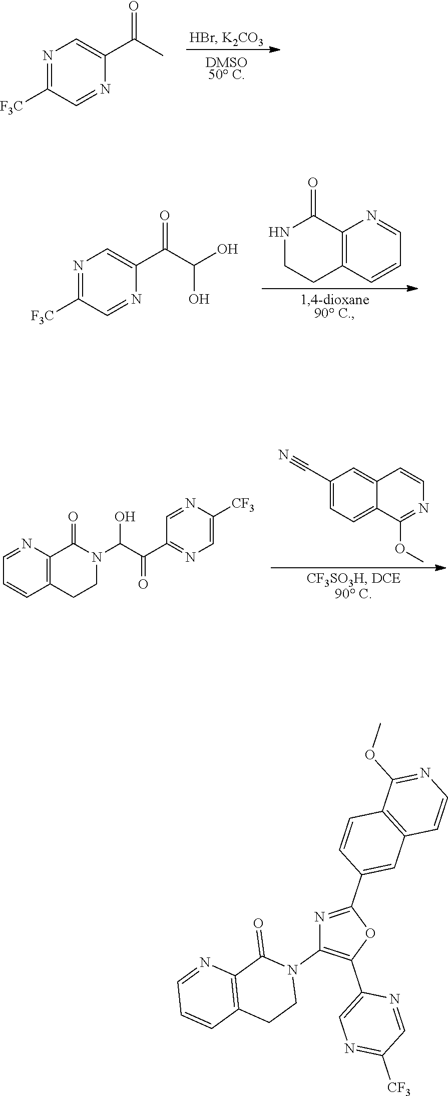

### 2,2-dihydroxy-1-(5-(trifluoromethyl)pyrazin-2-yl)ethan-1-one

To a stirred solution of 1-(5-(trifluoromethyl)pyrazin-2-yl)ethan-1-one (2.50 g, 1 Eq, 13.1 mmol) in DMSO was added aqueous HBr (6.65 g, 4.46 mL, 48 Wt %, 3 Eq, 39.4 mmol) dropwise, and the reaction was heated to 50° C. for 16 hours. The reaction was cooled to room temperature, diluted with water (50 mL), basified with potassium carbonate (5.45 g, 3 Eq, 39.4 mmol), and extracted with EtOAc (2×75 mL). The combined organic layers were washed with brine, dried over Na₂SO₄, filtered, and concentrated under vacuum to afford 2,2-dihydroxy-1-(5-(trifluoromethyl)pyrazin-2-yl)ethan-1-one (817 mg, 3.67 mmol, 28%) as yellow solid.

¹H NMR (400 MHz, D₂O): δ ppm 8.99-9.03 (m, 2H), 5.07-5.15 (m, 1H).

### 7-(1-hydroxy-2-oxo-2-(5-(trifluoromethyl)pyrazin-2-yl)ethyl)-6,7-dihydro-1,7-naphthyridin-8(5H)-one

2,2-Dihydroxy-1-(5-(trifluoromethyl)pyrazin-2-yl)ethan-1-one (124 mg, 1.1 Eq, 557 μmol) and 6,7-dihydro-1,7-naphthyridin-8(5H)-one (75.0 mg, 1 Eq, 506 μmol) were combined in 1,4-dioxane (5 mL). The reaction mixture was heated to 90° C. for 4 hours. At this time, the reaction mixture was cooled to room temperature and concentrated. The resulting residue was purified by flash silica gel column chromatography (20% MeOH in DCM) to give 7-(1-hydroxy-2-oxo-2-(5-(trifluoromethyl)pyrazin-2-yl)ethyl)-6,7-dihydro-1,7-naphthyridin-8(5H)-one (150 mg, 426 lima 84%) as white foam.

LCMS: rt 1.69 min. [M+H]⁺ 353.0 m/z.

### 7-(2-(1-methoxyisoquinolin-6-yl)-5-(5-(trifluoromethyl)pyrazin-2-yl)oxazol-4-yl)-6,7-dihydro-1,7-naphthyridin-8(5H)-one. Compound 813

To a slurry of 7-(1-hydroxy-2-oxo-2-(5-(trifluoromethyl)pyrazin-2-yl)ethyl)-6,7-dihydro-1,7-naphthyridin-8(5H)-one (22 mg, 1 Eq, 62 μmol) and 1-methoxyisoquinoline-6-carbonitrile (13 mg, 1.1 Eq, 69 μmol) in DCE (2 mL) was added trifluoromethanesulfonic acid (47 mg, 28 μL, 5 Eq, 0.31 mmol). The resulting cloudy reaction mixture was stirred at 90° C. for 1 hour. Upon cooling, the reaction was concentrated to a sticky brown solid. The residue was dissolved in DCM (30 mL) and washed with saturated aqueous NaHCO₃. The aqueous layer was extracted with DCM (30 mL). The combined organics were washed with brine, dried over MgSO₄, filtered, and concentrated to a yellow solid. The crude material was triturated with hot MeOH and the resulting precipitation was collected by vacuum filtration, washed with MeOH, and dried under vacuum to give 7-(2-(1-methoxyisoquinolin-6-yl)-5-(5-(trifluoromethyl)pyrazin-2-yl)oxazol-4-yl)-6,7-dihydro-1,7-naphthyridin-8(5H)-one (14 mg, 27 μmol, 43%) as white solid.

¹H NMR (400 MHz, DMSO) δ 9.24 (d, J=8.8 Hz, 2H), 8.80 (s, 1H), 8.68 (d, J=4.6 Hz, 1H), 8.44-8.32 (m, 2H), 8.16 (d, J=5.9 Hz, 1H), 7.94 (d, J=7.8 Hz, 1H), 7.69-7.58 (m, 2H), 4.26 (t, J=6.4 Hz, 2H), 4.12 (s, 3H), 3.35 (d, J=6.6 Hz, 2H).

LCMS: rt 2.73 min. [M+H]⁺ 519.2 m/z.

### Synthesis of Compound 814

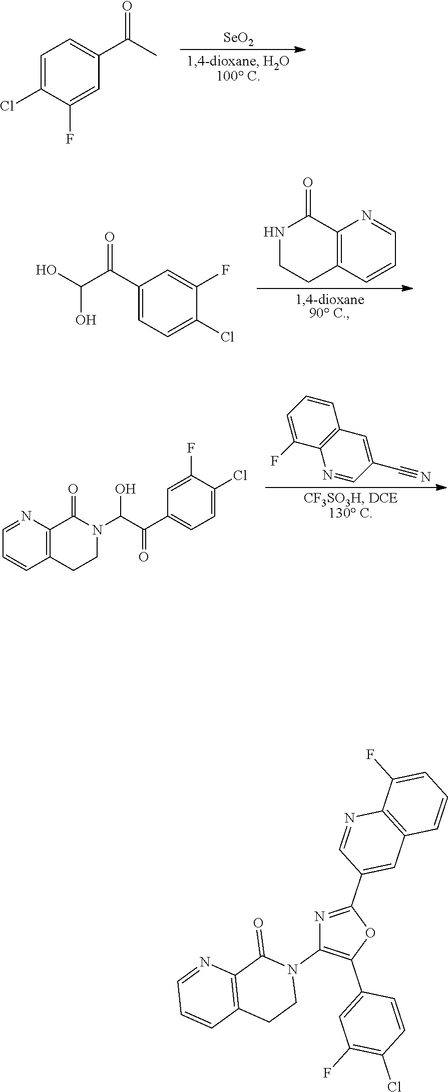

### 1-(4-chloro-3-fluorophenyl)-2,2-dihydroxyethan-1-one

Selenium dioxide (25.4 g, 95 Wt %, 1.5 Eq, 217 mmol) was placed in water (30.0 mL) and 1,4-dioxane (300 mL) in a 500 mL medium pressure flak and heated to 70° C. until all material completely dissolved. To the reaction mixture at 70° C., was added 1-(4-chloro-3-fluorophenyl)ethan-1-one (25.0 g, 1 Eq, 145 mmol), and the mixture was stirred at 100° C. for 21 hours. The reaction was cooled to room temperature, filtered through a pad of celite, and washed with EtOAc. The liquid filtrate was concentrated to a viscous brown oil which was treated with water (50 mL) and heated to 100° C. under a reflux condenser for 8 hours. It was then cooled and stirred at room temperature for 16 hours. The resulting precipitate was collected by vacuum filtration, washed with cold water, and dried under vacuo to give 2-(4-chloro-3-fluorophenyl)-2-oxoacetaldehyde (24.1 g, 129 mmol, 89.2%) as a solid.

LCMS: rt 1.31 min. [M+H]⁺ 186.9 m/z.

### 7-(2-(4-chloro-3-fluorophenyl)-1-hydroxy-2-oxoethyl)-6,7-dihydro-1,7-naphthyridin-8(5H)-one

A slurry of 6,7-dihydro-1,7-naphthyridin-8(5H)-one (1.0 g, 1 Eq, 6.7 mmol) and 1-(4-chloro-3-fluorophenyl)-2,2-dihydroxyethan-1-one (1.5 g, 1.1 Eq, 7.4 mmol) in dioxane (20 mL) was heated at 90° C. for 7 hours. The reaction was cooled to room temperature and diluted with Et₂O (30 mL). The resulting precipitate was collected by vacuum filtration, washed with Et₂O, and dried under vacuum at 50° C. to give 7-(2-(4-chloro-3-fluorophenyl)-1-hydroxy-2-oxoethyl)-6,7-dihydro-1,7-naphthyridin-8(5H)-one (1.97 g, 5.89 mmol, 87%) as a white solid.

¹H NMR (400 MHz, CHLOROFORM-d) d ppm 8.72 (d, J=4.16 Hz, 1H), 8.00 (d, J=8.80 Hz, 1H), 7.95 (d, J=9.29 Hz, 1H), 7.44-7.61 (m, 2H), 7.38 (dd, J=7.83, 4.65 Hz, 1H), 7.23 (s, 1H), 3.63 (ddd, J=12.65, 8.25, 4.77 Hz, 1H), 3.24 (ddd, J=12.47, 7.46, 4.77 Hz, 1H), 2.91-3.11 (m, 1H), 2.78-2.90 (m, 1H).

LCMS: rt 1.83 min. [M+H]⁺ 335.0 m/z.

### 7-(5-(4-chloro-3-fluorophenyl)-2-(8-fluoroquinolin-3-yl)oxazol-4-yl)-6,7-dihydro-1,7-naphthyridin-8(5H)-one, Compound 814

To a slurry of 7-(2-(4-chloro-3-fluorophenyl)-1-hydroxy-2-oxoethyl)-6,7-dihydro-1,7-naphthyridin-8(5H)-one (50.0 mg, 1 Eq, 149 μmol) and 8-fluoroquinoline-3-carbonitrile (28.3 mg, 1.1 Eq, 164 μmol) in DCE (2.00 mL) was added triflic acid (112 mg, 66.3 μL, 5.0 Eq, 747 μmol). The resulting cloudy reaction mixture was stirred at 130° C. for 1 hour. The reaction was cooled to room temperature and concentrated to a sticky brown solid. The residue was dissolved in DCM (30 mL) and washed with saturated aqueous NaHCO₃. The aqueous layer was extracted with DCM (30 mL). The combined organics were washed with brine, dried over MgSO₄, filtered, and concentrated to a yellow solid. The crude material was triturated with hot MeOH and the resulting precipitate was collected by vacuum filtration. The filtrate was washed with MeOH and dried under vacuum to give 7-(5-(4-chloro-3-fluorophenyl)-2-(8-fluoroquinolin-3-yl)oxazol-4-yl)-6,7-dihydro-1,7-naphthyridin-8(5H)-one (30 mg, 61 μmol, 41%) as white solid.

¹H NMR: (400 MHz, DMSO-d₆) d ppm 8.70 (d, J=4.0 Hz, 1H), 8.61 (d, J=1.6 Hz, 1H), 8.30 (s, 1H), 8.06 (t, J=5.2 Hz, 1H), 7.94 (d, J=7.6 Hz, 1H), 7.89 (d, J=9.6 Hz, 1H), 7.76-7.71 (m, 3H), 7.64-7.61 (m, 2H), 4.16 (t, J=5.6 Hz, 2H), 3.36-3.34 (m, 2H).

LCMS: rt 2.68 min. [M+H]⁺ 489.0 m/Z.

### Synthesis of Compound 815

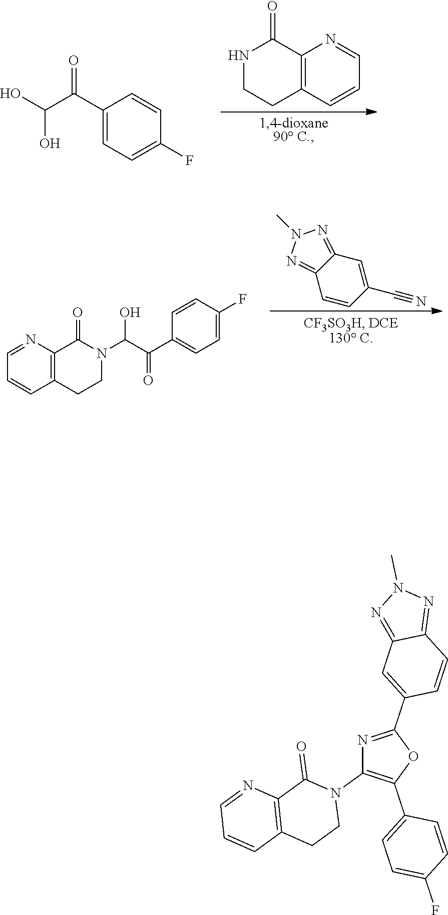

### 7-(2-(4-fluorophenyl)-1-hydroxy-2-oxoethyl)-6,7-dihydro-1,7-naphthyridin-8(5H)-one

A slurry of 5,6,7,8-tetrahydro-1,7-naphthyridin-8-one (1.0 g, 1 Eq, 6.7 mmol) and 2-(4-fluorophenyl)-2-oxoacetaldehyde (1.1 g, 1.1 Eq, 7.4 mmol) in 1,4-dioxane (40 mL) was heated at 90° C. for 17 hours. The reaction was filtered hot through a pad of celite and concentrated to a brown viscous oil. The crude material was recrystallized from EtOAc/heptane with stirring while cooling to room temperature. The solid was collected by vacuum filtration and dried under vacuum to give 7-(2-(4-fluorophenyl)-1-hydroxy-2-oxoethyl)-6,7-dihydro-1,7-naphthyridin-8(5H)-one (1.67 g, 5.56 mmol, 82%) as white solid.

¹H NMR (400 MHz, DMSO-d₆) δ ppm 8.59 (d, J=3.67 Hz, 1H), 8.11 (dd, J=8.80, 5.62 Hz, 2H), 7.76 (d, J=7.58 Hz, 1H), 7.45-7.54 (m, 1H), 7.37 (t, J=8.80 Hz, 2H), 6.95 (q, J=7.09 Hz, 2H), 3.64 (ddd, J=12.59, 7.34, 5.01 Hz, 1H), 3.38 (td, J=8.44, 4.16 Hz, 1H), 2.93-3.05 (m, 1H), 2.80-2.92 (m, 1H). LCMS: rt 1.66 min. [M+H]⁺ 301.1 m/Z.

### 7-(5-(4-fluorophenyl)-2-(2-methyl-2H-benzo[d][1,2,3]triazol-5-yl)oxazol-4-yl)-6,7-dihydro-1,7-naphthyridin-8(5H)-one. Compound 815

7-(5-(4-fluorophenyl)-2-(2-methyl-2H-benzo[d][1,2,3]triazol-5-yl)oxazol-4-yl)-6,7-dihydro-1,7-naphthyridin-8(5H)-one was prepared similar to 7-(5-(4-chloro-3-fluorophenyl)-2-(8-fluoroquinolin-3-yl)oxazol-4-yl)-6,7-dihydro-1,7-naphthyridin-8(5H)-one.

¹H NMR: (400 MHz, DMSO-d₆) δ 8.73 (s, 1H), 8.69 (d, J=4.4 Hz, 1H), 8.13-8.12 (m, 2H), 7.92 (d, J=7.2 Hz, 1H), 7.88 (dd, J=8.8 Hz, 6.0 Hz, 2H), 7.60 (dd, J=7.6 Hz, 4.8 Hz, 1H), 7.35 (t, J=8.8 Hz, 2H), 4.58 (s, 3H), 4.08 (t, J=6.4 Hz, 2H), 3.30 (m, 2H).

LCMS: rt 2.38 min. [M+H]⁺ 441.2 m/z.

### Synthesis of Compounds 816 & 817

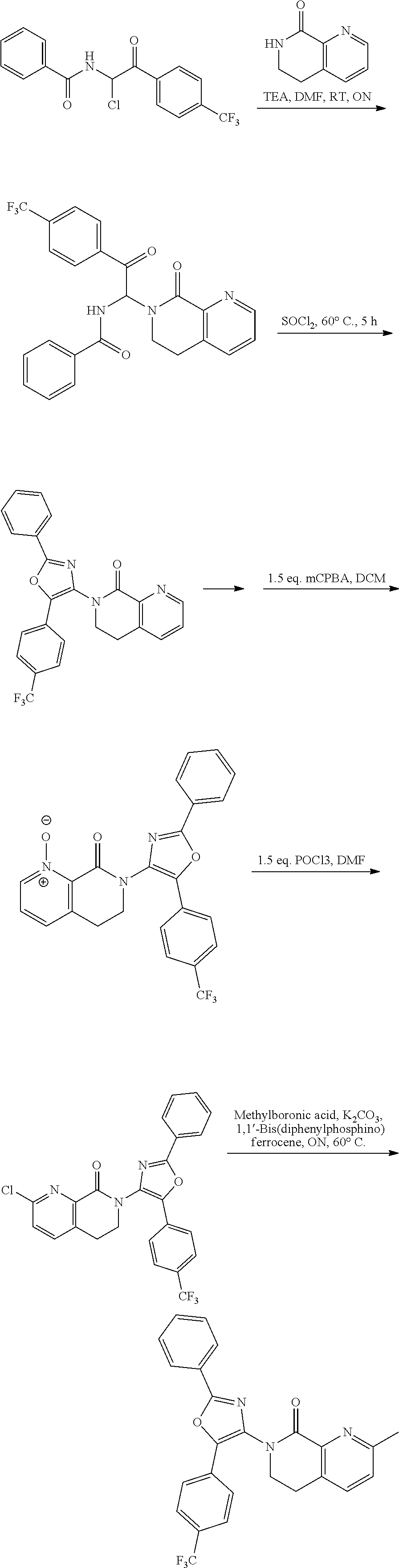

### N-[2-oxo-1-(8-oxo-5,6,7,8-tetrahydro-1,7-naphthyridin-7-yl)-2-[4-(trifluoromethyl)phenyl]ethyl]benzamide

6,7-Dihydro-1.7-naphtyridin-8(5H)one (0.32 g, 2.19 mmol, 1.0 eq) was dissolved in DMF

(3.0 mL) and triethylamine (1.54 mL, 11.0 mmol, 5.0 eq) was added. The mixture was stirred for 30 minutes at room temperature, before N-{1-chloro-2-oxo-2-[4-(trifluoromethyl)phenyl]ethyl}benzamide (0.75 g, 2.19 mmol, 1.0 eq) was added. The mixture was stirred at room temperature for 2 hours. The solvent was then evaporated. Water was added and the resulting precipitate was collected by vacuum filtration and washed with water and diethyl ether to afford N-[2-oxo-1-(8-oxo-5,6,7,8-tetrahydro-1,7-naphthyridin-7-yl)-2-[4-(trifluoromethyl)phenyl]ethyl]benzamide (0.166 g, 0.33 mmol, 15%).

UPLC-MS: RT=4.00 min, [M+H]⁺=454.10, 92%.

### 7-{2-phenyl-5-[4-(trifluoromethyl)phenyl]-1,3-oxazol-4-yl}-5,6,7,8-tetrahydro-1,7-naphthyridin-8-one. Compound 534

N-[2-oxo-1-(8-oxo-5,6,7,8-tetrahydro-1,7-naphthyridin-7-yl)-2-[4-(trifluoromethyl)phenyl]ethyl]benzamide (0.166 g, 0.33 mmol, 1.0 eq.) was dissolved in thionyl chloride (0.93 mL, 12.8 mmol, 32.0 eq) and stirred at 60° C. for 5 hours. The mixture was quenched with methanol upon cooling to room temperature. The resulting solid was collected by vacuum filtration, washed with methanol and diethyl ether, and dried under vacuum to afford 7-{2-phenyl-5-[4-(trifluoromethyl)phenyl]-1,3-oxazol-4-yl}-5,6,7,8-tetrahydro-1,7-naphthyridin-8-one (0.075 g, 0.17 mmol, 47%).

¹H NMR (300 MHz, DMSO-d6) δ ppm 8.68 (dd, J=4.7, 1.6 Hz, 1H), 8.20-8.12 (m, 2H), 7.94 (td, J=4.9, 2.5 Hz, 3H), 7.84 (d, J=8.3 Hz, 2H), 7.70-7.54 (m, 4H), 4.11 (t, J=6.4 Hz, 2H), 3.31 (s, 2H).

UPLC-MS: RT=3.04 min, [M+H]⁺=435.9, 99.9%.

### 8-oxo-7-{2-phenyl-5-[4-(trifluoromethyl)phenyl]-1,3-oxazol-4-yl}-5,6,7,8-tetrahydro-1,7-naphthyridin-1-ium-1-olate

7-{2-phenyl-5-[4-(trifluoromethyl)phenyl]-1,3-oxazol-4-yl}-5,6,7,8-tetrahydro-1,7-naphthyridin-8-one (0.117 g, 0.269 mmol, 1.0 eq) in ethyl acetate (1.17 mL) was cooled to 0° C. To the cold solution was added a solution of 3-chloroperbenzoic acid (0.073 g, 0.326 mmol, 1.21 eq) in ethyl acetate (1.67 mL) over a period of 1.5 hours. The resulting solution was warmed to room temperature and allowed to stir for 3 hours. The reaction mixture was cooled to 0° C. and the resulting slurry was filtered to collect the crude product. This solid was washed with ethyl acetate and dried under vacuum to afford 8-oxo-7-{2-phenyl-5-[4-(trifluoromethyl)phenyl]-1,3-oxazol-4-yl}-5,6,7,8-tetrahydro-1,7-naphthyridin-1-ium-1-olate (0.078 g, 0.14 mmol, 65%).

UPLC-MS: RT=3.50 min, [M+H]⁺=452.05, 83%

### 2-chloro-7-{2-phenyl-5-[4-(trifluoromethyl)phenyl]-1,3-oxazol-4-yl}-5,6,7,8-tetrahydro-1,7-naphthyridin-8-one. Compound 816

8-Oxo-7-{2-phenyl-5-[4-(trifluoromethyl)phenyl]-1,3-oxazol-4-yl}-5,6,7,8-tetrahydro-1,7-naphthyridin-1-ium-1-olate (0.078 g, 0.173 mmol, 1.0 eq) was dissolved in phosphorus(V) oxychloride (0.024 mL, 0.258 mmol, 1.49 eq) and anhydrous DMF (0.84 mL). The reaction was then heated to 110° C. under argon for 16 hours. Upon cooling, the solvent was evaporated to afford 2-chloro-7-{2-phenyl-5-[4-(trifluoromethyl)phenyl]-1,3-oxazol-4-yl}-5,6,7,8-tetrahydro-1,7-naphthyridin-8-one (0.066 g, 0,093 mmol, 54%) as a crude material, which was used for the next step without further purification.

UPLC-MS: RT=4 20 min, [M+H]⁺=470.05, 66%

### 2-methyl-7-{2-phenyl-5-[4-(trifluoromethyl)phenyl]-1,3-oxazol-4-yl}-5,6,7,8-tetrahydro-1,7-naphthyridin-8-one. Compound 817

2-Chloro-7-{2-phenyl-5-[4-(trifluoromethyl)phenyl]-1,3-oxazol-4-yl}-5,6,7,8-tetrahydro-1,7-naphthyridin-8-one (0.066 g, 0.14 mmol, 1.0 eq.), methylboronic acid (0.043 g, 0.704 mmol, 5.01 eq), and potassium carbonate (0.058 g, 0.42 mmol, 2.99 eq.) were dissolved in anhydrous dioxane (2.64 mL) under argon. 1,1′-Bis(diphenylphosphino)ferrocene palladium (II) dichloride (0.01 g, 0.014 mmol, 0.097 eq.) was added, and the mixture was stirred at 90° C. for 16 hours. The reaction mixture was diluted with ethyl acetate and washed with brine (3 times). The organic layer was dried over sodium sulfate and concentrated to afford crude material. The residue was purified via preparative-TLC (3% MeOH in DCM, eluted 3 times) to afford 2-methyl-7-{2-phenyl-5-[4-(trifluoromethyl)phenyl]-1,3-oxazol-4-yl}-5,6,7,8-tetrahydro-1,7-naphthyridin-8-one (0.013 g, 0.029 mmol, 20%) as a white solid.

¹H NMR (300 MHz, DMSO-d₆) δ ppm 8.21-8.11 (m, 2H), 8.00-7.77 (m, 5H), 7.74-7.54 (m, 3H), 7.47 (d, J=7.9 Hz, 1H), 4.09 (t, J=6.4 Hz, 2H), 3.31 (s, 3H), 3.26 (t, J=6.4 Hz, 2H).

UPLC-MS: RT=3.13 min, [M+H]⁺=451.0, 97.9%.

### Synthesis of Compound 818

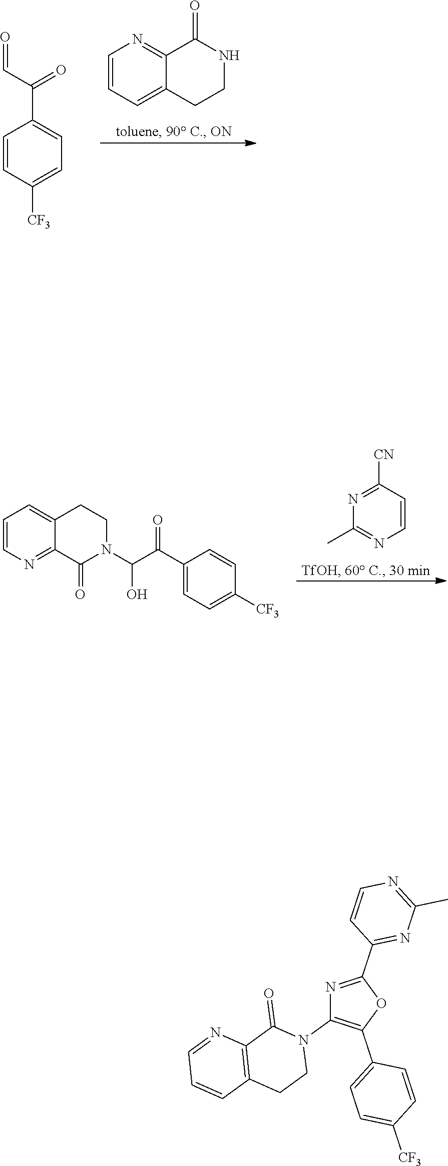

### 7-{1-hydroxy-2-oxo-2-[4-(trifluoromethyl)phenyl]ethyl}-5,6,7,8-tetrahydro-1,7-naphthyridin-8-one

A mixture of 6,7-dihydro-1,7-naphthyridin-8(5H)-one (0.60 g, 4.05 mmol, 1.0 eq) and 2-oxo-2-[4-(trifluoromethyl)phenyl]acetaldehyde hydrate (0.981 g, 4.45 mmol, 1.1 eq) in anhydrous toluene (18.0 mL) was heated for 16 hours at 90° C. The reaction mixture was cooled and concentrated under reduced pressure to obtain a crude material. This material was dissolved in dichloromethane and hexanes was added. The obtained suspension was stirred at room temperature for 20 minutes. A precipitate formed and was collected by vacuum filtration. The material was washed with hexanes and dried under vacuum to give 7-{1-hydroxy-2-oxo-2-[4-(trifluoromethyl)phenyl]ethyl}-5,6,7,8-tetrahydro-1,7-naphthyridin-8-one (1.0 g, 2.48 mmol, 61%).

UPLC-MS: RT=1.83 min, [M+H]⁺=351.10, 87%.

### 7-[2-(2-methylpyrimidin-4-yl)-5-[4-(trifluoromethyl)phenyl]-1,3-oxazol-4-yl]-5,6,7,8-tetrahydro-1,7-naphthyridin-8-one. Compound 818

To a slurry of 7-{1-hydroxy-2-oxo-2-[4-(trifluoromethyl)phenyl]ethyl}-5,6,7,8-tetrahydro-1,7-naphthyridin-8-one (1.0 g, 2.48 mmol, 1.0 eq) and 2-methylpyrimidine-4-carbonitrile (0.355 g, 2.98 mmol, 1.2 eq) in 1,2-dichloroethane (40.0 mL) was added trifluoromethanesulfonic acid (1.86 g, 12.4 mmol, 5.0 eq). The resulting cloudy reaction mixture was stirred at 100° C. for 1 hour. The reaction mixture was diluted with dichloromethane and washed with a saturated aqueous solution of sodium bicarbonate (3 times). The organic layer was washed with brine, dried over sodium sulfate, and concentrated. The resulting crude material was purified by silica gel column chromatography (0-2% MeOH in DCM). The fractions containing product were concentrated and then treated with ethyl acetate resulting in a precipitate forming. This precipitate was collected by vacuum filtration, washed with cold ethyl acetate, and dried under vacuum to give 7-≡-[2-(2-methylpyrimidin-4-yl)-5-[4-(trifluoromethyl)phenyl]-1,3-oxazol-4-yl]-5,6,7,8-tetrahydro-1,7-naphthyridin-8-one (0.24 g, 0.521 mmol, 21%).

¹H NMR (300 MHz, DMSO-d6) δ ppm 8.97 (d, J=5.2 Hz, 1H), 8.68 (dd, J=4.6, 1.6 Hz, 1H), 8.05 (d, J=5.2 Hz, 1H), 8.01-7.90 (m, 3H), 7.88 (d, J=8.4 Hz, 2H), 7.62 (dd, J=7.8, 4.6 Hz, 1H), 4.14 (t, J=6.4 Hz, 2H), 3.36 (t, 2H), 2.77 (s, 3H).

UPLC-MS: RT=2.63 min, [M+H]⁺=452.2, 99.8%.

### Synthesis of Compound 819

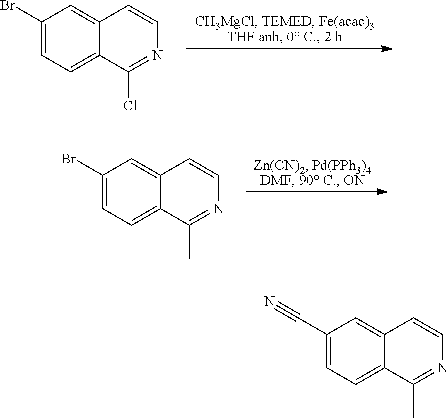

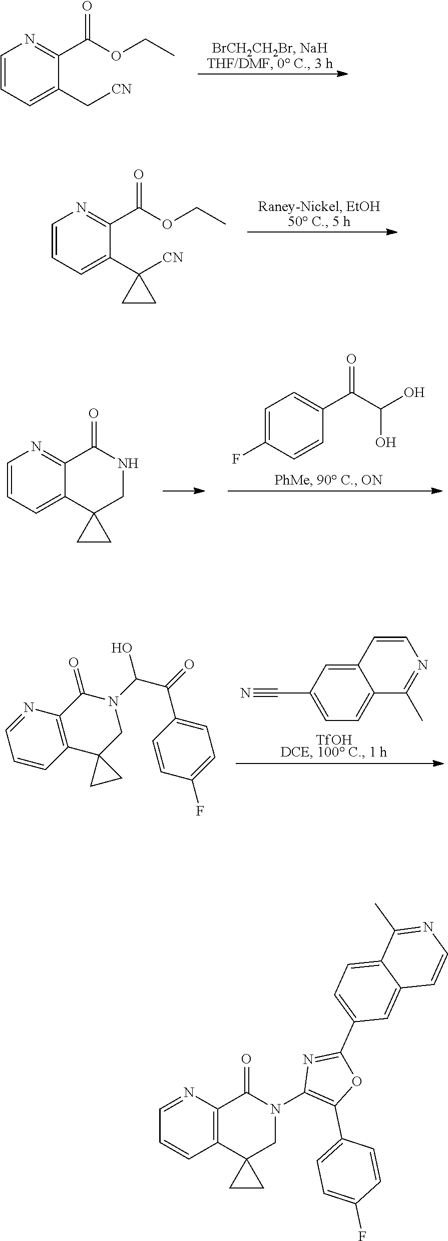

### 6-bromo-1-methylisoquinoline

6-Bromo-1-chloroisoquinoline (3.0 g, 12.4 mmol, 1.0 eq) was dissolved in anhydrous THF (30.0 mL) and N,N,N′,N′-tetramethyl ethylenediamine (0.928 mL, 6.19 mmol, 0.5 eq) was added followed by iron(III) acetylacetonate (0.044 g, 0.124 mmol, 0.01 eq). The resulting mixture was cooled to 0° C. and methyl magnesium chloride (3 M in THF, 5.36 mL, 16.1 mmol, 1.3 eq) was added. The reaction was stirred for 2 hours at 0° C. Upon warming to room temperature, the reaction mixture was quenched with 5% aqueous citric acid. Ethyl acetate was added, and the layers were separated. The organic layer was washed with brine, dried over sodium sulfate, and concentrated under reduced pressure. The crude material was purified via silica gel column chromatography (30-60% EtOAc in hexanes) to give 6-bromo-1-methylisoquinoline (1.14 g, 4.93 mmol, 60%) as a solid.

¹H NMR (300 MHz, DMSO-d6) δ ppm 8.38 (d, J=5.8 Hz, 1H), 8.25 (d, J=2.1 Hz, 1H), 8.15 (dt, J=8.9, 0.7 Hz, 1H), 7.79 (dd, J=8.9, 2.1 Hz, 1H), 7.65 (d, J=5.8 Hz, 1H), 2.87 (s, 3H).

### 1-methylisoquinoline-6-carbonitrile

6-Bromo-1-methylisoquinoline (1.5 g, 6.08 mmol, 1.0 eq) and zinc cyanide (0.714 g, 6.08 mmol, 1.0 eq) were dissolved in anhydrous DMF (22.5 mL), and the solution was purged with argon for 10 minutes. Then, tetrakis(triphenylphosphine)palladium(0) (0.351 g, 0.304 mmol, 0.05 eq) was added, and the reaction mixture was stirred at 90° C. for 16 hours. Upon cooling, the solvent was evaporated, and the resulting oily residue was directly purified by silica gel column chromatography (0-5% MeOH in DCM) to give 1-methylisoquinoline-6-carbonitrile (0.926 g, 5.23 mmol, 86%) as pale-yellow solid.

¹H NMR (300 MHz, DMSO-d6) δ ppm 8.61 (d, J=1.6 Hz, 1H), 8.50 (d, J=5.7 Hz, 1H), 8.38 (dt, J=8.7, 0.9 Hz, 1H), 7.96 (dd, J=8.7, 1.7 Hz, 1H), 7.78 (d, J=5.8 Hz, 1H), 2.92 (s, 3H).

### ethyl 3-(1-cyanocyclopropyl)pyridine-2-carboxylate

Sodium hydride (0.164 g, 4.10 mmol, 2.0 eq) was suspended in anhydrous THF (4.0 mL). The mixture was cooled to 0° C. and a solution of ethyl 3-(cyanomethyl)pyridine-2-carboxylate (0.39 g, 2.05 mmol, 1.0 eq) in anhydrous DMF (2.0 mL) was added. The resulting mixture was stirred for 15 minutes at 0° C. Then, 1,2-dibromoethane (0.770 g, 2.05 mmol, 2.0 eq) was added slowly. The reaction mixture was stirred for 3 hours at 0° C. The reaction mixture was warmed to room temperature and diluted with ethyl acetate. The organic layer was washed with water 3 times. The organic solution was then dried over sodium sulfate and concentrated. The crude material was purified by silica gel column chromatography (50-80% EtOAc in hexanes) to give ethyl 3-(1-cyanocyclopropyl)pyridine-2-carboxylate (0.160 g, 0.740 mmol, 36%) as a colorless oil.

¹H NMR (300 MHz, DMSO-d6) δ ppm 8.65 (dd, J=4.7, 1.5 Hz, 1H), 8.05 (dd, J=8.0, 1.5 Hz, 1H), 7.62 (dd, J=8.0, 4.7 Hz, 1H), 4.41 (q, J=7.2 Hz, 2H), 1.75-1.67 (m, 2H), 1.43-1.33 (m, 5H).

### 7,8-dihydro-6H-spiro[1,7-naphthyridine-5,1′-cyclopropan]-8-one

A three-necked round-bottom flask equipped with a reflux condenser was charged with ethyl 3-(1-cyanocyclopropyl)pyridine-2-carboxylate (0.160 g, 0.740 mmol, 1.0 eq) dissolved in ethanol (9.6 mL). The reaction mixture was cooled to 0° C. The flask was evacuated and refilled with argon three times. Then, Raney-Nickel (suspension in water, 2 mL) was added. Next, the flask was evacuated and refilled with hydrogen three times. The reaction mixture was then stirred at 50° C. for 4 hours. Upon cooling to room temperature, the crude mixture was filtered through a pad of celite, which was then washed with ethanol. The organic filtrates were concentrated under reduced pressure. The crude material was purified by silica gel column chromatography (10% MeOH in DCM with 3% ammonia) to give 7,8-dihydro-6H-spiro[1,7-naphthyridine-5,1′-cyclopropan]-8-one (0.110 g, 0.631 mmol, 85%) as a colorless oil.

¹H NMR (300 MHz, DMSO-d6) δ ppm 8.54 (dd, J=4.2, 1.7 Hz, 1H), 8.23 (s, 1H), 7.52-7.43 (m, 2H), 3.26 (d, J=2.8 Hz, 2H), 1.12-1.07 (m, 2H), 1.02 (t, J=5.5 Hz, 2H).

### 7-[2-(4-fluorophenyl)-1-hydroxy-2-oxoethyl]-7,8-dihydro-6H-spiro[1,7-naphthyridine-5,1′-cyclopropan]-8-one

7,8-Dihydro-6H-spiro[1,7-naphthyridine-5,1′-cyclopropan]-8-one (0.055 g, 0.253 mmol, 1.0 eq) and 4-fluorophenylglyoxal hydrate (0.047 g, 0.278 mmol, 1.1 eq) were dissolved in anhydrous toluene (1.65 mL). The resulting mixture was heated at 90° C. for 16 hours. The toluene was evaporated to give crude product. The crude material was triturated with EtOAc in hexanes (2/8 v/v) and the resulting suspension was stirred for 15 minutes. The precipitate was collected by vacuum filtration, washed with hexanes, and dried under vacuum to give 7-[2-(4-fluorophenyl)-1-hydroxy-2-oxoethyl]-7,8-dihydro-6H-spiro[1,7-naphthyridine-5,1′-cyclopropan]-8-one (0.0468 g, 0.149 mmol, 59%) as yellow solid.

¹H NMR (300 MHz, DMSO-d6) δ ppm 8.55 (dd, J=3.7, 2.0 Hz, 1H), 8.10 (dd, J=8.7, 5.7 Hz, 2H), 7.53-7.43 (m, 2H), 7.37 (t, J=8.8 Hz, 2H), 6.99-6.88 (m, 2H), 3.30 (m, 2H), 1.19-1.01 (m, 3H), 0.78-0.64 (m, 1H).

### 7-[5-(4-fluorophenyl)-2-(1-methylisoquinolin-6-yl)-1,3-oxazol-4-yl]-7,8-dihydro-6H-spiro[1,7-naphthyridine-5,1′-cyclopropan]-8-one. Compound 819

7-[2-(4-Fluorophenyl)-1-hydroxy-2-oxoethyl]-7,8-dihydro-6H-spiro[1,7-naphthyridine-5,1′-cyclopropan]-8-one (0.047 g, 0.149 mmol, 1.0 eq) and 1-methylisoquinoline-6-carbonitrile (0.030 g, 0.179 mmol, 1.2 eq) were dissolved in 1,2-dichloroethane (1.87 mL). Trifluoromethanesulfonic acid (0.075 g, 0.502 mmol, 5.0 eq) was added and the reaction mixture was stirred at 100° C. for 1 hour. Upon cooling, the solvent was evaporated. The residue was dissolved in dichloromethane and washed with saturated aqueous sodium bicarbonate. The aqueous layer was extracted with dichloromethane. The organic layers were combined and washed with brine. The organic layer was dried over sodium sulfate and concentrated to give the crude material. The crude product was purified via prep-HPLC (C18 column, 10-100% MeCN in water with 0.1% TFA) to give 7-[5-(4-fluorophenyl)-2-(1-methylisoquinolin-6-yl)-1,3-oxazol-4-yl]-7,8-dihydro-6H-spiro[1,7-naphthyridine-5,1′-cyclopropan]-8-one (0.015 g, 0.0315 mmol, 21%) as a

¹H NMR (400 MHz, DMSO-d6) δ ppm 8.76 (d, J=1.5 Hz, 1H), 8.64 (dd, J=4.4, 1.5 Hz, 1H), 8.46 (d, J=5.7 Hz, 1H), 8.41 (s, 1H), 8.31 (dd, J=8.9, 1.6 Hz, 1H), 7.89 (d, J=1.9 Hz, 1H), 7.88-7.84 (m, 2H), 7.66 (dd, J=8.0, 1.5 Hz, 1H), 7.59 (dd, J=8.0, 4.5 Hz, 1H), 7.40 (t, J=8.9 Hz, 2H), 3.97 (s, 2H), 2.94 (s, 3H), 1.33-1.27 (m, 2H), 1.21-1.15 (m, 2H).

UPLC-MS: RT=2.42 min, [M+H]⁺=477.2, 99.7%.

### Example 6

### Cryo-EM Methods

Tau PHF filament purification. Filament purification was based on Fitzpatrick et al., 2017¹. Briefly, 5 g of fresh-frozen frontal cortex tissue from a patient with Alzheimer's disease was homogenized at 10 mL/g of tissue in 10 mM Tris-HCl (pH 7.4), 800 mM NaCl, 1 mM EGTA, and 10% sucrose. The homogenate was centrifuged at 20,000 g for 10 minutes, and the supernatant was kept. The pellets were resuspended in 5 volumes of the same buffer, centrifuged again, and the 2 supernatants were combined. A final concentration of 1% N-laurosarcosinate (w/v) was added to the combined supernatant, and this mixture was incubated for 1 h at room temperature. It was then centrifuged at 100,000 g for 1 hour, and the pellets were resuspended in 30 volumes of 10 mM Tris-HCl (pH 7.4), 800 mM NaCl, 1 mM EGTA, 5 mM EDTA, and 10% sucrose. This was followed by another centrifugation at 20,100 g for 30 minutes at 4° C. The supernatant was kept and centrifuged at 100,000 g for 1 hour, and the final pellet resuspended in 20 mM Tris-HCl (pH 7.4) and 100 mM NaCl at a concentration of 10 μL/g frozen tissue.

MSA filament purification. Filament purification was based on Schweigenhauser et al., 2020². Briefly, 1-2 g of fresh-frozen tissue from the cerebellum of patients clinically and neuropathologically diagnosed with MSA was homogenized at 20 mL/g of tissue in 10 mM Tris-HCl (pH 7.4), 800 mM NaCl, 1 mM EGTA, and 10% sucrose. A final concentration of 1% N-laurosarcosinate (w/v) was added to the homogenates, and these mixtures were incubated at 37° C. for 30 minutes. Homogenates were then centrifuged at 10,000 g for 10 minutes, and the supernatant was kept and then centrifuged at 100,000 g for 20 minutes. The pellets were resuspended at 500 μL/g of frozen tissue in 10 mM Tris-HCl (pH 7.4), 800 mM NaCl, 1 mM EGTA, and 10% sucrose. This was followed by another centrifugation at 3,000 g for 5 minutes. The supernatant was kept and diluted threefold with 50 mM Tris-HCl (pH 7.4), 150 mM NaCl, 10% sucrose, and 2% sarkosyl, followed by another centrifugation at 150,000 g for 1 hour. The final pellet resuspended in 30 mM Tris-HCl (pH 7.4) at a concentration of 100 μL/g frozen tissue.

Cryo-EM sample preparation and data collection. Purified frontal cortex and cerebellum tissue was incubated with 20 μM ligand for 45 minutes prior to freezing. For the tau PHF, 3 μL of purified filaments mixed with GTP-1 was added to a glow-discharged 200 mesh 1.2/1.3R Au Quantifoil grid for 10 seconds before blotting for 2 seconds, and then a second 3 μL aliquot was added for 3 seconds and blotted for 1 second before being plunge frozen in liquid ethane using a FEI Vitrobot Mark IV (Thermo Fisher Scientific). For the synuclein ligands, 3 μL of purified filaments mixed with compound was added to a 200 mesh 1.2/1.3R Au Quantifoil grid coated with a 2 nm thick layer of carbon, which had not been glow discharged. After 30 seconds, grids were blotted for 7.5 seconds at room temperature and 100% humidity. Grids were plunge frozen in liquid ethane using a FEI Vitrobot Mark IV. For all samples, super-resolution movies were collected at a nominal magnification of 105,000× (physical pixel size: 0.417 Å per pixel) on a Titan Krios (Thermo Fisher Scientific) operated at 300 kV and equipped with a K3 direct electron detector and BioQuantum energy filter (Gatan, Inc.) set to a slit width of 20 eV. A defocus range of 0.8 to 1.8 μm was used with a total exposure time of 2.024 seconds fractionated into 0.025 seconds subframes. The total dose for each movie was 46 electrons/Å². Movies were motion-corrected using MotionCor2³ in Scipion⁴ and were Fourier cropped by a factor of 2 to a final pixel size of 0.834 Å per pixel.

Image processing. All image processing was done in RELION 4⁵, and the same workflow was used for each dataset. Dose-weighted summed micrographs were imported into RELION 4. The contrast transfer function was estimated using CTFFIND-4.1⁶. Filaments were manually picked and then segments were extracted with a box size of 900 pixels downscaled to 300 pixels. Contaminants and segments contributing to straight filaments were separated out using reference-free 2D class averaging. The remaining segments were re-extracted with a box size of 288 pixels without downscaling. An initial model was generated from a cylinder in RELION. One or more rounds of 3D classification with image alignment were performed, with helical rise and tilt parameters fixed to eliminate obvious junk particles. One or more rounds of 3D auto-refinement was run using the best map from 3D classification low-pass filtered to 10 Å, allowing rise and twist parameters to vary. Maps were sharpened using the standard post-processing procedures in RELION. Full statistics are shown in Table 1.

Model building, refinement, and analysis. Prior to ligand placement, a single strand of a previously solved MSA or tau PHF filament structure² (PDB: 6XYQ) was refined against the density using Phenix⁷. Refinement of the ligand binding pocket was done in COOT⁸. Ligands were placed by hand in the density using Chimera⁹. This model was then translated to give a stack spanning 5 rungs. Refinement statistics are shown in Table 1.

TABLE 1
Refinement statistics for Cryo-EM experiments

| | Tau | | | | |
|---|---|---|---|---|---|
| Data collection | PHF + | MSA | MSA + | MSA + | MSA + |
| and processing | GTP-1 | apo | 79 | 125 | 83 |
| Microscope and | Titan Krios, K3 | | | | |
| camera | | | | | |
| Magnification | 105,000 | | | | |
| Voltage (kV) | 300 | | | | |
| Electron | 46 | | | | |
| exposure (e⁻/Å²) | | | | | |
| Dose rate | 16 | | | | |
| (e⁻/physical | | | | | |
| pixel/sec) | | | | | |
| Exposure per | 0.024 | | | | |
| frame (sec) | | | | | |
| Defocus range | −0.8 to −1.8 | | | | |
| (μm) | | | | | |
| Physical pixel | 0.834 | | | | |
| size (Å) | | | | | |
| Movies collected | 15,160 | 49,323 | 24,139 | 29,568 | 70,780 |
| Box size (pixels) | 288 | 288 | 288 | 288 | 288 |
| Interbox distance | 28 | 28 | 28 | 28 | 28 |
| (Å) | | | | | |
| Initial segments | 380,428 | 140,872 | 113,078 | 133,072 | 125,050 |
| extracted | | | | | |
| Final segments | 30,199 | 15,261 | 39,119 | 16,916 | 11,706 |
| Resolution (Å) | 2.70 | 3.04 | 2.96 | 3.00 | 3.33 |
| B-factor (Å²) | −42.9 | −61.4 | −57.0 | −42.5 | −84.7 |
| Helical rise (Å) | 2.37 | 4.74 | 4.73 | 4.73 | 4.74 |
| Helical twist (°) | 179.45 | −1.37 | −1.42 | −1.42 | −1.33 |
| Refinement | | | | | |
| Model | | | | | |
| Composition | | | | | |
| Non-hydrogen | 5,810 | 5,004 | 5,665 | 5,658 | 5,171 |
| atoms | | | | | |
| Protein residues | 730 | 730 | 800 | 800 | 730 |
| Ligands | 10 | 0 | 5 | 5 | 5 |
| R.M.S. | | | | | |
| Deviations | | | | | |
| Bond Lengths (Å) | 0.004 | 0.004 | 0.008 | 0.008 | 0.007 |
| Bond angles (°) | 0.988 | 0.513 | 1.081 | 1.099 | 0.904 |
| Validation | | | | | |
| Molprobity score | 2.30 | 1.96 | 2.31 | 2.17 | 2.64 |
| Clashscore | 6.91 | 16.34 | 12.68 | 8.15 | 14.35 |
| Rotamer outliers (%) | 3.10 | 0.00 | 2.78 | 2.78 | 3.42 |
| C_{β} outliers (%) | 4.35 | 0.00 | 0.00 | 0.00 | 0.00 |
| Ramachandran | | | | | |
| Plot | | | | | |
| Favored (%) | 90.14 | 96.34 | 94.74 | 94.23 | 93.10 |
| Allowed (%) | 9.86 | 3.66 | 5.26 | 5.13 | 6.20 |
| Outliers (%) | 0.00 | 0.00 | 0.00 | 0.64 | 0.70 |
| Data collection | MSA + | MSA + | MSA + | MSA + | MSA + |
| and processing | 427 | 22 | 293 | 294 | 149 |
| Microscope and | Titan Krios, K3 | | | | |
| camera | | | | | |
| Magnification | 105,000 | | | | |
| Voltage (kV) | 300 | | | | |
| Electron | 46 | | | | |
| exposure (e⁻/Å²) | | | | | |
| Dose rate | 16 | | | | |
| (e⁻/physical | | | | | |
| pixel/sec) | | | | | |
| Exposure per | 0.024 | | | | |
| frame (sec) | | | | | |
| Defocus range | −0.8 to −1.8 | | | | |
| (μm) | | | | | |
| Physical pixel | 0.834 | | | | |
| size (Å) | | | | | |
| Movies collected | 62,003 | 23,937 | 14,493 | 23,995 | 62,350 |
| Box size (pixels) | 288 | 288 | 288 | 288 | 288 |
| Interbox distance | 28 | 28 | 28 | 28 | 28 |
| (Å) | | | | | |
| Initial segments | 135,293 | 187,228 | 148.638 | 174,514 | 245,634 |
| extracted | | | | | |
| Final segments | 24,213 | 22,186 | 33,222 | 47,547 | 30,741 |
| Resolution (Å) | 3.33 | 2.92 | 3.11 | 2.82 | 3.29 |
| B-factor (Å²) | −77.8 | −65.8 | −68.2 | −71.5 | −85.5 |
| Helical rise (Å) | 4.76 | 4.76 | 4.76 | 4.76 | 4.76 |
| Helical twist (°) | −1.33 | −1.30 | −1.32 | −1.33 | −1.31 |
| Refinement | | | | | |
| Model | | | | | |
| Composition | | | | | |
| Non-hydrogen | 5,143 | 5,185 | 5,160 | 5,220 | 5,355 |
| atoms | | | | | |
| Protein residues | 730 | 730 | 730 | 735 | 730 |
| Ligands | 4 | 5 | 5 | 5 | 10 |
| R.M.S. | | | | | |
| Deviations | | | | | |
| Bond Lengths (Å) | 0.009 | 0.003 | 0.004 | 0.004 | 0.005 |
| Bond angles (°) | 1.277 | 0.530 | 0.505 | 0.779 | 0.977 |
| Validation | | | | | |
| Molprobity score | 2.60 | 2.31 | 2.11 | 2.29 | 2.19 |
| Clashscore | 20.28 | 13.10 | 16.55 | 18.62 | 18.99 |
| Rotamer outliers (%) | 3.03 | 5.05 | 3.03 | 4.44 | 3.23 |
| C_{β} outliers (%) | 0.00 | 0.00 | 0.00 | 0.00 | 0.00 |
| Ramachandran | | | | | |
| Plot | | | | | |
| Favored (%) | 93.38 | 97.18 | 97.89 | 97.89 | 97.89 |
| Allowed (%) | 6.62 | 2.82 | 2.11 | 2.11 | 2.11 |
| Outliers (%) | 0.00 | 0.00 | 0.00 | 0.00 | 0.00 |

### Example 7

Autoradiography Imaging of Frozen Brain Sections Using Tritiated Compounds. 12 um sagittal sections of fresh frozen brains are cut on the cryostat, placed on superfrost plus microscope slides, and stored at −80° C. until needed. When needed, slides are removed from the −80° C. freezer and allowed to thaw and completely dry at room temp for ˜5-10 min. The sections are then incubated in PBS for 30 min. at room temperature. Tritiated compounds are then diluted in PBS to the desired concentration and the slides are incubated with the compounds for 90 minutes at room temperature with gentle shaking. Following incubation, the slides are rinsed in ice-cold PBS two time for 20 minutes each with gentle shaking. They are then dipped 3 times in ice-cold water and allowed to dry in a dessicator. Once completely dry, the slides are laid directly onto a Tritium Phosphor Screen and incubated in an exposure cassette for 3-5 days at room temperature. The screen is then removed and imaged in an Amersham Typhoon biomolecular image.

pSyn Immunofluorescence Imaging of Frozen Brain Sections Using EP1536Y Protocol. Frozen hemi-brains were cryosectioned at 20 μm, then fixed in 4% paraformaldehyde. Sections were blocked with 10% normal goat serum (Vector laboratories, S-1000), then incubated with primary antibody Anti-Alpha-synuclein (phospho 5129) antibody [EP1536Y], (Abcam ab51253) 1:1000 for two hours at room temp. After washing, sections were incubated in secondary antibody Alexa Fluor goat anti rabbit 488 (Thermo Fisher A11008) 1:500 for two hours. Sections were then washed and incubated in Hoechst (Life Technologies H3570) 1:5000 for 10 minutes and then rinsed with DI water and coverslipped using Permafluor aqueous mounting medium (Thermo Scientific, TA030FM). Slides were imaged using the Zeiss AxioScan.Z1

### Example 8

The permeability of select compounds was tested and the data can be found in Table 2.

Parallel Artificial Membrane Permeability Assay (PAMPA). 5 uL of 1% lecithin (VWR 0579) dissolved in n-dodecane (Sigma Aldrich D221104) was applied to each well of a MultiScreen IP PAMPA filter plate (Millipore MAIPSWU10) and allowed to equilibrate for 5 minutes. 300 uL of PBS was added to the acceptor plate and 150 uL of 200 uM test compound dissolved in PBS and 30% ethanol was added to the donor wells before incubating the donor plate in the acceptor plate for 4 hr at 500 rpm. 100 uL was sampled from the donor and acceptor wells and the concentrations were measured by liquid chromatography-mass spectrometry (SCIEX 4500) or UV-VIS plate reader (BMG).

$${P}_{app} = C \times - ln ( 1 - \frac{{C}_{A} ( t )}{{C}_{equil}} ) \\ C = \frac{{V}_{D} \times {V}_{A}}{( {V}_{D} + {V}_{A} ) \times area \times time} \\ {C}_{equil} = \frac{{C}_{D} ( t ) \times {V}_{D} + {C}_{A} ( t ) \times {V}_{A}}{{V}_{D} + {V}_{A}} \\ {V}_{D} - volume donor compartment ( 150 uL ) \\ {V}_{A} - volume acceptor compartment ( 300 uL ) \\ area = 0.28 {cm}^{2}$$

TABLE 2
Permeability of select compounds
Parallel Artificial Membrane
Permeability Assay

| | PAMPA: P_app | |
|---|---|---|
| | (×10e−6 cm/sec) | |
| Compound | pH 5.0 | pH 7.4 |
| 1 | >50.0 | >50.0 |
| 3 | >50.0 | >50.0 |
| 6 | >50.0 | >50.0 |
| 10 | 36.3 | >50.0 |
| 11 | >50.0 | >50.0 |
| 14 | >50.0 | >50.0 |
| 16 | >50.0 | >50.0 |
| 17 | >50.0 | >50.0 |
| 20 | >50.0 | >50.0 |
| 22 | >50.0 | >50.0 |
| 30 | >50.0 | >50.0 |
| 31 | 14.9 | 16.7 |
| 33 | 22.7 | 35.7 |
| 34 | 24.4 | 14.3 |
| 35 | >50.0 | >50.0 |
| 36 | >50.0 | >50.0 |
| 37 | >50.0 | >50.0 |
| 38 | 46.1 | >50.0 |
| 39 | 35.6 | 46.1 |
| 40 | >50.0 | >50.0 |
| 41 | 9.5 | 6.3 |
| 42 | 46.7 | 37 |
| 43 | 27.3 | >50.0 |
| 44 | 48.8 | 40.8 |
| 45 | 47 | >50.0 |
| 47 | 19.7 | 49.7 |
| 48 | 2.2 | 4.5 |
| 50 | 36.3 | >50.0 |
| 51 | >50.0 | >50.0 |
| 52 | >50.0 | >50.0 |
| 54 | 4.9 | 30.2 |
| 55 | 5.7 | 6.2 |
| 56 | 29.1 | >50.0 |
| 57 | >50.0 | >50.0 |
| 63 | >50.0 | >50.0 |
| 66 | >50.0 | >50.0 |
| 67 | >50.0 | >50.0 |
| 69 | >50.0 | >50.0 |
| 72 | >50.0 | >50.0 |
| 73 | 20.2 | 19.8 |
| 74 | >50.0 | >50.0 |
| 75 | 35 | 49.9 |
| 77 | >50.0 | >50.0 |
| 78 | >50.0 | >50.0 |
| 83 | 40.3 | >50.0 |
| 84 | >50.0 | >50.0 |
| 86 | >50.0 | >50.0 |
| 87 | >50.0 | >50.0 |
| 88 | >50.0 | >50.0 |
| 89 | ND | <0.602 |
| 92 | ND | 1.46 |
| 93 | >50.0 | >50.0 |
| 94 | 27.2 | 41.1 |
| 95 | >50.0 | 41.1 |
| 96 | >50.0 | >50.0 |
| 97 | >50.0 | >50.0 |
| 98 | 49.1 | >50.0 |
| 99 | >50.0 | >50.0 |
| 101 | >50.0 | >50.0 |
| 102 | >50.0 | >50.0 |
| 103 | 47.7 | 29.4 |
| 104 | 36 | >50.0 |
| 105 | >50.0 | >50.0 |
| 106 | >50.0 | 39.6 |
| 107 | >50.0 | >50.0 |
| 108 | >50.0 | >50.0 |
| 109 | >50.0 | >50.0 |
| 110 | >50.0 | 47.4 |
| 111 | >50.0 | >50.0 |
| 112 | >50.0 | >50.0 |
| 113 | >50.0 | >50.0 |
| 114 | 36.5 | 32.5 |
| 115 | >50.0 | >50.0 |
| 118 | >50.0 | 48.1 |
| 119 | >50.0 | >50.0 |
| 124 | ND | 5.82 |
| 138 | >50 | >50 |
| 140 | 36.7 | 33.6 |
| 149 | 16.9 | 11.2 |
| 152 | ND | 1.26 |
| 153 | ND | 1.94 |
| 154 | ND | 1.47 |
| 160 | ND | 12.8 |
| 161 | >50 | >50 |
| 163 | >50 | >50 |
| 167 | ND | 11.4 |
| 168 | ND | 6.9 |
| 183 | 45 | 40 |
| 186 | ND | 6.27 |
| 187 | ND | 12.5 |
| 189 | ND | 10.9 |
| 194 | ND | 15.9 |
| 195 | ND | 5.1 |
| 200 | ND | 1.6 |
| 204 | ND | 15.5 |
| 210 | ND | 15.8 |
| 211 | ND | 4.02 |
| 212 | ND | 6.25 |
| 213 | 36 | 48 |
| 214 | 30 | 42 |
| 215 | >50 | >50 |
| 216 | ND | 1.63 |
| 217 | ND | 3.91 |
| 218 | 5.3 | 5.3 |
| 219 | ND | 4.25 |
| 223 | ND | 10 |
| 224 | ND | 7.44 |
| 226 | ND | 2.43 |
| 238 | ND | 2.28 |
| 239 | ND | 0.664 |
| 240 | ND | 2.41 |
| 241 | ND | 1.36 |
| 242 | ND | 5.72 |
| 244 | >50 | 45 |
| 245 | >50 | >50 |
| 246 | >50 | >50 |
| 248 | 46.6 | >50 |
| 249 | 47.1 | >50 |
| 250 | >50 | >50 |
| 252 | >50 | >50 |
| 256 | >50 | >50 |
| 262 | 1.2 | 2 |
| 263 | 17.9 | 20.7 |
| 264 | 27.1 | 26.4 |
| 266 | >50 | >50 |
| 267 | ND | 5.98 |
| 269 | 19.1 | 26.7 |
| 272 | 8.8 | 8.9 |
| 273 | 2.8 | 3.6 |
| 274 | 4.6 | 6.3 |
| 275 | 0.8 | 1.3 |
| 276 | >50 | >50 |
| 277 | >50 | >50 |
| 278 | >50 | >50 |
| 279 | 44.7 | >50 |
| 280 | 19.5 | 19.5 |
| 281 | >50 | >50 |
| 284 | >50 | >50 |
| 288 | >50 | >50 |
| 290 | 16.5 | 39.3 |
| 292 | 7.9 | 13.5 |

### Example 9

Select compounds were counter screened and the data can be found in Table 3.

### Preparation of AD11A Inoculation Material

A fragment of the Middle Tempral Gyrus (MTG) from Alzheimer's Disease (AD) patient 2217 was resuspended in 9 volumes of PBS (ml/g of brain) and homogenized 3×12 seconds using a probe homogenizer (Thomas Scientific) with disposable Omni Tip plastic homogenizing probes (Thomas Scientific Cat No 3409Y77) to generate a 10% brain homogenate (10% AD11-BH). The 10% AD11-BH was clarified of insoluble debris by centrifugation at 800×g for 5 minutes. The resulting supernatant was collected and the protein concentration of this clarified BH (AD11-CBH) was determined by bicinchoninic acid assay (BCA) (Thermo Fisher Cat No. 23227). HEK293 cells stably expressing a C-terminally Clover-tagged truncated 4R-Tau²⁴³⁻³⁸² transgene containing the P301L and V337M mutations and a C-terminally Ruby-tagged truncated 3R-Tau²⁴³⁻³⁸² transgene containing the L266V and V337M mutations (128/139 cells) were transfected with the AD11-CBH using lipofectamine 2000 (LF2K) (Thermo Fisher Cat No. 11668500). Briefly, 0.32 μl of LF2K were diluted in 5 μl Opti-MEM (Thermo Scientific Cat No 31985070) per well of a 384-well plate and incubated for 5 min at room temperature (RT). This mixture was added to 0.32 μg of AD11-CBH diluted in 5 μl of PBS per well of a 384-well plate and vortex briefly. The transfection mixture was incubated at room temperature for 90 min. The transfection complexes were subsequently added to a single well of a 384-well plate containing 3500 128/139 cells. After 3 days the cells were harvested, counted and plated at a density of 1 cell/well in a 96-well plate. A clonal cell line containing Clover and Ruby positive Tau aggregates was selected and termed 416-AD11A cells. Following the expansion of these 416-AD11A cells, a cell lysate was prepared by freeze/thaw. The cells from a confluent T175 flask were harvested in 1 ml of PBS containing 1× protease inhibitor cocktail by scraping. The cell suspension was subjected to 4 cycles of freezing in liquid nitrogen for 5 minutes followed by thawing at 37° C. for 5 minutes. The resulting cell lysate was centrifuged at 2000 RPM for 10 minutes to remove the cell debris and the protein concentration of the resulting 416-AD11A lysate was determined by BCA assay and aliquots frozen for future use.

### Tau-AD High Throughput Screening.

HEK293 cells stably expressing C-terminally YFP-tagged full length 0N4R-Tau carrying a P301S mutation (T24 cells) are plated at a density of 4000 cells per well of a 384-well black walled, clear bottom plate (Greiner Cat No 5678-1091Q) in 60 μl of complete DMEM (DMEM (Corning Cat No 10-013-CV), 10% FBS (VWR Cat No 97068-085, 0.5% Penicillin/streptomycin (Gibco Cat No 15140-122)) containing 0.1 μg/ml Hoechst Dye (Thermo Scientific Cat No H3570). Transfection complexes are prepared by diluting 0.15 μg of 416-AD11A lysate or HEK lysate in 5 μl of PBS per well to be transfected and 0.15 μl of Lipofectamine 2000 (LF2K) (ThermoFisher Cat No 11668500) in 5 μl of Opti-MEM per well to be transfected in separate tubes. The pre-diluted 416-AD11A or HEK lysates and LF2K are subsequently mixed and allowed to incubate for 90 minutes at RT. A 10 μl aliquot of the 416-AD11A or HEK transfection complexes is added to the appropriate wells of the 384-well plate containing the T24 cells using the Bravo liquid handling platform (Agilent). Wells A23-P24 of the 384-well plate are transfected with HEK lysate to act as a reference point for spontaneous aggregation of 0N4R^{P301S} and for transfection related toxicity. The dilution of test compounds is performed using the Bravo liquid handling platform (Agilent). The control is DMSO in the absence of any test compound. Briefly, 1 μl of each test compound (5 mM stock) in the 384-well test plate was transferred to a 384-well plate (Greiner Cat No 781280) containing 61.5 μl of complete DMEM to generate a working stock of compounds at 80 μM. An aliquot of 10 μl of these working stocks is transferred to the 384-well plate containing 60 μl of T24 cells and 10 μl of transfection complexes using the Bravo liquid handling platform resulting in a final test concentration of 10 μM. Wells A1-P2 and A23-P24 contain DMSO only. The cells are incubated for 72 h at 37° C./5% CO₂ and subsequently imaged using the Opera-Phenix Plus (Perkin-Elmer). Images are captured using the FITC (488 nm/525 nm) and DAPI (405 nm/457 nm) filter sets to image YFP-tagged 0N4R-Tau and Hoechst positive nuclei respectively. FITC images are processed using the Harmony image analysis software to determine the intensity of pixels within YFP-positive cellular aggregates as well as the number of cells that contained YFP-positive aggregates. Additionally, images captured with the DAPI filter set are processed using Harmony image analysis software to determine the number of cells per image.

| | AD Tau Aggregates |
|---|---|
| Compound | (% control) |
| 234 | 85 |
| 198 | 85 |
| 201 | 85 |
| 142 | 85 |
| 140 | 86 |
| 207 | 86 |
| 164 | 86 |
| 141 | 86 |
| 143 | 86 |
| 261 | 87 |
| 145 | 89 |
| 159 | 90 |
| 280 | 90 |
| 161 | 91 |
| 138 | 91 |
| 232 | 91 |
| 267 | 91 |
| 154 | 91 |
| 279 | 92 |
| 133 | 92 |
| 171 | 92 |
| 182 | 93 |
| 126 | 93 |
| 208 | 93 |
| 274 | 94 |
| 173 | 94 |
| 178 | 94 |
| 260 | 95 |
| 240 | 95 |
| 172 | 95 |
| 132 | 95 |
| 227 | 95 |
| 290 | 95 |
| 248 | 95 |
| 253 | 96 |
| 158 | 96 |
| 184 | 96 |
| 190 | 96 |
| 220 | 96 |
| 223 | 96 |
| 176 | 96 |
| 204 | 96 |
| 266 | 98 |
| 288 | 98 |
| 241 | 98 |
| 215 | 98 |
| 259 | 99 |
| 187 | 99 |
| 242 | 99 |
| 147 | 99 |
| 226 | 99 |
| 269 | 100 |
| 254 | 100 |
| 286 | 100 |
| 256 | 100 |
| 244 | 100 |
| 257 | 100 |
| 135 | 100 |
| 205 | 100 |
| 212 | 100 |
| 137 | 100 |
| 149 | 100 |
| 183 | 100 |
| 268 | 105 |
| 127 | 105 |
| 265 | 110 |
| 291 | 110 |
| 258 | 110 |
| 281 | 110 |
| 214 | 110 |
| 188 | 110 |
| 245 | 120 |
| 246 | 120 |
| 247 | 120 |

### REFERENCES—NUMBERS REFER TO EXAMPLE 1

1. M. E. On, A. C. Sullivan, B. Frost, A Brief Overview of Tauopathy: Causes, Consequences, and Therapeutic Strategies. Trends Pharmacol Sci 38, 637-648 (2017).
2. M. Goedert, D. S. Eisenberg, R. A. Crowther, Propagation of Tau Aggregates and Neurodegeneration. Annu Rev Neurosci 40, 189-210 (2017).
3. H. Braak, E. Braak, Neuropathological stageing of Alzheimer-related changes. Acta Neuropathol 82, 239-259 (1991).
4. P. R. Hof et al., Differential distribution of neurofibrillary tangles in the cerebral cortex of dementia pugilistica and Alzheimer's disease cases. Acta Neuropathol 85, 23-30 (1992).
5. F. E. Matthews et al., Epidemiological pathology of dementia: attributable-risks at death in the Medical Research Council Cognitive Function and Ageing Study. PLoS Med 6, e1000180 (2009).
6. K. Samgard et al., Cerebrospinal fluid total tau as a marker of Alzheimer's disease intensity. Int J Geriatr Psych 25, 403-410 (2010).
7. J. I. Ayers, B. I. Giasson, D. R. Borchelt, Prion-like Spreading in Tauopathies. Biol Psychiat 83, 337-346 (2018).
8. N. Kfoury, B. B. Holmes, H. Jiang, D. M. Holtzman, M. I. Diamond, Trans-cellular Propagation of Tau Aggregation by Fibrillar Species. J Biol Chem 287, 19440-19451 (2012).
9. D. W. Sanders et al., Distinct Tau Prion Strains Propagate in Cells and Mice and Define Different Tauopathies. Neuron 82, 1271-1288 (2014).
10. A. L. Woerman et al., Tau prions from Alzheimer's disease and chronic traumatic encephalopathy patients propagate in cultured cells. P Natl Acad Sci USA 113, E8187-E8196 (2016).
11. S. B. Prusiner, A Unifying Role for Prions in Neurodegenerative Diseases. Science 336, 1511-1513 (2012).
12. Prion Biology. (Cold Spring Harbor Laboratory Press, 2017), pp. 15.
13. A. W. P. Fitzpatrick et al., Cryo-EM structures of tau filaments from Alzheimer's disease. Nature 547, 185-190 (2017).
14. B. Falcon et al., Structures of filaments from Pick's disease reveal a novel tau protein fold. Nature 561, 137-140 (2018).
15. B. Falcon et al., Tau filaments from multiple cases of sporadic and inherited Alzheimer's disease adopt a common fold. Acta Neuropathol 136, 699-708 (2018).
16. B. Falcon et al., Novel tau filament fold in chronic traumatic encephalopathy encloses hydrophobic molecules. Nature 568, 420-423 (2019).
17. W. Zhang et al., Novel tau filament fold in corticobasal degeneration. Nature 580, 283-287 (2020).
18. Y. Shi et al., Structure-based classification of tauopathies. Nature 598, 359-363 (2021).
19. N. Okamura, K. Yanai, Florbetapir (F-18), a PET imaging agent that binds to amyloid plaques for the potential detection of Alzheimer's disease. Idrugs 13, 890-899 (2010).
20. N. Okamura, K. Yanai, Applications of tau PET imaging. Nat Rev Neurol 13, (2017).
21. C. Condello et al., Structural heterogeneity and intersubject variability of A beta in familial and sporadic Alzheimer's disease. P Natl Acad Sci USA 115, E782-E791 (2018).
22. A. M. Maxwell et al., Emergence of distinct and heterogeneous strains of amyloid beta with advanced Alzheimer's disease pathology in Down syndrome. Acta Neuropathol Com 9, (2021).
23. S. Sanabria Bohorquez et al., [(18)F]GTP1 (Genentech Tau Probe 1), a radioligand for detecting neurofibrillary tangle tau pathology in Alzheimer's disease. Eur J Nucl Med Mol Imaging 46, 2077-2089 (2019).
24. D. T. Chien et al., Early clinical PET imaging results with the novel PHF-tau radioligand [F-18]-T807. J Alzheimers Dis 34, 457-468 (2013).
25. M. Maruyama et al., Imaging of tau pathology in a tauopathy mouse model and in Alzheimer patients compared to normal controls. Neuron 79, 1094-1108 (2013).
26. C. R. Jack et al., Brain beta-amyloid measures and magnetic resonance imaging atrophy both predict time-to-progression from mild cognitive impairment to Alzheimer's disease. Brain 133, 3336-3348 (2010).
27. R. J. Bateman et al., Clinical and Biomarker Changes in Dominantly Inherited Alzheimer's Disease. New Engl J Med 367, 795-804 (2012).
28. A. Leuzy et al., Tau PET imaging in neurodegenerative tauopathies-still a challenge. Mol Psychiatry 24, 1112-1134 (2019).
29. C. Jie, V. Treyer, R. Schibli, L. Mu, Tauvid: The First FDA-Approved PET Tracer for Imaging Tau Pathology in Alzheimer's Disease. Pharmaceuticals (Basel) 14, (2021).
30. M. Honer et al., Preclinical Evaluation of F-18-RO6958948, C-11-RO6931643, and C-11-RO6924963 as Novel PET Radiotracers for Imaging Tau Aggregates in Alzheimer Disease. Journal of Nuclear Medicine 59, 675-681 (2018).
31. A. Mueller et al., Tau PET imaging with F-18-PI-2620 in Patients with Alzheimer Disease and Healthy Controls: A First-in-Humans Study. Journal of Nuclear Medicine 61, 911-919 (2020).
32. Y. Shi et al., Cryo-EM structures of tau filaments from Alzheimer's disease with PET ligand APN-1607. Acta Neuropathol 141, 697-708 (2021).
33. G. Kuang, N. A. Murugan, Y. Zhou, A. Nordberg, H. Agren, Computational Insight into the Binding Profile of the Second-Generation PET Tracer PI2620 with Tau Fibrils. ACS Chem Neurosci 11, 900-908 (2020).
34. R. Zou et al., Free Energy Profile for Penetration of Pittsburgh Compound-B into the Amyloid beta Fibril. ACS Chem Neurosci 10, 1783-1790 (2019).
35. Y. Todarwal et al., Tau Protein Binding Modes in Alzheimer's Disease for Cationic Luminescent Ligands. J Phys Chem B 125, 11628-11636 (2021).
36. B. Frieg, L. Gremer, H. Heise, D. Willbold, H. Gohlke, Binding modes of thioflavin T and Congo red to the fibril structure of amyloid-beta(1-42). Chem Commun (Camb) 56, 7589-7592 (2020).
37. D. Liebschner et al., Macromolecular structure determination using X-rays, neutrons and electrons: recent developments in Phenix. Acta Crystallogr D 75, 861-877 (2019).
38. A. Altun, F. Neese, G. Bistoni, HFLD: A Nonempirical London Dispersion-Corrected Hartree-Fock Method for the Quantification and Analysis of Noncovalent Interaction Energies of Large Molecular Systems dagger. J Chem Theory Comput 15, 5894-5907 (2019).
39. D. F. Bradley, M. K. Wolf, Aggregation of Dyes Bound to Polyanions. Proc Natl Acad Sci USA 45, 944-952 (1959).
40. G. Schwarz, Cooperative Linkages in Linear Biopolymers. H-S Z Physiol Chem 351, 131-& (1970).
41. B. H. a. B. Zimm, J. K., Theory of the Phase Transition between Helix and Random Coil in Polypeptide Chains. J Chem Phys 31, (1959).
42. A. McPherson, S. B. Larson, Investigation into the binding of dyes within protein crystals. Acta Crystallogr F 74, 593-602 (2018).
43. T. Siu et al., Discovery of a Novel cGAMP Competitive Ligand of the Inactive Form of STING. Acs Med Chem Lett 10, 92-97 (2019).
44. R. L. Wiseman et al., Flavonol Activation Defines an Unanticipated Ligand-Binding Site in the Kinase-RNase Domain of IRE1. Mol Cell 38, 291-304 (2010).
45. M. Stornaiuolo et al., Assembly of a pi-pi stack of ligands in the binding site of an acetylcholine-binding protein. Nat Commun 4, (2013).

### REFERENCES—NUMBERS REFER TO EXAMPLE 5

1 Fitzpatrick, A. W. P. et al. Cryo-EM structures of tau filaments from Alzheimer's disease. Nature 547, 185-190 (2017).
2 Schweighauser, M. et al. Structures of α-synuclein filaments from multiple system atrophy. Nature 585, 464-469 (2020).
3 Zheng, S. Q. et al. MotionCor2 anisotropic correction of beam-induced motion for improved cryo-electron microscopy. Nat Methods 14, 331-332 (2017).
4 de la Rosa-Trevin, J. M. et al. Scipion: A software framework toward integration, reproducibility and validation in 3D electron microscopy. J. Struct. Biol. 195, 93-99 (2016).
5 Kimanius, D., Dong, L., Sharov, G., Nakane, T. & Scheres, S. H. W. New tools for automated cryo-EM single-particle analysis in RELION-4.0. Biochem J 478, 4169-4185 (2021).
6 Rohou, A. & Grigorieff, N. CTFFIND4: Fast and accurate defocus estimation from electron micrographs. J Struct Biol 192, 216-221 (2015).
7 Liebschner, D. et al. Macromolecular structure determination using X-rays, neutrons and electrons: recent developments in Phenix. Acta Crystallogr D Struct Biol 75, 861-877 (2019).
8 Emsley, P., Lohkamp, B., Scott, W. G. & Cowtan, K. Features and development of Coot. Acta Crystallogr D Biol Crystallogr 66, 486-501 (2010).
9 Pettersen, E. F. et al. UCSF Chimera—a visualization system for exploratory research and analysis. J Comput Chem 25, 1605-1612 (2004).

Notwithstanding the appended claims, the disclosure set forth herein is also described by the following clauses:
1. A compound, or a salt or a hydrate or a solvate thereof, having a structure according to formula (I):

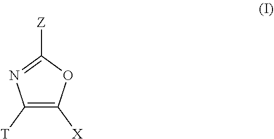

wherein
T is substituted or unsubstituted naphthyridinone or substituted or unsubstituted dihydronaphthyridinone;
X is substituted or unsubstituted aryl or substituted or unsubstituted heteroaryl, wherein X is monocyclic; and
Z is substituted or unsubstituted aryl, substituted or unsubstituted heteroaryl, substituted or unsubstituted cycloalkyl, substituted or unsubstituted heterocycloalkyl, or substituted or unsubstituted ethenyl.
2. The compound of clause 1, wherein T is

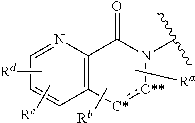

wherein R^{a}, R^{b}, R^{c}, and R^{d} are independently selected from H, halogen, substituted or unsubstituted C₁₋₃ alkyl, C₂-C₄ alkenyl, substituted or unsubstituted C₁₋₃ alkoxy, and when the connection between C* and C** is a single bond, R^{a} and R^{b} can be optionally joined with C* or with C** to form a substituted or unsubstituted cyclopropyl.
3. The method of clause 2, wherein T is

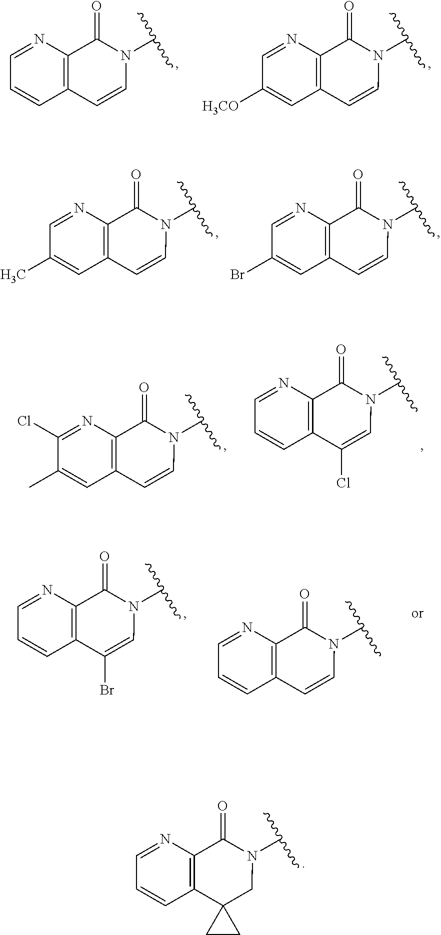

4. The compound of clause 3, wherein T is

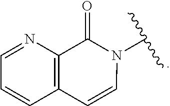 [SMILES: *n1ccc2cccnc2c1=O]

5. The compound of any of the preceding clauses, wherein X is substituted or unsubstituted phenyl, substituted or unsubstituted pyridinyl, substituted or unsubstituted pyrimidinyl, or substituted or unsubstituted thienyl.
6. The compound of any of the preceding clauses, wherein X is 4-(trifluoromethyl)phenyl, 4-fluorophenyl, 2-(trifluoromethyl)pyridin-5-yl, or 2-(1,1-difluoroethyl)pyridin-5-yl.
7. The compound of any of the preceding clauses, wherein Z is substituted or unsubstituted phenyl, substituted or unsubstituted pyridinyl, substituted or unsubstituted furan, substituted or unsubstituted thienyl, substituted or unsubstituted pyrimidinyl, or substituted or unsubstituted oxazolyl.
8. The compound of any of the preceding clauses, wherein Z is 1-methylisoquinolin-6-yl, 2-(trifluoromethyl)pyrimidin-4-yl, 2-methylpyrimidin-4-yl, 1-methyl-6-isoquinolyl, 1-(2-hydroxyethyl)-6-isoquinolyl, 3-isoquinolyl, 6-quinolyl, 8-fluoro-3-quinolyl, 8-fluoro-7-quinolyl, 4-methyl-1,7a-diaza-2-indenyl, 1-thia-5-aza-2-indenyl, 1-(2-fluoroethyl)-1H-indazol-5-yl, 3-quinolyl, 2-cyclopropyl-2H-indazol-5-yl, 6-fluoro-1,3-benzoxazol-2-yl, 5-fluoro-2-pyridyl, 1-benzofuran-2-yl, or phenyl.
9. The compound of any of the preceding clauses, having a structure according to formula (II), (III), (IV), (V), (VI), (VII), (VIII), or (IX):

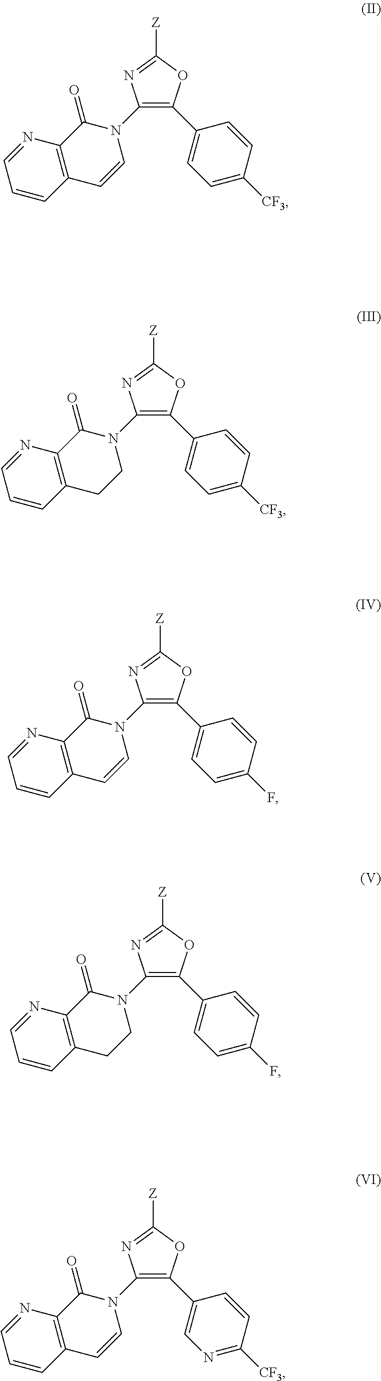

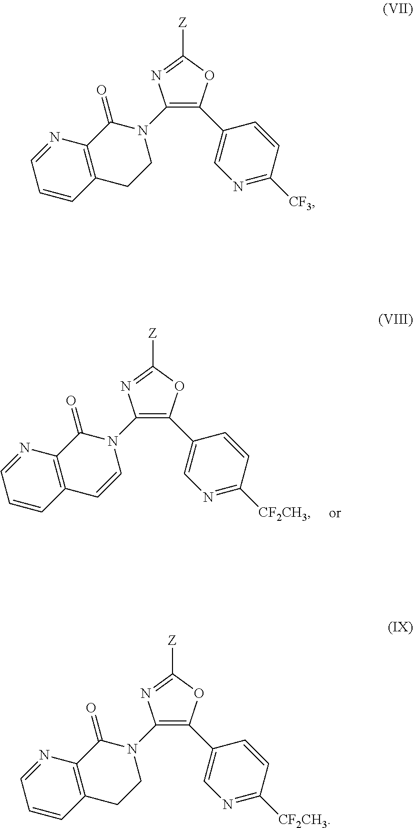

10. The compound of any of the preceding clauses, which is 7-{5-[6-(1,1-difluoroethyl)-3-pyridyl]-2-(1-methyl-6-isoquinolyl)-1,3-oxazol-4-yl}-1,7-diaza-8(7H)-naphthalenone, 7-{2-[1-(2-hydroxyethyl)-6-isoquinolyl]-5-[6-(trifluoromethyl)-3-pyridyl]-1,3-oxazol-4-yl}-1,7-diaza-8(7H)-naphthalenone, 7-{2-[1-(2-hydroxyethyl)-6-isoquinolyl]-5-[p-(trifluoromethyl)phenyl]-1,3-oxazol-4-yl}-1,7-diaza-8(7H)-naphthalenone, 7-[5-(p-fluorophenyl)-2-(3-isoquinolyl)-1,3-oxazol-4-yl]-1,7-diaza-8(7H)-naphthalenone, 7-[5-(p-fluorophenyl)-2-(6-quinolyl)-1,3-oxazol-4-yl]-1,7-diaza-8(7H)-naphthalenone, 7-{2-(8-fluoro-3-quinolyl)-5-[p-(trifluoromethyl)phenyl]-1,3-oxazol-4-yl}-1,7-diaza-8(7H)-naphthalenone, 7-[5-(p-fluorophenyl)-2-(8-fluoro-7-quinolyl)-1,3-oxazol-4-yl]-1,7-diaza-8(7H)-naphthalenone, 7-{2-(4-methyl-1,7a-diaza-2-indenyl)-5-[p-(trifluoromethyl)phenyl]-1,3-oxazol-4-yl}-1,7-diaza-8(7H)-naphthalenone, 7-{2-(1-thia-5-aza-2-indenyl)-5-[p-(trifluoromethyl)phenyl]-1,3-oxazol-4-yl}-1,7-diaza-8(7H)-naphthalenone, 7-{2-[1-(2-fluoroethyl)-1H-indazol-5-yl]-5-[p-(trifluoromethyl)phenyl]-1,3-oxazol-4-yl}-1,7-diaza-8(7H)-naphthalenone, 7-{2-(3-quinolyl)-5-[p-(trifluoromethyl)phenyl]-1,3-oxazol-4-yl}-1,7-diaza-8(7H)-naphthalenone, 7-{2-(2-cyclopropyl-2H-indazol-5-yl)-5-[p-(trifluoromethyl)phenyl]-1,3-oxazol-4-yl}-1,7-diaza-8(7H)-naphthalenone, 7-{2-(6-fluoro-1,3-benzoxazol-2-yl)-5-[p-(trifluoromethyl)phenyl]-1,3-oxazol-4-yl}-1,7-diaza-8(7H)-naphthalenone, 7-{5-[p-(trifluoromethyl)phenyl]-2-[2-(trifluoromethyl)-4-pyrimidinyl]-1,3-oxazol-4-yl}-1,7-diaza-8(7H)-naphthalenone, 7-{2-(5-fluoro-2-pyridyl)-5-[p-(trifluoromethyl)phenyl]-1,3-oxazol-4-yl}-1,7-diaza-8(7H)-naphthalenone, 7-[2-(1-benzofuran-2-yl)-5-(p-fluorophenyl)-1,3-oxazol-4-yl]-1,7-diaza-8(7H)-naphthalenone, or 7-{2-phenyl-5-[p-(trifluoromethyl)phenyl]-1,3-oxazol-4-yl}-1,7-diaza-8(7H)-naphthalenone.
11. The compound of clause 1, wherein the compound is 7-(2-(2-methylpyrimidin-4-yl)-5-(4-(trifluoromethyl)phenyl)oxazol-4-yl)-6,7-dihydro-1,7-naphthyridin-8(5H)-one.
12. The compound of clause 1, wherein the compound is N-methyl{1-[5-(3,4-difluorophenyl)-2-(4,6-dimethyl-2-pyridyl)-1,3-oxazol-4-yl]-5-fluoro-2-oxo-1,2-dihydro-4-pyrimidinyl}amine.
13. The compound of clause 1, or a salt or a hydrate or a solvate thereof, which is 7-(5-(4-(trifluoromethyl)phenyl)-2-(2-(trifluoromethyl)pyrimidin-4-yl)oxazol-4-yl)-1,7-naphthyridin-8(7H)-one.
14. The compound of clause 1, or a salt or a hydrate or a solvate thereof, which is 7-(2-(1-methylisoquinolin-6-yl)-5-(4-(trifluoromethyl)phenyl)oxazol-4-yl)-1,7-naphthyridin-8(7H)-one.
15. A method of detecting a neurological disease, comprising:
isolating brain tissue from its natural environment;
contacting the brain tissue with a labeled molecule which binds multiple sites of stacked proteins associated with a neurological disease;
determining binding of the labeled molecule; and
thereby determining a neurological disease associated with the brain tissue.
16. The method of clause 15 wherein a binding portion of the labeled molecule is characterized by:
(a) a planar core comprised of one or two rings which are optionally heterocycle;
(b) an accessible molecular conformation allowing three or more of the molecules to self-associate in a repeating, parallel-displaced stack along the stacked proteins;
(c) a configuration as shown in FIG. 3, wherein a distance between an atom within adjacent planar cores is 3.3-3.5 Å.
17. The method of any one of clauses 15-16 wherein a binding portion of the labeled molecule is further characterized by:
(d) a configuration as shown in FIG. 4, wherein a distance between equivalent atoms on two adjacent molecules is 4.8 Å.
18. The method of any one of clauses 15-17 wherein a binding portion of the labeled molecule is further characterized by:
(e) a configuration as shown in FIG. 5, wherein an angle (θ) between a line defined by two equivalent atoms on adjacent molecules and a line perpendicular to planes of the planar core is 44°.
19. The method of any one of clauses 15-18 wherein a binding portion of the labeled molecule is further characterized by:
(f) a configuration as shown in FIG. 4, wherein a minimal distance between any atom within the plane of the core and an equivalent atom within adjacent molecule bound to a stacked protein is 3.2-3.6 Å.
20. The method of any one of clauses 15-19 wherein a binding portion of the labeled molecule is further characterized by:
(g) substituents forming non-covalent interactions with the stacked proteins which interactions are selected from the group consisting of to hydrogen bonds, Van der Waals contacts, pi-pi interactions, and chalcogen bonds.
21. The method of any one of clauses 15-20 wherein the molecule is a compound according to any one of clauses 1-14.
22. The method of any one of clauses 15-20 wherein the molecule has a structure as shown in one of the structures of FIG. 6.
23. A supramolecular polymer assembly conjugate isolated from its natural environment, comprising:
stacked proteins of an α-synuclein prion associated with a neurodegenerative disease; and an α-synuclein prion inhibitor reversable bound to amyloid fibrils of the α-synuclein prion.
24. The supramolecular polymer assembly of clause 23, wherein the inhibitor is characterized by:
(a) a planar core comprised of one or two rings which are optionally heterocycle;
(b) an accessible molecular conformation allowing three or more of the molecules to self-associate in a repeating, parallel-displaced stack along the stacked proteins.
25. The supramolecular polymer assembly of any one of clauses 23 and 24, wherein the inhibitor is further characterized by:
(c) a configuration as shown in FIG. 3, wherein a distance between an atom within adjacent planar cores is 3.3-3.5 Å.
26. The supramolecular polymer assembly of any one of clauses 23-25, wherein the inhibitor is further characterized by:
(d) a configuration as shown in FIG. 4, wherein a distance between equivalent atoms on two adjacent molecules is 4.8 Å.
27. The supramolecular polymer assembly of any one of clauses 23-26, wherein the inhibitor is further characterized by:
(e) being uniquely suited to interact with the stacked proteins associated with a degenerative disease of the central nervous system (CNS).
28. The supramolecular polymer assembly of any one of clauses 23-27, wherein the inhibitor is a compound according to any one of clauses 1-14.
29. The supramolecular polymer assembly of any one of clauses 223-27, wherein the inhibitor has a structure as shown in one of the structures of FIG. 6.
30. A pharmaceutical formulation, comprising:
a pharmaceutically acceptable carrier; and
an α-synuclein prion inhibitor.
31. The pharmaceutical formulation of clause 30, wherein α-synuclein prion inhibitor is a compound according to any of clauses 1-14
32. The pharmaceutical formulation of clause 30, wherein α-synuclein prion inhibitor has a structure as shown in one of the structures of FIG. 6.
33. A method of interrupting propagation of stacked proteins associated with a neurological disease, comprising:
contacting an environment populated with stacked proteins associated with a neurological disease with molecules which bind multiple sites of the stacked proteins;
allowing the molecules to bind multiple cites of the stacked proteins; and
thereby impeding propagation of the stacked proteins in the environment.
34. The method of clause 33 wherein the molecules which binds multiple sites of the stacked proteins is characterized by:
(a) a planar core comprised of one or two rings which are optionally heterocycle;
(b) an accessible molecular conformation allowing three or more of the molecules to self-associate in a repeating, parallel-displaced stack along the stacked proteins.
35. The method of any one of clauses 33 and 34 wherein the molecules which bind multiple sites of the stacked proteins are further characterized by:
(c) a configuration as shown in FIG. 3, wherein a distance between an atom within adjacent planar cores is 3.3-3.5 Å.
36. The method of any one of clauses 33-35 wherein the molecules which bind multiple sites of the stacked proteins are further characterized by:
(d) a configuration as shown in FIG. 4, wherein a distance between equivalent atoms on two adjacent molecules is 4.8 Å.
37. The method of any one of clauses 33-36 wherein the molecules which bind multiple sites of the stacked proteins are further characterized by:
(e) a configuration as shown in FIG. 5, wherein an angle (θ) between a line defined by two equivalent atoms on adjacent molecules and a line perpendicular to planes of the planar core is 44°.
38. The method of any one of clauses 33-37 wherein the molecules which bind multiple sites of the stacked proteins are further characterized by:
(f) a configuration as shown in FIG. 3, wherein a minimal distance between any atom within the plane of the core and an equivalent atom within adjacent molecule bound to a stacked protein is 3.2-3.6 Å.
39. The method of any one of clauses 33-38 wherein the molecules which bind multiple sites of the stacked proteins are further characterized by:
(g) substituents forming non-covalent interactions with the stacked proteins which interactions are selected from the group consisting of hydrogen bonds, Van der Waals contacts, pi-pi interactions, and chalcogen bonds.
40. The method of any one of clauses 33-39 wherein the molecules which bind multiple sites of the stacked proteins is a compound according to clauses 1-14.
41. The method of any one of clauses 33-40 wherein the molecules which bind multiple sites of the stacked proteins has a structure as shown in one of the structures of FIG. 6.
42. A method of impeding progressive, templated misfolding of proteins associated with neurodegenerative disease, comprising:
administering molecules into a biological milieu containing both a propagating amyloid conformation of a protein and a native cellular form of the same protein;
allowing for formation of a complex between a supramolecular polymer assembly of the molecules and a supramolecular assembly of the protein; and
thereby impeding further sequestration of the native cellular protein and its conversion to the propagating amyloid form.
43. The method of clause 42 wherein the biological milieu is selected from the group consisting of a cell lysate, an active cell culture, and a mammalian brain.
44. The method of clause 66 wherein the mammalian brain is a human brain.
45. The method of any one of clauses 42-44 wherein the administered molecules are characterized by:
(a) a planar core comprised of one or two rings which are optionally heterocycle;
(b) an accessible molecular conformation allowing three or more of the molecules to self-associate in a repeating, parallel-displaced stack along the stacked proteins.
46. The method of any one of clauses 42-45 wherein the administered molecules are characterized by:
(c) a configuration as shown in FIG. 3, wherein a distance between an atom within adjacent planar cores is 3.3-3.5 Å.
47. The method of any one of clauses 42-46 wherein the administered molecules are characterized by:
(d) a configuration as shown in FIG. 4, wherein a distance between equivalent atoms on two adjacent molecules is 4.8 Å.
48. The method of any one of clauses 42-47 wherein the administered molecules are characterized by:
(e) a configuration as shown in FIG. 5, wherein an angle (θ) between a line defined by two equivalent atoms on adjacent molecules and a line perpendicular to planes of the planar core is 44°.
49. The method of any one of clauses 42-48 wherein the administered molecules are characterized by:
(f) a configuration as shown in FIG. 4, wherein a minimal distance between any atom within the plane of the core and an equivalent atom within adjacent molecule bound to a stacked protein is 3.2-3.6 Å.
50. The method of any one of clauses 42-49 wherein the administered molecules are characterized by:
(g) substituents forming non-covalent interactions with the stacked proteins which interactions are selected from the group consisting of hydrogen bonds, Van der Waals contacts, pi-pi interactions, and chalcogen bonds.
51. The method of any one of clauses 42-50 wherein the administered molecules are a compound according to any of clauses 1-27.
52. The method of any one of clauses 42-50 wherein the administered molecules have a structure as shown in one of the structures of FIG. 6.

Notwithstanding the appended claims, the disclosure set forth herein is also described by the following clauses:
1. A compound, or a salt or a hydrate or a solvate thereof, having a structure according to formula (I):

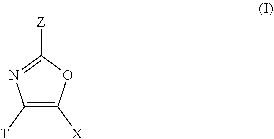

wherein T is substituted or unsubstituted naphthyridinone or substituted or unsubstituted dihydronaphthyridinone;
X is substituted or unsubstituted aryl or substituted or unsubstituted heteroaryl, wherein X is monocyclic; and
Z is substituted or unsubstituted aryl, substituted or unsubstituted heteroaryl, substituted or unsubstituted cycloalkyl, substituted or unsubstituted heterocycloalkyl, or substituted or unsubstituted ethenyl.
2. The compound of clause 1, or a salt or a hydrate or a solvate thereof, wherein T is

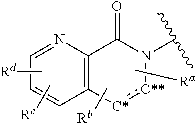

wherein R^{a}, R^{b}, R^{c}, and R^{d} are independently selected from H, halogen, substituted or unsubstituted C₁₋₃ alkyl, C₂-C₄ alkenyl, substituted or unsubstituted C₁₋₃ alkoxy, and when the connection between C* and C** is a single bond, R^{a} and R^{b} can be optionally joined with C* or with C** to form a substituted or unsubstituted cyclopropyl.
3. The compound of clause 1, or a salt or a hydrate or a solvate thereof, wherein T is

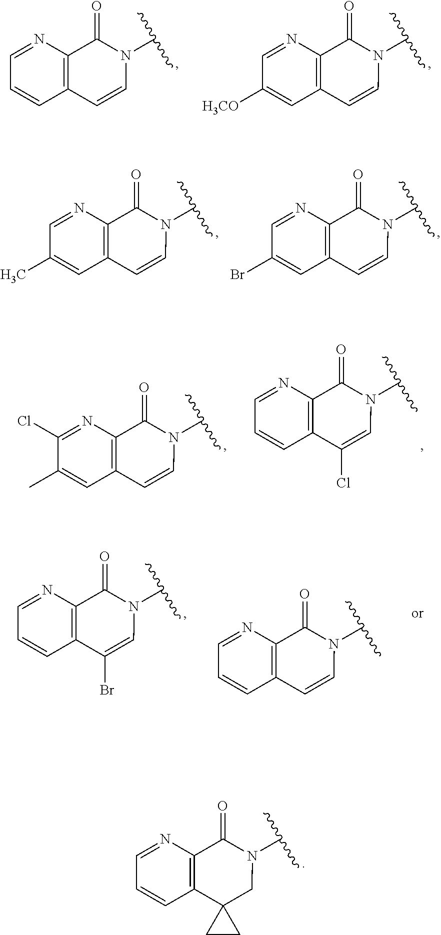

4. The compound of clause 1, or a salt or a hydrate or a solvate thereof, wherein T is

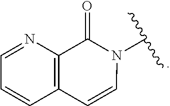 [SMILES: *n1ccc2cccnc2c1=O]

5. The compound of a preceding clause, or a salt or a hydrate or a solvate thereof, wherein X is substituted or unsubstituted phenyl, substituted or unsubstituted pyridinyl, substituted or unsubstituted pyrimidinyl, or substituted or unsubstituted thienyl.
6. The compound of a preceding clause, or a salt or a hydrate or a solvate thereof, wherein X is 4-(trifluoromethyl)phenyl, 4-fluorophenyl, 2-(trifluoromethyl)pyridin-5-yl, or 2-(1,1-difluoroethyl)pyridin-5-yl.
7. The compound of a preceding clause, or a salt or a hydrate or a solvate thereof, wherein Z is substituted or unsubstituted phenyl, substituted or unsubstituted pyridinyl, substituted or unsubstituted furan, substituted or unsubstituted thienyl, substituted or unsubstituted pyrimidinyl, or substituted or unsubstituted oxazolyl.
8. The compound of a preceding clause, or a salt or a hydrate or a solvate thereof, wherein Z is 1-methylisoquinolin-6-yl, 2-(trifluoromethyl)pyrimidin-4-yl, 2-methylpyrimidin-4-yl, 1-methyl-6-isoquinolyl, 1-(2-hydroxyethyl)-6-isoquinolyl, 3-isoquinolyl, 6-quinolyl, 8-fluoro-3-quinolyl, 8-fluoro-7-quinolyl, 4-methyl-1,7a-diaza-2-indenyl, 1-thia-5-aza-2-indenyl, 1-(2-fluoroethyl)-1H-indazol-5-yl, 3-quinolyl, 2-cyclopropyl-2H-indazol-5-yl, 6-fluoro-1,3-benzoxazol-2-yl, 5-fluoro-2-pyridyl, 1-benzofuran-2-yl, or phenyl.
9. The compound of a preceding clause, or a salt or a hydrate or a solvate thereof, having a structure according to formula (II), (III), (IV), (V), (VI), (VII), (VIII), or (IX):

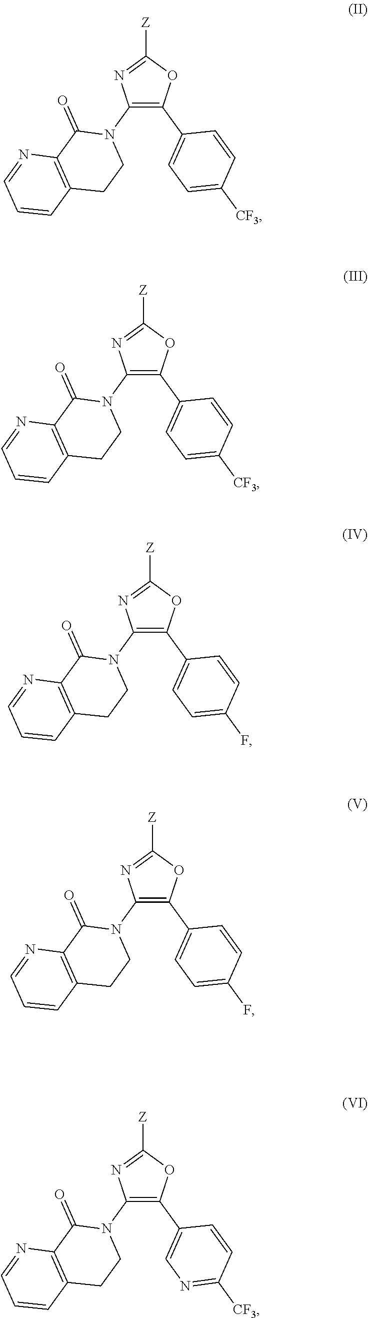

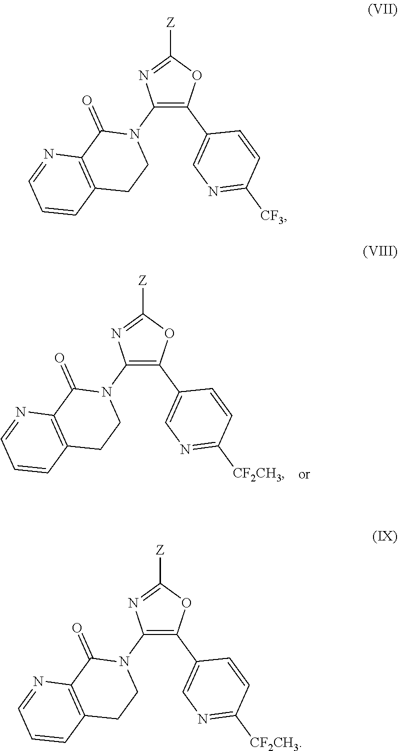

10. The compound of clause 1, or a salt or a hydrate or a solvate thereof, which is 7-{5-[6-(1,1-difluoroethyl)-3-pyridyl]-2-(1-methyl-6-isoquinolyl)-1,3-oxazol-4-yl}-1,7-diaza-8(7H)-naphthalenone, 7-{2-[1-(2-hydroxyethyl)-6-isoquinolyl]-5-[6-(trifluoromethyl)-3-pyridyl]-1,3-oxazol-4-yl}-1,7-diaza-8(7H)-naphthalenone, 7-{2-[1-(2-hydroxyethyl)-6-isoquinolyl]-5-[p-(trifluoromethyl)phenyl]-1,3-oxazol-4-yl}-1,7-diaza-8(7H)-naphthalenone, 7-[5-(p-fluorophenyl)-2-(3-isoquinolyl)-1,3-oxazol-4-yl]-1,7-diaza-8(7H)-naphthalenone, 7-[5-(p-fluorophenyl)-2-(6-quinolyl)-1,3-oxazol-4-yl]-1,7-diaza-8(7H)-naphthalenone, 7-{2-(8-fluoro-3-quinolyl)-5-[p-(trifluoromethyl)phenyl]-1,3-oxazol-4-yl}-1,7-diaza-8(7H)-naphthalenone, 7-[5-(p-fluorophenyl)-2-(8-fluoro-7-quinolyl)-1,3-oxazol-4-yl]-1,7-diaza-8(7H)-naphthalenone, 7-{2-(4-methyl-1,7a-diaza-2-indenyl)-5-[p-(trifluoromethyl)phenyl]-1,3-oxazol-4-yl}-1,7-diaza-8(7H)-naphthalenone, 7-{2-(1-thia-5-aza-2-indenyl)-5-[p-(trifluoromethyl)phenyl]-1,3-oxazol-4-yl}-1,7-diaza-8(7H)-naphthalenone, 7-{2-[1-(2-fluoroethyl)-1H-indazol-5-yl]-5-[p-(trifluoromethyl)phenyl]-1,3-oxazol-4-yl}-1,7-diaza-8(7H)-naphthalenone, 7-{2-(3-quinolyl)-5-[p-(trifluoromethyl)phenyl]-1,3-oxazol-4-yl}-1,7-diaza-8(7H)-naphthalenone, 7-{2-(2-cyclopropyl-2H-indazol-5-yl)-5-[p-(trifluoromethyl)phenyl]-1,3-oxazol-4-yl}-1,7-diaza-8(7H)-naphthalenone, 7-{2-(6-fluoro-1,3-benzoxazol-2-yl)-5-[p-(trifluoromethyl)phenyl]-1,3-oxazol-4-yl}-1,7-diaza-8(7H)-naphthalenone, 7-{5-[p-(trifluoromethyl)phenyl]-2-[2-(trifluoromethyl)-4-pyrimidinyl]-1,3-oxazol-4-yl}-1,7-diaza-8(7H)-naphthalenone, 7-{2-(5-fluoro-2-pyridyl)-5-[p-(trifluoromethyl)phenyl]-1,3-oxazol-4-yl}-1,7-diaza-8(7H)-naphthalenone, 7-{2-(1-benzofuran-2-yl)-5-(p-fluorophenyl)-1,3-oxazol-4-yl}-1,7-diaza-8(7H)-naphthalenone, or 7-{2-phenyl-5-[p-(trifluoromethyl)phenyl]-1,3-oxazol-4-yl}-1,7-diaza-8(7H)-naphthalenone.
11. The compound of clause 1, or a salt or a hydrate or a solvate thereof, which is 7-(2-(2-methylpyrimidin-4-yl)-5-(4-(trifluoromethyl)phenyl)oxazol-4-yl)-6,7-dihydro-1,7-naphthyridin-8(5H)-one.
12. The compound of clause 1, or a salt or a hydrate or a solvate thereof, which is 7-(5-(4-(trifluoromethyl)phenyl)-2-(2-(trifluoromethyl)pyrimidin-4-yl)oxazol-4-yl)-1,7-naphthyridin-8(7H)-one.
13. The compound of clause 1, or a salt or a hydrate or a solvate thereof, which is 7-(2-(1-methylisoquinolin-6-yl)-5-(4-(trifluoromethyl)phenyl)oxazol-4-yl)-1,7-naphthyridin-8(7H)-one.
14. A compound of formula (XVI):

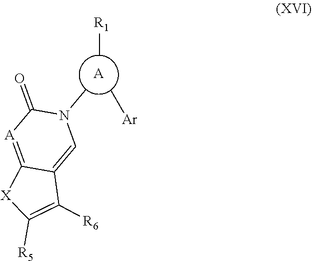

wherein:
   R₁, R₅, and R₆ are each independently selected from aryl, substituted aryl, heteroaryl, substituted heteroaryl, alkyl, substituted alkyl, —CH₂R₂₀, —CHMeR₂₀, —CH(OH)R₂₀, cycloalkyl, substituted cycloalkyl, heterocycle, substituted heterocycle, alkenyl, alkynyl, acyl, nitro, halo, amino, substituted amine, ether, thioether, and H;
   Ar is selected from aryl, substituted aryl, heteroaryl, and substituted heteroaryl;
   A is selected from N, CH, C(halo);
   X is selected from O and N—R₂,
R₂ and R₂₀ are each independently selected from aryl, substituted aryl, heteroaryl, substituted heteroaryl, alkyl, substituted alkyl, cycloalkyl, substituted cycloalkyl, heterocycle, substituted heterocycle, alkenyl, alkynyl, acyl, and H; and
Ring B is a 5-membered heteroaryl ring.
15. The compound of clause 14, wherein the compound has formula (XVII):

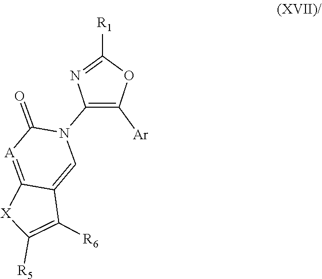

16. The compound of clause 15, wherein the compound has formula (III):

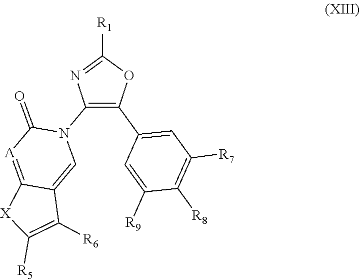

wherein:
R₇₋₉ each are independently selected from halogen, H, alkyl, substituted alkyl, alkoxy, substituted alkoxyl.
17. The compound of clause 16, wherein the compound has formula (XIII)(a), (XIII)(b), (XIII)(c), (XIII)(d), (XIII)(e), or (XIII)(f):

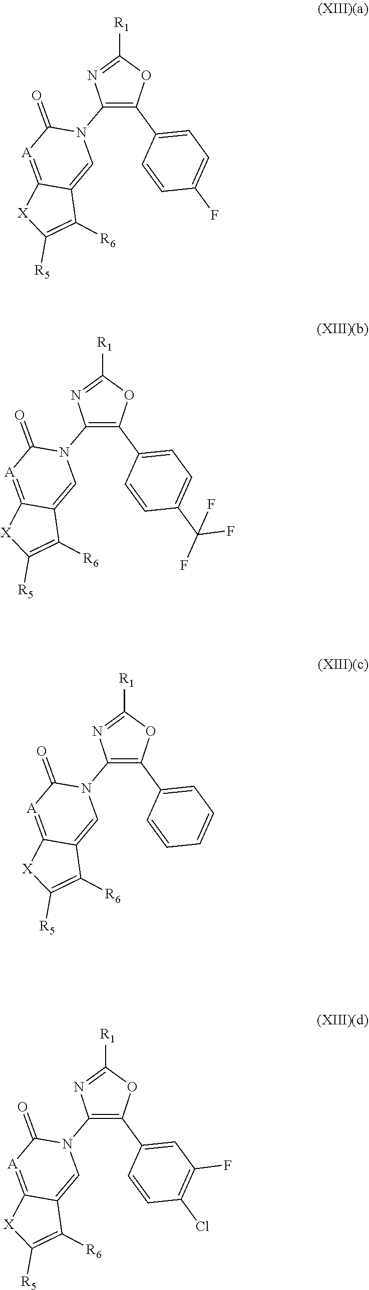

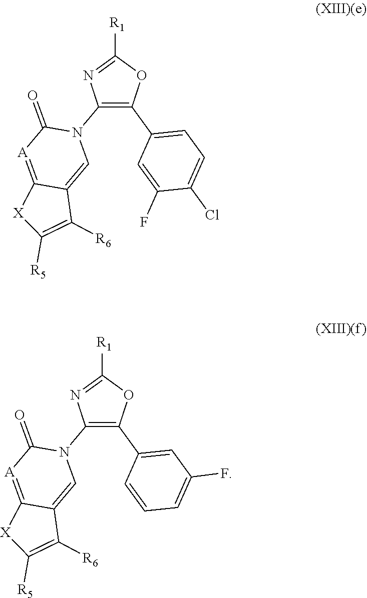

18. The compound of clause 16, wherein the compound has formula (XIV)(a) or (XIV)(b):

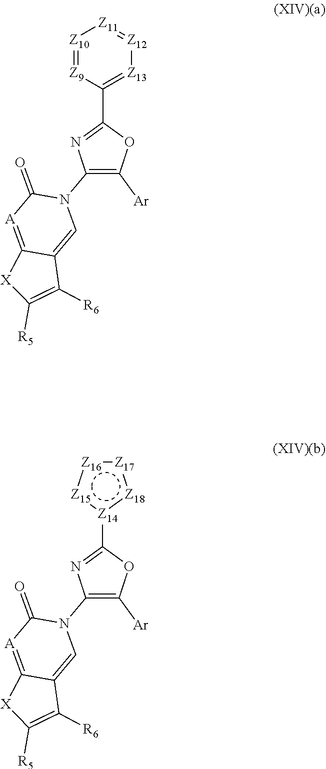

wherein:
Z₉-Z₁₃ are each independently selected from N and CR_{Z};
Z₁₄-Z₁₈ are each independently selected from N, O, S, and CR_{Z};
each R_{Z} is independently selected from H, alkyl, substituted alkyl, cycloalkyl, substituted alkyl, heterocycloalkyl, substituted heterocycloalkyl.
20. A pharmaceutical formulation comprising:
a) the compound, or a pharmaceutically acceptable salt or a hydrate or a solvate thereof, of any of clauses 1-18; and
b) a pharmaceutically acceptable excipient.

Notwithstanding the appended claims, the disclosure set forth herein is also described by the following clauses:

1. A method of detecting a neurological disease, comprising:
contacting the brain tissue with a labeled molecule which binds multiple sites of stacked proteins associated with a neurological disease;
determining binding of the labeled molecule; and
thereby determining a neurological disease associated with the brain tissue.

2. The method of clause 1 wherein the determining comprises performing positron emission tomography, imaging mass spectrometry, or magnetic resonance imaging.

3. The method of clause 1 or 2 wherein a binding portion of the labeled molecule is characterized by:
(a) a planar core comprised of one or two rings which are optionally heterocycle;
(b) an accessible molecular conformation allowing three or more of the molecules to self-associate in a repeating, parallel-displaced stack along the stacked proteins;
(c) a configuration as shown in FIG. 3, wherein a distance between an atom within adjacent planar cores is 3.3-3.5 Å; and
(d) a configuration as shown in FIG. 4, wherein a distance between equivalent atoms on two adjacent molecules is 4.8 Å.

4. The method of any one of clauses 1-3 wherein a binding portion of the labeled molecule is further characterized by at least one of:
(e) a configuration as shown in FIG. 5, wherein an angle (θ) between a line defined by two equivalent atoms on adjacent molecules and a line perpendicular to planes of the planar core is 44′;
(f) a configuration as shown in FIG. 4, wherein a minimal distance between any atom within the plane of the core and an equivalent atom within adjacent molecule bound to a stacked protein is 3.2-3.6 Å; and
(g) substituents forming non-covalent interactions with the stacked proteins which interactions are selected from the group consisting of to hydrogen bonds, Van der Waals contacts, pi-pi interactions, and chalcogen bonds.

5. The method of any of clauses 1-4, wherein the labeled molecule contains one or more of [2H], [3H], [11C], [18F], or [13N].

6. The method of clauses 1-5, wherein the stacked proteins are selected from the group consisting of α-synuclein, tau, and amyloid β.

7. The method of clause 1, wherein the labeled molecule is a compound, or a salt or a hydrate or a solvate thereof, having a structure according to formula (I):

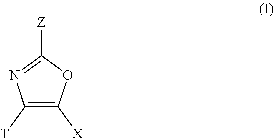

wherein
T is substituted or unsubstituted naphthyridinone or substituted or unsubstituted dihydronaphthyridinone;
X is substituted or unsubstituted aryl or substituted or unsubstituted heteroaryl, wherein X is monocyclic; and
Z is substituted or unsubstituted aryl, substituted or unsubstituted heteroaryl, substituted or unsubstituted cycloalkyl, substituted or unsubstituted heterocycloalkyl, or substituted or unsubstituted ethenyl.

8. The method of clause 7, wherein T is

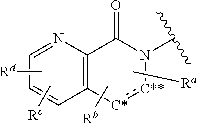

wherein R^{a}, R^{b}, R^{c}, and R^{d} are independently selected from H, halogen, substituted or unsubstituted C₁₋₃ alkyl, C₂-C₄ alkenyl, substituted or unsubstituted C₁₋₃ alkoxy, and when the connection between C* and C** is a single bond, R^{a} and R^{b} can be optionally joined with C* or with C** to form a substituted or unsubstituted cyclopropyl.

9. The method of clause 7, wherein T is

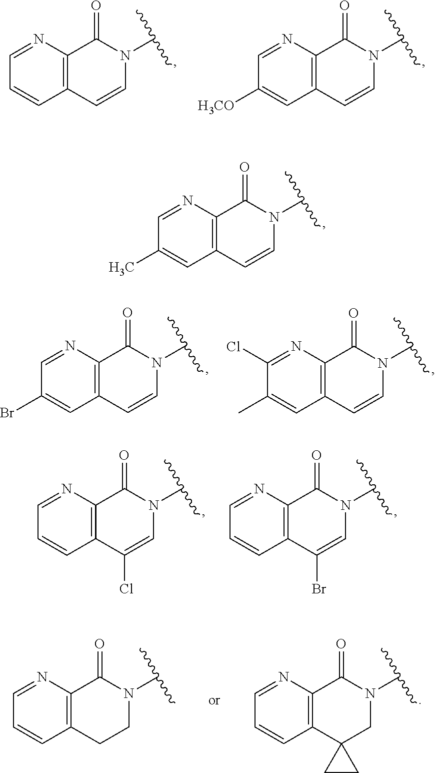

10. The method of clause 7, wherein T is

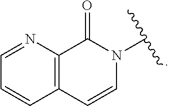 [SMILES: *n1ccc2cccnc2c1=O]

11. The method of any of clauses 7-10, wherein X is substituted or unsubstituted phenyl, substituted or unsubstituted pyridinyl, substituted or unsubstituted pyrimidinyl, or substituted or unsubstituted thienyl.

12. The method of any of clauses 7-11, wherein X is 4-(trifluoromethyl)phenyl, 4-fluorophenyl, 2-(trifluoromethyl)pyridin-5-yl, or 2-(1,1-difluoroethyl)pyridin-5-yl.

13. The method of any of clauses 7-12, wherein Z is substituted or unsubstituted phenyl, substituted or unsubstituted pyridinyl, substituted or unsubstituted furan, substituted or unsubstituted thienyl, substituted or unsubstituted pyrimidinyl, or substituted or unsubstituted oxazolyl.

14. The method of any of clauses 7-13, wherein Z is 1-methylisoquinolin-6-yl, 2-(trifluoromethyl)pyrimidin-4-yl, 2-methylpyrimidin-4-yl, 1-methyl-6-isoquinolyl, 1-(2-hydroxyethyl)-6-isoquinolyl, 3-isoquinolyl, 6-quinolyl, 8-fluoro-3-quinolyl, 8-fluoro-7-quinolyl, 4-methyl-1,7a-diaza-2-indenyl, 1-thia-5-aza-2-indenyl, 1-(2-fluoroethyl)-1H-indazol-5-yl, 3-quinolyl, 2-cyclopropyl-2H-indazol-5-yl, 6-fluoro-1,3-benzoxazol-2-yl, 5-fluoro-2-pyridyl, 1-benzofuran-2-yl, or phenyl.

15. The method of any of clauses 7-14, having a structure according to formula (II), (III), (IV), (V), (VI), (VII), (VIII), or (IX):

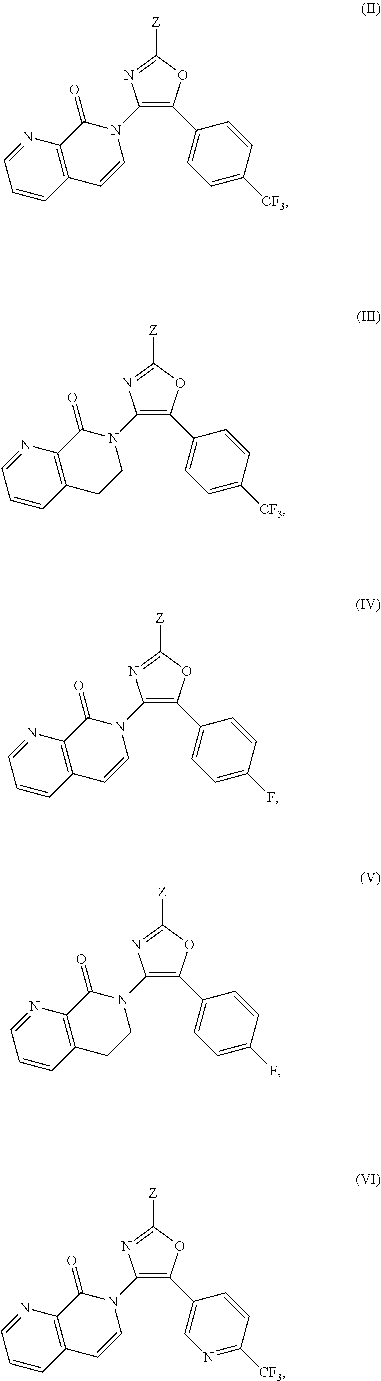

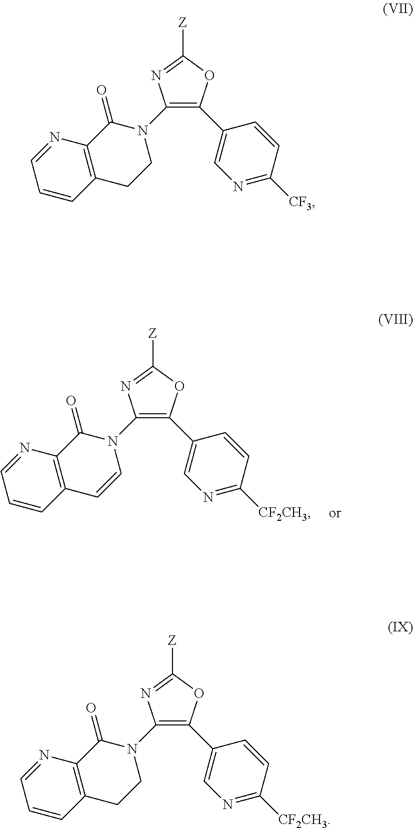

16. The method of any of clauses 7-15, which is 7-{5-[6-(1,1-difluoroethyl)-3-pyridyl]-2-(1-methyl-6-isoquinolyl)-1,3-oxazol-4-yl}-1,7-diaza-8(7H)-naphthalenone, 7-{2-[1-(2-hydroxyethyl)-6-isoquinolyl]-5-[6-(trifluoromethyl)-3-pyridyl]-1,3-oxazol-4-yl}-1,7-diaza-8(7H)-naphthalenone, 7-{2-[1-(2-hydroxyethyl)-6-isoquinolyl]-5-[p-(trifluoromethyl)phenyl]-1,3-oxazol-4-yl}-1,7-diaza-8(7H)-naphthalenone, 7-[5-(p-fluorophenyl)-2-(3-isoquinolyl)-1,3-oxazol-4-yl]-1,7-diaza-8(7H)-naphthalenone, 7-[5-(p-fluorophenyl)-2-(6-quinolyl)-1,3-oxazol-4-yl]-1,7-diaza-8(7H)-naphthalenone, 7-{2-(8-fluoro-3-quinolyl)-5-[p-(trifluoromethyl)phenyl]-1,3-oxazol-4-yl}-1,7-diaza-8(7H)-naphthalenone, 7-[5-(p-fluorophenyl)-2-(8-fluoro-7-quinolyl)-1,3-oxazol-4-yl]-1,7-diaza-8(7H)-naphthalenone, 7-{2-(4-methyl-1,7a-diaza-2-indenyl)-5-[p-(trifluoromethyl)phenyl]-1,3-oxazol-4-yl}-1,7-diaza-8(7H)-naphthalenone, 7-{2-(1-thia-5-aza-2-indenyl)-5-[p-(trifluoromethyl)phenyl]-1,3-oxazol-4-yl}-1,7-diaza-8(7H)-naphthalenone, 7-{2-[1-(2-fluoroethyl)-1H-indazol-5-yl]-5-[p-(trifluoromethyl)phenyl]-1,3-oxazol-4-yl}-1,7-diaza-8(7H)-naphthalenone, 7-{2-(3-quinolyl)-5-[p-(trifluoromethyl)phenyl]-1,3-oxazol-4-yl}-1,7-diaza-8(7H)-naphthalenone, 7-{2-(2-cyclopropyl-2H-indazol-5-yl)-5-[p-(trifluoromethyl)phenyl]-1,3-oxazol-4-yl}-1,7-diaza-8(7H)-naphthalenone, 7-{2-(6-fluoro-1,3-benzoxazol-2-yl)-5-[p-(trifluoromethyl)phenyl]-1,3-oxazol-4-yl}-1,7-diaza-8(7H)-naphthalenone, 7-{5-[p-(trifluoromethyl)phenyl]-2-[2-(trifluoromethyl)-4-pyrimidinyl]-1,3-oxazol-4-yl}-1,7-diaza-8(7H)-naphthalenone, 7-{2-(5-fluoro-2-pyridyl)-5-[p-(trifluoromethyl)phenyl]-1,3-oxazol-4-yl}-1,7-diaza-8(7H)-naphthalenone, 7-[2-(1-benzofuran-2-yl)-5-(p-fluorophenyl)-1,3-oxazol-4-yl]-1,7-diaza-8(7H)-naphthalenone, or 7-{2-phenyl-5-[p-(trifluoromethyl)phenyl]-1,3-oxazol-4-yl}-1,7-diaza-8(7H)-naphthalenone.

17. The method of clause 7, wherein the compound is 7-(2-(2-methylpyrimidin-4-yl)-5-(4-(trifluoromethyl)phenyl)oxazol-4-yl)-6,7-dihydro-1,7-naphthyridin-8(5H)-one.

18. The method of clause 7, wherein the compound is N-methyl {1-[5-(3,4-difluorophenyl)-2-(4,6-dimethyl-2-pyridyl)-1,3-oxazol-4-yl]-5-fluoro-2-oxo-1,2-dihydro-4-pyrimidinyl}amine.

19. The method of clause 1, wherein the labeled molecule is a compound of formula (XVI):

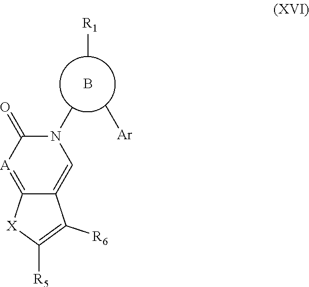

wherein:
R₁, R₅, and R₆ are each independently selected from aryl, substituted aryl, heteroaryl, substituted heteroaryl, alkyl, substituted alkyl, —CH₂R₂₀, —CHMeR₂₀, —CH(OH)R₂₀, cycloalkyl, substituted cycloalkyl, heterocycle, substituted heterocycle, alkenyl, alkynyl, acyl, nitro, halo, amino, substituted amine, ether, thioether, and H;
Ar is selected from aryl, substituted aryl, heteroaryl, and substituted heteroaryl;
A is selected from N, CH, C(halo);
X is selected from O and N—R₂,
R₂ and R₂₀ are each independently selected from aryl, substituted aryl, heteroaryl, substituted heteroaryl, alkyl, substituted alkyl, cycloalkyl, substituted cycloalkyl, heterocycle, substituted heterocycle, alkenyl, alkynyl, acyl, and H; and Ring B is a 5-membered heteroaryl ring.

20. The method of clause 1, wherein the molecule has a structure as shown in one of the structures of FIG. 6.

21. The method of any one of clauses 1-6, wherein the determining comprises positron emission tomography.

22. The method of any one of clauses 1-6, wherein the determining comprises imaging mass spectrometry.

23. The method of any one of clauses 1-6, wherein the determining comprises magnetic resonance imaging.

24. The method of any of the preceding clauses, wherein the neurological disease is selected from the group consisting of multiple systems atrophy, Parkinson's disease, and Alzheimer's disease.

Notwithstanding the appended claims, the disclosure set forth herein is also described by the following clauses:

1. A method of interrupting propagation of stacked proteins associated with a neurological disease, comprising:
contacting an environment populated with stacked proteins associated with a neurological disease with molecules which bind multiple sites of the stacked proteins;
allowing the molecules to bind multiple sites of the stacked proteins; and
thereby impeding propagation of the stacked proteins in the environment.

2. The method of clause 1 wherein the molecules which binds multiple sites of the stacked proteins is characterized by:
(a) a planar core comprised of one or two rings which are optionally heterocycle;
(b) an accessible molecular conformation allowing three or more of the molecules to self-associate in a repeating, parallel-displaced stack along the stacked proteins;
(c) a configuration as shown in FIG. 3, wherein a distance between an atom within adjacent planar cores is 3.3-3.5 Å; and
(d) a configuration as shown in FIG. 4, wherein a distance between equivalent atoms on two adjacent molecules is 4.8 Å.

3. The method of any one of clauses 1-2 wherein the molecules which bind multiple sites of the stacked proteins are further characterized by at least one of:
(e) a configuration as shown in FIG. 5, wherein an angle (θ) between a line defined by two equivalent atoms on adjacent molecules and a line perpendicular to planes of the planar core is 44′;
(f) a configuration as shown in FIG. 3, wherein a minimal distance between any atom within the plane of the core and an equivalent atom within adjacent molecule bound to a stacked protein is 3.2-3.6 Å; and
(g) substituents forming non-covalent interactions with the stacked proteins which interactions are selected from the group consisting of hydrogen bonds, Van der Waals contacts, pi-pi interactions, and chalcogen bonds.

4. The method of any of clauses 1-3, wherein the stacked proteins are selected from the group consisting of tau, α-synuclein, and amyloid b.

5. The method of any of clauses 1-4, wherein the contacting comprises administering to an individual who has or is suspected to have the neurological disease an effective amount of the molecule.

6. The method of clause 5, wherein the impeding propagation of the stacked proteins in the environment treats the neurological disease.

7. The method of clause 1, wherein the molecule is a compound, or a salt or a hydrate or a solvate thereof, having a structure according to formula (I):

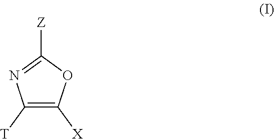

wherein
T is substituted or unsubstituted naphthyridinone or substituted or unsubstituted dihydronaphthyridinone;
X is substituted or unsubstituted aryl or substituted or unsubstituted heteroaryl, wherein X is monocyclic; and
Z is substituted or unsubstituted aryl, substituted or unsubstituted heteroaryl, substituted or unsubstituted cycloalkyl, substituted or unsubstituted heterocycloalkyl, or substituted or unsubstituted ethenyl.

8. The method of clause 7, wherein T is

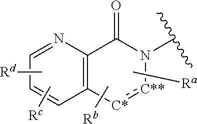

wherein R^{a}, R^{b}, R^{c}, and R^{d} are independently selected from H, halogen, substituted or unsubstituted C₁₋₃ alkyl, C₂-C₄ alkenyl, substituted or unsubstituted C₁₋₃ alkoxy, and when the connection between C* and C** is a single bond, R^{a} and R^{b} can be optionally joined with C* or with C** to form a substituted or unsubstituted cyclopropyl.

9. The method of clause 7, wherein T is

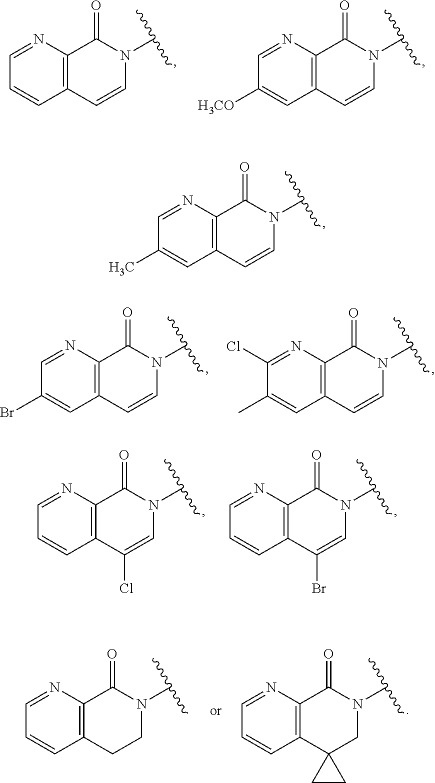

10. The method of clause 7, wherein T is

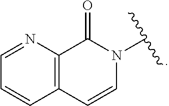 [SMILES: *n1ccc2cccnc2c1=O]

11. The method of any of clauses 7-10, wherein X is substituted or unsubstituted phenyl, substituted or unsubstituted pyridinyl, substituted or unsubstituted pyrimidinyl, or substituted or unsubstituted thienyl.

12. The method of any of clauses 7-11, wherein X is 4-(trifluoromethyl)phenyl, 4-fluorophenyl, 2-(trifluoromethyl)pyridin-5-yl, or 2-(1,1-difluoroethyl)pyridin-5-yl.

13. The method of any of clauses 7-12, wherein Z is substituted or unsubstituted phenyl, substituted or unsubstituted pyridinyl, substituted or unsubstituted furan, substituted or unsubstituted thienyl, substituted or unsubstituted pyrimidinyl, or substituted or unsubstituted oxazolyl.

14. The method of any of clauses 7-13, wherein Z is 1-methylisoquinolin-6-yl, 2-(trifluoromethyl)pyrimidin-4-yl, 2-methylpyrimidin-4-yl, 1-methyl-6-isoquinolyl, 1-(2-hydroxyethyl)-6-isoquinolyl, 3-isoquinolyl, 6-quinolyl, 8-fluoro-3-quinolyl, 8-fluoro-7-quinolyl, 4-methyl-1,7a-diaza-2-indenyl, 1-thia-5-aza-2-indenyl, 1-(2-fluoroethyl)-1H-indazol-5-yl, 3-quinolyl, 2-cyclopropyl-2H-indazol-5-yl, 6-fluoro-1,3-benzoxazol-2-yl, 5-fluoro-2-pyridyl, 1-benzofuran-2-yl, or phenyl.

15. The method of any of clauses 7-14, having a structure according to formula (II), (III), (IV), (V), (VI), (VII), (VIII), or (IX):

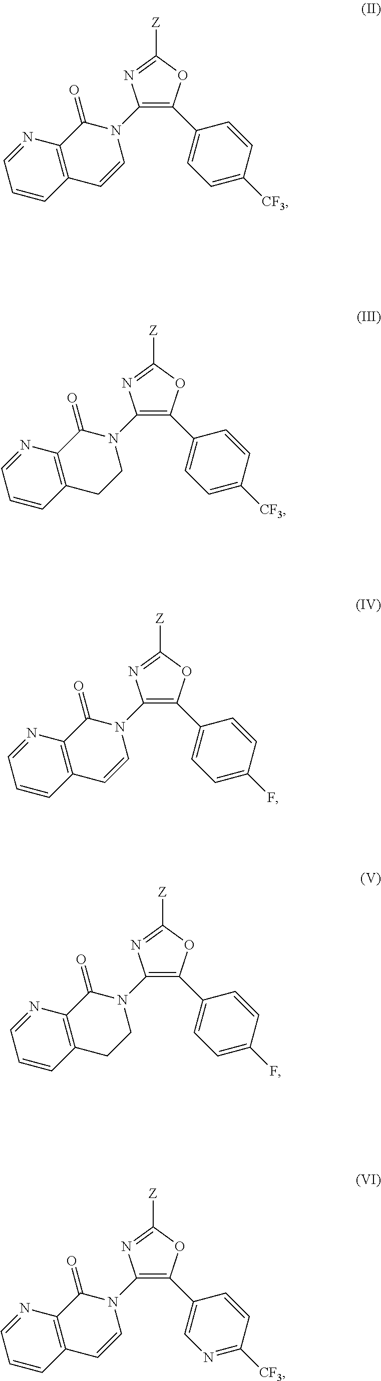

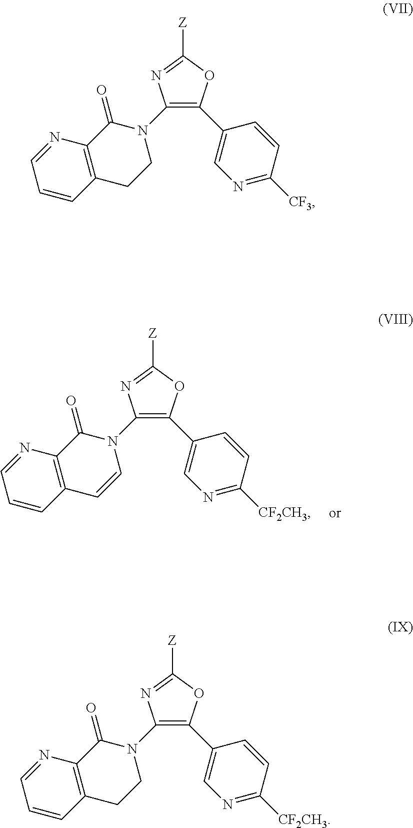

16. The method of any of clauses 7-15, which is 7-{5-[6-(1,1-difluoroethyl)-3-pyridyl]-2-(1-methyl-6-isoquinolyl)-1,3-oxazol-4-yl}-1,7-diaza-8(7H)-naphthalenone, 7-{2-[1-(2-hydroxyethyl)-6-isoquinolyl]-5-[6-(trifluoromethyl)-3-pyridyl]-1,3-oxazol-4-yl}-1,7-diaza-8(7H)-naphthalenone, 7-{2-[1-(2-hydroxyethyl)-6-isoquinolyl]-5-[p-(trifluoromethyl)phenyl]-1,3-oxazol-4-yl}-1,7-diaza-8(7H)-naphthalenone, 7-[5-(p-fluorophenyl)-2-(3-isoquinolyl)-1,3-oxazol-4-yl]-1,7-diaza-8(7H)-naphthalenone, 7-[5-(p-fluorophenyl)-2-(6-quinolyl)-1,3-oxazol-4-yl]-1,7-diaza-8(7H)-naphthalenone, 7-{2-(8-fluoro-3-quinolyl)-5-[p-(trifluoromethyl)phenyl]-1,3-oxazol-4-yl}-1,7-diaza-8(7H)-naphthalenone, 7-[5-(p-fluorophenyl)-2-(8-fluoro-7-quinolyl)-1,3-oxazol-4-yl]-1,7-diaza-8(7H)-naphthalenone, 7-{2-(4-methyl-1,7a-diaza-2-indenyl)-5-[p-(trifluoromethyl)phenyl]-1,3-oxazol-4-yl}-1,7-diaza-8(7H)-naphthalenone, 7-{2-(1-thia-5-aza-2-indenyl)-5-[p-(trifluoromethyl)phenyl]-1,3-oxazol-4-yl}-1,7-diaza-8(7H)-naphthalenone, 7-{2-[1-(2-fluoroethyl)-1H-indazol-5-yl]-5-[p-(trifluoromethyl)phenyl]-1,3-oxazol-4-yl}-1,7-diaza-8(7H)-naphthalenone, 7-{2-(3-quinolyl)-5-[p-(trifluoromethyl)phenyl]-1,3-oxazol-4-yl}-1,7-diaza-8(7H)-naphthalenone, 7-{2-(2-cyclopropyl-2H-indazol-5-yl)-5-[p-(trifluoromethyl)phenyl]-1,3-oxazol-4-yl}-1,7-diaza-8(7H)-naphthalenone, 7-{2-(6-fluoro-1,3-benzoxazol-2-yl)-5-[p-(trifluoromethyl)phenyl]-1,3-oxazol-4-yl}-1,7-diaza-8(7H)-naphthalenone, 7-{5-[p-(trifluoromethyl)phenyl]-2-[2-(trifluoromethyl)-4-pyrimidinyl]-1,3-oxazol-4-yl}-1,7-diaza-8(7H)-naphthalenone, 7-{2-(5-fluoro-2-pyridyl)-5-[p-(trifluoromethyl)phenyl]-1,3-oxazol-4-yl}-1,7-diaza-8(7H)-naphthalenone, 7-{2-(1-benzofuran-2-yl)-5-(p-fluorophenyl)-1,3-oxazol-4-yl}-1,7-diaza-8(7H)-naphthalenone, or 7-{2-phenyl-5-[p-(trifluoromethyl)phenyl]-1,3-oxazol-4-yl}-1,7-diaza-8(7H)-naphthalenone.

17. The method of clause 7, wherein the compound is 7-(5-(4-(trifluoromethyl)phenyl)-2-(2-(trifluoromethyl)pyrimidin-4-yl)oxazol-4-yl)-1,7-naphthyridin-8(7H)-one.

18. The method of clause 7, wherein the compound is 7-(2-(1-methylisoquinolin-6-yl)-5-(4-(trifluoromethyl)phenyl)oxazol-4-yl)-1,7-naphthyridin-8(7H)-one.

19. The method of clause 1, wherein the labeled molecule is a compound of formula (XVI):

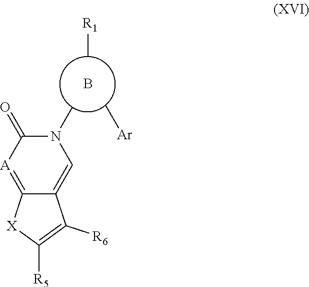

wherein:
R₁, R₅, and R₆ are each independently selected from aryl, substituted aryl, heteroaryl, substituted heteroaryl, alkyl, substituted alkyl, —CH₂R₂₀, —CHMeR₂₀, —CH(OH)R₂₀, cycloalkyl, substituted cycloalkyl, heterocycle, substituted heterocycle, alkenyl, alkynyl, acyl, nitro, halo, amino, substituted amine, ether, thioether, and H;
Ar is selected from aryl, substituted aryl, heteroaryl, and substituted heteroaryl;
A is selected from N, CH, C(halo);
X is selected from O and N—R₂,
R₂ and R₂₀ are each independently selected from aryl, substituted aryl, heteroaryl, substituted heteroaryl, alkyl, substituted alkyl, cycloalkyl, substituted cycloalkyl, heterocycle, substituted heterocycle, alkenyl, alkynyl, acyl, and H; and
Ring B is a 5-membered heteroaryl ring.

20. The method of any one of clauses 1-6, wherein the molecule has a structure as shown in one of the structures of FIG. 6.

21. The method of any of the preceding clauses, wherein the neurological disease is selected from the group consisting of multiple systems atrophy, Parkinson's disease, and Alzheimer's disease.

1. A method of detecting a neurological disease, comprising:
contacting the brain tissue with a labeled molecule which binds multiple sites of stacked proteins associated with a neurological disease;
determining binding of the labeled molecule; and
thereby determining a neurological disease associated with the brain tissue.

2. The method of claim 1, wherein the determining comprises performing positron emission tomography, imaging mass spectrometry, or magnetic resonance imaging.

3. The method of claim 1, wherein a binding portion of the labeled molecule is characterized by:
(a) a planar core comprised of one or two rings which are optionally heterocycle;
(b) an accessible molecular conformation allowing three or more of the molecules to self-associate in a repeating, parallel-displaced stack along the stacked proteins;
(c) a configuration as shown in FIG. 3, wherein a distance between an atom within adjacent planar cores is 3.3-3.5 Å;
(d) a configuration as shown in FIG. 4, wherein a distance between equivalent atoms on two adjacent molecules is 4.8 Å;
(e) a configuration as shown in FIG. 5, wherein an angle (θ) between a line defined by two equivalent atoms on adjacent molecules and a line perpendicular to planes of the planar core is 44°;
(f) a configuration as shown in FIG. 4, wherein a minimal distance between any atom within the plane of the core and an equivalent atom within adjacent molecule bound to a stacked protein is 3.2-3.6 Å; and
(g) substituents forming non-covalent interactions with the stacked proteins which interactions are selected from the group consisting of to hydrogen bonds, Van der Waals contacts, pi-pi interactions, and chalcogen bonds.

4. The method of claim 1, wherein the neurological disease is selected from the group consisting of: Multiple Systems Atrophy, Parkinson's disease, or Alzheimer's disease.

5. The method of claim 1, wherein the labeled molecule contains one or more of [2H], [3H], [11C], [18F], or [13N].

6. The method of claim 1, wherein the stacked proteins are selected from the group consisting of α-synuclein, tau, and amyloid β.

7. The method of claim 1, wherein the labeled molecule is a compound, or a salt or a hydrate or a solvate thereof, having a structure according to formula (I): 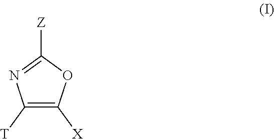
wherein
T is substituted or unsubstituted naphthyridinone or substituted or unsubstituted dihydronaphthyridinone;
X is substituted or unsubstituted aryl or substituted or unsubstituted heteroaryl, wherein X is monocyclic; and
Z is substituted or unsubstituted aryl, substituted or unsubstituted heteroaryl, substituted or unsubstituted cycloalkyl, substituted or unsubstituted heterocycloalkyl, or substituted or unsubstituted ethenyl.

8. The method of claim 7, wherein T is 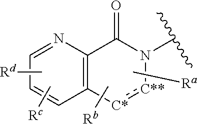
wherein R^{a}, R^{b}, R^{c}, and R^{d} are independently selected from H, halogen, substituted or unsubstituted C₁₋₃ alkyl, C₂-C₄ alkenyl, substituted or unsubstituted C₁₋₃ alkoxy, and when the connection between C* and C** is a single bond, R^{a} and R^{b} can be optionally joined with C* or with C** to form a substituted or unsubstituted cyclopropyl.

9. The method of claim 7, wherein T is

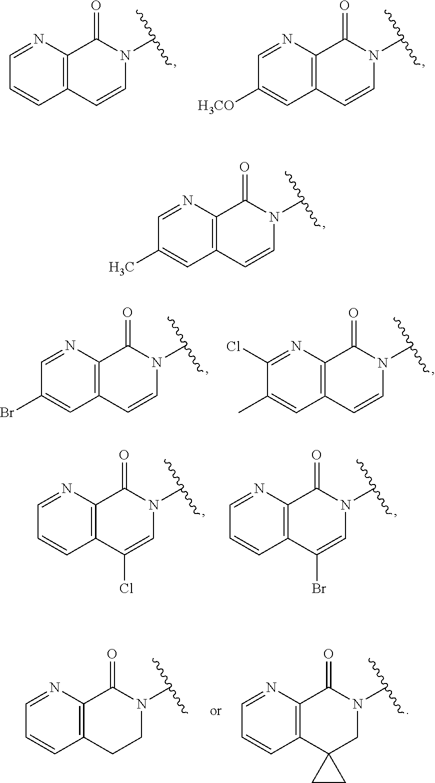

10. The method of claim 7, wherein T is

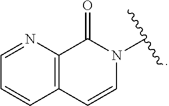 [SMILES: *n1ccc2cccnc2c1=O]

11. The method of claim 7, wherein X is substituted or unsubstituted phenyl, substituted or unsubstituted pyridinyl, substituted or unsubstituted pyrimidinyl, or substituted or unsubstituted thienyl.

12. The method of claim 7, wherein X is 4-(trifluoromethyl)phenyl, 4-fluorophenyl, 2-(trifluoromethyl)pyridin-5-yl, or 2-(1,1-difluoroethyl)pyridin-5-yl.

13. The method of claim 7, wherein Z is substituted or unsubstituted phenyl, substituted or unsubstituted pyridinyl, substituted or unsubstituted furan, substituted or unsubstituted thienyl, substituted or unsubstituted pyrimidinyl, or substituted or unsubstituted oxazolyl.

14. The method of claim 7, wherein Z is 1-methylisoquinolin-6-yl, 2-(trifluoromethyl)pyrimidin-4-yl, 2-methylpyrimidin-4-yl, 1-methyl-6-isoquinolyl, 1-(2-hydroxyethyl)-6-isoquinolyl, 3-isoquinolyl, 6-quinolyl, 8-fluoro-3-quinolyl, 8-fluoro-7-quinolyl, 4-methyl-1,7a-diaza-2-indenyl, 1-thia-5-aza-2-indenyl, 1-(2-fluoroethyl)-1H-indazol-5-yl, 3-quinolyl, 2-cyclopropyl-2H-indazol-5-yl, 6-fluoro-1,3-benzoxazol-2-yl, 5-fluoro-2-pyridyl, 1-benzofuran-2-yl, or phenyl.

15. The method of claim 7, having a structure according to formula (II), (III), (IV), (V), (VI), (VII), (VIII), or (IX):

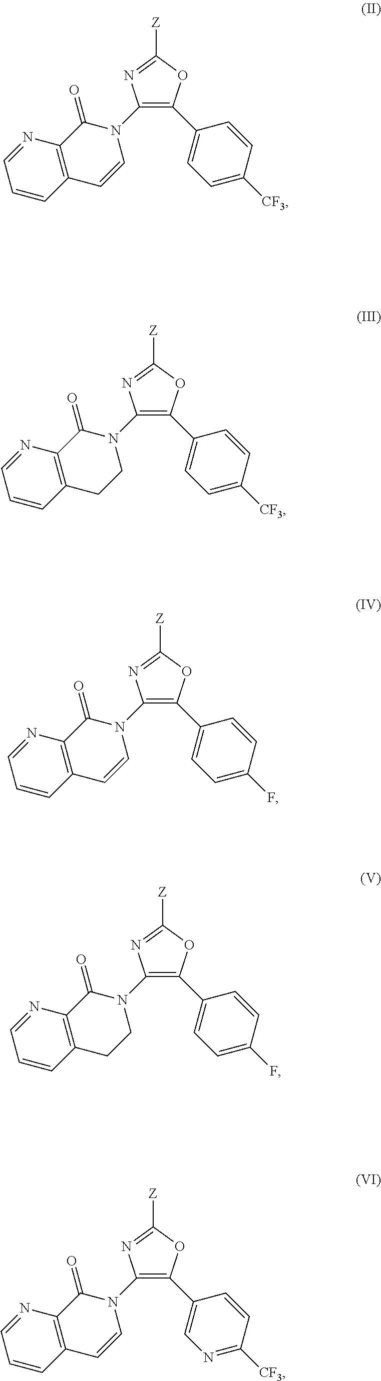

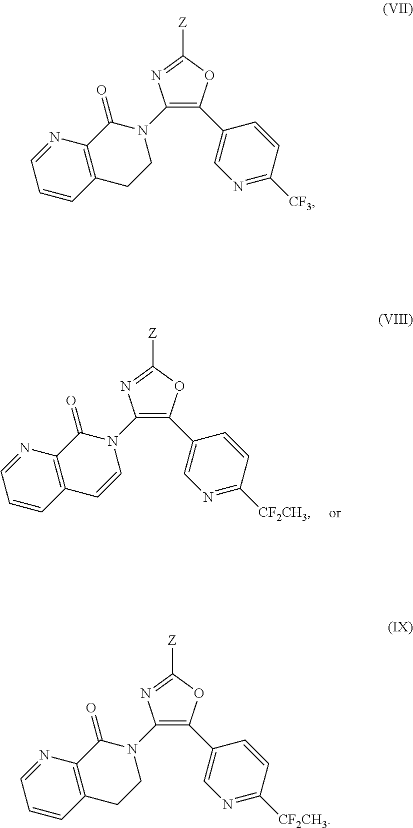

16. The method of claim 7, which is 7-{5-[6-(1,1-difluoroethyl)-3-pyridyl]-2-(1-methyl-6-isoquinolyl)-1,3-oxazol-4-yl}-1,7-diaza-8(7H)-naphthalenone, 7-{2-[1-(2-hydroxyethyl)-6-isoquinolyl]-5-[6-(trifluoromethyl)-3-pyridyl]-1,3-oxazol-4-yl}-1,7-diaza-8(7H)-naphthalenone, 7-{2-[1-(2-hydroxyethyl)-6-isoquinolyl]-5-[p-(trifluoromethyl)phenyl]-1,3-oxazol-4-yl}-1,7-diaza-8(7H)-naphthalenone, 7-[5-(p-fluorophenyl)-2-(3-isoquinolyl)-1,3-oxazol-4-yl]-1,7-diaza-8 (7H)-naphthalenone, 7-[5-(p-fluorophenyl)-2-(6-quinolyl)-1,3-oxazol-4-yl]-1,7-diaza-8 (7H)-naphthalenone, 7-{2-(8-fluoro-3-quinolyl)-5-[p-(trifluoromethyl)phenyl]-1,3-oxazol-4-yl}-1,7-diaza-8 (7H)-naphthalenone, 7-[5-(p-fluorophenyl)-2-(8-fluoro-7-quinolyl)-1,3-oxazol-4-yl]-1,7-diaza-8 (7H)-naphthalenone, 7-{2-(4-methyl-1,7a-diaza-2-indenyl)-5-[p-(trifluoromethyl)phenyl]-1,3-oxazol-4-yl}-1,7-diaza-8(7H)-naphthalenone, 7-{2-(1-thia-5-aza-2-indenyl)-5-[p-(trifluoromethyl)phenyl]-1,3-oxazol-4-yl}-1,7-diaza-8(7H)-naphthalenone, 7-{2-[1-(2-fluoro ethyl)-1H-indazol-5-yl]-5-[p-(trifluoromethyl)phenyl]-1,3-oxazol-4-yl}-1,7-diaza-8(7H)-naphthalenone, 7-{2-(3-quinolyl)-5-[p-(trifluoromethyl)phenyl]-1,3-oxazol-4-yl}-1,7-diaza-8(7H)-naphthalenone, 7-{2-(2-cyclopropyl-2H-indazol-5-yl)-5-[p-(trifluoromethyl)phenyl]-1,3-oxazol-4-yl}-1,7-diaza-8(7H)-naphthalenone, 7-{2-(6-fluoro-1,3-benzoxazol-2-yl)-5-[p-(trifluoro methyl)phenyl]-1,3-oxazol-4-yl}-1,7-diaza-8(7H)-naphthalenone, 7-{5-[p-(trifluoromethyl)phenyl]-2-[2-(trifluoromethyl)-4-pyrimidinyl]-1,3-oxazol-4-yl}-1,7-diaza-8(7H)-naphthalenone, 7-{2-(5-fluoro-2-pyridyl)-5-[p-(trifluoromethyl)phenyl]-1,3-oxazol-4-yl}-1,7-diaza-8(7H)-naphthalenone, 7-[2-(1-benzofuran-2-yl)-5-(p-fluorophenyl)-1,3-oxazol-4-yl]-1,7-diaza-8(7H)-naphthalenone, or 7-{2-phenyl-5-[p-(trifluoromethyl)phenyl]-1,3-oxazol-4-yl}-1,7-diaza-8(7H)-naphthalenone.

17. The method of claim 7, wherein the compound is 7-(2-(2-methylpyrimidin-4-yl)-5-(4-(trifluoromethyl)phenyl)oxazol-4-yl)-6,7-dihydro-1,7-naphthyridin-8(5H)-one.

18. The method of claim 7, wherein the compound is N-methyl {1-[5-(3,4-difluorophenyl)-2-(4,6-dimethyl-2-pyridyl)-1,3-oxazol-4-yl]-5-fluoro-2-oxo-1,2-dihydro-4-pyrimidinyl}amine.

19. The method of claim 1, wherein the labeled molecule is a compound of formula (XVI): 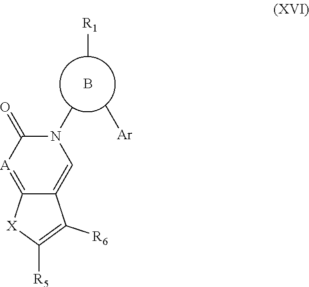
wherein:
R₁, R₅, and R₆ are each independently selected from aryl, substituted aryl, heteroaryl, substituted heteroaryl, alkyl, substituted alkyl, —CH₂R₂₀, —CHMeR₂₀, —CH(OH)R₂₀, cycloalkyl, substituted cycloalkyl, heterocycle, substituted heterocycle, alkenyl, alkynyl, acyl, nitro, halo, amino, substituted amine, ether, thioether, and H;
Ar is selected from aryl, substituted aryl, heteroaryl, and substituted heteroaryl;
A is selected from N, CH, C(halo);
X is selected from O and N—R₂,
R₂ and R₂₀ are each independently selected from aryl, substituted aryl, heteroaryl, substituted heteroaryl, alkyl, substituted alkyl, cycloalkyl, substituted cycloalkyl, heterocycle, substituted heterocycle, alkenyl, alkynyl, acyl, and H; and
Ring B is a 5-membered heteroaryl ring.

20. The method of claim 1, wherein the molecule has a structure as shown in one of the structures of FIG. 6.


SEQUENCE LISTING (9 sequences)

SEQ ID NO: 1 - protein, 352 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 2 - protein, 381 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 3 - protein, 410 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 4 - protein, 383 aa, Homo sapiens
MAEPRQEFEVMEDHAGTYGLGDRKDQGGYTMHQDQEGDTDAGLKAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 5 - protein, 412 aa, Homo sapiens
MAEPRQEFEVMEDHAGTYGLGDRKDQGGYTMHQDQEGDTDAGLKESPLQTPTEDGSEEPGSETSDAKSTPTAEAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 6 - protein, 441 aa, Homo sapiens
MAEPRQEFEVMEDHAGTYGLGDRKDQGGYTMHQDQEGDTDAGLKESPLQTPTEDGSEEPGSETSDAKSTPTAEDVTAPLVDEGAPGKQAAAQPHTEIPEGTTAEEAGIGDTPSLEDEAAGHVTQARMVSKSKDGTGSDDKKAKGADGKTKIATPRGAAPPGQKGQANATRIPAKTPPAPKTPPSSGEPPKSGDRSGYSSPGSPGTPGSRSRTPSLPTPPTREPKKVAVVRTPPKSPSSAKSRLQTAPVPMPDLKNVKSKIGSTENLKHQPGGGKVQIINKKLDLSNVQSKCGSKDNIKHVPGGGSVQIVYKPVDLSKVTSKCGSLGNIHHKPGGGQVEVKSEKLDFKDRVQSKIGSLDNITHVPGGGNKKIETHKLTFRENAKAKTDHGAEIVYKSPVVSGDTSPRHLSNVSSTGSIDMVDSPQLATLADEVSASLAKQGL

SEQ ID NO: 7 - protein, 40 aa, Homo sapiens
DAEFRHDSGYEVHHQKLVFFAEDVGSNKGAIIGLMVGGVV

SEQ ID NO: 8 - protein, 42 aa, Homo sapiens
DAEFRHDSGYEVHHQKLVFAAEDVGSNKGAIIGLMVGGVVIA

SEQ ID NO: 9 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA